(19) United States
(12) Patent Application Publication
(10) Pub. No.: US 2025/0243277 A1
(43) Pub. Date: Jul. 31, 2025

(54) PD-L1 BINDING AFFIMERS

(71) Applicant: Avacta Life Sciences Limited, Wetherby (GB)

(72) Inventors: Amrik Basran, Cambridge, Cambridgeshire (GB); Emma Jenkins, Stansted, Essex (GB); Estelle Adam, Hitchin, Hertfordshire (GB); Emma Stanley, Hitchin, Hertfordshire (GB); Michele Writer, Cambridge, Cambridgeshire (GB)

(73) Assignee: Avacta Life Sciences Limited, Wetherby (GB)

(21) Appl. No.: 18/698,618

(22) PCT Filed: Oct. 6, 2022
(86) PCT No.: PCT/EP2022/077811

§ 371 Date: Apr. 4, 2024

Related U.S. Application Data
(60) Provisional application No. 63/253,435, filed on Oct. 7, 2021.

(51) Int. Cl. C07K 16/28

(52) U.S. Cl. CPC C07K 16/2827; C07K 2317/52; C07K 2317/76; C07K 2317/92; C07K 2319/00

(57) ABSTRACT

The present disclosure relates to engineered PD-L1-binding Stefin A polypeptide variants, polynucleotides encoding the engineered PD-L1-binding Stefin A polypeptide variants, cells expressing the polypeptide variants, pharmaceutical preparations of the polypeptide variants, and uses of the polypeptide variants in the treatment of various human conditions, including cancer.

### RELATED APPLICATION

This application claims the benefit under 35 U.S.C. § 119 (e) of U.S. provisional application No. 63/253,435, filed Oct. 7, 2021, which is incorporated by reference herein in its entirety.

### REFERENCE TO AN ELECTRONIC SEQUENCE LISTING

The contents of the electronic sequence listing (A122470015WO00-SEQ-HJD.xml; Size: 1,179,900 bytes; and Date of Creation: Sep. 9, 2022) is herein incorporated by reference in its entirety.

### BACKGROUND

The PD-1 (programmed cell death-1) receptor is expressed on the surface of activated T cells. Its ligands, PD-L1 and PD-L2, are expressed on the surface of dendritic cells or macrophages. PD-1 and PD-L1/PD-L2 belong to the family of immune checkpoint proteins that act as co-inhibitory factors that can halt or limit the development of the T cell response. The PD-1/PD-L1 interaction ensures that the immune system is activated only at the appropriate time in order to minimize the possibility of chronic autoimmune inflammation. The PD-1/PD-L1 pathway represents an adaptive immune resistance mechanism used by tumor cells in response to endogenous immune anti-tumor activity. PD-L1 is overexpressed on tumor cells or on non-transformed cells in the tumor microenvironment. PD-L1 expressed on the tumor cells binds to PD-1 receptors on the activated T cells, which leads to the inhibition of the cytotoxic T cells. These deactivated T cells remain inhibited in the tumor microenvironment.

### SUMMARY

Provided herein, in some aspects, are engineered polypeptides, referred to as PD-L1 AFFIMER® polypeptides or engineered PD-L1-binding Stefin A polypeptide variants, that are based on naturally occurring proteins (Stefin A cystatin) and engineered to stably display two loops that create a binding surface with high specificity and high affinity for PD-L1. The data provided herein show that these PD-L1 AFFIMER® polypeptides bind PD-L1 with a K_{d} of less than 1×10⁻⁶M, or even less than 1×10⁻⁷M. The PD-L1 AFFIMER® polypeptides of the present disclosure are useful for targeting cells that express PD-L1. These PD-L1 AFFIMER® polypeptides have several advantages over antibodies, for example. They are smaller (˜14 kDa), simpler (no disulfide bridges and no posttranslational modifications), and more robust (thermally and chemically) than antibodies. These high affinity (single-digit nM) PD-L1 AFFIMER® polypeptides can be generated in only a few weeks, exhibit exquisite specificity, are easily modified (chemically and as fusion proteins), and are easily manufactured in bacterial, yeast, or mammalian systems with high expression yields. Further, the core AFFIMER® polypeptides are non-immunogenic.

The terms PD-L1 AFFIMER® agent and anti-PD-L1 AFFIMER® agent are used interchangeable herein.

Some aspects of the present disclosure provide a protein comprising a PD-L1 binding polypeptide that binds to PD-L1 with a Kd of 1×10⁻⁶M or less, wherein the PD-L1 binding polypeptide comprises an amino acid sequence represented by Formula (I):

FR1-(Xaa)n-FR2-(Xaa)m-FR3 (I)
wherein
   FR1 is a polypeptide sequence comprising the amino acid sequence of MIPGGLSEAK PATPEIQEIV DKVKPQLEEK TGETYGKLEA VQYKTQVX (SEQ ID NO: 1) or a polypeptide sequence having at least 70% (e.g., at least 75%, at least 80%, at least 85%, at least 90%, at least 95%, or 100%) identity to the amino acid sequence of SEQ ID NO: 1, wherein X is V or D;
FR2 is a polypeptide sequence comprising the amino acid sequence of GTNYYIKVRA GDNKYMHLKV FKSL (SEQ ID NO: 2) or a polypeptide sequence having at least 70% (e.g., at least 75%, at least 80%, at least 85%, at least 90%, at least 95%, or 100%) identity to the amino acid sequence of SEQ ID NO: 2;
FR3 is a polypeptide sequence comprising the amino acid sequence of EDLVLTGYQV DKNKDDELTG F (SEQ ID NO: 3) or a polypeptide sequence having at least 70% (e.g., at least 75%, at least 80%, at least 85%, at least 90%, at least 95%, or 100%) identity to the amino acid sequence of SEQ ID NO: 3; and
   Xaa, individually for each occurrence, is an amino acid residue; and
n and m are each, independently, an integer from 3 to 20.

In some embodiments, the PD-L1 binding polypeptide comprises an amino acid sequence having at least 90% (e.g., at least 95%, at least 96%, at least 97%, at least 98%, at least 99%, or 100%) identity to the amino acid sequence of:
MIPGGLSEAKPATPEIQEIVDKVKPQLEEKTGETYGKLEA VQYKTQVV-(Xaa)ₙ-GTNYYIKVRAGDNKYMHLKVFKSL-(Xaa)ₘ-EDLVLTGYQVDKNKDDELTGF (SEQ ID NO: 4), wherein
Xaa, individually for each occurrence, is an amino acid residue, and
n and m are each, independently, an integer from 3-20.

In some embodiments, the PD-L1 binding polypeptide comprises an amino acid sequence having at least 90% (e.g., at least 95%, at least 96%, at least 97%, at least 98%, at least 99%, or 100%) identity to the amino acid sequence of:
MIPGGLSEAKPATPEIQEIVDKVKPQLEEKTGETYGKLEAVQYKTQVD-(Xaa)ₙ-GTNYYIKVRAGDNKYMHLKVFKSL-(Xaa)ₘ-EDLVLTGYQVDKNKDDELTGF (SEQ ID NO: 5), wherein
Xaa, individually for each occurrence, is an amino acid residue, and
n and m are each, independently, an integer from 3-20.

In some embodiments, (Xaa)ₙ is an amino acid sequence selected from SEQ ID NOs: 6-259, or an amino acid sequence having at least 90% or at least 95% identity thereto.

In some embodiments, (Xaa)ₙ is an amino acid sequence selected from SEQ ID NOs: 6-259.

In some embodiments, (Xaa)ₘ is an amino acid sequence selected from SEQ ID NOs: 260-513, or an amino acid sequence having at least 90% or at least 95% identity thereto.

In some embodiments, (Xaa)ₘ is an amino acid sequence selected from SEQ ID NOs: 260-513.

In some embodiments, the PD-L1 binding polypeptide comprises an amino acid sequence having at least 90% (e.g., at least 95%, at least 96%, at least 97%, at least 98%, at least 99%, or 100%) identity to the amino acid sequence of any one of SEQ ID NOs: 514-767.

In some embodiments, the PD-L1 binding polypeptide comprises the amino acid sequence of any one of SEQ ID NOs: 514-767.

In some embodiments, the PD-L1 binding polypeptide is encoded by a polynucleotide comprising a nucleotide sequence having at least 90% (e.g., at least 95%, at least 96%, at least 97%, at least 98%, at least 99%, or 100%) identity to the nucleotide sequence of any one of SEQ ID NOs: 768 to 1021, 1126, 1128, 1130, 1132, 1134, 11336, 1138, 1140, 1142, 1144, 1146, 1148, 1150, 1152, 1154, 1156, 1158, 1160, 1162, 1163, 1165, 1166, and 1168.

Other aspects of the present disclosure provide a fusion protein comprising a homodimer of the protein of any one of the preceding paragraphs.

Yet other aspects of the present disclosure provide a fusion protein comprising the protein of any one of the preceding paragraphs and a soluble receptor, a growth factor, a cytokine, a chemokine, a costimulatory agonist, or a checkpoint inhibitor.

Still other aspects of the present disclosure provide a fusion protein comprising the protein of any one of the preceding paragraphs and a half-life extending polypeptide.

In some embodiments, the half-life extending polypeptide is selected from the group consisting of an Fc domain, an albumin protein, an albumin-binding polypeptide, transferrin, a transferrin-binding polypeptide, fibronectin, or a fibronectin-binding polypeptide.

In some embodiments, the half-life extending polypeptide is an Fc domain.

In some embodiments, the fusion protein further comprises a linker, optionally a flexible linker or a rigid linker.

In some embodiments, the protein (i) comprises an amino acid sequence having at least 90% identity to the amino acid sequence of SEQ ID NO: 1122 or (ii) is encoded by a polynucleotide comprising a nucleotide sequence having at least 90% identity to the nucleotide sequence of SEQ ID NO: 1166.

In some embodiments, the protein (i) comprises the amino acid sequence of SEQ ID NO: 1122 or (ii) is encoded by a polynucleotide comprising the nucleotide sequence of SEQ ID NO: 1166.

In some embodiments, the protein (i) comprises an amino acid sequence having at least 90% identity to the amino acid sequence of SEQ ID NO: 1121 or (ii) is encoded by a polynucleotide comprising a nucleotide sequence having at least 90% identity to the nucleotide sequence of SEQ ID NO: 1163.

In some embodiments, the protein (i) comprises the amino acid sequence of SEQ ID NO: 1121 or (ii) is encoded by a polynucleotide comprising the nucleotide sequence of SEQ ID NO: 1163.

Some aspects of the present disclosure provide a recombinant antibody comprising a VH and/or VL chains forming an antigen binding sites that bind to a target antigen, wherein at least one of the VH and/or VL chains is a fusion protein comprising the protein of any one of the preceding paragraphs.

In some embodiments, the target antigen is selected from the group consisting of an immune checkpoint, an immune costimulatory receptor, an angiogenic factor, and a tumor antigen.

In some embodiments, the target antigen is selected from the group consisting of PD-1, PD-L2, CTLA-4, NKG2A, KIR, LAG-3, TIM-3, CD96, VISTA, TIGIT, CD28, ICOS, CD137, OX40, GITR, CD27, CD30, HVEM, DNAM-1 or CD28H, CEACAM-1, CEACAM-5, BTLA, LAIR1, CD160, 2B4, TGFR, B7-H3, B7-H4, CD40, CD40L, CD47, CD70, CD80, CD86, CD94, CD137, CD137L, CD226, Galectin-9, GITRL, HHLA2, ICOS, ICOSL, LIGHT, MHC class I or II, NKG2a, NKG2d, OX4OL, PVR, SIRPα, TCR, CD20, CD30, CD33, CD38, CD52, VEGF, VEGF receptors, EGFR, Her2/neu, ILT1, ILT2, ILT3, ILT4, ILT5, ILT6, ILT7, ILT8, KIR2DL1, KIR2DL2, KIR2DL3, KIR2DL4, KIR2DLSA, KIR2DL5B, KIR3DL1, KIR3DL2, KIR3DL3, NKG2A, NKG2C, NKG2E or TSLP.

Other aspects of the present disclosure provide a recombinant receptor trap fusion protein comprising (i) a ligand binding domain of a receptor, and (ii) the protein of any one of the preceding paragraphs.

In some embodiments, the ligand binding domain binds to PGE2, TGF-β, VEGF, CCL2, IDO, CSF1, IL-10, IL-13, IL-23, or adenosine.

Yet other aspects of the present disclosure provide a recombinant receptor ligand fusion protein comprising (i) a polypeptide ligand sequence that binds to an agonizes or antagonizes its cognate receptor, and (ii) the protein of any one of the preceding paragraphs.

In some embodiments, the polypeptide ligand is a ligand for a co-stimulatory receptor and agonizes the co-stimulatory receptor upon binding.

In some embodiments, the polypeptide ligand is selected from B7.1, 4-1BBL, OX40L, GITRL or LIGHT.

In some embodiments, the protein further includes a multimerization domain that induces multimerization of the recombinant receptor ligand fusion protein.

In some embodiments, the polypeptide ligand is an immunostimulatory cytokine that promotes antitumor immunity.

In some embodiments, the polypeptide ligand is selected from IFN-α2, IL-2, IL-15, IL-21, and IL-12.

Further aspects of the present disclosure provide a multispecific T-cell engaging fusion protein comprising (i) a CD3 binding polypeptide binds to CD3 on the surface of T-cells, and (ii) the protein of any one of the preceding paragraphs.

Some aspects of the present disclosure provide a chimeric receptor fusion protein comprising (i) an extracellular portion including protein of any one of the preceding paragraphs; (ii) a transmembrane domain; and (iii) a cytoplasmic domain comprising a 4-1BB signaling domain and a CD3& signaling domain, and optionally a costimulatory signaling region.

Other aspects of the present disclosure provide a polynucleotide comprising a nucleotide sequence encoding the protein of any one of the preceding paragraphs.

In some embodiments, the polynucleotide comprises a nucleotide sequence having at least 90% (e.g., at least 95%, at least 96%, at least 97%, at least 98%, at least 99%, or 100%) identity to the nucleotide sequence of any one of SEQ ID NOs: 768 to 1021.

In some embodiments, the polynucleotide comprises the nucleotide sequence of any one of SEQ ID NOs: 768 to 1021.

Some aspects of the present disclosure provide a vector, optionally a viral vector or a plasmid vector, comprising the polynucleotide of any one of the preceding paragraphs.

Some aspects of the present disclosure provide a cell, optionally a mammalian cell, comprising the polynucleotide of any one of the preceding paragraphs or the vector of the preceding paragraph.

Other aspects of the present disclosure provide a pharmaceutical composition comprising: (a) the protein of any one of the preceding paragraphs, the fusion protein of any one of the preceding paragraphs, the recombinant antibody of any one of the preceding paragraphs, the recombinant receptor trap fusion protein of any one of the preceding paragraphs, the recombinant receptor ligand fusion protein of any one of the preceding paragraphs, the multispecific T-cell engaging fusion protein of any one of the preceding paragraphs, the chimeric receptor fusion protein of any one of the preceding paragraphs, the polynucleotide of any one of the preceding paragraphs, the vector of any one of the preceding paragraphs, or the cell of c of any one of the preceding paragraphs; and (b) a pharmaceutically acceptable excipient.

Still other aspects of the present disclosure provide a method comprising administering to a subject the pharmaceutical composition of the preceding paragraph.

In some embodiments, the subject has a cancer.

In some embodiments, the pharmaceutical composition is administered subcutaneously, intravenously, or intramuscularly.

### BRIEF DESCRIPTION OF THE DRAWINGS

FIGS. 1A-1B shows an anti-PD-L1 monomer type III AFFIMER® characterization, including a gel and table showing the percent monomer formation (FIG. 1A) and graphs demonstrating the percent monomer formation of selected clones (FIG. 1B).

FIG. 2 is a human PD-L1-Fc BIACORE™ kinetic analysis, including examples of three type III AFFIMER® monomers.

FIG. 3 is two PD-L1/PD1 competition ELISAs screening four type III AFFIMER® monomers.

FIG. 4 shows the results of a Promega PD1/PD-L1 blockade cell-based assay of two type III AFFIMER® monomers compared to two type I AFFIMER® monomers.

FIG. 5 shows the results of a Promega PD1/PD-L1 blockade cell-based assay comparing type III AFFIMER® monomers to clinical monoclonal antibodies (mAbs).

FIGS. 6A-6C show an assessment of anti-PD-L1 AFFIMER® cell binding to a PD-L1 overexpressing Chinese hamster ovary (CHO) cell line. Binding occurs in the PD-L1 overexpressing CHO cell line (FIG. 6A) but does not occur in the CHO cell line (not expressing PD-L1) (FIG. 6B). Representative histograms at 100 nM are shown for the three clones tested (FIG. 6C).

FIG. 7A-7G are Clone 80 mutant comparisons performed using Promega PD1/PD-L1 blockade cell-based assay.

FIGS. 8A-8B show a quality control (QC) analysis of the of PD-L1 domain/Clone 80 AFFIMER® antigen complex to be used for crystallography studies, including SEC-HPLC (FIG. 8A) and SDS-PAGE (FIG. 8B).

FIGS. 9A-9B are schematics depicting the solved crystal structure of the PD-L1 domain/Clone 80 AFFIMER® antigen complex and interaction surface.

FIGS. 10A-10C show the formatting of AFFIMER® Clone 127 as a human IgG1 Fc fusion format. FIG. 10A shows a SEC analysis; FIG. 10B depicts a BIACORE™ kinetic analysis; and FIG. 10C shows the results of a competition ELISA.

FIG. 11 shows the mammalian HEK expression of the AFFIMER® Clone 80 monomer (top) and in-line fusion (ILF) homodimer format (bottom). T=monomer; CH=in-line fusion

FIG. 12 is two graphs showing a human PD-L1-Fc BIACORE™ kinetic analysis of mammalian expressed AFFIMER® Clone 80 monomer (top) and ILF fused homodimer format (bottom).

FIG. 13 shows the results of a PD-L1 binding ELISA using mammalian-produced AFFIMER® Clone 80 in the ILF homodimer format.

FIG. 14 shows the results of a Promega PD1/PD-L1 blockade cell-based assay in which the mammalian-expressed AFFIMER® Clone 80 monomer and ILF homodimer format were compared.

FIG. 15 shows the results of a staphylococcal enterotoxin B (SEB; Toxin Technology) primary cell-based assay using the AFFIMER® Clone 80 homodimer ILF and peripheral blood monocyte cells (PBMCs) from two healthy human donors.

FIG. 16 shows the results of a cysteine protease inhibition assay using the AFFIMER® Clone 80 monomer.

FIG. 17 shows the results of a melting temperature analysis using a nano differential scanning calorimeter (nDSC) for the AFFIMER® Clone 80.

### DETAILED DESCRIPTION

### I. Overview

Cancer immunotherapy has been accompanied by promising results over the past few years. Programmed Cell Death Protein 1 (PD-1) plays a vital role in inhibiting immune responses and promoting self-tolerance through modulating the activity of T-cells, activating apoptosis of antigen-specific T cells and inhibiting apoptosis of regulatory T cells. Programmed Cell Death Ligand 1 (PD-L1) is a trans-membrane protein that is considered to be a co-inhibitory factor of the immune response, it can combine with PD-1 to reduce the proliferation of PD-1 positive cells, inhibit their cytokine secretion and induce apoptosis. PD-L1 also plays an important role in various malignancies where it can attenuate the host immune response to tumor cells. Based on these perspectives, PD-1/PD-L1 axis is responsible for cancer immune escape and makes a huge effect on cancer therapy.

PD-1/PD-L1 pathway controls the induction and maintenance of immune tolerance within the tumor microenvironment. The activity of PD-1 and its ligands PD-L1 or PD-L2 are responsible for T cell activation, proliferation, and cytotoxic secretion in cancer to degenerating anti-tumor immune responses.

PD-1 ligand (PD-L1; also referred to as CD279 and B7-H1), belongs to the B7 series and is a 33-kDa type 1 transmembrane glycoprotein that contains 290 amino acids with Ig- and IgC domains in its extracellular region.

PD-L1 is usually expressed by macrophages, some activated T cells and B cells, dendritic cells (DCs) and some epithelial cells, particularly under inflammatory conditions [18]. In addition, PD-L1 is expressed by tumor cells as an “adaptive immune mechanism” to escape anti-tumor responses. PD-L1 is associated with an immune environment rich in CD8 T cells, production of Th1 cytokines and chemical factors, as well as interferons and specific gene expression characteristics. It has been demonstrated that interferon-gamma (IFN-γ) causes PD-L1 upregulation in ovarian cancer cells, which is responsible for disease progression, whereas IFN-γ receptor 1 inhibition can reduce PD-L1 expression in acute myeloid leukemia mouse models through the MEK/extracellular signal-regulated kinase (ERK) and MYD88/TRAF6 pathways. IFN-γ induces protein kinase D isoform 2 (PKD2), which is important for the regulation of PD-L1. Inhibition of PKD2 activity inhibits the expression of PD-L1 and promotes a strong antitumor immune response. NK cells secrete IFN-γ through the Janus kinase (JAK) 1, JAK2 and signal transducer and activator of transcription (STAT) 1 pathways, increasing the expression of PD-L1 on the surface of the tumor cells. Studies on melanoma cells have shown that IFN-γ secreted by T cells through the JAK1/JAK2-STAT1/STAT2/STAT3-IRF1 pathway may regulate the expression of PD-L1. T and NK cells appear to secrete IFN-γ, which induces PD-L1 expression on the surface of the target cells, including tumor cells.

PD-L1 acts as a pro-tumorigenic factor in cancer cells via binding to its receptors and activating proliferative and survival signaling pathways. This finding further indicated that PD-L1 is implicated in subsequent tumor progression. In addition, PD-L1 has been shown to exert non-immune proliferative effects on a variety of tumor cell types. For example, PD-L1 induced epithelial-to-mesenchymal transition (EMT) and stem cell-like phenotypes in renal cancer cells, indicating that the presence of the intrinsic pathway of PD-L1 promotes kidney cancer progression.

The present disclosure addresses the urgent need in the art for targeting molecules capable of binding to PD-L1 with high specificity and high affinity. Provided herein are PD-L1 AFFIMER® polypeptides, engineered polypeptide variants of the Stefin A protein, that bind PD-L1 with a K_{d} of less than 1×10⁻⁶M. The PD-L1 AFFIMER® polypeptides of the present disclosure, in some embodiments, may be fused or otherwise linked to therapeutic molecules to be used for the treatment of diseases and/or disorders characterized at least in part by the presence of PD-L1-positive cells. In other embodiments, the PD-L1 AFFIMER® polypeptides can be used as therapeutic agents.

### II. Certain Definitions of the Present Disclosure

Stefin polypeptides encompass a subgroup of proteins in the cystatin superfamily, a family which encompasses proteins that contain multiple cystatin-like sequences. The Stefin subgroup of the cystatin family includes relatively small (around 100 amino acids) single domain proteins. They receive no known post-translational modification, and lack disulfide bonds, suggesting that they will be able to fold identically in a wide range of extracellular and intracellular environments. Stefin A itself is a monomeric, single chain, single domain protein of 98 amino acids. The structure of Stefin A has been solved, facilitating the rational mutation of Stefin A into the AFFIMER® polypeptide. The only known biological activity of cystatins is the inhibition of cathepsin activity, which allowed for exhaustive testing for residual biological activity of the engineered proteins.

An “AFFIMER® polypeptide” (also referred to as an “AFFIMER® protein”) refers to a small, highly stable protein that is an engineered variant of a Stefin polypeptide. AFFIMER® proteins display two peptide loops and an N-terminal sequence that can all be randomized to bind to desired target proteins with high affinity and specificity, in a similar manner to monoclonal antibodies. Stabilization of the two peptides by the Stefin A protein scaffold constrains the possible conformations that the peptides can take, increasing the binding affinity and specificity compared to libraries of free peptides. These engineered non-antibody binding proteins are designed to mimic the molecular recognition characteristics of monoclonal antibodies in different applications. Variations to other parts of the Stefin A polypeptide sequence can be carried out, with such variations improving the properties of these affinity reagents, such as increase stability, make them robust across a range of temperatures and pH and the like. In some embodiments, an AFFIMER® polypeptide includes a sequence derived from Stefin A, sharing substantial identify with a Stefin A wild type sequence, such as human Stefin A. It will be apparent to a person skilled in the art that modifications may be made to the scaffold sequence without departing from the disclosure. In particular, an AFFIMER® polypeptide can have an amino acid sequences that is at least 25%, 35%, 45%, 55% or 60% identity to the corresponding sequences to human Stefin A, for example, at least 70%, at least 80%, at least 85%, at least 90%, at least 92%, at least 94%, at least 95% identical, e.g., where the sequence variations do not adversely affect the ability of the scaffold to bind to the desired target (such as PD-L1), and e.g., which do not restore or generate biological functions such as those which are possessed by wild type Stefin A but which are abolished in mutational changes described herein.

An “AFFIMER® agent” refers to a polypeptide that includes an AFFIMER® polypeptide sequence and any other modification(s) (e.g., conjugation, post-translational modifications, etc.) so as to represent a therapeutically active protein intended for delivery to an individual.

An “AFFIMER®-linked conjugate” refers to an AFFIMER® agent having at least one moiety conjugated thereto through a chemical conjugation other than through the formation of a contiguous peptide bond through the C-terminus or N-terminus of the polypeptide portion of the AFFIMER® agent containing AFFIMER® polypeptide sequence. An AFFIMER®-linked conjugate may be an “AFFIMER® polypeptide-drug conjugate”, which refers to an AFFIMER® agent including at least one pharmacologically active moiety conjugated thereto. An AFFIMER®-linked conjugate may also be an “AFFIMER®-tag conjugate”, which refers to an AFFIMER® agent including at least one detectable moiety (e.g., detectable label) conjugated thereto.

An “encoded AFFIMER® construct” refers to a nucleic acid construct which, when expressed by cells in a patient's body through a gene delivery process, produces an intended AFFIMER® agent in vivo.

Programmed death-ligand 1 (PD-L1), also known as cluster of differentiation 274 (CD274) or B7 homolog 1 (B7-H1), is a protein that in humans is encoded by the (I) 274 gene. PD-L1 is a 40 kDa type 1 transmembrane protein that is expressed by various tumor cells and by the lymphocytes that infiltrate tumors. PD-L1 is expressed on the surface of tumor cells and it is able to bind to PD-1 on the surface of activated T cells, B cells, and myeloid cells, to modulate activation or inhibition. The binding of PD-L1 to PD-1 leads to an immunosuppressive effect and allows the tumor to evade immune destruction. The affinity between PD-L1 and PD-1, as defined by the dissociation constant K_{d}, is 770 nM. PD-L1 also has an appreciable affinity for the costimulatory molecule CD80 (B7-1), but not CD86 (B7-2).

PD-L1 has been speculated to play a major role in suppressing the adaptive arm of immune system during particular events such as pregnancy, tissue allografts, autoimmune disease and other disease states such as hepatitis. Normally the adaptive immune system reacts to antigens that are associated with immune system activation by exogenous or endogenous danger signals. In turn, clonal expansion of antigen-specific CD8+ T cells and/or CD4+ helper cells is propagated. The binding of PD-L1 to the inhibitory checkpoint molecule PD-1 transmits an inhibitory signal based on interaction with phosphatases (SHP-1 or SHP-2) via Immunoreceptor Tyrosine-Based Switch Motif (ITSM). This reduces the proliferation of antigen-specific T-cells in lymph nodes, while simultaneously reducing apoptosis in regulatory T cells (anti-inflammatory, suppressive T cells)—further mediated by a lower regulation of the gene Bcl-2.

The human amino acid and nucleic acid sequences can be found in a public database, such as GenBank, UniProt and Swiss-Prot. For example, the amino acid sequence of human PD-L1 can be found as UniProt/Swiss-Prot. Accession No. Q9NZQ7-1 and the nucleotide sequence encoding of the human PD-L1 can be found at NCBI Accession No. NM_014143.4 (Gene ID: 29126). As used herein, “PD-L1” includes any native, mature PD-L1 which results from processing of a PD-L1 precursor protein in a cell. The term encompasses PD-L1 from any vertebrate source, including mammals such as primates (e.g., humans and cynomolgus monkeys) and rodents (e.g., mice and rats), unless otherwise indicated. The term also includes any PD-L1 proteins comprising mutations, e.g., point mutations, fragments, insertions, deletions, and splice variants of full length wild-type PD-L1.

A “PD-L1 AFFIMER® agent” refers to an AFFIMER® agent that comprises at least one AFFIMER® polypeptide that binds to PD-L1, particularly human PD-L1, with a dissociation constant (Kd) of at least 10⁻⁶M. In some embodiments, the PD-L1 AFFIMER® agent binds PD-L1 with a Kd of 1×10⁻⁷M or less, Kd of 1×10⁻⁸M or less, Kd of 1×10⁻⁹ M or less, or a Kd of 1×10⁻¹⁰ M or less. It should be understood that the terms “PD-L1 AFFIMER® polypeptide” and “engineered PD-L1-binding Stefin A polypeptide variant” are used interchangeably herein. Thus, a “PD-L1 AFFIMER® polypeptide” is an engineered polypeptide that binds specifically to PD-L1 with a K_{d} of 1×10⁻⁶M or less, wherein the engineered polypeptide is a variant of a Stefin A protein.

### A. Polypeptides

Polypeptides (which includes peptides and proteins) are polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing at least one analog of an amino acid (including, for example, unnatural amino acids), as well as other modifications known in the art.

Amino acids (also referred to herein as amino acid residues) participate in one more peptide bonds of a polypeptide. In general, the abbreviations used herein for designating the amino acids are based on recommendations of the IUPAC-IUB Commission on Biochemical Nomenclature (see Biochemistry (1972) 11:1726-1732). For instance, Met, Ile, Leu, Ala and Gly represent “residues” of methionine, isoleucine, leucine, alanine and glycine, respectively. By the residue is meant a radical derived from the corresponding α-amino acid by eliminating the OH portion of the carboxyl group and the H portion of the α-amino group. The term “amino acid side chain” is that part of an amino acid exclusive of the —CH(NH2)COOH portion, as defined by K. D. Kopple, “Peptides and Amino Acids”, W. A. Benjamin Inc., New York and Amsterdam, 1966, pages 2 and 33.

For the most part, the amino acids used in the application of this disclosure are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan, and those amino acids and amino acid analogs which have been identified as constituents of peptidylglycan bacterial cell walls.

Amino acid residues having “basic sidechains” include Arg, Lys and His. Amino acid residues having “acidic sidechains” include Glu and Asp. Amino acid residues having “neutral polar sidechains” include Ser, Thr, Asn, Gln, Cys and Tyr. Amino acid residues having “neutral non-polar sidechains” include Gly, Ala, Val, Ile, Leu, Met, Pro, Trp and Phe. Amino acid residues having “non-polar aliphatic sidechains” include Gly, Ala, Val, Ile and Leu. Amino acid residues having “hydrophobic sidechains” include Ala, Val, Ile, Leu, Met, Phe, Tyr and Trp. Amino acid residues having “small hydrophobic sidechains” include Ala and Val. Amino acid residues having “aromatic sidechains” include Tyr, Trp and Phe.

Amino acid residues further include analogs, derivatives and congeners of any specific amino acid referred to herein, as for instance, the subject AFFIMER® polypeptides (particularly if generated by chemical synthesis) can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminiopimelic acid, ornithine, or diaminobutyric acid. Other naturally occurring amino acid metabolites or precursors having side chains which are suitable herein will be recognized by those skilled in the art and are included in the scope of the present disclosure.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this disclosure includes asymmetric carbon atoms. It is to be understood accordingly that the isomers arising from such asymmetry are included within the scope of this disclosure. Such isomers can be obtained in substantially pure form by classical separation techniques and by sterically controlled synthesis. For the purposes of this application, unless expressly noted to the contrary, a named amino acid shall be construed to include both the (D) or (L) stereoisomers.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A conservative amino acid substitution is one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

A polypeptide, soluble protein, antibody, polynucleotide, vector, cell, or composition which is “isolated” is a polypeptide, soluble protein, antibody, polynucleotide, vector, cell, or composition which is in a form not found in nature. Isolated polypeptides, soluble proteins, antibodies, polynucleotides, vectors, cells, or compositions include those which have been purified to a degree that they are no longer in a form in which they are found in nature. In some embodiments, a polypeptide, soluble protein, antibody, polynucleotide, vector, cell, or composition which is isolated is substantially pure.

A material is considered substantially pure if the material is at least 50% pure (e.g., free from contaminants), at least 90% pure, at least 95% pure, at least 98% pure, or at least 99% pure.

A fusion polypeptide (e.g., a fusion protein) is a hybrid polypeptide expressed by a nucleic acid molecule comprising at least two open reading frames (e.g., from two individual molecules, e.g., two individual genes).

A linker (also referred to as a linker region) may be inserted between a first polypeptide (e.g., copies of a PD-L1 AFFIMER® polypeptide) and a second polypeptide (e.g., another AFFIMER® polypeptide, an Fc domain, a ligand binding domain, etc.). In some embodiments, a linker is a peptide linker. Linkers should not adversely affect the expression, secretion, or bioactivity of the polypeptides. In some embodiments, linkers are not antigenic and do not elicit an immune response.

An “AFFIMER® polypeptide-antibody fusion” is a fusion protein that includes an AFFIMER® polypeptide portion and a variable region of an antibody. AFFIMER® polypeptide-antibody fusions may include full length antibodies having, for example, at least one AFFIMER® polypeptide sequence appended to the C-terminus or N-terminus of at least one of its VH and/or VL chains, e.g., at least one chain of the assembled antibody is a fusion protein with an AFFIMER® polypeptide. AFFIMER® polypeptide-antibody fusions may also include at least one AFFIMER® polypeptide sequence as part of a fusion protein with an antigen binding site or variable region of an antibody fragment.

An antibody is an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term “antibody” encompasses intact (whole) polyclonal antibodies, intact monoclonal antibodies, antibody fragments (such as Fab, Fab′, F(ab′)2, and Fv fragments), single chain Fv (scFv) antibodies provided those fragments have been formatted to include an Fc or other FcγRIII binding domain, multispecific antibodies, bispecific antibodies, monospecific antibodies, monovalent antibodies, chimeric antibodies, humanized antibodies, human antibodies, fusion proteins comprising an antigen-binding site of an antibody (formatted to include an Fc or other FcγRIII binding domain), antibody mimetics, and any other modified immunoglobulin molecule comprising an antigen-binding site as long as the antibodies exhibit the desired biological activity.

While the antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu.

A variable region of an antibody may be a variable region of an antibody light chain or a variable region of an antibody heavy chain, either alone or in combination. Generally, the variable region of heavy and light chains includes four framework regions (FR) and three complementarity determining regions (CDRs), also known as hypervariable regions. The CDRs in each chain are held together in close proximity by the framework regions and, with the CDRs from the other chain, contribute to the formation of the antigen-binding sites of the antibody. There are at least two techniques for determining CDRs: (1) an approach based on cross-species sequence variability (e.g., Kabat et al., 1991, Sequences of Proteins of Immunological Interest, 5th Edition, National Institutes of Health, Bethesda Md.), and (2) an approach based on crystallographic studies of antigen-antibody complexes (A1 Lazikani et al., 1997, J. Mol. Biol., 273:927-948). In addition, combinations of these two approaches are sometimes used in the art to determine CDRs.

A humanized antibody is a form of a non-human (e.g., murine) antibody that is specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability. In some instances, the Fv framework region residues of a human immunoglobulin are replaced with the corresponding residues in an antibody from a non-human species. The humanized antibody can be further modified by the substitution of additional residues either in the Fv framework region and/or within the replaced non-human residues to refine and optimize antibody specificity, affinity, and/or binding capability. The humanized antibody may comprise variable domains containing all or substantially all of the CDRs that correspond to the non-human immunoglobulin whereas all or substantially all of the framework regions are those of a human immunoglobulin sequence. In some embodiments, the variable domains comprise the framework regions of a human immunoglobulin sequence. In some embodiments, the variable domains comprise the framework regions of a human immunoglobulin consensus sequence. The humanized antibody can also comprise at least a portion of an immunoglobulin constant region or domain (Fc), typically that of a human immunoglobulin. A humanized antibody is usually considered distinct from a chimeric antibody.

An epitope (also referred to herein as an antigenic determinant) is the portion of an antigen capable of being recognized and specifically bound by a particular antibody, a particular AFFIMER® polypeptide or other particular binding domain. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

“Specifically binds to” or is “specific for” refers to measurable and reproducible interactions such as binding between a target (e.g., PD-L1) and an AFFIMER® polypeptide, antibody or other binding partner, which is determinative of the presence of the target in the presence of a heterogeneous population of molecules including biological molecules. For example, an AFFIMER® polypeptide that specifically binds to PD-L1 is an AFFIMER® polypeptide that binds PD-L1 with greater affinity, avidity (if multimeric formatted), more readily, and/or with greater duration than it binds to other targets.

“Conjugate,” “conjugation” and grammatical variations thereof refers the joining or linking together of two or more compounds resulting in the formation of another compound, by any joining or linking methods known in the art. It can also refer to a compound that is generated by the joining or linking together two or more compounds. For example, a PD-L1 AFFIMER® polypeptide linked directly or indirectly to at least one chemical moiety or polypeptide is an exemplary conjugate. Such conjugates include fusion proteins, those produced by chemical conjugates and those produced by any other methods.

### B. Polynucleotides

A polynucleotide (also referred to herein as a nucleic acid or a nucleic acid molecule) is a polymer of nucleotides of any length and may comprise DNA, RNA (e.g., messenger RNA (mRNA)) or a combination of DNA and RNA. The nucleotides can be deoxyribonucleotides, ribonucleotides, modified nucleotides or bases, and/or their analogs, or any substrate that can be incorporated into a polymer by DNA or RNA polymerase.

A polynucleotide encoding a polypeptide refers to the order or sequence of nucleotides along a strand of deoxyribonucleic acid deoxyribonucleotides. The order of these deoxyribonucleotides determines the order of amino acids along the polypeptide (e.g., protein) chain. Thus, a nucleic acid sequence encodes the amino acid sequence.

When used in reference to nucleotide sequences, a “sequence” may comprise DNA and/or RNA (e.g., messenger RNA) and may be single and/or double stranded.

Nucleic acid sequences may be modified, e.g., mutated, relative to naturally occurring nucleic acid sequences, for example.

Nucleic acid sequence may have any length, for example 2 to 000,000 or more nucleotides (or any integral value above or between) a nucleic acid, for example a length of from about 100 to about 10,000, or from about 200 nucleotides to about 500 nucleotides.

Transfection is the process of introducing an exogenous nucleic acid into a eukaryotic cell. Transfection can be achieved by various means known in the art, including calcium phosphate-DNA co-precipitation, DEAE-dextran-mediated transfection, polybrene-mediated transfection, electroporation, microinjection, liposome fusion, lipofection, protoplast fusion, retroviral infection, and biolistics technology (biolistics).

A vector is a construct that is capable of delivering, and usually expressing, at least one gene or sequence of interest in a host cell. Examples of vectors include but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes. A vector may, in some embodiments, be an isolated nucleic acid that can be used to deliver a composition to the interior of the cell. It is known in the art a number of vectors including, but not limited to the linear polynucleotides, polynucleotides associated with ionic or amphiphilic compounds, plasmids, and viruses. Thus, a vector may be an autonomously replicating plasmid or virus. The term should also be construed to include facilitate transfer of nucleic acid into cells of the non-plasmid and non-viral compounds, for example, polylysine compounds, liposomes, and the like. Non-limiting examples of viral vectors include but are not limited to adenoviral vectors, adeno-associated virus vectors, and retroviral vectors.

An expression vector is a vector comprising a recombinant polynucleotide comprising expression control sequence and a nucleotide sequence to be expressed operably linked. The expression vector comprises sufficient cis-acting elements (cis-acting elements) used for expression; other elements for expression can be supplied by the host cell or in vitro expression system. Expression vectors include, for example, cosmids, plasmids (e.g., naked or contained in liposomes) and viruses (e.g., lentivirus, retroviruses, adenoviruses and adeno-associated viruses).

Operably linked refers to functional linkage between the regulatory sequence and a heterologous nucleic acid sequence resulting in the expression of the latter. For example, if the promoter affects the transcription or expression of the coding sequence, the promoter is operably linked to a coding sequence. Typically, DNA sequencing operably linked are contiguous, and may join two protein coding regions in the same reading frame.

A promoter is a DNA sequence recognized by the synthetic machinery required for the synthesis machinery of the cell specific transcription of a polynucleotide sequence or introduced.

Inducible expression refers to expression under certain conditions, such as activation (or inactivation) of an intracellular signaling pathway or the contacting of the cells harboring the expression construct with a small molecule that regulates the expression (or degree of expression) of a gene operably linked to an inducible promoter sensitive to the concentration of the small molecule. This is contrasted with constitutive expression, which refers to expression under physiological conditions (not limited by certain conditions).

Electroporation refers to the use of a transmembrane electric field pulse to induce microscopic pathways (pores) in a bio-membrane; their presence allows biomolecules such as plasmids or other oligonucleotide to pass from one side of the cellular membrane to the other.

### C. Checkpoint Inhibitors, Co-Stimulatory Agonists and Chemotherapeutics

A checkpoint molecule is a protein that is expressed by tissues and/or immune cells and reduce the efficacy of an immune response in a manner dependent on the level of expression of the checkpoint molecule. When these proteins are blocked, the “brakes” on the immune system are released and, for example, T cells are able to kill cancer cells more effectively. Examples of checkpoint proteins found on T cells or cancer cells include PD-1/PD-L1 and CTLA-4/B7-1/B7-2, PD-L2, NKG2A, KIR, LAG-3, TIM-3, CD96, VISTA and TIGIT.

A checkpoint inhibitor is a drug entity that reverses the immunosuppressive signaling from a checkpoint molecule.

A costimulatory molecule is an immune cell such as a T cell cognate binding partner that specifically binds to costimulatory ligands thereby mediating co-stimulation, such as, but not limited to proliferation. Costimulatory molecules are cell surface molecules other than the antigen receptor or ligand which facilitate an effective immune response. Co-stimulatory molecules include but are not limited to MHCI molecules, BTLA receptor and Toll ligands, and OX40, CD27, CD28, CDS, ICAM-1, LFA-1 (CD11a/CD18), ICOS (CD278) and 4-1BB (CD137). Examples of costimulatory molecules include but are not limited to: CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and CD83 ligand.

A costimulatory agonist is a drug entity that activates (agonizes) the costimulatory molecule, such as costimulatory ligand would do, and produces an immunostimulatory signal or otherwise increases the potency or efficacy of an immune response.

A chemotherapeutic agent is a chemical compound useful in the treatment of cancer. Examples of chemotherapeutic agents include alkylating agents such as thiotepa and cyclophosphamide (CYTOXAN); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL); beta-lapachone; lapachol; colchicines; betulinic acid; a camptothecin (including the synthetic analogue topotecan (HYCAMTIN), CPT-11 (irinotecan, CAMPTOSAR), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammalI and calicheamicin omegalI (see, e.g., Nicolaou et al., Angew. Chem Intl. Ed. Engl., 33:183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN, morpholino-doxorubicin, cyanomorpholino-doxorubicin, 2-pyrrolino-doxorubicin, doxorubicin HCl liposome injection (DOXIL) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR), tegafur (UFTORAL), capecitabine (XELODA), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE, FILDESIN); dacarbazine; mannomustine; mitobronitol; mitolactol; pipobroman; gacytosine; arabinoside (“Ara-C”); thiotepa; taxoids, e.g., paclitaxel (TAXOL), albumin-engineered nanoparticle formulation of paclitaxel (ABRAXANE), and doxetaxel (TAXOTERE); chloranbucil; 6-thioguanine; mercaptopurine; methotrexate; platinum analogs such as cisplatin and carboplatin; vinblastine (VELBAN); platinum; etoposide (VP-16); ifosfamide; mitoxantrone; vincristine (ONCOVIN); oxaliplatin; leucovovin; vinorelbine (NAVELBINE); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN) combined with 5-FU and leucovovin.

Chemotherapeutic agents also include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX tamoxifen), raloxifene (EVISTA), droloxifene, 4-hydroxytamoxifen, trioxifene, keoxifene, LY117018, onapristone, and toremifene (FARESTON); anti-progesterones; estrogen receptor down-regulators (ERDs); estrogen receptor antagonists such as fulvestrant (FASLODEX); agents that function to suppress or shut down the ovaries, for example, leutinizing hormone-releasing hormone (LHRH) agonists such as leuprolide acetate (LUPRON and ELIGARD), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4 (5)-imidazoles, aminoglutethimide, megestrol acetate (MEGASE), exemestane (AROMASIN), formestanie, fadrozole, vorozole (RIVISOR), letrozole (FEMARA), and anastrozole (ARIMIDEX). In addition, such definition of chemotherapeutic agents includes bisphosphonates such as clodronate (for example, BONEFOS or OSTAC), etidronate (DIDROCAL), NE-58095, zoledronic acid/zoledronate (ZOMETA), alendronate (FOSAMAX), pamidronate (AREDIA), tiludronate (SKELID), or risedronate (ACTONEL); as well as troxacitabine (a 1,3-dioxolane nucleoside cytosine analog); anti-sense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE vaccine and gene therapy vaccines, for example, ALLOVECTIN vaccine, LEUVECTIN vaccine, and VAXID vaccine; topoisomerase 1 inhibitor (e.g., LURTOTECAN); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

A cytokine is a protein released by one cell that act on another cell as intercellular mediators or have an autocrine effect on the cells producing the proteins. Examples of such cytokines include lymphokines, monokines; interleukins (“ILs”) such as IL-1, IL-1α, IL-2, IL-3, IL-4, IL-5, IL-6, IL-7, IL-8, IL-9, IL10, IL-11, IL-12, IL-13, IL-15, IL-17A-F, IL-18 to IL-29 (such as IL-23), IL-31, including PROLEUKIN rIL-2; a tumor-necrosis factor such as TNF-α or TNF-β, TGF-β1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”).

A chemokine is a soluble factor (e.g., cytokine) that has the ability to selectively induce chemotaxis and activation of leukocytes. Chemokines also trigger processes of angiogenesis, inflammation, wound healing, and tumorigenesis. Non-limiting examples of chemokines include IL-8, a human homolog of murine keratinocyte chemoattractant (KC).

A growth factor is a substance, such as a vitamin or hormone, that is required for the stimulation of growth in living cells. In some embodiments, the AFFIMER® polypeptide can be combined with a growth factor selected from the group consisting of: adrenomedullin (AM), angiopoietin (Ang), BMPs, BDNF, EGF, erythropoietin (EPO), FGF, GDNF, G-CSF, GM-CSF, GDF9, HGF, HDGF, IGF, migration-stimulating factor, myostatin (GDF-8), NGF, neurotrophins, PDGF, thrombopoietin, TGF-α, TGF-β, TNF-α, VEGF, P1GF, IL-1, IL-2, IL-3, IL-4, IL-5, IL-6, IL-7, IL-12, IL-15, and IL-18.

An enzyme is a substance produced by a living organism which acts as a catalyst to bring about a specific biochemical reaction. PD-L1 AFFIMER® polypeptides may be conjugated to a sialidase, for example, so that the sialidase will cleave sialic acid motifs from the surface of PD-L1+ cells. Targeted cleavage of sialic acid motifs on the surface of HER2+ breast cancer cells has been shown to increase sensitivity to NK cell-mediated killing and may have a similar effect on PD-L1+ cancer cells. (10.1073/pnas. 1608069113)

### D. Treatments

The term “dysfunctional” includes refractory or unresponsive to antigen recognition, specifically, impaired capacity to translate antigen recognition into down-stream T-cell effector functions, such as proliferation, cytokine production (e.g., IL-2) and/or target cell killing.

“Anergy” refers to the state of unresponsiveness to antigen stimulation resulting from incomplete or insufficient signals delivered through the T-cell receptor (e.g., increase in intracellular Ca⁺² in the absence of ras-activation). T cell anergy can also result upon stimulation with antigen in the absence of co-stimulation, resulting in the cell becoming refractory to subsequent activation by the antigen even in the context of costimulation. The unresponsive state can often be overridden by the presence of Interleukin-2. Anergic T-cells do not undergo clonal expansion and/or acquire effector functions.

“Exhaustion” refers to T cell exhaustion as a state of T cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T cells. Exhaustion prevents optimal control of infection and tumors.

“Enhancing T-cell function” means to induce, cause or stimulate a T-cell to have a sustained or amplified biological function, or renew or reactivate exhausted or inactive T-cells. Examples of enhancing T-cell function include: increased secretion of y-interferon from CD8+ T-cells, increased proliferation, increased antigen responsiveness (e.g., viral, pathogen, or tumor clearance) relative to such levels before the intervention. In some embodiments, the level of enhancement is as least 50%, alternatively 60%, 70%, 80%, 90%, 100%, 120%, 150%, 200%. The manner of measuring this enhancement is known to one of ordinary skill in the art.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

A cancer is physiological condition in mammals in which a population of cells are characterized by unregulated cell growth. Examples of cancer include but are not limited to, carcinoma, blastoma, sarcoma, and hematologic cancers such as lymphoma and leukemia.

A tumor (also referred to as a neoplasm) is any mass of tissue that results from excessive cell growth or proliferation, either benign (noncancerous) or malignant (cancerous) including pre-cancerous lesions. Tumor growth is generally uncontrolled and progressive, does not induce or inhibit the proliferation of normal cells. Tumor can affect a variety of cells, tissues or organs, including but not limited to selected from bladder, bone, brain, breast, cartilage, glial cells, esophagus, fallopian tube, gall bladder, heart, intestine, kidney, liver, lung, lymph node, neural tissue, ovary, pancreas, prostate, skeletal muscle, skin, spinal cord, spleen, stomach, testis, thymus, thyroid, trachea, urethra, ureter, urethra, uterus, vagina organ or tissue or the corresponding cells. Tumors include cancers, such as sarcoma, carcinoma, plasmacytoma or (malignant plasma cells). Tumors of the present disclosure, may include but are not limited to leukemias (e.g., acute leukemia, acute lymphoblastic leukemia, acute myeloid leukemia, acute myeloid leukemia, acute promyelocytic leukemia, acute myeloid-monocytic leukemia, acute monocytic leukemia, acute leukemia, chronic leukemia, chronic myeloid leukemia, chronic lymphocytic leukemia, polycythemia vera), lymphomas (Hodgkin's disease, non-Hodgkin's disease), primary macroglobulinemia disease, heavy chain disease, and solid tumors such as sarcomas cancer (e.g., fibrosarcoma, myxosarcoma, liposarcoma, chondrosarcoma, osteosarcoma, chordoma, endothelium sarcoma, lymphangiosarcoma, angiosarcoma, lymphangioendothelio sarcoma, synovioma vioma, mesothelioma, Ewing's tumor, leiomyosarcoma, rhabdomyosarcoma, colon carcinoma, pancreatic cancer, breast cancer, (including triple negative breast cancer), ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, carcinoma, bronchogenic carcinoma, medullary carcinoma, renal cell carcinoma, hepatoma, Nile duct carcinoma, choriocarcinoma, spermatogonia Tumor, embryonal carcinoma, Wilms' tumor, cervical cancer, uterine cancer, testicular cancer, lung carcinoma (including small cell lung carcinoma and non-small cell lung carcinoma or NSCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma), esophageal cancer, gallbladder, kidney cancer, multiple myeloma. Preferably, a “tumor” includes, but is not limited to: pancreatic cancer, liver cancer, lung cancer (including NSCLC), stomach cancer, esophageal cancer, head and neck squamous cell carcinoma, prostate cancer, colon cancer, breast cancer (including triple negative breast cancer), lymphoma, gallbladder cancer, renal cancer, leukemia, multiple myeloma, ovarian cancer, cervical cancer and glioma.

Metastasis refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

“Cancer cell” and “tumor cell” refers to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

A “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

“Progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

“Overall response rate” (ORR) refers to the sum of complete response (CR) rate and partial response (PR) rate.

“Overall survival” refers to the percentage of individuals in a group who are likely to be alive after a particular duration of time.

“Treatment” refers to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus, those in need of treatment include those already with the disorder; those prone to have the disorder; and those in whom the disorder is to be prevented. In the case of cancer or a tumor, a subject is successfully “treated” according to the methods of the present disclosure if the patient shows at least one of the following: an increased immune response, an increased anti-tumor response, increased cytolytic activity of immune cells, increased killing of tumor cells by immune cells, a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of at least one symptom associated with the specific cancer; reduced morbidity and mortality; improvement in quality of life; reduction in tumorigenicity; reduction in the number or frequency of cancer stem cells; or some combination of effects.

“Subject,” “individual,” and “patient,” used interchangeably herein, refer to any animal (e.g., a mammal), including, but not limited to, humans, non-human primates, canines, felines, and rodents.

“Agonist” and “agonistic” refer to agents that are capable of, directly or indirectly, substantially inducing, activating, promoting, increasing, or enhancing the biological activity of a target or target pathway. “Agonist” is used herein to include any agent that partially or fully induces, activates, promotes, increases, or enhances the activity of a protein or other target of interest.

“Antagonist” and “antagonistic” refer to or describe an agent that is capable of, directly or indirectly, partially or fully blocking, inhibiting, reducing, or neutralizing a biological activity of a target and/or pathway. The term “antagonist” is used herein to include any agent that partially or fully blocks, inhibits, reduces, or neutralizes the activity of a protein or other target of interest.

“Modulation” and “modulate” refer to a change or an alteration in a biological activity. Modulation includes, but is not limited to, stimulating an activity or inhibiting an activity. Modulation may be an increase in activity or a decrease in activity, a change in binding characteristics, or any other change in the biological, functional, or immunological properties associated with the activity of a protein, a pathway, a system, or other biological targets of interest.

An immune response includes responses from both the innate immune system and the adaptive immune system. It includes both cell-mediated and/or humoral immune responses. It includes both T-cell and B-cell responses, as well as responses from other cells of the immune system such as natural killer (NK) cells, monocytes, macrophages, etc.

“Pharmaceutically acceptable” refers to a substance approved or approvable by a regulatory agency of the Federal government or a state government or listed in the U.S. Pharmacopeia or other generally recognized pharmacopeia for use in animals, including humans.

“Pharmaceutically acceptable excipient” is an excipient, carrier or adjuvant that can be administered to a subject, together with at least one agent of the present disclosure, and which does not destroy the pharmacological activity thereof and is nontoxic when administered in doses sufficient to deliver a therapeutic effect. In general, those of skill in the art and the U.S. FDA consider a pharmaceutically acceptable excipient, carrier, or adjuvant to be an inactive ingredient of any formulation.

An “effective amount” (also referred to herein as a “therapeutically effective amount” is an amount of an agent, such as a PD-L1 AFFIMER® agent, effective to treat a disease or disorder in a subject such as, a mammal. In the case of cancer or a tumor, the therapeutically effective amount of an PD-L1 AFFIMER® agent has a therapeutic effect and as such can boost the immune response, boost the anti-tumor response, increase cytolytic activity of immune cells, increase killing of tumor cells by immune cells, reduce the number of tumor cells; decrease tumorigenicity, tumorigenic frequency or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent at least one of the symptoms associated with the cancer; reduce morbidity and mortality; improve quality of life; or a combination of such effects.

### E. Miscellaneous

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

As used herein, reference to “about” or “approximately” a value or parameter includes (and describes) embodiments that are directed to that value or parameter. For example, description referring to “about X” includes description of “X”.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The phrase “at least one” may be used interchangeably with “one or more.” It should be understood that “a” is not limited to one but rather means “at least one.”

### III. PD-L1 AFFIMER® Polypeptides

An AFFIMER® polypeptide is a scaffold based on a Stefin A polypeptide, meaning that it has a sequence which is derived from a Stefin A polypeptide, for example, a mammalian Stefin A polypeptide, for example, a human Stefin A polypeptide. Some aspects of the application provide AFFIMER® polypeptides that bind PD-L1 (also referred to as “PD-L1 AFFIMER® polypeptides”) in which at least one of the solvent accessible loops from the wild-type Stefin A protein having the ability to bind PD-L1, preferably selectively, and preferably with Kd of 10⁻⁶M or less.

In some embodiments, a PD-L1 AFFIMER® polypeptide is derived from the wild-type human Stefin A polypeptide having a backbone sequence and in which one or both of loop 2 [designated (Xaa)ₙ] and loop 4 [designated (Xaa)ₘ] are replaced with alternative loop sequences (Xaa)ₙ and (Xaa)ₘ, to have the general Formula (I)

FR1-(Xaa)n-FR2-(Xaa)m-FR3 (I)
wherein
FR1 is a polypeptide sequence comprising the amino acid sequence of MIPGGLSEAK PATPEIQEIV DKVKPQLEEK TGETYGKLEA VQYKTQVX (SEQ ID NO: 1) or a polypeptide sequence having at least 70% identity to the amino acid sequence of SEQ ID NO: 1, wherein X is V or D;
FR2 is a polypeptide sequence comprising the amino acid sequence of GTNYYIKVRA GDNKYMHLKV FKSL (SEQ ID NO: 2) or a polypeptide sequence having at least 70% identity to the amino acid sequence of SEQ ID NO: 2;
FR3 is a polypeptide sequence comprising the amino acid sequence of EDLVLTGYQV DKNKDDELTG F (SEQ ID NO: 3) or a polypeptide sequence having at least 70% identity to the amino acid sequence of SEQ ID NO: 3; and
Xaa, individually for each occurrence, is an amino acid residue, n and m are each, independently, an integer from 3 to 20.

In some embodiments, FR1 is a polypeptide sequence having at least 80%, 85%, 90%, 95% or even 98% homology with SEQ ID NO: 1. In some embodiments, FR1 is a polypeptide sequence having at least 80%, 85%, 90%, 95% or even 98% identity with SEQ ID NO: 1; In some embodiments, FR2 is a polypeptide sequence having at least 80%, 85%, 90%, 95% or even 98% homology with SEQ ID NO: 2. In some embodiments, FR2 is a polypeptide sequence having at least 80%, 85%, 90%, 95% or even 98% identity with SEQ ID NO: 2; In some embodiments, FR3 is a polypeptide sequence having at least 80%, 85%, 90%, 95% or even 98% homology with SEQ ID NO: 3. In some embodiments, FR3 is a polypeptide sequence having at least 80%, 85%, 90%, 95% or even 98% identity with SEQ ID NO: 3.

In some embodiments, the PD-L1 AFFIMER® polypeptide has an amino acid sequence represented in the general Formula (II):

(SEQ ID NO: 4)
MIPGGLSEAKPATPEIQEIVDKVKPQLEEKTGETYGKLEAVQYKTQVV-
(Xaa)ₙ-GTNYYIKVRAGDNKYMHLKVFKSL-(Xaa)ₘ-EDLVLTGYQV
DKNKDDELTGF.

In other embodiments, the PD-L1 AFFIMER® polypeptide has an amino acid sequence represented in the general Formula (III):

(SEQ ID NO: 5)
MIPGGLSEAKPATPEIQEIVDKVKPQLEEKTGETYGKLEAVQYKTQVD-
(Xaa)ₙ-GTNYYIKVRAGDNKYMHLKVFKSL-(Xaa)ₘ-EDLVLTGYQV
DKNKDDELTGF.

In some embodiments, n is 3 to 15, 3 to 12, 3 to 9, 3 to 7, 5 to 7, 5 to 9, 5 to 12, 5 to 15, 7 to 12 or 7 to 9.

In some embodiments, m is 3 to 15, 3 to 12, 3 to 9, 3 to 7, 5 to 7, 5 to 9, 5 to 12, 5 to 15, 7 to 12 or 7 to 9.

In some embodiments, Xaa, independently for each occurrence, is an amino acid that can be added to a polypeptide by recombinant expression in a prokaryotic or eukaryotic cell, and even more preferably one of the 20 naturally occurring amino acids.

In some embodiments of the above sequences and formulas, (Xaa)ₙ is an amino acid sequence selected from SEQ ID NOs: 6 to 259, or an amino acid sequence having at least 80%, 85%, 90%, 95% or even 98% homology with a sequence selected from SEQ ID NOs: 6 to 259. In some embodiments, (Xaa)ₙ is an amino acid sequence having at least 80%, 85%, 90%, 95% or even 98% identity with a sequence selected from SEQ ID NOs: 6 to 259.

TABLE 1
Loop 2 Sequences

| Loop 2 | SEQ ID NO: |
|---|---|
| VYHVRWNYL | 6 |
| ASFIDIGWH | 7 |
| PNQHFIVPY | 8 |
| FQTWDHAWT | 9 |
| FLDWKQHWR | 10 |
| LSEAVYIWA | 11 |
| RQYYEWSWV | 12 |
| PLPDPIDWV | 13 |
| QSHVEGEWL | 14 |
| DDVKNGFVQ | 15 |
| EVQQQENFQ | 16 |
| QLKFPPYVS | 17 |
| LFREEKYNY | 18 |
| SELVDPLHL | 19 |
| PYFWFQHAK | 20 |
| HIQWPPWVE | 21 |
| IVYTIWNYV | 22 |
| EAHQDLHEW | 23 |
| YRFGPPYVK | 24 |
| ANSKWHYAF | 25 |
| KRPHFLQFS | 26 |
| HRLHFPIYY | 27 |
| YWEHHHYGF | 28 |
| EEGTPEWAP | 29 |
| VRHPRWFFI | 30 |
| FVFLEVKWD | 31 |
| DVKPKWQWT | 32 |
| AAFSVHPSS | 33 |
| FTFVDIHWY | 34 |
| KYVVQASKV | 35 |
| AQDLSAFQI | 36 |
| KYVVQASKV | 37 |
| NQPQPPWIR | 38 |
| THLNNNAWT | 39 |
| KYETHFYYE | 40 |
| WSFAQVGWH | 41 |
| HHFAEVKWY | 42 |
| RVPLDSNKW | 43 |
| EIRPVWDWS | 44 |
| KRRHFPQWQ | 45 |
| GTRDEWHWV | 46 |
| IQHIWWDFV | 47 |
| VQHPQWHWK | 48 |
| RTKETKKSW | 49 |
| REYPHFQGY | 50 |
| VQHSLWYFQ | 51 |
| GVKQTWNWN | 52 |
| TRKHFPQYW | 53 |
| KYYAIFDYK | 54 |
| GRQVKWAWT | 55 |
| SHHASPSHI | 56 |
| GIREDWNWS | 57 |
| AWDWLPAYR | 58 |
| INQQQIKHS | 59 |
| AGWSLPPHL | 60 |
| GGRHRWNWS | 61 |
| ELDGDHTWL | 62 |
| KQLIIWHWT | 63 |
| REPALWHER | 64 |
| PQVQEPWWI | 65 |
| PTGTHQWAA | 66 |
| FRQHFPNES | 67 |
| QEYEDQEWA | 68 |
| IAHIHWKYF | 69 |
| DYRAIWHWW | 70 |
| NLKPDVQGS | 71 |
| STKNLWRWS | 72 |
| RVQGLWNFQ | 73 |
| YKVDNNNYQ | 74 |
| IEEPSAFWW | 75 |
| ASFPEISWY | 76 |
| IHEPEIWWY | 77 |
| DWGWGIPWV | 78 |
| RRWELWSFQ | 79 |
| INQHENFKQ | 80 |
| HDFTIRYPW | 81 |
| WDRPRWGWT | 82 |
| DQGWWENWA | 83 |
| IWHSEWSYT | 84 |
| RRKHFPQWP | 85 |
| KAYVIWSYK | 86 |
| FWQLHRYGF | 87 |
| IYHELWNFY | 88 |
| LIRHLWSWS | 89 |
| NDGKSLVLA | 90 |
| DVRGQWAWT | 91 |
| KRLHLPQYP | 92 |
| AVHAPQYAP | 93 |
| DERVAWHWV | 94 |
| LGWWFGINQ | 95 |
| NQEHERRFI | 96 |
| DIDDDLWS | 97 |
| QWGVDLVYT | 98 |
| KVYLVWAWT | 99 |
| TIHPQAKYW | 100 |
| FNQSRHGH | 101 |
| ELDDAVKYW | 102 |
| DWAIWRDKKV | 103 |
| TWGERLHYP | 104 |
| HTDVDWHEW | 105 |
| HHHRFAKWW | 106 |
| RKAAVVARH | 107 |
| DESFPLVHWY | 108 |
| DNAHPLVKNW | 109 |
| ETSENVIQW | 110 |
| FSDLEWEGQ | 111 |
| FTDIHWSTW | 112 |
| REHASVKFW | 113 |
| ISGVGLVPN | 114 |
| HTQPKALDW | 115 |
| IQDRLGGFI | 116 |
| RFYHQWHEL | 117 |
| DWVTRHHTLV | 118 |
| QSSDLAKLW | 119 |
| RFYHQWHEL | 120 |
| DWVTRHHTLV | 121 |
| HEVRKTYEF | 122 |
| VISIPLVWN | 123 |
| SWVFKPFYH | 124 |
| PEVSITRWQ | 125 |
| GNFAHERWQ | 126 |
| VIHIPHQWH | 127 |
| NLVYYQRDW | 128 |
| QVLWEIFEH | 129 |
| TRLHFPQYQ | 130 |
| YEPHHPYPW | 131 |
| TQAIPWREW | 132 |
| PEVDKAEWW | 133 |
| QRVHFPDWP | 134 |
| PQPEWNEWG | 135 |
| NQWEWPVDL | 136 |
| ERYHFPQFT | 137 |
| LYSESRDYK | 138 |
| FWFEDIQWL | 139 |
| AVYPTFKY | 140 |
| VLRWQWGYL | 141 |
| LWPLQPKNW | 142 |
| DNVRHGWLS | 143 |
| GLIQPPYAQ | 144 |
| RQVGVWAYE | 145 |
| DLHTLKSGV | 146 |
| SWYKLWSYS | 147 |
| DWPSHAKFW | 148 |
| AYQSPPYHF | 149 |
| YKNVGPWVN | 150 |
| SKRSAWHWT | 151 |
| VHITVEWE | 152 |
| RDYDPFPYT | 153 |
| KTLGVWHWS | 154 |
| REVGLWRFQ | 155 |
| WEEVNWRNW | 156 |
| RAQLRPWVA | 157 |
| YPWSWNGHY | 158 |
| PQIGAPYVK | 159 |
| WRTGQKWQV | 160 |
| IQQLYKQTW | 161 |
| IYYPIWDFH | 162 |
| DNVANIVPH | 163 |
| HVHWAWNYL | 164 |
| GPWY FGSAW | 165 |
| VLERPPYVK | 166 |
| FTFSSIGWW | 167 |
| ISASEIRWY | 168 |
| RIWGFWAFQ | 169 |
| NEAESSREL | 170 |
| VAGLKWHWE | 171 |
| YRNKQWYNE | 172 |
| WEDTWPVPE | 173 |
| YANIGPWLY | 174 |
| HQFQEIKWE | 175 |
| IDHQNHPTE | 176 |
| SVFQYTYKF | 177 |
| DWPSHAKFW | 178 |
| RQVTIWHFE | 179 |
| WRRWFPQWP | 180 |
| FWYPEIGWL | 181 |
| ISEDSWWPH | 182 |
| DHLPHAKYW | 183 |
| FKTDDARWI | 184 |
| LQGSRLPIE | 185 |
| KAYKVENYI | 186 |
| LKTNPSPSD | 187 |
| RQTDSVKWW | 188 |
| HRKHFHQFT | 189 |
| VVLFASGWE | 190 |
| YDNVKEYPW | 191 |
| IQDWTVGWE | 192 |
| PQENAPWVL | 193 |
| FQSTDSFWH | 194 |
| GVKSTFHWS | 195 |
| LRYHFPEFH | 196 |
| TENNSWWPI | 197 |
| LRHPVQDDD | 198 |
| EERRIWNWT | 199 |
| VGVGRDFPK | 200 |
| LLWTPPYVD | 201 |
| WKPQDSVNW | 202 |
| SSPLHWELWHWS | 203 |
| RDHELVNQW | 204 |
| YGPHHQTYA | 205 |
| QRAHFKQWY | 206 |
| IRHVIWSYS | 207 |
| YHGPPKEWL | 208 |
| SLRPHEQLY | 209 |
| VFLSEIHWY | 210 |
| RADVNVIWW | 211 |
| DTKTPWLWT | 212 |
| QWKKTQYFF | 213 |
| IAKEENHWR | 214 |
| WEYAGDYLE | 215 |
| THPEDAYSW | 216 |
| YAFITPVTH | 217 |
| KQHTKEWAP | 218 |
| IKIHHGSWE | 219 |
| RRVFFPEFP | 220 |
| FTEVKPFVE | 221 |
| KKYVIFFYE | 222 |
| WRVYFPDFK | 223 |
| PLTIPPEVE | 224 |
| NRWNIREFE | 225 |
| IQDVDWRLW | 226 |
| QRDHFPEFP | 227 |
| FPWDQENYY | 228 |
| TRRHFVQFT | 229 |
| DLHTPHYAP | 230 |
| YKHDDEYGE | 231 |
| YVRWHEYGF | 232 |
| VWTSDDAWQ | 233 |
| HSSSKPYIH | 234 |
| HVSNFIIPH | 235 |
| ARWHWFHHF | 236 |
| TKHSATFFH | 237 |
| LRYHFQHEP | 238 |
| RNSGVWEFE | 239 |
| WNPPSLPIS | 240 |
| RDGTFWDFW | 241 |
| VAGLKWHWE | 242 |
| RHKGLWQFE | 243 |
| LTIPPTAFS | 244 |
| LLYQAPFVQ | 245 |
| SRLHFPNFQ | 246 |
| VEWSESGYD | 247 |
| WKQITLPVD | 248 |
| WDSEALPID | 249 |
| GYLELWKWS | 250 |
| ERFHFPQFP | 251 |
| RREHFPRWP | 252 |
| LQLPPRPHD | 253 |
| IYYPIWGFQ | 254 |
| RDPDVEVES | 255 |
| ATDQDWRKW | 256 |
| AVRPIFKWAG | 257 |
| TRPHEPGWN | 258 |
| ISDVSVGWE | 259 |

In some embodiments of the above sequences and formulas, (Xaa)ₘ is an amino acid sequence selected from SEQ ID NOs: 260 to 513, or an amino acid sequence having at least 80%, 85%, 90%, 95% or even 98% homology with a sequence selected from SEQ ID NOs: 260 to 513. In some embodiments, (Xaa)ₘ is an amino acid sequence having at least 80%, 85%, 90%, 95% or even 98% identity with a sequence selected from SEQ ID NOs: 260 to 513.

TABLE 2
Loop 4 Sequences

| Loop 4 | SEQ ID NO: |
|---|---|
| SLEEPPVET | 260 |
| REGKHGRNK | 261 |
| HWNGGYHGH | 262 |
| FAAETQQYY | 263 |
| FNDKHNST | 264 |
| NSRWYGPLG | 265 |
| FESPTEDND | 266 |
| HIHLNQIYR | 267 |
| FFAIHRLGI | 268 |
| IDRHVYWLE | 269 |
| HIYEHIVYR | 270 |
| GWDDKPEWD | 271 |
| AWGEVVSNA | 272 |
| RSFGGEWGW | 273 |
| WQSLLDKNE | 274 |
| GWTSDNSNF | 275 |
| TPOTETEHE | 276 |
| LTEHNKKHQERHLDH | 277 |
| GWNAFSSPA | 278 |
| DNAIYRGPT | 279 |
| NIGPAKKQL | 280 |
| HFTLPTQHP | 281 |
| AWPEIDPPV | 282 |
| WSDVLYNDE | 283 |
| KKHGDEVTL | 284 |
| RKERHQFDI | 285 |
| FEGEDRDAV | 286 |
| EKYHNIWYV | 287 |
| RKPKDRQYT | 288 |
| YGQKDGQKE | 289 |
| IFAPLKPWR | 290 |
| YGQKDGQKE | 291 |
| NWNSPNLTW | 292 |
| FFHNGKIYR | 293 |
| PKRWAYNAY | 294 |
| RRFEHLPFY | 295 |
| KITGSTLTF | 296 |
| FRVRDYSEL | 297 |
| FDHESKPTA | 298 |
| GDHLNQYPN | 299 |
| FVKDDGRVD | 300 |
| SRLEPAGHQ | 301 |
| VQQRDVWIN | 302 |
| QKIHAIFYV | 303 |
| ILNAGGIRW | 304 |
| FKHPEENPV | 305 |
| FGLNDDEPV | 306 |
| NEPLPVTNI | 307 |
| FKEKVRNSI | 308 |
| FKQHHNISE | 309 |
| TTDPNWDVH | 310 |
| FEPKKLEPA | 311 |
| FKFQNESFA | 312 |
| WQHIPPDFW | 313 |
| INHAGQKSD | 314 |
| FEPSGLAAE | 315 |
| RQLHRIWYF | 316 |
| FKNSNSHLE | 317 |
| TFKVDTWKV | 318 |
| YKAYIQGWN | 319 |
| WASLESGON | 320 |
| NGLEDPTQD | 321 |
| HLWNQQIYR | 322 |
| HLAKGQWRE | 323 |
| FHAEGDIWD | 324 |
| IVVKRYWYV | 325 |
| FNGAGGPDS | 326 |
| GHHGDHEKW | 327 |
| ITWQLNQPR | 328 |
| LRVRHSAYR | 329 |
| KNYPKTGVT | 330 |
| HKADRQKER | 331 |
| WLYGVKRLP | 332 |
| WQEHTDSVE | 333 |
| HLFHGLWYI | 334 |
| EYHVTKRDQ | 335 |
| FKESSRNDE | 336 |
| IWENNLRIH | 337 |
| FTRHSEPWE | 338 |
| DLQPREVFQ | 339 |
| FYGEPRGKG | 340 |
| EWNRLSPLW | 341 |
| DNNGDGNWQ | 342 |
| FEGENHQID | 343 |
| RLWDDRVSG | 344 |
| FQPRTKATE | 345 |
| GNIEPITDL | 346 |
| WSEKVAVEP | 347 |
| FNHRPGKPD | 348 |
| AHLQYEWGG | 349 |
| NPWLHPQDE | 350 |
| ILWPTKPER | 351 |
| IVKRAVILW | 352 |
| FQKGSNKPE | 353 |
| WKNLLKPIH | 354 |
| ELLHGLWWV | 355 |
| WWKTISADF | 356 |
| PIFEIVREG | 357 |
| WHKPKDNII | 358 |
| APAAPPWEF | 359 |
| WENENREDT | 360 |
| WPFLVIEKY | 361 |
| RFRDGGHHV | 362 |
| WRNDTSSIY | 363 |
| WPADILTQH | 364 |
| EKKWGWIYI | 365 |
| IPAWQQIHV | 366 |
| WLEATPHTQ | 367 |
| NWNY PQQPF | 368 |
| WGSILTDGP | 369 |
| HFYRGILYR | 370 |
| DLRAPRNDN | 371 |
| PPFKHWHL | 372 |
| WNLNSDKSS | 373 |
| DLRAPRNDN | 374 |
| PPFKHWHL | 375 |
| WEGGWPLAQ | 376 |
| IFQEYYPHR | 377 |
| SGPEFFSRH | 378 |
| YETHGNIIT | 379 |
| YIARDFDWT | 380 |
| IFHEPKPPR | 381 |
| EPGRWRWPK | 382 |
| FHQWSPALH | 383 |
| EIEVNPYAN | 384 |
| QYHPPYERF | 385 |
| IPEWPKDAS | 386 |
| IRYGLRHER | 387 |
| NKENQDEED | 388 |
| IFWNEKLIR | 389 |
| KVYRTHHIF | 390 |
| NHDQVRWNL | 391 |
| HAYKGSIYR | 392 |
| RFREDVDIR | 393 |
| KVEYLDFIT | 394 |
| QLLYEDND | 395 |
| GWNYLEAIA | 396 |
| FALLWSAYG | 397 |
| GWQYIDLFH | 398 |
| GRTVEGGRW | 399 |
| YIKHHLWWI | 400 |
| FEGRHKDPE | 401 |
| FLPDKDDPF | 402 |
| RQIAGYIAI | 403 |
| YRTNRGHER | 404 |
| FWNAPDDRQ | 405 |
| IYQRHTQSR | 406 |
| WRGPGPYAI | 407 |
| FEQHAENIE | 408 |
| YIGYAGDAQ | 409 |
| VPIHDHLRN | 410 |
| GWNEEARPP | 411 |
| IKYPFARAE | 412 |
| GWNSNIGVL | 413 |
| AGHYEVHIG | 414 |
| DYQHWPYDE | 415 |
| FWGDLKT | 416 |
| GWNYEHKWP | 417 |
| RNFYTEPHE | 418 |
| WENFHKLEK | 419 |
| NWEGEPSGV | 420 |
| RQLLDKPRY | 421 |
| KAFANDYYK | 422 |
| GHESGQNLW | 423 |
| HDFTLKWKF | 424 |
| IYASQYDYR | 425 |
| KIGYLEFVL | 426 |
| NWNDHLSTY | 427 |
| FKHRQQLHYT | 428 |
| FHRTPGSTT | 429 |
| WPWLHSTEQ | 430 |
| NNKPPHNDL | 431 |
| FLPDKDDPF | 432 |
| GRTEVEGSF | 433 |
| PEADADDPK | 434 |
| RSRYPNYTA | 435 |
| NWFHQPWQR | 436 |
| WYQPRWKVE | 437 |
| FNYNKTEHT | 438 |
| GWHNVTLPP | 439 |
| FKWDNGISD | 440 |
| AVFRQRLGV | 441 |
| WYNRYYNDF | 442 |
| SNDYYETYL | 443 |
| IQWRDHKPR | 444 |
| KWYDPTIRP | 445 |
| RIGRSTNLW | 446 |
| NWDQPDTVL | 447 |
| FVENWQKRT | 448 |
| FNNHPLSPD | 449 |
| NHLAIHELP | 450 |
| NWFSDEAPY | 451 |
| AGVNRWKWI | 452 |
| FTASDTHPA | 453 |
| YIHYLKVNE | 454 |
| GWNDDHSVG | 455 |
| DWDGAAGDA | 456 |
| ELNNKDQND | 457 |
| FGQPVQGNT | 458 |
| YALHINRWP | 459 |
| PGEPHHILD | 460 |
| WERNASLSE | 461 |
| FFWKGNGYR | 462 |
| HLKLWKIPN | 463 |
| RINNSGLSR | 464 |
| PVKYKEILL | 465 |
| FLKILAEDD | 466 |
| RIGYLEFEI | 467 |
| HYYQDVLYR | 468 |
| FHGIWWARV | 469 |
| YAFITPVTH | 470 |
| WLNNFGKNF | 471 |
| WNHLYHYPE | 472 |
| LFWQKQFYR | 473 |
| EQPIEGDLW | 474 |
| SWNAPFPAG | 475 |
| FVNKGGSDD | 476 |
| DEVDEIPHE | 477 |
| GWQQVNDVK | 478 |
| INVPVTELL | 479 |
| VPSDWSKTI | 480 |
| GHELYALYA | 481 |
| IKEPFIKNV | 482 |
| DEPATPTYL | 483 |
| WNTLLEFGG | 484 |
| KWTNRANKP | 485 |
| SNQLIPQPI | 486 |
| FKFEIYKHT | 487 |
| NWNRPYNDL | 488 |
| NWEGNDLAT | 489 |
| NPINGQETW | 490 |
| IGNGQYWYR | 491 |
| DGQGALSLP | 492 |
| GKYKTNWYF | 493 |
| AWDNTAKHG | 494 |
| SQDYNGGQA | 495 |
| IYASQYDYR | 496 |
| SHOPYRATI | 497 |
| GWNLHEVAL | 498 |
| GWDLEYSQK | 499 |
| NLEHPTDET | 500 |
| RLKSNVNIA | 501 |
| HWDEYEYDE | 502 |
| RWWPWDAVG | 503 |
| YNLHHPHKD | 504 |
| PHLDYIWNW | 505 |
| DLLLRKEEV | 506 |
| NWEPAQHPV | 507 |
| RWSKDGATA | 508 |
| WYYNDNQSG | 509 |
| IPLNWQLQE | 510 |
| FHGPLDNYT | 511 |
| DFDHNFIAD | 512 |
| RSGPAHVRR | 513 |

In some embodiments, the PD-L1 AFFIMER® polypeptide has an amino acid sequence selected from SEQ ID NOs: 514 to 767. In some embodiments, the PD-L1 AFFIMER® polypeptide has an amino acid sequence having at least 70%, 75% 80%, 85%, 90%, 95% or even 98% identity with a sequence selected from SEQ ID NOs: 514 to 767.

TABLE 3
Exemplary PD-L1 AFFIMER® Polypeptide Sequences

| Clone # | Amino acid sequence | SEQ ID NO: |
|---|---|---|
| 1 | MIPGGLSEAKPATPEIQEIVDKVKPQLEEKTGETYGKLEAVQYKTQVVVYHVR | 514 |
| | WNYLGTNYYIKVRAGDNKYMHLKVFKSLSLEEPPVETEDLVLTGYQVDKNKDD | |
| | ELTGF | |
| 2 | MIPGGLSEAKPATPEIQEIVDKVKPQLEEKTGETYGKLEAVQYKTQVVASFID | 515 |
| | IGWHGTNYYIKVRAGDNKYMHLKVFKSLREGKHGRNKEDLVLTGYQVDKNKDD | |
| | ELTGF | |
| 3 | MIPGGLSEAKPATPEIQEIVDKVKPQLEEKTGETYGKLEAVQYKTQVVPNQHE | 516 |
| | IVPYGTNYYIKVRAGDNKYMHLKVFKSLHWNGGYHGHEDLVLTGYQVDKNKDD | |
| | ELTGF | |
| 4 | MIPGGLSEAKPATPEIQEIVDKVKPQLEEKTGETYGKLEAVQYKTQVVFQTWD | 517 |
| | HAWTGTNYYIKVRAGDNKYMHLKVFKSLFAAETQQYYEDLVLTGYQVDKNKDD | |
| | ELTGF | |
| 5 | MIPGGLSEAKPATPEIQEIVDKVKPQLEEKTGETYGKLEAVQYKTQVVFLDWK | 518 |
| | QHWRGTNYYIKVRAGDNKYMHLKVFKSLFNDKHNSTEDLVLTGYQVDKNKDDE | |
| | LTGF | |
| 6 | MIPGGLSEAKPATPEIQEIVDKVKPQLEEKTGETYGKLEAVQYKTQVVLSEAV | 519 |
| | YIWAGTNYYIKVRAGDNKYMHLKVFKSLNSRWYGPLGEDLVLTGYQVDKNKDD | |
| | ELTGF | |
| 7 | MIPGGLSEAKPATPEIQEIVDKVKPQLEEKTGETYGKLEAVQYKTQVVRQYYE | 520 |
| | WSWVGTNYYIKVRAGDNKYMHLKVFKSLFESPTEDNDEDLVLTGYQVDKNKDD | |
| | ELTGF | |
| 8 | MIPGGLSEAKPATPEIQEIVDKVKPQLEEKTGETYGKLEAVQYKTQVVPLPDP | 521 |
| | IDWVGTNYYIKVRAGDNKYMHLKVFKSLHIHLNQIYREDLVLTGYQVDKNKDD | |
| | ELTGE | |
| 9 | MIPGGLSEAKPATPEIQEIVDKVKPQLEEKTGETYGKLEAVQYKTQVVQSHVE | 522 |
| | GEWLGTNYYIKVRAGDNKYMHLKVEKSLEFAIHRLGIEDLVLTGYQVDKNKDD | |
| | ELTGF | |
| 10 | MIPGGLSEAKPATPEIQEIVDKVKPQLEEKTGETYGKLEAVQYKTQVVDDVKN | 523 |
| | GFVQGTNYYIKVRAGDNKYMHLKVFKSLIDRHVYWLEEDLVLTGYQVDKNKDD | |
| | ELTGE | |
| 11 | MIPGGLSEAKPATPEIQEIVDKVKPQLEEKTGETYGKLEAVQYKTQVVEVQQQ | 524 |
| | ENFQGTNYYIKVRAGDNKYMHLKVFKSLHIYEHIVYREDLVLTGYQVDKNKDD | |
| | ELTGF | |
| 12 | MIPGGLSEAKPATPEIQEIVDKVKPQLEEKTGETYGKLEAVQYKTQVVQLKFP | 525 |
| | PYVSGTNYYIKVRAGDNKYMHLKVFKSLGWDDKPEWDEDLVLTGYQVDKNKDD | |
| | ELTGF | |
| 13 | MIPGGLSEAKPATPEIQEIVDKVKPQLEEKTGETYGKLEAVQYKTQVVLFREE | 526 |
| | KYNYGTNYYIKVRAGDNKYMHLKVFKSLAWGEVVSNAEDLVLTGYQVDKNKDD | |
| | ELTGF | |
| 14 | MIPGGLSEAKPATPEIQEIVDKVKPQLEEKTGETYGKLEAVQYKTQVVSFLVD | 527 |
| | PLHLGTNYYIKVRAGDNKYMHLKVFKSLRSFGGEWGWEDLVLTGYQVDKNKDD | |
| | ELTGF | |
| 15 | MIPGGLSEAKPATPEIQEIVDKVKPQLEEKTGETYGKLEAVQYKTQVVPYFWE | 528 |
| | QHAKGTNYYIKVRAGDNKYMHLKVFKSLWQSLLDKNEEDLVLTGYQVDKNKDD | |
| | ELTGF | |
| 16 | MIPGGLSEAKPATPEIQEIVDKVKPQLEEKTGETYGKLEAVQYKTQVVHIQWP | 529 |
| | PWVEGTNYYIKVRAGDNKYMHLKVFKSLGWTSDNSNFEDLVLTGYQVDKNKDD | |
| | ELTGF | |
| 17 | MIPGGLSEAKPATPEIQEIVDKVKPQLEEKTGETYGKLEAVQYKTQVVIVYTI | 530 |
| | WNYVGTNYYIKVRAGDNKYMHLKVFKSLTPQTETEHEEDLVLTGYQVDKNKDD | |
| | ELTGF | |
| 18 | MIPGGLSEAKPATPEIQEIVDKVKPQLEEKTGETYGKLEAVQYKTQVVEAHQD | 531 |
| | LHFWGTNYYIKVRAGDNKYMHLKVFKSLLTEHNKKHQERHLDHEDLVLTGYQV | |
| | DKNKDDELTGF | |
| 19 | MIPGGLSEAKPATPEIQEIVDKVKPQLEEKTGETYGKLEAVQYKTQVVYRFGP | 532 |
| | PYVKGTNYYIKVRAGDNKYMHLKVFKSLGWNAFSSPAEDLVLTGYQVDKNKDD | |
| | ELTGF | |
| 20 | MIPGGLSEAKPATPEIQEIVDKVKPQLEEKTGETYGKLEAVQYKTQVVANSKW | 533 |
| | HYAFGTNYYIKVRAGDNKYMHLKVFKSLDNAIYRGPTEDLVLTGYQVDKNKDD | |
| | ELTGF | |
| 21 | MIPGGLSEAKPATPEIQEIVDKVKPQLEEKTGETYGKLEAVQYKTQVVKRPHF | 534 |
| | LQFSGTNYYIKVRAGDNKYMHLKVFKSLNIGPAKKQLEDLVLTGYQVDKNKDD | |
| | ELTGF | |
| 22 | MIPGGLSEAKPATPEIQEIVDKVKPQLEEKTGETYGKLEAVQYKTQVVHRLHF | 535 |
| | PIYYGTNYYIKVRAGDNKYMHLKVFKSLHFTLPTQHPEDLVLTGYQVDKNKDD | |
| | ELTGF | |
| 23 | MIPGGLSEAKPATPEIQEIVDKVKPQLEEKTGETYGKLEAVQYKTQVVYWEHH | 536 |
| | HYGFGTNYYIKVRAGDNKYMHLKVFKSLAWPEIDPPVEDLVLTGYQVDKNKDD | |
| | ELTGF | |
| 24 | MIPGGLSEAKPATPEIQEIVDKVKPQLEEKTGETYGKLEAVQYKTQVVEEGTP | 537 |
| | EWAPGTNYYIKVRAGDNKYMHLKVFKSLWSDVLYNDFEDLVLTGYQVDKNKDD | |
| | ELTGE | |
| 25 | MIPGGLSEAKPATPEIQEIVDKVKPQLEEKTGETYGKLEAVQYKTQVVVRHPR | 538 |
| | WFFIGTNYYIKVRAGDNKYMHLKVFKSLKKHGDEVTLEDLVLTGYQVDKNKDD | |
| | ELTGE | |
| 26 | MIPGGLSEAKPATPEIQEIVDKVKPQLEEKTGETYGKLEAVQYKTQVVFVELE | 539 |
| | VKWDGTNYYIKVRAGDNKYMHLKVFKSLRKERHQFDIEDLVLTGYQVDKNKDD | |
| | ELTGF | |
| 27 | MIPGGLSEAKPATPEIQEIVDKVKPQLEEKTGETYGKLEAVQYKTQVVDVKPK | 540 |
| | WQWTGTNYYIKVRAGDNKYMHLKVFKSLFEGEDRDAVEDLVLTGYQVDKNKDD | |
| | ELTGF | |
| 28 | MIPGGLSEAKPATPEIQEIVDKVKPQLEEKTGETYGKLEAVQYKTQVVAAFSV | 541 |
| | HPSSGTNYYIKVRAGDNKYMHLKVFKSLEKYHNIWYVEDLVLTGYQVDKNKDD | |
| | ELTGE | |
| 29 | MIPGGLSEAKPATPEIQEIVDKVKPQLEEKTGETYGKLEAVQYKTQVVFTFVD | 542 |
| | IHWYGTNYYIKVRAGDNKYMHLKVFKSLRKPKDRQYTEDLVLTGYQVDKNKDD | |
| | ELTGF | |
| 30 | MIPGGLSEAKPATPEIQEIVDKVKPQLEEKTGETYGKLEAVQYKTQVVKYVVQ | 543 |
| | ASKVGTNYYIKVRAGDNKYMHLKVFKSLYGQKDGQKEEDLVLTGYQVDKNKDD | |
| | ELTGE | |
| 31 | MIPGGLSEAKPATPEIQEIVDKVKPQLEEKTGETYGKLEAVQYKTQVVAQDLS | 544 |
| | AFQIGTNYYIKVRAGDNKYMHLKVFKSLIFAPLKPWREDLVLTGYQVDKNKDD | |
| | ELTGE | |
| 32 | MIPGGLSEAKPATPEIQEIVDKVKPQLEEKTGETYGKLEAVQYKTQVVKYVVQ | 545 |
| | ASKVGTNYYIKVRAGDNKYMHLKVFKSLYGQKDGQKEEDLVLTGYQVDKNKDD | |
| | ELTGF | |
| 33 | MIPGGLSEAKPATPEIQEIVDKVKPQLEEKTGETYGKLEAVQYKTQVVNQPQP | 546 |
| | PWIRGTNYYIKVRAGDNKYMHLKVFKSLNWNSPNLTWEDLVLTGYQVDKNKDD | |
| | ELTGE | |
| 34 | MIPGGLSEAKPATPEIQEIVDKVKPQLEEKTGETYGKLEAVQYKTQVVTHLNN | 547 |
| | NAWTGTNYYIKVRAGDNKYMHLKVFKSLFFHNGKIYREDLVLTGYQVDKNKDD | |
| | ELTGF | |
| 35 | MIPGGLSEAKPATPEIQEIVDKVKPQLEEKTGETYGKLEAVQYKTQVVKYETH | 548 |
| | FYYEGTNYYIKVRAGDNKYMHLKVFKSLPKRWAYNAYEDLVLTGYQVDKNKDD | |
| | ELTGF | |
| 36 | MIPGGLSEAKPATPEIQEIVDKVKPQLEEKTGETYGKLEAVQYKTQVVWSFAQ | 549 |
| | VGWHGTNYYIKVRAGDNKYMHLKVFKSLRRFEHLPFYEDLVLTGYQVDKNKDD | |
| | ELTGE | |
| 37 | MIPGGLSEAKPATPEIQEIVDKVKPQLEEKTGETYGKLEAVQYKTQVVHHFAE | 550 |
| | VKWYGTNYYIKVRAGDNKYMHLKVFKSLKITGSTLTFEDLVLTGYQVDKNKDD | |
| | ELTGF | |
| 38 | MIPGGLSEAKPATPEIQEIVDKVKPQLEEKTGETYGKLEAVQYKTQVVRVPLD | 551 |
| | SNKWGTNYYIKVRAGDNKYMHLKVEKSLERVRDYSFLEDLVLTGYQVDKNKDD | |
| | ELTGE | |
| 39 | MIPGGLSEAKPATPEIQEIVDKVKPQLEEKTGETYGKLEAVQYKTQVVEIRPV | 552 |
| | WDWSGTNYYIKVRAGDNKYMHLKVFKSLFDHESKPTAEDLVLTGYQVDKNKDD | |
| | ELTGF | |
| 40 | MIPGGLSEAKPATPEIQEIVDKVKPQLEEKTGETYGKLEAVQYKTQVVKRRHF | 553 |
| | PQWQGTNYYIKVRAGDNKYMHLKVFKSLGDHLNQYPNEDLVLTGYQVDKNKDD | |
| | ELTGF | |
| 41 | MIPGGLSEAKPATPEIQEIVDKVKPQLEEKTGETYGKLEAVQYKTQVVGTRDE | 554 |
| | WHWVGTNYYIKVRAGDNKYMHLKVFKSLFVKDDGRVDEDLVLTGYQVDKNKDD | |
| | ELTGF | |
| 42 | MIPGGLSEAKPATPEIQEIVDKVKPQLEEKTGETYGKLEAVQYKTQVVIQHIW | 555 |
| | WDFVGTNYYIKVRAGDNKYMHLKVFKSLSRLEPAGHQEDLVLTGYQVDKNKDD | |
| | ELTGF | |
| 43 | MIPGGLSEAKPATPEIQEIVDKVKPQLEEKTGETYGKLEAVQYKTQVVVQHPQ | 556 |
| | WHWKGTNYYIKVRAGDNKYMHLKVFKSLVQQRDVWINEDLVLTGYQVDKNKDD | |
| | ELTGE | |
| 44 | MIPGGLSEAKPATPEIQEIVDKVKPQLEEKTGETYGKLEAVQYKTQVVRTKET | 557 |
| | KKSWGTNYYIKVRAGDNKYMHLKVEKSLQKIHAIFYVEDLVLTGYQVDKNKDD | |
| | ELTGF | |
| 45 | MIPGGLSEAKPATPEIQEIVDKVKPQLEEKTGETYGKLEAVQYKTQVVREYPH | 558 |
| | FQGYGTNYYIKVRAGDNKYMHLKVEKSLILNAGGIRWEDLVLTGYQVDKNKDD | |
| | ELTGF | |
| 46 | MIPGGLSEAKPATPEIQEIVDKVKPQLEEKTGETYGKLEAVQYKTQVVVQHSL | 559 |
| | WYFQGTNYYIKVRAGDNKYMHLKVFKSLFKHPEENPVEDLVLTGYQVDKNKDD | |
| | ELTGE | |
| 47 | MIPGGLSEAKPATPEIQEIVDKVKPQLEEKTGETYGKLEAVQYKTQVVGVKQT | 560 |
| | WNWNGTNYYIKVRAGDNKYMHLKVFKSLFGLNDDEPVEDLVLTGYQVDKNKDD | |
| | ELTGF | |
| 48 | MIPGGLSEAKPATPEIQEIVDKVKPQLEEKTGETYGKLEAVQYKTQVVTRKHF | 561 |
| | PQYWGTNYYIKVRAGDNKYMHLKVFKSLNEPLPVTNIEDLVLTGYQVDKNKDD | |
| | ELTGF | |
| 49 | MIPGGLSEAKPATPEIQEIVDKVKPQLEEKTGETYGKLEAVQYKTQVVKYYAI | 562 |
| | FDYKGTNYYIKVRAGDNKYMHLKVFKSLFKEKVRNSIEDLVLTGYQVDKNKDD | |
| | ELTGE | |
| 50 | MIPGGLSEAKPATPEIQEIVDKVKPQLEEKTGETYGKLEAVQYKTQVVGRQVK | 563 |
| | WAWTGTNYYIKVRAGDNKYMHLKVFKSLFKQHHNISEEDLVLTGYQVDKNKDD | |
| | ELTGF | |
| 51 | MIPGGLSEAKPATPEIQEIVDKVKPQLEEKTGETYGKLEAVQYKTQVVSHHAS | 564 |
| | PSHIGTNYYIKVRAGDNKYMHLKVFKSLTTDPNWDVHEDLVLTGYQVDKNKDD | |
| | ELTGF | |
| 52 | MIPGGLSEAKPATPEIQEIVDKVKPQLEEKTGETYGKLEAVQYKTQVVGIRED | 565 |
| | WNWSGTNYYIKVRAGDNKYMHLKVFKSLFEPKKLEPAEDLVLTGYQVDKNKDD | |
| | ELTGF | |
| 53 | MIPGGLSEAKPATPEIQEIVDKVKPQLEEKTGETYGKLEAVQYKTQVVAWDWL | 566 |
| | PAYRGTNYYIKVRAGDNKYMHLKVEKSLFKFQNESFAEDLVLTGYQVDKNKDD | |
| | ELTGF | |
| 54 | MIPGGLSEAKPATPEIQEIVDKVKPQLEEKTGETYGKLEAVQYKTQVVINQQQ | 567 |
| | IKHSGTNYYIKVRAGDNKYMHLKVFKSLWQHIPPDFWEDLVLTGYQVDKNKDD | |
| | ELTGF | |
| 55 | MIPGGLSEAKPATPEIQEIVDKVKPQLEEKTGETYGKLEAVQYKTQVVAGWSL | 568 |
| | PPHLGTNYYIKVRAGDNKYMHLKVEKSLINHAGQKSDEDLVLTGYQVDKNKDD | |
| | ELTGF | |
| 56 | MIPGGLSEAKPATPEIQEIVDKVKPQLEEKTGETYGKLEAVQYKTQVVGGRHR | 569 |
| | WNWSGTNYYIKVRAGDNKYMHLKVFKSLFEPSGLAAEEDLVLTGYQVDKNKDD | |
| | ELTGF | |
| 57 | MIPGGLSEAKPATPEIQEIVDKVKPQLEEKTGETYGKLEAVQYKTQVVELDGD | 570 |
| | HTWLGTNYYIKVRAGDNKYMHLKVFKSLRQLHRIWYFEDLVLTGYQVDKNKDD | |
| | ELTGF | |
| 58 | MIPGGLSEAKPATPEIQEIVDKVKPQLEEKTGETYGKLEAVQYKTQVVKQLII | 571 |
| | WHWTGTNYYIKVRAGDNKYMHLKVEKSLFKNSNSHLEEDLVLTGYQVDKNKDD | |
| | ELTGE | |
| 59 | MIPGGLSEAKPATPEIQEIVDKVKPQLEEKTGETYGKLEAVQYKTQVVREPAL | 572 |
| | WHFRGTNYYIKVRAGDNKYMHLKVFKSLTFKVDTWKVEDLVLTGYQVDKNKDD | |
| | ELTGF | |
| 60 | MIPGGLSEAKPATPEIQEIVDKVKPQLEEKTGETYGKLEAVQYKTQVVPQVQE | 573 |
| | PWWIGTNYYIKVRAGDNKYMHLKVFKSLYKAYIQGWNEDLVLTGYQVDKNKDD | |
| | ELTGF | |
| 61 | MIPGGLSEAKPATPEIQEIVDKVKPQLEEKTGETYGKLEAVQYKTQVVPTGTH | 574 |
| | QWAAGTNYYIKVRAGDNKYMHLKVFKSLWASLESGQNEDLVLTGYQVDKNKDD | |
| | ELTGF | |
| 62 | MIPGGLSEAKPATPEIQEIVDKVKPQLEEKTGETYGKLEAVQYKTQVVFRQHE | 575 |
| | PNFSGTNYYIKVRAGDNKYMHLKVFKSINGLEDPTQDEDLVLTGYQVDKNKDD | |
| | ELTGF | |
| 63 | MIPGGLSEAKPATPEIQEIVDKVKPQLEEKTGETYGKLEAVQYKTQVVQEYED | 576 |
| | QEWAGTNYYIKVRAGDNKYMHLKVFKSLHLWNQQIYREDLVLTGYQVDKNKDD | |
| | ELTGF | |
| 64 | MIPGGLSEAKPATPEIQEIVDKVKPQLEEKTGETYGKLEAVQYKTQVVIAHIH | 577 |
| | WKYFGTNYYIKVRAGDNKYMHLKVFKSLHLAKGQWREEDLVLTGYQVDKNKDD | |
| | ELTGF | |
| 65 | MIPGGLSEAKPATPEIQEIVDKVKPQLEEKTGETYGKLEAVQYKTQVVDYRAI | 578 |
| | WHWWGTNYYIKVRAGDNKYMHLKVEKSLFHAEGDIWDEDLVLTGYQVDKNKDD | |
| | ELTGE | |
| 66 | MIPGGLSEAKPATPEIQEIVDKVKPQLEEKTGETYGKLEAVQYKTQVVNLKPD | 579 |
| | VQGSGTNYYIKVRAGDNKYMHLKVFKSLIVVKRYWYVEDLVLTGYQVDKNKDD | |
| | ELTGE | |
| 67 | MIPGGLSEAKPATPEIQEIVDKVKPQLEEKTGETYGKLEAVQYKTQVVSTKNL | 580 |
| | WRWSGTNYYIKVRAGDNKYMHLKVEKSLENGAGGPDSEDLVLTGYQVDKNKDD | |
| | ELTGE | |
| 68 | MIPGGLSEAKPATPEIQEIVDKVKPQLEEKTGETYGKLEAVQYKTQVVRVQGL | 581 |
| | WNFQGTNYYIKVRAGDNKYMHLKVFKSLGHHGDHEKWEDLVLTGYQVDKNKDD | |
| | ELTGE | |
| 69 | MIPGGLSEAKPATPEIQEIVDKVKPQLEEKTGETYGKLEAVQYKTQVVYKVDN | 582 |
| | NNYQGTNYYIKVRAGDNKYMHLKVFKSLITWQLNQPREDLVLTGYQVDKNKDD | |
| | ELTGE | |
| 70 | MIPGGLSEAKPATPEIQEIVDKVKPQLEEKTGETYGKLEAVQYKTQVVIEEPS | 583 |
| | AFWWGTNYYIKVRAGDNKYMHLKVFKSLLRVRHSAYREDLVLTGYQVDKNKDD | |
| | ELTGE | |
| 71 | MIPGGLSEAKPATPEIQEIVDKVKPQLEEKTGETYGKLEAVQYKTQVVASFPE | 584 |
| | ISWYGTNYYIKVRAGDNKYMHLKVFKSLKNYPKTGVTEDLVLTGYQVDKNKDD | |
| | ELTGF | |
| 72 | MIPGGLSEAKPATPEIQEIVDKVKPQLEEKTGETYGKLEAVQYKTQVVIHEPE | 585 |
| | IWWYGTNYYIKVRAGDNKYMHLKVFKSLHKADRQKEREDLVLTGYQVDKNKDD | |
| | ELTGF | |
| 73 | MIPGGLSEAKPATPEIQEIVDKVKPQLEEKTGETYGKLEAVQYKTQVVDWGWG | 586 |
| | IPWVGTNYYIKVRAGDNKYMHLKVFKSLWLYGVKRLPEDLVLTGYQVDKNKDD | |
| | ELTGF | |
| 74 | MIPGGLSEAKPATPEIQEIVDKVKPQLEEKTGETYGKLEAVQYKTQVVRRWEL | 587 |
| | WSFQGTNYYIKVRAGDNKYMHLKVFKSLWQEHTDSVEEDLVLTGYQVDKNKDD | |
| | ELTGF | |
| 75 | MIPGGLSEAKPATPEIQEIVDKVKPQLEEKTGETYGKLEAVQYKTQVVINQHE | 588 |
| | NFKQGTNYYIKVRAGDNKYMHLKVFKSLHLFHGLWYIEDLVLTGYQVDKNKDD | |
| | ELTGF | |
| 76 | MIPGGLSEAKPATPEIQEIVDKVKPQLEEKTGETYGKLEAVQYKTQVVHDFTI | 589 |
| | RYPWGTNYYIKVRAGDNKYMHLKVEKSLEYHVTKRDQEDLVLTGYQVDKNKDD | |
| | ELTGE | |
| 77 | MIPGGLSEAKPATPEIQEIVDKVKPQLEEKTGETYGKLEAVQYKTQVVWDRPR | 590 |
| | WGWTGTNYYIKVRAGDNKYMHLKVEKSLFKESSRNDEEDLVLTGYQVDKNKDD | |
| | ELTGE | |
| 78 | MIPGGLSEAKPATPEIQEIVDKVKPQLEEKTGETYGKLEAVQYKTQVVDQGWW | 591 |
| | ENWAGTNYYIKVRAGDNKYMHLKVFKSLIWENNLRIHEDLVLTGYQVDKNKDD | |
| | ELTGF | |
| 79 | MIPGGLSEAKPATPEIQEIVDKVKPQLEEKTGETYGKLEAVQYKTQVVIWHSE | 592 |
| | WSYTGTNYYIKVRAGDNKYMHLKVFKSLFTRHSEPWEEDLVLTGYQVDKNKDD | |
| | ELTGF | |
| 80 | MIPGGLSEAKPATPEIQEIVDKVKPQLEEKTGETYGKLEAVQYKTQVVRRKHF | 593 |
| | PQWPGTNYYIKVRAGDNKYMHLKVEKSLDLQPREVFQEDLVLTGYQVDKNKDD | |
| | ELTGE | |
| 81 | MIPGGLSEAKPATPEIQEIVDKVKPQLEEKTGETYGKLEAVQYKTQVVKAYVI | 594 |
| | WSYKGTNYYIKVRAGDNKYMHLKVEKSLFYGEPRGKGEDLVLTGYQVDKNKDD | |
| | ELTGF | |
| 82 | MIPGGLSEAKPATPEIQEIVDKVKPQLEEKTGETYGKLEAVQYKTQVVFWQLH | 595 |
| | RYGFGTNYYIKVRAGDNKYMHLKVFKSLEWNRLSPLWEDLVLTGYQVDKNKDD | |
| | ELTGF | |
| 83 | MIPGGLSEAKPATPEIQEIVDKVKPQLEEKTGETYGKLEAVQYKTQVVIYHEL | 596 |
| | WNFYGTNYYIKVRAGDNKYMHLKVFKSLDNNGDGNWQEDLVLTGYQVDKNKDD | |
| | ELTGF | |
| 84 | MIPGGLSEAKPATPEIQEIVDKVKPQLEEKTGETYGKLEAVQYKTQVVLIRHL | 597 |
| | WSWSGTNYYIKVRAGDNKYMHLKVFKSLFEGENHQIDEDLVLTGYQVDKNKDD | |
| | ELTGF | |
| 85 | MIPGGLSEAKPATPEIQEIVDKVKPQLEEKTGETYGKLEAVQYKTQVVNDGKS | 598 |
| | LVLAGTNYYIKVRAGDNKYMHLKVFKSLRLWDDRVSGEDLVLTGYQVDKNKDD | |
| | ELTGF | |
| 86 | MIPGGLSEAKPATPEIQEIVDKVKPQLEEKTGETYGKLEAVQYKTQVVDVRGQ | 599 |
| | WAWTGTNYYIKVRAGDNKYMHLKVFKSLFQPRTKATEEDLVLTGYQVDKNKDD | |
| | ELTGE | |
| 87 | MIPGGLSEAKPATPEIQEIVDKVKPQLEEKTGETYGKLEAVQYKTQVVKRLHL | 600 |
| | PQYPGTNYYIKVRAGDNKYMHLKVFKSLGNIEPITDLEDLVLTGYQVDKNKDD | |
| | ELTGF | |
| 88 | MIPGGLSEAKPATPEIQEIVDKVKPQLEEKTGETYGKLEAVQYKTQVVAVHAP | 601 |
| | QYAPGTNYYIKVRAGDNKYMHLKVFKSLWSEKVAVFPEDLVLTGYQVDKNKDD | |
| | ELTGF | |
| 89 | MIPGGLSEAKPATPEIQEIVDKVKPQLEEKTGETYGKLEAVQYKTQVVDERVA | 602 |
| | WHWVGTNYYIKVRAGDNKYMHLKVFKSLENHRPGKPDEDLVLTGYQVDKNKDD | |
| | ELTGF | |
| 90 | MIPGGLSEAKPATPEIQEIVDKVKPQLEEKTGETYGKLEAVQYKTQVVLGWWF | 603 |
| | GINQGTNYYIKVRAGDNKYMHLKVEKSLAHLQYEWGGEDLVLTGYQVDKNKDD | |
| | ELTGF | |
| 91 | MIPGGLSEAKPATPEIQEIVDKVKPQLEEKTGETYGKLEAVQYKTQVVNQEHF | 604 |
| | RRFIGTNYYIKVRAGDNKYMHLKVFKSLNPWLHPQDFEDLVLTGYQVDKNKDD | |
| | ELTGE | |
| 92 | MIPGGLSEAKPATPEIQEIVDKVKPQLEEKTGETYGKLEAVQYKTQVDIDDDL | 605 |
| | WSGTNYYIKVRAGDNKYMHLKVFKSLILWPTKPEREDLVLTGYQVDKNKDDEL | |
| | TGF | |
| 93 | MIPGGLSEAKPATPEIQEIVDKVKPQLEEKTGETYGKLEAVQYKTQVDQWGVD | 606 |
| | LVYTGTNYYIKVRAGDNKYMHLKVFKSLIVKRAVILWEDLVLTGYQVDKNKDD | |
| | ELTGF | |
| 94 | MIPGGLSEAKPATPEIQEIVDKVKPQLEEKTGETYGKLEAVQYKTQVDKVYLV | 607 |
| | WAWTGTNYYIKVRAGDNKYMHLKVFKSLFQKGSNKPEEDLVLTGYQVDKNKDD | |
| | ELTGF | |
| 95 | MIPGGLSEAKPATPEIQEIVDKVKPQLEEKTGETYGKLEAVQYKTQVDTIHPQ | 608 |
| | AKYWGTNYYIKVRAGDNKYMHLKVFKSLWKNLLKPIHEDLVLTGYQVDKNKDD | |
| | ELTGE | |
| 96 | MIPGGLSEAKPATPEIQEIVDKVKPQLEEKTGETYGKLEAVQYKTQVDENQSR | 609 |
| | HGHGTNYYIKVRAGDNKYMHLKVEKSLELLHGLWWVEDLVLTGYQVDKNKDDE | |
| | LTGF | |
| 97 | MIPGGLSEAKPATPEIQEIVDKVKPQLEEKTGETYGKLEAVQYKTQVDELDDA | 610 |
| | VKYWGTNYYIKVRAGDNKYMHLKVFKSLWWKTISADFEDLVLTGYQVDKNKDD | |
| | ELTGE | |
| 98 | MIPGGLSEAKPATPEIQEIVDKVKPQLEEKTGETYGKLEAVQYKTQVDWAIWR | 611 |
| | DKKVGTNYYIKVRAGDNKYMHLKVFKSLPIFEIVREGEDLVLTGYQVDKNKDD | |
| | ELTGE | |
| 99 | MIPGGLSEAKPATPEIQEIVDKVKPQLEEKTGETYGKLEAVQYKTQVDTWGER | 612 |
| | LHYPGTNYYIKVRAGDNKYMHLKVFKSLWHKPKDNIIEDLVLTGYQVDKNKDD | |
| | ELTGE | |
| 100 | MIPGGLSEAKPATPEIQEIVDKVKPQLEEKTGETYGKLEAVQYKTQVDHTDVD | 613 |
| | WHEWGTNYYIKVRAGDNKYMHLKVEKSLAPAAPPWEFEDLVLTGYQVDKNKDD | |
| | ELTGE | |
| 101 | MIPGGLSEAKPATPEIQEIVDKVKPQLEEKTGETYGKLEAVQYKTQVDHHHRF | 614 |
| | AKWWGTNYYIKVRAGDNKYMHLKVFKSLWFNENREDTEDLVLTGYQVDKNKDD | |
| | ELTGF | |
| 102 | MIPGGLSEAKPATPEIQEIVDKVKPQLEEKTGETYGKLEAVQYKTQVDRKAAV | 615 |
| | VARHGTNYYIKVRAGDNKYMHLKVFKSLWPFLVIEKYEDLVLTGYQVDKNKDD | |
| | ELTGE | |
| 103 | MIPGGLSEAKPATPEIQEIVDKVKPQLEEKTGETYGKLEAVQYKTQVDFSFPL | 616 |
| | VHWYGTNYYIKVRAGDNKYMHLKVFKSLRFRDGGHHVEDLVLTGYQVDKNKDD | |
| | ELTGF | |
| 104 | MIPGGLSEAKPATPEIQEIVDKVKPQLEEKTGETYGKLEAVQYKTQVDNAHPL | 617 |
| | VKNWGTNYYIKVRAGDNKYMHLKVFKSLWRNDTSSIYEDLVLTGYQVDKNKDD | |
| | ELTGF | |
| 105 | MIPGGLSEAKPATPEIQEIVDKVKPQLEEKTGETYGKLEAVQYKTQVDETSEN | 618 |
| | VIQWGTNYYIKVRAGDNKYMHLKVFKSLWPADILTQHEDLVLTGYQVDKNKDD | |
| | ELTGE | |
| 106 | MIPGGLSEAKPATPEIQEIVDKVKPQLEEKTGETYGKLEAVQYKTQVDESDLE | 619 |
| | WEGQGTNYYIKVRAGDNKYMHLKVEKSLEKKWGWIYIEDLVLTGYQVDKNKDD | |
| | ELTGF | |
| 107 | MIPGGLSEAKPATPEIQEIVDKVKPQLEEKTGETYGKLEAVQYKTQVDFTDIH | 620 |
| | WSTWGTNYYIKVRAGDNKYMHLKVFKSLIPAWQQIHVEDLVLTGYQVDKNKDD | |
| | ELTGE | |
| 108 | MIPGGLSEAKPATPEIQEIVDKVKPQLEEKTGETYGKLEAVQYKTQVDREHAS | 621 |
| | VKFWGTNYYIKVRAGDNKYMHLKVFKSLWLEATPHTQEDLVLTGYQVDKNKDD | |
| | ELTGE | |
| 109 | MIPGGLSEAKPATPEIQEIVDKVKPQLEEKTGETYGKLEAVQYKTQVDISGVG | 622 |
| | LVPNGTNYYIKVRAGDNKYMHLKVFKSLNWNYPQQPFEDLVLTGYQVDKNKDD | |
| | ELTGF | |
| 110 | MIPGGLSEAKPATPEIQEIVDKVKPQLEEKTGETYGKLEAVQYKTQVDHTQPK | 623 |
| | ALDWGTNYYIKVRAGDNKYMHLKVFKSLWGSILTDGPEDLVLTGYQVDKNKDD | |
| | ELTGF | |
| 111 | MIPGGLSEAKPATPEIQEIVDKVKPQLEEKTGETYGKLEAVQYKTQVDIQDRL | 624 |
| | GGFIGTNYYIKVRAGDNKYMHLKVFKSLHFYRGILYREDLVLTGYQVDKNKDD | |
| | ELTGE | |
| 112 | MIPGGLSEAKPATPEIQEIVDKVKPQLEEKTGETYGKLEAVQYKTQVDRFYHQ | 625 |
| | WHFLGTNYYIKVRAGDNKYMHLKVFKSLDLRAPRNDNEDLVLTGYQVDKNKDD | |
| | ELTGF | |
| 113 | MIPGGLSEAKPATPEIQEIVDKVKPQLEEKTGETYGKLEAVQYKTQVDWVTRH | 626 |
| | HTLVGTNYYIKVRAGDNKYMHLKVFKSLPPFKHWHLEDLVLTGYQVDKNKDDE | |
| | LTGF | |
| 114 | MIPGGLSEAKPATPEIQEIVDKVKPQLEEKTGETYGKLEAVQYKTQVDQSSDL | 627 |
| | AKLWGTNYYIKVRAGDNKYMHLKVFKSLWNLNSDKSSEDLVLTGYQVDKNKDD | |
| | ELTGF | |
| 115 | MIPGGLSEAKPATPEIQEIVDKVKPQLEEKTGETYGKLEAVQYKTQVDRFYHQ | 628 |
| | WHFLGTNYYIKVRAGDNKYMHLKVFKSLDLRAPRNDNEDLVLTGYQVDKNKDD | |
| | ELTGE | |
| 116 | MIPGGLSEAKPATPEIQEIVDKVKPQLEEKTGETYGKLEAVQYKTQVDWVTRH | 629 |
| | HTLVGTNYYIKVRAGDNKYMHLKVFKSLPPFKHWHLEDLVLTGYQVDKNKDDE | |
| | LTGF | |
| 117 | MIPGGLSEAKPATPEIQEIVDKVKPQLEEKTGETYGKLEAVQYKTQVDHEVRK | 630 |
| | TYEFGTNYYIKVRAGDNKYMHLKVFKSLWFGGWPLAQEDLVLTGYQVDKNKDD | |
| | ELTGE | |
| 118 | MIPGGLSEAKPATPEIQEIVDKVKPQLEEKTGETYGKLEAVQYKTQVDVISIP | 631 |
| | LVWNGTNYYIKVRAGDNKYMHLKVFKSLIFQEYYPHREDLVLTGYQVDKNKDD | |
| | ELTGE | |
| 119 | MIPGGLSEAKPATPEIQEIVDKVKPQLEEKTGETYGKLEAVQYKTQVDSWVFK | 632 |
| | PFYHGTNYYIKVRAGDNKYMHLKVFKSLSGPEFFSRHEDLVLTGYQVDKNKDD | |
| | ELTGF | |
| 120 | MIPGGLSEAKPATPEIQEIVDKVKPQLEEKTGETYGKLEAVQYKTQVDPEVSI | 633 |
| | TRWQGTNYYIKVRAGDNKYMHLKVFKSLYETHGNIITEDLVLTGYQVDKNKDD | |
| | ELTGF | |
| 121 | MIPGGLSEAKPATPEIQEIVDKVKPQLEEKTGETYGKLEAVQYKTQVDGNFAH | 634 |
| | ERWQGTNYYIKVRAGDNKYMHLKVFKSLYIARDFDWTEDLVLTGYQVDKNKDD | |
| | ELTGF | |
| 122 | MIPGGLSEAKPATPEIQEIVDKVKPQLEEKTGETYGKLEAVQYKTQVDVIHIP | 635 |
| | HQWHGTNYYIKVRAGDNKYMHLKVFKSLIFHEPKPPREDLVLTGYQVDKNKDD | |
| | ELTGF | |
| 123 | MIPGGLSEAKPATPEIQEIVDKVKPQLEEKTGETYGKLEAVQYKTQVDNLVYY | 636 |
| | QRDWGTNYYIKVRAGDNKYMHLKVEKSLEPGRWRWPKEDLVLTGYQVDKNKDD | |
| | ELTGF | |
| 124 | MIPGGLSEAKPATPEIQEIVDKVKPQLEEKTGETYGKLEAVQYKTQVDQVLWE | 637 |
| | IFEHGTNYYIKVRAGDNKYMHLKVFKSLFHQWSPALHEDLVLTGYQVDKNKDD | |
| | ELTGE | |
| 125 | MIPGGLSEAKPATPEIQEIVDKVKPQLEEKTGETYGKLEAVQYKTQVVTRLHF | 638 |
| | PQYQGTNYYIKVRAGDNKYMHLKVEKSLEIEVNPYANEDLVLTGYQVDKNKDD | |
| | ELTGE | |
| 126 | MIPGGLSEAKPATPEIQEIVDKVKPQLEEKTGETYGKLEAVQYKTQVVYEPHH | 639 |
| | PYPWGTNYYIKVRAGDNKYMHLKVFKSLQYHPPYERFEDLVLTGYQVDKNKDD | |
| | ELTGE | |
| 127 | MIPGGLSEAKPATPEIQEIVDKVKPQLEEKTGETYGKLEAVQYKTQVVTQAIP | 640 |
| | WREWGTNYYIKVRAGDNKYMHLKVFKSLIPEWPKDASEDLVLTGYQVDKNKDD | |
| | ELTGE | |
| 128 | MIPGGLSEAKPATPEIQEIVDKVKPQLEEKTGETYGKLEAVQYKTQVVPEVDK | 641 |
| | AEWWGTNYYIKVRAGDNKYMHLKVFKSLIRYGLRHEREDLVLTGYQVDKNKDD | |
| | ELTGF | |
| 129 | MIPGGLSEAKPATPEIQEIVDKVKPQLEEKTGETYGKLEAVQYKTQVVQRVHE | 642 |
| | PDWPGTNYYIKVRAGDNKYMHLKVFKSLNKENQDEEDEDLVLTGYQVDKNKDD | |
| | ELTGF | |
| 130 | MIPGGLSEAKPATPEIQEIVDKVKPQLEEKTGETYGKLEAVQYKTQVVPQPEW | 643 |
| | NEWGVTNYYIKVRAGDNKYMHLKVFKSLIFWNEKLIREDLVLTGYQVDKNKDD | |
| | ELTGF | |
| 131 | MIPGGLSEAKPATPEIQEIVDKVKPQLEEKTGETYGKLEAVQYKTQVVNQWEW | 644 |
| | PVDLGTNYYIKVRAGDNKYMHLKVFKSLKVYRTHHIFEDLVLTGYQVDKNKDD | |
| | ELTGE | |
| 132 | MIPGGLSEAKPATPEIQEIVDKVKPQLEEKTGETYGKLEAVQYKTQVVERYHF | 645 |
| | PQFTGTNYYIKVRAGDNKYMHLKVFKSLNHDQVRWNLEDLVLTGYQVDKNKDD | |
| | ELTGF | |
| 133 | MIPGGLSEAKPATPEIQEIVDKVKPQLEEKTGETYGKLEAVQYKTQVVLYSES | 646 |
| | RDYKGTNYYIKVRAGDNKYMHLKVFKSLHAYKGSIYREDLVLTGYQVDKNKDD | |
| | ELTGF | |
| 134 | MIPGGLSEAKPATPEIQEIVDKVKPQLEEKTGETYGKLEAVQYKTQVVFWFED | 647 |
| | IQWLGTNYYIKVRAGDNKYMHLKVFKSLRFREDVDIREDLVLTGYQVDKNKDD | |
| | ELTGE | |
| 135 | MIPGGLSEAKPATPEIQEIVDKVKPQLEEKTGETYGKLEAVQYKTQVVAVYPT | 648 |
| | FKYGTNYYIKVRAGDNKYMHLKVFKSLKVEYLDFITEDLVLTGYQVDKNKDDE | |
| | LTGF | |
| 136 | MIPGGLSEAKPATPEIQEIVDKVKPQLEEKTGETYGKLEAVQYKTQVVVLRWQ | 649 |
| | WGYLGTNYYIKVRAGDNKYMHLKVFKSLQLLYEDNDEDLVLTGYQVDKNKDDE | |
| | LTGF | |
| 137 | MIPGGLSEAKPATPEIQEIVDKVKPQLEEKTGETYGKLEAVQYKTQVVLWPLQ | 650 |
| | PKNWGTNYYIKVRAGDNKYMHLKVFKSLGWNYLEAIAEDLVLTGYQVDKNKDD | |
| | ELTGF | |
| 138 | MIPGGLSEAKPATPEIQEIVDKVKPQLEEKTGETYGKLEAVQYKTQVVDNVRH | 651 |
| | GWLSGTNYYIKVRAGDNKYMHLKVFKSLFALLWSAYGEDLVLTGYQVDKNKDD | |
| | ELTGF | |
| 139 | MIPGGLSEAKPATPEIQEIVDKVKPQLEEKTGETYGKLEAVQYKTQVVGLIQP | 652 |
| | PYAQGTNYYIKVRAGDNKYMHLKVFKSLGWQYIDLFHEDLVLTGYQVDKNKDD | |
| | ELTGE | |
| 140 | MIPGGLSEAKPATPEIQEIVDKVKPQLEEKTGETYGKLEAVQYKTQVVRQVGV | 653 |
| | WAYEGTNYYIKVRAGDNKYMHLKVFKSLGRTVEGGRWEDLVLTGYQVDKNKDD | |
| | ELTGE | |
| 141 | MIPGGLSEAKPATPEIQEIVDKVKPQLEEKTGETYGKLEAVQYKTQVVDLHTL | 654 |
| | KSGVGTNYYIKVRAGDNKYMHLKVEKSLYIKHHLWWIEDLVLTGYQVDKNKDD | |
| | ELTGE | |
| 142 | MIPGGLSEAKPATPEIQEIVDKVKPQLEEKTGETYGKLEAVQYKTQVVSWYKL | 655 |
| | WSYSGTNYYIKVRAGDNKYMHLKVFKSLFEGRHKDPEEDLVLTGYQVDKNKDD | |
| | ELTGF | |
| 143 | MIPGGLSEAKPATPEIQEIVDKVKPQLEEKTGETYGKLEAVQYKTQVVDWPSH | 656 |
| | AKFWGTNYYIKVRAGDNKYMHLKVFKSLFLPDKDDPFEDLVLTGYQVDKNKDD | |
| | ELTGE | |
| 144 | MIPGGLSEAKPATPEIQEIVDKVKPQLEEKTGETYGKLEAVQYKTQVVAYQSP | 657 |
| | PYHFGTNYYIKVRAGDNKYMHLKVFKSLRQIAGYIAIEDLVLTGYQVDKNKDD | |
| | ELTGF | |
| 145 | MIPGGLSEAKPATPEIQEIVDKVKPQLEEKTGETYGKLEAVQYKTQVVYKNVG | 658 |
| | PWVNGTNYYIKVRAGDNKYMHLKVFKSLYRTNRGHEREDLVLTGYQVDKNKDD | |
| | ELTGF | |
| 146 | MIPGGLSEAKPATPEIQEIVDKVKPQLEEKTGETYGKLEAVQYKTQVVSKRSA | 659 |
| | WHWTGTNYYIKVRAGDNKYMHLKVFKSLFWNAPDDRQEDLVLTGYQVDKNKDD | |
| | ELTGF | |
| 147 | MIPGGLSEAKPATPEIQEIVDKVKPQLEEKTGETYGKLEAVQYKTQVVVHITV | 660 |
| | EWEGTNYYIKVRAGDNKYMHLKVFKSLIYQRHTQSREDLVLTGYQVDKNKDDE | |
| | LTGF | |
| 148 | MIPGGLSEAKPATPEIQEIVDKVKPQLEEKTGETYGKLEAVQYKTQVVRDYDP | 661 |
| | FPYTGTNYYIKVRAGDNKYMHLKVFKSLWRGPGPYAIEDLVLTGYQVDKNKDD | |
| | ELTGE | |
| 149 | MIPGGLSEAKPATPEIQEIVDKVKPQLEEKTGETYGKLEAVQYKTQVVKTLGV | 662 |
| | WHWSGTNYYIKVRAGDNKYMHLKVFKSLFEQHAENIEEDLVLTGYQVDKNKDD | |
| | ELTGE | |
| 150 | MIPGGLSEAKPATPEIQEIVDKVKPQLEEKTGETYGKLEAVQYKTQVVREVGL | 663 |
| | WRFQGTNYYIKVRAGDNKYMHLKVFKSLYIGYAGDAQEDLVLTGYQVDKNKDD | |
| | ELTGF | |
| 151 | MIPGGLSEAKPATPEIQEIVDKVKPQLEEKTGETYGKLEAVQYKTQVVWEEVN | 664 |
| | WRNWGTNYYIKVRAGDNKYMHLKVFKSLVPIHDHLRNEDLVLTGYQVDKNKDD | |
| | ELTGF | |
| 152 | MIPGGLSEAKPATPEIQEIVDKVKPQLEEKTGETYGKLEAVQYKTQVVRAQLR | 665 |
| | PWVAGTNYYIKVRAGDNKYMHLKVFKSLGWNEEARPPEDLVLTGYQVDKNKDD | |
| | ELTGE | |
| 153 | MIPGGLSEAKPATPEIQEIVDKVKPQLEEKTGETYGKLEAVQYKTQVVYPWSW | 666 |
| | NGHYGTNYYIKVRAGDNKYMHLKVFKSLIKYPFARAEEDLVLTGYQVDKNKDD | |
| | ELTGF | |
| 154 | MIPGGLSEAKPATPEIQEIVDKVKPQLEEKTGETYGKLEAVQYKTQVVPQIGA | 667 |
| | PYVKGTNYYIKVRAGDNKYMHLKVFKSLGWNSNIGVLEDLVLTGYQVDKNKDD | |
| | ELTGF | |
| 155 | MIPGGLSEAKPATPEIQEIVDKVKPQLEEKTGETYGKLEAVQYKTQVVWRTGQ | 668 |
| | KWQVGTNYYIKVRAGDNKYMHLKVFKSLAGHYEVHIGEDLVLTGYQVDKNKDD | |
| | ELTGF | |
| 156 | MIPGGLSEAKPATPEIQEIVDKVKPQLEEKTGETYGKLEAVQYKTQVVIQQLY | 669 |
| | KQTWGTNYYIKVRAGDNKYMHLKVFKSLDYQHWPYDFEDLVLTGYQVDKNKDD | |
| | ELTGF | |
| 157 | MIPGGLSEAKPATPEIQEIVDKVKPQLEEKTGETYGKLEAVQYKTQVVIYYPI | 670 |
| | WDFHGTNYYIKVRAGDNKYMHLKVFKSLFWGDLKTEDLVLTGYQVDKNKDDEL | |
| | TGF | |
| 158 | MIPGGLSEAKPATPEIQEIVDKVKPQLEEKTGETYGKLEAVQYKTQVVDNVAN | 671 |
| | IVPHGTNYYIKVRAGDNKYMHLKVFKSLGWNYEHKWPEDLVLTGYQVDKNKDD | |
| | ELTGE | |
| 159 | MIPGGLSEAKPATPEIQEIVDKVKPQLEEKTGETYGKLEAVQYKTQVVHVHWA | 672 |
| | WNYLGTNYYIKVRAGDNKYMHLKVFKSLRNFYTFPHEEDLVLTGYQVDKNKDD | |
| | ELTGF | |
| 160 | MIPGGLSEAKPATPEIQEIVDKVKPQLEEKTGETYGKLEAVQYKTQVVGPWYF | 673 |
| | GSAWGTNYYIKVRAGDNKYMHLKVFKSLWENFHKLEKEDLVLTGYQVDKNKDD | |
| | ELTGF | |
| 161 | MIPGGLSEAKPATPEIQEIVDKVKPQLEEKTGETYGKLEAVQYKTQVVVLERP | 674 |
| | PYVKGTNYYIKVRAGDNKYMHLKVFKSLNWEGEPSGVEDLVLTGYQVDKNKDD | |
| | ELTGF | |
| 162 | MIPGGLSEAKPATPEIQEIVDKVKPQLEEKTGETYGKLEAVQYKTQVVFTFSS | 675 |
| | IGWWGTNYYIKVRAGDNKYMHLKVFKSLRQLLDKPRYEDLVLTGYQVDKNKDD | |
| | ELTGE | |
| 163 | MIPGGLSEAKPATPEIQEIVDKVKPQLEEKTGETYGKLEAVQYKTQVVISASE | 676 |
| | IRWYGTNYYIKVRAGDNKYMHLKVFKSLKAFANDYYKEDLVLTGYQVDKNKDD | |
| | ELTGE | |
| 164 | MIPGGLSEAKPATPEIQEIVDKVKPQLEEKTGETYGKLEAVQYKTQVVRIWGF | 677 |
| | WAFQGTNYYIKVRAGDNKYMHLKVFKSLGHESGQNLWEDLVLTGYQVDKNKDD | |
| | ELTGE | |
| 165 | MIPGGLSEAKPATPEIQEIVDKVKPQLEEKTGETYGKLEAVQYKTQVVNEAES | 678 |
| | SRFLGTNYYIKVRAGDNKYMHLKVFKSLHDFTLKWKFEDLVLTGYQVDKNKDD | |
| | ELTGF | |
| 166 | MIPGGLSEAKPATPEIQEIVDKVKPQLEEKTGETYGKLEAVQYKTQVVVAGLK | 679 |
| | WHWEGTNYYIKVRAGDNKYMHLKVEKSLIYASQYDYREDLVLTGYQVDKNKDD | |
| | ELTGE | |
| 167 | MIPGGLSEAKPATPEIQEIVDKVKPQLEEKTGETYGKLEAVQYKTQVVYRNKQ | 680 |
| | WYNEGTNYYIKVRAGDNKYMHLKVFKSLKIGYLEFVLEDLVLTGYQVDKNKDD | |
| | ELTGF | |
| 168 | MIPGGLSEAKPATPEIQEIVDKVKPQLEEKTGETYGKLEAVQYKTQVVWEDTW | 681 |
| | PVPEGTNYYIKVRAGDNKYMHLKVFKSLNWNDHLSTYEDLVLTGYQVDKNKDD | |
| | ELTGF | |
| 169 | MIPGGLSEAKPATPEIQEIVDKVKPQLEEKTGETYGKLEAVQYKTQVVYANIG | 682 |
| | PWLYGTNYYIKVRAGDNKYMHLKVFKSLFKHRQQLHYTEDLVLTGYQVDKNKD | |
| | DELTGF | |
| 170 | MIPGGLSEAKPATPEIQEIVDKVKPQLEEKTGETYGKLEAVQYKTQVVHQFQE | 683 |
| | IKWFGTNYYIKVRAGDNKYMHLKVFKSLFHRTPGSTTEDLVLTGYQVDKNKDD | |
| | ELTGE | |
| 171 | MIPGGLSEAKPATPEIQEIVDKVKPQLEEKTGETYGKLEAVQYKTQVVIDHQN | 684 |
| | HPTEGTNYYIKVRAGDNKYMHLKVFKSLWPWLHSTEQEDLVLTGYQVDKNKDD | |
| | ELTGF | |
| 172 | MIPGGLSEAKPATPEIQEIVDKVKPQLEEKTGETYGKLEAVQYKTQVVSVFQY | 685 |
| | TYKFGTNYYIKVRAGDNKYMHLKVFKSLNNKPPHNDLEDLVLTGYQVDKNKDD | |
| | ELTGF | |
| 173 | MIPGGLSEAKPATPEIQEIVDKVKPQLEEKTGETYGKLEAVQYKTQVVDWPSH | 686 |
| | AKFWGTNYYIKVRAGDNKYMHLKVFKSLFLPDKDDPFEDLVLTGYQVDKNKDD | |
| | ELTGF | |
| 174 | MIPGGLSEAKPATPEIQEIVDKVKPQLEEKTGETYGKLEAVQYKTQVVRQVTI | 687 |
| | WHFEGTNYYIKVRAGDNKYMHLKVFKSLGRTEVEGSFEDLVLTGYQVDKNKDD | |
| | ELTGF | |
| 175 | MIPGGLSEAKPATPEIQEIVDKVKPQLEEKTGETYGKLEAVQYKTQVVWRRWF | 688 |
| | PQWPGTNYYIKVRAGDNKYMHLKVFKSLPEADADDPKEDLVLTGYQVDKNKDD | |
| | ELTGE | |
| 176 | MIPGGLSEAKPATPEIQEIVDKVKPQLEEKTGETYGKLEAVQYKTQVVFWYPE | 689 |
| | IGWLGTNYYIKVRAGDNKYMHLKVFKSLRSRYPNYTAEDLVLTGYQVDKNKDD | |
| | ELTGF | |
| 177 | MIPGGLSEAKPATPEIQEIVDKVKPQLEEKTGETYGKLEAVQYKTQVVISEDS | 690 |
| | WWPHGTNYYIKVRAGDNKYMHLKVFKSLNWFHQPWQREDLVLTGYQVDKNKDD | |
| | ELTGF | |
| 178 | MIPGGLSEAKPATPEIQEIVDKVKPQLEEKTGETYGKLEAVQYKTQVVDHLPH | 691 |
| | AKYWGTNYYIKVRAGDNKYMHLKVFKSLWYQPRWKVEEDLVLTGYQVDKNKDD | |
| | ELTGE | |
| 179 | MIPGGLSEAKPATPEIQEIVDKVKPQLEEKTGETYGKLEAVQYKTQVVEKTDD | 692 |
| | ARWIGTNYYIKVRAGDNKYMHLKVEKSLENYNKTEHTEDLVLTGYQVDKNKDD | |
| | ELTGF | |
| 180 | MIPGGLSEAKPATPEIQEIVDKVKPQLEEKTGETYGKLEAVQYKTQVVLQGSR | 693 |
| | LPIEGTNYYIKVRAGDNKYMHLKVFKSLGWHNVTLPPEDLVLTGYQVDKNKDD | |
| | ELTGF | |
| 181 | MIPGGLSEAKPATPEIQEIVDKVKPQLEEKTGETYGKLEAVQYKTQVVKAYKV | 694 |
| | FNYIGTNYYIKVRAGDNKYMHLKVFKSLFKWDNGISDEDLVLTGYQVDKNKDD | |
| | ELTGF | |
| 182 | MIPGGLSEAKPATPEIQEIVDKVKPQLEEKTGETYGKLEAVQYKTQVVLKTNP | 695 |
| | SPSDGTNYYIKVRAGDNKYMHLKVFKSLAVFRQRLGVEDLVLTGYQVDKNKDD | |
| | ELTGF | |
| 183 | MIPGGLSEAKPATPEIQEIVDKVKPQLEEKTGETYGKLEAVQYKTQVVRQTDS | 696 |
| | VKWWGTNYYIKVRAGDNKYMHLKVFKSLWYNRYYNDFEDLVLTGYQVDKNKDD | |
| | ELTGF | |
| 184 | MIPGGLSEAKPATPEIQEIVDKVKPQLEEKTGETYGKLEAVQYKTQVVHRKHF | 697 |
| | HQFTGTNYYIKVRAGDNKYMHLKVFKSLSNDYYETYLEDLVLTGYQVDKNKDD | |
| | ELTGF | |
| 185 | MIPGGLSEAKPATPEIQEIVDKVKPQLEEKTGETYGKLEAVQYKTQVVVVLFA | 698 |
| | SGWEGTNYYIKVRAGDNKYMHLKVEKSLIQWRDHKPREDLVLTGYQVDKNKDD | |
| | ELTGF | |
| 186 | MIPGGLSEAKPATPEIQEIVDKVKPQLEEKTGETYGKLEAVQYKTQVVYDNVK | 699 |
| | EYPWGTNYYIKVRAGDNKYMHLKVEKSLKWYDPTIRPEDLVLTGYQVDKNKDD | |
| | ELTGF | |
| 187 | MIPGGLSEAKPATPEIQEIVDKVKPQLEEKTGETYGKLEAVQYKTQVVIQDWT | 700 |
| | VGWEGTNYYIKVRAGDNKYMHLKVFKSLRIGRSTNLWEDLVLTGYQVDKNKDD | |
| | ELTGE | |
| 188 | MIPGGLSEAKPATPEIQEIVDKVKPQLEEKTGETYGKLEAVQYKTQVVPQENA | 701 |
| | PWVLGTNYYIKVRAGDNKYMHLKVFKSLNWDQPDTVLEDLVLTGYQVDKNKDD | |
| | ELTGF | |
| 189 | MIPGGLSEAKPATPEIQEIVDKVKPQLEEKTGETYGKLEAVQYKTQVVFQSTD | 702 |
| | SFWHGTNYYIKVRAGDNKYMHLKVEKSLEVENWQKRTEDLVLTGYQVDKNKDD | |
| | ELTGF | |
| 190 | MIPGGLSEAKPATPEIQEIVDKVKPQLEEKTGETYGKLEAVQYKTQVVGVKST | 703 |
| | FHWSGTNYYIKVRAGDNKYMHLKVFKSLENNHPLSPDEDLVLTGYQVDKNKDD | |
| | ELTGF | |
| 191 | MIPGGLSEAKPATPEIQEIVDKVKPQLEEKTGETYGKLEAVQYKTQVVLRYHF | 704 |
| | PEFHGTNYYIKVRAGDNKYMHLKVFKSLNHLAIHELPEDLVLTGYQVDKNKDD | |
| | ELTGE | |
| 192 | MIPGGLSEAKPATPEIQEIVDKVKPQLEEKTGETYGKLEAVQYKTQVVTENNS | 705 |
| | WWPIGTNYYIKVRAGDNKYMHLKVFKSLNWFSDEAPYEDLVLTGYQVDKNKDD | |
| | ELTGF | |
| 193 | MIPGGLSEAKPATPEIQEIVDKVKPQLEEKTGETYGKLEAVQYKTQVVLRHPV | 706 |
| | QDDDGTNYYIKVRAGDNKYMHLKVFKSLAGVNRWKWIEDLVLTGYQVDKNKDD | |
| | ELTGF | |
| 194 | MIPGGLSEAKPATPEIQEIVDKVKPQLEEKTGETYGKLEAVQYKTQVVEERRI | 707 |
| | WNWTGTNYYIKVRAGDNKYMHLKVFKSLFTASDTHPAEDLVLTGYQVDKNKDD | |
| | ELTGF | |
| 195 | MIPGGLSEAKPATPEIQEIVDKVKPQLEEKTGETYGKLEAVQYKTQVVVGVGR | 708 |
| | DFPKGTNYYIKVRAGDNKYMHLKVFKSLYIHYLKVNEEDLVLTGYQVDKNKDD | |
| | ELTGF | |
| 196 | MIPGGLSEAKPATPEIQEIVDKVKPQLEEKTGETYGKLEAVQYKTQVVLLWTP | 709 |
| | PYVDGTNYYIKVRAGDNKYMHLKVFKSLGWNDDHSVGEDLVLTGYQVDKNKDD | |
| | ELTGF | |
| 197 | MIPGGLSEAKPATPEIQEIVDKVKPQLEEKTGETYGKLEAVQYKTQVVWKPQD | 710 |
| | SVNWGTNYYIKVRAGDNKYMHLKVFKSLDWDGAAGDAEDLVLTGYQVDKNKDD | |
| | ELTGF | |
| 198 | MIPGGLSEAKPATPEIQEIVDKVKPQLEEKTGETYGKLEAVQYKTQVVSSPLH | 711 |
| | WELWHWSGTNYYIKVRAGDNKYMHLKVEKSLELNNKDQNDEDLVLTGYQVDKN | |
| | KDDELTGE | |
| 199 | MIPGGLSEAKPATPEIQEIVDKVKPQLEEKTGETYGKLEAVQYKTQVVRDHEL | 712 |
| | VNQWGTNYYIKVRAGDNKYMHLKVFKSLFGQPVQGNTEDLVLTGYQVDKNKDD | |
| | ELTGF | |
| 200 | MIPGGLSEAKPATPEIQEIVDKVKPQLEEKTGETYGKLEAVQYKTQVVYGPHH | 713 |
| | QTYAGTNYYIKVRAGDNKYMHLKVFKSLYALHINRWPEDLVLTGYQVDKNKDD | |
| | ELTGF | |
| 201 | MIPGGLSEAKPATPEIQEIVDKVKPQLEEKTGETYGKLEAVQYKTQVVQRAHE | 714 |
| | KQWYGTNYYIKVRAGDNKYMHLKVFKSLPGEPHHILDEDLVLTGYQVDKNKDD | |
| | ELTGE | |
| 202 | MIPGGLSEAKPATPEIQEIVDKVKPQLEEKTGETYGKLEAVQYKTQVVIRHVI | 715 |
| | WSYSGTNYYIKVRAGDNKYMHLKVEKSLWERNASLSEEDLVLTGYQVDKNKDD | |
| | ELTGF | |
| 203 | MIPGGLSEAKPATPEIQEIVDKVKPQLEEKTGETYGKLEAVQYKTQVVYHGPP | 716 |
| | KEWLGTNYYIKVRAGDNKYMHLKVFKSLFFWKGNGYREDLVLTGYQVDKNKDD | |
| | ELTGF | |
| 204 | MIPGGLSEAKPATPEIQEIVDKVKPQLEEKTGETYGKLEAVQYKTQVVSLRPH | 717 |
| | EQLYGTNYYIKVRAGDNKYMHLKVFKSLHLKLWKIPNEDLVLTGYQVDKNKDD | |
| | ELTGE | |
| 205 | MIPGGLSEAKPATPEIQEIVDKVKPQLEEKTGETYGKLEAVQYKTQVVVELSE | 718 |
| | IHWYGTNYYIKVRAGDNKYMHLKVFKSLRINNSGLSREDLVLTGYQVDKNKDD | |
| | ELTGF | |
| 206 | MIPGGLSEAKPATPEIQEIVDKVKPQLEEKTGETYGKLEAVQYKTQVVRADVN | 719 |
| | VIWWGTNYYIKVRAGDNKYMHLKVFKSLPVKYKEILLEDLVLTGYQVDKNKDD | |
| | ELTGF | |
| 207 | MIPGGLSEAKPATPEIQEIVDKVKPQLEEKTGETYGKLEAVQYKTQVVDTKTP | 720 |
| | WLWTGTNYYIKVRAGDNKYMHLKVFKSLELKILAEDDEDLVLTGYQVDKNKDD | |
| | ELTGE | |
| 208 | MIPGGLSEAKPATPEIQEIVDKVKPQLEEKTGETYGKLEAVQYKTQVVQWKKT | 721 |
| | QYFFGTNYYIKVRAGDNKYMHLKVEKSLRIGYLEFEIEDLVLTGYQVDKNKDD | |
| | ELTGE | |
| 209 | MIPGGLSEAKPATPEIQEIVDKVKPQLEEKTGETYGKLEAVQYKTQVVIAKEE | 722 |
| | NHWRGTNYYIKVRAGDNKYMHLKVFKSLHYYQDVLYREDLVLTGYQVDKNKDD | |
| | ELTGE | |
| 210 | MIPGGLSEAKPATPEIQEIVDKVKPQLEEKTGETYGKLEAVQYKTQVVWEYAG | 723 |
| | DYLEGTNYYIKVRAGDNKYMHLKVFKSLFHGIWWARVEDLVLTGYQVDKNKDD | |
| | ELTGF | |
| 211 | MIPGGLSEAKPATPEIQEIVDKVKPQLEEKTGETYGKLEAVQYKTQVVTHPED | 724 |
| | AYSWGTNYYIKVRAGDNKYMHLKVFKSLLKPGGLLDFEDLVLTGYQVDKNKDD | |
| | ELTGE | |
| 212 | MIPGGLSEAKPATPEIQEIVDKVKPQLEEKTGETYGKLEAVQYKTQVVYAFIT | 725 |
| | PVTHGTNYYIKVRAGDNKYMHLKVFKSLWLNNFGKNFEDLVLTGYQVDKNKDD | |
| | ELTGF | |
| 213 | MIPGGLSEAKPATPEIQEIVDKVKPQLEEKTGETYGKLEAVQYKTQVVKQHTK | 726 |
| | EWAPGTNYYIKVRAGDNKYMHLKVFKSLWNHLYHYPFEDLVLTGYQVDKNKDD | |
| | ELTGF | |
| 214 | MIPGGLSEAEPATPEIQEIVDKVKPQLEEKTGETYGKLEAVQYKTQVVIKIHH | 727 |
| | GSWEGTNYYIKVRAGDNKYMHLKVFKSLLFWQKQFYREDLVLTGYQVDKNKDD | |
| | ELTGF | |
| 215 | MIPGGLSEAKPATPEIQEIVDKVKPQLEEKTGETYGKLEAVQYKTQVVRRVFF | 728 |
| | PEFPGTNYYIKVRAGDNKYMHLKVEKSLEQPIEGDLWEDLVLTGYQVDKNKDD | |
| | ELTGE | |
| 216 | MIPGGLSEAKPATPEIQEIVDKVKPQLEEKTGETYGKLEAVQYKTQVVFTEVK | 729 |
| | PFVEGTNYYIKVRAGDNKYMHLKVFKSLSWNAPFPAGEDLVLTGYQVDKNKDD | |
| | ELTGF | |
| 217 | MIPGGLSEAKPATPEIQEIVDKVKPQLEEKTGETYGKLEAVQYKTQVVKKYVI | 730 |
| | FFYEGTNYYIKVRAGDNKYMHLKVFKSLFVNKGGSDDEDLVLTGYQVDKNKDD | |
| | ELTGF | |
| 218 | MIPGGLSEAKPATPEIQEIVDKVKPQLEEKTGETYGKLEAVQYKTQVVWRVYF | 731 |
| | PDFKGTNYYIKVRAGDNKYMHLKVFKSLDEVDEIPHFEDLVLTGYQVDKNKDD | |
| | ELTGF | |
| 219 | MIPGGLSEAKPATPEIQEIVDKVKPQLEEKTGETYGKLEAVQYKTQVVPLTIP | 732 |
| | PFVEGTNYYIKVRAGDNKYMHLKVFKSLGWQQVNDVKEDLVLTGYQVDKNKDD | |
| | ELTGF | |
| 220 | MIPGGLSEAKPATPEIQEIVDKVKPQLEEKTGETYGKLEAVQYKTQVVNRWNI | 733 |
| | REFEGTNYYIKVRAGDNKYMHLKVFKSLINVPVTELLEDLVLTGYQVDKNKDD | |
| | ELTGE | |
| 221 | MIPGGLSEAKPATPEIQEIVDKVKPQLEEKTGETYGKLEAVQYKTQVVIQDVD | 734 |
| | WRLWGTNYYIKVRAGDNKYMHLKVFKSLVPSDWSKTIEDLVLTGYQVDKNKDD | |
| | ELTGF | |
| 222 | MIPGGLSEAEPATPEIQEIVDKVKPQLEEKTGETYGKLEAVQYKTQVVQRDHF | 735 |
| | PEFPGTNYYIKVRAGDNKYMHLKVFKSLGHELYALYAEDLVLTGYQVDKNKDD | |
| | ELTGF | |
| 223 | MIPGGLSEAKPATPEIQEIVDKVKPQLEEKTGETYGKLEAVQYKTQVVFPWDQ | 736 |
| | ENYYGTNYYIKVRAGDNKYMHLKVFKSLIKEPFIKNVEDLVLTGYQVDKNKDD | |
| | ELTGF | |
| 224 | MIPGGLSEAKPATPEIQEIVDKVKPQLEEKTGETYGKLEAVQYKTQVVTRRHE | 737 |
| | VQFTGTNYYIKVRAGDNKYMHLKVFKSLDEPATPTYLEDLVLTGYQVDKNKDD | |
| | ELTGF | |
| 225 | MIPGGLSEAKPATPEIQEIVDKVKPQLEEKTGETYGKLEAVQYKTQVVDLHTP | 738 |
| | HYAPGTNYYIKVRAGDNKYMHLKVFKSLWNTLLEFGGEDLVLTGYQVDKNKDD | |
| | ELTGF | |
| 226 | MIPGGLSEAKPATPEIQEIVDKVKPQLEEKTGETYGKLEAVQYKTQVVYKHDD | 739 |
| | EYGFGTNYYIKVRAGDNKYMHLKVFKSLKWTNRANKPEDLVLTGYQVDKNKDD | |
| | ELTGF | |
| 227 | MIPGGLSEAKPATPEIQEIVDKVKPQLEEKTGETYGKLEAVQYKTQVVYVRWH | 740 |
| | EYGFGTNYYIKVRAGDNKYMHLKVFKSLSNQLIPQPIEDLVLTGYQVDKNKDD | |
| | ELTGF | |
| 228 | MIPGGLSEAKPATPEIQEIVDKVKPQLEEKTGETYGKLEAVQYKTQVVVWTSD | 741 |
| | DAWQGTNYYIKVRAGDNKYMHLKVFKSLFKFEIYKHTEDLVLTGYQVDKNKDD | |
| | ELTGF | |
| 229 | MIPGGLSEAKPATPEIQEIVDKVKPQLEEKTGETYGKLEAVQYKTQVVHSSSK | 742 |
| | PYIHGTNYYIKVRAGDNKYMHLKVFKSLNWNRPYNDLEDLVLTGYQVDKNKDD | |
| | ELTGE | |
| 230 | MIPGGLSEAKPATPEIQEIVDKVKPQLEEKTGETYGKLEAVQYKTQVVHVSNF | 743 |
| | IIPHGTNYYIKVRAGDNKYMHLKVFKSLNWEGNDLATEDLVLTGYQVDKNKDD | |
| | ELTGE | |
| 231 | MIPGGLSEAKPATPEIQEIVDKVKPQLEEKTGETYGKLEAVQYKTQVVARWHW | 744 |
| | FHHFGTNYYIKVRAGDNKYMHLKVFKSLNPINGQETWEDLVLTGYQVDKNKDD | |
| | ELTGE | |
| 232 | MIPGGLSEAKPATPEIQEIVDKVKPQLEEKTGETYGKLEAVQYKTQVVTKHSA | 745 |
| | TFFHGTNYYIKVRAGDNKYMHLKVFKSLIGNGQYWYREDLVLTGYQVDKNKDD | |
| | ELTGF | |
| 233 | MIPGGLSEAKPATPEIQEIVDKVKPQLEEKTGETYGKLEAVQYKTQVVLRYHE | 746 |
| | QHFPGTNYYIKVRAGDNKYMHLKVFKSLDGQGALSLPEDLVLTGYQVDKNKDD | |
| | ELTGF | |
| 234 | MIPGGLSEAKPATPEIQEIVDKVKPQLEEKTGETYGKLEAVQYKTQVVRNSGV | 747 |
| | WEFEGTNYYIKVRAGDNKYMHLKVFKSLGKYKTNWYFEDLVLTGYQVDKNKDD | |
| | ELTGF | |
| 235 | MIPGGLSEAKPATPEIQEIVDKVKPQLEEKTGETYGKLEAVQYKTQVVWNPPS | 748 |
| | LPISGTNYYIKVRAGDNKYMHLKVFKSLAWDNTAKHGEDLVLTGYQVDKNKDD | |
| | ELTGF | |
| 236 | MIPGGLSEAKPATPEIQEIVDKVKPQLEEKTGETYGKLEAVQYKTQVVRDGTE | 749 |
| | WDFWGTNYYIKVRAGDNKYMHLKVFKSLSQDYNGGQAEDLVLTGYQVDKNKDD | |
| | ELTGE | |
| 237 | MIPGGLSEAKPATPEIQEIVDKVKPQLEEKTGETYGKLEAVQYKTQVVVAGLK | 750 |
| | WHWEGTNYYIKVRAGDNKYMHLKVEKSLIYASQYDYREDLVLTGYQVDKNKDD | |
| | ELTGF | |
| 238 | MIPGGLSEAKPATPEIQEIVDKVKPQLEEKTGETYGKLEAVQYKTQVVRHKGL | 751 |
| | WQFEGTNYYIKVRAGDNKYMHLKVFKSLSHQPYRATIEDLVLTGYQVDKNKDD | |
| | ELTGE | |
| 239 | MIPGGLSEAKPATPEIQEIVDKVKPQLEEKTGETYGKLEAVQYKTQVVLTIPP | 752 |
| | TAFSGTNYYIKVRAGDNKYMHLKVFKSLGWNLHEVALEDLVLTGYQVDKNKDD | |
| | ELTGF | |
| 240 | MIPGGLSEAKPATPEIQEIVDKVKPQLEEKTGETYGKLEAVQYKTQVVLLYQA | 753 |
| | PFVQGTNYYIKVRAGDNKYMHLKVEKSLGWDLEYSQKEDLVLTGYQVDKNKDD | |
| | ELTGF | |
| 241 | MIPGGLSEAKPATPEIQEIVDKVKPQLEEKTGETYGKLEAVQYKTQVVSRLHF | 754 |
| | PNFQGTNYYIKVRAGDNKYMHLKVFKSLNLEHPTDETEDLVLTGYQVDKNKDD | |
| | ELTGF | |
| 242 | MIPGGLSEAKPATPEIQEIVDKVKPQLEEKTGETYGKLEAVQYKTQVVVEWSE | 755 |
| | SGYDGTNYYIKVRAGDNKYMHLKVFKSLRLKSNVNIAEDLVLTGYQVDKNKDD | |
| | ELTGF | |
| 243 | MIPGGLSEAKPATPEIQEIVDKVKPQLEEKTGETYGKLEAVQYKTQVVWKQIT | 756 |
| | LPVDGTNYYIKVRAGDNKYMHLKVFKSLHWDEYEYDEEDLVLTGYQVDKNKDD | |
| | ELTGE | |
| 244 | MIPGGLSEAKPATPEIQEIVDKVKPQLEEKTGETYGKLEAVQYKTQVVWDSEA | 757 |
| | LPIDGTNYYIKVRAGDNKYMHLKVFKSLRWWPWDAVGEDLVLTGYQVDKNKDD | |
| | ELTGF | |
| 245 | MIPGGLSEAKPATPEIQEIVDKVKPQLEEKTGETYGKLEAVQYKTQVVGYLEL | 758 |
| | WKWSGTNYYIKVRAGDNKYMHLKVFKSLYNLHHPHKDEDLVLTGYQVDKNKDD | |
| | ELTGF | |
| 246 | MIPGGLSEAKPATPEIQEIVDKVKPQLEEKTGETYGKLEAVQYKTQVVERFHF | 759 |
| | PQFPGTNYYIKVRAGDNKYMHLKVEKSLPHLDYIWNWEDLVLTGYQVDKNKDD | |
| | ELTGE | |
| 247 | MIPGGLSEAKPATPEIQEIVDKVKPQLEEKTGETYGKLEAVQYKTQVVRREHE | 760 |
| | PRWPGTNYYIKVRAGDNKYMHLKVFKSLDLLLRKEEVEDLVLTGYQVDKNKDD | |
| | ELTGE | |
| 248 | MIPGGLSEAKPATPEIQEIVDKVKPQLEEKTGETYGKLEAVQYKTQVVLQLPP | 761 |
| | RPHDGTNYYIKVRAGDNKYMHLKVFKSLNWEPAQHPVEDLVLTGYQVDKNKDD | |
| | ELTGF | |
| 249 | MIPGGLSEAKPATPEIQEIVDKVKPQLEEKTGETYGKLEAVQYKTQVVIYYPI | 762 |
| | WGFQGTNYYIKVRAGDNKYMHLKVFKSLRWSKDGATAEDLVLTGYQVDKNKDD | |
| | ELTGF | |
| 250 | MIPGGLSEAKPATPEIQEIVDKVKPQLEEKTGETYGKLEAVQYKTQVVRDPDV | 763 |
| | FVFSGTNYYIKVRAGDNKYMHLKVEKSLWYYNDNQSGEDLVLTGYQVDKNKDD | |
| | ELTGF | |
| 251 | MIPGGLSEAKPATPEIQEIVDKVKPQLEEKTGETYGKLEAVQYKTQVVATDQD | 764 |
| | WRKWGTNYYIKVRAGDNKYMHLKVFKSLIPLNWQLQEEDLVLTGYQVDKNKDD | |
| | ELTGE | |
| 252 | MIPGGLSEAKPATPEIQEIVDKVKPQLEEKTGETYGKLEAVQYKTQVVAVRPI | 765 |
| | FKWAGTNYYIKVRAGDNKYMHLKVFKSLFHGPLDNYTEDLVLTGYQVDKNKDD | |
| | ELTGF | |
| 253 | MIPGGLSEAKPATPEIQEIVDKVKPQLEEKTGETYGKLEAVQYKTQVVTRPHF | 766 |
| | PGWNGTNYYIKVRAGDNKYMHLKVFKSLDEDHNFIADEDLVLTGYQVDKNKDD | |
| | ELTGE | |
| 254 | MIPGGLSEAKPATPEIQEIVDKVKPQLEEKTGETYGKLEAVQYKTQVVISDVS | 767 |
| | VGWEGTNYYIKVRAGDNKYMHLKVFKSLRSGPAHVRREDLVLTGYQVDKNKDD | |
| | ELTGE | |

In some embodiments, the PD-L1 AFFIMER® polypeptide has an amino acid sequence that is encoded by a nucleic acid having a coding sequence at least 70%, 75% 80%, 85%, 90%, 95% or even 98% identical with a sequence selected from SEQ ID NOs: 768 to 1021. In some embodiments, the PD-L1 AFFIMER® polypeptide has an amino acid sequence that is encoded by a nucleic acid that having a coding sequence that hybridizes to a sequence selected from SEQ ID NOs: 768 to 1021 under stringent conditions (such as in the presence of 6× sodium chloride/sodium citrate (SSC) at 45° C. followed by a wash in 0.2×SSC at 65° C.

TABLE 4
Exemplary Encoded PD-L1 AFFIMER ® Sequences

| Clone # | Nucleic Acid Sequences | SEQ ID NO: |
|---|---|---|
| 1 | ATGATCCCAGGTGGCCTGAGCGAAGCAAAACCAGCAACCCCAGAGATTCAAGA | 768 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTTACCATGTTCGT | |
| | TGGAACTACCTGGGTACGAACTACTACATCAAGGTTCGTGCGGGTGACAACAA | |
| | GTATATGCACCTGAAAGTGTTTAAGAGCCTGTCTCTGGAAGAACCACCAGTTG | |
| | AAACCGAAGATTTGGTGCTGACGGGCTACCAGGTTGACAAGAACAAAGATGAC | |
| | GAGCTGACGGGTTTC | |
| 2 | ATGATCCCAGGTGGCCTGAGCGAAGCAAAACCAGCAACCCCAGAGATTCAAGA | 769 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGCATCTTTCATCGAT | |
| | ATCGGTTGGCATGGTACGAACTACTACATCAAGGTTCGTGCGGGTGACAACAA | |
| | GTATATGCACCTGAAAGTGTTTAAGAGCCTGAGAGAAGGTAAACATGGTAGAA | |
| | ACAAAGAAGATTTGGTGCTGACGGGCTACCAGGTTGACAAGAACAAAGATGAC | |
| | GAGCTGACGGGTTTC | |
| 3 | ATGATCCCAGGTGGCCTGAGCGAAGCAAAACCAGCAACCCCAGAGATTCAAGA | 770 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCCAAACCAGCATTTC | |
| | ATCGTTCCATACGGTACGAACTACTACATCAAGGTTCGTGCGGGTGACAACAA | |
| | GTATATGCACCTGAAAGTGTTTAAGAGCCTGCATTGGAACGGTGGTTACCATG | |
| | GTCATGAAGATTTGGTGCTGACGGGCTACCAGGTTGACAAGAACAAAGATGAC | |
| | GAGCTGACGGGTTTC | |
| 4 | ATGATCCCAGGTGGCCTGAGCGAAGCAAAACCAGCAACCCCAGAGATTCAAGA | 771 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTTCCAGACCTGGGAT | |
| | CATGCATGGACCGGTACGAACTACTACATCAAGGTTCGTGCGGGTGACAACAA | |
| | GTATATGCACCTGAAAGTGTTTAAGAGCCTGTTCGCAGCAGAAACCCAGCAGT | |
| | ACTACGAAGATTTGGTGCTGACGGGCTACCAGGTTGACAAGAACAAAGATGAC | |
| | GAGCTGACGGGTTTC | |
| 5 | ATGATCCCAGGTGGCCTGAGCGAAGCAAAACCAGCAACCCCAGAGATTCAAGA | 772 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTTCCTGGATTGGAAA | |
| | CAGCATTGGCGTGGTACGAACTACTACATCAAGGTTCGTGCGGGTGACAACAA | |
| | GTATATGCACCTGAAAGTGTTTAAGAGCCTGTTCAACGATAAACATAACTCTA | |
| | CCGAAGATTTGGTGCTGACGGGCTACCAGGTTGACAAGAACAAAGATGACGAG | |
| | CTGACGGGTTTC | |
| 6 | ATGATCCCAGGTGGCCTGAGCGAAGCAAAACCAGCAACCCCAGAGATTCAAGA | 773 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGTCTGAAGCAGTT | |
| | TACATCTGGGCAGGTACGAACTACTACATCAAGGTTCGTGCGGGTGACAACAA | |
| | GTATATGCACCTGAAAGTGTTTAAGAGCCTGAACTCTAGATGGTACGGTCCAC | |
| | TGGGTGAAGATTTGGTGCTGACGGGCTACCAGGTTGACAAGAACAAAGATGAC | |
| | GAGCTGACGGGTTTC | |
| 7 | ATGATCCCAGGTGGCCTGAGCGAAGCAAAACCAGCAACCCCAGAGATTCAAGA | 774 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTCAGTACTACGAA | |
| | TGGTCTTGGGTTGGTACGAACTACTACATCAAGGTTCGTGCGGGTGACAACAA | |
| | GTATATGCACCTGAAAGTGTTTAAGAGCCTGTTCGAATCCCCAACCGAAGATA | |
| | ACGATGAAGATTTGGTGCTGACGGGCTACCAGGTTGACAAGAACAAAGATGAC | |
| | GAGCTGACGGGTTTC | |
| 8 | ATGATCCCAGGTGGCCTGAGCGAAGCAAAACCAGCAACCCCAGAGATTCAAGA | 775 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCCACTGCCAGATCCA | |
| | ATCGATTGGGTTGGTACGAACTACTACATCAAGGTTCGTGCGGGTGACAACAA | |
| | GTATATGCACCTGAAAGTGTTTAAGAGCCTGCATATCCATCTGAACCAGATCT | |
| | ACAGAGAAGATTTGGTGCTGACGGGCTACCAGGTTGACAAGAACAAAGATGAC | |
| | GAGCTGACGGGTTTC | |
| 9 | ATGATCCCAGGTGGCCTGAGCGAAGCAAAACCAGCAACCCCAGAGATTCAAGA | 776 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCAGTCTCATGTTGAA | |
| | GGTGAATGGCTGGGTACGAACTACTACATCAAGGTTCGTGCGGGTGACAACAA | |
| | GTATATGCACCTGAAAGTGTTTAAGAGCCTGTTCTTCGCAATCCATAGACTGG | |
| | GTATCGAAGATTTGGTGCTGACGGGCTACCAGGTTGACAAGAACAAAGATGAC | |
| | GAGCTGACGGGTTTC | |
| 10 | ATGATCCCAGGTGGCCTGAGCGAAGCAAAACCAGCAACCCCAGAGATTCAAGA | 777 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATGATGTTAAAAAC | |
| | GGTTTCGTTCAGGGTACGAACTACTACATCAAGGTTCGTGCGGGTGACAACAA | |
| | GTATATGCACCTGAAAGTGTTTAAGAGCCTGATCGATAGACATGTTTACTGGC | |
| | TGGAAGAAGATTTGGTGCTGACGGGCTACCAGGTTGACAAGAACAAAGATGAC | |
| | GAGCTGACGGGTTTC | |
| 11 | ATGATCCCAGGTGGCCTGAGCGAAGCAAAACCAGCAACCCCAGAGATTCAAGA | 778 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGAAGTTCAGCAGCAG | |
| | GAAAACTTCCAGGGTACGAACTACTACATCAAGGTTCGTGCGGGTGACAACAA | |
| | GTATATGCACCTGAAAGTGTTTAAGAGCCTGCATATCTACGAACATATCGTTT | |
| | ACAGAGAAGATTTGGTGCTGACGGGCTACCAGGTTGACAAGAACAAAGATGAC | |
| | GAGCTGACGGGTTTC | |
| 12 | ATGATCCCAGGTGGCCTGAGCGAAGCAAAACCAGCAACCCCAGAGATTCAAGA | 779 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCAGCTGAAATTCCCA | |
| | CCATACGTTTCTGGTACGAACTACTACATCAAGGTTCGTGCGGGTGACAACAA | |
| | GTATATGCACCTGAAAGTGTTTAAGAGCCTGGGTTGGGATGATAAACCAGAAT | |
| | GGGATGAAGATTTGGTGCTGACGGGCTACCAAGTTGACAAGAACAAAGATGAC | |
| | GAGCTGACGGGTTTC | |
| 13 | ATGATCCCAGGTGGCCTGAGCGAAGCAAAACCAGCAACCCCAGAGATTCAAGA | 780 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGTTCCGTGAAGAA | |
| | AAATACAACTACGGTACGAACTACTACATCAAGGTTCGTGCGGGTGACAACAA | |
| | GTATATGCACCTGAAAGTGTTTAAGAGCCTGGCATGGGGTGAAGTTGTTTCTA | |
| | ACGCAGAAGATTTGGTGCTGACGGGCTACCAGGTTGACAAGAACAAAGATGAC | |
| | GAGCTGACGGGTTTC | |
| 14 | ATGATCCCAGGTGGCCTGAGCGAAGCAAAACCAGCAACCCCAGAGATTCAAGA | 781 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTCTTTCCTGGTTGAT | |
| | CCACTGCATCTGGGTACGAACTACTACATCAAGGTTCGTGCGGGTGACAACAA | |
| | GTATATGCACCTGAAAGTGTTTAAGAGCCTGAGATCTTTCGGTGGTGAATGGG | |
| | GTTGGGAAGATTTGGTGCTGACGGGCTACCAGGTTGACAAGAACAAAGATGAC | |
| | GAGCTGACGGGTTTC | |
| 15 | ATGATCCCAGGTGGCCTGAGCGAAGCAAAACCAGCAACCCCAGAGATTCAAGA | 782 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCCATACTTCTGGTTC | |
| | CAGCATGCAAAAGGTACGAACTACTACATCAAGGTTCGTGCGGGTGACAACAA | |
| | GTATATGCACCTGAAAGTGTTTAAGAGCCTGTGGCAGTCTCTGCTGGATAAAA | |
| | ACGAAGAAGATTTGGTGCTGACGGGCTACCAGGTTGACAAGAACAAAGATGAC | |
| | GAGCTGACGGGTTTC | |
| 16 | ATGATCCCAGGTGGCCTGAGCGAAGCAAAACCAGCAACCCCAGAGATTCAAGA | 783 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCATATCCAGTGGCCA | |
| | CCATGGGTTGAAGGTACGAACTACTACATCAAGGTTCGTGCGGGTGACAACAA | |
| | GTATATGCACCTGAAAGTGTTTAAGAGCCTGGGTTGGACCTCTGATAACTCTA | |
| | ACTTCGAAGATTTGGTGCTGACGGGCTACCAGGTTGACAAGAACAAAGATGAC | |
| | GAGCTGACGGGTTTC | |
| 17 | ATGATCCCAGGTGGCCTGAGCGAAGCAAAACCAGCAACCCCAGAGATTCAAGA | 784 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCGTTTACACCATC | |
| | TGGAACTACGTTGGTACGAACTACTACATCAAGGTTCGTGCGGGTGACAACAA | |
| | GTATATGCACCTGAAAGTGTTTAAGAGCCTGACCCCACAGACCGAAACCGAAC | |
| | ATGAAGAAGATTTGGTGCTGACGGGCTACCAGGTTGACAAGAACAAAGATGAC | |
| | GAGCTGACGGGTTTC | |
| 18 | ATGATCCCAGGTGGCCTGAGCGAAGCAAAACCAGCAACCCCAGAGATTCAAGA | 785 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGAAGCACATCAGGAT | |
| | CTGCATTTCTGGGGTACGAACTACTACATCAAGGTTCGTGCGGGTGACAACAA | |
| | GTATATGCACCTGAAAGTGTTTAAGAGCCTGCTGACCGAACATAACAAAAAAC | |
| | ATCAGGAAAGACATCTGGATCATGAAGATTTGGTGCTGACGGGCTACCAGGTT | |
| | GACAAGAACAAAGATGACGAGCTGACGGGTTTC | |
| 19 | ATGATCCCAGGTGGCCTGAGCGAAGCAAAACCAGCAACCCCAGAGATTCAAGA | 786 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACCGTTTCGGTCCA | |
| | CCATACGTTAAAGGTACGAACTACTACATCAAGGTTCGTGCGGGTGACAACAA | |
| | GTATATGCACCTGAAAGTGTTTAAGAGCCTGGGTTGGAACGCATTCTCTTCTC | |
| | CAGCAGAAGATTTGGTGCTGACGGGCTACCAGGTTGACAAGAACAAAGATGAC | |
| | GAGCTGACGGGTTTC | |
| 20 | ATGATCCCAGGTGGCCTGAGCGAAGCAAAACCAGCAACCCCAGAGATTCAAGA | 787 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGCAAACTCTAAATGG | |
| | CATTACGCATTCGGTACGAACTACTACATCAAGGTTCGTGCGGGTGACAACAA | |
| | GTATATGCACCTGAAAGTGTTTAAGAGCCTGGATAACGCAATCTACAGAGGTC | |
| | CAACCGAAGATTTGGTGCTGACGGGCTACCAGGTTGACAAGAACAAAGATGAC | |
| | GAGCTGACGGGTTTC | |
| 21 | ATGATCCCAGGTGGCCTGAGCGAAGCAAAACCAGCAACCCCAGAGATTCAAGA | 788 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAAACGTCCACATTTC | |
| | CTGCAGTTCTCTGGTACGAACTACTACATCAAGGTTCGTGCGGGTGACAACAA | |
| | GTATATGCACCTGAAAGTGTTTAAGAGCCTGAACATCGGTCCAGCAAAAAAAC | |
| | AGCTGGAAGATTTGGTGCTGACGGGCTACCAGGTTGACAAGAACAAAGATGAC | |
| | GAGCTGACGGGTTTC | |
| 22 | ATGATCCCAGGTGGCCTGAGCGAAGCAAAACCAGCAACCCCAGAGATTCAAGA | 789 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCATCGTCTGCATTTC | |
| | CCAATCTACTACGGTACGAACTACTACATCAAGGTTCGTGCGGGTGACAACAA | |
| | GTATATGCACCTGAAAGTGTTTAAGAGCCTGCATTTCACCCTGCCAACCCAGC | |
| | ATCCAGAAGATTTGGTGCTGACGGGCTACCAGGTTGACAAGAACAAAGATGAC | |
| | GAGCTGACGGGTTTC | |
| 23 | ATGATCCCAGGTGGCCTGAGCGAAGCAAAACCAGCAACCCCAGAGATTCAAGA | 790 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACTGGGAACATCAT | |
| | CATTACGGTTTCGGTACGAACTACTACATCAAGGTTCGTGCGGGTGACAACAA | |
| | GTATATGCACCTGAAAGTGTTTAAGAGCCTGGCATGGCCAGAAATCGATCCAC | |
| | CAGTTGAAGATTTGGTGCTGACGGGCTACCAGGTTGACAAGAACAAAGATGAC | |
| | GAGCTGACGGGTTTC | |
| 24 | ATGATCCCAGGTGGCCTGAGCGAAGCAAAACCAGCAACCCCAGAGATTCAAGA | 791 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGAAGAAGGTACCCCA | |
| | GAATGGGCACCAGGTACGAACTACTACATCAAGGTTCGTGCGGGTGACAACAA | |
| | GTATATGCACCTGAAAGTGTTTAAGAGCCTGTGGTCTGATGTTCTGTACAACG | |
| | ATTTCGAAGATTTGGTGCTGACGGGCTACCAGGTTGACAAGAACAAAGATGAC | |
| | GAGCTGACGGGTTTC | |
| 25 | ATGATCCCAGGTGGCCTGAGCGAAGCAAAACCAGCAACCCCAGAGATTCAAGA | 792 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTCGTCATCCACGT | |
| | TGGTTCTTCATCGGTACGAACTACTACATCAAGGTTCGTGCGGGTGACAACAA | |
| | GTATATGCACCTGAAAGTGTTTAAGAGCCTGAAAAAACATGGTGATGAAGTTA | |
| | CCCTGGAAGATTTGGTGCTGACGGGCTACCAGGTTGACAAGAACAAAGATGAC | |
| | GAGCTGACGGGTTTC | |
| 26 | ATGATCCCAGGTGGCCTGAGCGAAGCAAAACCAGCAACCCCAGAGATTCAAGA | 793 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTTCGTTTTCCTGGAA | |
| | GTTAAATGGGATGGTACGAACTACTACATCAAGGTTCGTGCGGGTGACAACAA | |
| | GTATATGCACCTGAAAGTGTTTAAGAGCCTGAGAAAAGAAAGACATCAGTTCG | |
| | ATATCGAAGATTTGGTGCTGACGGGCTACCAGGTTGACAAGAACAAAGATGAC | |
| | GAGCTGACGGGTTTC | |
| 27 | ATGATCCCAGGTGGCCTGAGCGAAGCAAAACCAGCAACCCCAGAGATTCAAGA | 794 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATGTTAAACCAAAA | |
| | TGGCAGTGGACCGGTACGAACTACTACATCAAGGTTCGTGCGGGTGACAACAA | |
| | GTATATGCACCTGAAAGTGTTTAAGAGCCTGTTCGAAGGTGAAGATAGAGATG | |
| | CAGTTGAAGATTTGGTGCTGACGGGCTACCAAGTTGACAAGAACAAAGATGAC | |
| | GAGCTGACGGGTTTC | |
| 28 | ATGATCCCAGGTGGCCTGAGCGAAGCAAAACCAGCAACCCCAGAGATTCAAGA | 795 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGCAGCATTCTCTGTT | |
| | CATCCATCTTCTGGTACGAACTACTACATCAAGGTTCGTGCGGGTGACAACAA | |
| | GTATATGCACCTGAAAGTGTTTAAGAGCCTGGAAAAATACCATAACATCTGGT | |
| | ACGTTGAAGATTTGGTGCTGACGGGCTACCAGGTTGACAAGAACAAAGATGAC | |
| | GAGCTGACGGGTTTC | |
| 29 | ATGATCCCAGGTGGCCTGAGCGAAGCAAAACCAGCAACCCCAGAGATTCAAGA | 796 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTTCACCTTCGTTGAT | |
| | ATCCATTGGTACGGTACGAACTACTACATCAAGGTTCGTGCGGGTGACAACAA | |
| | GTATATGCACCTGAAAGTGTTTAAGAGCCTGAGAAAACCAAAAGATAGACAGT | |
| | ACACCGAAGATTTGGTGCTGACGGGCTACCAGGTTGACAAGAACAAAGATGAC | |
| | GAGCTGACGGGTTTC | |
| 30 | ATGATCCCAGGTGGCCTGAGCGAAGCAAAACCAGCAACCCCAGAGATTCAAGA | 797 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAAATACGTTGTTCAG | |
| | GCATCTAAAGTTGGTACGAACTACTACATCAAGGTTCGTGCGGGTGACAACAA | |
| | GTATATGCACCTGAAAGTGTTTAAGAGCCTGTACGGTCAGAAAGATGGTCAGA | |
| | AAGAAGAAGATTTGGTGCTGACGGGCTACCAGGTTGACAAGAACAAAGATGAC | |
| | GAGCTGACGGGTTTC | |
| 31 | ATGATCCCAGGTGGCCTGAGCGAAGCAAAACCAGCAACCCCAGAGATTCAAGA | 798 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGCACAGGATCTGTCT | |
| | GCATTCCAGATCGGTACGAACTACTACATCAAGGTTCGTGCGGGTGACAACAA | |
| | GTATATGCACCTGAAAGTGTTTAAGAGCCTGATCTTCGCACCACTGAAACCAT | |
| | GGAGAGAAGATTTGGTGCTGACGGGCTACCAGGTTGACAAGAACAAAGATGAC | |
| | GAGCTGACGGGTTTC | |
| 32 | ATGATCCCAGGTGGCCTGAGCGAAGCAAAACCAGCAACCCCAGAGATTCAAGA | 799 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAAATACGTTGTTCAG | |
| | GCATCTAAAGTTGGTACGAACTACTACATCAAGGTTCGTGCGGGTGACAACAA | |
| | GTATATGCACCTGAAAGTGTTTAAGAGCCTGTACGGTCAGAAAGATGGTCAGA | |
| | AAGAAGAAGATTTGGTGCTGACGGGCTACCAGGTTGACAAGAACAAAGATGAC | |
| | GAGCTGACGGGTTTC | |
| 33 | ATGATCCCAGGTGGCCTGAGCGAAGCAAAACCAGCAACCCCAGAGATTCAAGA | 800 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAACCAGCCACAGCCA | |
| | CCATGGATCCGTGGTACGAACTACTACATCAAGGTTCGTGCGGGTGACAACAA | |
| | GTATATGCACCTGAAAGTGTTTAAGAGCCTGAACTGGAACTCTCCAAACCTGA | |
| | CCTGGGAAGATTTGGTGCTGACGGGCTACCAGGTTGACAAGAACAAAGATGAC | |
| | GAGCTGACGGGTTTC | |
| 34 | ATGATCCCAGGTGGCCTGAGCGAAGCAAAACCAGCAACCCCAGAGATTCAAGA | 801 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCACCCATCTGAACAAC | |
| | AACGCATGGACCGGTACGAACTACTACATCAAGGTTCGTGCGGGTGACAACAA | |
| | GTATATGCACCTGAAAGTGTTTAAGAGCCTGTTCTTCCATAACGGTAAAATCT | |
| | ACAGAGAAGATTTGGTGCTGACGGGCTACCAGGTTGACAAGAACAAAGATGAC | |
| | GAGCTGACGGGTTTC | |
| 35 | ATGATCCCAGGTGGCCTGAGCGAAGCAAAACCAGCAACCCCAGAGATTCAAGA | 802 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAAATACGAAACCCAT | |
| | TTCTACTACGAAGGTACGAACTACTACATCAAGGTTCGTGCGGGTGACAACAA | |
| | GTATATGCACCTGAAAGTGTTTAAGAGCCTGCCAAAAAGATGGGCATACAACG | |
| | CATACGAAGATTTGGTGCTGACGGGCTACCAGGTTGACAAGAACAAAGATGAC | |
| | GAGCTGACGGGTTTC | |
| 36 | ATGATCCCAGGTGGCCTGAGCGAAGCAAAACCAGCAACCCCAGAGATTCAAGA | 803 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTGGTCTTTCGCACAG | |
| | GTTGGTTGGCATGGTACGAACTACTACATCAAGGTTCGTGCGGGTGACAACAA | |
| | GTATATGCACCTGAAAGTGTTTAAGAGCCTGAGAAGATTCGAACATCTGCCAT | |
| | TCTACGAAGATTTGGTGCTGACGGGCTACCAGGTTGACAAGAACAAAGATGAC | |
| | GAGCTGACGGGTTTC | |
| 37 | ATGATCCCAGGTGGCCTGAGCGAAGCAAAACCAGCAACCCCAGAGATTCAAGA | 804 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCATCATTTCGCAGAA | |
| | GTTAAATGGTACGGTACGAACTACTACATCAAGGTTCGTGCGGGTGACAACAA | |
| | GTATATGCACCTGAAAGTGTTTAAGAGCCTGAAAATCACCGGTTCTACCCTGA | |
| | CCTTCGAAGATTTGGTGCTGACGGGCTACCAGGTTGACAAGAACAAAGATGAC | |
| | GAGCTGACGGGTTTC | |
| 38 | ATGATCCCAGGTGGCCTGAGCGAAGCAAAACCAGCAACCCCAGAGATTCAAGA | 805 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTGTTCCACTGGAT | |
| | TCTAACAAATGGGGTACGAACTACTACATCAAGGTTCGTGCGGGTGACAACAA | |
| | GTATATGCACCTGAAAGTGTTTAAGAGCCTGTTCAGAGTTAGAGATTACTCTT | |
| | TCCTGGAAGATTTGGTGCTGACGGGCTACCAGGTTGACAAGAACAAAGATGAC | |
| | GAGCTGACGGGTTTC | |
| 39 | ATGATCCCAGGTGGCCTGAGCGAAGCAAAACCAGCAACCCCAGAGATTCAAGA | 806 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGAAATCCGTCCAGTT | |
| | TGGGATTGGTCTGGTACGAACTACTACATCAAGGTTCGTGCGGGTGACAACAA | |
| | GTATATGCACCTGAAAGTGTTTAAGAGCCTGTTCGATCATGAATCTAAACCAA | |
| | CCGCAGAAGATTTGGTGCTGACGGGCTACCAGGTTGACAAGAACAAAGATGAC | |
| | GAGCTGACGGGTTTC | |
| 40 | ATGATCCCAGGTGGCCTGAGCGAAGCAAAACCAGCAACCCCAGAGATTCAAGA | 807 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAAACGTCGTCATTTC | |
| | CCACAGTGGCAGGGTACGAACTACTACATCAAGGTTCGTGCGGGTGACAACAA | |
| | GTATATGCACCTGAAAGTGTTTAAGAGCCTGGGTGATCATCTGAACCAGTACC | |
| | CAAACGAAGATTTGGTGCTGACGGGCTACCAGGTTGACAAGAACAAAGATGAC | |
| | GAGCTGACGGGTTTC | |
| 41 | ATGATCCCAGGTGGCCTGAGCGAAGCAAAACCAGCAACCCCAGAGATTCAAGA | 808 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGGTACCCGTGATGAA | |
| | TGGCACTGGGTTGGTACGAACTACTACATCAAGGTTCGTGCGGGTGACAACAA | |
| | GTATATGCACCTGAAAGTGTTTAAGAGCCTGTTCGTTAAAGATGATGGTAGAG | |
| | TTGATGAAGATTTGGTGCTGACGGGCTACCAGGTTGACAAGAACAAAGATGAC | |
| | GAGCTGACGGGTTTC | |
| 42 | ATGATCCCAGGTGGCCTGAGCGAAGCAAAACCAGCAACCCCAGAGATTCAAGA | 809 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCCAGCATATCTGG | |
| | TGGGATTTCGTTGGTACGAACTACTACATCAAGGTTCGTGCGGGTGACAACAA | |
| | GTATATGCACCTGAAAGTGTTTAAGAGCCTGTCTAGACTGGAACCAGCAGGTC | |
| | ATCAGGAAGATTTGGTGCTGACGGGCTACCAGGTTGACAAGAACAAAGATGAC | |
| | GAGCTGACGGGTTTC | |
| 43 | ATGATCCCAGGTGGCCTGAGCGAAGCAAAACCAGCAACCCCAGAGATTCAAGA | 810 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTCAGCATCCACAG | |
| | TGGCATTGGAAAGGTACGAACTACTACATCAAGGTTCGTGCGGGTGACAACAA | |
| | GTATATGCACCTGAAAGTGTTTAAGAGCCTGGTTCAGCAGAGAGATGTTTGGA | |
| | TCAACGAAGATTTGGTGCTGACGGGCTACCAGGTTGACAAGAACAAAGATGAC | |
| | GAGCTGACGGGTTTC | |
| 44 | ATGATCCCAGGTGGCCTGAGCGAAGCAAAACCAGCAACCCCAGAGATTCAAGA | 811 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTACCAAAGAAACC | |
| | AAAAAATCTTGGGGTACGAACTACTACATCAAGGTTCGTGCGGGTGACAACAA | |
| | GTATATGCACCTGAAAGTGTTTAAGAGCCTGCAGAAAATCCATGCAATCTTCT | |
| | ACGTTGAAGATTTGGTGCTGACGGGCTACCAGGTTGACAAGAACAAAGATGAC | |
| | GAGCTGACGGGTTTC | |
| 45 | ATGATCCCAGGTGGCCTGAGCGAAGCAAAACCAGCAACCCCAGAGATTCAAGA | 812 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTGAATACCCACAT | |
| | TTCCAGGGTTACGGTACGAACTACTACATCAAGGTTCGTGCGGGTGACAACAA | |
| | GTATATGCACCTGAAAGTGTTTAAGAGCCTGATCCTGAACGCAGGTGGTATCA | |
| | GATGGGAAGATTTGGTGCTGACGGGCTACCAGGTTGACAAGAACAAAGATGAC | |
| | GAGCTGACGGGTTTC | |
| 46 | ATGATCCCAGGTGGCCTGAGCGAAGCAAAACCAGCAACCCCAGAGATTCAAGA | 813 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTCAGCATTCTCTG | |
| | TGGTACTTCCAGGGTACGAACTACTACATCAAGGTTCGTGCGGGTGACAACAA | |
| | GTATATGCACCTGAAAGTGTTTAAGAGCCTGTTCAAACATCCAGAAGAAAACC | |
| | CAGTTGAAGATTTGGTGCTGACGGGCTACCAGGTTGACAAGAACAAAGATGAC | |
| | GAGCTGACGGGTTTC | |
| 47 | ATGATCCCAGGTGGCCTGAGCGAAGCAAAACCAGCAACCCCAGAGATTCAAGA | 814 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGGTGTTAAACAGACC | |
| | TGGAACTGGAACGGTACGAACTACTACATCAAGGTTCGTGCGGGTGACAACAA | |
| | GTATATGCACCTGAAAGTGTTTAAGAGCCTGTTCGGTCTGAACGATGATGAAC | |
| | CAGTTGAAGATTTGGTGCTGACGGGCTACCAGGTTGACAAGAACAAAGATGAC | |
| | GAGCTGACGGGTTTC | |
| 48 | ATGATCCCAGGTGGCCTGAGCGAAGCAAAACCAGCAACCCCAGAGATTCAAGA | 815 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCACCCGTAAACATTTC | |
| | CCACAGTACTGGGGTACGAACTACTACATCAAGGTTCGTGCGGGTGACAACAA | |
| | GTATATGCACCTGAAAGTGTTTAAGAGCCTGAACGAACCACTGCCAGTTACCA | |
| | ACATCGAAGATTTGGTGCTGACGGGCTACCAGGTTGACAAGAACAAAGATGAC | |
| | GAGCTGACGGGTTTC | |
| 49 | ATGATCCCAGGTGGCCTGAGCGAAGCAAAACCAGCAACCCCAGAGATTCAAGA | 816 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAAATACTACGCAATC | |
| | TTCGATTACAAAGGTACGAACTACTACATCAAGGTTCGTGCGGGTGACAACAA | |
| | GTATATGCACCTGAAAGTGTTTAAGAGCCTGTTCAAAGAAAAAGTTAGAAACT | |
| | CTATCGAAGATTTGGTGCTGACGGGCTACCAGGTTGACAAGAACAAAGATGAC | |
| | GAGCTGACGGGTTTC | |
| 50 | ATGATCCCAGGTGGCCTGAGCGAAGCAAAACCAGCAACCCCAGAGATTCAAGA | 817 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGGTCGTCAGGTTAAA | |
| | TGGGCATGGACCGGTACGAACTACTACATCAAGGTTCGTGCGGGTGACAACAA | |
| | GTATATGCACCTGAAAGTGTTTAAGAGCCTGTTCAAACAGCATCATAACATCT | |
| | CTGAAGAAGATTTGGTGCTGACGGGCTACCAGGTTGACAAGAACAAAGATGAC | |
| | GAGCTGACGGGTTTC | |
| 51 | ATGATCCCAGGTGGCCTGAGCGAAGCAAAACCAGCAACCCCAGAGATTCAAGA | 818 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTCTCATCATGCATCT | |
| | CCATCTCATATCGGTACGAACTACTACATCAAGGTTCGTGCGGGTGACAACAA | |
| | GTATATGCACCTGAAAGTGTTTAAGAGCCTGACCACCGATCCAAACTGGGATG | |
| | TTCATGAAGATTTGGTGCTGACGGGCTACCAGGTTGACAAGAACAAAGATGAC | |
| | GAGCTGACGGGTTTC | |
| 52 | ATGATCCCAGGTGGCCTGAGCGAAGCAAAACCAGCAACCCCAGAGATTCAAGA | 819 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGGTATCCGTGAAGAT | |
| | TGGAACTGGTCTGGTACGAACTACTACATCAAGGTTCGTGCGGGTGACAACAA | |
| | GTATATGCACCTGAAAGTGTTTAAGAGCCTGTTCGAACCAAAAAAACTGGAAC | |
| | CAGCAGAAGATTTGGTGCTGACGGGCTACCAGGTTGACAAGAACAAAGATGAC | |
| | GAGCTGACGGGTTTC | |
| 53 | ATGATCCCAGGTGGCCTGAGCGAAGCAAAACCAGCAACCCCAGAGATTCAAGA | 820 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGCATGGGATTGGCTG | |
| | CCAGCATACCGTGGTACGAACTACTACATCAAGGTTCGTGCGGGTGACAACAA | |
| | GTATATGCACCTGAAAGTGTTTAAGAGCCTGTTCAAATTCCAGAACGAATCTT | |
| | TCGCAGAAGATTTGGTGCTGACGGGCTACCAGGTTGACAAGAACAAAGATGAC | |
| | GAGCTGACGGGTTTC | |
| 54 | ATGATCCCAGGTGGCCTGAGCGAAGCAAAACCAGCAACCCCAGAGATTCAAGA | 821 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCAACCAGCAGCAG | |
| | ATCAAACATTCTGGTACGAACTACTACATCAAGGTTCGTGCGGGTGACAACAA | |
| | GTATATGCACCTGAAAGTGTTTAAGAGCCTGTGGCAGCATATCCCACCAGATT | |
| | TCTGGGAAGATTTGGTGCTGACGGGCTACCAGGTTGACAAGAACAAAGATGAC | |
| | GAGCTGACGGGTTTC | |
| 55 | ATGATCCCAGGTGGCCTGAGCGAAGCAAAACCAGCAACCCCAGAGATTCAAGA | 822 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGCAGGTTGGTCTCTG | |
| | CCACCACATCTGGGTACGAACTACTACATCAAGGTTCGTGCGGGTGACAACAA | |
| | GTATATGCACCTGAAAGTGTTTAAGAGCCTGATCAACCATGCAGGTCAGAAAT | |
| | CTGATGAAGATTTGGTGCTGACGGGCTACCAGGTTGACAAGAACAAAGATGAC | |
| | GAGCTGACGGGTTTC | |
| 56 | ATGATCCCAGGTGGCCTGAGCGAAGCAAAACCAGCAACCCCAGAGATTCAAGA | 823 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGGTGGTCGTCATCGT | |
| | TGGAACTGGTCTGGTACGAACTACTACATCAAGGTTCGTGCGGGTGACAACAA | |
| | GTATATGCACCTGAAAGTGTTTAAGAGCCTGTTCGAACCATCTGGTCTGGCAG | |
| | CAGAAGAAGATTTGGTGCTGACGGGCTACCAGGTTGACAAGAACAAAGATGAC | |
| | GAGCTGACGGGTTTC | |
| 57 | ATGATCCCAGGTGGCCTGAGCGAAGCAAAACCAGCAACCCCAGAGATTCAAGA | 824 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGAACTGGATGGTGAT | |
| | CATACCTGGCTGGGTACGAACTACTACATCAAGGTTCGTGCGGGTGACAACAA | |
| | GTATATGCACCTGAAAGTGTTTAAGAGCCTGAGACAGCTGCATAGAATCTGGT | |
| | ACTTCGAAGATTTGGTGCTGACGGGCTACCAGGTTGACAAGAACAAAGATGAC | |
| | GAGCTGACGGGTTTC | |
| 58 | ATGATCCCAGGTGGCCTGAGCGAAGCAAAACCAGCAACCCCAGAGATTCAAGA | 825 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAAACAGCTGATCATC | |
| | TGGCATTGGACCGGTACGAACTACTACATCAAGGTTCGTGCGGGTGACAACAA | |
| | GTATATGCACCTGAAAGTGTTTAAGAGCCTGTTCAAAAACTCTAACTCTCATC | |
| | TGGAAGAAGATTTGGTGCTGACGGGCTACCAGGTTGACAAGAACAAAGATGAC | |
| | GAGCTGACGGGTTTC | |
| 59 | ATGATCCCAGGTGGCCTGAGCGAAGCAAAACCAGCAACCCCAGAGATTCAAGA | 826 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTGAACCAGCACTG | |
| | TGGCATTTCCGTGGTACGAACTACTACATCAAGGTTCGTGCGGGTGACAACAA | |
| | GTATATGCACCTGAAAGTGTTTAAGAGCCTGACCTTCAAAGTTGATACCTGGA | |
| | AAGTTGAAGATTTGGTGCTGACGGGCTACCAGGTTGACAAGAACAAAGATGAC | |
| | GAGCTGACGGGTTTC | |
| 60 | ATGATCCCAGGTGGCCTGAGCGAAGCAAAACCAGCAACCCCAGAGATTCAAGA | 827 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCCACAGGTTCAGGAA | |
| | CCATGGTGGATCGGTACGAACTACTACATCAAGGTTCGTGCGGGTGACAACAA | |
| | GTATATGCACCTGAAAGTGTTTAAGAGCCTGTACAAAGCATACATCCAGGGTT | |
| | GGAACGAAGATTTGGTGCTGACGGGCTACCAGGTTGACAAGAACAAAGATGAC | |
| | GAGCTGACGGGTTTC | |
| 61 | ATGATCCCAGGTGGCCTGAGCGAAGCAAAACCAGCAACCCCAGAGATTCAAGA | 828 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCCAACCGGTACCCAT | |
| | CAGTGGGCAGCAGGTACGAACTACTACATCAAGGTTCGTGCGGGTGACAACAA | |
| | GTATATGCACCTGAAAGTGTTTAAGAGCCTGTGGGCATCTCTGGAATCTGGTC | |
| | AGAACGAAGATTTGGTGCTGACGGGCTACCAGGTTGACAAGAACAAAGATGAC | |
| | GAGCTGACGGGTTTC | |
| 62 | ATGATCCCAGGTGGCCTGAGCGAAGCAAAACCAGCAACCCCAGAGATTCAAGA | 829 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTTCCGTCAGCATTTC | |
| | CCAAACTTCTCTGGTACGAACTACTACATCAAGGTTCGTGCGGGTGACAACAA | |
| | GTATATGCACCTGAAAGTGTTTAAGAGCCTGAACGGTCTGGAAGATCCAACCC | |
| | AGGATGAAGATTTGGTGCTGACGGGCTACCAGGTTGACAAGAACAAAGATGAC | |
| | GAGCTGACGGGTTTC | |
| 63 | ATGATCCCAGGTGGCCTGAGCGAAGCAAAACCAGCAACCCCAGAGATTCAAGA | 830 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCAGGAATACGAAGAT | |
| | CAGGAATGGGCAGGTACGAACTACTACATCAAGGTTCGTGCGGGTGACAACAA | |
| | GTATATGCACCTGAAAGTGTTTAAGAGCCTGCATCTGTGGAACCAGCAGATCT | |
| | ACAGAGAAGATTTGGTGCTGACGGGCTACCAGGTTGACAAGAACAAAGATGAC | |
| | GAGCTGACGGGTTTC | |
| 64 | ATGATCCCAGGTGGCCTGAGCGAAGCAAAACCAGCAACCCCAGAGATTCAAGA | 831 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCGCACATATCCAT | |
| | TGGAAATACTTCGGTACGAACTACTACATCAAGGTTCGTGCGGGTGACAACAA | |
| | GTATATGCACCTGAAAGTGTTTAAGAGCCTGCATCTGGCAAAAGGTCAGTGGA | |
| | GAGAAGAAGATTTGGTGCTGACGGGCTACCAGGTTGACAAGAACAAAGATGAC | |
| | GAGCTGACGGGTTTC | |
| 65 | ATGATCCCAGGTGGCCTGAGCGAAGCAAAACCAGCAACCCCAGAGATTCAAGA | 832 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATTACCGTGCAATC | |
| | TGGCATTGGTGGGGTACGAACTACTACATCAAGGTTCGTGCGGGTGACAACAA | |
| | GTATATGCACCTGAAAGTGTTTAAGAGCCTGTTCCATGCAGAAGGTGATATCT | |
| | GGGATGAAGATTTGGTGCTGACGGGCTACCAGGTTGACAAGAACAAAGATGAC | |
| | GAGCTGACGGGTTTC | |
| 66 | ATGATCCCAGGTGGCCTGAGCGAAGCAAAACCAGCAACCCCAGAGATTCAAGA | 833 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAACCTGAAACCAGAT | |
| | GTTCAGGGTTCTGGTACGAACTACTACATCAAGGTTCGTGCGGGTGACAACAA | |
| | GTATATGCACCTGAAAGTGTTTAAGAGCCTGATCGTTGTTAAAAGATACTGGT | |
| | ACGTTGAAGATTTGGTGCTGACGGGCTACCAGGTTGACAAGAACAAAGATGAC | |
| | GAGCTGACGGGTTTC | |
| 67 | ATGATCCCAGGTGGCCTGAGCGAAGCAAAACCAGCAACCCCAGAGATTCAAGA | 834 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTCTACCAAAAACCTG | |
| | TGGCGTTGGTCTGGTACGAACTACTACATCAAGGTTCGTGCGGGTGACAACAA | |
| | GTATATGCACCTGAAAGTGTTTAAGAGCCTGTTCAACGGTGCAGGTGGTCCAG | |
| | ATTCTGAAGATTTGGTGCTGACGGGCTACCAGGTTGACAAGAACAAAGATGAC | |
| | GAGCTGACGGGTTTC | |
| 68 | ATGATCCCAGGTGGCCTGAGCGAAGCAAAACCAGCAACCCCAGAGATTCAAGA | 835 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTGTTCAGGGTCTG | |
| | TGGAACTTCCAGGGTACGAACTACTACATCAAGGTTCGTGCGGGTGACAACAA | |
| | GTATATGCACCTGAAAGTGTTTAAGAGCCTGGGACATCATGGTGATCATGAAA | |
| | AATGGGAAGATTTGGTGCTGACGGGCTACCAGGTTGACAAGAACAAAGATGAC | |
| | GAGCTGACGGGTTTC | |
| 69 | ATGATCCCAGGTGGCCTGAGCGAAGCAAAACCAGCAACCCCAGAGATTCAAGA | 836 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACAAAGTTGATAAC | |
| | AACAACTACCAGGGTACGAACTACTACATCAAGGTTCGTGCGGGTGACAACAA | |
| | GTATATGCACCTGAAAGTGTTTAAGAGCCTGATCACCTGGCAGCTGAACCAGC | |
| | CAAGAGAAGATTTGGTGCTGACGGGCTACCAGGTTGACAAGAACAAAGATGAC | |
| | GAGCTGACGGGTTTC | |
| 70 | ATGATCCCAGGTGGCCTGAGCGAAGCAAAACCAGCAACCCCAGAGATTCAAGA | 837 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCGAAGAACCATCT | |
| | GCATTCTGGTGGGGTACGAACTACTACATCAAGGTTCGTGCGGGTGACAACAA | |
| | GTATATGCACCTGAAAGTGTTTAAGAGCCTGCTGAGAGTTAGACATTCTGCAT | |
| | ACAGAGAAGATTTGGTGCTGACGGGCTACCAGGTTGACAAGAACAAAGATGAC | |
| | GAGCTGACGGGTTTC | |
| 71 | ATGATCCCAGGTGGCCTGAGCGAAGCAAAACCAGCAACCCCAGAGATTCAAGA | 838 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGCATCTTTCCCAGAA | |
| | ATCTCTTGGTACGGTACGAACTACTACATCAAGGTTCGTGCGGGTGACAACAA | |
| | GTATATGCACCTGAAAGTGTTTAAGAGCCTGAAAAACTACCCAAAAACCGGTG | |
| | TTACCGAAGATTTGGTGCTGACGGGCTACCAGGTTGACAAGAACAAAGATGAC | |
| | GAGCTGACGGGTTTC | |
| 72 | ATGATCCCAGGTGGCCTGAGCGAAGCAAAACCAGCAACCCCAGAGATTCAAGA | 839 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCCATGAACCAGAA | |
| | ATCTGGTGGTACGGTACGAACTACTACATCAAGGTTCGTGCGGGTGACAACAA | |
| | GTATATGCACCTGAAAGTGTTTAAGAGCCTGCATAAAGCAGATAGACAGAAAG | |
| | AAAGAGAAGATTTGGTGCTGACGGGCTACCAGGTTGACAAGAACAAAGATGAC | |
| | GAGCTGACGGGTTTC | |
| 73 | ATGATCCCAGGTGGCCTGAGCGAAGCAAAACCAGCAACCCCAGAGATTCAAGA | 840 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATTGGGGTTGGGGT | |
| | ATCCCATGGGTTGGTACGAACTACTACATCAAGGTTCGTGCGGGTGACAACAA | |
| | GTATATGCACCTGAAAGTGTTTAAGAGCCTGTGGCTGTACGGTGTTAAAAGAC | |
| | TGCCAGAAGATTTGGTGCTGACGGGCTACCAGGTTGACAAGAACAAAGATGAC | |
| | GAGCTGACGGGTTTC | |
| 74 | ATGATCCCAGGTGGCCTGAGCGAAGCAAAACCAGCAACCCCAGAGATTCAAGA | 841 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTCGTTGGGAACTG | |
| | TGGTCTTTCCAGGGTACGAACTACTACATCAAGGTTCGTGCGGGTGACAACAA | |
| | GTATATGCACCTGAAAGTGTTTAAGAGCCTGTGGCAGGAACATACCGATTCTG | |
| | TTGAAGAAGATTTGGTGCTGACGGGCTACCAGGTTGACAAGAACAAAGATGAC | |
| | GAGCTGACGGGTTTC | |
| 75 | ATGATCCCAGGTGGCCTGAGCGAAGCAAAACCAGCAACCCCAGAGATTCAAGA | 842 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCAACCAGCATGAA | |
| | AACTTCAAACAGGGTACGAACTACTACATCAAGGTTCGTGCGGGTGACAACAA | |
| | GTATATGCACCTGAAAGTGTTTAAGAGCCTGCATCTGTTCCATGGTCTGTGGT | |
| | ACATCGAAGATTTGGTGCTGACGGGCTACCAGGTTGACAAGAACAAAGATGAC | |
| | GAGCTGACGGGTTTC | |
| 76 | ATGATCCCAGGTGGCCTGAGCGAAGCAAAACCAGCAACCCCAGAGATTCAAGA | 843 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCATGATTTCACCATC | |
| | CGTTACCCATGGGGTACGAACTACTACATCAAGGTTCGTGCGGGTGACAACAA | |
| | GTATATGCACCTGAAAGTGTTTAAGAGCCTGGAATACCATGTTACCAAAAGAG | |
| | ATCAGGAAGATTTGGTGCTGACGGGCTACCAGGTTGACAAGAACAAAGATGAC | |
| | GAGCTGACGGGTTTC | |
| 77 | ATGATCCCAGGTGGCCTGAGCGAAGCAAAACCAGCAACCCCAGAGATTCAAGA | 844 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTGGGATCGTCCACGT | |
| | TGGGGTTGGACCGGTACGAACTACTACATCAAGGTTCGTGCGGGTGACAACAA | |
| | GTATATGCACCTGAAAGTGTTTAAGAGCCTGTTCAAAGAATCTTCTAGAAACG | |
| | ATGAAGAAGATTTGGTGCTGACGGGCTACCAGGTTGACAAGAACAAAGATGAC | |
| | GAGCTGACGGGTTTC | |
| 78 | ATGATCCCAGGTGGCCTGAGCGAAGCAAAACCAGCAACCCCAGAGATTCAAGA | 845 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATCAGGGTTGGTGG | |
| | GAAAACTGGGCAGGTACGAACTACTACATCAAGGTTCGTGCGGGTGACAACAA | |
| | GTATATGCACCTGAAAGTGTTTAAGAGCCTGATCTGGGAAAACAACCTGAGAA | |
| | TCCATGAAGATTTGGTGCTGACGGGCTACCAGGTTGACAAGAACAAAGATGAC | |
| | GAGCTGACGGGTTTC | |
| 79 | ATGATCCCAGGTGGCCTGAGCGAAGCAAAACCAGCAACCCCAGAGATTCAAGA | 846 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCTGGCATTCTGAA | |
| | TGGTCTTACACCGGTACGAACTACTACATCAAGGTTCGTGCGGGTGACAACAA | |
| | GTATATGCACCTGAAAGTGTTTAAGAGCCTGTTCACCAGACATTCTGAACCAT | |
| | GGGAAGAAGATTTGGTGCTGACGGGCTACCAGGTTGACAAGAACAAAGATGAC | |
| | GAGCTGACGGGTTTC | |
| 80 | ATGATCCCAGGTGGCCTGAGCGAAGCAAAACCAGCAACCCCAGAGATTCAAGA | 847 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTCGTAAACATTTC | |
| | CCACAGTGGCCAGGTACGAACTACTACATCAAGGTTCGTGCGGGTGACAACAA | |
| | GTATATGCACCTGAAAGTGTTTAAGAGCCTGGATCTGCAGCCAAGAGAAGTTT | |
| | TCCAGGAAGATTTGGTGCTGACGGGCTACCAGGTTGACAAGAACAAAGATGAC | |
| | GAGCTGACGGGTTTC | |
| 81 | ATGATCCCAGGTGGCCTGAGCGAAGCAAAACCAGCAACCCCAGAGATTCAAGA | 848 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAAAGCATACGTTATC | |
| | TGGTCTTACAAAGGTACGAACTACTACATCAAGGTTCGTGCGGGTGACAACAA | |
| | GTATATGCACCTGAAAGTGTTTAAGAGCCTGTTCTACGGTGAACCAAGAGGTA | |
| | AAGGTGAAGATTTGGTGCTGACGGGCTACCAGGTTGACAAGAACAAAGATGAC | |
| | GAGCTGACGGGTTTC | |
| 82 | ATGATCCCAGGTGGCCTGAGCGAAGCAAAACCAGCAACCCCAGAGATTCAAGA | 849 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTTCTGGCAGCTGCAT | |
| | CGTTACGGTTTCGGTACGAACTACTACATCAAGGTTCGTGCGGGTGACAACAA | |
| | GTATATGCACCTGAAAGTGTTTAAGAGCCTGGAATGGAACAGACTGTCTCCAC | |
| | TGTGGGAAGATTTGGTGCTGACGGGCTACCAGGTTGACAAGAACAAAGATGAC | |
| | GAGCTGACGGGTTTC | |
| 83 | ATGATCCCAGGTGGCCTGAGCGAAGCAAAACCAGCAACCCCAGAGATTCAAGA | 850 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCTACCATGAACTG | |
| | TGGAACTTCTACGGTACGAACTACTACATCAAGGTTCGTGCGGGTGACAACAA | |
| | GTATATGCACCTGAAAGTGTTTAAGAGCCTGGATAACAACGGTGATGGTAACT | |
| | GGCAGGAAGATTTGGTGCTGACGGGCTACCAGGTTGACAAGAACAAAGATGAC | |
| | GAGCTGACGGGTTTC | |
| 84 | ATGATCCCAGGTGGCCTGAGCGAAGCAAAACCAGCAACCCCAGAGATTCAAGA | 851 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGATCCGTCATCTG | |
| | TGGTCTTGGTCTGGTACGAACTACTACATCAAGGTTCGTGCGGGTGACAACAA | |
| | GTATATGCACCTGAAAGTGTTTAAGAGCCTGTTCGAAGGTGAAAACCATCAGA | |
| | TCGATGAAGATTTGGTGCTGACGGGCTACCAGGTTGACAAGAACAAAGATGAC | |
| | GAGCTGACGGGTTTC | |
| 85 | ATGATCCCAGGTGGCCTGAGCGAAGCAAAACCAGCAACCCCAGAGATTCAAGA | 852 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAACGATGGTAAATCT | |
| | CTGGTTCTGGCAGGTACGAACTACTACATCAAGGTTCGTGCGGGTGACAACAA | |
| | GTATATGCACCTGAAAGTGTTTAAGAGCCTGAGACTGTGGGATGATAGAGTTT | |
| | CTGGTGAAGATTTGGTGCTGACGGGCTACCAGGTTGACAAGAACAAAGATGAC | |
| | GAGCTGACGGGTTTC | |
| 86 | ATGATCCCAGGTGGCCTGAGCGAAGCAAAACCAGCAACCCCAGAGATTCAAGA | 853 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATGTTCGTGGTCAG | |
| | TGGGCATGGACCGGTACGAACTACTACATCAAGGTTCGTGCGGGTGACAACAA | |
| | GTATATGCACCTGAAAGTGTTTAAGAGCCTGTTCCAGCCAAGAACCAAAGCAA | |
| | CCGAAGAAGATTTGGTGCTGACGGGCTACCAGGTTGACAAGAACAAAGATGAC | |
| | GAGCTGACGGGTTTC | |
| 87 | ATGATCCCAGGTGGCCTGAGCGAAGCAAAACCAGCAACCCCAGAGATTCAAGA | 854 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAAACGTCTGCATCTG | |
| | CCACAGTACCCAGGTACGAACTACTACATCAAGGTTCGTGCGGGTGACAACAA | |
| | GTATATGCACCTGAAAGTGTTTAAGAGCCTGGGTAACATCGAACCAATCACCG | |
| | ATCTGGAAGATTTGGTGCTGACGGGCTACCAGGTTGACAAGAACAAAGATGAC | |
| | GAGCTGACGGGTTTC | |
| 88 | ATGATCCCAGGTGGCCTGAGCGAAGCAAAACCAGCAACCCCAGAGATTCAAGA | 855 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGCAGTTCATGCACCA | |
| | CAGTACGCACCAGGTACGAACTACTACATCAAGGTTCGTGCGGGTGACAACAA | |
| | GTATATGCACCTGAAAGTGTTTAAGAGCCTGTGGTCTGAAAAAGTTGCAGTTT | |
| | TCCCAGAAGATTTGGTGCTGACGGGCTACCAGGTTGACAAGAACAAAGATGAC | |
| | GAGCTGACGGGTTTC | |
| 89 | ATGATCCCAGGTGGCCTGAGCGAAGCAAAACCAGCAACCCCAGAGATTCAAGA | 856 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATTTCCGTGTTGCA | |
| | TGGCATTGGGTTGGTACGAACTACTACATCAAGGTTCGTGCGGGTGACAACAA | |
| | GTATATGCACCTGAAAGTGTTTAAGAGCCTGTTCAACCATAGACCAGGTAAAC | |
| | CAGATGAAGATTTGGTGCTGACGGGCTACCAGGTTGACAAGAACAAAGATGAC | |
| | GAGCTGACGGGTTTC | |
| 90 | ATGATCCCAGGTGGCCTGAGCGAAGCAAAACCAGCAACCCCAGAGATTCAAGA | 857 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGGGTTGGTGGTTC | |
| | GGTATCAACCAGGGTACGAACTACTACATCAAGGTTCGTGCGGGTGACAACAA | |
| | GTATATGCACCTGAAAGTGTTTAAGAGCCTGGCACATCTGCAGTACGAATGGG | |
| | GTGGTGAAGATTTGGTGCTGACGGGCTACCAGGTTGACAAGAACAAAGATGAC | |
| | GAGCTGACGGGTTTC | |
| 91 | ATGATCCCAGGTGGCCTGAGCGAAGCAAAACCAGCAACCCCAGAGATTCAAGA | 858 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAACCAGGAACATTTC | |
| | CGTCGTTTCATCGGTACGAACTACTACATCAAGGTTCGTGCGGGTGACAACAA | |
| | GTATATGCACCTGAAAGTGTTTAAGAGCCTGAACCCATGGCTGCATCCACAGG | |
| | ATTTCGAAGATTTGGTGCTGACGGGCTACCAGGTTGACAAGAACAAAGATGAC | |
| | GAGCTGACGGGTTTC | |
| 92 | ATGATCCCAGGTGGCCTGAGCGAAGCAAAACCAGCAACCCCAGAGATTCAAGA | 859 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACATCGATGATGATCTG | |
| | TGGTCTGGTACGAACTACTACATCAAGGTTCGTGCGGGTGACAACAAGTATAT | |
| | GCACCTGAAAGTGTTTAAGAGCCTGATCCTGTGGCCAACCAAACCAGAAAGAG | |
| | AAGATTTGGTGCTGACGGGCTACCAGGTTGACAAGAACAAAGATGACGAGCTG | |
| | ACGGGTTTC | |
| 93 | ATGATCCCAGGTGGCCTGAGCGAAGCAAAACCAGCAACCCCAGAGATTCAAGA | 860 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACCAGTGGGGTGTTGAT | |
| | CTGGTTTACACCGGTACGAACTACTACATCAAGGTTCGTGCGGGTGACAACAA | |
| | GTATATGCACCTGAAAGTGTTTAAGAGCCTGATCGTTAAAAGAGCAGTTATCC | |
| | TGTGGGAAGATTTGGTGCTGACGGGCTACCAGGTTGACAAGAACAAAGATGAC | |
| | GAGCTGACGGGTTTC | |
| 94 | ATGATCCCAGGTGGCCTGAGCGAAGCAAAACCAGCAACCCCAGAGATTCAAGA | 861 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACAAAGTTTACCTGGTT | |
| | TGGGCATGGACCGGTACGAACTACTACATCAAGGTTCGTGCGGGTGACAACAA | |
| | GTATATGCACCTGAAAGTGTTTAAGAGCCTGTTCCAGAAAGGTTCTAACAAAC | |
| | CAGAAGAAGATTTGGTGCTGACGGGCTACCAGGTTGACAAGAACAAAGATGAC | |
| | GAGCTGACGGGTTTC | |
| 95 | ATGATCCCAGGTGGCCTGAGCGAAGCAAAACCAGCAACCCCAGAGATTCAAGA | 862 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACACCATCCATCCACAG | |
| | GCAAAATACTGGGGTACGAACTACTACATCAAGGTTCGTGCGGGTGACAACAA | |
| | GTATATGCACCTGAAAGTGTTTAAGAGCCTGTGGAAAAACCTGCTGAAACCAA | |
| | TCCATGAAGATTTGGTGCTGACGGGCTACCAGGTTGACAAGAACAAAGATGAC | |
| | GAGCTGACGGGTTTC | |
| 96 | ATGATCCCAGGTGGCCTGAGCGAAGCAAAACCAGCAACCCCAGAGATTCAAGA | 863 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACTTCAACCAGTCTCGT | |
| | CATGGTCATGGTACGAACTACTACATCAAGGTTCGTGCGGGTGACAACAAGTA | |
| | TATGCACCTGAAAGTGTTTAAGAGCCTGGAACTGCTGCATGGTCTGTGGTGGG | |
| | TTGAAGATTTGGTGCTGACGGGCTACCAGGTTGACAAGAACAAAGATGACGAG | |
| | CTGACGGGTTTC | |
| 97 | ATGATCCCAGGTGGCCTGAGCGAAGCAAAACCAGCAACCCCAGAGATTCAAGA | 864 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACGAACTGGATGATGCA | |
| | GTTAAATACTGGGGTACGAACTACTACATCAAGGTTCGTGCGGGTGACAACAA | |
| | GTATATGCACCTGAAAGTGTTTAAGAGCCTGTGGTGGAAAACCATCTCTGCAG | |
| | ATTTCGAAGATTTGGTGCTGACGGGCTACCAGGTTGACAAGAACAAAGATGAC | |
| | GAGCTGACGGGTTTC | |
| 98 | ATGATCCCAGGTGGCCTGAGCGAAGCAAAACCAGCAACCCCAGAGATTCAAGA | 865 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACTGGGCAATCTGGCGT | |
| | GATAAAAAAGTTGGTACGAACTACTACATCAAGGTTCGTGCGGGTGACAACAA | |
| | GTATATGCACCTGAAAGTGTTTAAGAGCCTGCCAATCTTCGAAATCGTTAGAG | |
| | AAGGTGAAGATTTGGTGCTGACGGGCTACCAGGTTGACAAGAACAAAGATGAC | |
| | GAGCTGACGGGTTTC | |
| 99 | ATGATCCCAGGTGGCCTGAGCGAAGCAAAACCAGCAACCCCAGAGATTCAAGA | 866 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACACCTGGGGTGAACGT | |
| | CTGCATTACCCAGGTACGAACTACTACATCAAGGTTCGTGCGGGTGACAACAA | |
| | GTATATGCACCTGAAAGTGTTTAAGAGCCTGTGGCATAAACCAAAAGATAACA | |
| | TCATCGAAGATTTGGTGCTGACGGGCTACCAGGTTGACAAGAACAAAGATGAC | |
| | GAGCTGACGGGTTTC | |
| 100 | ATGATCCCAGGTGGCCTGAGCGAAGCAAAACCAGCAACCCCAGAGATTCAAGA | 867 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACCATACCGATGTTGAT | |
| | TGGCATTTCTGGGGTACGAACTACTACATCAAGGTTCGTGCGGGTGACAACAA | |
| | GTATATGCACCTGAAAGTGTTTAAGAGCCTGGCACCAGCAGCACCACCATGGG | |
| | AATTCGAAGATTTGGTGCTGACGGGCTACCAGGTTGACAAGAACAAAGATGAC | |
| | GAGCTGACGGGTTTC | |
| 101 | ATGATCCCAGGTGGCCTGAGCGAAGCAAAACCAGCAACCCCAGAGATTCAAGA | 868 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACCATCATCATCGTTTC | |
| | GCAAAATGGTGGGGTACGAACTACTACATCAAGGTTCGTGCGGGTGACAACAA | |
| | GTATATGCACCTGAAAGTGTTTAAGAGCCTGTGGTTCAACGAAAACAGAGAAG | |
| | ATACCGAAGATTTGGTGCTGACGGGCTACCAGGTTGACAAGAACAAAGATGAC | |
| | GAGCTGACGGGTTTC | |
| 102 | ATGATCCCAGGTGGCCTGAGCGAAGCAAAACCAGCAACCCCAGAGATTCAAGA | 869 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACCGTAAAGCAGCAGTT | |
| | GTTGCACGTCATGGTACGAACTACTACATCAAGGTTCGTGCGGGTGACAACAA | |
| | GTATATGCACCTGAAAGTGTTTAAGAGCCTGTGGCCATTCCTGGTTATCGAAA | |
| | AATACGAAGATTTGGTGCTGACGGGCTACCAGGTTGACAAGAACAAAGATGAC | |
| | GAGCTGACGGGTTTC | |
| 103 | ATGATCCCAGGTGGCCTGAGCGAAGCAAAACCAGCAACCCCAGAGATTCAAGA | 870 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACTTCTCTTTCCCACTG | |
| | GTTCATTGGTACGGTACGAACTACTACATCAAGGTTCGTGCGGGTGACAACAA | |
| | GTATATGCACCTGAAAGTGTTTAAGAGCCTGAGATTCAGAGATGGTGGTCATC | |
| | ATGTTGAAGATTTGGTGCTGACGGGCTACCAGGTTGACAAGAACAAAGATGAC | |
| | GAGCTGACGGGTTTC | |
| 104 | ATGATCCCAGGTGGCCTGAGCGAAGCAAAACCAGCAACCCCAGAGATTCAAGA | 871 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACAACGCACATCCACTG | |
| | GTTAAAAACTGGGGTACGAACTACTACATCAAGGTTCGTGCGGGTGACAACAA | |
| | GTATATGCACCTGAAAGTGTTTAAGAGCCTGTGGAGAAACGATACCTCTTCTA | |
| | TCTACGAAGATTTGGTGCTGACGGGCTACCAGGTTGACAAGAACAAAGATGAC | |
| | GAGCTGACGGGTTTC | |
| 105 | ATGATCCCAGGTGGCCTGAGCGAAGCAAAACCAGCAACCCCAGAGATTCAAGA | 872 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACGAAACCTCTGAAAAC | |
| | GTTATCCAGTGGGGTACGAACTACTACATCAAGGTTCGTGCGGGTGACAACAA | |
| | GTATATGCACCTGAAAGTGTTTAAGAGCCTGTGGCCAGCAGATATCCTGACCC | |
| | AGCATGAAGATTTGGTGCTGACGGGCTACCAGGTTGACAAGAACAAAGATGAC | |
| | GAGCTGACGGGTTTC | |
| 106 | ATGATCCCAGGTGGCCTGAGCGAAGCAAAACCAGCAACCCCAGAGATTCAAGA | 873 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACTTCTCTGATCTGGAA | |
| | TGGGAAGGTCAGGGTACGAACTACTACATCAAGGTTCGTGCGGGTGACAACAA | |
| | GTATATGCACCTGAAAGTGTTTAAGAGCCTGGAAAAAAAGTGGGGTTGGATCT | |
| | ACATCGAAGATTTGGTGCTGACGGGCTACCAGGTTGACAAGAACAAAGATGAC | |
| | GAGCTGACGGGTTTC | |
| 107 | ATGATCCCAGGTGGCCTGAGCGAAGCAAAACCAGCAACCCCAGAGATTCAAGA | 874 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACTTCACCGATATCCAT | |
| | TGGTCTACCTGGGGTACGAACTACTACATCAAGGTTCGTGCGGGTGACAACAA | |
| | GTATATGCACCTGAAAGTGTTTAAGAGCCTGATCCCAGCATGGCAGCAGATCC | |
| | ATGTTGAAGATTTGGTGCTGACGGGCTACCAGGTTGACAAGAACAAAGATGAC | |
| | GAGCTGACGGGTTTC | |
| 108 | ATGATCCCAGGTGGCCTGAGCGAAGCAAAACCAGCAACCCCAGAGATTCAAGA | 875 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACCGTGAACATGCATCT | |
| | GTTAAATTCTGGGGTACGAACTACTACATCAAGGTTCGTGCGGGTGACAACAA | |
| | GTATATGCACCTGAAAGTGTTTAAGAGCCTGTGGCTGGAAGCAACCCCACATA | |
| | CCCAGGAAGATTTGGTGCTGACGGGCTACCAGGTTGACAAGAACAAAGATGAC | |
| | GAGCTGACGGGTTTC | |
| 109 | ATGATCCCAGGTGGCCTGAGCGAAGCAAAACCAGCAACCCCAGAGATTCAAGA | 876 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACATCTCTGGTGTTGGT | |
| | CTGGTTCCAAACGGTACGAACTACTACATCAAGGTTCGTGCGGGTGACAACAA | |
| | GTATATGCACCTGAAAGTGTTTAAGAGCCTGAACTGGAACTACCCACAGCAGC | |
| | CATTCGAAGATTTGGTGCTGACGGGCTACCAGGTTGACAAGAACAAAGATGAC | |
| | GAGCTGACGGGTTTC | |
| 110 | ATGATCCCAGGTGGCCTGAGCGAAGCAAAACCAGCAACCCCAGAGATTCAAGA | 877 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACCATACCCAGCCAAAA | |
| | GCACTGGATTGGGGTACGAACTACTACATCAAGGTTCGTGCGGGTGACAACAA | |
| | GTATATGCACCTGAAAGTGTTTAAGAGCCTGTGGGGTTCTATCCTGACCGATG | |
| | GTCCAGAAGATTTGGTGCTGACGGGCTACCAGGTTGACAAGAACAAAGATGAC | |
| | GAGCTGACGGGTTTC | |
| 111 | ATGATCCCAGGTGGCCTGAGCGAAGCAAAACCAGCAACCCCAGAGATTCAAGA | 878 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACATCCAGGATCGTCTG | |
| | GGTGGTTTCATCGGTACGAACTACTACATCAAGGTTCGTGCGGGTGACAACAA | |
| | GTATATGCACCTGAAAGTGTTTAAGAGCCTGCATTTCTACAGAGGTATCCTGT | |
| | ACAGAGAAGATTTGGTGCTGACGGGCTACCAGGTTGACAAGAACAAAGATGAC | |
| | GAGCTGACGGGTTTC | |
| 112 | ATGATCCCAGGTGGCCTGAGCGAAGCAAAACCAGCAACCCCAGAGATTCAAGA | 879 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACCGTTTCTACCATCAG | |
| | TGGCATTTCCTGGGTACGAACTACTACATCAAGGTTCGTGCGGGTGACAACAA | |
| | GTATATGCACCTGAAAGTGTTTAAGAGCCTGGATCTGAGAGCACCAAGAAACG | |
| | ATAACGAAGATTTGGTGCTGACGGGCTACCAGGTTGACAAGAACAAAGATGAC | |
| | GAGCTGACGGGTTTC | |
| 113 | ATGATCCCAGGTGGCCTGAGCGAAGCAAAACCAGCAACCCCAGAGATTCAAGA | 880 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACTGGGTTACCCGTCAT | |
| | CATACCCTGGTTGGTACGAACTACTACATCAAGGTTCGTGCGGGTGACAACAA | |
| | GTATATGCACCTGAAAGTGTTTAAGAGCCTGCCACCATTCAAACATTGGCATC | |
| | TGGAAGATTTGGTGCTGACGGGCTACCAGGTTGACAAGAACAAAGATGACGAG | |
| | CTGACGGGTTTC | |
| 114 | ATGATCCCAGGTGGCCTGAGCGAAGCAAAACCAGCAACCCCAGAGATTCAAGA | 881 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACCAGTCTTCTGATCTG | |
| | GCAAAACTGTGGGGTACGAACTACTACATCAAGGTTCGTGCGGGTGACAACAA | |
| | GTATATGCACCTGAAAGTGTTTAAGAGCCTGTGGAACCTGAACTCTGATAAAT | |
| | CTTCTGAAGATTTGGTGCTGACGGGCTACCAGGTTGACAAGAACAAAGATGAC | |
| | GAGCTGACGGGTTTC | |
| 115 | ATGATCCCAGGTGGCCTGAGCGAAGCAAAACCAGCAACCCCAGAGATTCAAGA | 882 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACCGTTTCTACCATCAG | |
| | TGGCATTTCCTGGGTACGAACTACTACATCAAGGTTCGTGCGGGTGACAACAA | |
| | GTATATGCACCTGAAAGTGTTTAAGAGCCTGGATCTGAGAGCACCAAGAAACG | |
| | ATAACGAAGATTTGGTGCTGACGGGCTACCAGGTTGACAAGAACAAAGATGAC | |
| | GAGCTGACGGGTTTC | |
| 116 | ATGATCCCAGGTGGCCTGAGCGAAGCAAAACCAGCAACCCCAGAGATTCAAGA | 883 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACTGGGTTACCCGTCAT | |
| | CATACCCTGGTTGGTACGAACTACTACATCAAGGTTCGTGCGGGTGACAACAA | |
| | GTATATGCACCTGAAAGTGTTTAAGAGCCTGCCACCATTCAAACATTGGCATC | |
| | TGGAAGATTTGGTGCTGACGGGCTACCAGGTTGACAAGAACAAAGATGACGAG | |
| | CTGACGGGTTTC | |
| 117 | ATGATCCCAGGTGGCCTGAGCGAAGCAAAACCAGCAACCCCAGAGATTCAAGA | 884 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACCATGAAGTTCGTAAA | |
| | ACCTACGAATTCGGTACGAACTACTACATCAAGGTTCGTGCGGGTGACAACAA | |
| | GTATATGCACCTGAAAGTGTTTAAGAGCCTGTGGTTCGGTGGTTGGCCACTGG | |
| | CACAGGAAGATTTGGTGCTGACGGGCTACCAGGTTGACAAGAACAAAGATGAC | |
| | GAGCTGACGGGTTTC | |
| 118 | ATGATCCCAGGTGGCCTGAGCGAAGCAAAACCAGCAACCCCAGAGATTCAAGA | 885 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACGTTATCTCTATCCCA | |
| | CTGGTTTGGAACGGTACGAACTACTACATCAAGGTTCGTGCGGGTGACAACAA | |
| | GTATATGCACCTGAAAGTGTTTAAGAGCCTGATCTTCCAGGAATACTACCCAC | |
| | ATAGAGAAGATTTGGTGCTGACGGGCTACCAGGTTGACAAGAACAAAGATGAC | |
| | GAGCTGACGGGTTTC | |
| 119 | ATGATCCCAGGTGGCCTGAGCGAAGCAAAACCAGCAACCCCAGAGATTCAAGA | 886 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACTCTTGGGTTTTCAAA | |
| | CCATTCTACCACGGTACGAACTACTACATCAAGGTTCGTGCGGGTGACAACAA | |
| | GTATATGCACCTGAAAGTGTTTAAGAGCCTGTCTGGTCCAGAATTCTTCTCTA | |
| | GACATGAAGATTTGGTGCTGACGGGCTACCAGGTTGACAAGAACAAAGATGAC | |
| | GAGCTGACGGGTTTC | |
| 120 | ATGATCCCAGGTGGCCTGAGCGAAGCAAAACCAGCAACCCCAGAGATTCAAGA | 887 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACCCAGAAGTTTCTATC | |
| | ACCCGTTGGCAGGGTACGAACTACTACATCAAGGTTCGTGCGGGTGACAACAA | |
| | GTATATGCACCTGAAAGTGTTTAAGAGCCTGTACGAAACCCATGGTAACATCA | |
| | TCACCGAAGATTTGGTGCTGACGGGCTACCAGGTTGACAAGAACAAAGATGAC | |
| | GAGCTGACGGGTTTC | |
| 121 | ATGATCCCAGGTGGCCTGAGCGAAGCAAAACCAGCAACCCCAGAGATTCAAGA | 888 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACGGTAACTTCGCACAT | |
| | GAACGTTGGCAGGGTACGAACTACTACATCAAGGTTCGTGCGGGTGACAACAA | |
| | GTATATGCACCTGAAAGTGTTTAAGAGCCTGTACATCGCAAGAGATTTCGATT | |
| | GGACCGAAGATTTGGTGCTGACGGGCTACCAGGTTGACAAGAACAAAGATGAC | |
| | GAGCTGACGGGTTTC | |
| 122 | ATGATCCCAGGTGGCCTGAGCGAAGCAAAACCAGCAACCCCAGAGATTCAAGA | 889 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACGTTATCCATATCCCA | |
| | CATCAGTGGCATGGTACGAACTACTACATCAAGGTTCGTGCGGGTGACAACAA | |
| | GTATATGCACCTGAAAGTGTTTAAGAGCCTGATCTTCCATGAACCAAAACCAC | |
| | CAAGAGAAGATTTGGTGCTGACGGGCTACCAGGTTGACAAGAACAAAGATGAC | |
| | GAGCTGACGGGTTTC | |
| 123 | ATGATCCCAGGTGGCCTGAGCGAAGCAAAACCAGCAACCCCAGAGATTCAAGA | 890 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACAACCTGGTTTACTAC | |
| | CAGCGTGATTGGGGTACGAACTACTACATCAAGGTTCGTGCGGGTGACAACAA | |
| | GTATATGCACCTGAAAGTGTTTAAGAGCCTGGAACCAGGTAGATGGAGATGGC | |
| | CAAAAGAAGATTTGGTGCTGACGGGCTACCAGGTTGACAAGAACAAAGATGAC | |
| | GAGCTGACGGGTTTC | |
| 124 | ATGATCCCAGGTGGCCTGAGCGAAGCAAAACCAGCAACCCCAGAGATTCAAGA | 891 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGACCAGGTTCTGTGGGAA | |
| | ATCTTCGAACATGGTACGAACTACTACATCAAGGTTCGTGCGGGTGACAACAA | |
| | GTATATGCACCTGAAAGTGTTTAAGAGCCTGTTCCATCAGTGGTCTCCAGCAC | |
| | TGCATGAAGATTTGGTGCTGACGGGCTACCAGGTTGACAAGAACAAAGATGAC | |
| | GAGCTGACGGGTTTC | |
| 125 | ATGATCCCAGGTGGCCTGAGCGAAGCAAAACCAGCAACCCCAGAGATTCAAGA | 892 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCACCCGTCTGCATTTC | |
| | CCACAGTACCAGGGTACGAACTACTACATCAAGGTTCGTGCGGGTGACAACAA | |
| | GTATATGCACCTGAAAGTGTTTAAGAGCCTGGAAATCGAAGTTAACCCATACG | |
| | CAAACGAAGATTTGGTGCTGACGGGCTACCAGGTTGACAAGAACAAAGATGAC | |
| | GAGCTGACGGGTTTC | |
| 126 | ATGATCCCAGGTGGCCTGAGCGAAGCAAAACCAGCAACCCCAGAGATTCAAGA | 893 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACGAACCACATCAT | |
| | CCATACCCATGGGGTACGAACTACTACATCAAGGTTCGTGCGGGTGACAACAA | |
| | GTATATGCACCTGAAAGTGTTTAAGAGCCTGCAGTACCATCCACCATACGAAA | |
| | GATTCGAAGATTTGGTGCTGACGGGCTACCAGGTTGACAAGAACAAAGATGAC | |
| | GAGCTGACGGGTTTC | |
| 127 | ATGATCCCAGGTGGCCTGAGCGAAGCAAAACCAGCAACCCCAGAGATTCAAGA | 894 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCACCCAGGCAATCCCA | |
| | TGGCGTGAATGGGGTACGAACTACTACATCAAGGTTCGTGCGGGTGACAACAA | |
| | GTATATGCACCTGAAAGTGTTTAAGAGCCTGATCCCAGAATGGCCAAAAGATG | |
| | CATCTGAAGATTTGGTGCTGACGGGCTACCAGGTTGACAAGAACAAAGATGAC | |
| | GAGCTGACGGGTTTC | |
| 128 | ATGATCCCAGGTGGCCTGAGCGAAGCAAAACCAGCAACCCCAGAGATTCAAGA | 895 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCCAGAAGTTGATAAA | |
| | GCAGAATGGTGGGGTACGAACTACTACATCAAGGTTCGTGCGGGTGACAACAA | |
| | GTATATGCACCTGAAAGTGTTTAAGAGCCTGATCAGATACGGTCTGAGACATG | |
| | AAAGAGAAGATTTGGTGCTGACGGGCTACCAGGTTGACAAGAACAAAGATGAC | |
| | GAGCTGACGGGTTTC | |
| 129 | ATGATCCCAGGTGGCCTGAGCGAAGCAAAACCAGCAACCCCAGAGATTCAAGA | 896 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCAGCGTGTTCATTTC | |
| | CCAGATTGGCCAGGTACGAACTACTACATCAAGGTTCGTGCGGGTGACAACAA | |
| | GTATATGCACCTGAAAGTGTTTAAGAGCCTGAACAAAGAAAACCAGGATGAAG | |
| | AAGATGAAGATTTGGTGCTGACGGGCTACCAGGTTGACAAGAACAAAGATGAC | |
| | GAGCTGACGGGTTTC | |
| 130 | ATGATCCCAGGTGGCCTGAGCGAAGCAAAACCAGCAACCCCAGAGATTCAAGA | 897 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCCACAGCCAGAATGG | |
| | AACGAATGGGGTGTTACGAACTACTACATCAAGGTTCGTGCGGGTGACAACAA | |
| | GTATATGCACCTGAAAGTGTTTAAGAGCCTGATCTTCTGGAACGAAAAACTGA | |
| | TCAGAGAAGATTTGGTGCTGACGGGCTACCAGGTTGACAAGAACAAAGATGAC | |
| | GAGCTGACGGGTTTC | |
| 131 | ATGATCCCAGGTGGCCTGAGCGAAGCAAAACCAGCAACCCCAGAGATTCAAGA | 898 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAACCAGTGGGAATGG | |
| | CCAGTTGATCTGGGTACGAACTACTACATCAAGGTTCGTGCGGGTGACAACAA | |
| | GTATATGCACCTGAAAGTGTTTAAGAGCCTGAAAGTTTACAGAACCCATCATA | |
| | TCTTCGAAGATTTGGTGCTGACGGGCTACCAGGTTGACAAGAACAAAGATGAC | |
| | GAGCTGACGGGTTTC | |
| 132 | ATGATCCCAGGTGGCCTGAGCGAAGCAAAACCAGCAACCCCAGAGATTCAAGA | 899 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGAACGTTACCATTTC | |
| | CCACAGTTCACCGGTACGAACTACTACATCAAGGTTCGTGCGGGTGACAACAA | |
| | GTATATGCACCTGAAAGTGTTTAAGAGCCTGAACCATGATCAGGTTAGATGGA | |
| | ACCTGGAAGATTTGGTGCTGACGGGCTACCAGGTTGACAAGAACAAAGATGAC | |
| | GAGCTGACGGGTTTC | |
| 133 | ATGATCCCAGGTGGCCTGAGCGAAGCAAAACCAGCAACCCCAGAGATTCAAGA | 900 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGTACTCTGAATCT | |
| | CGTGATTACAAAGGTACGAACTACTACATCAAGGTTCGTGCGGGTGACAACAA | |
| | GTATATGCACCTGAAAGTGTTTAAGAGCCTGCATGCATACAAAGGTTCTATCT | |
| | ACAGAGAAGATTTGGTGCTGACGGGCTACCAGGTTGACAAGAACAAAGATGAC | |
| | GAGCTGACGGGTTTC | |
| 134 | ATGATCCCAGGTGGCCTGAGCGAAGCAAAACCAGCAACCCCAGAGATTCAAGA | 901 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTTCTGGTTCGAAGAT | |
| | ATCCAGTGGCTGGGTACGAACTACTACATCAAGGTTCGTGCGGGTGACAACAA | |
| | GTATATGCACCTGAAAGTGTTTAAGAGCCTGAGATTCAGAGAAGATGTTGATA | |
| | TCAGAGAAGATTTGGTGCTGACGGGCTACCAGGTTGACAAGAACAAAGATGAC | |
| | GAGCTGACGGGTTTC | |
| 135 | ATGATCCCAGGTGGCCTGAGCGAAGCAAAACCAGCAACCCCAGAGATTCAAGA | 902 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGCAGTTTACCCAACC | |
| | TTCAAATACGGTACGAACTACTACATCAAGGTTCGTGCGGGTGACAACAAGTA | |
| | TATGCACCTGAAAGTGTTTAAGAGCCTGAAAGTTGAATACCTGGATTTCATCA | |
| | CCGAAGATTTGGTGCTGACGGGCTACCAGGTTGACAAGAACAAAGATGACGAG | |
| | CTGACGGGTTTC | |
| 136 | ATGATCCCAGGTGGCCTGAGCGAAGCAAAACCAGCAACCCCAGAGATTCAAGA | 903 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTCTGCGTTGGCAG | |
| | TGGGGTTACCTGGGTACGAACTACTACATCAAGGTTCGTGCGGGTGACAACAA | |
| | GTATATGCACCTGAAAGTGTTTAAGAGCCTGCAGCTGCTGTACGAAGATAACG | |
| | ATGAAGATTTGGTGCTGACGGGCTACCAGGTTGACAAGAACAAAGATGACGAG | |
| | CTGACGGGTTTC | |
| 137 | ATGATCCCAGGTGGCCTGAGCGAAGCAAAACCAGCAACCCCAGAGATTCAAGA | 904 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGTGGCCACTGCAG | |
| | CCAAAAAACTGGGGTACGAACTACTACATCAAGGTTCGTGCGGGTGACAACAA | |
| | GTATATGCACCTGAAAGTGTTTAAGAGCCTGGGTTGGAACTACCTGGAAGCAA | |
| | TCGCAGAAGATTTGGTGCTGACGGGCTACCAGGTTGACAAGAACAAAGATGAC | |
| | GAGCTGACGGGTTTC | |
| 138 | ATGATCCCAGGTGGCCTGAGCGAAGCAAAACCAGCAACCCCAGAGATTCAAGA | 905 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATAACGTTCGTCAT | |
| | GGTTGGCTGTCTGGTACGAACTACTACATCAAGGTTCGTGCGGGTGACAACAA | |
| | GTATATGCACCTGAAAGTGTTTAAGAGCCTGTTCGCACTGCTGTGGTCTGCAT | |
| | ACGGTGAAGATTTGGTGCTGACGGGCTACCAGGTTGACAAGAACAAAGATGAC | |
| | GAGCTGACGGGTTTC | |
| 139 | ATGATCCCAGGTGGCCTGAGCGAAGCAAAACCAGCAACCCCAGAGATTCAAGA | 906 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGGTCTGATCCAGCCA | |
| | CCATACGCACAGGGTACGAACTACTACATCAAGGTTCGTGCGGGTGACAACAA | |
| | GTATATGCACCTGAAAGTGTTTAAGAGCCTGGGTTGGCAGTACATCGATCTGT | |
| | TCCATGAAGATTTGGTGCTGACGGGCTACCAGGTTGACAAGAACAAAGATGAC | |
| | GAGCTGACGGGTTTC | |
| 140 | ATGATCCCAGGTGGCCTGAGCGAAGCAAAACCAGCAACCCCAGAGATTCAAGA | 907 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTCAGGTTGGTGTT | |
| | TGGGCATACGAAGGTACGAACTACTACATCAAGGTTCGTGCGGGTGACAACAA | |
| | GTATATGCACCTGAAAGTGTTTAAGAGCCTGGGTAGAACCGTTGAAGGTGGTA | |
| | GATGGGAAGATTTGGTGCTGACGGGCTACCAGGTTGACAAGAACAAAGATGAC | |
| | GAGCTGACGGGTTTC | |
| 141 | ATGATCCCAGGTGGCCTGAGCGAAGCAAAACCAGCAACCCCAGAGATTCAAGA | 908 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATCTGCATACCCTG | |
| | AAATCTGGTGTTGGTACGAACTACTACATCAAGGTTCGTGCGGGTGACAACAA | |
| | GTATATGCACCTGAAAGTGTTTAAGAGCCTGTACATCAAACATCATCTGTGGT | |
| | GGATCGAAGATTTGGTGCTGACGGGCTACCAGGTTGACAAGAACAAAGATGAC | |
| | GAGCTGACGGGTTTC | |
| 142 | ATGATCCCAGGTGGCCTGAGCGAAGCAAAACCAGCAACCCCAGAGATTCAAGA | 909 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTCTTGGTACAAACTG | |
| | TGGTCTTACTCTGGTACGAACTACTACATCAAGGTTCGTGCGGGTGACAACAA | |
| | GTATATGCACCTGAAAGTGTTTAAGAGCCTGTTCGAAGGTAGACATAAAGATC | |
| | CAGAAGAAGATTTGGTGCTGACGGGCTACCAGGTTGACAAGAACAAAGATGAC | |
| | GAGCTGACGGGTTTC | |
| 143 | ATGATCCCAGGTGGCCTGAGCGAAGCAAAACCAGCAACCCCAGAGATTCAAGA | 910 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATTGGCCATCTCAT | |
| | GCAAAATTCTGGGGTACGAACTACTACATCAAGGTTCGTGCGGGTGACAACAA | |
| | GTATATGCACCTGAAAGTGTTTAAGAGCCTGTTCCTGCCAGATAAAGATGATC | |
| | CATTCGAAGATTTGGTGCTGACGGGCTACCAGGTTGACAAGAACAAAGATGAC | |
| | GAGCTGACGGGTTTC | |
| 144 | ATGATCCCAGGTGGCCTGAGCGAAGCAAAACCAGCAACCCCAGAGATTCAAGA | 911 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGCATACCAGTCTCCA | |
| | CCTTACCATTTCGGTACGAACTACTACATCAAGGTTCGTGCGGGTGACAACAA | |
| | GTATATGCACCTGAAAGTGTTTAAGAGCCTGAGACAGATCGCAGGTTACATCG | |
| | CAATCGAAGATTTGGTGCTGACGGGCTACCAGGTTGACAAGAACAAAGATGAC | |
| | GAGCTGACGGGTTTC | |
| 145 | ATGATCCCAGGTGGCCTGAGCGAAGCAAAACCAGCAACCCCAGAGATTCAAGA | 912 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACAAAAACGTTGGT | |
| | CCATGGGTTAACGGTACGAACTACTACATCAAGGTTCGTGCGGGTGACAACAA | |
| | GTATATGCACCTGAAAGTGTTTAAGAGCCTGTACAGAACCAACAGAGGTCATG | |
| | AAAGAGAAGATTTGGTGCTGACGGGCTACCAGGTTGACAAGAACAAAGATGAC | |
| | GAGCTGACGGGTTTC | |
| 146 | ATGATCCCAGGTGGCCTGAGCGAAGCAAAACCAGCAACCCCAGAGATTCAAGA | 913 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTCTAAACGTTCTGCA | |
| | TGGCATTGGACCGGTACGAACTACTACATCAAGGTTCGTGCGGGTGACAACAA | |
| | GTATATGCACCTGAAAGTGTTTAAGAGCCTGTTCTGGAACGCACCAGATGATA | |
| | GACAGGAAGATTTGGTGCTGACGGGCTACCAGGTTGACAAGAACAAAGATGAC | |
| | GAGCTGACGGGTTTC | |
| 147 | ATGATCCCAGGTGGCCTGAGCGAAGCAAAACCAGCAACCCCAGAGATTCAAGA | 914 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTCATATCACCGTT | |
| | GAATGGGAAGGTACGAACTACTACATCAAGGTTCGTGCGGGTGACAACAAGTA | |
| | TATGCACCTGAAAGTGTTTAAGAGCCTGATCTACCAGAGACATACCCAGTCTA | |
| | GAGAAGATTTGGTGCTGACGGGCTACCAGGTTGACAAGAACAAAGATGACGAG | |
| | CTGACGGGTTTC | |
| 148 | ATGATCCCAGGTGGCCTGAGCGAAGCAAAACCAGCAACCCCAGAGATTCAAGA | 915 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTGATTACGATCCA | |
| | TTCCCATACACCGGTACGAACTACTACATCAAGGTTCGTGCGGGTGACAACAA | |
| | GTATATGCACCTGAAAGTGTTTAAGAGCCTGTGGAGAGGTCCAGGTCCATACG | |
| | CAATCGAAGATTTGGTGCTGACGGGCTACCAGGTTGACAAGAACAAAGATGAC | |
| | GAGCTGACGGGTTTC | |
| 149 | ATGATCCCAGGTGGCCTGAGCGAAGCAAAACCAGCAACCCCAGAGATTCAAGA | 916 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAAAACCCTGGGTGTT | |
| | TGGCATTGGTCTGGTACGAACTACTACATCAAGGTTCGTGCGGGTGACAACAA | |
| | GTATATGCACCTGAAAGTGTTTAAGAGCCTGTTCGAACAGCATGCAGAAAACA | |
| | TCGAAGAAGATTTGGTGCTGACGGGCTACCAGGTTGACAAGAACAAAGATGAC | |
| | GAGCTGACGGGTTTC | |
| 150 | ATGATCCCAGGTGGCCTGAGCGAAGCAAAACCAGCAACCCCAGAGATTCAAGA | 917 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTGAAGTTGGTCTG | |
| | TGGCGTTTCCAGGGTACGAACTACTACATCAAGGTTCGTGCGGGTGACAACAA | |
| | GTATATGCACCTGAAAGTGTTTAAGAGCCTGTACATCGGTTACGCAGGTGATG | |
| | CACAGGAAGATTTGGTGCTGACGGGCTACCAGGTTGACAAGAACAAAGATGAC | |
| | GAGCTGACGGGTTTC | |
| 151 | ATGATCCCAGGTGGCCTGAGCGAAGCAAAACCAGCAACCCCAGAGATTCAAGA | 918 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTGGGAAGAAGTTAAC | |
| | TGGCGTAACTGGGGTACGAACTACTACATCAAGGTTCGTGCGGGTGACAACAA | |
| | GTATATGCACCTGAAAGTGTTTAAGAGCCTGGTTCCAATCCATGATCATCTGA | |
| | GAAACGAAGATTTGGTGCTGACGGGCTACCAGGTTGACAAGAACAAAGATGAC | |
| | GAGCTGACGGGTTTC | |
| 152 | ATGATCCCAGGTGGCCTGAGCGAAGCAAAACCAGCAACCCCAGAGATTCAAGA | 919 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTGCACAGCTGCGT | |
| | CCATGGGTTGCAGGTACGAACTACTACATCAAGGTTCGTGCGGGTGACAACAA | |
| | GTATATGCACCTGAAAGTGTTTAAGAGCCTGGGTTGGAACGAAGAAGCAAGAC | |
| | CACCAGAAGATTTGGTGCTGACGGGCTACCAGGTTGACAAGAACAAAGATGAC | |
| | GAGCTGACGGGTTTC | |
| 153 | ATGATCCCAGGTGGCCTGAGCGAAGCAAAACCAGCAACCCCAGAGATTCAAGA | 920 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACCCATGGTCTTGG | |
| | AACGGTCATTACGGTACGAACTACTACATCAAGGTTCGTGCGGGTGACAACAA | |
| | GTATATGCACCTGAAAGTGTTTAAGAGCCTGATCAAATACCCATTCGCAAGAG | |
| | CAGAAGAAGATTTGGTGCTGACGGGCTACCAGGTTGACAAGAACAAAGATGAC | |
| | GAGCTGACGGGTTTC | |
| 154 | ATGATCCCAGGTGGCCTGAGCGAAGCAAAACCAGCAACCCCAGAGATTCAAGA | 92 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCCACAGATCGGTGCA | |
| | CCATACGTTAAAGGTACGAACTACTACATCAAGGTTCGTGCGGGTGACAACAA | |
| | GTATATGCACCTGAAAGTGTTTAAGAGCCTGGGTTGGAACTCTAACATCGGTG | |
| | TTCTGGAAGATTTGGTGCTGACGGGCTACCAGGTTGACAAGAACAAAGATGAC | |
| | GAGCTGACGGGTTTC | |
| 155 | ATGATCCCAGGTGGCCTGAGCGAAGCAAAACCAGCAACCCCAGAGATTCAAGA | 922 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTGGCGTACCGGTCAG | |
| | AAATGGCAGGTTGGTACGAACTACTACATCAAGGTTCGTGCGGGTGACAACAA | |
| | GTATATGCACCTGAAAGTGTTTAAGAGCCTGGCAGGTCATTACGAAGTTCATA | |
| | TCGGTGAAGATTTGGTGCTGACGGGCTACCAGGTTGACAAGAACAAAGATGAC | |
| | GAGCTGACGGGTTTC | |
| 156 | ATGATCCCAGGTGGCCTGAGCGAAGCAAAACCAGCAACCCCAGAGATTCAAGA | 923 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCCAGCAGCTGTAC | |
| | AAACAGACCTGGGGTACGAACTACTACATCAAGGTTCGTGCGGGTGACAACAA | |
| | GTATATGCACCTGAAAGTGTTTAAGAGCCTGGATTACCAGCATTGGCCATACG | |
| | ATTTCGAAGATTTGGTGCTGACGGGCTACCAGGTTGACAAGAACAAAGATGAC | |
| | GAGCTGACGGGTTTC | |
| 157 | ATGATCCCAGGTGGCCTGAGCGAAGCAAAACCAGCAACCCCAGAGATTCAAGA | 924 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCTACTACCCAATC | |
| | TGGGATTTCCATGGTACGAACTACTACATCAAGGTTCGTGCGGGTGACAACAA | |
| | GTATATGCACCTGAAAGTGTTTAAGAGCCTGTTCTGGGGTGATCTGAAAACCG | |
| | AAGATTTGGTGCTGACGGGCTACCAGGTTGACAAGAACAAAGATGACGAGCTG | |
| | ACGGGTTTC | |
| 158 | ATGATCCCAGGTGGCCTGAGCGAAGCAAAACCAGCAACCCCAGAGATTCAAGA | 925 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATAACGTTGCAAAC | |
| | ATCGTTCCACATGGTACGAACTACTACATCAAGGTTCGTGCGGGTGACAACAA | |
| | GTATATGCACCTGAAAGTGTTTAAGAGCCTGGGTTGGAACTACGAACATAAAT | |
| | GGCCAGAAGATTTGGTGCTGACGGGCTACCAGGTTGACAAGAACAAAGATGAC | |
| | GAGCTGACGGGTTTC | |
| 159 | ATGATCCCAGGTGGCCTGAGCGAAGCAAAACCAGCAACCCCAGAGATTCAAGA | 926 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCATGTTCATTGGGCA | |
| | TGGAACTACCTGGGTACGAACTACTACATCAAGGTTCGTGCGGGTGACAACAA | |
| | GTATATGCACCTGAAAGTGTTTAAGAGCCTGAGAAACTTCTACACCTTCCCAC | |
| | ATGAAGAAGATTTGGTGCTGACGGGCTACCAGGTTGACAAGAACAAAGATGAC | |
| | GAGCTGACGGGTTTC | |
| 160 | ATGATCCCAGGTGGCCTGAGCGAAGCAAAACCAGCAACCCCAGAGATTCAAGA | 927 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGGTCCATGGTACTTC | |
| | GGTTCTGCATGGGGTACGAACTACTACATCAAGGTTCGTGCGGGTGACAACAA | |
| | GTATATGCACCTGAAAGTGTTTAAGAGCCTGTGGGAAAACTTCCATAAACTGG | |
| | AAAAAGAAGATTTGGTGCTGACGGGCTACCAGGTTGACAAGAACAAAGATGAC | |
| | GAGCTGACGGGTTTC | |
| 161 | ATGATCCCAGGTGGCCTGAGCGAAGCAAAACCAGCAACCCCAGAGATTCAAGA | 928 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTCTGGAACGTCCA | |
| | CCATACGTTAAAGGTACGAACTACTACATCAAGGTTCGTGCGGGTGACAACAA | |
| | GTATATGCACCTGAAAGTGTTTAAGAGCCTGAACTGGGAAGGTGAACCATCTG | |
| | GTGTTGAAGATTTGGTGCTGACGGGCTACCAGGTTGACAAGAACAAAGATGAC | |
| | GAGCTGACGGGTTTC | |
| 162 | ATGATCCCAGGTGGCCTGAGCGAAGCAAAACCAGCAACCCCAGAGATTCAAGA | 929 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTTCACCTTCTCTTCT | |
| | ATCGGTTGGTGGGGTACGAACTACTACATCAAGGTTCGTGCGGGTGACAACAA | |
| | GTATATGCACCTGAAAGTGTTTAAGAGCCTGAGACAGCTGCTGGATAAACCAA | |
| | GATACGAAGATTTGGTGCTGACGGGCTACCAGGTTGACAAGAACAAAGATGAC | |
| | GAGCTGACGGGTTTC | |
| 163 | ATGATCCCAGGTGGCCTGAGCGAAGCAAAACCAGCAACCCCAGAGATTCAAGA | 930 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCTCTGCATCTGAA | |
| | ATCCGTTGGTACGGTACGAACTACTACATCAAGGTTCGTGCGGGTGACAACAA | |
| | GTATATGCACCTGAAAGTGTTTAAGAGCCTGAAAGCATTCGCAAACGATTACT | |
| | ACAAAGAAGATTTGGTGCTGACGGGCTACCAAGTTGACAAGAACAAAGATGAC | |
| | GAGCTGACGGGTTTC | |
| 164 | ATGATCCCAGGTGGCCTGAGCGAAGCAAAACCAGCAACCCCAGAGATTCAAGA | 931 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTATCTGGGGTTTC | |
| | TGGGCATTCCAGGGTACGAACTACTACATCAAGGTTCGTGCGGGTGACAACAA | |
| | GTATATGCACCTGAAAGTGTTTAAGAGCCTGGGTCATGAATCTGGTCAGAACC | |
| | TGTGGGAAGATTTGGTGCTGACGGGCTACCAGGTTGACAAGAACAAAGATGAC | |
| | GAGCTGACGGGTTTC | |
| 165 | ATGATCCCAGGTGGCCTGAGCGAAGCAAAACCAGCAACCCCAGAGATTCAAGA | 932 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAACGAAGCAGAATCT | |
| | TCTCGTTTCCTGGGTACGAACTACTACATCAAGGTTCGTGCGGGTGACAACAA | |
| | GTATATGCACCTGAAAGTGTTTAAGAGCCTGCATGATTTCACCCTGAAATGGA | |
| | AATTCGAAGATTTGGTGCTGACGGGCTACCAGGTTGACAAGAACAAAGATGAC | |
| | GAGCTGACGGGTTTC | |
| 166 | ATGATCCCAGGTGGCCTGAGCGAAGCAAAACCAGCAACCCCAGAGATTCAAGA | 933 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTGCAGGTCTGAAA | |
| | TGGCATTGGGAAGGTACGAACTACTACATCAAGGTTCGTGCGGGTGACAACAA | |
| | GTATATGCACCTGAAAGTGTTTAAGAGCCTGATCTACGCATCTCAGTACGATT | |
| | ACAGAGAAGATTTGGTGCTGACGGGCTACCAGGTTGACAAGAACAAAGATGAC | |
| | GAGCTGACGGGTTTC | |
| 167 | ATGATCCCAGGTGGCCTGAGCGAAGCAAAACCAGCAACCCCAGAGATTCAAGA | 934 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACCGTAACAAACAG | |
| | TGGTACAACGAAGGTACGAACTACTACATCAAGGTTCGTGCGGGTGACAACAA | |
| | GTATATGCACCTGAAAGTGTTTAAGAGCCTGAAAATCGGTTACCTGGAATTCG | |
| | TTCTGGAAGATTTGGTGCTGACGGGCTACCAGGTTGACAAGAACAAAGATGAC | |
| | GAGCTGACGGGTTTC | |
| 168 | ATGATCCCAGGTGGCCTGAGCGAAGCAAAACCAGCAACCCCAGAGATTCAAGA | 935 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTGGTTCGATACCTGG | |
| | CCAGTTCCAGAAGGTACGAACTACTACATCAAGGTTCGTGCGGGTGACAACAA | |
| | GTATATGCACCTGAAAGTGTTTAAGAGCCTGAACTGGAACGATCATCTGTCTA | |
| | CCTACGAAGATTTGGTGCTGACGGGCTACCAGGTTGACAAGAACAAAGATGAC | |
| | GAGCTGACGGGTTTC | |
| 169 | ATGATCCCAGGTGGCCTGAGCGAAGCAAAACCAGCAACCCCAGAGATTCAAGA | 936 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACGCAAACATCGGT | |
| | CCATGGCTGTACGGTACGAACTACTACATCAAGGTTCGTGCGGGTGACAACAA | |
| | GTATATGCACCTGAAAGTGTTTAAGAGCCTGTTCAAACATAGACAGCAGCTGC | |
| | ATTACACCGAAGATTTGGTGCTGACGGGCTACCAGGTTGACAAGAACAAAGAT | |
| | GACGAGCTGACGGGTTTC | |
| 170 | ATGATCCCAGGTGGCCTGAGCGAAGCAAAACCAGCAACCCCAGAGATTCAAGA | 937 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCATCAGTTCCAGGAA | |
| | ATCAAATGGTTCGGTACGAACTACTACATCAAGGTTCGTGCGGGTGACAACAA | |
| | GTATATGCACCTGAAAGTGTTTAAGAGCCTGTTCCATAGAACCCCAGGTTCTA | |
| | CCACCGAAGATTTGGTGCTGACGGGCTACCAGGTTGACAAGAACAAAGATGAC | |
| | GAGCTGACGGGTTTC | |
| 171 | ATGATCCCAGGTGGCCTGAGCGAAGCAAAACCAGCAACCCCAGAGATTCAAGA | 938 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCGATCATCAGAAC | |
| | CATCCAACCGAAGGTACGAACTACTACATCAAGGTTCGTGCGGGTGACAACAA | |
| | GTATATGCACCTGAAAGTGTTTAAGAGCCTGTGGCCATGGCTGCATTCTACCG | |
| | AACAGGAAGATTTGGTGCTGACGGGCTACCAGGTTGACAAGAACAAAGATGAC | |
| | GAGCTGACGGGTTTC | |
| 172 | ATGATCCCAGGTGGCCTGAGCGAAGCAAAACCAGCAACCCCAGAGATTCAAGA | 939 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTCTGTTTTCCAGTAC | |
| | ACCTACAAATTCGGTACGAACTACTACATCAAGGTTCGTGCGGGTGACAACAA | |
| | GTATATGCACCTGAAAGTGTTTAAGAGCCTGAACAACAAACCACCACATAACG | |
| | ATCTGGAAGATTTGGTGCTGACGGGCTACCAGGTTGACAAGAACAAAGATGAC | |
| | GAGCTGACGGGTTTC | |
| 173 | ATGATCCCAGGTGGCCTGAGCGAAGCAAAACCAGCAACCCCAGAGATTCAAGA | 940 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATTGGCCATCTCAT | |
| | GCAAAATTCTGGGGTACGAACTACTACATCAAGGTTCGTGCGGGTGACAACAA | |
| | GTATATGCACCTGAAAGTGTTTAAGAGCCTGTTCCTGCCAGATAAAGATGATC | |
| | CATTCGAAGATTTGGTGCTGACGGGCTACCAGGTTGACAAGAACAAAGATGAC | |
| | GAGCTGACGGGTTTC | |
| 174 | ATGATCCCAGGTGGCCTGAGCGAAGCAAAACCAGCAACCCCAGAGATTCAAGA | 941 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTCAGGTTACCATC | |
| | TGGCATTTCGAAGGTACGAACTACTACATCAAGGTTCGTGCGGGTGACAACAA | |
| | GTATATGCACCTGAAAGTGTTTAAGAGCCTGGGTAGAACCGAAGTTGAAGGTT | |
| | CTTTCGAAGATTTGGTGCTGACGGGCTACCAGGTTGACAAGAACAAAGATGAC | |
| | GAGCTGACGGGTTTC | |
| 175 | ATGATCCCAGGTGGCCTGAGCGAAGCAAAACCAGCAACCCCAGAGATTCAAGA | 942 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTGGCGTCGTTGGTTC | |
| | CCACAGTGGCCAGGTACGAACTACTACATCAAGGTTCGTGCGGGTGACAACAA | |
| | GTATATGCACCTGAAAGTGTTTAAGAGCCTGCCAGAAGCAGATGCAGATGATC | |
| | CAAAAGAAGATTTGGTGCTGACGGGCTACCAGGTTGACAAGAACAAAGATGAC | |
| | GAGCTGACGGGTTTC | |
| 176 | ATGATCCCAGGTGGCCTGAGCGAAGCAAAACCAGCAACCCCAGAGATTCAAGA | 943 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTTCTGGTACCCAGAA | |
| | ATCGGTTGGCTGGGTACGAACTACTACATCAAGGTTCGTGCGGGTGACAACAA | |
| | GTATATGCACCTGAAAGTGTTTAAGAGCCTGAGATCTAGATACCCAAACTACA | |
| | CCGCAGAAGATTTGGTGCTGACGGGCTACCAGGTTGACAAGAACAAAGATGAC | |
| | GAGCTGACGGGTTTC | |
| 177 | ATGATCCCAGGTGGCCTGAGCGAAGCAAAACCAGCAACCCCAGAGATTCAAGA | 944 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCTCTGAAGATTCT | |
| | TGGTGGCCACATGGTACGAACTACTACATCAAGGTTCGTGCGGGTGACAACAA | |
| | GTATATGCACCTGAAAGTGTTTAAGAGCCTGAACTGGTTCCATCAGCCATGGC | |
| | AGAGAGAAGATTTGGTGCTGACGGGCTACCAGGTTGACAAGAACAAAGATGAC | |
| | GAGCTGACGGGTTTC | |
| 178 | ATGATCCCAGGTGGCCTGAGCGAAGCAAAACCAGCAACCCCAGAGATTCAAGA | 945 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATCATCTGCCACAT | |
| | GCAAAATACTGGGGTACGAACTACTACATCAAGGTTCGTGCGGGTGACAACAA | |
| | GTATATGCACCTGAAAGTGTTTAAGAGCCTGTGGTACCAGCCAAGATGGAAAG | |
| | TTGAAGAAGATTTGGTGCTGACGGGCTACCAGGTTGACAAGAACAAAGATGAC | |
| | GAGCTGACGGGTTTC | |
| 179 | ATGATCCCAGGTGGCCTGAGCGAAGCAAAACCAGCAACCCCAGAGATTCAAGA | 946 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTTCAAAACCGATGAT | |
| | GCACGTTGGATCGGTACGAACTACTACATCAAGGTTCGTGCGGGTGACAACAA | |
| | GTATATGCACCTGAAAGTGTTTAAGAGCCTGTTCAACTACAACAAAACCGAAC | |
| | ATACCGAAGATTTGGTGCTGACGGGCTACCAGGTTGACAAGAACAAAGATGAC | |
| | GAGCTGACGGGTTTC | |
| 180 | ATGATCCCAGGTGGCCTGAGCGAAGCAAAACCAGCAACCCCAGAGATTCAAGA | 947 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGCAGGGTTCTCGT | |
| | CTGCCAATCGAAGGTACGAACTACTACATCAAGGTTCGTGCGGGTGACAACAA | |
| | GTATATGCACCTGAAAGTGTTTAAGAGCCTGGGTTGGCATAACGTTACCCTGC | |
| | CACCAGAAGATTTGGTGCTGACGGGCTACCAGGTTGACAAGAACAAAGATGAC | |
| | GAGCTGACGGGTTTC | |
| 181 | ATGATCCCAGGTGGCCTGAGCGAAGCAAAACCAGCAACCCCAGAGATTCAAGA | 948 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAAAGCATACAAAGTT | |
| | TTCAACTACATCGGTACGAACTACTACATCAAGGTTCGTGCGGGTGACAACAA | |
| | GTATATGCACCTGAAAGTGTTTAAGAGCCTGTTCAAATGGGATAACGGTATCT | |
| | CTGATGAAGATTTGGTGCTGACGGGCTACCAGGTTGACAAGAACAAAGATGAC | |
| | GAGCTGACGGGTTTC | |
| 182 | ATGATCCCAGGTGGCCTGAGCGAAGCAAAACCAGCAACCCCAGAGATTCAAGA | 949 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGAAAACCAACCCA | |
| | TCTCCATCTGATGGTACGAACTACTACATCAAGGTTCGTGCGGGTGACAACAA | |
| | GTATATGCACCTGAAAGTGTTTAAGAGCCTGGCAGTTTTCAGACAGAGACTGG | |
| | GTGTTGAAGATTTGGTGCTGACGGGCTACCAGGTTGACAAGAACAAAGATGAC | |
| | GAGCTGACGGGTTTC | |
| 183 | ATGATCCCAGGTGGCCTGAGCGAAGCAAAACCAGCAACCCCAGAGATTCAAGA | 950 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTCAGACCGATTCT | |
| | GTTAAATGGTGGGGTACGAACTACTACATCAAGGTTCGTGCGGGTGACAACAA | |
| | GTATATGCACCTGAAAGTGTTTAAGAGCCTGTGGTACAACAGATACTACAACG | |
| | ATTTCGAAGATTTGGTGCTGACGGGCTACCAGGTTGACAAGAACAAAGATGAC | |
| | GAGCTGACGGGTTTC | |
| 184 | ATGATCCCAGGTGGCCTGAGCGAAGCAAAACCAGCAACCCCAGAGATTCAAGA | 951 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCATCGTAAACATTTC | |
| | CATCAGTTCACCGGTACGAACTACTACATCAAGGTTCGTGCGGGTGACAACAA | |
| | GTATATGCACCTGAAAGTGTTTAAGAGCCTGTCTAACGATTACTACGAAACCT | |
| | ACCTGGAAGATTTGGTGCTGACGGGCTACCAGGTTGACAAGAACAAAGATGAC | |
| | GAGCTGACGGGTTTC | |
| 185 | ATGATCCCAGGTGGCCTGAGCGAAGCAAAACCAGCAACCCCAGAGATTCAAGA | 952 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTGTTCTGTTCGCA | |
| | TCTGGTTGGGAAGGTACGAACTACTACATCAAGGTTCGTGCGGGTGACAACAA | |
| | GTATATGCACCTGAAAGTGTTTAAGAGCCTGATCCAGTGGAGAGATCATAAAC | |
| | CAAGAGAAGATTTGGTGCTGACGGGCTACCAGGTTGACAAGAACAAAGATGAC | |
| | GAGCTGACGGGTTTC | |
| 186 | ATGATCCCAGGTGGCCTGAGCGAAGCAAAACCAGCAACCCCAGAGATTCAAGA | 953 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACGATAACGTTAAA | |
| | GAATACCCATGGGGTACGAACTACTACATCAAGGTTCGTGCGGGTGACAACAA | |
| | GTATATGCACCTGAAAGTGTTTAAGAGCCTGAAATGGTACGATCCAACCATCA | |
| | GACCAGAAGATTTGGTGCTGACGGGCTACCAAGTTGACAAGAACAAAGATGAC | |
| | GAGCTGACGGGTTTC | |
| 187 | ATGATCCCAGGTGGCCTGAGCGAAGCAAAACCAGCAACCCCAGAGATTCAAGA | 954 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCCAGGATTGGACC | |
| | GTTGGTTGGGAAGGTACGAACTACTACATCAAGGTTCGTGCGGGTGACAACAA | |
| | GTATATGCACCTGAAAGTGTTTAAGAGCCTGAGAATCGGTAGATCTACCAACC | |
| | TGTGGGAAGATTTGGTGCTGACGGGCTACCAGGTTGACAAGAACAAAGATGAC | |
| | GAGCTGACGGGTTTC | |
| 188 | ATGATCCCAGGTGGCCTGAGCGAAGCAAAACCAGCAACCCCAGAGATTCAAGA | 955 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCCACAGGAAAACGCA | |
| | CCATGGGTTCTGGGTACGAACTACTACATCAAGGTTCGTGCGGGTGACAACAA | |
| | GTATATGCACCTGAAAGTGTTTAAGAGCCTGAACTGGGATCAGCCAGATACCG | |
| | TTCTGGAAGATTTGGTGCTGACGGGCTACCAGGTTGACAAGAACAAAGATGAC | |
| | GAGCTGACGGGTTTC | |
| 189 | ATGATCCCAGGTGGCCTGAGCGAAGCAAAACCAGCAACCCCAGAGATTCAAGA | 956 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTTCCAGTCTACCGAT | |
| | TCTTTCTGGCATGGTACGAACTACTACATCAAGGTTCGTGCGGGTGACAACAA | |
| | GTATATGCACCTGAAAGTGTTTAAGAGCCTGTTCGTTGAAAACTGGCAGAAAA | |
| | GAACCGAAGATTTGGTGCTGACGGGCTACCAGGTTGACAAGAACAAAGATGAC | |
| | GAGCTGACGGGTTTC | |
| 190 | ATGATCCCAGGTGGCCTGAGCGAAGCAAAACCAGCAACCCCAGAGATTCAAGA | 957 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGGTGTTAAATCTACC | |
| | TTCCATTGGTCTGGTACGAACTACTACATCAAGGTTCGTGCGGGTGACAACAA | |
| | GTATATGCACCTGAAAGTGTTTAAGAGCCTGTTCAACAACCATCCACTGTCTC | |
| | CAGATGAAGATTTGGTGCTGACGGGCTACCAGGTTGACAAGAACAAAGATGAC | |
| | GAGCTGACGGGTTTC | |
| 191 | ATGATCCCAGGTGGCCTGAGCGAAGCAAAACCAGCAACCCCAGAGATTCAAGA | 958 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGCGTTACCATTTC | |
| | CCAGAATTCCATGGTACGAACTACTACATCAAGGTTCGTGCGGGTGACAACAA | |
| | GTATATGCACCTGAAAGTGTTTAAGAGCCTGAACCATCTGGCAATCCATGAAC | |
| | TGCCAGAAGATTTGGTGCTGACGGGCTACCAGGTTGACAAGAACAAAGATGAC | |
| | GAGCTGACGGGTTTC | |
| 192 | ATGATCCCAGGTGGCCTGAGCGAAGCAAAACCAGCAACCCCAGAGATTCAAGA | 959 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCACCGAAAACAACTCT | |
| | TGGTGGCCAATCGGTACGAACTACTACATCAAGGTTCGTGCGGGTGACAACAA | |
| | GTATATGCACCTGAAAGTGTTTAAGAGCCTGAACTGGTTCTCTGATGAAGCAC | |
| | CATACGAAGATTTGGTGCTGACGGGCTACCAGGTTGACAAGAACAAAGATGAC | |
| | GAGCTGACGGGTTTC | |
| 193 | ATGATCCCAGGTGGCCTGAGCGAAGCAAAACCAGCAACCCCAGAGATTCAAGA | 960 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGCGTCATCCAGTT | |
| | CAGGATGATGATGGTACGAACTACTACATCAAGGTTCGTGCGGGTGACAACAA | |
| | GTATATGCACCTGAAAGTGTTTAAGAGCCTGGCAGGTGTTAACAGATGGAAAT | |
| | GGATCGAAGATTTGGTGCTGACGGGCTACCAGGTTGACAAGAACAAAGATGAC | |
| | GAGCTGACGGGTTTC | |
| 194 | ATGATCCCAGGTGGCCTGAGCGAAGCAAAACCAGCAACCCCAGAGATTCAAGA | 961 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGAAGAACGTCGTATC | |
| | TGGAACTGGACCGGTACGAACTACTACATCAAGGTTCGTGCGGGTGACAACAA | |
| | GTATATGCACCTGAAAGTGTTTAAGAGCCTGTTCACCGCATCTGATACCCATC | |
| | CAGCAGAAGATTTGGTGCTGACGGGCTACCAGGTTGACAAGAACAAAGATGAC | |
| | GAGCTGACGGGTTTC | |
| 195 | ATGATCCCAGGTGGCCTGAGCGAAGCAAAACCAGCAACCCCAGAGATTCAAGA | 962 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTGGTGTTGGTCGT | |
| | GATTTCCCAAAAGGTACGAACTACTACATCAAGGTTCGTGCGGGTGACAACAA | |
| | GTATATGCACCTGAAAGTGTTTAAGAGCCTGTACATCCATTACCTGAAAGTTA | |
| | ACGAAGAAGATTTGGTGCTGACGGGCTACCAGGTTGACAAGAACAAAGATGAC | |
| | GAGCTGACGGGTTTC | |
| 196 | ATGATCCCAGGTGGCCTGAGCGAAGCAAAACCAGCAACCCCAGAGATTCAAGA | 963 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGCTGTGGACCCCA | |
| | CCATACGTTGATGGTACGAACTACTACATCAAGGTTCGTGCGGGTGACAACAA | |
| | GTATATGCACCTGAAAGTGTTTAAGAGCCTGGGTTGGAACGATGATCATTCTG | |
| | TTGGTGAAGATTTGGTGCTGACGGGCTACCAGGTTGACAAGAACAAAGATGAC | |
| | GAGCTGACGGGTTTC | |
| 197 | ATGATCCCAGGTGGCCTGAGCGAAGCAAAACCAGCAACCCCAGAGATTCAAGA | 964 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTGGAAACCACAGGAT | |
| | TCTGTTAACTGGGGTACGAACTACTACATCAAGGTTCGTGCGGGTGACAACAA | |
| | GTATATGCACCTGAAAGTGTTTAAGAGCCTGGATTGGGATGGTGCAGCAGGTG | |
| | ATGCAGAAGATTTGGTGCTGACGGGCTACCAGGTTGACAAGAACAAAGATGAC | |
| | GAGCTGACGGGTTTC | |
| 198 | ATGATCCCAGGTGGCCTGAGCGAAGCAAAACCAGCAACCCCAGAGATTCAAGA | 965 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTCTTCTCCACTGCAT | |
| | TGGGAACTGTGGCATTGGTCTGGTACGAACTACTACATCAAGGTTCGTGCGGG | |
| | TGACAACAAGTATATGCACCTGAAAGTGTTTAAGAGCCTGGAACTGAACAACA | |
| | AAGATCAGAACGATGAAGATTTGGTGCTGACGGGCTACCAGGTTGACAAGAAC | |
| | AAAGATGACGAGCTGACGGGTTTC | |
| 199 | ATGATCCCAGGTGGCCTGAGCGAAGCAAAACCAGCAACCCCAGAGATTCAAGA | 966 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTGATCATGAACTG | |
| | GTTAACCAGTGGGGTACGAACTACTACATCAAGGTTCGTGCGGGTGACAACAA | |
| | GTATATGCACCTGAAAGTGTTTAAGAGCCTGTTCGGTCAGCCAGTTCAGGGTA | |
| | ACACCGAAGATTTGGTGCTGACGGGCTACCAGGTTGACAAGAACAAAGATGAC | |
| | GAGCTGACGGGTTTC | |
| 200 | ATGATCCCAGGTGGCCTGAGCGAAGCAAAACCAGCAACCCCAGAGATTCAAGA | 967 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACGGTCCACATCAT | |
| | CAGACCTACGCAGGTACGAACTACTACATCAAGGTTCGTGCGGGTGACAACAA | |
| | GTATATGCACCTGAAAGTGTTTAAGAGCCTGTACGCACTGCATATCAACAGAT | |
| | GGCCAGAAGATTTGGTGCTGACGGGCTACCAGGTTGACAAGAACAAAGATGAC | |
| | GAGCTGACGGGTTTC | |
| 201 | ATGATCCCAGGTGGCCTGAGCGAAGCAAAACCAGCAACCCCAGAGATTCAAGA | 968 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCAGCGTGCACACTTC | |
| | AAACAGTGGTACGGTACGAACTACTACATCAAGGTTCGTGCGGGTGACAACAA | |
| | GTATATGCACCTGAAAGTGTTTAAGAGCCTGCCAGGTGAACCACATCATATCC | |
| | TGGATGAAGATTTGGTGCTGACGGGCTACCAGGTTGACAAGAACAAAGATGAC | |
| | GAGCTGACGGGTTTC | |
| 202 | ATGATCCCAGGTGGCCTGAGCGAAGCAAAACCAGCAACCCCAGAGATTCAAGA | 969 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCCGTCATGTTATC | |
| | TGGTCTTACTCTGGTACGAACTACTACATCAAGGTTCGTGCGGGTGACAACAA | |
| | GTATATGCACCTGAAAGTGTTTAAGAGCCTGTGGGAAAGAAACGCATCTCTGT | |
| | CTGAAGAAGATTTGGTGCTGACGGGCTACCAAGTTGACAAGAACAAAGATGAC | |
| | GAGCTGACGGGTTTC | |
| 203 | ATGATCCCAGGTGGCCTGAGCGAAGCAAAACCAGCAACCCCAGAGATTCAAGA | 970 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACCATGGTCCACCA | |
| | AAAGAATGGCTGGGTACGAACTACTACATCAAGGTTCGTGCGGGTGACAACAA | |
| | GTATATGCACCTGAAAGTGTTTAAGAGCCTGTTCTTCTGGAAAGGTAACGGTT | |
| | ACAGAGAAGATTTGGTGCTGACGGGCTACCAGGTTGACAAGAACAAAGATGAC | |
| | GAGCTGACGGGTTTC | |
| 204 | ATGATCCCAGGTGGCCTGAGCGAAGCAAAACCAGCAACCCCAGAGATTCAAGA | 971 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTCTCTGCGTCCACAT | |
| | GAACAGCTGTACGGTACGAACTACTACATCAAGGTTCGTGCGGGTGACAACAA | |
| | GTATATGCACCTGAAAGTGTTTAAGAGCCTGCATCTGAAACTGTGGAAAATCC | |
| | CAAACGAAGATTTGGTGCTGACGGGCTACCAGGTTGACAAGAACAAAGATGAC | |
| | GAGCTGACGGGTTTC | |
| 205 | ATGATCCCAGGTGGCCTGAGCGAAGCAAAACCAGCAACCCCAGAGATTCAAGA | 972 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTTTCCTGTCTGAA | |
| | ATCCATTGGTACGGTACGAACTACTACATCAAGGTTCGTGCGGGTGACAACAA | |
| | GTATATGCACCTGAAAGTGTTTAAGAGCCTGAGAATCAACAACTCTGGTCTGT | |
| | CTAGAGAAGATTTGGTGCTGACGGGCTACCAGGTTGACAAGAACAAAGATGAC | |
| | GAGCTGACGGGTTTC | |
| 206 | ATGATCCCAGGTGGCCTGAGCGAAGCAAAACCAGCAACCCCAGAGATTCAAGA | 973 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTGCAGATGTTAAC | |
| | GTTATCTGGTGGGGTACGAACTACTACATCAAGGTTCGTGCGGGTGACAACAA | |
| | GTATATGCACCTGAAAGTGTTTAAGAGCCTGCCAGTTAAATACAAAGAAATCC | |
| | TGCTGGAAGATTTGGTGCTGACGGGCTACCAGGTTGACAAGAACAAAGATGAC | |
| | GAGCTGACGGGTTTC | |
| 207 | ATGATCCCAGGTGGCCTGAGCGAAGCAAAACCAGCAACCCCAGAGATTCAAGA | 974 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATACCAAAACCCCA | |
| | TGGCTGTGGACCGGTACGAACTACTACATCAAGGTTCGTGCGGGTGACAACAA | |
| | GTATATGCACCTGAAAGTGTTTAAGAGCCTGTTCCTGAAAATCCTGGCAGAAG | |
| | ATGATGAAGATTTGGTGCTGACGGGCTACCAGGTTGACAAGAACAAAGATGAC | |
| | GAGCTGACGGGTTTC | |
| 208 | ATGATCCCAGGTGGCCTGAGCGAAGCAAAACCAGCAACCCCAGAGATTCAAGA | 975 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCAGTGGAAAAAAACC | |
| | CAGTACTTCTTCGGTACGAACTACTACATCAAGGTTCGTGCGGGTGACAACAA | |
| | GTATATGCACCTGAAAGTGTTTAAGAGCCTGAGAATCGGTTACCTGGAATTCG | |
| | AAATCGAAGATTTGGTGCTGACGGGCTACCAGGTTGACAAGAACAAAGATGAC | |
| | GAGCTGACGGGTTTC | |
| 209 | ATGATCCCAGGTGGCCTGAGCGAAGCAAAACCAGCAACCCCAGAGATTCAAGA | 976 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCGCAAAAGAAGAA | |
| | AACCATTGGCGTGGTACGAACTACTACATCAAGGTTCGTGCGGGTGACAACAA | |
| | GTATATGCACCTGAAAGTGTTTAAGAGCCTGCATTACTACCAGGATGTTCTGT | |
| | ACAGAGAAGATTTGGTGCTGACGGGCTACCAGGTTGACAAGAACAAAGATGAC | |
| | GAGCTGACGGGTTTC | |
| 210 | ATGATCCCAGGTGGCCTGAGCGAAGCAAAACCAGCAACCCCAGAGATTCAAGA | 977 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTGGGAATACGCAGGT | |
| | GATTACCTGGAAGGTACGAACTACTACATCAAGGTTCGTGCGGGTGACAACAA | |
| | GTATATGCACCTGAAAGTGTTTAAGAGCCTGTTCCATGGTATCTGGTGGGCAA | |
| | GAGTTGAAGATTTGGTGCTGACGGGCTACCAGGTTGACAAGAACAAAGATGAC | |
| | GAGCTGACGGGTTTC | |
| 211 | ATGATCCCAGGTGGCCTGAGCGAAGCAAAACCAGCAACCCCAGAGATTCAAGA | 978 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCACCCATCCAGAAGAT | |
| | GCATACTCTTGGGGTACGAACTACTACATCAAGGTTCGTGCGGGTGACAACAA | |
| | GTATATGCACCTGAAAGTGTTTAAGAGCCTGCTGAAACCAGGTGGTCTGCTGG | |
| | ATTTCGAAGATTTGGTGCTGACGGGCTACCAGGTTGACAAGAACAAAGATGAC | |
| | GAGCTGACGGGTTTC | |
| 212 | ATGATCCCAGGTGGCCTGAGCGAAGCAAAACCAGCAACCCCAGAGATTCAAGA | 979 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACGCATTCATCACC | |
| | CCAGTTACCCATGGTACGAACTACTACATCAAGGTTCGTGCGGGTGACAACAA | |
| | GTATATGCACCTGAAAGTGTTTAAGAGCCTGTGGCTGAACAACTTCGGTAAAA | |
| | ACTTCGAAGATTTGGTGCTGACGGGCTACCAGGTTGACAAGAACAAAGATGAC | |
| | GAGCTGACGGGTTTC | |
| 213 | ATGATCCCAGGTGGCCTGAGCGAAGCAAAACCAGCAACCCCAGAGATTCAAGA | 980 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAAACAGCATACCAAA | |
| | GAATGGGCACCAGGTACGAACTACTACATCAAGGTTCGTGCGGGTGACAACAA | |
| | GTATATGCACCTGAAAGTGTTTAAGAGCCTGTGGAACCATCTGTACCATTACC | |
| | CATTCGAAGATTTGGTGCTGACGGGCTACCAGGTTGACAAGAACAAAGATGAC | |
| | GAGCTGACGGGTTTC | |
| 214 | ATGATCCCAGGTGGCCTGAGCGAAGCAGAACCAGCAACCCCAGAGATTCAAGA | 981 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCAAAATCCATCAT | |
| | GGTTCTTGGGAAGGTACGAACTACTACATCAAGGTTCGTGCGGGTGACAACAA | |
| | GTATATGCACCTGAAAGTGTTTAAGAGCCTGCTGTTCTGGCAGAAACAGTTCT | |
| | ACAGAGAAGATTTGGTGCTGACGGGCTACCAGGTTGACAAGAACAAAGATGAC | |
| | GAGCTGACGGGTTTC | |
| 215 | ATGATCCCAGGTGGCCTGAGCGAAGCAAAACCAGCAACCCCAGAGATTCAAGA | 982 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTCGTGTTTTCTTC | |
| | CCAGAATTCCCAGGTACGAACTACTACATCAAGGTTCGTGCGGGTGACAACAA | |
| | GTATATGCACCTGAAAGTGTTTAAGAGCCTGGAACAGCCAATCGAAGGTGATC | |
| | TGTGGGAAGATTTGGTGCTGACGGGCTACCAGGTTGACAAGAACAAAGATGAC | |
| | GAGCTGACGGGTTTC | |
| 216 | ATGATCCCAGGTGGCCTGAGCGAAGCAAAACCAGCAACCCCAGAGATTCAAGA | 983 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTTCACCGAAGTTAAA | |
| | CCATTCGTTGAAGGTACGAACTACTACATCAAGGTTCGTGCGGGTGACAACAA | |
| | GTATATGCACCTGAAAGTGTTTAAGAGCCTGTCTTGGAACGCACCATTCCCAG | |
| | CAGGTGAAGATTTGGTGCTGACGGGCTACCAGGTTGACAAGAACAAAGATGAC | |
| | GAGCTGACGGGTTTC | |
| 217 | ATGATCCCAGGTGGCCTGAGCGAAGCAAAACCAGCAACCCCAGAGATTCAAGA | 984 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAAAAAATACGTTATC | |
| | TTCTTCTACGAAGGTACGAACTACTACATCAAGGTTCGTGCGGGTGACAACAA | |
| | GTATATGCACCTGAAAGTGTTTAAGAGCCTGTTCGTTAACAAAGGTGGTTCTG | |
| | ATGATGAAGATTTGGTGCTGACGGGCTACCAGGTTGACAAGAACAAAGATGAC | |
| | GAGCTGACGGGTTTC | |
| 218 | ATGATCCCAGGTGGCCTGAGCGAAGCAAAACCAGCAACCCCAGAGATTCAAGA | 985 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTGGCGTGTTTACTTC | |
| | CCAGATTTCAAAGGTACGAACTACTACATCAAGGTTCGTGCGGGTGACAACAA | |
| | GTATATGCACCTGAAAGTGTTTAAGAGCCTGGATGAAGTTGATGAAATCCCAC | |
| | ATTTCGAAGATTTGGTGCTGACGGGCTACCAGGTTGACAAGAACAAAGATGAC | |
| | GAGCTGACGGGTTTC | |
| 219 | ATGATCCCAGGTGGCCTGAGCGAAGCAAAACCAGCAACCCCAGAGATTCAAGA | 986 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCCACTGACCATCCCA | |
| | CCATTCGTTGAAGGTACGAACTACTACATCAAGGTTCGTGCGGGTGACAACAA | |
| | GTATATGCACCTGAAAGTGTTTAAGAGCCTGGGTTGGCAGCAGGTTAACGATG | |
| | TTAAAGAAGATTTGGTGCTGACGGGCTACCAGGTTGACAAGAACAAAGATGAC | |
| | GAGCTGACGGGTTTC | |
| 220 | ATGATCCCAGGTGGCCTGAGCGAAGCAAAACCAGCAACCCCAGAGATTCAAGA | 987 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCAACCGTTGGAACATC | |
| | CGTGAATTCGAAGGTACGAACTACTACATCAAGGTTCGTGCGGGTGACAACAA | |
| | GTATATGCACCTGAAAGTGTTTAAGAGCCTGATCAACGTTCCAGTTACCGAAC | |
| | TGCTGGAAGATTTGGTGCTGACGGGCTACCAGGTTGACAAGAACAAAGATGAC | |
| | GAGCTGACGGGTTTC | |
| 221 | ATGATCCCAGGTGGCCTGAGCGAAGCAAAACCAGCAACCCCAGAGATTCAAGA | 988 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCCAGGATGTTGAT | |
| | TGGCGTCTGTGGGGTACGAACTACTACATCAAGGTTCGTGCGGGTGACAACAA | |
| | GTATATGCACCTGAAAGTGTTTAAGAGCCTGGTTCCATCTGATTGGTCTAAAA | |
| | CCATCGAAGATTTGGTGCTGACGGGCTACCAGGTTGACAAGAACAAAGATGAC | |
| | GAGCTGACGGGTTTC | |
| 222 | ATGATCCCAGGTGGCCTGAGCGAAGCAGAACCAGCAACCCCAGAGATTCAAGA | 989 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCAGCGTGATCATTTC | |
| | CCAGAATTCCCAGGTACGAACTACTACATCAAGGTTCGTGCGGGTGACAACAA | |
| | GTATATGCACCTGAAAGTGTTTAAGAGCCTGGGTCATGAACTGTACGCACTGT | |
| | ACGCAGAAGATTTGGTGCTGACGGGCTACCAGGTTGACAAGAACAAAGATGAC | |
| | GAGCTGACGGGTTTC | |
| 223 | ATGATCCCAGGTGGCCTGAGCGAAGCAAAACCAGCAACCCCAGAGATTCAAGA | 990 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTTCCCATGGGATCAG | |
| | GAAAACTACTACGGTACGAACTACTACATCAAGGTTCGTGCGGGTGACAACAA | |
| | GTATATGCACCTGAAAGTGTTTAAGAGCCTGATCAAAGAACCATTCATCAAAA | |
| | ACGTTGAAGATTTGGTGCTGACGGGCTACCAGGTTGACAAGAACAAAGATGAC | |
| | GAGCTGACGGGTTTC | |
| 224 | ATGATCCCAGGTGGCCTGAGCGAAGCAAAACCAGCAACCCCAGAGATTCAAGA | 991 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCACCCGTCGTCATTTC | |
| | GTTCAGTTCACCGGTACGAACTACTACATCAAGGTTCGTGCGGGTGACAACAA | |
| | GTATATGCACCTGAAAGTGTTTAAGAGCCTGGATGAACCAGCAACCCCAACCT | |
| | ACCTGGAAGATTTGGTGCTGACGGGCTACCAGGTTGACAAGAACAAAGATGAC | |
| | GAGCTGACGGGTTTC | |
| 225 | ATGATCCCAGGTGGCCTGAGCGAAGCAAAACCAGCAACCCCAGAGATTCAAGA | 992 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGATCTGCATACCCCA | |
| | CATTACGCACCAGGTACGAACTACTACATCAAGGTTCGTGCGGGTGACAACAA | |
| | GTATATGCACCTGAAAGTGTTTAAGAGCCTGTGGAACACCCTGCTGGAATTCG | |
| | GTGGTGAAGATTTGGTGCTGACGGGCTACCAGGTTGACAAGAACAAAGATGAC | |
| | GAGCTGACGGGTTTC | |
| 226 | ATGATCCCAGGTGGCCTGAGCGAAGCAAAACCAGCAACCCCAGAGATTCAAGA | 993 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACAAACATGATGAT | |
| | GAATACGGTTTCGGTACGAACTACTACATCAAGGTTCGTGCGGGTGACAACAA | |
| | GTATATGCACCTGAAAGTGTTTAAGAGCCTGAAATGGACCAACAGAGCAAACA | |
| | AACCAGAAGATTTGGTGCTGACGGGCTACCAGGTTGACAAGAACAAAGATGAC | |
| | GAGCTGACGGGTTTC | |
| 227 | ATGATCCCAGGTGGCCTGAGCGAAGCAAAACCAGCAACCCCAGAGATTCAAGA | 994 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTACGTTCGTTGGCAT | |
| | GAATACGGTTTCGGTACGAACTACTACATCAAGGTTCGTGCGGGTGACAACAA | |
| | GTATATGCACCTGAAAGTGTTTAAGAGCCTGTCTAACCAGCTGATCCCACAGC | |
| | CAATCGAAGATTTGGTGCTGACGGGCTACCAGGTTGACAAGAACAAAGATGAC | |
| | GAGCTGACGGGTTTC | |
| 228 | ATGATCCCAGGTGGCCTGAGCGAAGCAAAACCAGCAACCCCAGAGATTCAAGA | 995 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTTGGACCTCTGAT | |
| | GATGCATGGCAGGGTACGAACTACTACATCAAGGTTCGTGCGGGTGACAACAA | |
| | GTATATGCACCTGAAAGTGTTTAAGAGCCTGTTCAAATTCGAAATCTACAAAC | |
| | ATACCGAAGATTTGGTGCTGACGGGCTACCAGGTTGACAAGAACAAAGATGAC | |
| | GAGCTGACGGGTTTC | |
| 229 | ATGATCCCAGGTGGCCTGAGCGAAGCAAAACCAGCAACCCCAGAGATTCAAGA | 996 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCATTCTTCTTCTAAA | |
| | CCATACATCCATGGTACGAACTACTACATCAAGGTTCGTGCGGGTGACAACAA | |
| | GTATATGCACCTGAAAGTGTTTAAGAGCCTGAACTGGAACAGACCATACAACG | |
| | ATCTGGAAGATTTGGTGCTGACGGGCTACCAAGTTGACAAGAACAAAGATGAC | |
| | GAGCTGACGGGTTTC | |
| 230 | ATGATCCCAGGTGGCCTGAGCGAAGCAAAACCAGCAACCCCAGAGATTCAAGA | 997 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCATGTTTCTAACTTC | |
| | ATCATCCCACATGGTACGAACTACTACATCAAGGTTCGTGCGGGTGACAACAA | |
| | GTATATGCACCTGAAAGTGTTTAAGAGCCTGAACTGGGAAGGTAACGATCTGG | |
| | CAACCGAAGATTTGGTGCTGACGGGCTACCAGGTTGACAAGAACAAAGATGAC | |
| | GAGCTGACGGGTTTC | |
| 231 | ATGATCCCAGGTGGCCTGAGCGAAGCAAAACCAGCAACCCCAGAGATTCAAGA | 998 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGCACGTTGGCATTGG | |
| | TTCCATCATTTCGGTACGAACTACTACATCAAGGTTCGTGCGGGTGACAACAA | |
| | GTATATGCACCTGAAAGTGTTTAAGAGCCTGAACCCAATCAACGGTCAGGAAA | |
| | CCTGGGAAGATTTGGTGCTGACGGGCTACCAGGTTGACAAGAACAAAGATGAC | |
| | GAGCTGACGGGTTTC | |
| 232 | ATGATCCCAGGTGGCCTGAGCGAAGCAAAACCAGCAACCCCAGAGATTCAAGA | 999 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCACCAAACATTCTGCA | |
| | ACCTTCTTCCATGGTACGAACTACTACATCAAGGTTCGTGCGGGTGACAACAA | |
| | GTATATGCACCTGAAAGTGTTTAAGAGCCTGATCGGTAACGGTCAGTACTGGT | |
| | ACAGAGAAGATTTGGTGCTGACGGGCTACCAGGTTGACAAGAACAAAGATGAC | |
| | GAGCTGACGGGTTTC | |
| 233 | ATGATCCCAGGTGGCCTGAGCGAAGCAAAACCAGCAACCCCAGAGATTCAAGA | 1000 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGCGTTACCATTTC | |
| | CAGCATTTCCCAGGTACGAACTACTACATCAAGGTTCGTGCGGGTGACAACAA | |
| | GTATATGCACCTGAAAGTGTTTAAGAGCCTGGATGGTCAGGGTGCACTGTCTC | |
| | TGCCAGAAGATTTGGTGCTGACGGGCTACCAGGTTGACAAGAACAAAGATGAC | |
| | GAGCTGACGGGTTTC | |
| 234 | ATGATCCCAGGTGGCCTGAGCGAAGCAAAACCAGCAACCCCAGAGATTCAAGA | 1001 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTAACTCTGGTGTT | |
| | TGGGAATTCGAAGGTACGAACTACTACATCAAGGTTCGTGCGGGTGACAACAA | |
| | GTATATGCACCTGAAAGTGTTTAAGAGCCTGGGTAAATACAAAACCAACTGGT | |
| | ACTTCGAAGATTTGGTGCTGACGGGCTACCAGGTTGACAAGAACAAAGATGAC | |
| | GAGCTGACGGGTTTC | |
| 235 | ATGATCCCAGGTGGCCTGAGCGAAGCAAAACCAGCAACCCCAGAGATTCAAGA | 1002 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTGGAACCCACCATCT | |
| | CTGCCAATCTCTGGTACGAACTACTACATCAAGGTTCGTGCGGGTGACAACAA | |
| | GTATATGCACCTGAAAGTGTTTAAGAGCCTGGCATGGGATAACACCGCAAAAC | |
| | ATGGTGAAGATTTGGTGCTGACGGGCTACCAGGTTGACAAGAACAAAGATGAC | |
| | GAGCTGACGGGTTTC | |
| 236 | ATGATCCCAGGTGGCCTGAGCGAAGCAAAACCAGCAACCCCAGAGATTCAAGA | 1003 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTGATGGTACCTTC | |
| | TGGGATTTCTGGGGTACGAACTACTACATCAAGGTTCGTGCGGGTGACAACAA | |
| | GTATATGCACCTGAAAGTGTTTAAGAGCCTGTCTCAGGATTACAACGGTGGTC | |
| | AGGCAGAAGATTTGGTGCTGACGGGCTACCAGGTTGACAAGAACAAAGATGAC | |
| | GAGCTGACGGGTTTC | |
| 237 | ATGATCCCAGGTGGCCTGAGCGAAGCAAAACCAGCAACCCCAGAGATTCAAGA | 1004 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTGCAGGTCTGAAA | |
| | TGGCATTGGGAAGGTACGAACTACTACATCAAGGTTCGTGCGGGTGACAACAA | |
| | GTATATGCACCTGAAAGTGTTTAAGAGCCTGATCTACGCATCTCAGTACGATT | |
| | ACAGAGAAGATTTGGTGCTGACGGGCTACCAGGTTGACAAGAACAAAGATGAC | |
| | GAGCTGACGGGTTTC | |
| 238 | ATGATCCCAGGTGGCCTGAGCGAAGCAAAACCAGCAACCCCAGAGATTCAAGA | 1005 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTCATAAAGGTCTG | |
| | TGGCAGTTCGAAGGTACGAACTACTACATCAAGGTTCGTGCGGGTGACAACAA | |
| | GTATATGCACCTGAAAGTGTTTAAGAGCCTGTCTCATCAGCCATACAGAGCAA | |
| | CCATCGAAGATTTGGTGCTGACGGGCTACCAGGTTGACAAGAACAAAGATGAC | |
| | GAGCTGACGGGTTTC | |
| 239 | ATGATCCCAGGTGGCCTGAGCGAAGCAAAACCAGCAACCCCAGAGATTCAAGA | 1006 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGACCATCCCACCA | |
| | ACCGCATTCTCTGGTACGAACTACTACATCAAGGTTCGTGCGGGTGACAACAA | |
| | GTATATGCACCTGAAAGTGTTTAAGAGCCTGGGTTGGAACCTGCATGAAGTTG | |
| | CACTGGAAGATTTGGTGCTGACGGGCTACCAGGTTGACAAGAACAAAGATGAC | |
| | GAGCTGACGGGTTTC | |
| 240 | ATGATCCCAGGTGGCCTGAGCGAAGCAAAACCAGCAACCCCAGAGATTCAAGA | 1007 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGCTGTACCAGGCA | |
| | CCATTCGTTCAGGGTACGAACTACTACATCAAGGTTCGTGCGGGTGACAACAA | |
| | GTATATGCACCTGAAAGTGTTTAAGAGCCTGGGTTGGGATCTGGAATACTCTC | |
| | AGAAAGAAGATTTGGTGCTGACGGGCTACCAGGTTGACAAGAACAAAGATGAC | |
| | GAGCTGACGGGTTTC | |
| 241 | ATGATCCCAGGTGGCCTGAGCGAAGCAAAACCAGCAACCCCAGAGATTCAAGA | 1008 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTCTCGTCTGCATTTC | |
| | CCAAACTTCCAGGGTACGAACTACTACATCAAGGTTCGTGCGGGTGACAACAA | |
| | GTATATGCACCTGAAAGTGTTTAAGAGCCTGAACCTGGAACATCCAACCGATG | |
| | AAACCGAAGATTTGGTGCTGACGGGCTACCAGGTTGACAAGAACAAAGATGAC | |
| | GAGCTGACGGGTTTC | |
| 242 | ATGATCCCAGGTGGCCTGAGCGAAGCAAAACCAGCAACCCCAGAGATTCAAGA | 1009 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGTTGAATGGTCTGAA | |
| | TCTGGTTACGATGGTACGAACTACTACATCAAGGTTCGTGCGGGTGACAACAA | |
| | GTATATGCACCTGAAAGTGTTTAAGAGCCTGAGACTGAAATCTAACGTTAACA | |
| | TCGCAGAAGATTTGGTGCTGACGGGCTACCAGGTTGACAAGAACAAAGATGAC | |
| | GAGCTGACGGGTTTC | |
| 243 | ATGATCCCAGGTGGCCTGAGCGAAGCAAAACCAGCAACCCCAGAGATTCAAGA | 1010 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTGGAAACAGATCACC | |
| | CTGCCAGTTGATGGTACGAACTACTACATCAAGGTTCGTGCGGGTGACAACAA | |
| | GTATATGCACCTGAAAGTGTTTAAGAGCCTGCATTGGGATGAATACGAATACG | |
| | ATGAAGAAGATTTGGTGCTGACGGGCTACCAGGTTGACAAGAACAAAGATGAC | |
| | GAGCTGACGGGTTTC | |
| 244 | ATGATCCCAGGTGGCCTGAGCGAAGCAAAACCAGCAACCCCAGAGATTCAAGA | 1011 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCTGGGATTCTGAAGCA | |
| | CTGCCAATCGATGGTACGAACTACTACATCAAGGTTCGTGCGGGTGACAACAA | |
| | GTATATGCACCTGAAAGTGTTTAAGAGCCTGAGATGGTGGCCATGGGATGCAG | |
| | TTGGTGAAGATTTGGTGCTGACGGGCTACCAGGTTGACAAGAACAAAGATGAC | |
| | GAGCTGACGGGTTTC | |
| 245 | ATGATCCCAGGTGGCCTGAGCGAAGCAAAACCAGCAACCCCAGAGATTCAAGA | 1012 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGGTTACCTGGAACTG | |
| | TGGAAATGGTCTGGTACGAACTACTACATCAAGGTTCGTGCGGGTGACAACAA | |
| | GTATATGCACCTGAAAGTGTTTAAGAGCCTGTACAACCTGCATCATCCACATA | |
| | AAGATGAAGATTTGGTGCTGACGGGCTACCAGGTTGACAAGAACAAAGATGAC | |
| | GAGCTGACGGGTTTC | |
| 246 | ATGATCCCAGGTGGCCTGAGCGAAGCAAAACCAGCAACCCCAGAGATTCAAGA | 1013 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGAACGTTTCCATTTC | |
| | CCACAGTTCCCAGGTACGAACTACTACATCAAGGTTCGTGCGGGTGACAACAA | |
| | GTATATGCACCTGAAAGTGTTTAAGAGCCTGCCACATCTGGATTACATCTGGA | |
| | ACTGGGAAGATTTGGTGCTGACGGGCTACCAGGTTGACAAGAACAAAGATGAC | |
| | GAGCTGACGGGTTTC | |
| 247 | ATGATCCCAGGTGGCCTGAGCGAAGCAAAACCAGCAACCCCAGAGATTCAAGA | 1014 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTCGTGAACATTTC | |
| | CCACGTTGGCCAGGTACGAACTACTACATCAAGGTTCGTGCGGGTGACAACAA | |
| | GTATATGCACCTGAAAGTGTTTAAGAGCCTGGATCTGCTGCTGAGAAAAGAAG | |
| | AAGTTGAAGATTTGGTGCTGACGGGCTACCAGGTTGACAAGAACAAAGATGAC | |
| | GAGCTGACGGGTTTC | |
| 248 | ATGATCCCAGGTGGCCTGAGCGAAGCAAAACCAGCAACCCCAGAGATTCAAGA | 1015 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCTGCAGCTGCCACCA | |
| | CGTCCACATGATGGTACGAACTACTACATCAAGGTTCGTGCGGGTGACAACAA | |
| | GTATATGCACCTGAAAGTGTTTAAGAGCCTGAACTGGGAACCAGCACAGCATC | |
| | CAGTTGAAGATTTGGTGCTGACGGGCTACCAGGTTGACAAGAACAAAGATGAC | |
| | GAGCTGACGGGTTTC | |
| 249 | ATGATCCCAGGTGGCCTGAGCGAAGCAAAACCAGCAACCCCAGAGATTCAAGA | 1016 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCTACTACCCAATC | |
| | TGGGGTTTCCAGGGTACGAACTACTACATCAAGGTTCGTGCGGGTGACAACAA | |
| | GTATATGCACCTGAAAGTGTTTAAGAGCCTGAGATGGTCTAAAGATGGTGCAA | |
| | CCGCAGAAGATTTGGTGCTGACGGGCTACCAGGTTGACAAGAACAAAGATGAC | |
| | GAGCTGACGGGTTTC | |
| 250 | ATGATCCCAGGTGGCCTGAGCGAAGCAAAACCAGCAACCCCAGAGATTCAAGA | 1017 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCCGTGATCCAGATGTT | |
| | TTCGTTTTCTCTGGTACGAACTACTACATCAAGGTTCGTGCGGGTGACAACAA | |
| | GTATATGCACCTGAAAGTGTTTAAGAGCCTGTGGTACTACAACGATAACCAGT | |
| | CTGGTGAAGATTTGGTGCTGACGGGCTACCAGGTTGACAAGAACAAAGATGAC | |
| | GAGCTGACGGGTTTC | |
| 251 | ATGATCCCAGGTGGCCTGAGCGAAGCAAAACCAGCAACCCCAGAGATTCAAGA | 1018 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGCAACCGATCAGGAT | |
| | TGGCGTAAATGGGGTACGAACTACTACATCAAGGTTCGTGCGGGTGACAACAA | |
| | GTATATGCACCTGAAAGTGTTTAAGAGCCTGATCCCACTGAACTGGCAGCTGC | |
| | AGGAAGAAGATTTGGTGCTGACGGGCTACCAGGTTGACAAGAACAAAGATGAC | |
| | GAGCTGACGGGTTTC | |
| 252 | ATGATCCCAGGTGGCCTGAGCGAAGCAAAACCAGCAACCCCAGAGATTCAAGA | 1019 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCGCAGTTCGTCCAATC | |
| | TTCAAATGGGCAGGTACGAACTACTACATCAAGGTTCGTGCGGGTGACAACAA | |
| | GTATATGCACCTGAAAGTGTTTAAGAGCCTGTTCCATGGTCCACTGGATAACT | |
| | ACACCGAAGATTTGGTGCTGACGGGCTACCAGGTTGACAAGAACAAAGATGAC | |
| | GAGCTGACGGGTTTC | |
| 253 | ATGATCCCAGGTGGCCTGAGCGAAGCAAAACCAGCAACCCCAGAGATTCAAGA | 1020 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCACCCGTCCACATTTC | |
| | CCAGGTTGGAACGGTACGAACTACTACATCAAGGTTCGTGCGGGTGACAACAA | |
| | GTATATGCACCTGAAAGTGTTTAAGAGCCTGGATTTCGATCATAACTTCATCG | |
| | CAGATGAAGATTTGGTGCTGACGGGCTACCAGGTTGACAAGAACAAAGATGAC | |
| | GAGCTGACGGGTTTC | |
| 254 | ATGATCCCAGGTGGCCTGAGCGAAGCAAAACCAGCAACCCCAGAGATTCAAGA | 1021 |
| | AATTGTTGACAAAGTGAAGCCGCAGCTGGAAGAGAAAACCGGCGAAACCTACG | |
| | GCAAACTGGAAGCCGTCCAGTATAAGACTCAAGTCGTCATCTCTGATGTTTCT | |
| | GTTGGTTGGGAAGGTACGAACTACTACATCAAGGTTCGTGCGGGTGACAACAA | |
| | GTATATGCACCTGAAAGTGTTTAAGAGCCTGAGATCTGGTCCAGCACATGTTA | |
| | GAAGAGAAGATTTGGTGCTGACGGGCTACCAGGTTGACAAGAACAAAGATGAC | |
| | GAGCTGACGGGTTTC | |

Furthermore, minor modifications may also include small deletions or additions-beyond the loop 2 and loop 4 inserts described above-to the Stefin A or Stefin A derived sequences disclosed herein, such as addition or deletion of up to 10 amino acids relative to Stefin A or the Stefin A derived AFFIMER® polypeptide.

In some embodiments, the AFFIMER® agent is a PD-L1 binding AFFIMER® agent comprising an AFFIMER® polypeptide portion that binds human PD-L1 as a monomer with a dissociation constant (K_{D}) of about 1 μM or less, about 100 nM or less, about 40 nM or less, about 20 nM or less, about 10 nM or less, about 1 nM or less, or about 0.1 nM or less.

In some embodiments, the AFFIMER® agent is a PD-L1 binding AFFIMER® agent comprising an AFFIMER® polypeptide portion that binds human PD-L1 as a monomer with an off-rate constant (K_{off}), such as measured by Biacore, of about 10⁻³ s⁻¹ (e.g., unit of 1/second) or slower; of about 10⁻⁴ s⁻¹ or slower or even of about 10⁻⁵ s⁻¹ or slower.

In some embodiments, the AFFIMER® agent is a PD-L1 AFFIMER® agent comprising an AFFIMER® polypeptide portion that binds human PD-L1 as a monomer with an association constant (Kₒₙ), such as measured by Biacore, of at least about 10³ M⁻¹s⁻¹ or faster; at least about 10⁴ M⁻¹s⁻¹ or faster; at least about 10⁵ M⁻¹s⁻¹ or faster; or even at least about 10⁶ M⁻¹s⁻¹ or faster.

In some embodiments, the AFFIMER® agent is a PD-L1 AFFIMER® agent comprising an AFFIMER® polypeptide portion that binds human PD-L1 as a monomer with an IC50 in a competitive binding assay with human PD-L1 of 1 μM or less, about 100 nM or less, about 40 nM or less, about 20 nM or less, about 10 nM or less, about 1 nM or less, or about 0.1 nM or less.

In some embodiments, the AFFIMER® agent has a melting temperature (Tm, e.g., temperature at which both the folded and unfolded states are equally populated) of 65° C. or higher, and preferably at least 70° C., 75° C., 80° C. or even 85° C. or higher. Melting temperature is a particularly useful indicator of protein stability. The relative proportions of folded and unfolded proteins can be determined by many techniques known to the skilled person, including differential scanning calorimetry, UV difference spectroscopy, fluorescence, circular dichroism (CD), and NMR (Pace et al. (1997) “Measuring the conformational stability of a protein” in Protein structure: A practical approach 2:299-321).

### A. Fusions Proteins—General

In some embodiments, the AFFIMER® polypeptides may further comprise an additional insertion, substitution and/or deletion that modulates biological activity of the AFFIMER® polypeptide. For example, the additions, substitutions and/or deletions may modulate at least one property or activity of modified AFFIMER® polypeptide. For example, the additions, substitutions or deletions may modulate affinity for the AFFIMER® polypeptide, e.g., for binding to and inhibiting PD-L1, modulate the circulating half-life, modulate the therapeutic half-life, modulate the stability of the AFFIMER® polypeptide, modulate cleavage by proteases, modulate dose, modulate release or bioavailability, facilitate purification, decrease deamidation, improve shelf-life, or improve or alter a particular route of administration. Similarly, AFFIMER® polypeptides may comprise protease cleavage sequences, reactive groups, antibody-binding domains (including but not limited to, FLAG or poly-His) or other affinity-based sequences (including but not limited to, FLAG, poly-His, GST, etc.) or linked molecules (including but not limited to, biotin) that improve detection, purification or other traits of the polypeptide.

In some instances, these additional sequences are added to one end and/or the other of the AFFIMER® polypeptide in the form of a fusion protein. Accordingly, in certain aspects of the disclosure, the AFFIMER® agent is a fusion protein having at least one AFFIMER® polypeptide sequence and at least one heterologous polypeptide sequence (“fusion domain” herein). A fusion domain may be selected so as to confer a desired property, such as secretion from a cell or retention on the cell surface (e.g., for an encoded AFFIMER® construct), to serve as substrate or other recognition sequences for post-translational modifications, to create multimeric structures aggregating through protein-protein interactions, to alter (often to extend) serum half-life, or to alter tissue localization or tissue exclusion and other ADME properties-merely as examples.

For example, some fusion domains are particularly useful for isolation and/or purification of the fusion proteins, such as by affinity chromatography. Well known examples of such fusion domains that facilitate expression or purification include, merely to illustrate, affinity tags such as polyhistidine (e.g., a His₆ tag), Strep II tag, streptavidin-binding peptide (SBP) tag, calmodulin-binding peptide (CBP), glutathione S-transferase (GST), maltose-binding protein (MBP), S-tag, HA tag, c-Myc tag, thioredoxin, protein A and protein G.

In order for the AFFIMER® agent to be secreted, it will generally contain a signal sequence that directs the transport of the protein to the lumen of the endoplasmic reticulum and ultimately to be secreted (or retained on the cell surface if a transmembrane domain or other cell surface retention signal). Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence.

In some embodiments, the signal peptide is about 5 to about 40 amino acids in length (such as about 5 to about 7, about 7 to about 10, about 10 to about 15, about 15 to about 20, about 20 to about 25, or about 25 to about 30, about 30 to about 35, or about 35 to about 40 amino acids in length).

In some embodiments, the signal peptide is a native signal peptide from a human protein. In other embodiments, the signal peptide is a non-native signal peptide. For example, in some embodiments, the non-native signal peptide is a mutant native signal peptide from the corresponding native secreted human protein, and can include at least one (such as 2, 3, 4, 5, 6, 7, 8, 9, or 10 or more) substitution, insertions and/or deletions.

In some embodiments, the signal peptide is a signal peptide or mutant thereof from a non-IgSF protein family, such as a signal peptide from an immunoglobulin (such as IgG heavy chain or IgG-kappa light chain), a cytokine (such as interleukin-2 (IL-2)), a serum albumin protein (e.g. HSA or albumin), a human azurocidin preprotein signal sequence, a luciferase, a trypsinogen (e.g. chymotrypsinogen or trypsinogen) or other signal peptide able to efficiently secrete a protein from a cell. Exemplary signal peptides include but are not limited to:

TABLE 5
Exemplary Signal Sequences

| Native Protein | Signal Sequence | SEQ ID NO: |
|---|---|---|
| Human Serum Albumin | MKWVTFISLLFLFSSAYS | 1022 |
| (HSA) | | |
| Ig kappa light chain | MDMRAPAGIFGFLLVLFPGYRS | 1023 |
| Human azurocidin | MTRLTVLALLAGLLASSRA | 1024 |
| preprotein | | |
| IgG heavy chain | MELGLSWIFLLAILKGVQC | 1025 |
| IgG heavy chain | MELGLRWVFLVAILEGVQC | 1026 |
| IgG heavy chain | MKHLWFFLLLVAAPRWVLS | 1027 |
| IgG heavy chain | MDWTWRILFLVAAATGAHS | 1028 |
| IgG heavy chain | MDWTWRFLFVVAAATGVQS | 1029 |
| IgG heavy chain | MEFGLSWLFLVAILKGVQC | 1030 |
| IgG heavy chain | MEFGLSWVELVALERGVQC | 1031 |
| IgG heavy chain | MDLLHKNMKHLWFFLLLVAAPRWVLS | 1032 |
| IgG Kappa light | MDMRVPAQLLGLLLLWLSGARC | 1033 |
| IgG Kappa light | MKYLLPTAAAGLLLLAAQPAMA | 1034 |
| Gaussia luciferase | MGVKVLFALICIAVAEA | 1035 |
| Human | MAFLWLLSCWALLGTTFG | 1036 |
| chymotrypsinogen | | |
| sHuman interleukin-2 | MQLLSCIALILALV | 1037 |
| Human trypsinogen-2 | MNLLLILTFVAAAVA | 1038 |
| Human CD33 | MPLLLLLPLLWAGALA | 1039 |
| Prolactin | MDSKGSSQKGSRLLLLLVVSNLLLCQGVVS | 1040 |
| Human tPA | MDAMKRGLCCVLLLCGAVFVSPS | 1041 |
| Synthetic/Consensus | MLLLLLLLLLLALALA | 1042 |
| Synthetic/Consensus | MWWRLWWLLLLLLLLWPMVWA | 1043 |

In some embodiments of a secreted AFFIMER® agent, the recombinant polypeptide comprises a signal peptide when expressed, and the signal peptide (or a portion thereof) is cleaved from the AFFIMER® agent upon secretion.

The subject fusion proteins may also include at least one linker separating heterologous protein sequences or domains. As used herein, the term “linker” refers to a linker amino acid sequence inserted between a first polypeptide (e.g., an AFFIMER® polypeptide) and a second polypeptide (e.g., a second AFFIMER® polypeptide, an Fc region, a receptor trap, albumin, etc.). Empirical linkers designed by researchers are generally classified into 3 categories according to their structures: flexible linkers, rigid linkers, and in vivo cleavable linkers. Besides the basic role in linking the functional domains together (as in flexible and rigid linkers) or releasing free functional domain in vivo (as in in vivo cleavable linkers), linkers may offer many other advantages for the production of fusion proteins, such as improving biological activity, increasing expression yield, and achieving desirable pharmacokinetic profiles. Linkers should not adversely affect the expression, secretion, or bioactivity of the fusion protein. Linkers should not be antigenic and should not elicit an immune response.

Suitable linkers may include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. In some embodiments, the linker may comprise a cleavage site. In some embodiments, the linker may comprise an enzyme cleavage site, so that the second polypeptide may be separated from the first polypeptide.

In some embodiments, the linker can be characterized as flexible. Flexible linkers are usually applied when the joined domains require a certain degree of movement or interaction. They are generally composed of small, non-polar (e.g., Gly) or polar (e.g., Ser or Thr) amino acids. See, for example, Argos P. (1990) “An investigation of oligopeptides linking domains in protein tertiary structures and possible candidates for general gene fusion” J Mol Biol. 211:943-958. The small size of these amino acids provides flexibility and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. The most commonly used flexible linkers have sequences consisting primarily of stretches of Gly and Ser residues (“GS” linker). An example of the most widely used flexible linker has the sequence of (Gly-Gly-Gly-Gly-Ser)n (SEQ ID NO: 1044). By adjusting the copy number “n”, the length of this GS linker can be optimized to achieve appropriate separation of the functional domains, or to maintain necessary inter-domain interactions. Besides the GS linkers, many other flexible linkers have been designed for recombinant fusion proteins. As These flexible linkers are also rich in small or polar amino acids such as Gly and Ser but can contain additional amino acids such as Thr and Ala to maintain flexibility, as well as polar amino acids such as Lys and Glu to improve solubility.

In some embodiments, the linker can be characterized as rigid. While flexible linkers have the advantage to connect the functional domains passively and permitting certain degree of movements, the lack of rigidity of these linkers can be a limitation in certain fusion protein embodiments, such as in expression yield or biological activity. The ineffectiveness of flexible linkers in these instances was attributed to an inefficient separation of the protein domains or insufficient reduction of their interference with each other. Under these situations, rigid linkers have been successfully applied to keep a fixed distance between the domains and to maintain their independent functions.

Many natural linkers exhibited α-helical structures. The α-helical structure was rigid and stable, with intra-segment hydrogen bonds and a closely packed backbone. Therefore, the stiff α-helical linkers can act as rigid spacers between protein domains. George et al. (2002) “An analysis of protein domain linkers: their classification and role in protein folding” Protein Eng. 15 (11): 871-9. In general, rigid linkers exhibit relatively stiff structures by adopting α-helical structures or by containing multiple Pro residues. Under many circumstances, they separate the functional domains more efficiently than the flexible linkers. The length of the linkers can be easily adjusted by changing the copy number to achieve an optimal distance between domains. As a result, rigid linkers are chosen when the spatial separation of the domains is critical to preserve the stability or bioactivity of the fusion proteins. In this regard, alpha helix-forming linkers with the sequence of A(EAAAK)n (SEQ ID NO: 1055) have been applied to the construction of many recombinant fusion proteins. Another type of rigid linkers has a Pro-rich sequence, (XP)ₙ, with X designating any amino acid, preferably Ala, Lys, or Glu.

Merely to illustrate, exemplary linkers include:

TABLE 6
Exemplary Linkers

| Type | Sequence | SEQ ID NO: |
|---|---|---|
| Flexible | (GGGGS)ₙ (e.g., n = 1-6) | 1044 |
| Flexible | (Gly)₈ | 1045 |
| Flexible | (Gly)₆ | 1046 |
| Flexible | KESGSVSSEQLAQFRSLD | 1047 |
| Flexible | EGKSSGSGSESKST | 1048 |
| Flexible | GSAGSAAGSGEF | 1049 |
| Rigid | (EAAAK)ₙ (e.g., n = 1-6) | 1050 |
| Rigid | A(EAAAK)₄ALEA(EAAAK)₄A | 1051 |
| Rigid | PAPAP | 1052 |
| Rigid | AEAAAKEAAAKA | 1053 |
| Rigid | (Ala-Pro)n (10 to 34 aa) | 1054 |

Other linkers that may be used in the subject fusion proteins include but are not limited to, SerGly, GGSG (SEQ ID NO: 1056), GSGS (SEQ ID NO: 1057), GGGS (SEQ ID NO: 1058), S(GGS)n (SEQ ID NO: 1059) where n is 1-7, GRA, poly(Gly), poly(Ala), GGGSGGG (SEQ ID NO: 1060), ESGGGGVT (SEQ ID NO: 1061), LESGGGGVT (SEQ ID NO: 1062), GRAQVT (SEQ ID NO: 1063), WRAQVT (SEQ ID NO: 1064), and ARGRAQVT (SEQ ID NO: 1065). The hinge regions of the Fc fusions described below may also be considered linkers.

Various elements can be employed to anchor proteins on the plasma membrane of cells. For example, the transmembrane domains (TM) of type-I (oriented with the N-terminus outside the cell) and type-II (oriented with the N-terminus in the cytosol) integral membrane proteins can be used to target chimeric proteins to the plasma membrane. Proteins can also be attached to the cell surface by fusion of a GPI (glycophosphatidylinositol lipid) signal to the 3′ end of genes. Cleavage of the short carboxy-terminal peptide allows attachment of a glycolipid to the newly exposed C-terminus through an amide linkage. See Udenfriend et al. (1995) “How Glycosylphoshpatidylinositol Anchored Membrane Proteins are Made” Annu Rev Biochem 64:563-591.

In some embodiments, the fusion protein includes a transmembrane polypeptide sequence (a transmembrane domain). The distinguishing features of appropriate transmembrane polypeptides comprise the ability to be expressed at the surface of the cell on which the AFFIMER® agent is to be displayed. In some embodiments, that may be an immune cell, in particular lymphocyte cells or Natural killer (NK) cells, and once there to interact with PD-L1 so as to directing cellular response of the immune cell against a predefined target tumor cell on which PD-L1 is upregulated. The transmembrane domain can be derived either from a natural or from a synthetic source. The transmembrane domain can be derived from any membrane-bound or transmembrane protein. As non-limiting examples, the transmembrane polypeptide can be a subunit of the T cell receptor such as α, β, γ or δ, polypeptide constituting CD3 complex, IL2 receptor p55 (α chain), p75 (β chain) or γ chain, subunit chain of Fc receptors, in particular Fey receptor III or CD proteins. Alternatively, the transmembrane domain can be synthetic and can comprise predominantly hydrophobic residues such as leucine and valine.

In Some® polypeptide, a sequence that signals for the posttranslational addition of a glycosylphosphatidylinositol (GPI) anchor. GPI anchors are glycolipid structures that are added post-translationally to the C-terminus of many eukaryotic proteins. This modification to the AFFIMER® agent will cause it to be anchored (attached) on the extracellular surface of the cell membrane of the cell in which the AFFIMER® agent is re-expressed as a recombinant protein (e.g., an encoded AFFIMER® construct as described below). In these embodiments, the GPI anchor domain is C-terminal to the AFFIMER® polypeptide sequence, and preferably occurs at the C-terminus of the fusion protein.

In some embodiments, the GPI anchor domain is a polypeptide that signals for the posttranslational addition of a GPI anchor when the fusion protein of which it is a part is expressed in a eukaryotic system. The GPI anchor signal sequence consists of a set of small amino acids at the site of anchor addition (the ω site) followed by a hydrophilic spacer and ending in a hydrophobic stretch (Low, (1989) FASEB J. 3:1600-1608). Cleavage of this signal sequence occurs in the ER before the addition of an anchor with conserved central components but with variable peripheral moieties (Homans et al., Nature, 333:269-272 (1988)). The C-terminus of a GPI-anchored protein is linked through a phosphoethanolamine bridge to the highly conserved core glycan, mannose (α1-2)mannose(α1-6)mannose(α1-4)glucosamine(α1-6)myo-inositol. A phospholipid tail attaches the GPI anchor to the cell membrane.

Exemplary GPI anchor domains that can be used in the subject AFFIMER® polypeptide-containing fusion proteins include:

(SEQ ID NO: 1066)
SGTTSGTTRLLSGHTCFTLTGLLGTLVTMGLLT
(SEQ ID NO: 1067)
SGTSPGLSAGATVGIMIGVLVGVALI
(SEQ ID NO: 1068)
SAPVLSAVATVGITIGVLARVALI
(SEQ ID NO: 1069)
SSPDLSAGTAVSIMIGVLAGMALI
(SEQ ID NO: 1070)
TLGGNSASYTFVSLLFSAVTLLLLC
(SEQ ID NO: 1071)
SGTSPGLSAGATVGIMIGVLVGVALI

GPI anchor attachment can be achieved by expression of the AFFIMER® fusion protein containing the GPI anchor domain in a eukaryotic system capable of carrying out GPI posttranslational modifications. As with the transmembrane domain fusion proteins, human cells, including lymphocytes and other cells involved in initiating or promoting an antitumor are so capable and can be engineered to express and encoded AFFIMER® construct including a GPI anchor domain in order retain the expressed AFFIMER® polypeptide containing fusion on the surface of the engineered cell.

Still other modifications that can be made to the AFFIMER® polypeptide sequence or to a flanking polypeptide moiety provided as part of a fusion protein is at least one sequence that is a site for post-translational modification by an enzyme. These can include, but are not limited to, glycosylation, acetylation, acylation, lipid-modification, palmitoylation, palmitate addition, phosphorylation, glycolipid-linkage modification, and the like.

### B. Multispecific Fusion Proteins

In some embodiments, an AFFIMER® agent is a multispecific polypeptide including, for example, a first PD-L1 AFFIMER® polypeptide and at least one additional binding domain. The additional binding domain may be a polypeptide sequence selected from amongst, to illustrate, a second AFFIMER® polypeptide (which may be the same or different than the first AFFIMER® polypeptide), an antibody or fragment thereof or other antigen binding polypeptide, a ligand binding portion of a receptor (such as a receptor trap polypeptide), a receptor-binding ligand (such as a cytokine, growth factor or the like), engineered T-cell receptor, an enzyme or catalytic fragment thereof.

In some embodiments, an AFFIMER® agent includes at least one additional AFFIMER® polypeptide sequence that is also directed to PD-L1. The additional PD-L1 AFFIMER® polypeptide(s) may be the same or different (or a mixture thereof) as the first PD-L1 AFFIMER® polypeptide in order to create a multispecific AFFIMER® fusion protein. The AFFIMER® agents can bind the same or overlapping sites on PD-L1 or can bind two different sites such that the PD-L1 AFFIMER® agent can simultaneously bind two sites on the same PD-L1 protein (biparatopic) or more than two sites (multiparatopic).

In some embodiments, an AFFIMER® agent includes at least one antigen binding site from an antibody. The resulting AFFIMER® agent can be a single chain including both the PD-L1 AFFIMER® polypeptide and the antigen binding site (such as in the case of an scFv) or can be a multimeric protein complex such as in antibody assembled with heavy and/or light chains to which the sequence of the anti-PD-L1 antibody has also been fused.

In some embodiments, with respect to a multispecific AFFIMER® agent comprising a full-length immunoglobulin, the fusion of the AFFIMER® polypeptide sequence to the antibody will preserve the Fc function of the Fc region of the immunoglobulin. For example, the AFFIMER® agent may be capable of binding, via its Fc portion, to the Fc receptor of Fc receptor-positive cells. In some further embodiments, the AFFIMER® agent may activate the Fc receptor-positive cell by binding to the Fc receptor-positive cell, thereby initiating or increasing the expression of cytokines and/or co-stimulatory antigens. Furthermore, the AFFIMER® agent may transfer at least a second activation signal required for physiological activation of the T cell to the T cell via the co-stimulatory antigens and/or cytokines.

In some embodiments, resulted from the binding of its Fc portion to other cells that express Fc receptors present on the surface of effector cells from the immune system, such as immune cells, hepatocytes, and endothelial cells, the AFFIMER® agent may possess antibody-dependent cellular cytotoxicity (ADCC) function, a mechanism of cell-mediated immune defense whereby an effector cell of the immune system actively lyses a target cell, whose membrane-surface antigen has been bound by an antibody, and therefore, trigger tumor cell death via ADCC. In some further embodiments, the AFFIMER® agent is capable of demonstrating ADCC function.

As described above, apart from the Fc-mediated cytotoxicity, the Fc portion may contribute to maintaining the serum levels of the AFFIMER® agent, critical for its stability and persistence in the body. For example, when the Fc portion binds to Fc receptors on endothelial cells and on phagocytes, the AFFIMER® agent may become internalized and recycled back to the blood stream, enhancing its half-life within the body.

Exemplary targets of the additional AFFIMER® polypeptides include but are not limited to, another immune checkpoint protein, and immune co-stimulatory receptor (particularly if the additional AFFIMER® polypeptide(s) can agonize the co-stimulatory receptor), a receptor, a cytokine, a growth factor, or a tumor-associated antigen, mere to illustrate.

Where the AFFIMER® agent is an AFFIMER® polypeptide-antibody fusion protein, the immunoglobulin portion, for example, may be an immunoglobulin is a monoclonal antibody against CD20, CD30, CD33, CD38, CD52, VEGF, VEGF receptors, EGFR or Her2/neu. A few illustrative examples for such immunoglobulins include an antibody comprised within any of the following: trastuzumab, panitumumab, cetuximab, obinutuzumab, rituximab, pertuzumab, alemtuzumab, bevacizumab, tositumomab, ibritumomab, ofatumumab, brentuximab and gemtuzumab.

In some embodiments, the PD-L1 AFFIMER® polypeptide is part of an AFFIMER® agent that includes one more binding domains that inhibit an additional immune checkpoint molecule, such as those expressed on a T-cell, including but not limited to PD-L2, CTLA-4, NKG2A, KIR, LAG-3, TIM-3, CD96, VISTA, or TIGIT.

In some embodiments, the PD-L1 AFFIMER® polypeptide is part of an AFFIMER® agent that includes one more binding domains that agonizes an immune co-stimulatory molecule, such as expressed on a T-cell, including but not limited to CD28, ICOS, CD137, OX40, GITR, CD27, CD30, HVEM, DNAM-1 or CD28H.

In some embodiments, the PD-L1 AFFIMER® polypeptide is part of an AFFIMER® agent that includes one more ligand agonists of immune co-stimulatory molecules, such as an agonist ligand for CD28, ICOS, CD137, OX40, GITR, CD27, CD30, HVEM, DNAM-1 or CD28H.

In some embodiments, the PD-L1 AFFIMER® polypeptide is part of an AFFIMER® agent that includes one more binding domains that bind to a protein upregulated in the tumor microenvironment, e.g., a tumor associated antigen, such as upregulated on tumor cells in the tumor, or macrophage, fibroblasts, T-cells or other immune cells that infiltrate the tumor.

In some embodiments, the PD-L1 AFFIMER® polypeptide is part of an AFFIMER® agent that includes one more binding domains that bind to a protein selected from the groups consisting of CEACAM-1, CEACAM-5, BTLA, LAIR1, CD160, 2B4, TGFR, B7-H3, B7-H4, CD40, CD40L, CD47, CD70, CD80, CD86, CD94, CD137, CD137L, CD226, Galectin-9, GITRL, HHLA2, ICOS, ICOSL, LIGHT, MHC class I or II, NKG2a, NKG2d, OX4OL, PVR, SIRPα, TCR, CD20, CD30, CD33, CD38, CD52, VEGF, VEGF receptors, EGFR, Her2/neu, ILT1, ILT2, ILT3, ILT4, ILT5, ILT6, ILT7, ILT8, KIR2DL1, KIR2DL2, KIR2DL3, KIR2DL4, KIR2DL5A, KIR2DL5B, KIR3DL1, KIR3DL2, KIR3DL3, NKG2A, NKG2C, NKG2E or TSLP.

In some embodiments, a multispecific a PD-L1 AFFIMER® agent may further comprise a half-life extension moiety, such as any of those described herein. For example, a PD-L1 AFFIMER® agent may comprise at least one PD-L1 AFFIMER® polypeptide linked through a peptide linker to a binding domain specific for at least one immune cell (e.g., T cell and/or NK cell) binding domain (e.g., CD38 chain or CD16) further linked to a half-life extension moiety, such as a fragment crystallizable (Fc) domain or human serum albumin (HSA). In some embodiments, the half-life extension moiety is a fragment crystallizable (Fc) domain. In some embodiments, the half-life extension moiety is a human serum albumin (HSA).

### 1. Bispecific Cell Engagers

Provided herein, in some embodiments, are PD-L1 AFFIMER® agents formatted to bind to two different antigens. Non-limiting examples of such PD-L1 AFFIMER® agent formats include chemically conjugated antibodies (e.g., a PD-L1 AFFIMER® polypeptide conjugated to anti-CD16 antibody, antibody fragment, or antibody mimetic), BiTEs®, BiKEs™, and bispecific tandem diabodies.

### a) BiTEs®

In some embodiments, a PD-L1 AFFIMER® agent comprises a PD-L1 AFFIMER® polypeptide linked to a CD3-specific antibody (e.g., an anti-CD3& antibody, antibody fragment (e.g., a single variable portion, V_{H} and V_{L}, of an antibody), or antibody mimetic). For example, a PD-L1 AFFIMER® polypeptide conjugated to a CD3-specific antibody forms a bispecific T cell-engager (BiTE®) antibody-AFFIMER® complex. Canonical BiTEs® are recombinant proteins made from two flexibly linked antibody-derived binding domains. These canonical BiTE® molecules typically include a tumor-specific antigen binding domain, a peptide linker, and a T-cell-binding domain (a binding domain specific for the CD38 chain). The present disclosure provides, in some embodiments, bispecific molecules that comprise a PD-L1 AFFIMER® polypeptide as the tumor-specific antigen-binding domain, a peptide linker, and a T-cell-binding domain (e.g., a binding domain specific for the CD38 chain). Binding of these bispecific molecules to CD38 chain promotes T cell-mediated anti-tumor activity after engaging with PD-L1 antigen, directing the activity of CD3 T cells, such as CD3 CD8 T cells, towards PD-L1 cells. This allows circulating T cells in a subject to be redirected towards PD-L1 cells (e.g., tumor cells) without the need for ex vivo expression of a CAR. See, e.g., Aigner M. et al. Leukemia 2013; 27:1107-1115 for a description of a PD-L1/CD3-bispecific BiTE® antibody construct.

### b) BiKEs™

In some embodiments, a PD-L1 AFFIMER® agent comprises a PD-L1 AFFIMER® polypeptide linked to a CD16-specific antibody (e.g., an anti-CD16 antibody, antibody fragment (e.g., a single variable portion, V_{H} and V_{L}, of an antibody), or antibody mimetic). For example, a PD-L1 AFFIMER® polypeptide conjugated to a CD16-specific antibody forms a bispecific NK cell-engager (BiKE™) antibody-AFFIMER® complex. Canonical BiTEs comprise two antibody fragments, a first recognizing a tumor antigen and a second directed against CD16 on NK cells, which together trigger antibody-dependent cell-mediated cytotoxicity.

In some embodiments, a PD-L1 AFFIMER® agent comprises a PD-L1 AFFIMER® polypeptide linked to a single-chain variable fragment (scFv) domain specific for CD16 on NK cells.

### c) Bispecific Tandem Binders

In some embodiments, a PD-L1 AFFIMER® agent is formatted as a bispecific tetravalent molecule. For example, a PD-L1 AFFIMER® agent may be formatted as a single chain construct constructed by linking two PD-L1 AFFIMER® polypeptides to two antibody variable domains (VH and VL) with specificities for human CD3 (T cell antigen).

### 2. Trispecific Cell Engagers

Also provided herein, in some embodiments, are PD-L1 AFFIMER® agents formatted to bind to associate with three different antigens. Non-limiting examples of such PD-L1 AFFIMER® agent formats include TriKEs™, TriNKETs™ and tandem triple scFvs.

### a) TriKEs™

In some embodiments, a PD-L1 AFFIMER® agent comprises a PD-L1 AFFIMER® polypeptide linked to human interleukin (IL)-15 and a CD16-specific antibody (e.g., an anti-CD16 antibody, antibody fragment (e.g., a single variable portion, V_{H} and V_{L}, of an antibody), or antibody mimetic). For example, a PD-L1 AFFIMER® agent may comprise a single chain variable fragments (scFv) that is crosslinked with human IL-15 and a PD-L1 AFFIMER® polypeptide that is crosslinked with the human IL-15 to form a trispecific NK cell-engager (TriKE™) antibody-AFFIMER® complex. The scFv recognizes the anti-CD16 marker on NK cells, and the PD-L1 AFFIMER® polypeptide recognizes PD-L1 expressed on the tumor cell. The IL-15 component of TriKE provides a self-sustaining signal that activates NK cells and enhances their ability to kill tumor cells. Compared to BikEs™, TriKEs™ elicit superior NK cytotoxicity and NK cell persistence in a xenograft tumor model in vivo and are proposed to be effective adjuncts to existing NK transfer protocols.

In some embodiments, a PD-L1 AFFIMER® agent comprises a PD-L1 AFFIMER® polypeptide and a single-chain variable fragment (scFv) domain specific for CD16 on natural killer (NK) cells, each crosslinked to an IL-15 protein. This PD-L1 AFFIMER® agent is capable of, for example, directing NK cells to tumors by facilitating formation of intracellular synapses, binding CD16 on NK cells to trigger ADC, and driving in vivo NK cell expansion. IL-15 promotes NK cell activation, expansion and survival.

For a review of BiKE™ and TriKE™ technology, see Felices M et al. Methods Mol Bio. 2016; 1441:333-346, incorporated herein by reference.

### b) TriNKETS™

Tri-specific, NK cell Engager Therapies (TriNKETs™) are also encompassed by the present disclosure. In some embodiments, a PD-L1 AFFIMER® agent comprises a PD-L1 AFFIMER® polypeptide linked to a domain that binds an NKG2D receptor on NK cells and a domain that binds a CD16 receptor on natural killer cells. Such PD-L1 AFFIMER® agents can engage more than one kind of NK activating receptor and may block the binding of natural ligands to NKG2D. In some embodiments, these PD-L1 AFFIMER® agents can agonize NK cells in humans. See International Publication No. WO2019/164930, incorporated herein by reference.

In some embodiments, a PD-L1 AFFIMER® agent comprises (a) an antibody (e.g., an antibody fragment or antibody mimetic) that binds NKG2D; (b) a PD-L1 AFFIMER® polypeptide; and (c) an antibody (e.g., an antibody fragment or antibody mimetic) that binds CD16.

In some embodiments, a PD-L1 AFFIMER® agent comprises (a) an antibody Fab fragment that binds NKG2D; (b) a PD-L1 AFFIMER® polypeptide; and (c) an antibody Fc domain that binds CD16. In some embodiments, the PD-L1 AFFIMER® polypeptide is linked to the antibody Fab fragment or the antibody Fc domain via a hinge comprising Ala-Ser or Gly-Ala-Ser.

### c) Tandem Triple Binders

In some embodiments, a PD-L1 AFFIMER® agent is formatted as a tandem triple scFv molecule. For example, a PD-L1 AFFIMER® agent may comprise two PD-L1 AFFIMER® polypeptides linked to an scFv domain specific for CD16. In some embodiments, a PD-L1 AFFIMER® agent may comprise a PD-L1 AFFIMER® polypeptide linked to an scFv domain specific for CD16 and an scFv specific for CD123.

### 3. Tetraspecific Cell Engagers

Also provided herein, in some embodiments, are PD-L1 AFFIMER® agents formatted to associates with four different antigens. A non-limiting examples of such a PD-L1 AFFIMER® agent format includes TetraKEs™. In some embodiments, a PD-L1 AFFIMER® agent comprises a PD-L1 AFFIMER® polypeptide and a single-chain variable fragment (scFv) domain specific for CD16 on natural killer (NK) cells, each crosslinked to an IL-15 protein, and further comprising an scFv that specifically binds to CD133 on cancer stem cells in order to promote ADCC. In some embodiments, the scFv that specifically binds to CD133 is linked to the PD-L1 AFFIMER® polypeptide through a hinge region (e.g., mutated IgG/hinge). See, e.g., Schmohl J U et al. Oncotarget. 2016; 7 (45): 73830.

### 4. Engineered Cells

PD-L1 AFFIMER® polypeptides, in some embodiments, are linked to an immune cell. Adoptive cell therapy, also known as cellular immunotherapy, is a form of treatment that uses immune to treat cancer. Non-limiting examples of adoptive cell therapies include tumor-infiltrating lymphocyte (TIL) therapy, engineered T cell receptor (TCR) therapy, chimeric antigen receptor (CAR) T cell therapy, and natural killer (NK) cell therapy.

TIL therapy involves naturally occurring T cells that have already infiltrated patients' tumors, and then activating and expands the. Then, large numbers of these activated T cells are re-infused into patients, where they can then seek out and destroy tumors. In some embodiments, TILs are linked to or modified to express a PD-L1 AFFIMER® polypeptides.

TCR therapy involves taking T cells from patients, but instead of just activating and expanding the available anti-tumor T cells, the T cells can also be equipped with a new T cell receptor that enables them to target specific cancer antigens. In some embodiments, T cells (e.g., CD8+ T cells) obtained from a patient are equipped with a PD-L1 AFFIMER® polypeptide.

Canonical TIL and TCR therapies can only target and eliminate cancer cells that present their antigens in a certain context (e.g., when the antigens are bound by the major histocompatibility complex, or MHC). To overcome this limitation, a patient's T cells can be equipped with a synthetic receptor known as a CAR. A CAR comprises an antigen-binding fragment, at least one hinge and/or linker domain, a transmembrane domain, and at least one intracellular signaling domain. Binding of the antigen-binding fragment to a target antigen, such as PD-L1, results in transduction of a signal mediated by the intracellular signaling domains of the CAR. CARs are typically expressed on the surface of a T cell, such as a CD8+ T cell, to direct the activity of the T cell towards cells expressing the antigen recognized by the CAR. CD8+ T cells expressing CARs with antigen-binding fragments specific to PD-L1 are useful for targeted killing of PD-L1+ cells, such as cancer cells. See, e.g., Dutour A et al., Advances in Hematology 2012 Article ID 683065.

More recently, adoptive cell therapy strategies have begun to incorporate other immune cells, such as Natural Killer (NK) cells. One application being explored in the clinic involves equipping these NK cells with cancer-targeting CARs. In some embodiments, NK cells are linked to or modified to express an PD-L1 AFFIMER® polypeptide.

In some embodiments, a PD-L1 AFFIMER® agent comprises a PD-L1 AFFIMER® polypeptide, a linker sequence, a transmembrane domain, and a CD3z signaling domain. In other embodiments, a PD-L1 AFFIMER® agent comprises a PD-L1 AFFIMER® polypeptide, a linker sequence, a transmembrane domain, a CD28 co-stimulatory domain, and a CD3z signaling domain. In yet other embodiments, a PD-L1 AFFIMER® agent comprises a PD-L1 AFFIMER® polypeptide, a linker sequence, a transmembrane domain, a 4-1BB co-stimulatory domain, and a CD3z signaling domain. In still other embodiments, a PD-L1 AFFIMER® agent comprises a PD-L1 AFFIMER® polypeptide, a linker sequence, a transmembrane domain, a CD28 co-stimulatory domain and a 4-1BB co-stimulatory domain, and a CD3z signaling domain.

Also provided herein are immune cells, e.g., T cells or NK cells, comprising any one or more of the PD-L1 AFFIMER® agent described above.

### C. Engineering PK and ADME Properties

In some embodiment, the AFFIMER® agent may not have a half-life and/or PK profile that is optimal for the route of administration, such as parenteral therapeutic dosing. A “half-life” is the amount of time it takes for a substance, such as an AFFIMER® agent of the present disclosure, to lose half of its pharmacologic or physiologic activity or concentration. Biological half-life can be affected by elimination, excretion, degradation (e.g., enzymatic) of the substance, or absorption and concentration in certain organs or tissues of the body. In some embodiments, biological half-life can be assessed by determining the time it takes for the blood plasma concentration of the substance to reach half its steady state level (“plasma half-life”). To address this shortcoming, there are a variety of general strategies for prolongation of half-life that have been used in the case of other protein therapeutics, including the incorporation of half-life extending moieties as part of the AFFIMER® agent.

The term “half-life extending moiety” refers to a pharmaceutically acceptable moiety, domain, or molecule covalently linked (chemically conjugated or fused) to an AFFIMER® polypeptide to form an AFFIMER® agent described herein, optionally via a non-naturally encoded amino acid, directly or via a linker, that prevents or mitigates in vivo proteolytic degradation or other activity-diminishing modification of the AFFIMER® polypeptide, increases half-life, and/or improves or alters other pharmacokinetic or biophysical properties including but not limited to increasing the rate of absorption, reducing toxicity, improving solubility, reducing protein aggregation, increasing biological activity and/or target selectivity of the modified AFFIMER® polypeptide, increasing manufacturability, and/or reducing immunogenicity of the modified AFFIMER® polypeptide, compared to a comparator such as an unconjugated form of the modified AFFIMER® polypeptide. The term “half-life extending moiety” includes non-proteinaceous, half-life extending moieties, such as a water soluble polymer such as polyethylene glycol (PEG) or discrete PEG, hydroxyethyl starch (HES), a lipid, a branched or unbranched acyl group, a branched or unbranched C8-C30 acyl group, a branched or unbranched alkyl group, and a branched or unbranched C8-C30 alkyl group; and proteinaceous half-life extending moieties, such as serum albumin, transferrin, adnectins (e.g., albumin-binding or pharmacokinetics extending (PKE) adnectins), Fc domain, and unstructured polypeptide, such as XTEN and PAS polypeptide (e.g. conformationally disordered polypeptide sequences composed of the amino acids Pro, Ala, and/or Ser), and a fragment of any of the foregoing. An examination of the crystal structure of an AFFIMER® polypeptide and its interaction with its target, can indicate which certain amino acid residues have side chains that are fully or partially accessible to solvent.

In some embodiments, the half-life extending moiety extends the half-life of the resulting AFFIMER® agent circulating in mammalian blood serum compared to the half-life of the protein that is not so conjugated to the moiety (such as relative to the AFFIMER® polypeptide alone). In some embodiments, half-life is extended by greater than or greater than about 1.2-fold, 1.5-fold, 2.0-fold, 3.0-fold, 4.0-fold, 5.0-fold, or 6.0-fold. In some embodiments, half-life is extended by more than 6 hours, more than 12 hours, more than 24 hours, more than 48 hours, more than 72 hours, more than 96 hours or more than 1 week after in vivo administration compared to the protein without the half-life extending moiety.

As means for further exemplification, half-life extending moieties that can be used in the generation of AFFIMER® agents of the disclosure include:
Genetic fusion of the pharmacologically AFFIMER® sequence to a naturally long-half-life protein or protein domain (e.g., Fc fusion, transferrin [Tf] fusion, or albumin fusion. See, for example, Beck et al. (2011) “Therapeutic Fc-fusion proteins and peptides as successful alternatives to antibodies. MAbs. 3:1-2; Czajkowsky et al. (2012) “Fc-fusion proteins: new developments and future perspectives. EMBO Mol Med. 4:1015-28; Huang et al. (2009) “Receptor-Fc fusion therapeutics, traps, and Mimetibody technology” Curr Opin Biotechnol. 2009; 20:692-9; Keefe et al. (2013) “Transferrin fusion protein therapies: acetylcholine receptor-transferrin fusion protein as a model. In: Schmidt S, editor. Fusion protein technologies for biopharmaceuticals: applications and challenges. Hoboken: Wiley; p. 345-56; Weimer et al. (2013) “Recombinant albumin fusion proteins. In: Schmidt S, editor. Fusion protein technologies for biopharmaceuticals: applications and challenges. Hoboken: Wiley; 2013. p. 297-323; Walker et al. (2013) “Albumin-binding fusion proteins in the development of novel long-acting therapeutics. In: Schmidt S, editor. Fusion protein technologies for biopharmaceuticals: applications and challenges. Hoboken: Wiley; 2013. p. 325-43.
Genetic fusion of the pharmacologically AFFIMER® sequence to an inert polypeptide, e.g., XTEN (also known as recombinant PEG or “rPEG”), a homoamino acid polymer (HAP; HAPylation), a proline-alanine-serine polymer (PAS; PASylation), or an elastin-like peptide (ELP; ELPylation). See, for example, Schellenberger et al. (2009) “A recombinant polypeptide extends the in vivo half-life of peptides and proteins in a tunable manner. Nat Biotechnol. 2009; 27:1186-90; Schlapschy et al. Fusion of a recombinant antibody fragment with a homo-amino-acid polymer: effects on biophysical properties and prolonged plasma half-life. Protein Eng Des Sel. 2007; 20:273-84; Schlapschy (2013) PASylation: a biological alternative to PEGylation for extending the plasma halflife of pharmaceutically active proteins. Protein Eng Des Sel. 26:489-501. Floss et al. (2012) “Elastin-like polypeptides revolutionize recombinant protein expression and their biomedical application. Trends Biotechnol. 28:37-45. Floss et al. “ELP-fusion technology for biopharmaceuticals. In: Schmidt S, editor. Fusion protein technologies for biopharmaceuticals: application and challenges. Hoboken: Wiley; 2013. p. 372-98.
Increasing the hydrodynamic radius by chemical conjugation of the pharmacologically active peptide or protein to repeat chemical moieties, e.g., to PEG (PEGylation) or hyaluronic acid. See, for example, Caliceti et al. (2003) “Pharmacokinetic and biodistribution properties of poly(ethylene glycol)-protein conjugates” Adv Drug Delivery Rev. 55:1261-77; Jevsevar et al. (2010) PEGylation of therapeutic proteins. Biotechnol J 5:113-28; Kontermann (2009) “Strategies to extend plasma half-lives of recombinant antibodies” BioDrugs. 23:93-109; Kang et al. (2009) “Emerging PEGylated drugs” Expert Opin Emerg Drugs. 14:363-80; and Mero et al. (2013) “Conjugation of hyaluronan to proteins” Carb Polymers. 92:2163-70.
Significantly increasing the negative charge of fusing the pharmacologically active peptide or protein by polysialylation; or, alternatively, (b) fusing a negatively charged, highly sialylated peptide (e.g., carboxy-terminal peptide [CTP; of chorionic gonadotropin (CG) b-chain]), known to extend the half-life of natural proteins such as human CG b-subunit, to the biological drug candidate. See, for example, Gregoriadis et al. (2005) “Improving the therapeutic efficacy of peptides and proteins: a role for polysialic acids” Int J Pharm. 2005; 300:125-30; Duijkers et al. “Single dose pharmacokinetics and effects on follicular growth and serum hormones of a long-acting recombinant FSH preparation (FSHCTP) in healthy pituitary-suppressed females” (2002) Hum Reprod. 17:1987-93; and Fares et al. “Design of a longacting follitropin agonist by fusing the C-terminal sequence of the chorionic gonadotropin beta subunit to the follitropin beta subunit” (1992) Proc Natl Acad Sci USA. 89:4304-8. 35; and Fares “Half-life extension through O-glycosylation.
Binding non-covalently, via attachment of a peptide or protein-binding domain to the bioactive protein, to normally long-half-life proteins such as HSA, human IgG, transferrin or fibronectin. See, for example, Andersen et al. (2011) “Extending half-life by indirect targeting of the neonatal Fc receptor (FcRn) using a minimal albumin binding domain” J Biol Chem. 286:5234-41; O'Connor-Semmes et al. (2014) “GSK2374697, a novel albumin-binding domain antibody (albudAb), extends systemic exposure of extendin-4: first study in humans-PK/PD and safety” Clin Pharmacol Ther. 2014; 96:704-12. Sockolosky et al. (2014) “Fusion of a short peptide that binds immunoglobulin G to a recombinant protein substantially increases its plasma half-life in mice” PLOS One. 2014; 9: e102566.

Classical genetic fusions to long-lived serum proteins offer an alternative method of half-life extension distinct from chemical conjugation to PEG or lipids. Two major proteins have traditionally been used as fusion partners: antibody Fc domains and human serum albumin (HSA). Fc fusions involve the fusion of peptides, proteins or receptor exodomains to the Fc portion of an antibody. Both Fc and albumin fusions achieve extended half-lives not only by increasing the size of the peptide drug, but both also take advantage of the body's natural recycling mechanism: the neonatal Fc receptor, FcRn. The pH-dependent binding of these proteins to FcRn prevents degradation of the fusion protein in the endosome. Fusions based on these proteins can have half-lives in the range of 3-16 days, much longer than typical PEGylated or lipidated peptides. Fusion to antibody Fc domains can improve the solubility and stability of the peptide or protein drug. An example of a peptide Fc fusion is dulaglutide, a GLP-1 receptor agonist currently in late-stage clinical trials. Human serum albumin, the same protein exploited by the fatty acylated peptides is the other popular fusion partner. Albiglutide is a GLP-1 receptor agonist based on this platform. A major difference between Fc and albumin is the dimeric nature of Fc versus the monomeric structure of HSA leading to presentation of a fused peptide as a dimer or a monomer depending on the choice of fusion partner. The dimeric nature of an AFFIMER® polypeptide-Fc fusion can produce an avidity effect if the AFFIMER® polypeptide target, such as CD33 on tumor cells, are spaced closely enough together or are themselves dimers. This may be desirable or not depending on the target.

### 1. Fc Fusions

In some embodiments, the AFFIMER® polypeptide may be part of a fusion protein with an immunoglobulin Fc domain (“Fc domain”), or a fragment or variant thereof, such as a functional Fc region. In this context, an Fc fusion (“Fc-fusion”), such as a PD-L1 AFFIMER® agent created as an AFFIMER® polypeptide-Fc fusion protein, is a polypeptide comprising at least one PD-L1AFFIMER® polypeptide sequence covalently linked through a peptide backbone (directly or indirectly) to an Fc region of an immunoglobulin. An Fc-fusion may comprise, for example, the Fc region of an antibody (which facilitates effector functions and pharmacokinetics) and a PD-L1 AFFIMER® polypeptide sequence as part of the same polypeptide. An immunoglobulin Fc region may also be linked indirectly to at least one PD-L1 AFFIMER® polypeptide. Various linkers are known in the art and can optionally be used to link an Fc to a polypeptide including a PD-L1 AFFIMER® polypeptide sequence to generate an Fc-fusion. In some embodiments, Fc-fusions can be dimerized to form Fc-fusion homodimers, or using non-identical Fc domains, to form Fc-fusion heterodimers.

In some embodiments, an Fc-fusion homodimer comprises a dimer of a PD-L1 AFFIMER® agent that comprises a PD-L1 AFFIMER® polypeptide linked to an Fc domain linked to another PD-L1 AFFIMER® polypeptide (PD-L1 AFFIMER® polypeptide-Fc domain-PD-L1 AFFIMER® polypeptide).

There are several reasons for choosing the Fc region of human antibodies for use in generating PD-L1 AFFIMER® agents as PD-L1 AFFIMER® fusion proteins. The principle rationale is to produce a stable protein, large enough to demonstrate a similar pharmacokinetic profile compared with those of antibodies, and to take advantage of the properties imparted by the Fc region; this includes the salvage neonatal FcRn receptor pathway involving FcRn-mediated recycling of the fusion protein to the cell surface post endocytosis, avoiding lysosomal degradation and resulting in release back into the bloodstream, thus contributing to an extended serum half-life. Another obvious advantage is the Fc domain's binding to Protein A, which can simplify downstream processing during production of the AFFIMER® agent and permit generation of highly pure preparation of the AFFIMER® agent.

In general, an Fc domain will include the constant region of an antibody excluding the first constant region immunoglobulin domain. Thus, Fc domain refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ2. Although the boundaries of the Fc domain may vary, the human IgG heavy chain Fc region is usually defined to comprise residues C226 or P230 to its carboxyl-terminus, wherein the numbering is according to the EU index as set forth in Kabat (Kabat et al., Sequences of Proteins of Immunological Interest, 5th Ed. Public Health Service, NIH, Bethesda, Md. (1991)). Fc may refer to this region in isolation, or this region in the context of a whole antibody, antibody fragment, or Fc fusion protein. Polymorphisms have been observed at a number of different Fc positions and are also included as Fc domains as used herein.

In some embodiments, the Fc As used herein, a “functional Fc region” refers to an Fc domain or fragment thereof which retains the ability to bind FcRn. A functional Fc region binds to FcRn but does not possess effector function. The ability of the Fc region or fragment thereof to bind to FcRn can be determined by standard binding assays known in the art. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions can be assessed using various assays known in the art for evaluating such antibody effector functions.

In an exemplary embodiment, the Fc domain is derived from an IgG1 subclass, however, other subclasses (e.g., IgG2, IgG3, and IgG4) may also be used. An exemplary sequence of a human IgG1 immunoglobulin Fc domain which can be used is:

(SEQ ID NO: 1072)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK

In some embodiments, the Fc region used in the fusion protein may comprise the hinge region of an Fc molecule. An exemplary hinge region comprises the core hinge residues spanning positions 1-16 (e.g., DKTHTCPPCPAPELLG (SEQ ID NO: 1073)) of the exemplary human IgG1 immunoglobulin Fc domain sequence provided above. In some embodiments, the AFFIMER® polypeptide-containing fusion protein may adopt a multimeric structure (e.g., dimer) owing, in part, to the cysteine residues at positions 6 and 9 within the hinge region of the exemplary human IgG1 immunoglobulin Fc domain sequence provided above. In other embodiments, the hinge region as used herein, may further include residues derived from the CH1 and CH2 regions that flank the core hinge sequence of the exemplary human IgG1 immunoglobulin Fc domain sequence provided above. In yet other embodiments, the hinge sequence may comprise or consist of GSTHTCPPCPAPELLG (SEQ ID NO: 1074) or EPKSCDKTHTCPPCPAPELLG (SEQ ID NO: 1075).

In some embodiments, the hinge sequence may include at least one substitution that confer desirable pharmacokinetic, biophysical, and/or biological properties. Some exemplary hinge sequences include:

(SEQ ID NO: 1076)
EPKSCDKTHTCPPCPAPELLGGPS;
(SEQ ID NO: 1077)
EPKSSDKTHTCPPCPAPELLGGPS;
(SEQ ID NO: 1078)
EPKSSDKTHTCPPCPAPELLGGSS;
(SEQ ID NO: 1079)
EPKSSGSTHTCPPCPAPELLGGSS;
(SEQ ID NO: 1080)
DKTHTCPPCPAPELLGGPS;
and
(SEQ ID NO: 1081)
DKTHTCPPCPAPELLGGSS.

In some embodiments, the residue P at position 18 of the exemplary human IgG1 immunoglobulin Fc domain sequence provided above may be replaced with S to ablate Fc effector function; this replacement is exemplified in hinges having the sequences EPKSSDKTHTCPPCPAPELLGGSS (SEQ ID NO: 1078), EPKSSGSTHTCPPCPAPELLGGSS (SEQ ID NO: 1079), and DKTHTCPPCPAPELLGGSS (SEQ ID NO: 1081). In another embodiment, the residues DK at positions 1-2 of the exemplary human IgG1 immunoglobulin Fc domain sequence provided above may be replaced with GS to remove a potential clip site; this replacement is exemplified in the sequence EPKSSGSTHTCPPCPAPELLGGSS (SEQ ID NO: 1079). In another embodiment, the C at the position 103 of the heavy chain constant region of human IgG1 (e.g., domains CH1-CH3), may be replaced with S to prevent improper cysteine bond formation in the absence of a light chain; this replacement is exemplified by EPKSSDKTHTCPPCPAPELLGGPS (SEQ ID NO: 1077), EPKSSDKTHTCPPCPAPELLGGSS (SEQ ID NO: 1078), and EPKSSGSTHTCPPCPAPELLGGSS (SEQ ID NO: 1079).

In some embodiments, the Fc is a mammalian Fc such as a human Fc, including Fc domains derived from IgG1, IgG2, IgG3 or IgG4. The Fc region may possess at least about 80%, 85%, 90%, 95%, 96%, 97%, 98%, or 99% sequence identity with a native Fc region and/or with an Fc region of a parent polypeptide. In some embodiments, the Fc region may have at least about 90% sequence identity with a native Fc region and/or with an Fc region of a parent polypeptide.

In some embodiments, the Fc domain comprises an amino acid sequence selected from SEQ ID NO: 1082 to 1095 or an Fc sequence from the examples provided by SEQ ID NOs: 1082 to 1095. It should be understood that the C-terminal lysine of an Fc domain is an optional component of a fusion protein comprising an Fc domain. In some embodiments, the Fc domain comprises an amino acid sequence selected from SEQ ID NOs: 1082 to 1095, except that the C-terminal lysine thereof is omitted. In some embodiments, the Fc domain comprises the amino acid sequence selected from SEQ ID NO: 1082 to 1095. In some embodiments, the Fc domain comprises the amino acid sequence selected from SEQ ID NO: 1082 to 1095 except the C-terminal lysine thereof is omitted.

TABLE 7
Exemplary Immunoglobulin Sequences

| | | SEQ ID |
|---|---|---|
| Name | Sequence | NO: |
| hIgG1a_191 | DKTHTCPPCPAPELLGGPSVFLFPPKPKDTLMISRTPEV | 1082 |
| [A subtype] | TCVVVDVSHEDPEVKFNWYVDGVEVHNAKTKPREEQYNS | |
| | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGNVFSCSVMHEALHNHYTQKSLSLSPGK | |
| hIgG1a_189 | DKTHTCPPCPAPELLGGPSVFLFPPKPKDTLMISRTPEV | 1083 |
| [hIgG1a_191 sans “GK” | TCVVVDVSHEDPEVKFNWYVDGVEVHNAKTKPREEQYNS | |
| on C term; A subtype] | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGNVFSCSVMHEALHNHYTQKSLSLSP | |
| hIgG1a_191b | DKTHTCPPCPAPELLGGPSVFLFPPKPKDTLMISRTPEV | 1084 |
| [A/F subtype] | TCVVVDVSHEDPEVKFNWYVDGVEVHNAKTKPREEQYNS | |
| | TYRVVSVLTVLHQDWLNGKEYKCKVSNKALPAPIEKTIS | |
| | KAKGQPREPQVYTLPPSRDEMTKNQVSLTCLVKGFYPSD | |
| | IAVEWESNGQPENNYKTTPPVLDSDGSFFLYSKLTVDKS | |
| | RWQQGNVFSCSVMHEALHNHYTQKSLSLSPGK | |
| hIgG1f_1.1_191 | DKTHTCPPCPAPEAEGAPSVFLFPPKPKDTLMISRTPEV | 1085 |
| [Contains five point- | TCVVVDVSHEDPEVKFNWYVDGVEVHNAKTKPREEQYNS | |
| mutations to alter | TYRVVSVLTVLHQDWLNGKEYKCKVSNKALPSSIEKTIS | |
| ADCC function, F | KAKGQPREPQVYTLPPSREEMTKNQVSLTCLVKGFYPSD | |
| subtype] | IAVEWESNGQPENNYKTTPPVLDSDGSFFLYSKLTVDKS | |
| | RWQQGNVFSCSVMHEALHNHYTQKSLSLSPGK | |
| hIgG1f_1.1 186 | EPKSSDKTHTCPPCPAPEAEGAPSVFLFPPKPKDTLMIS | 1086 |
| [Contains five point- | RTPEVTCVVVDVSHEDPEVKFNWYVDGVEVHNAKTKPRE | |
| mutations to alter | EQYNSTYRVVSVLTVLHQDWLNGKEYKCKVSNKALPSSI | |
| ADCC function and | EKTISKAKGQPREPQVYTLPPSREEMTKNQVSLTCLVKG | |
| C225S (Edlemen | FYPSDIAVEWESNGQPENNYKTTPPVLDSDGSFFLYSKL | |
| numbering); F subtype] | TVDKSRWQQGNVFSCSVMHEALHNHYTQKSLSLSPGK | |
| hIgG1a_(N297G)_191 | DKTHTCPPCPAPELLGGPSVFLFPPKPKDTLMISRTPEV | 1087 |
| [A subtype] | TCVVVDVSHEDPEVKFNWYVDGVEVHNAKTKPREEQYGS | |
| | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGNVFSCSVMHEALHNHYTQKSLSLSPGK | |
| hIgG1a_190 | DKTHTCPPCPAPELLGGPSVFLFPPKPKDTLMISRTPEV | 1088 |
| [hIgG1a_190 sans “K” | TCVVVDVSHEDPEVKFNWYVDGVEVHNAKTKPREEQYNS | |
| on C term; A subtype] | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGNVFSCSVMHEALHNHYTQKSLSLSPG | |
| hIgG1a_(N297Q)_191 | DKTHTCPPCPAPELLGGPSVFLFPPKPKDTLMISRTPEV | 1089 |
| [A subtype] | TCVVVDVSHEDPEVKFNWYVDGVEVHNAKTKPREEQYQS | |
| | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGNVFSCSVMHEALHNHYTQKSLSLSPGK | |
| hIgG1a_(N297S)_191 | DKTHTCPPCPAPELLGGPSVFLFPPKPKDTLMISRTPEV | 1090 |
| [A subtype] | TCVVVDVSHEDPEVKFNWYVDGVEVHNAKTKPREEQYSS | |
| | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGNVFSCSVMHEALHNHYTQKSLSLSPGK | |
| hIgG1a_(N297A)_191 | DKTHTCPPCPAPELLGGPSVFLFPPKPKDTLMISRTPEV | 1091 |
| [A subtype] | TCVVVDVSHEDPEVKFNWYVDGVEVHNAKTKPREEQYAS | |
| | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGNVFSCSVMHEALHNHYTQKSLSLSPGK | |
| hIgG1a_(N297H)_191 | DKTHTCPPCPAPELLGGPSVFLFPPKPKDTLMISRTPEV | 1092 |
| [A subtype] | TCVVVDVSHEDPEVKFNWYVDGVEVHNAKTKPREEQYHS | |
| | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGNVFSCSVMHEALHNHYTQKSLSLSPGK | |
| hIgG4 | DKRVESKYGPPCPSCPAPEFLGGPSVFLFPPKPKDTLMI | 1093 |
| | SRTPEVTCVVVDVSQEDPEVQFNWYVDGVEVHNAKTKPR | |
| | EEQFNSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLGK | |
| hIgG4_(S241P) | DKRVESKYGPPCPPCPAPEFLGGPSVFLFPPKPKDTLMI | 1094 |
| | SRTPEVTCVVVDVSQEDPEVQFNWYVDGVEVHNAKTKPR | |
| | EEQFNSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLGK | |
| hIgG1 (Contain two | SEPKSCDKTHTCPPCPAPEAAGGPSVFLFPPKPKDTLMI | 1095 |
| point-mutations to alter | SRTPEVTCVVVDVSHEDPEVKFNWYVDGVEVHNAKTKPR | |
| ADCC function L20A, | EEQYNSTYRVVSVLTVLHQDWLNGKEYKCKVSNKALPAP | |
| L21A) | IEKTISKAKGQPREPQVYTLPPSREEMTKNQVSLTCLVK | |
| | GFYPSDIAVEWESNGQPENNYKTTPPVLDSDGSFFLYSK | |
| | LTVDKSRWQQGNVFSCSVMHEALHNHYTQKSLSLSPGK | |

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins.

In some embodiments, the fusion protein includes an Fc domain sequence for which the resulting AFFIMER® agent has no (or reduced) ADCC and/or complement activation or effector functionality. For example, the Fc domain may comprise a naturally disabled constant region of IgG2 or IgG4 isotype or a mutated IgG1 constant region. Examples of suitable modifications are described in EP0307434. One example comprises the substitutions of alanine residues at positions 235 and 237 (EU index numbering).

In other embodiments, the fusion protein includes an Fc domain sequence for which the resulting AFFIMER® agent will retain some or all Fc functionality for example will be capable of one or both of ADCC and CDC activity, as for example if the fusion protein comprises the Fc domain from human IgG1 or IgG3. Levels of effector function can be varied according to known techniques, for example by mutations in the CH2 domain, for example wherein the IgG1 CH2 domain has at least one mutation at positions selected from 239 and 332 and 330, for example the mutations are selected from S239D and 1332E and A330L such that the antibody has enhanced effector function, and/or for example altering the glycosylation profile of the antigen-binding protein of the disclosure such that there is a reduction in fucosylation of the Fc region.

### 2. Albumin Fusions

In some embodiments, the AFFIMER® agent is a fusion protein comprising, in addition to at least one AFFIMER® polypeptide sequence, an albumin sequence or an albumin fragment. In other embodiments, the AFFIMER® agent is conjugated to the albumin sequence or an albumin fragment through chemical linkage other than incorporation into the polypeptide sequence including the AFFIMER® polypeptide. In some embodiments, the albumin, albumin variant, or albumin fragment is human serum albumin (HSA), a human serum albumin variant, or a human serum albumin fragment. Albumin serum proteins comparable to HSA are found in, for example, cynomolgus monkeys, cows, dogs, rabbits and rats. Of the non-human species, bovine serum albumin (BSA) is the most structurally similar to HSA. See, e.g., Kosa et al., (2007) J Pharm Sci. 96 (11): 3117-24. The present disclosure contemplates the use of albumin from non-human species, including, but not limited to, albumin sequence derived from cyno serum albumin or bovine serum albumin.

Mature HSA, a 585 amino acid polypeptide (approx. 67 kDa) having a serum half-life of about 20 days, is primarily responsible for the maintenance of colloidal osmotic blood pressure, blood pH, and transport and distribution of numerous endogenous and exogenous ligands. The protein has three structurally homologous domains (domains I, II and III), is almost entirely in the alpha-helical conformation, and is highly stabilized by 17 disulfide bridges. In some embodiments, the AFFIMER® agent can be an albumin fusion protein including at least one AFFIMER® polypeptide sequence and the sequence for mature human serum albumin (SEQ ID NO: 1096) or a variant or fragment thereof which maintains the PK and/or biodistribution properties of mature albumin to the extent desired in the fusion protein.

(SEQ ID NO: 1096)
DAHKSEVAHRFKDLGEENFKALVLIAFAQYLQQCPFEDHVKLVNEVTEF
AKTCVADESAENCDKSLHTLFGDKLCTVATLRETYGEMADCCAKQEPER
NECFLQHKDDNPNLPRLVRPEVDVMCTAFHDNEETFLKKYLYEIARRHP
YFYAPELLFFAKRYKAAFTECCQAADKAACLLPKLDELRDEGKASSAKQ
RLKCASLQKFGERAFKAWAVARLSQRFPKAEFAEVSKLVTDLTKVHTEC
CHGDLLECADDRADLAKYICENQDSISSKLKECCEKPLLEKSHCIAEVE
NDEMPADLPSLAADFVESKDVCKNYAEAKDVFLGMFLYEYARRHPDYSV
VLLLRLAKTYETTLEKCCAAADPHECYAKVFDEFKPLVEEPQNLIKQNC
ELFEQLGEYKFQNALLVRYTKKVPQVSTPTLVEVSRNLGKVGSKCCKHP
3AKRMPCAEDYLSVVLNQLCVLHEKTPVSDRVTKCCTESLVNRRPCFSA
LEVDETYVPKEFNAETFTFHADICTLSEKERQIKKQTALVELVKHKPKA
TKEQLKAVMDDFAAFVEKCCKADDKETCFAEEGKKLVAASQAALGL

The albumin sequence can be set off from the AFFIMER® polypeptide sequence or other flanking sequences in the AFFIMER® agent by use of linker sequences as described above.

While unless otherwise indicated, reference herein to “albumin” or to “mature albumin” is meant to refer to HSA. However, it is noted that full-length HSA has a signal peptide of 18 amino acids (MKWVTFISLLFLFSSAYS (SEQ ID NO: 1022)) followed by a pro-domain of 6 amino acids (RGVFRR) (SEQ ID NO: 1097); this 24 amino acid residue peptide may be referred to as the pre-pro domain. The AFFIMER® polypeptide-HSA fusion proteins can be expressed and secreted using the HSA pre-pro-domain in the recombinant proteins coding sequence. Alternatively, the AFFIMER® polypeptide-HSA fusion can be expressed and secreted through inclusion of other secretion signal sequences, such as described above.

In alternative embodiments, rather than provided as part of a fusion protein with the AFFIMER® polypeptide, the serum albumin polypeptide can be covalently coupled to the AFFIMER® polypeptide-containing polypeptide through a bond other than a backbone amide bond, such as cross-linked through chemical conjugation between amino acid sidechains on each of the albumin polypeptide and the AFFIMER® polypeptide-containing polypeptide.

### 3. Serum Binding Domains

In some embodiments, the AFFIMER® agent can include a serum-binding moiety-either as part of a fusion protein (if also a polypeptide) with the AFFIMER® polypeptide sequence or chemically conjugated through a site other than being part of a contiguous polypeptide chain.

In some embodiments, the serum-binding polypeptide is an albumin binding moiety. Albumin contains multiple hydrophobic binding pockets and naturally serves as a transporter of a variety of different ligands such as fatty acids and steroids as well as different drugs. Furthermore, the surface of albumin is negatively charged making it highly water-soluble.

The term “albumin binding moiety” as used herein refers to any chemical group capable of binding to albumin, e.g., has albumin binding affinity. Albumin binds to endogenous ligands such as fatty acids; however, it also interacts with exogenous ligands such as warfarin, penicillin and diazepam. As the binding of these drugs to albumin is reversible the albumin-drug complex serves as a drug reservoir that can enhance the drug biodistribution and bioavailability. Incorporation of components that mimic endogenous albumin-binding ligands, such as fatty acids, has been used to potentiate albumin association and increase drug efficacy.

In some embodiments, a chemical modification method that can be applied in the generation of the subject AFFIMER® agents to increase protein half-life is lipidation, which involves the covalent binding of fatty acids to peptide side chains. Originally conceived of and developed as a method for extending the half-life of insulin, lipidation shares the same basic mechanism of half-life extension as PEGylation, namely increasing the hydrodynamic radius to reduce renal filtration. However, the lipid moiety is itself relatively small and the effect is mediated indirectly through the non-covalent binding of the lipid moiety to circulating albumin. One consequence of lipidation is that it reduces the water-solubility of the peptide but engineering of the linker between the peptide and the fatty acid can modulate this, for example by the use of glutamate or mini PEGs within the linker. Linker engineering and variation of the lipid moiety can affect self-aggregation which can contribute to increased half-life by slowing down biodistribution, independent of albumin. See, for example, Jonassen et al. (2012) Pharm Res. 29 (8): 2104-14.

Other examples of albumin binding moieties for use in the generation of certain AFFIMER® agents include albumin-binding (PKE2) adnectins (See WO2011140086 “Serum Albumin Binding Molecules”, WO2015143199 “Serum albumin-binding Fibronectin Type III Domains” and WO2017053617 “Fast-off rate serum albumin binding fibronectin type iii domains”), the albumin binding domain 3 (ABD3) of protein G of Streptococcus strain G148, and the albumin binding domain antibody GSK2374697 (“AlbudAb”) or albumin binding nanobody portion of ATN-103 (Ozoralizumab).

### 4. PEGylation, XTEN, PAS and Other Polymers

A wide variety of macromolecular polymers and other molecules can be linked to the AFFIMER® polypeptides of the present disclosure to modulate biological properties of the resulting AFFIMER® agent, and/or provide new biological properties to the AFFIMER® agent. These macromolecular polymers can be linked to the AFFIMER® polypeptide via a naturally encoded amino acid, via a non-naturally encoded amino acid, or any functional substituent of a natural or non-natural amino acid, or any substituent or functional group added to a natural or non-natural amino acid. The molecular weight of the polymer may be of a wide range, including but not limited to, between about 100 Da and about 100,000 Da or more. The molecular weight of the polymer may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, the molecular weight of the polymer is between about 100 Da and about 50,000 Da. In some embodiments, the molecular weight of the polymer is between about 100 Da and about 40,000 Da. In some embodiments, the molecular weight of the polymer is between about 1,000 Da and about 40,000 Da. In some embodiments, the molecular weight of the polymer is between about 5,000 Da and about 40,000 Da. In some embodiments, the molecular weight of the polymer is between about 10,000 Da and about 40,000 Da.

For this purpose, various methods including pegylation, polysialylation, HESylation, glycosylation, or recombinant PEG analogue fused to flexible and hydrophilic amino acid chain (500 to 600 amino acids) have been developed (See Chapman, (2002) Adv Drug Deliv Rev. 54. 531-545; Schlapschy et al., (2007) Prot Eng Des Sel. 20, 273-283; Contermann (2011) Curr Op Biotechnol. 22, 868-876; Jevsevar et al., (2012) Methods Mol Biol. 901, 233-246).

Examples of polymers include but are not limited to polyalkyl ethers and alkoxy-capped analogs thereof (e.g., polyoxyethylene glycol, polyoxyethylene/propylene glycol, and methoxy or ethoxy-capped analogs thereof, especially polyoxyethylene glycol, the latter is also known as polyethylene glycol or PEG); discrete PEG (dPEG); polyvinylpyrrolidones; polyvinylalkyl ethers; polyoxazolines, polyalkyl oxazolines and polyhydroxyalkyl oxazolines; polyacrylamides, polyalkyl acrylamides, and polyhydroxyalkyl acrylamides (e.g., polyhydroxypropylmethacrylamide and derivatives thereof); polyhydroxyalkyl acrylates; polysialic acids and analogs thereof; hydrophilic peptide sequences; polysaccharides and their derivatives, including dextran and dextran derivatives, e.g., carboxymethyldextran, dextran sulfates, aminodextran; cellulose and its derivatives, e.g., carboxymethyl cellulose, hydroxyalkyl celluloses; chitin and its derivatives, e.g., chitosan, succinyl chitosan, carboxymethylchitin, carboxymethylchitosan; hyaluronic acid and its derivatives; starches; alginates; chondroitin sulfate; albumin; pullulan and carboxymethyl pullulan; polyaminoacids and derivatives thereof, e.g., polyglutamic acids, polylysines, polyaspartic acids, polyaspartamides; maleic anhydride copolymers such as: styrene maleic anhydride copolymer, divinylethyl ether maleic anhydride copolymer; polyvinyl alcohols; copolymers thereof; terpolymers thereof; mixtures thereof; and derivatives of the foregoing.

The polymer selected may be water soluble so that the AFFIMER® agent to which it is attached does not precipitate in an aqueous environment, such as a physiological environment. The water-soluble polymer may be any structural form including but not limited to linear, forked or branched. Typically, the water-soluble polymer is a poly(alkylene glycol), such as poly(ethylene glycol) (PEG), but other water-soluble polymers can also be employed. By way of example, PEG is used to describe some embodiments of this disclosure. For therapeutic use of the AFFIMER® agent, the polymer may be pharmaceutically acceptable.

The term “PEG” is used broadly to encompass any polyethylene glycol molecule, without regard to size or to modification at an end of the PEG, and can be represented as linked to the AFFIMER® polypeptide by the formula:

XO—(CH₂CH₂O)ₙ—CH₂CH₂—

or

XO—(CH₂CH₂O)ₙ—
where n is 2 to 10,000 and X is H or a terminal modification, including but not limited to, a C1-4 alkyl, a protecting group, or a terminal functional group. In some cases, a PEG used in the polypeptides of the disclosure terminates on one end with hydroxy or methoxy, e.g., X is H or CH₃ (“methoxy PEG”).

It is noted that the other end of the PEG, which is shown in the above formulas by a terminal “−”, may attach to the AFFIMER® polypeptide via a naturally-occurring or non-naturally encoded amino acid. For instance, the attachment may be through an amide, carbamate or urea linkage to an amine group (including but not limited to, the epsilon amine of lysine or the N-terminus) of the polypeptide. Alternatively, the polymer is linked by a maleimide linkage to a thiol group (including but not limited to, the thiol group of cysteine)—which in the case of attachment to the AFFIMER® polypeptide sequence per se requires altering a residue in the AFFIMER® sequence to a cysteine.

The number of water-soluble polymers linked to the AFFIMER® polypeptide (e.g., the extent of PEGylation or glycosylation) can be adjusted to provide an altered (including but not limited to, increased or decreased) pharmacologic, pharmacokinetic or pharmacodynamic characteristic such as in vivo half-life in the resulting AFFIMER® agent. In some embodiments, the half-life of the resulting AFFIMER® agent is increased at least about 10, 20, 30, 40, 50, 60, 70, 80, 90 percent, 2-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 50-fold, or at least about 100-fold over an unmodified polypeptide.

Another variation of polymer system useful to modify the PK or other biological properties of the resulting AFFIMER® agent are the use of unstructured, hydrophilic amino acid polymers that are functional analogs of PEG, particularly as part of a fusion protein with the AFFIMER® polypeptide sequence. The inherent biodegradability of the polypeptide platform makes it attractive as a potentially more benign alternative to PEG. Another advantage is the precise molecular structure of the recombinant molecule in contrast to the polydispersity of PEG. Unlike HSA and Fc peptide fusions, in which the three-dimensional folding of the fusion partner needs to be maintained, the recombinant fusions to unstructured partners can, in many cases, be subjected to higher temperatures or harsh conditions such as HPLC purification.

One of the more advanced of this class of polypeptides is termed XTEN (Amunix) and is 864 amino acids long and comprised of six amino acids (A, E, G, P, S and T). See Schellenberger et al. “A recombinant polypeptide extends the in vivo half-life of peptides and proteins in a tunable manner” 2009 Nat Biotechnol. 27 (12): 1186-90. Enabled by the biodegradable nature of the polymer, this is much larger than the 40 KDa PEGs typically used and confers a concomitantly greater half-life extension. The fusion of XTEN to the AFFIMER® polypeptide should result in halflife extension of the final AFFIMER® agent by 60- to 130-fold over the unmodified polypeptide.

A second polymer based on similar conceptual considerations is PAS (XL-Protein GmbH). Schlapschy et al. “PASYlation: a biological alternative to PEGylation for extending the plasma half-life of pharmaceutically active proteins” 2013 Protein Eng Des Sel. 26 (8): 489-501. A random coil polymer comprised of an even more restricted set of only three small uncharged amino acids, proline, alanine and serine. AS with Fc, HAS and XTEN, the PAS modification can be genetically encoded with the AFFIMER® polypeptide sequence to produce an inline fusion protein when expressed.

### D. Conjugates

The subject AFFIMER® agents may also include at least one functional moiety intended to impart detectability or additional pharmacologic activity to the AFFIMER® agent. Functional moieties for detection are those which can be employed to detect association of the AFFIMER® agent with a cell or tissue (such as a Tumor cell) in vivo. Functional moieties with pharmacologic activity are those agents which are meant to be delivered to the tissue expressing the target of the AFFIMER® agent (PD-L1 in the case of the PD-L1 AFFIMER® agents of the present disclosure) and in doing so have a pharmacologic consequence to the targeted tissues or cells.

The present disclosure provides AFFIMER® agents including conjugates of substances having a wide variety of functional groups, substituents or moieties, with those Functional Moieties including but not limited to a label; a dye; an immunoadhesion molecule; a radionuclide; a cytotoxic compound; a drug; an affinity label; a photoaffinity label; a reactive compound; a resin; a second protein or polypeptide or polypeptide analog; an antibody or antibody fragment; a metal chelator; a cofactor; a fatty acid; a carbohydrate; a polynucleotide; a DNA; a RNA; an antisense polynucleotide; a saccharide; a water-soluble dendrimer; a cyclodextrin; an inhibitory ribonucleic acid; a biomaterial; a nanoparticle; a spin label; a fluorophore, a metal-containing moiety; a radioactive moiety; a novel functional group; a group that covalently or noncovalently interacts with other molecules; a photocaged moiety; an actinic radiation excitable moiety; a photoisomerizable moiety; biotin; a derivative of biotin; a biotin analogue; a moiety incorporating a heavy atom; a chemically cleavable group; a photocleavable group; an elongated side chain; a carbon-linked sugar; a redox-active agent; an amino thioacid; a toxic moiety; an isotopically labeled moiety; a biophysical probe; a phosphorescent group; a chemiluminescent group; an electron dense group; a magnetic group; an intercalating group; a chromophore; an energy transfer agent; a biologically active agent; a detectable label; a small molecule; a quantum dot; a nanotransmitter; a radionucleotide; a radiotransmitter; a neutron-capture agent; or any combination of the above, or any other desirable compound or substance.

### 1. Labels and Detectable Moieties

Where the moiety is a detectable label, it can be a fluorescent label, radioactive label, enzymatic label or any other label known to the skilled person. In some embodiments, the Functional Moiety is a detectable label that can be included as part of a conjugate to form certain AFFIMER® agents suitable for medical imaging. By “medical imaging” is meant any technique used to visualize an internal region of the human or animal body, for the purposes of diagnosis, research or therapeutic treatment. For instance, the AFFIMER® agent can be detected (and quantitated) by radioscintigraphy, magnetic resonance imaging (MRI), computed tomography (CT scan), nuclear imaging, positron emission comprising a metal tomography (PET) contrast agent, optical imaging (such as fluorescence imaging including near-infrared fluorescence (NIRF) imaging), bioluminescence imaging, or combinations thereof. The Functional Moiety is optionally a contrast agent for X-ray imaging. Agents useful in enhancing such techniques are those materials that enable visualization of a particular locus, organ or disease site within the body, and/or that lead to some improvement in the quality of the images generated by the imaging techniques, providing improved or easier interpretation of those images. Such agents are referred to herein as contrast agents, the use of which facilitates the differentiation of different parts of the image, by increasing the “contrast” between those different regions of the image. The term “contrast agents” thus encompasses agents that are used to enhance the quality of an image that may nonetheless be generated in the absence of such an agent (as is the case, for instance, in MRI), as well as agents that are prerequisites for the generation of an image (as is the case, for instance, in nuclear imaging).

In some embodiments, the detectable label includes a chelate moiety for chelating a metal, e.g., a chelator for a radiometal or paramagnetic ion. In some embodiments, the detectable label is a chelator for a radionuclide useful for radiotherapy or imaging procedures. Radionuclides useful within the present disclosure include gamma-emitters, positron-emitters, Auger electron-emitters, X-ray emitters and fluorescence-emitters, with beta- or alpha-emitters for therapeutic use. Examples of radionuclides useful as toxins in radiation therapy include: ⁴³K, ⁴⁷Sc, ⁵¹Cr, ⁵⁷Co, ⁵⁸Co, ⁵⁹Fe, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁸¹Rb, ⁹⁰Y, ⁹⁷Ru, ^{99m}Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Conditions under which a chelator will coordinate a metal are described, for example, by Gansow et al., U.S. Pat. Nos. 4,831,175, 4,454,106 and 4,472,509. Examples of chelators includes, merely to illustrate, 1,4,7-triazacyclononane-N,N′,N″-triacetic acid (NOTA) 1,4,7,10-tetraazacyclododecane-N,N′,N″,N′″-tetraacetic acid (DOTA) 1,4,8,11-tetraazacyclotetradecane-N,N′,N″,N′″-tetraacetic acid (TETA).

Other detectable isotopes that can be incorporated directly into the amino acid residues of the AFFIMER® polypeptide or which otherwise do not require a chelator, include ³H, ¹⁴C, ³²P, ³⁵S and ³⁶Cl.

Paramagnetic ions, useful for diagnostic procedures, may also be administered. Examples of paramagnetic ions include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), erbium (III), or combinations of these paramagnetic ions.

Examples of fluorescent labels include, but are not restricted to, organic dyes (e.g., cyanine, fluorescein, rhodamine, Alexa Fluors, Dylight fluors, ATTO Dyes, BODIPY Dyes, etc.), biological fluorophores (e.g., green fluorescent protein (GFP), R-Phycoerythrin, etc.), and quantum dots.

Non-limiting fluorescent compound that may be used in the present disclosure include, Cγ5, Cγ5.5 (also known as Cγ5++), Cγ2, fluorescein isothiocyanate (FITC), tetramethylrhodamine isothiocyanate (TRITC), phycoerythrin, Cγ7, fluorescein (FAM), Cγ3, Cγ3.5 (also known as Cγ3++), Texas Red, LightCycler-Red 640, LightCycler Red 705, tetramethylrhodamine (TMR), rhodamine, rhodamine derivative (ROX), hexachlorofluorescein (HEX), rhodamine 6G (R6G), the rhodamine derivative JA133, Alexa Fluorescent Dyes (such as Alexa Fluor 488, Alexa Fluor 546, Alexa Fluor 633, Alexa Fluor 555, and Alexa Fluor 647), 4′,6-diamidino-2-phenylindole (DAPI), Propidium iodide, AMCA, Spectrum Green, Spectrum Orange, Spectrum Aqua, Lissamine, and fluorescent transition metal complexes, such as europium. Fluorescent compound that can be used also include fluorescent proteins, such as GFP (green fluorescent protein), enhanced GFP (EGFP), blue fluorescent protein and derivatives (BFP, EBFP, EBFP2, Azurite, mKalama1), cyan fluorescent protein and derivatives (CFP, ECFP, Cerulean, CyPet) and yellow fluorescent protein and derivatives (YFP, Citrine, Venus, YPet). WO2008142571, WO2009056282, WO9922026.

Examples of enzymatic labels include, but are not restricted to, horseradish peroxidase (HRP), alkaline phosphatase (AP), glucose oxidase and β-galactosidase.

Another well-known label is biotin. Biotin labels are typically composed of the biotinyl group, a spacer arm and a reactive group that is responsible for attachment to target functional groups on proteins. Biotin can be useful for attaching the labelled protein to other moieties which comprise an avidin moiety.

### 2. AFFIMER® Polypeptide-Drug Conjugates

In some embodiments, the AFFIMER® agent includes at least one therapeutic agent, e.g., to form an AFFIMER® polypeptide-drug conjugate. As used herein, the term “therapeutic agent” refers to a substance that may be used in the cure, mitigation, treatment, or prevention of disease in a human or another animal. Such therapeutic agents include substances recognized in the official United States Pharmacopeia, official Homeopathic Pharmacopeia of the United States, official National Formulary, or any supplement thereof, and include but are not limited to small molecules, nucleotides, oligopeptides, polypeptides, etc. Therapeutic agents that may be attached to AFFIMER® polypeptides include but are not limited to, cytotoxic agents, anti-metabolites, alkylating agents, antibiotics, growth factor, cytokines, anti-angiogenic agents, antimitotic agents, toxins, apoptotic agents or the like, such as DNA alkylating agents, topoisomerase inhibitors, microtubule inhibitors (e.g., DM1, DM4, MMAF and MMAE), endoplasmic reticulum stress inducing agents, platinum compounds, antimetabolites, vincalkaloids, taxanes, epothilones, enzyme inhibitors, receptor antagonists, therapeutic antibodies, tyrosine kinase inhibitors, radiosensitizers, and chemotherapeutic combination therapies, such as illustrations.

Non-limiting examples of DNA alkylating agents are nitrogen mustards, such as Mechlorethamine, Cyclophosphamide (Ifosfamide, Trofosfamide), Chlorambucil (Melphalan, Prednimustine), Bendamustine, Uramustine and Estramustine; nitrosoureas, such as Carmustine (BCNU), Lomustine (Semustine), Fotemustine, Nimustine, Ranimustine and Streptozocin; alkyl sulfonates, such as Busulfan (Mannosulfan, Treosulfan); Aziridines, such as Carboquone, ThioTEPA, Triaziquone, Triethylenemelamine; Hydrazines (Procarbazine); Triazenes such as Dacarbazine and Temozolomide; Altretamine and Mitobronitol.

Non-limiting examples of Topoisomerase I inhibitors include Campothecin derivatives including CPT-11 (irinotecan), SN-38, APC, NPC, campothecin, topotecan, exatecan mesylate, 9-nitrocamptothecin, 9-aminocamptothecin, lurtotecan, rubitecan, silatecan, gimatecan, diflomotecan, extatecan, BN-80927, DX-8951f, and MAG-CPT as described in Pommier Y. (2006) Nat. Rev. Cancer 6 (10): 789-802 and U.S. Patent Publication No. 200510250854; Protoberberine alkaloids and derivatives thereof including berberrubine and coralyne as described in Li et al. (2000) Biochemistry 39 (24): 7107-7116 and Gatto et al. (1996) Cancer Res. 15 (12): 2795-2800; Phenanthroline derivatives including Benzo[i]phenanthridine, Nitidine, and fagaronine as described in Makhey et al. (2003) Bioorg. Med. Chem. 11 (8): 1809-1820; Terbenzimidazole and derivatives thereof as described in Xu (1998) Biochemistry 37 (10): 3558-3566; and Anthracycline derivatives including Doxorubicin, Daunorubicin, and Mitoxantrone as described in Foglesong et al. (1992) Cancer Chemother. Pharmacol. 30 (2): 123-125, Crow et al. (1994) J. Med. Chem. 37 (19): 31913194, and Crespi et al. (1986) Biochem. Biophys. Res. Commun. 136 (2): 521-8. Topoisomerase II inhibitors include but are not limited to Etoposide and Teniposide. Dual topoisomerase I and II inhibitors include but are not limited to, Saintopin and other Naphthecenediones, DACA and other Acridine-4-Carboxamindes, Intoplicine and other Benzopyridoindoles, TAS-103 and other 7H-indeno[2,1-c]Quinoline-7-ones, Pyrazoloacridine, XR 11576 and other Benzophenazines, XR 5944 and other Dimeric compounds, 7-oxo-7H-dibenz[f,ij]Isoquinolines and 7-oxo-7H-benzo[e]Perimidines, and Anthracenyl-amino Acid Conjugates as described in Denny and Baguley (2003) Curr. Top. Med. Chem. 3 (3): 339-353. Some agents inhibit Topoisomerase II and have DNA intercalation activity such as, but not limited to, Anthracyclines (Aclarubicin, Daunorubicin, Doxorubicin, Epirubicin, Idarubicin, Amrubicin, Pirarubicin, Valrubicin, Zorubicin) and Antracenediones (Mitoxantrone and Pixantrone).

Non-limiting examples of DNA synthesis inhibitors include Calicheamicin, Doxorubicin, Duocarmycin, and PBD.

Non-limiting examples of microtubule inhibitors include DM1, DM4, MMAF, and MMAE.

Examples of endoplasmic reticulum stress inducing agents include but are not limited to, dimethyl-celecoxib (DMC), nelfinavir, celecoxib, and boron radiosensitizers (e.g., velcade (Bortezomib)).

Non-limiting examples of platinum-based compound include Carboplatin, Cisplatin, Nedaplatin, Oxaliplatin, Triplatin tetranitrate, Satraplatin, Aroplatin, Lobaplatin, and JM-216. (see Mckeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

Non-limiting examples of antimetabolite agents include Folic acid based, e.g. dihydrofolate reductase inhibitors, such as Aminopterin, Methotrexate and Pemetrexed; thymidylate synthase inhibitors, such as Raltitrexed, Pemetrexed; Purine based, e.g. an adenosine deaminase inhibitor, such as Pentostatin, a thiopurine, such as Thioguanine and Mercaptopurine, a halogenated/ribonucleotide reductase inhibitor, such as Cladribine, Clofarabine, Fludarabine, or a guanine/guanosine: thiopurine, such as Thioguanine; or Pyrimidine based, e.g. cytosine/cytidine: hypomethylating agent, such as Azacitidine and Decitabine, a DNA polymerase inhibitor, such as Cytarabine, a ribonucleotide reductase inhibitor, such as Gemcitabine, or a thymine/thymidine: thymidylate synthase inhibitor, such as a Fluorouracil (5-FU). Equivalents to 5-FU include prodrugs, analogs and derivative thereof such as 5′-deoxy-5-fluorouridine (doxifluoroidine), 1-tetrahydrofuranyl-5-fluorouracil (ftorafur), Capecitabine (Xeloda), S-I (MBMS-247616, consisting of tegafur and two modulators, a 5-chloro-2,4-dihydroxypyridine and potassium oxonate), ralititrexed (tomudex), no latrexed (Thymitaq, AG337), LY231514 and ZD9331, as described for example in Papamicheal (1999) The Oncologist 4:478-487.

Examples of vincalkaloids, include but are not limited to Vinblastine, Vincristine, Vinflunine, Vindesine and Vinorelbine.

Examples of taxanes include but are not limited to docetaxel, Larotaxel, Ortataxel, Paclitaxel and Tesetaxel. An example of an epothilone is iabepilone.

Examples of enzyme inhibitors include but are not limited to farnesyltransferase inhibitors (Tipifamib); CDK inhibitor (Alvocidib, Seliciclib); proteasome inhibitor (Bortezomib); phosphodiesterase inhibitor (Anagrelide; rolipram); IMP dehydrogenase inhibitor (Tiazofurine); and lipoxygenase inhibitor (Masoprocol). Examples of receptor antagonists include but are not limited to ERA (Atrasentan); retinoid X receptor (Bexarotene); and a sex steroid (Testolactone).

Examples of therapeutic antibodies include but are not limited to anti-HER1/EGFR (Cetuximab, Panitumumab); Anti-HER2/neu (erbB2) receptor (Trastuzumab); Anti-EpCAM (Catumaxomab, Edrecolomab) Anti-VEGF-A (Bevacizumab); Anti-CD20 (Rituximab, Tositumomab, Ibritumomab); Anti-CD52 (Alemtuzumab); and Anti-CD33 (Gemtuzumab). U.S. Pat. Nos. 5,776,427 and 7,601,355.

Examples of tyrosine kinase inhibitors include but are not limited to inhibitors to ErbB: HER1/EGFR (Erlotinib, Gefitinib, Lapatinib, Vandetanib, Sunitinib, Neratinib); HER2/neu (Lapatinib, Neratinib); RTK class III: C-kit (Axitinib, Sunitinib, Sorafenib), FLT3 (Lestaurtinib), PDGFR (Axitinib, Sunitinib, Sorafenib); and VEGFR (Vandetanib, Semaxanib, Cediranib, Axitinib, Sorafenib); bcr-abl (Imatinib, Nilotinib, Dasatinib); Src (Bosutinib) and Janus kinase 2 (Lestaurtinib).

Chemotherapeutic agents that can be attached to the present AFFIMER® polypeptides may also include amsacrine, Trabectedin, retinoids (Alitretinoin, Tretinoin), Arsenic trioxide, asparagine depleter Asparaginase/Pegaspargase), Celecoxib, Demecolcine, Elesclomol, Elsamitrucin, Etoglucid, Lonidamine, Lucanthone, Mitoguazone, Mitotane, Oblimersen, Temsirolimus, and Vorinostat.

Examples of specific therapeutic agents that can be linked, ligated, or associated with the AFFIMER® polypeptides of the disclosure are flomoxef; fortimicin(s); gentamicin(s); glucosulfone solasulfone; gramicidin S; gramicidin(s); grepafloxacin; guamecycline; hetacillin; isepamicin; josamycin; kanamycin(s); flomoxef; fortimicin(s); gentamicin(s); glucosulfone solasulfone; gramicidin S; gramicidin(s); grepafloxacin; guamecycline; hetacillin; isepamicin; josamycin; kanamycin(s); bacitracin; bambermycin(s); biapenem; brodimoprim; butirosin; capreomycin; carbenicillin; carbomycin; carumonam; cefadroxil; cefamandole; cefatrizine; cefbuperazone; cefclidin; cefdinir; cefditoren; cefepime; cefetamet; cefixime; cefinenoxime; cefininox; cladribine; apalcillin; apicycline; apramycin; arbekacin; aspoxicillin; azidamfenicol; aztreonam; cefodizime; cefonicid; cefoperazone; ceforamide; cefotaxime; cefotetan; cefotiam; cefozopran; cefpimizole; cefpiramide; cefpirome; cefprozil; cefroxadine; cefteram; ceftibuten; cefuzonam; cephalexin; cephaloglycin; cephalosporin C; cephradine; chloramphenicol; chlortetracycline; clinafloxacin; clindamycin; clomocycline; colistin; cyclacillin; dapsone; demeclocycline; diathymosulfone; dibekacin; dihydrostreptomycin; 6-mercaptopurine; thioguanine; capecitabine; docetaxel; etoposide; gemcitabine; topotecan; vinorelbine; vincristine; vinblastine; teniposide; melphalan; methotrexate; 2-p-sulfanilyanilinoethanol; 4,4′-sulfinyldianiline; 4-sulfanilamidosalicylic acid; butorphanol; nalbuphine. streptozocin; doxorubicin; daunorubicin; plicamycin; idarubicin; mitomycin C; pentostatin; mitoxantrone; cytarabine; fludarabine phosphate; butorphanol; nalbuphine. streptozocin; doxorubicin; daunorubicin; plicamycin; idarubicin; mitomycin C; pentostatin; mitoxantrone; cytarabine; fludarabine phosphate; acediasulfone; acetosulfone; amikacin; amphotericin B; ampicillin; atorvastatin; enalapril; ranitidine; ciprofloxacin; pravastatin; clarithromycin; cyclosporin; famotidine; leuprolide; acyclovir; paclitaxel; azithromycin; lamivudine; budesonide; albuterol; indinavir; metformin; alendronate; nizatidine; zidovudine; carboplatin; metoprolol; amoxicillin; diclofenac; lisinopril; ceftriaxone; captopril; salmeterol; xinafoate; imipenem; cilastatin; benazepril; cefaclor; ceftazidime; morphine; dopamine; bialamicol; fluvastatin; phenamidine; podophyllinic acid 2-ethylhydrazine; acriflavine; chloroazodin; arsphenamine; amicarbilide; aminoquinuride; quinapril; oxymorphone; buprenorphine; floxuridine; dirithromycin; doxycycline; enoxacin; enviomycin; epicillin; erythromycin; leucomycin(s); lincomycin; lomefloxacin; lucensomycin; lymecycline; meclocycline; meropenem; methacycline; micronomicin; midecamycin(s); minocycline; moxalactam; mupirocin; nadifloxacin; natamycin; neomycin; netilmicin; norfloxacin; oleandomycin; oxytetracycline; p-sulfanilylbenzylamine; panipenem; paromomycin; pazufloxacin; penicillin N; pipacycline; pipemidic acid; polymyxin; primycin; quinacillin; ribostamycin; rifamide; rifampin; rifamycin SV; rifapentine; rifaximin; ristocetin; ritipenem; rokitamycin; rolitetracycline; rosaramycin; roxithromycin; salazosulfadimidine; sancycline; sisomicin; sparfloxacin; spectinomycin; spiramycin; streptomycin; succisulfone; sulfachrysoidine; sulfaloxic acid; sulfamidochrysoidine; sulfanilic acid; sulfoxone; teicoplanin; temafloxacin; temocillin; tetroxoprim; thiamphenicol; thiazolsulfone; thiostrepton; ticarcillin; tigemonam; tobramycin; tosufloxacin; trimethoprim; trospectomycin; trovafloxacin; tuberactinomycin; vancomycin; azaserine; candicidin(s); chlorphenesin; dermostatin(s); filipin; fungichromin; mepartricin; nystatin; oligomycin(s); perimycin A; tubercidin; 6-azauridine; 6-diazo-5-oxo-L-norleucine; aclacinomycin(s); ancitabine; anthramycin; azacitadine; azaserine; bleomycin(s); ethyl biscoumacetate; ethylidene dicoumarol; iloprost; lamifiban; taprostene; tioclomarol; tirofiban; amiprilose; bucillamine; gusperimus; gentisic acid; glucamethacin; glycol salicylate; meclofenamic acid; mefenamic acid; mesalamine; niflumic acid; olsalazine; oxaceprol; S-enosylmethionine; salicylic acid; salsalate; sulfasalazine; tolfenamic acid; carubicin; carzinophillin A; chlorozotocin; chromomycin(s); denopterin; doxifluridine; edatrexate; eflornithine; elliptinium; enocitabine; epirubicin; mannomustine; menogaril; mitobronitol; mitolactol; mopidamol; mycophenolic acid; nogalamycin; olivomycin(s); peplomycin; pirarubicin; piritrexim; prednimustine; procarbazine; pteropterin; puromycin; ranimustine; streptonigrin; thiamiprine; mycophenolic acid; procodazole; romurtide; sirolimus (rapamycin); tacrolimus; butethamine; fenalcomine; hydroxytetracaine; naepaine; orthocaine; piridocaine; salicyl alcohol; 3-amino-4-hydroxybutyric acid; aceclofenac; alminoprofen; amfenac; bromfenac; bromosaligenin; bumadizon; carprofen; diclofenac; diflunisal; ditazol; enfenamic acid; etodolac; etofenamate; fendosal; fepradinol; flufenamic acid; Tomudex (N-[[5-[[(1,4-Dihydro-2-methyl-4-oxo-6-quinazolinyl)methyl]methylamino]-2-thienyl]carbonyl]-L-glutamic acid), trimetrexate, tubercidin, ubenimex, vindesine, zorubicin; argatroban; coumetarol or dicoumarol.

In some embodiments, the AFFIMER® agent includes a conjugated cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

Any method known in the art for conjugating to antibodies and other proteins may be employed in generating the conjugates of the present disclosure, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating peptide, polypeptide and organic and inorganic moieties to antibodies and other proteins are conventional and very well known in the art and readily adapted for generating those versions of the subject AFFIMER® agents.

Where the conjugated moiety is a peptide or polypeptide, that moiety can be chemically cross-linked to the AFFIMER® polypeptide or can be included as part of a fusion protein with the AFFIMER® polypeptide. And illustrative example would be a diptheria toxin-AFFIMER® fusion protein. In the case of non-peptide entities, the addition to the AFFIMER® polypeptide will generally be by way of chemical conjugation to the AFFIMER® polypeptide-such as through a functional group on an amino acid side chain or the carboxyl group at the C-terminal or amino group at the N-terminal end of the polypeptide. In some embodiment, whether as a fusion protein or chemically cross-linked moiety, the conjugated moiety will include at least one site that can be cleaved by an enzyme or are otherwise sensitive to an environmental condition (such as pH) that permits the conjugated moiety to be released from the AFFIMER® polypeptide, such as in the tumor or other diseased tissue (or tissue to be protected if the conjugated moiety functions to protect healthy tissue).

### a) Enzyme-Cleavable Linkers

An AFFIMER® polypeptide-drug conjugate, in some embodiments, comprises an enzyme-cleavable linker, which links the half-life extension moiety to a drug moiety. The linker (e.g., the substrate recognition sequence (SRS) of the linker) is selectively cleaved in the vicinity of the target cells so that the free drug moiety is released from the conjugate in the vicinity of the target cells so as to exert its pharmacological activities preferentially on the cells/tissue nearby to the target cells, rather than on wanted (healthy) cells. Thus, in some embodiments, the SRS is selectively cleaved such that the drug moiety is released as the free drug moiety in the vicinity of the target cells at least five times or ten times more than the extent to which the free drug moiety it is released in the vicinity of healthy cells/tissues, and in some embodiments, at least 100 or 500 or 1000 times more.

For a given target cell, the skilled person will be able to identify appropriate SRS that is selectively cleavable in the vicinity of the target cell, using established methods in the art. For example, which proteases cleave which peptides can be assessed by consulting peptide libraries and studying an MS analysis of the fragmentation profile following cleavage. Also, published literature of protease cleavage motifs and peptide cleavage data can be searched as described further below.

In some aspects, the SRS is a protease cleavage site. Thus, when the target cells are tumor cells, the SRS may be cleavable selectively by proteases that reside in the vicinity of the tumor cells. Thus, the SRS may be one that is cleavable by a tumor associated protease. It is well known that during tumor development, tumors aberrantly express proteases which allow them to invade local tissues and eventually metastasize.

For example, the protease may be present extracellularly in the diseased state tissue in a subject at levels at least 2, 3, 4, 5, 6, 7, 8, 9, 10, 15, 20, 30, 40, 50, 60, 70, 80, 90, or 100 times greater than the healthy state of the tissue in the subject.

As another example, the protease may be present extracellularly in the diseased state of the tissue in a subject at levels at least 2, 3, 4, 5, 6, 7, 8, 9, 10, 15, 20, 30, 40, 50, 60, 70, 80, 90 or 100 times greater than other tissue of the subject.

In some embodiments, the protease is a serine protease, metal protease, or cysteine protease.

The protease may be a metalloproteinase (MMP1-28) including both membrane bound (MMP14-17 and MMP24-25) and secreted forms (MMP1-13 and MMP 18-23 and MMP26-28). The protease may belong to the A Disintegrin and Metalloproteinase (ADAM) and A Disintegrin, or Metalloproteinase with Thrombospondin Motifs (ADAMTS) families of proteases. Other examples include CD 10 (CALLA) and prostate specific antigen (PSA). It is appreciated that the proteases may or may not be membrane bound.

Protease cleavage sites are well known in the scientific literature, and can readily serve as the basis for a given SRS being included in the drug-conjugate moieties using established synthetic techniques known in the art.

To the extent representing a protease whose extracellular concentration is upregulated/increased in the target tissue by changes in expression, cellular trafficking or, in the case of intracellular enzymes that may become extracellular, by cell lysis caused by the disease state, SRS may utilized which are designed to be selectively cleavable by one or a select subgroup of human proteases selected from the group consisting of (MEROPS peptidase database number provided in parentheses; Rawlings N. D., Morton F. R., Kok, C. Y., Kong, J. & Barrett A. J. (2008) MEROPS: the peptidase database. Nucleic Acids Res. 36 Database issue, D320-325): pepsin A (MER000885), gastricsin (MER000894), memapsin-2 (MER005870), renin (MER000917), cathepsin D (MER000911), cathepsin E (MER000944), memapsin-1 (MER005534), napsin A (MER004981), Mername-AA034 peptidase (MER014038), pepsin A4 (MER037290), pepsin A5 (Homo sapiens) (MER037291), hCG1733572 (Homo sapiens)-type putative peptidase (MER107386), napsin B pseudogene (MER004982), CYMP g.p. (Homo sapiens) (MER002929), subfamily A1A unassigned peptidases (MER181559), mouse mammary tumor virus retropepsin (MER048030), rabbit endogenous retrovirus endopeptidase (MER043650), S71-related human endogenous retropepsin (MER001812), RTVL-H-type putative peptidase (MER047117), RTVL-H-type putative peptidase (MER047133), RTVL-H-type putative peptidase (MER047160), RTVL-H-type putative peptidase (MER047206), RTVL-H-type putative peptidase (MER047253), RTVL-H-type putative peptidase (MER047260), RTVL-H-type putative peptidase (MER047291), RTVL-H-type putative peptidase (MER047418), RTVL-H-type putative peptidase (MER047440), RTVL-H-type putative peptidase (MER047479), RTVL-H-type putative peptidase (MER047559), RTVL-H-type putative peptidase (MER047583), RTVL-H-type putative peptidase (MERO 15446), human endogenous retrovirus retropepsin homologue 1 (MERO 15479), human endogenous retrovirus retropepsin homologue 2 (MERO 15481), endogenous retrovirus retropepsin pseudogene 1 (Homo sapiens chromosome 14) (MER029977), endogenous retrovirus retropepsin pseudogene 2 (Homo sapiens chromosome 8) (MER029665), endogenous retrovirus retropepsin pseudogene 3 (Homo sapiens chromosome 17) (MER002660), endogenous retrovirus retropepsin pseudogene 3 (Homo sapiens chromosome 17) (MER030286), endogenous retrovirus retropepsin pseudogene 3 (Homo sapiens chromosome 17) (MER047144), endogenous retrovirus retropepsin pseudogene 5 (Homo sapiens chromosome 12) (MER029664), endogenous retrovirus retropepsin pseudogene 6 (Homo sapiens chromosome 7) (MER002094), endogenous retrovirus retropepsin pseudogene 7 (Homo sapiens chromosome 6) (MER029776), endogenous retrovirus retropepsin pseudogene 8 (Homo sapiens chromosome Y) (MER030291), endogenous retrovirus retropepsin pseudogene 9 (Homo sapiens chromosome 19) (MER029680), endogenous retrovirus retropepsin pseudogene 10 (Homo sapiens chromosome 12) (MER002848), endogenous retrovirus retropepsin pseudogene 11 (Homo sapiens chromosome 17) (MER004378), endogenous retrovirus retropepsin pseudogene 12 (Homo sapiens chromosome 11) (MER003344), endogenous retrovirus retropepsin pseudogene 13 (Homo sapiens chromosome 2 and similar) (MER029779), endogenous retrovirus retropepsin pseudogene 14 (Homo sapiens chromosome 2) (MER029778), endogenous retrovirus retropepsin pseudogene 15 (Homo sapiens chromosome 4) (MER047158), endogenous retrovirus retropepsin pseudogene 15 (Homo sapiens chromosome 4) (MER047332), endogenous retrovirus retropepsin pseudogene 15 (Homo sapiens chromosome 4) (MER003182), endogenous retrovirus retropepsin pseudogene 16 (MER047165), endogenous retrovirus retropepsin pseudogene 16 (MER047178), endogenous retrovirus retropepsin pseudogene 16 (MER047200), endogenous retrovirus retropepsin pseudogene 16 (MER047315), endogenous retrovirus retropepsin pseudogene 16 (MER047405), endogenous retrovirus retropepsin pseudogene 16 (MER030292), endogenous retrovirus retropepsin pseudogene 17 (Homo sapiens chromosome 8) (MER005305), endogenous retrovirus retropepsin pseudogene 18 (Homo sapiens chromosome 4) (MER030288), endogenous retrovirus retropepsin pseudogene 19 (Homo sapiens chromosome 16) (MER001740), endogenous retrovirus retropepsin pseudogene 21 (Homo sapiens) (MER047222), endogenous retrovirus retropepsin pseudogene 21 (Homo sapiens) (MER047454), endogenous retrovirus retropepsin pseudogene 21 (Homo sapiens) (MER047477), endogenous retrovirus retropepsin pseudogene 21 (Homo sapiens) (MER004403), endogenous retrovirus retropepsin pseudogene 22 (Homo sapiens chromosome X) (MER030287), subfamily A2A non peptidase homologues (MER047046), subfamily A2A non-peptidase homologues (MER047052), subfamily A2A non-peptidase homologues (MER047076), subfamily A2A non-peptidase homologues (MER047080), subfamily A2A non-peptidase homologues (MER047088), subfamily A2A non-peptidase homologues (MER047089), subfamily A2A non-peptidase homologues (MER047091), subfamily A2A non-peptidase homologues (MER047092), subfamily A2A non-peptidase homologues (MER047093), subfamily A2A non-peptidase homologues (MER047094), subfamily A2A non-peptidase homologues (MER047097), subfamily A2A non-peptidase homologues (MER047099), subfamily A2A non-peptidase homologues (MER047101), subfamily A2A non-peptidase homologues (MER047102), subfamily A2A non-peptidase homologues (MER047107), subfamily A2A non-peptidase homologues (MER047108), subfamily A2A non-peptidase homologues (MER047109), subfamily A2A non-peptidase homologues (MER047110), subfamily A2A non-peptidase homologues (MER047111), subfamily A2A non-peptidase homologues (MER047114), subfamily A2A non-peptidase homologues (MER047118), subfamily A2A non-peptidase homologues (MER047121), subfamily A2A non-peptidase homologues (MER047122), subfamily A2A non-peptidase homologues (MER047126), subfamily A2A non-peptidase homologues (MER047129), subfamily A2A non-peptidase homologues (MER047130), subfamily A2A non-peptidase homologues (MER047134), subfamily A2A non-peptidase homologues (MER047135), subfamily A2A non-peptidase homologues (MER047137), subfamily A2A non-peptidase homologues (MER047140), subfamily A2A non-peptidase homologues (MER047141), subfamily A2A non-peptidase homologues (MER047142), subfamily A2A non-peptidase homologues (MER047148), subfamily A2A non-peptidase homologues (MER047149), subfamily A2A non-peptidase homologues (MER047151), subfamily A2A non-peptidase homologues (MER047154), subfamily A2A non-peptidase homologues (MER047155), subfamily A2A non-peptidase homologues (MER047156), subfamily A2A non-peptidase homologues (MER047157), subfamily A2A non-peptidase homologues (MER047159), subfamily A2A non-peptidase homologues (MER047161), subfamily A2A non-peptidase homologues (MER047163), subfamily A2A non-peptidase homologues (MER047166), subfamily A2A non-peptidase homologues (MER047171), subfamily A2A non-peptidase homologues (MER047173), subfamily A2A non-peptidase homologues (MER047174), subfamily A2A non-peptidase homologues (MER047179), subfamily A2A non-peptidase homologues (MER047183), subfamily A2A non-peptidase homologues (MER047186), subfamily A2A non-peptidase homologues (MER047190), subfamily A2A non-peptidase homologues (MER047191), subfamily A2A non-peptidase homologues (MER047196), subfamily A2A non-peptidase homologues (MER047198), subfamily A2A non-peptidase homologues (MER047199), subfamily A2A non-peptidase homologues (MER047201), subfamily A2A non-peptidase homologues (MER047202), subfamily A2A non-peptidase homologues (MER047203), subfamily A2A non-peptidase homologues (MER047204), subfamily A2A non-peptidase homologues (MER047205), subfamily A2A non-peptidase homologues (MER047207), subfamily A2A non-peptidase homologues (MER047208), subfamily A2A non-peptidase homologues (MER047210), subfamily A2A non-peptidase homologues (MER047211), subfamily A2A non-peptidase homologues (MER047212), subfamily A2A non-peptidase homologues (MER047213), subfamily A2A non-peptidase homologues (MER047215), subfamily A2A non-peptidase homologues (MER047216), subfamily A2A non-peptidase homologues (MER047218), subfamily A2A non-peptidase homologues (MER047219), subfamily A2A non-peptidase homologues (MER047221), subfamily A2A non-peptidase homologues (MER047224), subfamily A2A non-peptidase homologues (MER047225), subfamily A2A non-peptidase homologues (MER047226), subfamily A2A non-peptidase homologues (MER047227), subfamily A2A non-peptidase homologues (MER047230), subfamily A2A non-peptidase homologues (MER047232), subfamily A2A non-peptidase homologues (MER047233), subfamily A2A non-peptidase homologues (MER047234), subfamily A2A non-peptidase homologues (MER047236), subfamily A2A non-peptidase homologues (MER047238), subfamily A2A non-peptidase homologues (MER047239), subfamily A2A non-peptidase homologues (MER047240), subfamily A2A non-peptidase homologues (MER047242), subfamily A2A non-peptidase homologues (MER047243), subfamily A2A non-peptidase homologues (MER047249), subfamily A2A non-peptidase homologues (MER047251), subfamily A2A non-peptidase homologues (MER047252), subfamily A2A non-peptidase homologues (MER047254), subfamily A2A non-peptidase homologues (MER047255), subfamily A2A non-peptidase homologues (MER047263), subfamily A2A non-peptidase homologues (MER047265), subfamily A2A non-peptidase homologues (MER047266), subfamily A2A non-peptidase homologues (MER047267), subfamily A2A non-peptidase homologues (MER047268), subfamily A2A non-peptidase homologues (MER047269), subfamily A2A non-peptidase homologues (MER047272), subfamily A2A non-peptidase homologues (MER047273), subfamily A2A non-peptidase homologues (MER047274), subfamily A2A non-peptidase homologues (MER047275), subfamily A2A non-peptidase homologues (MER047276), subfamily A2A non-peptidase homologues (MER047279), subfamily A2A non-peptidase homologues (MER047280), subfamily A2A non-peptidase homologues (MER047281), subfamily A2A non-peptidase homologues (MER047282), subfamily A2A non-peptidase homologues (MER047284), subfamily A2A non-peptidase homologues (MER047285), subfamily A2A non-peptidase homologues (MER047289), subfamily A2A non-peptidase homologues (MER047290), subfamily A2A non-peptidase homologues (MER047294), subfamily A2A non-peptidase homologues (MER047295), subfamily A2A non-peptidase homologues (MER047298), subfamily A2A non-peptidase homologues (MER047300), subfamily A2A non-peptidase homologues (MER047302), subfamily A2A non-peptidase homologues (MER047304), subfamily A2A non-peptidase homologues (MER047305), subfamily A2A non-peptidase homologues (MER047306), subfamily A2A non-peptidase homologues (MER047307), subfamily A2A non-peptidase homologues (MER047310), subfamily A2A non-peptidase homologues (MER047311), subfamily A2A non-peptidase homologues (MER047314), subfamily A2A non-peptidase homologues (MER047318), subfamily A2A non-peptidase homologues (MER047320), subfamily A2A non-peptidase homologues (MER047321), subfamily A2A non-peptidase homologues (MER047322), subfamily A2A non-peptidase homologues (MER047326), subfamily A2A non-peptidase homologues (MER047327), subfamily A2A non-peptidase homologues (MER047330), subfamily A2A non-peptidase homologues (MER047333), subfamily A2A non-peptidase homologues (MER047362), subfamily A2A non-peptidase homologues (MER047366), subfamily A2A non-peptidase homologues (MER047369), subfamily A2A non-peptidase homologues (MER047370), subfamily A2A non-peptidase homologues (MER047371), subfamily A2A non-peptidase homologues (MER047375), subfamily A2A non-peptidase homologues (MER047376), subfamily A2A non-peptidase homologues (MER047381), subfamily A2A non-peptidase homologues (MER047383), subfamily A2A non-peptidase homologues (MER047384), subfamily A2A non-peptidase homologues (MER047385), subfamily A2A non-peptidase homologues (MER047388), subfamily A2A non-peptidase homologues (MER047389), subfamily A2A non-peptidase homologues (MER047391), subfamily A2A non-peptidase homologues (MER047394), subfamily A2A non-peptidase homologues (MER047396), subfamily A2A non-peptidase homologues (MER047400), subfamily A2A non-peptidase homologues (MER047401), subfamily A2A non-peptidase homologues (MER047403), subfamily A2A non-peptidase homologues (MER047406), subfamily A2A non-peptidase homologues (MER047407), subfamily A2A non-peptidase homologues (MER047410), subfamily A2A non-peptidase homologues (MER047411), subfamily A2A non-peptidase homologues (MER047413), subfamily A2A non-peptidase homologues (MER047414), subfamily A2A non-peptidase homologues (MER047416), subfamily A2A non-peptidase homologues (MER047417), subfamily A2A non-peptidase homologues (MER047420), subfamily A2A non-peptidase homologues (MER047423), subfamily A2A non-peptidase homologues (MER047424), subfamily A2A non-peptidase homologues (MER047428), subfamily A2A non-peptidase homologues (MER047429), subfamily A2A non-peptidase homologues (MER047431), subfamily A2A non-peptidase homologues (MER047434), subfamily A2A non-peptidase homologues (MER047439), subfamily A2A non-peptidase homologues (MER047442), subfamily A2A non-peptidase homologues (MER047445), subfamily A2A non-peptidase homologues (MER047449), subfamily A2A non-peptidase homologues (MER047450), subfamily A2A non-peptidase homologues (MER047452), subfamily A2A non-peptidase homologues (MER047455), subfamily A2A non-peptidase homologues (MER047457), subfamily A2A non-peptidase homologues (MER047458), subfamily A2A non-peptidase homologues (MER047459), subfamily A2A non-peptidase homologues (MER047463), subfamily A2A non-peptidase homologues (MER047468), subfamily A2A non-peptidase homologues (MER047469), subfamily A2A non-peptidase homologues (MER047470), subfamily A2A non-peptidase homologues (MER047476), subfamily A2A non-peptidase homologues (MER047478), subfamily A2A non-peptidase homologues (MER047483), subfamily A2A non-peptidase homologues (MER047488), subfamily A2A non-peptidase homologues (MER047489), subfamily A2A non-peptidase homologues (MER047490), subfamily A2A non-peptidase homologues (MER047493), subfamily A2A non-peptidase homologues (MER047494), subfamily A2A non-peptidase homologues (MER047495), subfamily A2A non-peptidase homologues (MER047496), subfamily A2A non-peptidase homologues (MER047497), subfamily A2A non-peptidase homologues (MER047499), subfamily A2A non-peptidase homologues (MER047502), subfamily A2A non-peptidase homologues (MER047504), subfamily A2A non-peptidase homologues (MER047511), subfamily A2A non-peptidase homologues (MER047513), subfamily A2A non-peptidase homologues (MER047514), subfamily A2A non-peptidase homologues (MER047515), subfamily A2A non-peptidase homologues (MER047516), subfamily A2A non-peptidase homologues (MER047520), subfamily A2A non-peptidase homologues (MER047533), subfamily A2A non-peptidase homologues (MER047537), subfamily A2A non-peptidase homologues (MER047569), subfamily A2A non-peptidase homologues (MER047570), subfamily A2A non-peptidase homologues (MER047584), subfamily A2A non-peptidase homologues (MER047603), subfamily A2A non-peptidase homologues (MER047604), subfamily A2A non-peptidase homologues (MER047606), subfamily A2A non-peptidase homologues (MER047609), subfamily A2A non-peptidase homologues (MER047616), subfamily A2A non-peptidase homologues (MER047619), subfamily A2A non-peptidase homologues (MER047648), subfamily A2A non-peptidase homologues (MER047649), subfamily A2A non-peptidase homologues (MER047662), subfamily A2A non-peptidase homologues (MER048004), subfamily A2A non-peptidase homologues (MER048018), subfamily A2A non-peptidase homologues (MER048019), subfamily A2A non-peptidase homologues (MER048023), subfamily A2A non-peptidase homologues (MER048037), subfamily A2A unassigned peptidases (MER047164), subfamily A2A unassigned peptidases (MER047231), subfamily A2A unassigned peptidases (MER047386), skin aspartic protease (MER057097), presenilin 1 (MER005221), presenilin 2

(MER005223), impas 1 peptidase (MER019701), impas 1 peptidase (MER184722), impas 4 peptidase (MER019715), impas 2 peptidase (MERO 19708), impas 5 peptidase (MER019712), impas 3 peptidase (MER019711), possible family A22 pseudogene (Homo sapiens chromosome 18) (MER029974), possible family A22 pseudogene (Homo sapiens chromosome 11) (MER023159), cathepsin V (MER004437), cathepsin X (MER004508), cathepsin F (MER004980), cathepsin L (MER000622), cathepsin S (MER000633), cathepsin O (MER001690), cathepsin K (MER000644), cathepsin W (MER003756), cathepsin H (MER000629), cathepsin B (MER000686), dipeptidyl-peptidase I (MER001937), bleomycin hydrolase (animal) (MER002481), tubulointerstitial nephritis antigen (MER016137), tubulointerstitial nephritis antigen-related protein (MER021799), cathepsin L-like pseudogene 1 (Homo sapiens) (MER002789), cathepsin B-like pseudogene (chromosome 4, Homo sapiens) (MER029469), cathepsin B-like pseudogene (chromosome 1, Homo sapiens) (MER029457), CTSLL2 g.p. (Homo sapiens) (MER005210), CTSLL3 g.p. (Homo sapiens) (MER005209), calpain-1 (MER000770), calpain-2 (MER000964), calpain-3 (MER001446), calpain-9 (MER004042), calpain-8 (MER021474), calpain-l 5 (MER004745), calpain-5 (MER002939), calpain-l 1 (MER005844), calpain-12 (MER029889), calpain-10 (MER013510), calpain-13 (MER020139), calpain-14 (MER029744), Mername-AA253 peptidase (MER005537), calpamodulin (MER000718), hypothetical protein flj40251 (MER003201), ubiquitinyl hydrolase-L1 (MER000832), ubiquitinyl hydrolase-L3 (MER000836), ubiquitinyl hydrolase-BAPI (MER003989), ubiquitinyl hydrolase-UCH37 (MER005539), ubiquitin-specific peptidase 5 (MER002066), ubiquitin-specific peptidase 6 (MER000863), ubiquitin-specific peptidase 4 (MEROO 1795), ubiquitin-specific peptidase 8
(MER001884), ubiquitin-specific peptidase 13 (MER002627), ubiquitin-specific peptidase 2 (MER004834), ubiquitin-specific peptidase 11 (MER002693), ubiquitin-specific peptidase 14 (MER002667), ubiquitin-specific peptidase 7 (MER002896), ubiquitin-specific peptidase 9× (MER005877), ubiquitin-specific peptidase 10 (MER004439), ubiquitin-specific peptidase 1 (MER004978), ubiquitin-specific peptidase 12 (MER005454), ubiquitin-specific peptidase 16 (MER005493), ubiquitin-specific peptidase 15 (MER005427), ubiquitin-specific peptidase 17 (MER002900), ubiquitin-specific peptidase 19 (MER005428), ubiquitin-specific peptidase 20 (MER005494), ubiquitin-specific peptidase 3 (MER005513), ubiquitin-specific peptidase 9Y (MER004314), ubiquitin-specific peptidase 18 (MER005641), ubiquitin-specific peptidase 21 (MER006258), ubiquitin-specific peptidase 22 (MER012130), ubiquitin-specific peptidase 33 (MER014335), ubiquitin-specific peptidase 29 (MER012093), ubiquitin-specific peptidase 25 (MER011115), ubiquitin-specific peptidase 36 (MER014033), ubiquitin-specific peptidase 32 (MER014290), ubiquitin-specific peptidase 26 (Homo sapiens-type) (MERO 14292), ubiquitin-specific peptidase 24 (MER005706), ubiquitin-specific peptidase 42 (MER011852), ubiquitin-specific peptidase 46 (MER014629), ubiquitin-specific peptidase 37 (MER014633), ubiquitin-specific peptidase 28 (MER014634), ubiquitin-specific peptidase 47 (MERO 14636), ubiquitin-specific peptidase 38 (MERO 14637), ubiquitin-specific peptidase 44 (MER014638), ubiquitin-specific peptidase 50 (MER030315), ubiquitin-specific peptidase 35 (MERO 14646), ubiquitin-specific peptidase 30 (MERO 14649), Mername-AA091 peptidase (MER014743), ubiquitin-specific peptidase 45 (MER030314), ubiquitin-specific peptidase 51 (MER014769), ubiquitin-specific peptidase 34 (MER014780), ubiquitin-specific peptidase 48 (MER064620), ubiquitin-specific peptidase 40 (MERO 15483), ubiquitin-specific peptidase 41 (MER045268), ubiquitin-specific peptidase 31 (MER015493), Mername-AA129 peptidase (MER016485), ubiquitin-specific peptidase 49 (MER016486), Mername-AA187 peptidase (MER052579), ETSP17-like peptidase (MER030192), ubiquitin-specific peptidase 54 (MER028714), ubiquitin-specific peptidase 53 (MER027329), ubiquitin-specific endopeptidase 39 [misleading] (MER064621), Memame-AA090 non-peptidase homologue (MERO 14739), ubiquitin-specific peptidase [misleading] (MER030140), ubiquitin-specific peptidase 52 [misleading] (MER030317), NEK2 pseudogene (MER014736), C19 pseudogene (Homo sapiens: chromosome 5) (MER029972), Memame-AA088 peptidase (MER014750), autophagin-2 (MER013564), autophagin-1 (MER013561), autophagin-3 (MER014316), autophagin-4 (MER064622), Cezanne deubiquitinylating peptidase (MER029042), Cezanne-2 peptidase (MER029044), tumor necrosis factor alpha-induced protein 3 (MER029050), trabid peptidase (MER029052), VCIP135 deubiquitinating peptidase (MER152304), otubain-1 (MER029056), otubain-2 (MER029061), CylD protein (MER030104), UfSP1 peptidase (MER042724), ETfSP2 peptidase (MER060306), DEIBA deubiquitinylating enzyme (MER086098), KIAA0459 (Homo sapiens)-like protein (MER122467), Otudl protein (MER125457), glycosyltransferase 28 domain containing 1, isoform CRA c (Homo sapiens)-like (MER123606), hinIL g.p. (Homo sapiens) (MER139816), ataxin-3 (MER099998), ATXN3L putative peptidase (MER115261), Josephin domain containing 1 (Homo sapiens) (MER125334), Josephin domain containing 2 (Homo sapiens) (MER124068), YOD1 peptidase (MER116559), legumain (plant alpha form) (MER044591), legumain (MER001800), glycosylphosphatidylinositokprotein transamidase (MER002479), legumain pseudogene (Homo sapiens) (MER029741), family C13 unassigned peptidases (MER175813), caspase-1 (MER000850), caspase-3 (MER000853), caspase-7 (MER002705), caspase-6 (MER002708), caspase-2 (MER001644), caspase-4 (MER001938), caspase-5 (MER002240), caspase-8 (MER002849), caspase-9 (MER002707), caspase-10 (MER002579), caspase-14 (MER012083), paracaspase (MER019325), Memame-AA143 peptidase (MER021304), Mername-AA186 peptidase (MER020516), putative caspase (Homo sapiens) (MER021463), FLIP protein (MER003026), Memame-AA142 protein (MER021316), caspase-12 pseudogene (Homo sapiens) (MER019698), Mername-AA093 caspase pseudogene (MER014766), subfamily C14A non-peptidase homologues (MER185329), subfamily C14A non-peptidase homologues (MER179956), separase (Homo sapiens-type) (MER011775), separase-like pseudogene (MER014797), SENP1 peptidase (MER011012), SENP3 peptidase (MER011019), SENP6 peptidase (MER011109), SENP2 peptidase (MER012183), SENP5 peptidase (MER014032), SENP7 peptidase (MER014095), SENP8 peptidase (MER016161), SENP4 peptidase (MER005557), pyroglutamyl-peptidase I (chordate) (MER011032), Memame-AA073 peptidase (MER029978), Sonic hedgehog protein (MER002539), Indian hedgehog protein (MER002538), Desert hedgehog protein (MER012170), dipeptidyl-peptidase III (MER004252), Mername-AA164 protein (MER020410), LOC138971 g.p. (Homo sapiens) (MER020074), Atp23 peptidase (MER060642), prenyl peptidase 1 (MER004246), aminopeptidase N (MER000997), aminopeptidase A (MER001012), leukotriene A4 hydrolase (MER001013), pyroglutamyl-peptidase II (MER012221), cytosol alanyl aminopeptidase (MER002746), cystinyl aminopeptidase (MER002060), aminopeptidase B (MER001494), aminopeptidase PILS (MER005331), arginyl aminopeptidase-like 1 (MERO 12271), leukocyte-derived arginine aminopeptidase (MER002968), aminopeptidase Q (MER052595), aminopeptidase 0 (MER019730), Tata binding protein associated factor (MER026493), angiotensin-converting enzyme peptidase unit 1 (MER004967), angiotensin-converting enzyme peptidase unit 2 (MER001019), angiotensin-converting enzyme-2 (MER011061), Memame-AA153 protein (MER020514), thimet oligopeptidase (MER001737), neurolysin (MERO 10991), mitochondrial intermediate peptidase (MER003665), Mername-AA154 protein (MER021317), leishmanolysin-2 (MER014492), leishmanolysin-3 (MER180031), matrix metallopeptidase-1 (MER001063), matrix metallopeptidase-8 (MER001084), matrix metallopeptidase-2 (MER001080), matrix metallopeptidase-9 (MER001085), matrix metallopeptidase-3 (MER001068), matrix metallopeptidase-10 (Homo sapiens-type) (MER001072), matrix metallopeptidase-l 1 (MER001075), matrix metallopeptidase-7 (MER001092), matrix metallopeptidase-l 2 (MER001089), matrix metallopeptidase-l 3 (MER001411), membrane-type matrix metallopeptidase-l (MER001077), membrane-type matrix metallopeptidase-2 (MER002383), membrane-type matrix metallopeptidase-3 (MER002384), membrane-type matrix metallopeptidase-4 (MER002595), matrix metallopeptidase-20 (MER003021), matrix metallopeptidase-l 9 (MER002076), matrix metallopeptidase-23B (MER004766), membrane-type matrix metallopeptidase-5 (MER005638), membrane-type matrix metallopeptidase-6 (MERO 12071), matrix metallopeptidase-21 (MER006101), matrix metallopeptidase-22 (MERO 14098), matrix metallopeptidase-26 (MERO 12072), matrix metallopeptidase-28 (MER013587), matrix metallopeptidase-23A (MER037217), macrophage elastase homologue (chromosome 8, Homo sapiens) (MER030035), Memame-AA156 protein (MER021309), matrix metallopeptidase-like 1 (MER045280), subfamily M10A non-peptidase homologues (MER175912), subfamily M10A non-peptidase homologues (MER187997), subfamily M10A non-peptidase homologues (MER187998), subfamily M10A non-peptidase homologues (MER180000), meprin alpha subunit (MER001111), meprin beta subunit (MER005213), procollagen C-peptidase (MER001113), mammalian tolloid-like 1 protein (MER005124), mammalian-type tolloid-like 2 protein (MER005866), ADAMTS9 peptidase (MER012092), ADAMTS14 peptidase (MER016700), ADAMTS15 peptidase (MERO 17029), ADAMTS16 peptidase (MER015689), ADAMTS17 peptidase (MERO 16302), ADAMTS18 peptidase (MERO 16090), ADAMTS19 peptidase (MERO 15663), ADAMS peptidase (MER003902), ADAM9 peptidase (MER001140), ADAM 10 peptidase (MER002382), ADAM 12 peptidase (MER005107), ADAM 19 peptidase (MERO 12241), ADAM 15 peptidase (MER002386), ADAM 17 peptidase (MER003094), ADAM20 peptidase (MER004725), ADAMDEC1 peptidase (MER000743), ADAMTS3 peptidase (MER005100), ADAMTS4 peptidase (MER005101), ADAMTS1 peptidase (MER005546), ADAM28 peptidase (Homo sapiens-type) (MER005495), ADAMTS5 peptidase (MER005548), ADAMTS8 peptidase (MER005545), ADAMTS6 peptidase (MER005893), ADAMTS7 peptidase (MER005894), ADAM30 peptidase (MER006268), ADAM21 peptidase (Homo sapiens-type) (MER004726), ADAMTS10 peptidase (MER014331), AD AMTS 12 peptidase (MER014337), ADAMTS13 peptidase (MER015450), ADAM33 peptidase (MER015143), ovastacin (MER029996), ADAMTS20 peptidase (Homo sapiens-type) (MER026906), procollagen I N-peptidase (MER004985), ADAM2 protein (MER003090), ADAM6 protein (MER047044), ADAM7 protein (MER005109), ADAM18 protein (MER012230), ADAM32 protein (MER026938), non-peptidase homologue (Homo sapiens chromosome 4) (MER029973), family M12 non-peptidase homologue (Homo sapiens chromosome 16) (MER047654), family M12 non-peptidase homologue (Homo sapiens chromosome 15) (MER047250), ADAM3B protein (Homo sapiens-type) (MER005199), ADAM11 protein (MER001146), ADAM22 protein (MER005102), ADAM23 protein (MER005103), ADAM29 protein (MER006267), protein similar to ADAM21 peptidase preproprotein (Homo sapiens) (MER026944), Memame-AA225 peptidase homologue (Homo sapiens) (MER047474), putative ADAM pseudogene (chromosome 4, Homo sapiens) (MER029975), ADAM3A g.p. (Homo sapiens) (MER005200), ADAM1 g.p. (Homo sapiens) (MER003912), subfamily M12B non peptidase homologues (MER188210), subfamily M12B non-peptidase homologues (MER188211), subfamily M12B non-peptidase homologues (MER188212), subfamily M12B non-peptidase homologues (MER188220), neprilysin (MER001050), endothelin-converting enzyme 1 (MEROO 1057), endothelin-converting enzyme 2 (MER004776), DINE peptidase (MER005197), neprilysin-2 (MER013406), Kell blood-group protein (MEROO 1054), PHEX peptidase (MER002062), i-AAA peptidase (MEROO 1246), i-AAA peptidase (MER005755), paraplegin (MER004454), Afg3-like protein 2 (MER005496), Afg3-like protein 1A (MER014306), pappalysin-1 (MER002217), pappaly sin-2 (MER014521), famesylated-protein converting enzyme 1 (MER002646), metalloprotease-related protein-1 (MER030873), aminopeptidase AMZ2 (MER011907), aminopeptidase AMZ1 (MER058242), carboxypeptidase A1 (MER001190), carboxypeptidase A2 (MEROO 1608), carboxypeptidase B (MEROO 1194), carboxypeptidase N (MEROO 1198), carboxypeptidase E (MEROO 1199), carboxypeptidase M (MEROO 1205), carboxypeptidase U (MEROO 1193), carboxypeptidase A3 (MEROO 1187), metallocarboxypeptidase D peptidase unit 1 (MER003781), metallocarboxypeptidase Z (MER003428), metallocarboxypeptidase D peptidase unit 2 (MER004963), carboxypeptidase A4 (MER013421), carboxypeptidase A6 (MER013456), carboxypeptidase A5 (MER017121), metallocarboxypeptidase 0 (MER016044), cytosolic carboxypeptidase-like protein 5 (MER033174), cytosolic carboxypeptidase 3 (MER033176), cytosolic carboxypeptidase 6 (MER033178), cytosolic carboxypeptidase 1 (MER033179), cytosolic carboxypeptidase 2 (MER037713), metallocarboxypeptidase D non-peptidase unit (MER004964), adipocyte-enhancer binding protein 1 (MER003889), carboxypeptidase-like protein XI (MER013404), carboxypeptidase-like protein X2 (MER078764), cytosolic carboxypeptidase (MER026952), family M14 non-peptidase homologues (MER199530), insulysin (MER001214), mitochondrial processing peptidase beta-subunit (MER004497), nardilysin (MER003883), eupitrilysin (MER004877), mitochondrial processing peptidase non-peptidase alpha subunit (MER001413), ubiquinol-cytochrome c reductase core protein I (MER003543), ubiquinol-cytochrome c reductase core protein II (MER003544), ubiquinol-cytochrome c reductase core protein domain 2 (MER043998), insulysin unit 2 (MER046821), nardilysin unit 2 (MER046874), insulysin unit 3 (MER078753), mitochondrial processing peptidase subunit alpha unit 2 (MER124489), nardilysin unit 3 (MER142856), LOC133083 g.p. (Homo sapiens) (MER021876), subfamily M16B non-peptidase homologues (MER188757), leucyl aminopeptidase (animal) (MER003100), Mername-AA040 peptidase (MER003919), leucyl aminopeptidase-1 (Caenorhabditis-type) (MER013416), methionyl aminopeptidase 1 (MEROO 1342), methionyl aminopeptidase 2 (MER001728), aminopeptidase P2 (MER004498), Xaa-Pro dipeptidase (eukaryote) (MEROO 1248), aminopeptidase P1 (MER004321), mitochondrial intermediate cleaving peptidase 55 kDa (MER013463), mitochondrial methionyl aminopeptidase (MER014055), Mername-AA020 peptidase homologue (MERO 10972), proliferation-association protein 1 (MER005497), chromatin-specific transcription elongation factor 140 kDa subunit (MER026495), proliferation-associated protein 1-like (Homo sapiens chromosome X) (MER029983), Mername-AA226 peptidase homologue (Homo sapiens) (MER056262), Mername-AA227 peptidase homologue (Homo sapiens) (MER047299), subfamily M24A non-peptidase homologues (MER179893), aspartyl aminopeptidase (MER003373), Gly-Xaa carboxypeptidase (MER033182), carnosine dipeptidase II (MERO 14551), carnosine dipeptidase I (MER015142), Memame-AA161 protein (MER021873), aminoacylase (MER001271), glutamate carboxypeptidase II (MER002104), NAALADASE L peptidase (MER005239), glutamate carboxypeptidase III (MER005238), plasma glutamate carboxypeptidase (MER005244), Mername-AA103 peptidase (MER015091), Fxna peptidase (MER029965), transferrin receptor protein (MER002105), transferrin receptor 2 protein (MER005152), glutaminyl cyclise (MERO 15095), glutamate carboxypeptidase II (Homo sapiens)-type non peptidase homologue (MER026971), nicalin (MER044627), membrane dipeptidase (MER001260), membrane-bound dipeptidase-2 (MER013499), membrane-bound dipeptidase-3 (MERO 13496), dihydro-orotase (MER005767), dihydropyrimidinase (MER033266), dihydropyrimidinase related protein-1 (MER030143), dihydropyrimidinase related protein-2 (MER030155), dihydropyrimidinase related protein-3 (MER030151), dihydropyrimidinase related protein-4 (MER030149), dihydropyrimidinase related protein-5 (MER030136), hypothetical protein like 5730457F11RIK (MER033184), 1300019j08rik protein (MER033186)), guanine aminohydrolase (MER037714), Kael putative peptidase (MEROO 1577), OSGEPL1-like protein (MER013498), S2P peptidase (MER004458), subfamily M23B non-peptidase homologues (MER199845), subfamily M23B non-peptidase homologues (MER199846), subfamily M23B non-peptidase homologues (MER199847), subfamily M23B non-peptidase homologues (MER137320), subfamily M23B non-peptidase homologues (MER201557), subfamily M23B non-peptidase homologues (MER199417), subfamily M23B non-peptidase homologues (MER199418), subfamily M23B non-peptidase homologues (MER199419), subfamily M23B non-peptidase homologues (MER199420), subfamily M23B non-peptidase homologues (MER175932), subfamily M23B non-peptidase homologues (MER199665), Pohl peptidase (MER020382), Jabl/MPN domain metalloenzyme (MER022057), Mername-AA165 peptidase (MER021865), Brcc36 isopeptidase (MER021890), histone H2A deubiquitinase MYSM1 (MER021887), AMSH deubiquitinating peptidase (MER030146), putative peptidase (Homo sapiens chromosome 2) (MER029970), Memame-AA168 protein (MER021886), COP9 signalosome subunit 6 (MER030137), 26S proteasome non-ATPase regulatory subunit 7 (MER030134), eukaryotic translation initiation factor 3 subunit 5 (MER030133), 1FP38 peptidase homologue (MER030132), subfamily M67A non-peptidase homologues (MER191181), subfamily M67A unassigned peptidases (MER191144), granzyme B (Homo sapiens-type) (MER000168), testisin (MER005212), tryptase beta (MER000136), kallikrein-related peptidase 5 (MER005544), conn (MER005881), kallikrein-related peptidase 12 (MER006038), DESC1 peptidase (MER006298), tryptase gamma 1 (MER011036), kallikrein-related peptidase 14 (MER011038), hyaluronan-binding peptidase (MER003612), transmembrane peptidase, serine 4 (MER011104), intestinal serine peptidase (rodent) (MER016130), adrenal secretory serine peptidase (MER003734), tryptase delta 1 (Homo sapiens) (MER005948), matriptase-3 (MER029902), marapsin (MER006119), tryptase-6 (MER006118), ovochymase-1 domain 1 (MER099182), transmembrane peptidase, serine 3 (MER005926), kallikrein-related peptidase 15 (MER000064), Mername-AA031 peptidase (MER014054), DMPRSS13 peptidase (MER014226), Mername-AA038 peptidase (MER062848), Mername-AA204 peptidase (MER029980), cationic trypsin (Homo sapiens-type) (MER000020), elastase-2 (MER000118), mannan-binding lectin-associated serine peptidase-3 (MER031968), cathepsin G (MER000082), myeloblastin (MER000170), granzyme A (MER001379), granzyme M (MER001541), chymase (Homo sapiens-type) (MER000123), tryptase alpha (MER000135), granzyme K (MER001936), granzyme H (MER000166), chymotrypsin B (MER000001), elastase-1 (MER003733), pancreatic endopeptidase E (MER000149), pancreatic elastase II (MER000146), enteropeptidase (MER002068), chymotrypsin C (MER000761), prostasin (MER002460), kallikrein 1 (MER000093), kallikrein-related peptidase 2 (MER000094), kallikrein-related peptidase 3 (MER000115), mesotrypsin (MER000022), complement component Clr-like peptidase (MER016352), complement factor D (MER000130), complement component activated Clr (MER000238), complement component activated Cls (MER000239), complement component C2a (MER000231), complement factor B (MER000229), mannan-binding lectin-associated serine peptidase 1 (MER000244), complement factor I (MER000228), pancreatic endopeptidase E form B (MER000150), pancreatic elastase IIB (MER000147), coagulation factor Xlla (MER000187), plasma kallikrein (MER000203) coagulation factor Xia (MER000210), coagulation factor IXa (MER000216), coagulation factor Vila (MER000215), coagulation factor Xa (MER000212), thrombin (MER000188), protein C (activated) (MER000222), acrosin (MER000078), hepsin (MER000156), hepatocyte growth factor activator (MER000186), mannan-binding lectin-associated serine peptidase 2 (MER002758), u-plasminogen activator (MER000195), t-plasminogen activator
(MER000192), plasmin (MER000175), kallikrein-related peptidase 6 (MER002580), neurotrypsin (MER004171), kallikrein-related peptidase 8 (MER005400), kallikrein-related peptidase 10 (MER003645), epitheliasin (MER003736), kallikrein-related peptidase 4 (MER005266), prosemin (MER004214), chymopasin (MER001503), kallikrein-related peptidase 11 (MER004861), kallikrein-related peptidase 11 (MER216142), trypsin-2 type A (MER000021), HtrA1 peptidase (Homo sapiens-type) (MER002577), HtrA2 peptidase (MER208413), HtrA2 peptidase (MER004093), HtrA3 peptidase (MER014795), HtrA4 peptidase (MER016351), Tysndl peptidase (MER050461), DMPRSS12 peptidase (MER017085), HAT-like putative peptidase 2 (MER021884), trypsin C (MER021898), kallikrein-related peptidase 7 (MER002001), matriptase (MER003735), kallikrein-related peptidase 13 (MER005269), kallikrein-related peptidase 9 (MER005270), matriptase-2 (MER005278), umbelical vein peptidase (MER005421), LCLP peptidase (MER001900), spinesin (MER014385), marapsin-2 (MER021929), complement factor D-like putative peptidase (MER056164), ovochymase-2 (MER022410), HAT-like 4 peptidase (MER044589), ovochymase 1 domain 1 (MER022412), epidermis-specific SP-like putative peptidase (MER029900), testis serine peptidase 5 (MER029901), Mername-AA258 peptidase (MER000285), polyserase-IA unit 1 (MER030879), polyserase-IA unit 2 (MER030880), testis serine peptidase 2 (human-type) (MER033187), hypothetical acrosin-like peptidase (Homo sapiens) (MER033253), HAT-like 5 peptidase (MER028215), polyserase-3 unit 1 (MER061763), polyserase-3 unit 2 (MER061748), peptidase similar to tryptophan/serine protease (MER056263), polyserase-2 unit 1 (MER061777), Memame-AA123 peptidase (MER021930), HAT-like 2 peptidase (MER099184), hCG2041 452-like protein (MER099172), hCG22067 (Homo sapiens) (MER099169), brain-rescue-factor-1 (Homo sapiens) (MER098873), hCG204H08 (Homo sapiens) (MER099173), polyserase-2 unit 2 (MER061760), polyserase-2 unit 3 (MER065694), Mername-AA201 (peptidase homologue) MER099175, secreted trypsin-like serine peptidase homologue (MER030000), polyserase-1A unit 3 (MER029880), azurocidin (MER000119), haptoglobin-1 (MER000233), haptoglobin-related protein (MER000235), macrophage-stimulating protein (MER001546), hepatocyte growth factor (MER000185), protein Z (MER000227), TESP1 protein (MER047214), LOC136242 protein (MER016132), plasma kallikrein-like protein 4 (MERO 16346), PRSS35 protein (MER016350), DKFZp586H2123-like protein (MER066474), apolipoprotein (MER000183), psi-KLK1 pseudogene (Homo sapiens) (MER033287), tryptase pseudogene I (MER015077), tryptase pseudogene II (MER015078), tryptase pseudogene III (MER015079), subfamily S1A unassigned peptidases (MER216982), subfamily S1A unassigned peptidases (MER216148), amidophosphoribosyltransferase precursor (MER003314), glutamine-fructose-6-phosphate transaminase 1 (MER003322), glutamine: fructose-6-phosphate amidotransferase (MER012158), Mername-AA144 protein (MER021319), asparagine synthetase (MER033254), family C44 non-peptidase homologues (MER159286), family C44 unassigned peptidases (MER185625) family C44 unassigned peptidases (MER185626), secernin 1 (MER045376), secernin 2 (MER064573), secernin 3 (MER064582), acid ceramidase precursor (MER100794), N-acylethanolamine acid amidase precursor (MER141667), proteasome catalytic subunit 1 (MER000556), proteasome catalytic subunit 2 (MER002625), proteasome catalytic subunit 3 (MER002149), proteasome catalytic subunit 1i (MER000552), proteasome catalytic subunit 2i (MER001515), proteasome catalytic subunit 3i (MER000555), proteasome catalytic subunit 5t (MER026203), protein serine kinase c17 (MER026497), proteasome subunit alpha 6 (MER000557), proteasome subunit alpha 2 (MER000550), proteasome subunit alpha 4 (MER000554), proteasome subunit alpha 7 (MER033250), proteasome subunit alpha 5 (MER000558), proteasome subunit alpha 1 (MER000549), proteasome subunit alpha 3 (MER000553), proteasome subunit XAPC7 (MER004372), proteasome subunit beta 3 (MER001710), proteasome subunit beta 2 (MER002676), proteasome subunit beta 1 (MER000551), proteasome subunit beta 4 (MER001711), Mername-AA230 peptidase homologue (Homo sapiens) (MER047329), Memame-AA23 l pseudogene (Homo sapiens) (MER047172), Mername-AA232 pseudogene (Homo sapiens) (MER047316), glycosylasparaginase precursor (MER003299), isoaspartyl dipeptidase (threonine type) (MER031622), taspase-1 (MERO 16969), gamma-glutamyltransferase 5 (mammalian-type) (MEROO 1977), gamma-glutamyltransferase 1 (mammalian-type) (MEROO 1629), gamma-glutamyltransferase 2 (Homo sapiens) (MER001976), gamma-glutamyltransferase-like protein 4 (MER002721), gamma-glutamyltransferase-like protein 3 (MERO 16970), similar to gamma-glutamyltransferase 1 precursor (Homo sapiens) (MER026204), similar to gamma-glutamyltransferase 1 precursor (Homo sapiens) (MER026205), Memame-AA21 l putative peptidase (MER026207), gamma-glutamyltransferase 6 (MER 159283), gamma-glutamyl transpeptidase homologue (chromosome 2, Homo sapiens) (MER037241), polycystin-l (MER126824), KIAA1879 protein (MER159329), polycystic kidney disease 1-like 3 (MER172554), gamma-glutamyl hydrolase (MER002963), guanine 5″-monophosphate synthetase (MER043387), carbamoyl-phosphate synthase (Homo sapiens-type) (MER078640), dihydro-orotase (N-terminal unit) (Homo sapiens-type) (MER060647) DJ-1 putative peptidase (MER003390), Mername-AA100 putative peptidase (MER014802), Memame-AAlOl non-peptidase homologue (MER014803), KIAA0361 protein (Homo sapiens-type) (MER042827), Fl 134283 protein (Homo sapiens) (MER044553), non-peptidase homologue chromosome 21 open reading frame 33 (Homo sapiens) (MER160094), family C56 non-peptidase homologues (MER177016), family C56 non-peptidase homologues (MER176613), family C56 non peptidase homologues (MER176918), EGF-like module containing mucin-like hormone receptor-like 2 (MER037230), CD97 antigen (human type) (MER037286), EGF-like module containing mucin-like hormone receptor-like 3 (MER037288), EGF-like module containing mucin-like hormone receptor-like 1 (MER037278), EGF-like module containing mucin-like hormone receptor-like 4 (MER037294), cadherin EGF LAG seven-pass G-type receptor 2 precursor (Homo sapiens) (MER045397), Gpr64 (Mus musculus)-type protein (MER123205), GPR56 (Homo sapiens)-type protein (MER122057), latrophilin 2 (MER122199), latrophilin-1 (MER126380), latrophilin 3 (MER124612), protocadherin Flamingo 2 (MER124239), ETL protein (MER126267), G protein-coupled receptor 112 (MER126114), seven transmembrane helix receptor (MER125448), Gprl 14 protein (MER159320), GPR126 vascular inducible G protein-coupled receptor (MER140015), GPR125 (Homo sapiens)-type protein (MER159279), GPR116 (Homo sapiens)-type G-protein coupled receptor (MER159280), GPR128 (Homo sapiens)-type G-protein coupled receptor (MER162015), GPR133 (Homo sapiens)-type protein (MER159334), GPR110 G-protein coupled receptor (MER159277), GPR97 protein (MER159322), KPG 006 protein (MER161773), KPG 008 protein (MER161835), KPG 009 protein (MER159335), unassigned homologue (MER166269), GPR113 protein (MER159352), brain-specific angiogenesis inhibitor 2 (MER159746), PIDD auto-processing protein unit 1 (MER020001), PIDD auto-processing protein unit 2 (MER063690), MUCI self-cleaving mucin (MER074260), dystroglycan (MER054741), proprotein convertase 9 (MER022416), site-1 peptidase (MER001948), furin (MER000375), proprotein convertase 1 (MER000376), proprotein convertase 2 (MER000377), proprotein convertase 4 (MER028255), PACE4 proprotein convertase (MER000383), proprotein convertase 5 (MER002578), proprotein convertase 7 (MER002984), tripeptidyl-peptidase II (MER000355), subfamily S8A non peptidase homologues (MER201339), subfamily S8A non-peptidase homologues (MER191613), subfamily S8A unassigned peptidases (MER191611), subfamily S8A unassigned peptidases (MER191612), subfamily S8A unassigned peptidases (MER191614), tripeptidyl-peptidase I (MER003575), prolyl oligopeptidase (MER000393), dipeptidyl-peptidase IV (eukaryote) (MER000401), acylaminoacyl-peptidase (MER000408), fibroblast activation protein alpha subunit (MER000399), PREPL A protein (MER004227), dipeptidyl-peptidase 8 (MER013484), dipeptidyl-peptidase 9 (MER004923), FLJ1 putative peptidase (MER017240), Mername-AA194 putative peptidase (MER017353), Mername-AA195 putative peptidase (MER017367), Memame-AA196 putative peptidase (MER017368), Memame-AA197 putative peptidase (MER017371), C14orf29 protein (MER033244), hypothetical protein (MER033245), hypothetical esterase/lipase/thioesterase (MER047309), protein bat5 (MER037840), hypothetical protein flj40219 (MER033212), hypothetical protein flj 37464 (MER033240), hypothetical protein flj33678 (MER033241), dipeptidylpeptidase homologue DPP6 (MER000403), dipeptidylpeptidase homologue DPP 10 (MER005988), protein similar to Mus musculus chromosome 20 open reading frame 135 (MER037845), kynurenine formamidase (MER046020), thyroglobulin precursor (MER011604), acetylcholinesterase (MER033188), cholinesterase (MER033198), carboxylesterase DI (MER033213), liver carboxylesterase (MER033220), carboxylesterase 3 (MER033224), carboxylesterase 2 (MER033226), bile salt-dependent lipase (MER033227), carboxylesterase-related protein (MER033231), neuroligin 3 (MER033232), neuroligin 4, X-linked (MER033235), neuroligin 4, Y-linked (MER033236), esterase D (MER043126), arylacetamide deacetylase (MER033237), KIAA1363-like protein (MER033242), hormone-sensitive lipase (MER033274), neuroligin 1 (MER033280), neuroligin 2 (MER033283), family S9 non-peptidase homologues (MER212939), family S9 non-peptidase homologues (MER211490), subfamily S9C unassigned peptidases (MER192341), family S9 unassigned peptidases (MER209181), family S9 unassigned peptidases (MER200434), family S9 unassigned peptidases (MER209507), family S9 unassigned peptidases (MER209142), serine carboxypeptidase A (MER000430), vitellogenic carboxypeptidase-like protein (MER005492), RISC peptidase (MERO 10960), family S15 unassigned peptidases (MER199442), family S15 unassigned peptidases (MER200437), family S15 unassigned peptidases (MER212825), lysosomal Pro-Xaa carboxypeptidase (MER000446), dipeptidyl-peptidase II (MER004952), thymus-specific serine peptidase (MER005538), epoxide hydrolase-like putative peptidase (MER031614), Loc3285744ike protein (MER033246), abhydrolase domain-containing protein 4 (MER031616), epoxide hydrolase (MER000432), mesoderm specific transcript protein (MER199890), mesoderm specific transcript protein (MER017123), cytosolic epoxide hydrolase (MER029997), cytosolic epoxide hydrolase (MER213866), similar to hypothetical protein FLJ22408 (MER031608), CGI-58 putative peptidase (MER030163), Williams-Beuren syndrome critical region protein 21 epoxide hydrolase (MER031610), epoxide hydrolase (MER031612), hypothetical protein flj22408 (epoxide hydrolase) (MER031617), monoglyceride lipase (MER033247), hypothetical protein (MER033249), valacyclovir hydrolase (MER033259), Ccgl-interacting factor b (MER210738), glycosylasparaginase precursor (MER003299), isoaspartyl dipeptidase (threonine type) (MER031622), taspase-1 (MER016969), gamma-glutamyltransferase 5 (mammalian-type) (MER001977), gamma-glutamyltransferase 1 (mammalian-type) (MER001629), gamma-glutamyltransferase 2 (Homo sapiens) (MER001976), gamma-glutamyltransferase-like protein 4 (MER002721), gamma-glutamyltransferase-like protein 3 (MERO 16970), similar to gamma-glutamyltransferase 1 precursor (Homo sapiens) (MER026204), similar to gamma-glutamyltransferase 1 precursor (Homo sapiens) (MER026205), Mername-AA21 l putative peptidase (MER026207), gamma-glutamyltransferase 6 (MER159283), gamma-glutamyl transpeptidase homologue (chromosome 2, Homo sapiens) (MER037241), polycystin-1 (MER126824), KIAA1879 protein (MER 159329), polycystic kidney disease 1-like 3 (MER172554), gamma-glutamyl hydrolase (MER002963), guanine 5″-monophosphate synthetase (MER043387), carbamoyl-phosphate synthase (Homo sapiens-type) (MER078640), dihydro-orotase (N-terminal unit) (Homo sapiens-type) (MER060647), DJ-1 putative peptidase (MER003390), Memame-AAlOO putative peptidase (MER014802), Memame-AAlOl non-peptidase homologue (MER014803), KIAA0361 protein (Homo sapiens-type) (MER042827), FLJ34283 protein (Homo sapiens) (MER044553), non-peptidase homologue chromosome 21 open reading frame 33 (Homo sapiens) (MER160094), family C56 non-peptidase homologues (MER177016), family C56 non-peptidase homologues (MER176613), family C56 non-peptidase homologues (MER176918), EGF-like module containing mucin-like hormone receptor-like 2 (MER037230), CD97 antigen (human type) (MER037286), EGF-like module containing mucin-like hormone receptor-like 3 (MER037288), EGF-like module containing mucin-like hormone receptor-like 1 (MER037278), EGF-like module containing mucin-like hormone receptor-like 4 (MER037294), cadherin EGF LAG seven-pass G-type receptor 2 precursor (Homo sapiens) (MER045397), Gpr64 (Mus musculus)-type protein (MER123205), GPR56 (Homo sapiens)-type protein (MER122057), latrophilin 2 (MER122199), latrophilin-1 (MER126380), latrophilin 3 (MER124612), protocadherin Flamingo 2 (MER 124239), ETL protein (MER126267), G protein-coupled receptor 112 (MER126114), seven transmembrane helix receptor (MER125448), Gprl 14 protein (MER159320), GPR126 vascular inducible G protein-coupled receptor (MER140015), GPR125 (Homo sapiens)-type protein (MER159279), GPR116 (Homo sapiens)-type G-protein coupled receptor (MER159280), GPR128 (Homo sapiens)-type G-protein coupled receptor (MER162015), GPR133 (Homo sapiens)-type protein (MER159334) GPR110 G-protein coupled receptor (MER159277), GPR97 protein (MER159322), KPG 006 protein (MER161773) KPG 008 protein (MER161835), KPG 009 protein (MER159335), unassigned homologue (MER166269), GPR113 protein (MER159352), brain-specific angiogenesis inhibitor 2 (MER159746), PIDD auto-processing protein unit 1 (MER020001), PIDD auto-processing protein unit 2 (MER063690), MFJC1 self-cleaving mucin (MER074260), dystroglycan (MER054741), proprotein convertase 9 (MER022416), site-1 peptidase (MEROO 1948), furin (MER000375), proprotein convertase 1 (MER000376), proprotein convertase 2 (MER000377), proprotein convertase 4 (MER028255), PACE4 proprotein convertase (MER000383), proprotein convertase 5 (MER002578), proprotein convertase 7 (MER002984), tripeptidyl-peptidase II (MER000355), subfamily S8A non-peptidase homologues (MER201339), subfamily S8A non-peptidase homologues (MER191613), subfamily S8A unassigned peptidases (MER191611), subfamily S8A unassigned peptidases (MER191612), subfamily S8A unassigned peptidases (MER191614), tripeptidyl-peptidase I (MER003575), prolyl oligopeptidase (MER000393), dipeptidyl-peptidase IV (eukaryote) (MER000401), acylaminoacyl-peptidase (MER000408), fibroblast activation protein alpha subunit (MER000399), PREPL A protein (MER004227), dipeptidyl-peptidase 8 (MER013484), dipeptidyl-peptidase 9 (MER004923), FLJ1 putative peptidase (MERO 17240), Mername-AA194 putative peptidase (MERO 17353), Memame-AA195 putative peptidase (MER017367), Mername-AA196 putative peptidase (MER017368), Mername-AA197 putative peptidase (MER017371), C14orf29 protein (MER033244), hypothetical protein (MER033245), hypothetical esterase/lipase/thioesterase (MER047309), protein bat5 (MER037840), hypothetical protein flj40219 (MER033212), hypothetical protein flj 37464 (MER033240), hypothetical protein flj33678 (MER033241), dipeptidylpeptidase homologue DPP6 (MER000403), dipeptidylpeptidase homologue DPP 10 (MER005988), protein similar to Mus musculus chromosome 20 open reading frame 135 (MER037845), kynurenine formamidase (MER046020), thyroglobulin precursor (MERO 11604), acetylcholinesterase (MER033188), cholinesterase (MER033198), carboxylesterase DI (MER033213), liver carboxylesterase (MER033220), carboxylesterase 3 (MER033224), carboxylesterase 2 (MER033226), bile salt-dependent lipase (MER033227), carboxylesterase-related protein (MER033231), neuroligin 3 (MER033232), neuroligin 4, X-linked (MER033235), neuroligin 4, Y-linked (MER033236), esterase D (MER043126), arylacetamide deacetylase (MER033237), KIAA13634ike protein (MER033242), hormone-sensitive lipase (MER033274), neuroligin 1 (MER033280), neuroligin 2 (MER033283), family S9 non peptidase homologues (MER212939), family S9 non-peptidase homologues (MER211490), subfamily S9C unassigned peptidases (MER192341), family S9 unassigned peptidases (MER209181), family S9 unassigned peptidases (MER200434), family S9 unassigned peptidases (MER209507), family S9 unassigned peptidases (MER209142), serine carboxypeptidase A (MER000430), vitellogenic carboxypeptidase-like protein (MER005492), RISC peptidase (MERO 10960), family S15 unassigned peptidases (MER199442), family S15 unassigned peptidases (MER200437), family S15 unassigned peptidases (MER212825), lysosomal Pro-Xaa carboxypeptidase (MER000446), dipeptidyl peptidase II (MER004952), thymus-specific serine peptidase (MER005538), epoxide hydrolase-like putative peptidase (MER031614), Loc328574-like protein (MER033246), abhydrolase domain-containing protein 4 (MER031616), epoxide hydrolase (MER000432), mesoderm specific transcript protein (MER 199890), mesoderm specific transcript protein (MER017123), cytosolic epoxide hydrolase (MER029997), cytosolic epoxide hydrolase (MER213866), similar to hypothetical protein FLJ22408 (MER031608), CGI-58 putative peptidase (MER030163), Williams-Beuren syndrome critical region protein 21 epoxide hydrolase (MER031610), epoxide hydrolase (MER031612), hypothetical protein flj22408 (epoxide hydrolase) (MER031617), monoglyceride lipase (MER033247), hypothetical protein (MER033249), valacyclovir hydrolase (MER033259), Ccgl-interacting factor b (MER210738).

In some embodiments, the SRS is a peptide moiety of up to 15 amino acids in length. In some embodiments, the SRS is cleaved by a protease co-localized with the target of the cell binding moiety in a tissue, and the protease cleaves the SRS in the AFFIMER® polypeptide-drug conjugate when the AFFIMER® polypeptide-drug conjugate is exposed to the protease. In some embodiments, the protease is not active or is significantly less active in tissues that do not significantly express the cell surface feature. In some embodiments, the protease is not active or is significantly less active in healthy, e.g., non-diseased tissues.
In some embodiments, the SRS is cleaved by a protease selected from the following: ADAMS or ADAMTS, e g. ADAM8, ADAM9, ADAM10, ADAM12, ADAM15, ADAM 17/T ACE, ADAMDEC1, ADAMTS 1, ADAMTS4 or ADAMTS5;
Aspartate proteases, e.g., BACE or Renin;
Aspartic cathepsins (to the extent upregulated or released by cell lysis in the extracellular space), e.g., Cathepsin D or Cathepsin E;
Caspases (to the extent upregulated or released by cell lysis in the extracellular space), e.g., Caspase 1, Caspase 2, Caspase 3, Caspase 4, Caspase 5, Caspase 6, Caspase 7, Caspase 8, Caspase 9, Caspase 10 or Caspase 14;
Cysteine cathepsins, e.g., Cathepsin B, Cathepsin C, Cathepsin K, Cathepsin L, Cathepsin S, Cathepsin V/L2, Cathepsin X/Z/P;
Cysteine proteinases, e.g., Cruzipain, Legumain or Otubain-2;
KLKs, e.g., KLK4, KLK5, KLK6, KLK7, KLK8, KLK10, KLK11, KLK13 or KLK14;
Metallo-proteinases, e.g., Meprin, Neprilysin, PSMA or BMP-1;
MMPs, e.g., MMP1, MMP2, MMP3, MMP7, MMP8, MMP9, MMP1O, MMP11, MMP12, MMP13, MMP14, MMP15, MMP16, MMP17, MMP19, MMP20, MMP23, MMP24, MMP26, MMP27;
Serine proteases, e.g., activated protein C, Cathepsin A, Cathepsin G, Chymase, coagulation factor proteases (e.g., FVIIa, FIXa, FXa, FXIa, FXIIa), Elastase, Granzyme B, Guanidinobenzoatase, HtrA1, Human Neutrophil Elastase, Lactoferrin, Marapsin, NS3/4A, PACE4, Plasmin, PSA, tPA, Thrombin, Tryptase or uPA; and/or
Type II Transmembrane Serine Proteases (TTSPs), e.g., DESC1, DPP-4, Hepsin, Matriptase-2, MT-SP1/Matriptase, DMPRSS2, DMPRSS3, DMPRSS4.

For example, suitable SRSs that can be included binder-drug conjugate, i.e., SRS is peptide moiety selected from the group consisting of: TGRGPSWV (SEQ ID NO: 1170), SARGPSRW (SEQ ID NO: 1171), TARGPSFK (SEQ ID NO: 1172), LSGRSDNH (SEQ ID NO: 1173), GGWHTGRN (SEQ ID NO: 1174), HTGRSGAL (SEQ ID NO: 1175), PLTGRSGG (SEQ ID NO: 1176), AARGPAIH (SEQ ID NO: 1177), RGPAFNPM (SEQ ID NO: 1178), SSRGPAYL (SEQ ID NO: 1179), RGPATPIM (SEQ ID NO: 1180), RGPA (SEQ ID NO: 1181), GGQPSGMWGW (SEQ ID NO: 1182), FPRPLGITGL (SEQ ID NO: 1183), VHMPLGFLGP (SEQ ID NO: 1184), SPLTGRSG (SEQ ID NO: 1185), SAGFSLPA (SEQ ID NO: 1186), LAPLGLQRR (SEQ ID NO: 1187), SGGPLGVR (SEQ ID NO: 1188), PLGL (SEQ ID NO: 1189), GPRSFGL (SEQ ID NO: 1190), and GPRSFG (SEQ ID NO: 1191).

In some embodiments, the SRS is a substrate for an MMP, such as a sequence selected from the group consisting of ISSGLLSS (SEQ ID NO: 1192), QNQALRMA (SEQ ID NO: 1193), AQNLLGMV (SEQ ID NO: 1194), STFPFGMF (SEQ ID NO: 1195), PVGYTSSL (SEQ ID NO: 1196), DWLYWPGI (SEQ ID NO: 1197), MIAPVAYR (SEQ ID NO: 1198), RPSPMWAY (SEQ ID NO: 1199), WATPRPMR (SEQ ID NO: 1200), FRLLDWQW (SEQ ID NO: 1201), LKAAPRWA (SEQ ID NO: 1202), GPSHLVLT (SEQ ID NO: 1203), LPGGLSPW (SEQ ID NO: 1204), MGLFSEAG (SEQ ID NO: 1205), SPLPLRVP (SEQ ID NO: 1206), RMHLRSLG (SEQ ID NO: 1207), LAAPLGLL (SEQ ID NO: 1208), AVGLLAPP (SEQ ID NO: 1209), LLAPSHRA (SEQ ID NO: 1210), PAGLWLDP (SEQ ID NO: 1211), and ISSGLSS (SEQ ID NO: 1212).

In some embodiments, the SRS is a substrate for an MMP, such as a sequence selected from the group consisting of ISSGLSS (SEQ ID NO: 1212), QNQALRMA (SEQ ID NO: 1193), AQNLLGMV (SEQ ID NO: 1194), STFPFGMF (SEQ ID NO: 1195), PVGYTSSL (SEQ ID NO: 1196), DWLYWPGI (SEQ ID NO: 1197), ISSGLLSS (SEQ ID NO: 1192), LKAAPRWA (SEQ ID NO: 1202), GPSHLVLT (SEQ ID NO: 1203), LPGGLSPW (SEQ ID NO: 1204), MGLFSEAG (SEQ ID NO: 1205), SPLPLRVP (SEQ ID NO: 1206), RMHLRSLG (SEQ ID NO: 1207), LAAPLGLL (SEQ ID NO: 1208), AVGLLAPP (SEQ ID NO: 1209), LLAPSHRA (SEQ ID NO: 1210), and PAGLWLDP (SEQ ID NO: 1211).

In some embodiments, the SRS is a substrate for thrombin, such as GPRSFGL (SEQ ID NO: 1190) or GPRSFG (SEQ ID NO: 1191).

### b) Spacers

In some embodiments, an AFFIMER® polypeptide-drug conjugate comprises a spacer or bond (L¹) between the half-life extension moiety and the substrate recognition sequence (SRS) cleavable by the enzyme, e.g., present in a tumor microenvironment.

The spacer may be any molecule, for example, one or more nucleotides, amino acids, chemical functional groups. In some embodiments, the spacer is a peptide linker (e.g., two or more amino acids). Spacers should not adversely affect the expression, secretion, or bioactivity of the polypeptides. In some embodiments, spacers are not antigenic and do not elicit an immune response. An immune response includes a response from the innate immune system and/or the adaptive immune system. Thus, an immune response may be a cell-mediate response and/or a humoral immune response. The immune response may be, for example, a T cell response, a B cell response, a natural killer (NK) cell response, a monocyte response, and/or a macrophage response. Other cell response are contemplated herein. In some embodiments, linkers are non-protein-coding.

In some embodiments, L¹ is a hydrocarbon (straight chain or cyclic) such as 6-maleimidocaproyl, maleimidopropanoyl and maleimidom ethyl cyclohexane-1-carboxylate, or L¹ is N-Succinimidyl 4-(2-pyridylthio)pentanoate, N-Succinimidyl 4-(N-maleimidomethyl)cyclohexane-1 carboxylate, N-Succinimidyl(4-iodo-acetyl)aminobenzoate.

In some embodiments, L¹ is a polyether such as a poly(ethylene glycol) or other hydrophilic linker. For instance, where the CBM includes a thiol (such as a cysteine residue), L¹ can be a polyethylene glycol) coupled to the thiol group through a maleimide moiety. Non-limiting examples of linkers for use in accordance with the present disclosure are described in International Publication No. WO 2019/236567, published Dec. 12, 2019, incorporated by reference herein.

### c) Self-Immolative Linkers

In some embodiments, an AFFIMER® polypeptide-drug conjugate comprises a self-immolative linker (L²) between the substrate recognition sequence (SRS) for the enzyme and the drug moiety, such as represented in the formula

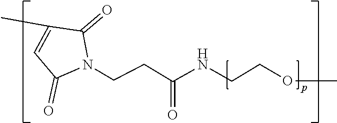

wherein, p represents an integer from 1 to 100, preferably 6 to 50, more preferably 6 to 12. In other embodiments, where the CBM includes a thiol and L¹ is a hydrocarbon moiety coupled to the thiol group through a maleimide moiety, L¹ can be represented in the formula

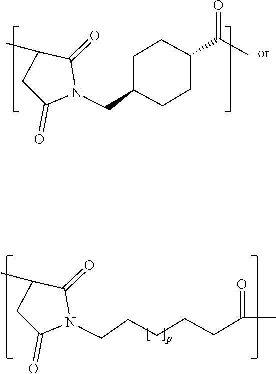

wherein, p represents an integer from 1 to 20, preferably 1 to 4. A self-immolative moiety may be defined as a bifunctional chemical group that is capable of covalently linking together two spaced chemical moieties into a normally stable molecule, releasing one of the spaced chemical moieties from the molecule by means of enzymatic cleavage; and following enzymatic cleavage, spontaneously cleaving from the remainder of the bifunctional chemical group to release the other of said spaced chemical moieties. Therefore, in some embodiments, the self-immolative moiety is covalently linked at one of its ends, directly or indirectly through a spacer unit, to the ligand by an amide bond and covalently linked at its other end to a chemical reactive site (functional group) pending from the drug moiety. The derivatization of the drug moiety with the self-immolative moiety may render the drug less pharmacologically active (e.g. less toxic) or not active at all until the drug is cleaved.

An AFFIMER® polypeptide-drug conjugate is generally stable in circulation, or at least that should be the case in the absence of an enzyme capable of cleaving the amide bond between the substrate recognition sequence (enzyme-cleavable linker) and the self-immolative moiety. Upon exposure of an AFFIMER® polypeptide-drug conjugate to a suitable enzyme, the amide bond is cleaved initiating a spontaneous self-immolative reaction resulting in the cleavage of the bond covalently linking the self-immolative moiety to the drug moiety, to thereby effect release of the free drug moiety in its underivatized or pharmacologically active form. The self-immolative moiety in conjugates either incorporate one or more heteroatoms and thereby provides improved solubility, improves the rate of cleavage and decreases propensity for aggregation of the conjugate.

In some embodiments, L² is a benzyl oxy carbonyl group. In other embodiments, the self-immolative linker L² is —NH—(CH₂)₄—C(═O)— or —NH—(CH₂)₃—C(═O)—. In yet other embodiments, the self-immolative linker L² is p-aminobenzyloxycarbonyl (PABC). In still other embodiments, the self-immolative linker L² is 2,4-bis(hydroxymeihyl) aniline.

The AFFIMER® polypeptide-drug conjugate of the present disclosure can employ a heterocyclic self-immolative moiety covalently linked to the therapeutic moiety and the cleavable substrate recognition sequence. A self-immolative moiety may be defined as a bifunctional chemical group which is capable of covalently linking together two spaced chemical moieties into a normally stable molecule, releasing one of said spaced chemical moieties from the molecule by means of enzymatic cleavage; and following said enzymatic cleavage, spontaneously cleaving from the remainder of the bifunctional chemical group to release the other of said spaced chemical moieties. In accordance with the present present disclosure, the self-immolative moiety may be covalently linked at one of its ends, directly or indirectly through a spacer unit, to the ligand by an amide bond and covalently linked at its other end to a chemical reactive site (functional group) pending from the drug. The derivatization of the therapeutic moiety with the self-immolative moiety may render the drug less pharmacologically active (e.g. less toxic) or not active at all until the drug is cleaved.

The AFFIMER® polypeptide-drug conjugate is generally stable in circulation, or at least that should be the case in the absence of an enzyme capable of cleaving the amide bond between the substrate recognition sequence and the self-immolative moiety. However, upon exposure of the AFFIMER® polypeptide-drug conjugate to a suitable enzyme, the amide bond is cleaved initiating a spontaneous self-immolative reaction resulting in the cleavage of the bond covalently linking the self-immolative moiety to the drug, to thereby effect release of the free therapeutic moiety in its underivatized or pharmacologically active form.

The self-immolative moiety in conjugates of the present disclosure, in some embodiments, either incorporate one or more heteroatoms and thereby provides improved solubility, improves the rate of cleavage and decreases propensity for aggregation of the conjugate. These improvements of the heterocyclic self-immolative linker constructs of the present disclosure over non-heterocyclic, PAB-type linkers may result in surprising and unexpected biological properties such as increased efficacy, decreased toxicity, and more desirable pharmacokinetics.

In some embodiments, L² is a benzyloxycarbonyl group.

In some embodiments, L² is

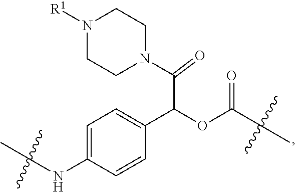

wherein R¹ is hydrogen, unsubstituted or substituted C₁₋₃ alkyl, or unsubstituted or substituted heterocyclyl. In some embodiments, R¹ is hydrogen. In some instances, R¹ is methyl.

In some embodiments, L² is selected from

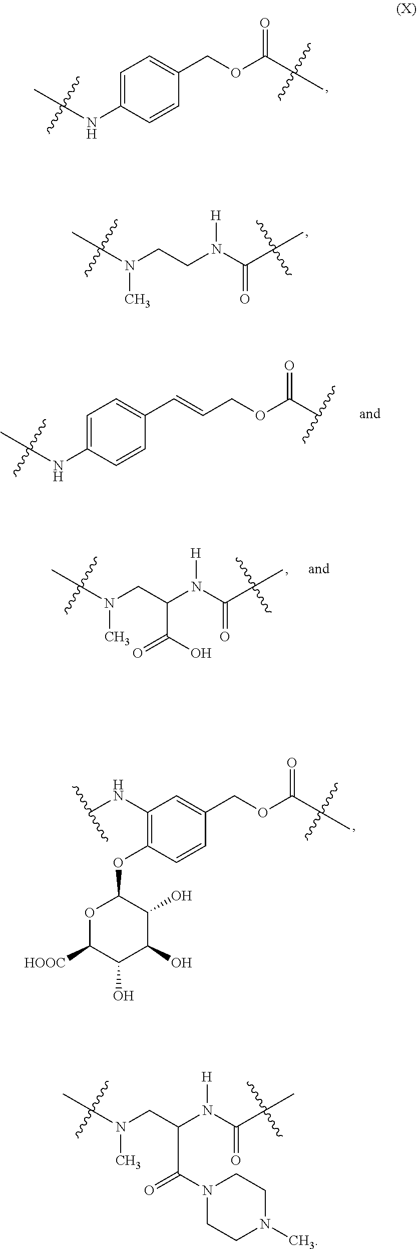

In some embodiments, the self-immolative moiety L² is selected from

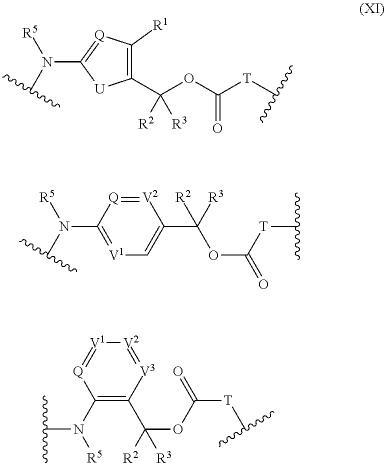

wherein
   U is O, S or NR⁶;
   Q is CR⁴ or N;
   V¹, V² and V³ are independently CR⁴ or N provided that for formula (X) and (XI) at least one of Q, V¹ and V² is N;
   T is NH, NR⁶, O or S pending from said therapeutic moiety;
   R¹, R², R³ and R⁴ are independently selected from H, F, Cl, Br, I, OH, —N(R⁵)₂, —N(R⁵)₃⁺, C₁-C₈alkylhalide, carboxylate, sulfate, sulfamate, sulfonate, —SO₂R⁵, —S(═O) R⁵, —SR⁵, —SO₂N(R⁵)₂, —C(═O)R⁵, —CO₂R⁵, —C(═O)N(R⁵)₂, —CN, —N₃, —NO₂, C₁-C₈alkoxy, C₁-C₈ halosubstituted alkyl, polyethyleneoxy, phosphonate, phosphate, C₁-C₈alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; or when taken together, R² and R³ form a carbonyl (═O), or spiro carbocyclic ring of 3 to 7 carbon atoms; and R⁵ and R⁶ are independently selected from H, C₁-C₈ alkyl, C₁-C₈substituted alkyl, C₂-C₈ alkenyl, C₂-C₈ substituted alkenyl, C₂-C₈alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; where C₁-C₈ substituted alkyl, C₂-C₈ substituted alkenyl, C₂-C₈substituted alkynyl, C₆-C₂₀ substituted aryl, and C₂-C₂₀ substituted heterocycle are independently substituted with one or more substituents selected from F, Cl, Br, I, OH, —N(R⁵)₂, —N(R⁵)₃⁺, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O) R⁵, —SR⁵, —SO₂N(R⁵)₂, —C(═O) R⁵, —CO₂R⁵, —C(═O)N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂₀ heterocycle, polyethyleneoxy, phosphonate, and phosphate.

It will be understood that when T is NH, it is derived from a primary amine (—NH2) pending from the therapeutic moiety (prior to coupling to the self-immolative moiety) and when Tis N, it is derived from a secondary amine (—NH—) from the therapeutic moiety (prior to coupling to the self-immolative moiety). Similarly, when T is O or S, it is derived from a hydroxyl (—OH) or sulfhydryl (—SH) group respectively pending from the therapeutic moiety prior to coupling to the self-immolative moiety.

In some embodiments, the self-immolative linker L² is —NH—(CH₂)₄—C(═O)— or —NH—(CH₂)₃—C(═O)—.

In some embodiments, the self-immolative linker L² is p-aminobenzyloxycarbonyl (PABC).

In some embodiments, the self-immolative linker L² is 2,4-bis(hydroxymethyl) aniline.

Other examples of self-immolative linkers that are readily adapted for use in AFFIMER® polypeptide-drug conjugates described herein are taught in, for example, U.S. Pat. No. 7,754,681; WO 2012/074693A1; U.S. Pat. No. 9,089,614; EP 1,732,607; WO 2015/038426A1 (all of which are incorporated by reference); Walther et al. “Prodrugs in medicinal chemistry and enzyme prodrug therapies” Adv Drug Deliv Rev. 2017 Sep. 1; 118:65-77; and Tranoy-Opalinski et al. “Design of self-immolative linkers for tumor-activated prodrug therapy”, Anticancer Agents Med Chem. 2008 August; 8 (6): 618-37; the teachings of each of which are incorporated by reference herein.

Yet other non-limiting examples of self-immolative linkers for use in accordance with the present disclosure are described in International Publication No. WO 2019/236567, published Dec. 12, 2019, incorporated by reference herein.

### IV. Encoded AFFIMER® Construct for In Vivo Delivery

An alternative approach to the delivery of therapeutic AFFIMER® agents, such as a PD-L1 AFFIMER® agent, would be to leave the production of the therapeutic polypeptide to the body itself. A multitude of clinical studies have illustrated the utility of in vivo gene transfer into cells using a variety of different delivery systems. In vivo gene transfer seeks to administer to patients the encoded AFFIMER® construct, rather than the AFFIMER® agent. This allows the patient's body to produce the therapeutic AFFIMER® agent of interest for a prolonged period of time, and secrete it either systemically or locally, depending on the production site. Gene-based encoded AFFIMER® construct can present a labor- and cost-effective alternative to the conventional production, purification and administration of the polypeptide version of the AFFIMER® agent. A number of antibody expression platforms have been pursued in vivo to which delivery of encoded AFFIMER® construct can be adapted: these include viral vectors, naked DNA and RNA. encoded AFFIMER® construct gene transfer can not only enable cost-savings by reducing the cost of goods and of production but may also be able to reduce the frequency of drug administration. Overall, a prolonged in vivo production of the therapeutic AFFIMER® agent by expression of the encoded AFFIMER® construct can contribute to (i) a broader therapeutic or prophylactic application of AFFIMER® agents in price-sensitive conditions, (ii) an improved accessibility to therapy in both developed and developing countries, and (iii) more effective and affordable treatment modalities. In addition to in vivo gene transfer, cells can be harvested from the host (or a donor), engineered with encoded AFFIMER® construct sequences to produce AFFIMER® agents and re-administered to patients.

Intramuscular antibody gene administration has been most widely evaluated (reviewed in Deal et al. (2015) “Engineering humoral immunity as prophylaxis or therapy” Curr Opin Immunol. 35:113-22), and also carries the highest clinical translatability and application when applied to encoded AFFIMER® construct. Indeed, the inherent anatomical, cellular and physiological properties of skeletal muscle make it a stable environment for long-term encoded AFFIMER® construct expression and systemic circulation. Skeletal muscle is easily accessible, allowing multiple or repeated administrations. The abundant blood vascular supply provides an efficient transport system for secreted therapeutic AFFIMER® agents into the circulation. The syncytial nature of muscle fibers allows dispersal of nucleotides from a limited site of penetration to a large number of neighboring nuclei within the fiber. Skeletal muscle fibers are also terminally differentiated cells, and nuclei within the fibers are post-mitotic. Consequently, integration in the host genome is not a prerequisite to attain prolonged monoclonal antibody (mAb) expression. The liver is another site often used for pre-clinical antibody gene transfer and is typically transfected via intravenous (i.v.) injection and can also be a site of gene transfer for encoded AFFIMER® construct either for local delivery of AFFIMER® agents (such as in the treatment of liver cancer and/or metaplasias) or for the generation of AFFIMER® agents that are secreted into the vascular for systemic circulation. This organ has various physiological functions, including the synthesis of plasma proteins. This organ can be particularly well suited for in vivo encoded AFFIMER® construct expression.

The tumor presents another site for encoded AFFIMER® construct transfer, targeted either via i.v. or direct injection/electroporation. Indeed, intratumoral encoded AFFIMER® construct expression can allow for a local production of the therapeutic AFFIMER® agents, waiving the need for high systemic AFFIMER® agent levels that might otherwise be required to penetrate and impact solid tumors. A similar rationale applies for the brain, which is frequently targeted in the context of antibody gene transfer to avoid the difficulties with blood-brain barrier trafficking and would likewise be a target for delivery of encoded AFFIMER® construct. See, for example, Beckman et al. (2015) “Antibody constructs in cancer therapy: protein engineering strategies to improve exposure in solid tumors” Cancer 109 (2): 170-9; Dronca et al. (2015) “Immunomodulatory antibody therapy of cancer: the closer, the better” Clin Cancer Res. 21 (5): 944 6; and Neves et al. (2016) “Antibody approaches to treat brain diseases” Trends Biotechnol. 34 (1): 36-48.

The success of gene therapy has largely been driven by improvements in nonviral and viral gene transfer vectors. An array of physical and chemical nonviral methods have been used to transfer DNA and mRNA to mammalian cells and a substantial number of these have been developed as clinical stage technologies for gene therapy, both ex vivo and in vivo, and are readily adapted for delivery of the encoded AFFIMER® construct of the present disclosure. To illustrate, cationic liposome technology can be employed, which is based on the ability of amphipathic lipids, possessing a positively charged head group and a hydrophobic lipid tail, to bind to negatively charged DNA or RNA and form particles that generally enter cells by endocytosis. Some cationic liposomes also contain a neutral co-lipid, thought to enhance liposome uptake by mammalian cells. See, for example, Felgner et al. (1987) Lipofection: a highly efficient, lipid-mediated DNA-transfection procedure. MNAS 84:7413-7417; San et al. (1983) “Safety and short-term toxicity of a novel cationic lipid formulation for human gene therapy” Hum. Gene Ther. 4:781-788; Xu et al. (1996) “Mechanism of DNA release from cationic liposome/DNA complexes used in cell transfection” Biochemistry 35:5616-5623; and Legendre et al. (1992) “Delivery of plasmid DNA into mammalian cell lines using pH-sensitive liposomes: comparison with cationic liposomes” Pharm. Res. 9, 1235-1242.

Similarly, other polycations, such as poly-l-lysine and polyethylene-imine, can be used to deliver encoded AFFIMER® construct. These polycations complex with nucleic acids via charge interaction and aid in the condensation of DNA or RNA into nanoparticles, which are then substrates for endosome-mediated uptake. Several of these cationic nucleic acid complex technologies have been developed as potential clinical products, including complexes with plasmid DNA, oligodeoxynucleotides, and various forms of synthetic RNA. Modified (and unmodified or “naked”) DNA and RNA have also been shown to mediate successful gene transfer in a number of circumstances and can also be used as systems for delivery of encoded AFFIMER® construct. These include the use of plasmid DNA by direct intramuscular injection, the use of intratumoral injection of plasmid DNA. See, for example, Rodrigo et al. (2012) “De novo automated design of small RNA circuits for engineering synthetic riboregulation in living cells” PNAS 109:15271-15276; Oishi et al. (2005) “Smart polyion complex micelles for targeted intracellular delivery of PEGylated antisense oligonucleotides containing acid-labile linkages” Chembiochem. 6:718-725; Bhatt et al. (2015) “Microbeads mediated oral plasmid DNA delivery using polymethacrylate vectors: an effectual groundwork for colorectal cancer” Drug Deliv. 22:849-861; Ulmer et al. (1994) Protective immunity by intramuscular injection of low doses of influenza virus DNA vaccines” Vaccine 12:1541-1544; and Heinzerling et al. (2005) “Intratumoral injection of DNA encoding human interleukin 12 into patients with metastatic melanoma: clinical efficacy” Hum. Gene Ther. 16:35-48.

Viral vectors are currently used as a delivery vehicle in the vast majority of pre-clinical and clinical gene therapy trials and in the first to be approved directed gene therapy. See Gene Therapy Clinical Trials Worldwide 2017 (abedia.com/wiley/). The main driver thereto is their exceptional gene delivery efficiency, which reflects a natural evolutionary development; viral vector systems are attractive for gene delivery, because viruses have evolved the ability to cross through cellular membranes by infection, thereby delivering nucleic acids such as encoded AFFIMER® construct to target cells. Pioneered by adenoviral systems, the field of viral vector-mediated antibody gene transfer made significant strides in the past decades. The myriad of successfully evaluated administration routes, pre-clinical models and disease indications puts the capabilities of antibody gene transfer at full display through which the skilled artisan would readily be able to identify and adapt antibody gene transfer systems and techniques for in vivo delivery of encoded AFFIMER® polypeptides. Muscle has emerged as the administration site of choice for prolonged mAb expression and would similarly be a suitable target tissue for prolonged AFFIMER® agent expression. In the context of vectored intratumoral encoded AFFIMER® construct gene transfer, oncolytic viruses have a distinct advantage, as they can specifically target tumor cells, boost AFFIMER® agent expression, and amplify therapeutic responses-such as to a PD-L1 AFFIMER® agent.

In vivo gene transfer of encoded AFFIMER® construct can also be accomplished by use of nonviral vectors, such as expression plasmids. Nonviral vectors are easily produced and do not seem to induce specific immune responses. Muscle tissue is most often used as target tissue for transfection because muscle tissue is well vascularized and easily accessible, and myocytes are long-lived cells. Intramuscular injection of naked plasmid DNA results in transfection of a certain percentage of myocytes. Using this approach, plasmid DNA encoding cytokines and cytokine/IgG1 chimeric proteins has been introduced in vivo and has positively influenced (autoimmune) disease outcome.

In some instances, in order to increase transfection efficiency via so-called intravascular delivery in which increased gene delivery and expression levels are achieved by inducing a short-lived transient high pressure in the veins. Special blood-pressure cuffs that may facilitate localized uptake by temporarily increasing vascular pressure and can be adapted for use in human patients for this type of gene delivery. See, for example, Zhang et al. (2001) “Efficient expression of naked DNA delivered intraarterially to limb muscles of nonhuman primates” Hum. Gene Ther., 12:427-438

Increased efficiency can also be gained through other techniques, such as in which delivery of the nucleic acid is improved by use of chemical carriers-cationic polymers or lipids—or via a physical approach—gene gun delivery or electroporation. See Tranchant et al. (2004) “Physicochemical optimization of plasmid delivery by cationic lipids” J. Gene Med., 6 (Suppl. 1): S24-S35; and Niidome et al. (2002) “Gene therapy progress and prospects: nonviral vectors” Gene Ther., 9:1647-1652. Electroporation is especially regarded as an interesting technique for nonviral gene delivery. Somiari, et al. (2000) “Theory and in vivo application of electroporative gene delivery” Mol. Ther. 2:178-187; and Jaroszeski et al. (1999) “In vivo gene delivery by electroporation” Adv. Drug Delivery Rev., 35:131-137. With electroporation, pulsed electrical currents are applied to a local tissue area to enhance cell permeability, resulting in gene transfer across the membrane. Research has shown that in vivo gene delivery can be at least 10-100 times more efficient with electroporation than without. See, for example, Aihara et al. (1998) “Gene transfer into muscle by electroporation in vivo” Nat. Biotechnol. 16:867-870; Mir, et al. (1999) “High-efficiency gene transfer into skeletal muscle mediated by electric pulses” PNAS 96:4262-4267; Rizzuto, et al. (1999) “Efficient and regulated erythropoietin production by naked DNA injection and muscle electroporation” PNAS 96:6417-6422; and Mathiesen (1999) “Electropermeabilization of skeletal muscle enhances gene transfer in vivo” Gene Ther., 6:508-514.

Encoded PD-L1 AFFIMER® polypeptides can be delivered by a wide range of gene delivery system commonly used for gene therapy including viral, non-viral, or physical. See, for example, Rosenberg et al., Science, 242:1575-1578, 1988, and Wolff et al., Proc. Natl. Acad. Sci. USA 86:9011-9014 (1989). Discussion of methods and compositions for use in gene therapy include Eck et al., in Goodman & Gilman's The Pharmacological Basis of Therapeutics, Ninth Edition, Hardman et al., eds., McGraw-Hill, New York, (1996), Chapter 5, pp. 77-101; Wilson, Clin. Exp. Immunol. 107 (Suppl. 1): 31-32, 1997; Wivel et al., Hematology/Oncology Clinics of North America, Gene Therapy, S. L. Eck, ed., 12 (3): 483-501, 1998; Romano et al., Stem Cells, 18:19-39, 2000, and the references cited therein. U.S. Pat. No. 6,080,728 also provides a discussion of a wide variety of gene delivery methods and compositions. The routes of delivery include, for example, systemic administration and administration in situ.

An effective encoded AFFIMER® construct gene transfer approach must be directed to the specific tissues/cells where it is needed, and the resulting transgene expression should be at a level that is appropriate to the specific application. Promoters are a major cis-acting element within the vector genome design that can dictate the overall strength of expression as well as cell-specificity.

TABLE 8
Exemplary Ubiquitous and Cell-specific Promoters.

| | | Relative | Size | |
|---|---|---|---|---|
| Promoter | Specificity | Strength | (bps) | Reference(s) |
| CMV | Ubiquitous | +++ | 750-800 | Xu et al. Gene Ther. 2001 8: 1323-1332; |
| | | | | Gray et al., Hum Gene Ther. 2011 |
| | | | | 22: 1143-1153 |
| CBA (including | Ubiquitous | +++ | 248-1,600 | Klein et al. Exp Neurol. 2002 176(1): |
| derivatives: | | | | 66-74; Ohlfest et al. Blood. 2005 105: |
| CAG, CBh, etc.) | | | | 2691-2698; and Gray et al. Hum Gene |
| | | | | Ther. 2011 22: 1143-1153. |
| EF-1α | Ubiquitous | ++ | 2,500 | Gill et al. Gene Ther. 2001 8(20): |
| | | | | 1539-1546; Xu et al. Gene Ther. 2001 |
| | | | | 8: 1323-1332; and Gilham et al. J Gene |
| | | | | Med. 2010 12(2): 129-136. |
| PGK | Ubiquitous | ++ | 426 | Gilham et al. J Gene Med. 2010 |
| | | | | 12(2): 129-136. |
| UBC | Ubiquitous | + | 403 | Gill et al. Gene Ther. 2001 8(20): |
| | | | | 1539-1546; Qin et al. PLoS One. 2010 |
| | | | | 5(5): e10611. |
| GUSB (hGBp) | Ubiquitous | + | 378 | Husain et al. Gene Ther. 2009 |
| | | | | 16: 927-932. |
| UCOE | Ubiquitous | ++ | 600-2,500 | Antoniou et al. Hum Gene Ther. 2013 |
| (Promoter of | | | | 24(4): 363-374. |
| HNRPA2B1- | | | | |
| CBX3) | | | | |
| hAAT | Liver | ++ | 347-1,500 | Van Linthout et al. Hum Gene Ther. |
| | | | | 2002 13(7): 829-840; Cunningham et |
| | | | | al. Mol Ther. 2008 16(6): 1081-1088 |
| TBG | Liver | ++ | 400 | Yan et al. Gene. 2012 506(2): 289-294. |
| Desmin | Skeletal muscle | +++ | 1,700 | Talbot et al. Mol Ther. 2010 18: 601-608. |
| MCK | Skeletal muscle | ++ | 595-1,089 | Talbot et al. Mol Ther. 2010 18: 601-608; |
| | | | | Wang et al. Gene Ther. 2008 15: |
| | | | | 1489-1499; Katwal et al. Gene Ther. |
| | | | | 2013 20(9): 930-938. |
| C5-12 | Skeletal, cardiac, | ++ | 312 | Wang et al. Gene Ther. 2008 |
| | and diaphragm | | | 15: 1489-1499 |
| NSE | Neuron | +++ | 300-2,200 | Xu et al. Gene Ther. 2001 8: 1323-1332 |
| Synapsin | Neuron | + | 470 | Kügler et al. Virology. 2003 311: 89-95; |
| | | | | Hioki et al. Gene Ther. 2007 14: |
| | | | | 872-882; Kuroda et al. J Gene Med. |
| | | | | 2008 10: 1163-1175. |
| PDGF | Neuron | +++ | 1,400 | Patterna et al. Gene Ther. 2000 7(15): |
| | | | | 1304-1311; Hioki et al. Gene Ther. |
| | | | | 2007 14: 872-882 |
| MecP2 | Neuron | + | 229 | Rastegar et al. LoS One. 2009 4: e6810; |
| | | | | Gray et al., Hum Gene Ther. 2011 22: |
| | | | | 1143-1153 |
| CaMKII | Neuron | ++ | 364-2,300 | Hioki et al. Gene Ther. 2007 14: |
| | | | | 872-882; Kuroda et al. J Gene Med. |
| | | | | 2008 10: 1163-1175 |
| mGluR2 | Neuron | + | 1,400 | Brené et al. Eur J Neurosci. 2000 12: |
| | | | | 1525-1533; Kuroda et al. J Gene Med. |
| | | | | 2008 10: 1163-1175 |
| NFL | Neuron | + | 650 | Xu et al. Gene Ther. 2001 8: 1323-1332 |
| NFH | Neuron | + | 920 | Xu et al. Gene Ther. 2001 8: 1323-1332 |
| nβ2 | Neuron | + | 650 | Xu et al. Gene Ther. 2001 8: 1323-1332 |
| PPE | Neuron | + | 2700 | Xu et al. Gene Ther. 2001 8: 1323-1332 |
| Enk | Neuron | + | 412 | Xu et al. Gene Ther. 2001 8: 1323-1332 |
| EAAT2 | Neuron and | ++ | 966 | Su et al. Proc Natl Acad Sci U S A. |
| | astrocyte | | | 2003 100: 1955-1960; Kuroda et al. J |
| | | | | Gene Med. 2008 10: 1163-1175 |
| GFAP | Astrocyte | ++ | 681-2,200 | Brenner et al. J Neurosci. 1994 14: |
| | | | | 1030-1037; Xu et al. Gene Ther. 2001 |
| | | | | 8: 1323-1332; Lee et al. Glia. 2008 56: |
| | | | | 481-493; Dirren et al. Hum Gene Ther. |
| | | | | 2014 25: 109-120 |
| MBP | Oligodendrocytes | ++ | 1,900 | Chen et al. Gene Ther. 1998 5(1): 50-58 |

In some cases, ubiquitous expression of the encoded AFFIMER® construct in all cell types is desired. Constitutive promoters such as the human elongation factor 1α-subunit (EF1α), immediate-early cytomegalovirus (CMV), chicken β-actin (CBA) and its derivative CAG, the β glucuronidase (GUSB), or ubiquitin C (UBC) can be used to promote expression of the encoded AFFIMER® construct in most tissues. Generally, CBA and CAG promote the larger expression among the constitutive promoters; however, their size of ˜1.7 kbs in comparison to CMV (˜0.8 kbs) or EF1α (˜1.2 kbs) may limit use in vectors with packaging constraints such as AAV, particularly where AFFIMER® agent produced by expression of the encoded AFFIMER® construct is large. The GUSB or UBC promoters can provide ubiquitous gene expression with a smaller size of 378 bps and 403 bps, respectively, but they are considerably weaker than the CMV or CBA promoter. Thus, modifications to constitutive promoters in order to reduce the size without affecting its expression have been pursued and examples such as the CBh (˜800 bps) and the miniCBA (˜800 bps) can promote expression comparable and even higher in selected tissues (Gray et al., Hum Gene Ther. 2011 22:1143-1153).

When expression of the encoded AFFIMER® construct should be restricted to certain cell types within an organ, promoters can be used to mediate this specificity. For example, within the nervous system promoters have been used to restrict expression to neurons, astrocytes, or oligodendrocytes. In neurons, the neuron-specific enolase (NSE) promoter drives stronger expression than ubiquitous promoters. Additionally, the platelet-derived growth factor B-chain (PDGF-β), the synapsin (Syn), and the methyl-CpG binding protein 2 (MeCP2) promoters can drive neuron-specific expression at lower levels than NSE. In astrocytes, the 680 bps-long shortened version [gfaABC (1) D] of the glial fibrillary acidic protein (GFAP, 2.2 kbs) promoter can confer higher levels of expression with the same astrocyte-specificity as the GFAP promoter. Targeting oligodendrocytes can also be accomplished by the selection of the myelin basic protein (MBP) promoter, whose expression is restricted to this glial cell; however, its size of 1.9 kbs and low expression levels limit its use.

In the case of expressing the encoded AFFIMER® construct in skeletal muscle cells, exemplary promoters based on muscle creatine kinase (MCK) and desmin (1.7 kbs) have showed a high rate of specificity (with minimal expression in the liver if desired). The promoter of the α-myosin heavy chain (α-MHC; 1.2 kbs) has shown significant cardiac specificity in comparison with other muscle promoters (Lee et al., 2011 J Cardiol. 57 (1): 115-22). In hematopoietic stem cells the synthetic MND promoter (Li et al., 2010 J Neurosci Methods. 189 (1): 56-64) and the promoter contained in the 2AUCOE (ubiquitous chromatin opening element) have shown to drive a higher transgene expression in all cell lineages when compared to the EF1α and CMV promoters, respectively (Zhang et al., 2007 Blood. 110 (5): 1448-57; Koldej 2013 Hum Gene Ther Clin Dev. 24 (2): 77-85; Dighe et al., 2014 PLOS One. 9 (8): e104805). Conversely, using promoters to restrict expression to only liver hepatocytes after vector-mediated gene transfer has been shown to reduce transgene-specific immune responses in systems where that is a risk, and to even induce immune tolerance to the expressed protein (Zhang et al., 2012 Hum Gene Ther. 23 (5): 460-72), which for certain AFFIMER® agents may be beneficial. The α1-antitrypsin (hAAT; 347 bps) and the thyroxine binding globulin (TBG; ˜ 400 bps) promoters drive gene expression restricted to the liver with minimal invasion to other tissues (Yan et al., 2012 Gene. 506 (2): 289-94; Cunningham et al., 2008 Mol Ther. 16 (6): 1081-8).

In some embodiments, a mechanism to control the duration and amount of in vivo encoded AFFIMER® construct expression will typically be desired. There are a variety of inducible promoters which can be adapted for use with viral vectored- and plasmid DNA-based encoded AFFIMER® construct gene transfer. See Fang et al. (2007) “An antibody delivery system for regulated expression of therapeutic levels of monoclonal antibodies in vivo” Mol Ther. 5 (6): 1153-9; and Perez et al. (2004) “Regulatable systemic production of monoclonal antibodies by in vivo muscle electroporation” Genet Vaccines Ther. 2 (1): 2. An exemplary a regulatable mechanism currently under clinical evaluation is an ecdysone-based gene switch activated by a small molecule ligand. Cai et al. (2016) “Plasma pharmacokinetics of veledimex, a small-molecule activator ligand for a proprietary gene therapy promoter system, in healthy subjects” Clin Pharmacol Drug Dev. 2016.

In some embodiments of an encoded AFFIMER® construct, viral post-transcriptional regulatory elements (PREs) may be used; these cis-acting elements are required for nuclear export of intronless viral RNA (Huang and Yen, 1994 J Virol. 68 (5): 3193-9; and 1995 Mol Cell Biol. 15 (7): 3864-9). Examples include HPRE (Hepatitis B Virus PRE, 533 bps) and WPRE (Woodchuck Hepatitis Virus PRE, 600 bps), which can increase the level of transgene expression by almost 10-fold in certain instances (Donello et al., 1998 J Virol. 72 (6): 5085-92). To further illustrate, using lentiviral and AAV vectors, WPRE was found to increase CMV promoter driven transgene expression, as well as increase PPE, PDGF and NSE promoter-driven transgene expression. Another effect of the WPRE can be to protect encoded AFFIMER® transgenes from silencing (Paterna et al., 2000 Gene Ther. 7 (15): 1304-11; Xia et al., 2007 Stem Cells Dev. 2007 February; 16 (1): 167-76).

The polyadenylation of a transcribed encoded AFFIMER® construct transcript can also be important for nuclear export, translation, and mRNA stability. Therefore, in some embodiments, the encoded AFFIMER® construct will include a polyadenylation signal sequence. A variety of studies are available that have determined the effects of different poly A signals on gene expression and mRNA stability. Exemplary polyadenylation signal sequences include SV40 late or bovine growth hormone polyA (bGHpA) signal sequences, as well as minimal synthetic polyA (SPA) signal (Levitt et al., 1989 Genes Dev. 3 (7): 1019-25; Yew et al., 1997 Hum Gene Ther. 1997 8 (5): 575-84). The efficiency of polyadenylation is increased by the SV40 late polyA signal upstream enhancer (USE) placed upstream of other polyA signals (Schek et al., 1992 Mol Cell Biol. 12 (12): 5386-93). In some embodiments, merely to illustrate, the encoded AFFIMER® construct will include an SV40 late+2×USE polyA signal.

TABLE 9
Exemplary Polyadenylation Signals

| PolyA Signal | Relative | Size | | |
|---|---|---|---|---|
| and USE | Strength | (bps) | Source | Reference(s) |
| hGH | + | 624 | Human growth | Ostedgaard et al. Proc Natl Acad Sci |
| | | | hormone | U S A. 2005 102(8): 2952-2957 |
| SV40 late | +++ | 135 | Simian virus 40 | Choi et al. Mol Brain. 2014 7: 17 |
| SPA (synthetic | + | 49 | Rabbit β-globin | Levitt et al. Genes Dev. 3(7): 1019-1025; |
| polyA) | | | | Yew et al. Hum Gene Ther. 1997 8(5): |
| | | | | 575-584; Ostedgaard et al. Proc Natl |
| | | | | Acad Sci U S A. 2005 102(8): 2952-2957; |
| | | | | Choi et al. Mol Brain. 2014 7: 17 |
| bGH | ++ | 250 | Bovine growth | Yew et al. Hum Gene Ther. 1997 8(5): |
| | | | hormone | 575-584; Xu et al. Gene Ther. 2001 8: |
| | | | | 1323-1332; Wu et al. Mol Ther. 2008 |
| | | | | 16(2): 280-289; Gray et al., Hum Gene |
| | | | | Ther. 2011 22: 1143-1153; Choi et al. |
| | | | | Mol Brain. 2014 7: 17 |
| SV40 late | ++ | 100 | Simian virus 40 | Schambach et al. Mol Ther. 2007 |
| 2xUSE | | | | 15(6): 1167-1173; Choi et al. Mol |
| | | | | Brain. 2014 7: 17 |
| HIV-1 USE | + | 35 | Human | Schambach et al. Mol Ther. 2007 |
| | | | immunodeficiency | 15(6): 1167-1173 |
| | | | virus 1 | |
| GHV USE | + | 39 | Ground squirrel | Schambach et al. Mol Ther. 2007 |
| | | | hepatitis virus | 15(6): 1167-1173 |
| Adenovirus | + | 21 | Adenovirus | Schambach et al. Mol Ther. 2007 |
| (L3) USE | | | | 15(6): 1167-1173 |
| hTHGB | + | 21 | Human | Schambach et al. Mol Ther. 2007 |
| USE | | | prothrombin | 15(6): 1167-1173 |
| hC2 USE | + | 53 | Human C2 | Schambach et al. Mol Ther. 2007 |
| | | | complement gene | 15(6): 1167-1173 |

In some embodiments, it may be desirable for the encoded AFFIMER® construct to include at least one regulatory enhancers, e.g., in addition to any promoter sequences. The CMV enhancer is upstream of the CMV promoter at −598 to −68 (Boshart et al., 1985 Cell. 41 (2): 521-30) (˜600 bps) and contains transcription binding sites. In some embodiments, a CMV enhancer can be included in the construct to increase tissue-specific promoter-driven transgene expression, such as using the ANF (atrial natriuretic factor) promoter, the CC10 (club cell 10) promoter, SP-C (surfactant protein C) promoter, or the PDGF-β (platelet-derived growth factor-β) promoter (merely as examples). Altogether, the CMV enhancer increases transgene expression under different cell-specific promoters and different cell types making it a broadly applicable tool to increase transgene expression levels. In muscle, for example, in AAV expression systems transgene expression using the CMV enhancer with a muscle-specific promoter can increase expression levels of the protein encoded by the transgene, so would be particularly useful in the current disclosure for expressing AFFIMER® agents from encoded AFFIMER® constructs introduced into muscle cells of a patient.

The encoded AFFIMER® agents may also include at least one intronic sequence. The presence of an intron or intervening sequence in mRNA was first described, in vitro, to be important for mRNA processing and increased transgene expression (Huang and Gorman, 1990 Mol Cell Biol. 10 (4): 1805-10; Niwa et al., 1990 Genes Dev. 4 (9): 1552-9). The intron(s) can be placed within the coding sequence for the AFFIMER® agent and/or can be placed between the promoter and transgene. A variety of introns (Table 10) placed between the promoter and transgene were compared, in mice using AAV2, for liver transgene expression (Wu et al., 2008). The MVM (minute virus of mice) intron increased transgene expression more than any other intron tested and more than 80-fold over no intron (Wu et al., 2008). However, in cultured neurons using AAV expression cassettes, transgene expression was less under a CaMPKII promoter with a chimeric intron (human β-globin donor and immunoglobulin heavy chain acceptor) between the transgene and polyA signal compared to a WPRE (Choi et al., 2014). Together, an intron can be a valuable element to include in an expression cassette to increase transgene expression.

TABLE 10
Exemplary Introns

| | Relative | Size | | |
|---|---|---|---|---|
| Itron | Strength | (bps) | Source | Reference(s) |
| MVM | +++ | 67-97 | Minute virus | Wu et al. Mol Ther. 2008 16(2): 280-289 |
| | | | of mice | |
| F.IX truncated | + | 300 | Human | Wu et al. Mol Ther. 2008 16(2): 280-289; |
| intron 1 | | | factor IX | Kurachi et al. J Biol Chem. 1995 |
| | | | | 270(10): 5276-5281 |
| β-globin SD/ | + | 250 | Human, | Wu et al. Mol Ther. 2008 16(2): 280-289; |
| immunoglobin heavy | | | pZac2.1 | Choi et al. Mol Brain. 2014; 7: 17 |
| chain SA | | | | |
| Adenovirus SD^{#}/ | ++ | 500 | pAdβ | Wong et al. Chromosoma. 1985 92(2): |
| immunoglobulin SA* | | | | 124-135; Yew et al. Hum Gene Ther. |
| | | | | 1997 8(5): 575-584 |
| SV40 late SD^{#}/SA* | + | 180 | pCMVβ | Yew et al. Hum Gene Ther. 1997 |
| (19S/16S) | | | | 8(5): 575-584 |
| Hybrid adenovirus | +++ | 230 | Adenovirus | Choi et al. Mol Brain. 2014; 7: 17; |
| SD^{#}/IgG SA* | | | | Huang et al. Mol Cell Biol. 1990 |
| | | | | 10(4): 1805-1810 |

In the case of episomal vectors, the encoded AFFIMER® constructs may also include at least one origin of replication, minichromosome maintenance elements (MME) and/or nuclear localization elements. Episomal vectors of the disclosure comprise a portion of a virus genomic DNA that encodes an origin of replication (ori), which is required for such vectors to be self-replicating and, thus, to persist in a host cell over several generations. In addition, an episomal vector of the disclosure also may contain at least one gene encoding at least one viral protein required for replication, e.g., replicator protein(s). Optionally, the replicator protein(s) which help initiate replication may be expressed in trans on another DNA molecule, such as on another vector or on the host genomic DNA, in the host cell containing a self-replicating episomal expression vector of this disclosure. Preferred self-replicating episomal LCR-containing expression vectors of the disclosure do not contain viral sequences that are not required for long-term stable maintenance in a eukaryotic host cell such as regions of a viral genome DNA encoding core or capsid proteins that would produce infectious viral particles or viral oncogenic sequences which may be present in the full-length viral genomic DNA molecule. The term “stable maintenance” herein, refers to the ability of a self-replicating episomal expression vector of this disclosure to persist or be maintained in non-dividing cells or in progeny cells of dividing cells in the absence of continuous selection without a significant loss (e.g., >50%) in copy number of the vector for two, three, four, or five or more generations. In some embodiments, the vectors will be maintained over 10-15 or more cell generations. In contrast, “transient” or “short-term” persistence of a plasmid in a host cell refers to the inability of a vector to replicate and segregate in a host cell in a stable manner; that is, the vector will be lost after one or two generations or will undergo a loss of >51% of its copy number between successive generations.

Several representative self-replicating, LCR-containing, episomal vectors useful in the context of the present disclosure are described further below. The self-replicating function may alternatively be provided by at least one mammalian sequence such as described by Wohlgeuth et al., 1996, Gene Therapy 3:503; Vos et al., 1995, Jour. Cell. Biol., Supp. 21A, 433; and Sun et al., 1994, Nature Genetics 8:33, optionally in combination with at least one sequence that may be required for nuclear retention. The advantage of using mammalian, especially human sequences for providing the self-replicating function is that no extraneous activation factors are required which could have toxic or oncogenic properties. It will be understood by one of skill in the art that the disclosure is not limited to any one origin of replication or any one episomal vector but encompasses the combination of the tissue-restricted control of an LCR in an episomal vector. See also WO1998007876 “Self-replicating episomal expression vectors conferring tissue-specific gene expression” and U.S. Pat. No. 7,790,446 “Vectors, cell lines and their use in obtaining extended episomal maintenance replication of hybrid plasmids and expression of gene products”

Epstein-Barr Virus-Based Self-Replicating Episomal Expression Vectors. The latent origin oriP from Epstein-Barr Virus (EBV) is described in Yates et. al., Proc. Natl. Acad. Sci. USA 81:3806-3810 (1984); Yates et al., Nature 313:812-815 (1985); Krysan et al., Mol. Cell. Biol. 9:1026-1033 (1989); James et al. Gene 86:233-239 (1990), Peterson and Legerski, Gene 107:279-284 (1991); and Pan et al., Som. Cell Molec. Genet. 18:163-177 (1992)). An EBV-based episomal vector useful according to the disclosure can contain the oriP region of EBV which is carried on a 2.61 kb fragment of EBV and the EBNA-1 gene which is carried on a 2.18 kb fragment of EBV. The EBNA-1 protein, which is the only viral gene product required to support in trans episomal replication of vectors containing oriP, may be provided on the same episomal expression vector containing oriP. It is also understood, that as with any protein such as EBNA-1 known to be required to support replication of viral plasmid in trans, the gene also may be expressed on another DNA molecule, such as a different DNA vector.

Papilloma Virus-Based, Self-Replicating, Episomal Expression Vectors. The episomal expression vectors of the disclosure also may be based on replication functions of the papilloma family of virus, including but not limited to Bovine Papilloma Virus (BPV) and Human Papilloma Viruses (HPVs). BPV and HPVs persist as stably maintained plasmids in mammalian cells. —S trans-acting factors encoded by BPV and HPVs, namely E1 and E2, have also been identified which are necessary and sufficient for mediate replication in many cell types via minimal origin of replication (Ustav et al., EMBO J. 10:449-457 (1991); Ustavet al., EMBO J. 10:4231-4329, (1991); Ustav et al., Proc. Natl. Acad. Sci. USA 90:898-902 (1993)).

An episomal vector useful according to the disclosure is the BPV-I vector system described in Piirsoo et al., EMBO J., 15:1 (1996) and in WO 94/12629. The BPV-1 vector system described in Piirsoo et al. comprises a plasmid harboring the BPV-1 origin of replication (minimal origin plus extrachro osomal maintenance element) and optionally the E1 and E2 genes. The BPV-1 E1 and E2 genes are required for stable maintenance of a BPV episomal vector. These factors ensure that the plasmid is replicated to a stable copy number of up to thirty copies per cell independent of cell cycle status. The gene construct therefore persists stably in both dividing and non-dividing cells. This allows the maintenance of the gene construct in cells such as hemopoietic stem cells and more committed precursor cells.

The BPV origin of replication has been located at the 31 end of the upstream regulatory region within a 60-base pair (bp) DNA fragment (nucleotides (nt) 7914-7927) which includes binding sites for the E1 and E2 replication factors. The minimal origin of replication of HPV has also been characterized and located in the URR fragment (nt 7022-7927) of HPV (see, for example, Chiang et al., Proc. Natl. Acad. Sci. USA 89:5799-5803 (1992)). As used herein, “E1” refers to the protein encoded by nucleotides (nt) 849-2663 of BPV subtype 1 or by nt 832-2779 of HPV of subtype 11, to equivalent E1 proteins of other papilloma viruses, or to functional fragments or mutants of a papilloma virus E1 protein, e.g., fragments or mutants of E1 which possess the replicating properties of E1.

As used herein, “E2H refers to the protein encoded by nt 2594-3837 of BPV subtype 1 or by nt 2723-3823 of HPV subtype 11, to equivalent E2 proteins of other papilloma viruses, or to functional fragments or mutants of a papilloma virus E2 protein, e.g., fragments or mutants of E2 which possess the replicating properties of E2. “Minichromosomal maintenance element” (MME) refers to the extrachromosomal maintenance element of the papilloma viral genome to which viral or human proteins essential for papilloma viral replication bind, which region is essential for stable episomal maintenance of the papilloma viral MO in a host cell, as described in Piirsoo et al. (supra). Preferably, the MME is a sequence containing multiple binding sites for the transcriptional activator E2. The MME in BPV is herein defined as the region of BPV located within the upstream regulatory region which includes a minimum of about six sequential E2 binding sites, and which gives optimum stable maintenance with about ten sequential E2 binding sites. E2 binding site 9 is an example sequence for this site, as described hereinbelow, wherein the sequential sites are separated by a spacer of about 4-10 nucleotides, and optimally 6 nucleotides. E1 and E2 can be provided to the plasmid either in cis or in trans, also as described in WO 94/12629 and in Piirsoo et al. (supra).

“E2 binding site” refers to the minimum sequence of papillomavirus double-stranded DNA to which the E2 protein binds. An E2 binding site may include the sequence 5*ACCGTTGCCGGT 3′ (SEQ ID NO: 1098), which is high affinity E2 binding site 9 of the BPV-1 URR; alternatively, an E2 binding site may include permutations of binding site 9, which permutations are found within the URR, and fall within the generic E2 binding sequence 5′ ACCN6GGT 3′. Transcriptional activator E2 binding sites are, in most papillomaviruses, located in the upstream regulatory region, as in BPV and HPV. A vector which also is useful according to the disclosure may include a region of BPV between 6959-7945/1-470 on the BPV genetic map (as described in WO 94/12629), which region includes an origin of replication, a first promoter operatively associated with a gene of interest, the BPV E1 gene operatively associated with a second promoter to drive transcription of the E1 gene; and the BPV E2 gene operatively associated with a third promoter to drive transcription of the E2 gene.

E1 and E2 from BPV will replicate vectors containing the BPV origin or the origin of many HPV subtypes (Chiang et al., supra). E1 and E2 from HPV will replicate vectors via the BPV origin and via the origin of many HPV subtypes (Chiang et al., supra). As with all vectors of the disclosure, the BPV-based episomal expression vectors of the disclosure must persist through 2-5 or more divisions of the host cell.

See also U.S. Pat. No. 7,790,446 and Abroi et al. (2004) “Analysis of chromatin attachment and partitioning functions of bovine papillomavirus type 1 E2 protein. Journal of Virology 78:2100-13 which have shown that the BPV1 E2 protein dependent MME and EBV EBNA1 dependent FR segregation/partitioning activities function independently from replication of the plasmids. The stable-maintenance function of EBNA1/FR and E2/MME can be used to ensure long-time episomal maintenance for cellular replication origins.

Papovavirus-Based, Self-Replicating, Episomal Expression Vectors. The vectors of the disclosure also may be derived from a human papovavirus BK genomic DNA molecule. For example, the BK viral genome can be digested with restriction enzymes EcoRI and BamHI to produce a 5 kilobase (kb) fragment that contains the BK viral origin of replication sequences that can confer stable maintenance on vectors (see, for example, De Benedetti and Rhoads, Nucleic Acids Res. 19:1925 (1991), as can a 3.2 kb fragment of the BK virus (Cooper and Miron, Human Gene Therapy 4:557 (1993)).

The encoded AFFIMER® constructs of the present disclosure can be provided as circular or linear nucleic acids. The circular and linear nucleic acids are capable of directing expression of the AFFIMER® agent coding sequence in an appropriate subject cell. The at least one nucleic acid system for expressing an AFFIMER® agent may be chimeric, meaning that at least one of its components is heterologous with respect to at least one of its other components.

### A. Viral Vectors

Exemplary viral gene therapy system that are readily adapted for use in the present disclosure include plasmid, adenovirus, adeno-associated virus (AAV), retrovirus, lentivirus, herpes simplex virus, vaccinia virus, poxvirus, reovirus, measles virus, Semliki Forest virus, and the like. Preferred viral vectors are based on non-cytopathic eukaryotic viruses in which non-essential genes have been replaced with the nucleic acid construct carrying the nucleic acid sequences encoding the epitopes and targeting sequences of interest.

To further illustrate, encoded AFFIMER® constructs can be delivered in vivo using adenoviruses and adeno-associated (AAV) viruses, which are double-stranded DNA viruses that have already been approved for human use in gene therapy.

### 1. Adenovirus Vectors

One illustrative method for in vivo delivery of at least one nucleic acid sequence involves the use of an adenovirus (“AdV”) expression vector. AdVs are non-enveloped, double-stranded DNA viruses that neither integrate in the host genome nor replicate during cell division. AdV-mediated antibody gene transfer has shown therapeutic efficacy in a variety of different disease models advancing towards the clinic. Systemic mAb expression has mostly been pursued, via s.c. and especially i.v. and intramuscular AdV injection. See Wold et al. (2013) “Adenovirus vectors for gene therapy, vaccination and cancer gene therapy” Curr Gene Ther. 13 (6): 421-33; and Deal et al. “Engineering humoral immunity as prophylaxis or therapy” 2015 Curr Opin Immunol. 35:113-22. Other routes of delivery have focused on more local mAb production, such as via intranasal, intratracheal or intrapleural administration of the encoding AdV. The use of AdVs as oncolytic vectors is a popular approach particularly for generation of encoded antibodies at the site of tumors. Foreign genes delivered by current adenoviral gene delivery system are episomal, and therefore, have low genotoxicity to host cells. Therefore, gene therapy using adenoviral gene delivery systems may be considerably safe. The present disclosure specifically contemplates the delivery of AFFIMER® agents by expression of encoded AFFIMER® constructs delivered in the form of an adenoviral vector and delivery system.

Adenovirus has been usually employed as a gene delivery vector because of its mid-sized genome, ease of manipulation, high titer, wide target-cell range and high infectivity. Both ends of the viral genome contains 100-200 bp ITRs (inverted terminal repeats), which are cis elements necessary for viral DNA replication and packaging. The E1 region (E1A and E1B) of genome encodes proteins responsible for the regulation of transcription of the viral genome and a few cellular genes. The E2 region (E2A and E2B) encodes proteins responsible for viral DNA replication. Of adenoviral vectors developed so far, the replication incompetent adenovirus having the deleted E1 region is usually used and represent one exemplary choice of AdV for generating the encoded AFFIMER® constructs of the present disclosure. The deleted E3 region in adenoviral vectors may provide an insertion site for transgenes (Thimmappaya, B. et al., Cell, 31:543-551 (1982); and Riordan, J. R. et al., Science, 245:1066-1073 (1989)).

An “adenovirus expression vector” is meant to include those constructs containing adenovirus sequences sufficient to (a) support packaging of the construct and (b) to express a polynucleotide that encodes a polypeptide including an AFFIMER® agent such as a PD-L1 AFFIMER® polypeptide (the encoded AFFIMER® construct sequence). In some embodiments, the sequence for an encoded AFFIMER® construct may be inserted into the DA promoter region. According to an exemplary embodiment, the recombinant adenovirus comprises deleted E1B and E3 region and the nucleotide sequence for an encoded AFFIMER® construct is inserted into the deleted E1B and E3 region.

### 2. Adeno-Associated Virus Vectors (AAV)

AAVs (or “rAAV” for recombinant AAV) are non-enveloped small, single-stranded DNA viruses capable of infecting both dividing and non-dividing cells. Similar to AdV, AAV-based vectors remain in an episomal state in the nucleus and display a limited risk of integration. In contrast to the generally limited durability of AdV-mediated gene transfer, transgene expression can persist for years following intramuscular recombinant AAV (rAAV) vector delivery.

Alipogene tiparvovec (Glybera™), an rAAV encoding the human lipoprotein lipase gene, was approved in 2012 as the first gene therapy product in Europe. Since then, various rAAV-based gene therapy products are currently under clinical evaluation. In the context of antibody gene transfer, a variety of reports have demonstrated in vivo production of an anti-human immune deficiency virus (HIV) mAb in mice following intramuscular injection of the mAb-encoding rAAV. The rAAV vector's potential for combination therapy has also been demonstrated, e.g., by expressing two mAbs. Similar to AdV, intramuscular and i.v. rAAV administration have been most often pursued. Reviewed in Deal et al. “Engineering humoral immunity as prophylaxis or therapy” 2015 Curr Opin Immunol. 35:113-22. A variety of additional delivery sites have also been demonstrated to achieve more local therapeutic effects, including intracranial, intranasal, intravitreal, intrathecal, intrapleural, and intraperitoneal routes. With the utility of rAAV demonstrated for antibody gene transfer, the present disclosure also specifically contemplates the use of rAAV systems for the delivery of encoded AFFIMER® construct sequences in vivo and the production of AFFIMER® agents in the body of a patient as a consequence to expression of the rAAV construct.

One important feature to AAV is that these gene transfer viruses are capable of infecting non-dividing cells and various types of cells, making them useful in constructing the encoded AFFIMER® construct delivery system of this disclosure. The detailed descriptions for use and preparation of exemplary AAV vectors are found in, for example, U.S. Pat. Nos. 5,139,941 and 4,797,368, as well as LaFace et al, Viology, 162: 483486 (1988), Zhou et al., Exp. Hematol. (NY), 21: 928-933 (1993), Walsh et al, J. Clin. Invest., 94: 1440-1448 (1994) and Flotte et al., Gene Therapy, 2: 29-37 (1995). AAV is a good choice of delivery vehicles due to its safety, e.g., genetically engineered (recombinant) does not integrate into the host genome. Likewise, AAV is not pathogenic and not associated with any disease. The removal of viral coding sequences minimizes immune reactions to viral gene expression, and therefore, recombinant AAV does not evoke an inflammatory response.

Typically, a recombinant AAV virus is made by co-transfecting a plasmid containing the gene of interest (e.g., the coding sequence for an AFFIMER® agent) flanked by the two AAV terminal repeats (McLaughlin et al., J. Virol., 62:1963-1973 (1988); Samulski et al., J. Virol., 63:3822-3828 (1989)) and an expression plasmid containing the wild type AAV coding sequences without the terminal repeats (McCarty et al., J. Virol., 65:2936-2945 (1991)). Typically, viral vectors containing an encoded AFFIMER® construct are assembled from polynucleotides encoding the AFFIMER® polypeptide, suitable regulatory elements and elements necessary for expression of the encoded AFFIMER® construct which mediate cell transduction. In some embodiments, adeno-associated viral (AAV) vectors are employed. In a more specific embodiment, the AAV vector is an AAV1, AAV6, or AAV8.

The AAV expression vector which harbors the encoded AFFIMER® construct sequence bounded by AAV ITRs, can be constructed by directly inserting the selected sequence(s) into an AAV genome which has had the major AAV open reading frames (“ORFs”) excised therefrom.

For eukaryotic cells, expression control sequences typically include a promoter, an enhancer, such as one derived from an immunoglobulin gene, SV40, cytomegalovirus, etc. (see above), and a polyadenylation sequence which may include splice donor and acceptor sites. The polyadenylation sequence generally is inserted following the transgene sequences and before the 3′ ITR sequence.

Selection of these and other common vector and regulatory elements are conventional, and many such sequences are available. See, e.g., Sambrook et al., and references cited therein at, for example, pages 3.18-3.26 and 16.17-16.27 and Ausubel et al., Current Protocols in Molecular Biology, John Wiley & Sons, New York, 1989). Of course, not all vectors and expression control sequences will function equally well to express all of the transgenes of this disclosure. However, one of skill in the art may make a selection among these expression control sequences without departing from the scope of this disclosure. Suitable promoter/enhancer sequences may be selected by one of skill in the art using the guidance provided by this application. Such selection is a routine matter and is not a limitation of the molecule or construct.

### 3. Retrovirus Vectors

Non-cytopathic viruses useful in the context of delivery of encoded AFFIMER® constructs include retroviruses, the life cycle of which involves reverse transcription of genomic viral RNA into DNA with subsequent proviral integration into host cellular DNA. Retroviruses have been approved for human gene therapy trials. Most useful are those retroviruses that are replication-deficient (e.g., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are known to those of skill in the art.

In order to construct a retroviral vector, the AFFIMER® agent coding sequence is inserted into the viral genome in the place of certain viral sequences to produce a replication-defective virus. To produce virions, a packaging cell line containing the gag, pol and env genes but without the LTR (long terminal repeat) and psi (□) components is constructed (Mann et al., Cell, 33:153-159 (1983)). When a recombinant plasmid containing the cytokine gene, LTR and psi is introduced into this cell line, the psi sequence allows the RNA transcript of the recombinant plasmid to be packaged into viral particles, which are then secreted into the culture media (Nicolas and Rubinstein “Retroviral vectors,” In: Vectors: A survey of molecular cloning vectors and their uses, Rodriguez and Denhardt (eds.), Stoneham: Butterworth, 494-513 (1988)). The media containing the recombinant retroviruses is then collected, optionally concentrated and used for gene delivery system.

Successful gene transfer using such second-generation retroviral vectors has been reported. Kasahara et al. (Science, 266:1373-1376 (1994)) prepared variants of moloney murine leukemia virus in which the EPO (erythropoietin) sequence is inserted in the place of the envelope region, consequently, producing chimeric proteins having novel binding properties. Likely, the present gene delivery system can be constructed in accordance with the construction strategies for the second-generation retroviral vector.

In some embodiments, the retrovirus is a “gammaretroviruses”, which refers to a genus of the retroviridae family. Exemplary gammaretroviruses include mouse stem cell virus, murine leukemia virus, feline leukemia virus, feline sarcoma virus, and avian reticuloendotheliosis viruses.

In some embodiments, the retroviral vector for use in the present disclosure is a lentiviral vector, which refers to a genus of retroviruses that are capable of infecting dividing and non-dividing cells and typically produce high viral titers. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

Another class of widely used retroviral vectors that can be used for the delivery and expression of an encoded AFFIMER® construct include those based upon murine leukemia virus (MuLV), gibbon ape leukemia virus (GaLV) and combinations thereof (see, e.g., Buchscher et al., J. Virol. 66:2731-2739, 1992; Johann et al., J. Virol. 66:1635-1640, 1992; Sommerfelt et al., Virol. 176:58-59, 1990; Wilson et al., J. Virol. 63:2374-2378, 1989; Miller et al., J. Virol. 65:2220-2224, 1991; and PCT/US94/05700).

Still other retroviral vectors that can be also be used in the present disclosure include, e.g., vectors based on human foamy virus (HFV) or other viruses in the Spumavirus genera. Foamy viruses (FVes) are the largest retroviruses known today and are widespread among different mammals, including all non-human primate species, however, are absent in humans. This complete apathogenicity qualifies FV vectors as ideal gene transfer vehicles for genetic therapies in humans and clearly distinguishes FV vectors as gene delivery system from HIV-derived and also gammaretrovirus-derived vectors.

Suitable retroviral vectors for use herein are described, for example, in U.S. Pat. Nos. 5,399,346 and 5,252,479; and in WIPO publications WO 92/07573, WO 90/06997, WO 89/05345, WO 92/05266 and WO 92/14829, which provide a description of methods for efficiently introducing nucleic acids into human cells using such retroviral vectors. Other retroviral vectors include, for example, mouse mammary tumor virus vectors (e.g., Shackleford et al., Proc. Natl. Acad. Sci. U.S.A. 85:9655-9659, 1998), lentiviruses, and the like.

Additional retroviral viral delivery systems that can be readily adapted for delivery of a transgene encoding a PD-L1 AFFIMER® agent include, merely to illustrate Published PCT Applications WO/2010/045002, WO/2010/148203, WO/2011/126864, WO/2012/058673, WO/2014/066700, WO/2015/021077, WO/2015/148683, WO/2017/040815—the specifications and FIGS. of each of which are incorporated by reference herein.

In some embodiments, a retroviral vector contains all of the cis-acting sequences necessary for the packaging and integration of the viral genome, e.g., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail regarding retroviral vectors can be found in Boesen, et al., 1994, Biotherapy 6:291-302; Clowes, et ai, 1994, J. Clin. Invest. 93:644-651; Kiem, et al., 1994, Blood 83:1467-1473; Salmons and Gunzberg, 1993, Human Gene Therapy 4:129-141; Miller, et al., 1993, Meth. Enzymol. 217:581-599; and Grossman and Wilson, 1993, Curr. Opin. in Genetics and Devel. 3:110-114.

In some embodiments, the retrovirus is a recombinant replication competent retrovirus comprising: a nucleic acid sequence encoding a retroviral GAG protein; a nucleic acid sequence encoding a retroviral POL protein; a nucleic acid sequence encoding a retroviral envelope; an oncoretroviral polynucleotide sequence comprising Long-Terminal Repeat (LTR) sequences at the 5′ and 3′ end of the oncoretroviral polynucleotide sequence; a cassette comprising an internal ribosome entry site (IRES) operably linked to a coding sequence for an AFFIMER® agent, such as for a PD-L1 AFFIMER® agent, wherein the cassette is positioned 5′ to the U3 region of the 3′ LTR and 3′ to the sequence encoding the retroviral envelope; and cis-acting sequences for reverse transcription, packaging and integration in a target cell.

In some embodiments, the retrovirus is a recombinant replication competent retrovirus comprising: a retroviral GAG protein; a retroviral POL protein; a retroviral envelope; a retroviral polynucleotide comprising Long-Terminal Repeat (LTR) sequences at the 3′ end of the retroviral polynucleotide sequence, a promoter sequence at the 5′ end of the retroviral polynucleotide, the promoter being suitable for expression in a mammalian cell, a gag nucleic acid domain, a pol nucleic acid domain and an env nucleic acid domain; a cassette comprising an encoded AFFIMER® construct sequence, wherein the cassette is positioned 5′ to the 3′ LTR and is operably linked and 3′ to the env nucleic acid domain encoding the retroviral envelope; and cis-acting sequences necessary for reverse transcription, packaging and integration in a target cell.

In some embodiments of the recombinant replication competent retrovirus, the envelope is chosen from one of amphotropic, polytropic, xenotropic, 10A1, GALV, Baboon endogenous virus, RD114, rhabdovirus, alphavirus, measles or influenza virus envelopes.

In some embodiments of the recombinant replication competent retrovirus, the retroviral polynucleotide sequence is engineered from a virus selected from the group consisting of murine leukemia virus (MLV), Moloney murine leukemia virus (MoMLV), Feline leukemia virus (FeLV), Baboon endogenous retrovirus (BEV), porcine endogenous virus (PERV), the cat derived retrovirus RD114, squirrel monkey retrovirus, Xenotropic murine leukemia virus-related virus (XMRV), avian reticuloendotheliosis virus (REV), or Gibbon ape leukemia virus (GALV).

In some embodiments of the recombinant replication competent retrovirus, retrovirus is a gammaretrovirus.

In some embodiments of the recombinant replication competent retrovirus, there is a second cassette comprising a coding sequence for a second therapeutic protein, such as another checkpoint inhibitor polypeptide, a co-stimulatory polypeptide and/or a immunostimulatory cytokine (merely as examples), e.g., downstream of the cassette. In certain instances, the second cassette can include an internal ribosome entry site (IRES) or a minipromoter or a polIII promoter operably linked to the coding sequence for the second therapeutic protein.

In some embodiments of the recombinant replication competent retrovirus, it is a nonlytic, amphotropic retroviral replicating vector which, preferably, selectively infects and replicates in the cells of the tumor microenvironment.

### 4. Other Viral Vectors as Expression Constructs

In the context of vectored intratumoral encoded AFFIMER® construct gene transfer, oncolytic viruses have a distinct advantage, as they can specifically target tumor cells, boost therapeutic AFFIMER® agent expression, and amplify antitumor therapeutic responses. Oncolytic viruses, which overlap with certain viral systems described above, promote anti-tumor responses through selective tumor cell killing and induction of systemic anti-tumor immunity. The mechanisms of action are not fully elucidated but are likely to depend on viral replication within transformed cells, induction of primary cell death, interaction with tumor cell anti-viral elements and initiation of innate and adaptive anti-tumor immunity. Reviewed in Kaufman et al. 2015 “Oncolytic viruses: a new class of immunotherapy drugs” Nat Rev Drug Discov. 14 (9): 642-62. Many of the oncolytic viruses that are currently in the clinic have a natural tropism for cell surface proteins that are aberrantly expressed by cancer cells. To date, AdV, poxviruses, coxsackieviruses, poliovirus, measles virus, Newcastle disease virus, reovirus, and others have entered into early-phase clinical trials. In 2015, the FDA and EMA approved talimogene laherparepvec (T-VEC, Imlygic™), an oncolytic herpes virus armed with the gene for granulocyte-macrophage colony-stimulating factor (GM-CSF). The self-perpetuating nature of oncolytic viruses makes them an appealing platform for encoded AFFIMER® construct gene transfer of the present disclosure, as transgene products can be amplified along with viral replication, thereby maximizing therapeutic effect. Liu et al. 2008 “Oncolytic adenoviruses for cancer gene therapy” Methods Mol Biol. 433:243-58.

In the case of AFFIMER® agents that are large fusion proteins, e.g., which comprise other protein domains beyond a single AFFIMER® domain, local intratumoral expression can present an appealing strategy to overcome poor penetration in solid tumors if and where that might be an issue. Beckman et al. (2007) “Antibody constructs in cancer therapy: protein engineering strategies to improve exposure in solid tumors” Cancer 109 (2): 170-9; and Dronca et al. 2015 “Immunomodulatory antibody therapy of cancer: the closer, the better” Clin Cancer Res. 21 (5): 944-6. Likewise, intratumoral delivery of the encoded AFFIMER® construct and concomitant local expression of the AFFIMER® agent can create a better therapeutic index where dose-limiting toxicities might otherwise prevent reaching the effective intratumoral concentration for efficacy when the AFFIMER® agent is delivered (or expressed) systemically.

In the case of the PD-L1 AFFIMER® agents of the present disclosure, the immunomodulatory nature of these AFFIMER® agents are very relevant to the use of oncolytic viruses. Indeed, for oncolytic virus therapy, it is desirable to override immune checkpoint inhibitor networks and thereby create a pro-inflammatory environment within the cancer. Numerous clinical trials are currently underway to evaluate the combination of oncolytic viruses and conventional immunomodulatory mAb administration. Kaufman et al. 2015 “Oncolytic viruses: a new class of immunotherapy drugs” Nat Rev Drug Discov. 14 (9): 642-62; and Lichty et al. 2014 “Going viral with cancer immunotherapy” Nat Rev Cancer. 14 (8): 559-67. However, systemic treatment with checkpoint-blocking mAbs can lead to severe immune-related adverse effects, which may also be an issue for some embodiments of the subject PD-L1 AFFIMER® agents, highlighting the opportunity for local therapies, e.g., via encoded AFFIMER® construct-armed oncolytic viruses. Different studies have pursued this approach and can be readily adapted for use with the encoded AFFIMER® construct. Dias et al. armed a replication-deficient and -competent oncolytic AdV with an anti-human CTLA-4 mAb. Dias et al. 2012 “Targeted cancer immunotherapy with oncolytic adenovirus coding for a fully human monoclonal antibody specific for CTLA-4” Gene Ther. 19 (10): 988-98. Another system recently described (and that can be adapted for use with the encoded AFFIMER® construct of the present disclosure) involved armed oncolytic vaccinia viruses with anti-murine programmed cell death protein 1 (PD-1) Fab, scFv or full-length mAb. Reflecting virus replication, mAb levels in the tumor peaked 3-5 days after intratumoral injection at 9 or 30 μg/ml, depending on the tumor model. Serum mAb levels followed the same trend, albeit threefold, or more, lower, although mAb detection was lost after 5 days. Intratumorally expressed mAbs lasted longer compared to intratumoral injection of anti-PD-1 mAb protein, with follow-up limited to 11 days after injection. Fab and scFv expression were not reported. Anti-tumor responses of the virus armed with either the anti-PD-1 scFv or mAb were superior to the unarmed virus and as effective as the combination of the unarmed virus and systemic anti-PD-1 mAb protein injections. Kleinpeter et al. 2016 “Vectorization in an oncolytic vaccinia virus of an antibody, a Fab and a scFv against programmed cell death-1 (PD-1) allows their intratumoral delivery and an improved tumor-growth inhibition” Oncoimmunology. 5 (10): e1220467 (online).

Other viral vectors may be employed as a gene delivery system in the present disclosure. Vectors derived from viruses such as vaccinia virus (Puhlmann M. et al., Human Gene Therapy, 10:649-657 (1999); Ridgeway, “Mammalian expression vectors,” In: Vectors: A survey of molecular cloning vectors and their uses. Rodriguez and Denhardt, eds. Stoneham: Butterworth, 467-492 (1988); Baichwal and Sugden, “Vectors for gene transfer derived from animal DNA viruses: Transient and stable expression of transferred genes,” In: Kucherlapati R, ed. Gene transfer. New York: Plenum Press, 117-148 (1986) and Coupar et al., Gene, 68:1-10 (1988)), lentivirus (Wang G. et al., J. Clin. Invest., 104 (11): R55-62 (1999)), herpes simplex virus (Chamber R., et al., Proc. Natl. Acad. Sci USA, 92:1411-1415 (1995)), poxvirus (GCE, NJL, Krupa M, Esteban M., The poxvirus vectors MVA and NYVAC as gene delivery systems for vaccination against infectious diseases and cancer Curr Gene Ther 8 (2): 97-120 (2008)), reovirus, measles virus, Semliki Forest virus, and polioviruses may be used in the present delivery systems for transferring the gene of interest into cells. They offer several attractive features for various mammalian cells. Also included are hepatitis B viruses.

### B. Non-Viral Vectors

In 1990, Wolff et al. showed how injection of naked plasmid DNA (pDNA) into the skeletal muscle of mice led to the local expression of the encoded protein, kick-starting the field of DNA-based therapeutics. See Wolff et al. 1990 “Direct gene transfer into mouse muscle in vivo” Science. 247 (4949 Pt 1): 1465-8. The use of “pDNA” for delivering encoded AFFIMER® construct of the present disclosure waives the need for a virus as biological vector and presents an appealing platform for encoded AFFIMER® construct gene transfer. Compared to viral vectors, pDNA is considered low-immunogenic (allowing e.g., repeated dosing), is cheaper to produce, ship, and store, and has a much longer shelf-life. After entry in the nucleus, pDNA remains in a non-replicating non-integrating episomal state and is lost during the breakdown of the nuclear envelope at mitosis. pDNA has no defined restrictions regarding the size of the transgene compared to viral vectors, and its modular nature allows for straightforward molecular cloning, making them easy to manipulate and design for therapeutic use. Hardee et al. 2017 “Advances in non-viral DNA vectors for gene therapy” Genes. 8 (2): 65. Plasmids are used in about 17% of the ongoing or completed gene therapy clinical trials and showed to be well-tolerated and safe.

The method of DNA administration can greatly impact transgene expression. In vivo DNA-mediated encoded AFFIMER® construct gene transfer can utilize such physical methods of transfection used for antibody gene transfer, such as electroporation or hydrodynamic injection. Electroporation presents the propagation of electrical fields within tissues, which induces a transient increase in cell membrane permeability. Electrotransfer of DNA is a multistep process, involving (i) electrophoretic migration of DNA towards the plasma membrane, (ii) DNA accumulation and interaction with the plasma membrane, and (iii) intracellular trafficking of the DNA to the nucleus, after which gene expression can commence. Heller L C. 2015 “Gene electrotransfer clinical trials” Adv Genet. 89:235-62. Intramuscular, intratumoral and intradermal administration have been evaluated in clinical trials and are also suitable target tissues for electroporation of encoded AFFIMER® constructs.

Hydrodynamic-based transfection utilizes the i.v. injection of high volumes of pDNA, driving DNA molecules out of the blood circulation and into tissue. Other potentially less invasive physical delivery methods include sonoporation and magnetofection. DNA uptake can also be improved by complexing the molecules with chemical delivery vehicles (e.g., cationic lipids or polymers and lipid nanoparticles). Such techniques can also be applied to in vivo DNA-mediated encoded AFFIMER® construct gene transfer.

In addition to the choice of delivery method, encoded AFFIMER® construct transgene expression can be improved by modifying the make-up of pDNA constructs. See, for example, Hardee et al. 2017 “Advances in non-viral DNA vectors for gene therapy” Genes 8 (2): 65; and Simcikova et al. 2015 “Towards effective non-viral gene delivery vector” Biotechnol Genet Eng Rev. 31 (1-2): 82-107. Conventional pDNA consists of a transcription unit and bacterial backbone. The transcription unit carries the encoded AFFIMER® construct sequence along with regulatory elements. The bacterial backbone includes elements like an antibiotic resistance gene, an origin of replication, unmethylated CpG motifs, and potentially cryptic expression signals. Some of these sequences are required for the production of plasmid DNA. However, in general, for therapeutic encoded AFFIMER® construct gene therapy the presence of a bacterial backbone will likely be counterproductive. However, there are a variety of different types of available minimal vectors that can be selected, including minicircle DNA (mcDNA) which already been used for antibody gene transfer and can be readily adapted for encoded AFFIMER® construct gene transfer. Minicircles are plasmid molecules devoid of bacterial sequences, generated via a process of recombination, restriction and/or purification. Simcikova et al. 2015 supra. Elimination of the bacterial backbone has shown higher transfection efficiency and prolonged transgene expression in a variety of tissues.

Also provided herein is a linear nucleic acid, or linear expression cassette (“LEC”), that is capable of being efficiently delivered to a subject via electroporation and expressing the encoded AFFIMER® construct sequence included therein. The LEC may be any linear DNA devoid of any phosphate backbone. The LEC may contain a promoter, an intron, a stop codon, and/or a polyadenylation signal. The expression of the encoded AFFIMER® construct coding sequence may be controlled by the promoter.

### 1. Plasmid Vectors

In some embodiments, the encoded AFFIMER® constructs are delivered as plasmid vectors. Plasmid vectors have been extensively described in the art and are well known to those of skill in the art. See e.g., Sambrook et al., 1989, cited above. In the last few years, plasmid vectors have been used as DNA vaccines for delivering antigen-encoding genes to cells in vivo. They are particularly advantageous for this because they reduced safety concerns relative to other vectors. These plasmids, however, having a promoter compatible with the host cell, can express a peptide epitope encoded by nucleic acid within the plasmid. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

To expand the application and efficiency of using plasmid DNA to deliver an encoded AFFIMER® construct to tissue in vivo, different approaches can be pursued based on principles producing higher mAb expression or overall efficacy in prior art reports. A first strategy simply relies on giving multiple or repeated pDNA doses. Kitaguchi et al. 2005 “Immune deficiency enhances expression of recombinant human antibody in mice after nonviral in vivo gene transfer” Int J Mol Med 16 (4): 683-8; and Yamazaki et al. 2011 “Passive immune-prophylaxis against influenza virus infection by the expression of neutralizing anti-hemagglutinin monoclonal antibodies from plasmids” Jpn J Infect Dis. 64 (1): 40-9. Another approach relates to the use of a delivery adjuvant. pDNA electrotransfer can be enhanced by pre-treating the muscle with hyaluronidase, an enzyme that transiently breaks down hyaluronic acid, decreasing the viscosity of the extracellular matrix and facilitating DNA diffusion. Yamazaki et al. 2011, supra; and McMahon et al. 2001 “Optimisation of electrotransfer of plasmid into skeletal muscle by pretreatment with hyaluronidase: increased expression with reduced muscle damage” Gene Ther. 8 (16): 1264-70. For antibody gene transfer, this led to an increase in mAb expression by approximately 3.5-fold, achieving plasma peak titers of 3.5 μg/ml with 30 μg pDNA, and can be adapted by one skilled in the art for encoded AFFIMER® construct gene transfer. Still another strategy focuses on antibody or cassette engineering. Following codon-, RNA- and leader sequence-optimization, peak serum mAb or Fab titers have been attained with intramuscular electrotransfer of ‘optimized’ pDNA. See, for example, Flingai et al. 2015 “Protection against dengue disease by synthetic nucleic acid antibody prophylaxis/immunotherapy” Sci Rep. 5: 12616.

The purpose of the plasmid is the efficient delivery of nucleic acid sequences to and expression of therapeutic AFFIMER® agents in a cell or tissue. In particular, the purpose of the plasmid may be to achieve high copy number, avoid potential causes of plasmid instability and provide a means for plasmid selection. As for expression, the nucleic acid cassette contains the necessary elements for expression of the encoded AFFIMER® construct within the cassette. Expression includes the efficient transcription of an inserted gene, nucleic acid sequence, or nucleic acid cassette with the plasmid. Thus, in some aspects, a plasmid is provided for expression of encoded AFFIMER® construct which includes an expression cassette comprising the coding sequence for the AFFIMER® agent; also referred to as a transcription unit. When a plasmid is placed in an environment suitable for epitope expression, the transcriptional unit will express the AFFIMER® agent and anything else encoded in the construct. The transcription unit includes a transcriptional control sequence, which is transcriptionally linked with a cellular immune response element coding sequence. Transcriptional control sequence may include promoter/enhancer sequences such as cytomegalovirus (CMV) promoter/enhancer sequences, such as described above. However, those skilled in the art will recognize that a variety of other promoter sequences suitable for expression in mammalian cells, including human patient cells, are known and can similarly be used in the constructs disclosed herein. The level of expression of the AFFIMER® agent will depend on the associated promoter and the presence and activation of an associated enhancer element.

In some embodiments, the encoded AFFIMER® construct sequence (encoding the desired AFFIMER® agent) can be cloned into an expression plasmid which contains the regulatory elements for transcription, translation, RNA stability and replication (e.g., including a transcriptional control sequence). Such expression plasmids are well known in the art and one of ordinary skill would be capable of designing an appropriate expression construct for producing a recombinant AFFIMER® agent in vivo.

### 2. Minicircle

Minicircle (mcDNA)-based antibody gene transfer can also be adapted for delivery of encoded AFFIMER® construct to tissues in vivo. Under certain circumstances, plasmid DNA used for non-viral gene delivery can cause unacceptable inflammatory responses. Where this happens, immunotoxic responses are largely due to the presence of unmethylated CpG motifs and their associated stimulatory sequences on plasmids following bacterial propagation of plasmid DNA. Simple methylation of DNA in vitro may be enough to reduce an inflammatory response but can result in reduced gene expression. The removal of CpG islands by cloning out, or elimination of non-essential sequences has been a successful technique for reducing inflammatory responses. Yew et al. 2000 “Reduced inflammatory response to plasmid DNA vectors by elimination and inhibition of immunostimulatory CpG motifs” Mol Ther 1 (3), 255-62.

Since bacterial DNA contains on average 4 times more CpG islands than mammalian DNA, a good solution is to eliminate entirely the bacterial control regions, such as the origin of replication and antibiotic resistance genes, from gene delivery vectors during the process of plasmid production. Thus, the “parent” plasmid is recombined into a “minicircle” which generally comprises the gene to be delivered (in this case, the encoded AFFIMER® construct coding sequence) and suitable control regions for its expression, and a miniplasmid which generally comprises the remainder of the parent plasmid.

Removal of bacterial sequences needs to be efficient, using the smallest possible excision site, whilst creating supercoiled DNA minicircles which consist solely of gene expression elements under appropriate—preferably mammalian—control regions. Some techniques for minicircle production use bacterial phage lambda (2) integrase mediated recombination to produce minicircle DNA. See, for example, Darquet, et al. 1997 Gene Ther 4 (12): 1341-9; Darquet et al. 1999 Gene Ther 6 (2): 209-18; and Kreiss, et al. 1998 Appl Micbiol Biotechnol 49 (5): 560-7).

Therefore, embodiments of nucleic acid constructs described herein may be processed in the form of minicircle DNA. Minicircle DNA pertains to small (2-4 kb) circular plasmid derivatives that have been freed from all prokaryotic vector parts. Since minicircle DNA vectors contain no bacterial DNA sequences, they are less likely to be perceived as foreign and destroyed. As a result, these vectors can be expressed for longer periods of time compared to certain conventional plasmids. The smaller size of minicircles also extends their cloning capacity and facilitates their delivery into cells. Kits for producing minicircle DNA are known in the art and are commercially available (System Biosciences, Inc., Palo Alto, Calif.). Information on minicircle DNA is provided in Dietz et al., Vector Engineering and Delivery Molecular Therapy (2013); 218, 1526-1535 and Hou et al., Molecular Therapy-Methods & Clinical Development, Article number: 14062 (2015) doi: 10.1038/mtm.2014.62. More information on Minicircles is provided in Chen Z Y, He C Y, Ehrhardt A, Kay M A. Mol Ther. 2003 September; 8 (3): 495-500 and Minicircle DNA vectors achieve sustained expression reflected by active chromatin and transcriptional level. Gracey Maniar L E, Maniar J M, Chen Z Y, Lu J, Fire A Z, Kay M A. Mol Ther. 2013 January; 21 (1): 131-8

As a nonlimiting example, a minicircle DNA vector may be produced as follows. An expression cassette, which comprises the encoded AFFIMER® construct coding sequence along with regulatory elements for its expression, is flanked by attachment sites for a recombinase. A sequence encoding the recombinase is located outside of the expression cassette and includes elements for inducible expression (such as, for example, an inducible promoter). Upon induction of recombinase expression, the vector DNA is recombined, resulting in two distinct circular DNA molecules. One of the circular DNA molecules is relatively small, forming a minicircle that comprises the expression cassette for the encoded AFFIMER® construct; this minicircle DNA vector is devoid of any bacterial DNA sequences. The second circular DNA sequence contains the remaining vector sequence, including the bacterial sequences and the sequence encoding the recombinase. The minicircle DNA containing the encoded AFFIMER® construct sequence can then be separately isolated and purified. In some embodiments, a minicircle DNA vector may be produced using plasmids similar to pBAD.ϕ.C31.hFIX and pBAD.ϕ.C31.RHB. See, e.g., Chen et al. (2003) Mol. Ther. 8:495-500.

Exemplary recombinases that may be used for creating a minicircle DNA vector include but are not limited to, Streptomyces bacteriophage ϕ31 integrase, Cre recombinase, and the 2 integrase/DNA topoisomerase IV complex. Each of these recombinases catalyzes recombination between distinct sites. For example, ϕ31 integrase catalyzes recombination between corresponding attP and attB sites, Cre recombinase catalyzes recombination between loxP sites, and the λ integrase/DNA topoisomerase IV complex catalyzes recombination between bacteriophage λ attP and attB sites. In some embodiments, such as, for example, with ϕ31 integrase or with λ integrase in the absence of the λ is protein, the recombinase mediates an irreversible reaction to yield a unique population of circular products and thus high yields. In other embodiments, such as, for example, with Cre recombinase or with A integrase in the presence of the λ protein, the recombinase mediates a reversible reaction to yield a mixture of circular products and thus lower yields. The reversible reaction by Cre recombinase can be manipulated by employing mutant loxP71 and loxP66 sites, which recombine with high efficiency to yield a functionally impaired P71/66 site on the minicircle molecule and a wild-type loxP site on the minicircle molecule, thereby shifting the equilibrium towards the production of the minicircle DNA product.

Published U.S. application No. 20170342424 also describes a system making use of a parent plasmid which is exposed to an enzyme which causes recombination at recombination sites, thereby forming a (i) minicircle including the encoded AFFIMER® construct sequence and (ii) a miniplasmid comprising the remainder of the parent plasmid. One recombination site is modified at the 5′ end such that its reaction with the enzyme is less efficient than the wild type site, and the other recombination site is modified at the 3′ end such that its reaction with the enzyme is less efficient than the wild type site, and the other recombination site is modified at the 3′ end such that its reaction with the enzyme is less efficient than the wild type site, both modified sites being located in the minicircle after recombination. This favors the formation of minicircle.

### C. RNA-Mediated Encoded AFFIMER® Construct Gene Transfer

Exemplary nucleic acids or polynucleotides for the encoded PD-L1 AFFIMER® constructs of the present disclosure include but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, a-LNA having an a-L-ribo configuration (a diastereomer of LNA), 2′-amino-LNA having a 2 ‘-amino functionalization, and 2’-amino-a-LNA having a 2′-amino functionalization), ethylene nucleic acids (ENA), cyclohexenyl nucleic acids (CeNA) or hybrids or combinations thereof.

mRNA presents an emerging platform for antibody gene transfer that can be adapted by those skilled in the art for delivery of encoded AFFIMER® constructs of the present disclosure. Although current results differ considerably, in certain instances the mRNA constructs appear to be able to rival viral vectors in terms of generated serum mAb titers. Levels were in therapeutically relevant ranges within hours after mRNA administration, a marked shift in speed compared to DNA. The use of lipid nanoparticles (LNP) for mRNA transfection, rather than the physical methods typically required for DNA, can provide significant advantages in some embodiments towards application range.

In their 1990 study, Wolff et al. (1990, supra) found that, in addition to pDNA, intramuscular injection of in vitro transcribed (IVT) mRNA also led to local expression of the encoded protein. mRNA was not pursued as actively as DNA at that time because of its low stability. Progress over the past years allowed mRNA to catch up with DNA and viral vectors as a tool for gene transfer. Reviewed in Sahin et al. (2014) “mRNA-based therapeutics: developing a new class of drugs” Nat Rev Drug Discov. 13 (10): 759-80. Conceptually, there are several differences with these expression platforms. mRNA does not need to enter into the nucleus to be functional. Once it reaches the cytoplasm, mRNA is translated instantly. mRNA-based therapeutics are expressed more transiently compared to DNA- or viral vector-mediated gene transfer, and do not pose the risk of insertional mutagenesis in the host genome. mRNA production is relatively simple and inexpensive. In terms of administration, mRNA uptake can be enhanced using electroporation. Broderick et al. 2017 “Enhanced delivery of DNA or RNA vaccines by electroporation” Methods Mol Biol. 2017; 1499:193-200. Most focus, however, has gone to non-physical transfection methods. Indeed, a variety of mRNA complexing formulations have been developed, including lipid nanoparticles (LNP), which have proven to be safe and very efficient mRNA carriers for administration in a variety of tissues and i.v. Pardi et al. 2015 “Expression kinetics of nucleoside-modified mRNA delivered in lipid nanoparticles to mice by various routes” J Control Release 217:345-51. In line with this progress, IVT mRNA has reached the stage of clinical evaluation.

Beissert et al. WO2017162266 “RNA Replicon for Versatile and Efficient Gene Expression” describes agents and methods suitable for efficient expression of AFFIMER® polypeptides of the present disclosure, such as suitable for immunotherapeutic treatment for the prevention and therapy of tumors. For instance, the AFFIMER® agent coding sequence can be provided as an RNA replicon comprising a 5′ replication recognition sequence such as from an alphavirus 5′ replication recognition sequence. In some embodiments, the RNA replicon comprises a (modified) 5′ replication recognition sequence and an open reading frame encoding the AFFIMER® agent, in particular located downstream from the 5′ replication recognition sequence such as that the 5′ replication recognition sequence and the open reading frame do not overlap, e.g., the 5′ replication recognition sequence does not contain a functional initiation codon and in some embodiments does not contain any initiation codon. Most preferably, the initiation codon of the open reading frame encoding the AFFIMER® agent is in the 5′->3′ direction of the RNA replicon.

In some embodiments, to prevent immune activation, modified nucleosides can be incorporated into the in vitro-transcribed mRNA. In some embodiments, the IVT RNA can be 5′ capped, such an m7GpppG-capped or m7G5′ppp5′G2′-O-Met-capped IVT. Efficient translation of the modified mRNA can be ensured by removing double-stranded RNA. Moreover, the 5′ and 3′ UTRs and the poly(A) tail can be optimized for improved intracellular stability and translational efficiency. See, for example, Stadler et al. (2017) Nature Medicine 23:815-817 and Kariko et al. WO/2017/036889 “Method for Reducing Immunogenicity of RNA”.

In some embodiments, the mRNA that encodes the PD-L1 AFFIMER® agent may include at least one chemical modification described herein. As a non-limiting example, the chemical modification may be 1-methylpseudouridine, 5-methylcytosine or 1-methylpseudouridine and 5-methylcytosine. In some embodiments, the chemical modification is a pseudouridine or a modified 5 nucleoside, wherein said modified nucleoside is m5C, m5U, m₆A, s2U, Ψ, or 2′-O-methyl-U. In some embodiments, linear polynucleotides encoding at least one PD-L1 AFFIMER® agent that are made using only in vitro transcription (IVT) enzymatic synthesis methods are referred to as “IVT polynucleotides.” Methods of making IVT polynucleotides are known in the art and are described in International Publication Nos. WO 2007/024708A2 and WO 2013/151666, the contents of which are incorporated herein by reference in their entirety.

In another embodiment, the polynucleotides that encode the PD-L1 AFFIMER® agent of the present disclosure have portions or regions which differ in size and/or chemical modification pattern, chemical modification position, chemical modification percent or chemical modification population and combinations of the foregoing are known as “chimeric polynucleotides.” A “chimera” according to the present disclosure is an entity having two or more incongruous or heterogeneous parts or regions. As used herein a “part” or “region” of a polynucleotide is defined as any portion of the polynucleotide which is less than the entire length of the polynucleotide. Such constructs are taught in for example International Publication No. WO2015/034928.

In yet another embodiment, the polynucleotides of the present disclosure that are circular are known as “circular polynucleotides” or “circP.” As used herein, “circular polynucleotides” or “circP” means a single stranded circular polynucleotide which acts substantially like, and has the properties of, an RNA. The term “circular” is also meant to encompass any secondary or tertiary configuration of the circP. Such constructs are taught in for example International Publication Nos. WO2015/034925 and WO2015/034928, the contents of each of which are incorporated herein by reference in their entirety.

Exemplary mRNA (and other polynucleotides) that can be used to encode PD-L1 AFFIMER® agents of the present disclosure include those which can be adapted from the specifications and FIGS. of, for example, International Publication No.s WO2017/049275, WO2016/118724, WO2016/118725, WO2016/011226, WO2015/196128, WO/2015/196130, WO/2015/196118, WO/2015/089511, and WO2015/105926 (the later titled “Polynucleotides for the In vivo Production of Antibodies”), each of which is incorporated by reference herein.

Electroporation, as described below, is one exemplary method for introducing mRNA or other polynucleotides into a cell.

Lipid-containing nanoparticle compositions have proven effective as transport vehicles into cells and/or intracellular compartments for a variety of RNAs (and related polynucleotides described herein). These compositions generally include at least one “cationic” and/or ionizable lipids, phospholipids including polyunsaturated lipids, structural lipids (e.g., sterols), and lipids containing polyethylene glycol (PEG lipids). Cationic and/or ionizable lipids include, for example, amine-containing lipids that can be readily protonated.

D. Other Methods of Delivery of Encoded AFFIMER® Construct into Target Cells

The introduction into host cell of the gene delivery system can be performed through various methods known to those skilled in the art.

Where the present gene delivery system is constructed on the basis of viral vector construction, delivery can be performed as conventional infection methods known in the art.

Physical methods to enhance delivery both viral and non-viral encoded AFFIMER® constructs include electroporation (Neumann, E. et al., EMBO J., 1: 841 (1982); and Tur-Kaspa et al., Mol. Cell Biol., 6:716-718 (1986)), gene bombardment (Yang et al., Proc. Natl. Acad. Sci., 87:9568-9572 (1990) where DNA is loaded onto (e.g., gold) particles and forced to achieve penetration of the DNA into the cells, sonoporation, magnetofection, hydrodynamic delivery and the like, all of which are known to those of skill in the art.

### 1. Electroporation

In the past several years, there has been a great advance in the plasmid DNA delivery technology that is utilized for in vivo production of proteins. This included codon optimization for expression in human cells, RNA optimization to improve mRNA stability as well as more efficient translation at the ribosomal level, the addition of specific leader sequences to enhance translation efficiency, the creation of synthetic inserts to further enhance production in vivo and the use of improved adaptive electroporation (EP) delivery protocols to improve in vivo delivery. EP assists in the delivery of plasmid DNA by generating an electrical field that allows the DNA to pass into the cell more efficiently. In vivo electroporation is a gene delivery technique that has been used successfully for efficient delivery of plasmid DNA to many different tissues. Kim et al. “Gene therapy using plasmid DNA-encoded anti-HER2 antibody for cancers that overexpress HER2” (2016) Cancer Gene Ther. 23 (10): 341-347 teaches a vector and electroporation system for intramuscular injection and in vivo electroporation of the plasmids that results in high and sustained antibody expression in sera; the plasmid and electroporation system of Kim et al. can be readily adapted for the in vivo delivery of a plasmid for expressing an encoded PD-L1 AFFIMER® construct of the present disclosure.

Accordingly, in certain some embodiments of the present disclosure, the encoded AFFIMER® construct is introduced into target cells via electroporation.

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate at least one of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (VGX Pharmaceuticals, Blue Bell, Pa.) or Elgen electroporator (Genetronics, San Diego, Calif.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not be limited as the elements can function as one device or as separate elements in communication with one another. The electroporation component may be capable of delivering the pulse of energy that produces the constant current in the desired tissue and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but in some embodiments is a real-time feedback or instantaneous (e.g., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the encoded AFFIMER® constructs of the present disclosure, include those described in U.S. Pat. Nos. 7,245,963; 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

In some embodiments, the electroporation is carried using a minimally invasive electroporation device (“MID”). The device may comprise a hollow needle, DNA cassette, and fluid delivery means, wherein the device is adapted to actuate the fluid delivery means in use so as to concurrently (for example, automatically) inject the encoded AFFIMER® construct into body tissue during insertion of the needle into the body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

A desired encoded AFFIMER® construct in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the nucleic acid into the tissue. For example, if the tissue to be treated is a mucosa, skin or muscle, the agent is projected towards the mucosal or skin surface with sufficient force to cause the agent to penetrate through the stratum corneum and into dermal layers, or into underlying tissue and muscle, respectively. Needle-free injectors are well suited to deliver encoded AFFIMER® constructs to all types of tissues, including into tumors (intratumoral delivery).

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present disclosure include tumor tissue, skin and other epithelial tissues, liver tissue and muscle tissue, merely as examples.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

Use of in vivo electroporation enhances plasmid DNA uptake in tumor tissue, resulting in expression within the tumor, and delivers plasmids to muscle tissue, resulting in systemic expression of secreted proteins, such as cytokines (see, e.g., U.S. Pat. No. 8,026,223). Additional exemplary techniques, vectors and devices for electroporating PD-L1 AFFIMER® agent transgenes into cells in vivo include PCT Publications WO/2017/106795, WO/2016/161201, WO/2016/154473, WO/2016/112359 and WO/2014/066655.

Typically, the electric fields needed for in vivo cell electroporation are generally similar in magnitude to the fields required for cells in vitro. In some embodiments, the magnitude of the electric field ranges from approximately, 10 V/cm to about 1500 V/cm, 300 V/cm to 1500 V/cm, or 1000 V/cm to 1500 V/cm. Alternatively, lower field strengths (from about 10 V/cm to 100 V/cm, and more preferably from about 25 V/cm to 75 V/cm) the pulse length is long. For example, when the nominal electric field is about 25-75 V/cm, if is preferred that the pulse length is about 10 msec.

The pulse length can be about 10 s to about 100 ms. There can be any desired number of pulses, typically one to 100 pulses per second. The delay between pulses sets can be any desired time, such as one second. The waveform, electric field strength and pulse duration may also depend upon the type of cells and the type of molecules that are to enter the cells via electroporation.

Also encompassed are electroporation devices incorporating electrochemical impedance spectroscopy (“EIS”). Such devices provide real-time information on in vivo, in particular, intratumoral electroporation efficiency, allowing for the optimization of conditions. Examples of electroporation devices incorporating EIS can be found, e.g., in WO2016/161201, which is hereby incorporated by reference.

Uptake of the encoded AFFIMER® constructs of the present disclosure may also be enhanced by plasma electroporation also termed avalanche transfection. Briefly, microsecond discharges create cavitation microbubbles at electrode surface. The mechanical force created by the collapsing microbubbles combined with the magnetic field serve to increase transport efficiency across the cell membrane as compared with the diffusion mediated transport associated with conventional electroporation. The technique of plasma electroporation is described in U.S. Pat. Nos. 7,923,251 and 8,283,171. This technique may also be employed in vivo for the transformation of cells. Chaiberg, et al (2006) Investigative Ophthalmology & Visual Science 47:4083-4090; Chaiberg, et al U.S. Pat. No. 8,101,169 Issued Jan. 24, 2012.

Other alternative electroporation technologies are also contemplated. In vivo nucleic acid delivery can also be performed using cold plasma. Plasma is one of the four fundamental states of matter, the others being solid, liquid, and gas. Plasma is an electrically neutral medium of unbound positive and negative particles (e.g., the overall charge of a plasma is roughly zero). A plasma can be created by heating a gas or subjecting it to a strong electromagnetic field, applied with a laser or microwave generator. This decreases or increases the number of electrons, creating positive or negative charged particles called ions (Luo, et al. (1998) Phys. Plasma 5:2868-2870) and is accompanied by the dissociation of molecular bonds, if present.

Cold plasmas (e.g., non-thermal plasmas) are produced by the delivery of pulsed high voltage signals to a suitable electrode. Cold plasma devices may take the form of a gas jet device or a dielectric barrier discharge (DBD) device. Cold temperature plasmas have attracted a great deal of enthusiasm and interest by virtue of their provision of plasmas at relatively low gas temperatures. The provision of plasmas at such a temperature is of interest to a variety of applications, including wound healing, anti-bacterial processes, various other medical therapies and sterilization. As noted earlier, cold plasmas (e.g., non-thermal plasmas) are produced by the delivery of pulsed high voltage signals to a suitable electrode. Cold plasma devices may take the form of a gas jet device, a dielectric barrier discharge (DBD) device or multi-frequency harmonic-rich power supply.

In some embodiments, the present disclosure provides a method for treating a subject having a tumor, the method comprising: injecting the tumor with an effective dose of plasmid coding for a PD-L1 AFFIMER® agent; and administering electroporation therapy to the tumor. In some embodiments, the electroporation therapy further comprises the administration of at least one voltage pulse of about 200 V/cm to about 1500 V/cm over a pulse width of about 100 microseconds to about 20 milliseconds.

In some embodiments, the plasmid (or a second electroporated plasmid) further encodes at least one immunostimulatory cytokine, such as selected from the group encoding IL-12, IL-15, and a combination of IL-12 and IL-15.

### 2. Transfection Enhancing Formulations

Encoded AFFIMER® constructs can also be encapsulated in liposomes, preferably cationic liposomes (Wong, T. K. et al., Gene, 10: 87 (1980); Nicolau and Sene, Biochim. Biophys. Acta, 721:185-190 (1982); and Nicolau et al., Methods Enzymol., 149:157-176 (1987)) or polymersomes (synthetic liposomes) which can interact with the cell membrane and fuse or undergo endocytosis to effect nucleic acid transfer into the cell. The DNA also can be formed into complexes with polymers (polyplexes) or with dendrimers which can directly release their load into the cytoplasm of a cell.

Illustrative carriers useful in this regard include microparticles of poly(lactide-co-glycolide), polyacrylate, latex, starch, cellulose, dextran and the like. Other illustrative carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active agent contained within a sustained release formulation depends upon the site of implantation, the rate and expected duration of release and the nature of the condition to be treated or prevented.

Biodegradable microspheres (e.g., polylactate polyglycolate) may be employed as carriers for compositions. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems such as described in WO/99 40934, and references cited therein, will also be useful for many applications. Another illustrative carrier/delivery system employs a carrier comprising particulate-protein complexes, such as those described in U.S. Pat. No. 5,928,647, which can have the added benefit when used intratumorally to deliver the coding sequence for a PD-L1 AFFIMER® polypeptide.

Biodegradable polymeric nanoparticles facilitate nonviral nucleic acid transfer to cells. Small (approximately 200 nm), positively charged (approximately 10 mV) particles are formed by the self-assembly of cationic, hydrolytically degradable poly(beta-amino esters) and plasmid DNA.

Polynucleotides may also be administered to cells by direct microinjection, temporary cell permeabilizations (e.g., co-administration of repressor and/or activator with a cell permeabilizing agent), fusion to membrane translocating peptides, and the like.

Lipid-mediated nucleic acid delivery and expression of foreign nucleic acids, including mRNA, in vitro and in vivo has been very successful. Lipid based non-viral formulations provide an alternative to viral gene therapies. Current in vivo lipid delivery methods use subcutaneous, intradermal, intratumoral, or intracranial injection. Advances in lipid formulations have improved the efficiency of gene transfer in vivo (see PCT Application WO 98/07408). For instance, a lipid formulation composed of an equimolar ratio of 1,2-bis(oleoyloxy)-3-(trimethyl ammonio) propane (DOTAP) and cholesterol can significantly enhance systemic in vivo gene transfer. The DOTAP:cholesterol lipid formulation forms unique structure termed a “sandwich liposome”. This formulation is reported to “sandwich” DNA between an invaginated bi-layer or ‘vase’ structure. Beneficial characteristics of these lipid structures include a positive p, colloidal stabilization by cholesterol, two-dimensional nucleic acid packing and increased serum stability.

Cationic liposome technology is based on the ability of amphipathic lipids, possessing a positively charged head group and a hydrophobic lipid tail, to bind to negatively charged DNA or RNA and form particles that generally enter cells by endocytosis. Some cationic liposomes also contain a neutral co-lipid, thought to enhance liposome uptake by mammalian cells. Similarly, other polycations, such as poly-l-lysine and polyethylene-imine, complex with nucleic acids via charge interaction and aid in the condensation of DNA or RNA into nanoparticles, which are then substrates for endosome-mediated uptake. Several of these cationic-nucleic acid complex technologies have been developed as potential clinical products, including complexes with plasmid DNA (pDNA), oligodeoxynucleotides, and various forms of synthetic RNA, and be used as part of the delivery system for the encoded AFFIMER® construct of the present disclosure.

The encoded AFFIMER® construct disclosed herein may be associated with polycationic molecules that serve to enhance uptake into cells. Complexing the nucleic acid construct with polycationic molecules also helps in packaging the construct such their size is reduced, which is believed to assist with cellular uptake. Once in the endosome, the complex dissociates due to the lower pH, and the polycationic molecules can disrupt the endosome's membrane to facilitate DNA escape into the cytoplasm before it can be degraded. Preliminary data shows that the nucleic acid construct embodiments had enhanced uptake into SCs over DCs when complexed with the polycationic molecules polylysine or polyethyleneimine.

One example of polycationic molecules useful for complexing with nucleic acid constructs includes cell penetrating peptides (CPP), examples include polylysine (described above), polyarginine and Tat peptides. Cell penetrating peptides (CPP) are small peptides which can bind to DNA and once released penetrate cell membranes to facilitate escape of the DNA from the endosome to the cytoplasm. Another example of a CPP pertains to a 27-residue chimeric peptide, termed MPG, was shown some time ago to bind ss- and ds-oligonucleotides in a stable manner, resulting in a non-covalent complex that protected the nucleic acids from degradation by DNase and effectively delivered oligonucleotides to cells in vitro (Mahapatro A, et al., J Nanobiotechnol, 2011, 9:55). The complex formed small particles of approximately 150 nm to 1 um when different peptide: DNA ratios were examined, and the 10:1 and 5:1 ratios (150 nm and 1 um respectively). Another CPP pertains to a modified tetrapeptide [tetralysine containing guanidinocarbonylpyrrole (GCP) groups (TL-GCP)], which was reported to bind with high affinity to a 6.2 kb plasmid DNA resulting in a positive charged aggregate of 700-900 nm Li et al., Agnew Chem Int Ed Enl 2015; 54 (10): 2941-4). RNA can also be complexed by such polycationic molecules for in vivo delivery.

Other examples of polycationic molecules that may be complexed with the nucleic acid constructs described herein include polycationic polymers commercially available as JETPRIME® and In vivo JET (Polypus-transfection, S.A., Illkirch, France).

In some embodiments, the present disclosure contemplates a method of delivering an mRNA (or other polynucleotide) f encoding a PD-L1 AFFIMER® agent to a patient's cells by administering a nanoparticle composition comprising (i) a lipid component, a phospholipid, a structural lipid, and a PEG lipid; and (ii) an mRNA (or other polynucleotide), said administering comprising contacting said mammalian cell with said nanoparticle composition, whereby said mRNA (or other polynucleotide) is delivered to said cell.

In exemplary embodiments, the PEG lipid is selected from the group consisting of a PEG-modified phosphatidylethanolamine, a PEG-modified phosphatide acid, a PEG-modified ceramide, a PEG-modified dialkylamine, a PEG-modified diacylglycerol and a PEG-modified dialkylglycerol. In exemplary embodiments, the structural lipid is selected from the group consisting of cholesterol, fecosterol, sitosterol, ergosterol, campesterol, stigmasterol, brassicasterol, tomatidine, ursolic acid, and alphatocopherol. In some embodiments, the structural lipid is cholesterol.

In exemplary embodiments, the phospholipid includes a moiety selected from the group consisting of phosphatidyl choline, phosphatidyl ethanolamine, phosphatidyl glycerol, phosphatidyl serine, phosphatidic acid, 2-lysophosphatidyl choline, and a sphingomyelin. In some embodiments, the phospholipid includes at least one fatty acid moiety selected from the group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanoic acid, eicosapentaenoic acid, behenic acid, docosapentaenoic acid, and docosahexaenoic acid. In some embodiments, the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (1 8:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanola mine (DOPE), 1,2-diphytanoyl-sn-glycero-3-phosphoethanolamine (ME 1 6.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin In some embodiments, the phospholipid is DOPE or DSPC.

To further illustrate, the phospholipid can be DOPE and said the component can comprise about 35 mol % to about 45 mol % said compound, about 10 mol % to about 20 mol % DOPE, about 38.5 mol % to about 48.5 mol % structural lipid, and about 1.5 mol % PEG lipid. The lipid component can be about 40 mol % said compound, about 15 mol % phospholipid, about 43.5 mol % structural lipid, and about 1.5 mol % PEG lipid.

In some embodiments, the wt/wt ratio of lipid component to PD-L1 AFFIMER® agent encoding mRNA (or other polynucleotide) is from about 5:1 to about 50:1, or about 10:1 to about 40:1

In some embodiments, the mean size of said nanoparticle composition is from about 50 nm to about 150 nm, or from about 80 nm to about 120 nm.

In some embodiments, the polydispersity index of said nanoparticle composition is from about 0 to about 0.18, or from about 0.13 to about 0.17.

In some embodiments, the nanoparticle composition has a zeta potential of about-10 to about +20 mV.

In some embodiments, the nanoparticle composition further comprises a cationic and/or ionizable lipid selected from the group consisting of 3-(didodecylamino)-N1,N 1,4-tridodecyl-1-piperazineethanamine (KL1 0), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), and (2R)-2-({8-[(3P)-cholest-5-en-3-yloxy]octyl}oxy)-N,N-dimethyl-3-[(9Z,12Z)-octadeca-9,12-dien-1-yl oxy]propan-1-amine (Octyl-CLinDMA (2R)).

For exemplary lipid nanoparticle compositions and other polymeric carrier compositions, see International Publication Nos. WO 2016/118724A1, WO 2017/112865A1, WO 2017/049245A2, and WO2012013326A1.

### V. Expression Methods and Systems

PD-L1 AFFIMER® agent proteins described herein can be produced by any suitable method known in the art. Such methods range from direct protein synthesis methods to constructing a DNA sequence encoding polypeptide sequences and expressing those sequences in a suitable host. For those recombinant AFFIMER® agent proteins including further modifications, such as a chemical modifications or conjugation, the recombinant AFFIMER® agent protein can be further manipulated chemically or enzymatically after isolation form the host cell or chemical synthesis.

The present disclosure includes recombinant methods and nucleic acids for recombinantly expressing the recombinant AFFIMER® agent proteins of the present disclosure comprising (i) introducing into a host cell a polynucleotide encoding the amino acid sequence of said AFFIMER® agent, for example, wherein the polynucleotide is in a vector and/or is operably linked to a promoter; (ii) culturing the host cell (e.g., eukaryotic or prokaryotic) under condition favorable to expression of the polynucleotide and, (iii) optionally, isolating the AFFIMER® agent from the host cell and/or medium in which the host cell is grown. See e.g., WO 04/041862, WO 2006/122786, WO 2008/020079, WO 2008/142164 or WO 2009/068627.

In some embodiments, a DNA sequence encoding a recombinant AFFIMER® agent protein of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once a nucleic acid sequence encoding a recombinant AFFIMER® agent protein of the disclosure has been obtained, the vector for the production of the recombinant AFFIMER® agent protein may be produced by recombinant DNA technology using techniques well known in the art. Methods which are well known to those skilled in the art can be used to construct expression vectors containing the recombinant AFFIMER® agent coding sequences and appropriate transcriptional and translational control signals. These methods include, for example, in vitro recombinant DNA techniques, synthetic techniques, and in vivo genetic recombination. (See, for example, the techniques described in Sambrook et al, 1990, MOLECULAR CLONING, A LABORATORY MANUAL, 2d Ed., Cold Spring Harbor Laboratory, Cold Spring Harbor, N.Y. and Ausubel et al. eds., 1998, CURRENT PROTOCOLS IN MOLECULAR BIOLOGY, John Wiley & Sons, NY).

An expression vector comprising the nucleotide sequence of a recombinant AFFIMER® agent protein can be transferred to a host cell by conventional techniques (e.g., electroporation, liposomal transfection, and calcium phosphate precipitation) and the transfected cells are then cultured by conventional techniques to produce the recombinant AFFIMER® agent protein of the disclosure. In specific embodiments, the expression of the recombinant AFFIMER® agent protein is regulated by a constitutive, an inducible or a tissue, specific promoter.

The expression vector may include an origin of replication, such as may be selected based upon the type of host cell being used for expression. By way of example, the origin of replication from the plasmid pBR322 (Product No. 303-3s, New England Biolabs, Beverly, Mass.) is useful for most Gram-negative bacteria while various origins from SV40, polyoma, adenovirus, vesicular stomatitus virus (VSV) or papillomaviruses (such as HPV or BPV) are useful for cloning vectors in mammalian cells. Generally, the origin of replication component is not needed for mammalian expression vectors (for example, the SV40 origin is often used because it contains the early promoter).

The vector may include at least one selectable marker gene, e.g., genetic elements that encode a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells, (b) complement auxotrophic deficiencies of the cell; or (c) supply critical nutrients not available from complex media. Preferred selectable markers are the kanamycin resistance gene, the ampicillin resistance gene, and the tetracycline resistance gene. A neomycin resistance gene may also be used for selection in prokaryotic and eukaryotic host cells. Other selection genes may be used to amplify the gene which will be expressed. Amplification is a process where genes which are in greater demand for the production of a protein critical for growth are reiterated in tandem within the chromosomes of successive generations of recombinant cells. Examples of selectable markers for mammalian cells include dihydrofolate reductase (DHFR) and thymidine kinase. The mammalian cell transformants are placed under selection pressure which only the transformants are uniquely adapted to survive by virtue of the marker present in the vector. Selection pressure is imposed by culturing the transformed cells under conditions in which the concentration of selection agent in the medium is successively changed, thereby leading to amplification of both the selection gene and the DNA that encodes the recombinant AFFIMER® agent protein. As a result, increased quantities of the recombinant AFFIMER® agent protein are synthesized from the amplified DNA.

The vector may also include at least one ribosome binding site, which will be transcribed into the mRNA including the coding sequence for the recombinant AFFIMER® agent protein. For example, such a site is characterized by a Shine-Dalgarno sequence (prokaryotes) or a Kozak sequence (eukaryotes). The element is typically located 3′ to the promoter and 5′ to the coding sequence of the polypeptide to be expressed. The Shine-Dalgarno sequence is varied but is typically a polypurine (having a high A-G content). Many Shine-Dalgarno sequences have been identified, each of which can be readily synthesized using methods set forth above and used in a prokaryotic vector.

The expression vectors will typically contain a promoter that is recognized by the host organism and operably linked to a nucleic acid molecule encoding the recombinant AFFIMER® agent protein. Either a native or heterologous promoter may be used depending on the host cell used for expression and the yield desired.

Promoters for use with prokaryotic hosts include the beta-lactamase and lactose promoter systems; alkaline phosphatase, a tryptophan (trp) promoter system; and hybrid promoters such as the tac promoter. Other known bacterial promoters are also suitable. Their sequences have been published, and they can be ligated to a desired nucleic acid sequence(s), using linkers or adapters as desired to supply restriction sites.

Promoters for use with yeast hosts are also known in the art. Yeast enhancers are advantageously used with yeast promoters. Suitable promoters for use with mammalian host cells are well known and include those obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40). Other suitable mammalian promoters include heterologous mammalian promoters, e.g., heat-shock promoters and the actin promoter.

Additional promoters which may be used for expressing the selective binding agents of the disclosure include but are not limited to: the SV40 early promoter region (Bernoist and Chambon, Nature, 290:304-310, 1981); the CMV promoter; the promoter contained in the 3′ long terminal repeat of Rous sarcoma virus (Yamamoto et al. (1980), Cell 22:787-97); the herpes thymidine kinase promoter (Wagner et al. (1981), Proc. Natl. Acad. Sci. U.S.A. 78:1444-5); the regulatory sequences of the metallothionine gene (Brinster et al, Nature, 296; 39-42, 1982); prokaryotic expression vectors such as the beta-lactamase promoter (Villa-Kamaroff, et al., Proc. Natl. Acad. Sci. U.S.A., 75; 3727-3731, 1978); or the tac promoter (DeBoer, et al. (1983), Proc. Natl. Acad. Sci. U.S.A., 80:21-5). Also of interest are the following animal transcriptional control regions, which exhibit tissue specificity and have been utilized in transgenic animals: the elastase I gene control region which is active in pancreatic acinar cells (Swift et al. (1984), Cell 38:639-46; Ornitz et al. (1986), Cold Spring Harbor Symp. Quant. Biol. 50:399-409; MacDonald (1987), Hepatology 7:425-515); the insulin gene control region which is active in pancreatic beta cells (Hanahan (1985), Nature 315:115-22); the immunoglobulin gene control region which is active in lymphoid cells (Grosschedl et al. (1984), Cell 38; 647-58; Adames et al. (1985), Nature 318; 533-8; Alexander et al. (1987), Mol. Cell. Biol. 7:1436-44); the mouse mammary tumor virus control region which is active in testicular, breast, lymphoid and mast cells (Leder et al. (1986), Cell 45:485-95), albumin gene control region which is active in liver (Pinkert et al. (1987), Genes and Devel. 1:268-76); the alphafetoprotein gene control region which is active in liver (Krumlauf et al. (1985), Mol. Cell. Biol. 5:1639-48; Hammer et al. (1987), Science, 235:53-8); the alpha 1-antitrypsin gene control region which is active in the liver (Kelsey et al. (1987), Genes and Devel. 1:161-71); the beta-globin gene control region which is active in myeloid cells (Mogram et al., Nature, 315 338-340, 1985; Kollias et al. (1986), Cell 46:89-94); the myelin basic protein gene control region which is active in oligodendrocyte cells in the brain (Readhead et al. (1987), Cell, 48:703-12); the myosin light chain-2 gene control region which is active in skeletal muscle (Sani (1985), Nature, 314:283-6); and the gonadotropic releasing hormone gene control region which is active in the hypothalamus (Mason et al. (1986), Science 234:1372-8).

An enhancer sequence may be inserted into the vector to increase transcription in eukaryotic host cells. Several enhancer sequences available from mammalian genes are known (e.g., globin, elastase, albumin, alpha-feto-protein and insulin). Typically, however, an enhancer from a virus will be used. The SV40 enhancer, the cytomegalovirus early promoter enhancer, the polyoma enhancer, and adenovirus enhancers are exemplary enhancing elements for the activation of eukaryotic promoters.

While an enhancer may be spliced into the vector at a position 5′ or 3′ to the polypeptide coding region, it is typically located at a site 5′ from the promoter.

Vectors for expressing nucleic acids include those which are compatible with bacterial, insect, and mammalian host cells. Such vectors include, inter alia, pCRII, pCR3, and pcDNA3.1 (Invitrogen Company, San Diego, Calif.), pBSII (Stratagene Company, La Jolla, Calif.), pET15 (Novagen, Madison, Wis.), pGEX (Pharmacia Biotech, Piscataway, N.J.), pEGFP-N2 (Clontech, Palo Alto, Calif.), pETL (BlueBacII; Invitrogen), pDSR-alpha (PCT Publication No. WO90/14363) and pFastBacDual (Gibco/BRL, Grand Island, N.Y.).

Additional possible vectors include but are not limited to, cosmids, plasmids or modified viruses, but the vector system must be compatible with the selected host cell. Such vectors include but are not limited to plasmids such as Bluescript® plasmid derivatives (a high copy number ColEl-based phagemid, Stratagene Cloning Systems Inc., La Jolla Calif.), PCR cloning plasmids designed for cloning Taq-amplified PCR products (e.g., TOPO™. TA Cloning® Kit, PCR2.1 plasmid derivatives, Invitrogen, Carlsbad, Calif.), and mammalian, yeast or virus vectors such as a baculovirus expression system (pBacPAK plasmid derivatives, Clontech, Palo Alto, Calif.). The recombinant molecules can be introduced into host cells via transformation, transfection, infection, electroporation, or other known techniques

Eukaryotic and prokaryotic host cells, including mammalian cells as hosts for expression of the recombinant AFFIMER® agent protein disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. Fungal cells include yeast and filamentous fungus cells including, for example, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., Yarrowia lipolytica, and Neurospora crassa.

A variety of host-expression vector systems may be utilized to express the recombinant AFFIMER® agent protein of the disclosure. Such host-expression systems represent vehicles by which the coding sequences of the recombinant AFFIMER® agent protein may be produced and subsequently purified, but also represent cells which may, when transformed or transfected with the appropriate nucleotide coding sequences, express the recombinant AFFIMER® agent protein of the disclosure in situ. These include but are not limited to, microorganisms such as bacteria (e.g., E. coli and B. subtilis) transformed with recombinant bacteriophage DNA, plasmid DNA or cosmid DNA expression vectors containing AFFIMER® agent protein coding sequences; yeast (e.g., Saccharomyces pichia) transformed with recombinant yeast expression vectors containing AFFIMER® agent protein coding sequences; insect cell systems infected with recombinant virus expression vectors (e.g., baculovirus) containing the AFFIMER® agent protein coding sequences; plant cell systems infected with recombinant virus expression vectors (e.g., cauliflower mosaic virus (CuMV) and tobacco mosaic virus (TMV)) or transformed with recombinant plasmid expression vectors (e.g., Ti plasmid) containing AFFIMER® agent protein coding sequences; or mammalian cell systems (e.g., COS, CHO, BHK, 293, 293T, 3T3 cells, lymphotic cells (see U.S. Pat. No. 5,807,715), Per C.6 cells (rat retinal cells developed by Crucell)) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the recombinant AFFIMER® agent protein being expressed. For example, when a large quantity of such a protein is to be produced, for the generation of pharmaceutical compositions of the recombinant AFFIMER® agent protein, vectors which direct the expression of high levels of fusion protein products that are readily purified may be desirable. Such vectors include but are not limited, to the E. coli expression vector pUR278 (Ruther et al. (1983) “Easy Identification Of cDNA Clones,” EMBO J. 2:1791-1794), in which the AFFIMER® agent protein coding sequence may be ligated individually into the vector in frame with the lac Z coding region so that a fusion protein is produced; pIN vectors (Inouye et al. (1985) “Up-Promoter Mutations In The Lpp Gene Of Escherichia coli,” Nucleic Acids Res. 13:3101-3110; Van Heeke et al. (1989) “Expression Of Human Asparagine Synthetase In Escherichia coli,” J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free gluta-thione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The AFFIMER® agent protein coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the AFFIMER® agent protein coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts. (see e.g., see Logan et al. (1984) “Adenovirus Tripartite Leader Sequence Enhances Translation Of mRNAs Late After Infection,” Proc. Natl. Acad. Sci. (U.S.A.) 81:3655-3659). Specific initiation signals may also be required for efficient translation of inserted AFFIMER® agent protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter et al. (1987) “Expression and Secretion Vectors For Yeast,” Methods in Enzymol. 153:516-544).

In addition, a host cell strain may be chosen which modulates the expression of the inserted sequences or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 293T, 3T3, WI38, BT483, Hs578T, HTB2, BT20 and T47D, CRL7030 and Hs578Bst.

For long-term, high-yield production of recombinant proteins, stable expression is contemplated. For example, cell lines which stably express an antibody of the disclosure may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the recombinant AFFIMER® agent proteins of the disclosure. Such engineered cell lines may be particularly useful in screening and evaluation of compounds that interact directly or indirectly with the recombinant AFFIMER® agent proteins.

A number of selection systems may be used, including but not limited to the herpes simplex virus thymidine kinase (Wigler et al. (1977) “Transfer of Purified Herpes Virus Thymidine Kinase Gene to Cultured Mouse Cells,” Cell 11:223-232), hypoxanthine-guanine phosphoribosyltransferase (Szybalska et al. (1962) “Genetics Of Human Cess Line. IV. DNA-Mediated Heritable Transformation of a Biochemical Trait,” Proc. Natl. Acad. Sci. (U.S.A.) 48:2026-2034), and adenine phosphoribosyltransferase (Lowy et al. (1980) “Isolation Of Transforming DNA: Cloning The Hamster Aprt Gene,” Cell 22:817-823) genes can be employed in tk-, hgprt- or aprt-cells, respectively. Also, antimetabolite resistance can be used as the basis of selection for the following genes: dhfr, which confers resistance to methotrexate (Wigler et al. (1980) “Transformation Of Mammalian Cells With An Amplfiable Dominant-Acting Gene,” Proc. Natl. Acad. Sci. (U.S.A.) 77:3567-3570; O'Hare et al. (1981) “Transformation Of Mouse Fibroblasts To Methotrexate Resistance By A Recombinant Plasmid Expressing A Prokaryotic Dihydrofolate Reductase,” Proc. Natl. Acad. Sci. (U.S.A.) 78:1527-1531); gpt, which confers resistance to mycophenolic acid (Mulligan et al. (1981) “Selection For Animal Cells That Express The Escherichia coli Gene Coding For Xanthine-Guanine Phosphoribosyltransferase,” Proc. Natl. Acad. Sci. (U.S.A.) 78:2072-2076); neo, which confers resistance to the aminoglycoside G-418 (Tachibana et al. (1991) “Altered Reactivity Of Immunoglobutin Produced By Human-Human Hybridoma Cells Transfected By pSV.2-Neo Gene,” Cytotechnology 6 (3): 219-226; Tolstoshev (1993) “Gene Therapy, Concepts, Current Trials And Future Directions,” Ann. Rev. Pharmacol. Toxicol. 32:573-596; Mulligan (1993) “The Basic Science of Gene Therapy,” Science 260:926-932; and Morgan et al. (1993) “Human gene therapy,” Ann. Rev. Biochem. 62:191-217). Methods commonly known in the art of recombinant DNA technology which can be used are described in Ausubel et al. (eds.), 1993, CURRENT PROTOCOLS IN MOLECULAR BIOLOGY, John Wiley & Sons, NY; Kriegler, 1990, GENE TRANSFER AND EXPRESSION, A LABORATORY MANUAL, Stockton Press, NY; and in Chapters 12 and 13, Dracopoli et al. (eds), 1994, CURRENT PROTOCOLS IN HUMAN GENETICS, John Wiley & Sons, NY.; Colbere-Garapin et al. (1981) “A New Dominant Hybrid Selective Marker For Higher Eukaryotic Cells,” J. Mol. Biol. 150:1-14; and hygro, which confers resistance to hygromycin (Santerre et al. (1984) “Expression Of Prokaryotic Genes For Hygromycin B And G418 Resistance As Dominant-Selection Markers In Mouse L Cells,” Gene 30:147-156).

The expression levels of a recombinant AFFIMER® agent protein can be increased by vector amplification (for a review, see Bebbington and Hentschel, “The Use of Vectors Based On Gene Amplification For The Expression Of Cloned Genes In Mammaian Cells,” in DNA CLONING, Vol. 3. (Academic Press, New York, 1987)). When a marker in the vector system expressing a recombinant AFFIMER® agent protein is amplifiable, increase in the level of inhibitor present in culture of host cell will increase the number of copies of the marker gene. Since the amplified region is associated with the nucleotide sequence of the recombinant AFFIMER® agent protein, production of the recombinant AFFIMER® agent protein will also increase (Crouse et al. (1983) “Expression and Amplification of Engineered Mouse Dihydrofolate Reductase Minigenes,” Mol. Cell. Biol. 3:257-266).

Where the AFFIMER® agent is an AFFIMER® polypeptide-antibody fusion or other multiprotein complex, the host cell may be co-transfected with two expression vectors, for instance the first vector encoding a heavy chain and the second vector encoding a light chain derived polypeptide, one or both of which includes an AFFIMER® polypeptide coding sequence. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot (1986) “Expression and Amplification Of Engineered Mouse Dihydrofolate Reductase Minigenes,” Nature 322:562-565; Kohler (1980) “Immunoglobulin Chain Loss In Hybridoma Lines,” Proc. Natl. Acad. Sci. (U.S.A.) 77:2197-2199). The coding sequences for the heavy and light chains may comprise cDNA or genomic DNA.

In general, glycoproteins produced in a particular cell line or transgenic animal will have a glycosylation pattern that is characteristic for glycoproteins produced in the cell line or transgenic animal. Therefore, the particular glycosylation pattern of the recombinant AFFIMER® agent protein will depend on the particular cell line or transgenic animal used to produce the protein. In some embodiments of AFFIMER® polypeptide-antibody fusions, a glycosylation pattern comprising only non-fucosylated N-glycans may be advantageous, because in the case of antibodies this has been shown to typically exhibit more potent efficacy than fucosylated counterparts both in vitro and in vivo (See for example, Shinkawa et al., J. Biol. Chem. 278:3466-3473 (2003); U.S. Pat. Nos. 6,946,292 and 7,214,775).

Further, expression of an AFFIMER® agent from production cell lines can be enhanced using a number of known techniques. For example, the glutamine synthetase gene expression system (the GS system) is a common approach for enhancing expression under certain conditions. The GS system is discussed in whole or part in connection with European Patent NOS: 0216846, 0256055, and 0323997 and European Patent Application No. 89303964.4. Thus, in some embodiments of the disclosure, the mammalian host cells (e.g., CHO) lack a glutamine synthetase gene and are grown in the absence of glutamine in the medium wherein, however, the polynucleotide encoding the immunoglobulin chain comprises a glutamine synthetase gene which complements the lack of the gene in the host cell. Such host cells containing the binder or polynucleotide or vector as discussed herein as well as expression methods, as discussed herein, for making the binder using such a host cell are part of the present disclosure.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

The recombinant AFFIMER® agent proteins produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Isolated proteins can also be physically characterized using such techniques as proteolysis, mass spectrometry (MS), nuclear magnetic resonance (NMR), high performance liquid chromatography (HPLC), and x-ray crystallography.

In some embodiments, recombinant AFFIMER® agent proteins produced in bacterial culture can be isolated, for example, by initial extraction from cell pellets, followed by at least one concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

### VI. Methods of Use and Pharmaceutical Compositions

The AFFIMER® agents of the disclosure are useful in a variety of applications including, but not limited to, therapeutic treatment methods, such as immunotherapy for cancer. In some embodiments, AFFIMER® agents described herein are useful for activating, promoting, increasing, and/or enhancing an immune response, inhibiting tumor growth, reducing tumor volume, inducing tumor regression, increasing tumor cell apoptosis, and/or reducing the tumorigenicity of a tumor. The polypeptides or agents of the disclosure, in some embodiments, are useful for immunotherapy against pathogens, such as viruses. For example, the AFFIMER® agents described herein may be useful for inhibiting viral infection, reducing viral infection, increasing virally-infected cell apoptosis, and/or increasing killing of virus-infected cells. The methods of use may be in vitro, ex vivo, or in vivo methods.

In the cancer disease state, the interaction of PD-L1 on the tumor cells with PD-1 on a T-cell reduces T-cell function signals to prevent the immune system from attacking the tumor cells. Use of an inhibitor that blocks the interaction of PD-L1 with the PD-1 receptor can prevent the cancer from evading the immune system in this way. Several PD-1 and PD-L1 inhibitors are being tested within the clinic for use in advanced melanoma, non-small cell lung cancer, renal cell carcinoma, bladder cancer and Hodgkin lymphoma, amongst other cancer types.

Immunotherapy with these immune checkpoint inhibitors appears to shrink tumors in a higher number of patients across a wider range of tumor types and is associated with lower toxicity levels than other immunotherapies, with durable responses. However, de novo and acquired resistance is still seen in a large proportion of patients. Thus, PD-L1 inhibitors, such as the PD-L1 AFFIMER® agents as provided herein, are considered to be the most promising drug category for many different cancers.

The present disclosure provides methods for activating an immune response in a subject using an AFFIMER® agent. In some embodiments, the disclosure provides methods for promoting an immune response in a subject using an AFFIMER® agent described herein. In some embodiments, the disclosure provides methods for increasing an immune response in a subject using an AFFIMER® agent. In some embodiments, the disclosure provides methods for enhancing an immune response in a subject using an AFFIMER® agent. In some embodiments, the activating, promoting, increasing, and/or enhancing of an immune response comprises increasing cell-mediated immunity. In some embodiments, the activating, promoting, increasing, and/or enhancing of an immune response comprises increasing Th1-type responses. In some embodiments, the activating, promoting, increasing, and/or enhancing of an immune response comprises increasing T-cell activity. In some embodiments, the activating, promoting, increasing, and/or enhancing of an immune response comprises increasing CD4+ T-cell activity. In some embodiments, the activating, promoting, increasing, and/or enhancing of an immune response comprises increasing CD8+ T-cell activity. In some embodiments, the activating, promoting, increasing, and/or enhancing of an immune response comprises increasing CTL activity. In some embodiments, the activating, promoting, increasing, and/or enhancing of an immune response comprises increasing NK cell activity. In some embodiments, the activating, promoting, increasing, and/or enhancing of an immune response comprises increasing T-cell activity and increasing NK cell activity. In some embodiments, the activating, promoting, increasing, and/or enhancing of an immune response comprises increasing CU activity and increasing NK cell activity. In some embodiments, the activating, promoting, increasing, and/or enhancing of an immune response comprises inhibiting or decreasing the suppressive activity of Treg cells. In some embodiments, the activating, promoting, increasing, and/or enhancing of an immune response comprises inhibiting or decreasing the suppressive activity of MDSCs. In some embodiments, the activating, promoting, increasing, and/or enhancing of an immune response comprises increasing the number of the percentage of memory T-cells. In some embodiments, the activating, promoting, increasing, and/or enhancing of an immune response comprises increasing long-term immune memory function. In some embodiments, the activating, promoting, increasing, and/or enhancing of an immune response comprises increasing long-term memory. In some embodiments, the activating, promoting, increasing, and/or enhancing of an immune response comprises no evidence of substantial side effects and/or immune-based toxicities. In some embodiments, the activating, promoting, increasing, and/or enhancing of an immune response comprises no evidence of cytokine release syndrome (CRS) or a cytokine storm. In some embodiments, the immune response is a result of antigenic stimulation. In some embodiments, the antigenic stimulation is a tumor cell. In some embodiments, the antigenic stimulation is cancer. In some embodiments, the antigenic stimulation is a pathogen. In some embodiments, the antigenic stimulation is a virally-infected cell.

In vivo and in vitro assays for determining whether an AFFIMER® agent activates, or inhibits an immune response are known in the art.

In some embodiments, a method of increasing an immune response in a subject comprises administering to the subject a therapeutically effective amount of an AFFIMER® agent described herein, wherein an AFFIMER® agent binds human PD-L1. In some embodiments, a method of increasing an immune response in a subject comprises administering to the subject a therapeutically effective amount of an AFFIMER® agent described herein, wherein the AFFIMER® agent is an AFFIMER®-containing antibody or receptor trap fusion polypeptide including an AFFIMER® polypeptide that specifically binds to PD-L1. In some embodiments, a method of increasing an immune response in a subject comprises administering to the subject a therapeutically effective amount of an encoded AFFIMER® construct, wherein the encoded AFFIMER® construct, when expressed in the patient, produces a recombinant AFFIMER® agent including an PD-L1 AFFIMER® polypeptide.

In some embodiments of the methods described herein, a method of activating or enhancing a persistent or long-term immune response to a tumor comprises administering to a subject a therapeutically effective amount of an AFFIMER® agent which binds human PD-L1. In some embodiments, a method of activating or enhancing a persistent immune response to a tumor comprises administering to a subject a therapeutically effective amount of an AFFIMER® agent described herein, wherein the AFFIMER® agent is an AFFIMER®-containing antibody or receptor trap fusion polypeptide including an AFFIMER® polypeptide that specifically binds to PD-L1. In some embodiments, a method of activating or enhancing a persistent immune response to a tumor comprises administering to a subject a therapeutically effective amount of an encoded AFFIMER® construct, wherein the encoded AFFIMER® construct, when expressed in the patient, produces a recombinant AFFIMER® agent including an PD-L1 AFFIMER® polypeptide.

In some embodiments of the methods described herein, a method of inducing a persistent or long-term immunity which inhibits tumor relapse or tumor regrowth comprises administering to a subject a therapeutically effective amount of an AFFIMER® agent which binds human PD-L1. In some embodiments, a method of inducing a persistent immunity which inhibits tumor relapse or tumor regrowth comprises administering to a subject a therapeutically effective amount of an AFFIMER® agent described herein, wherein the AFFIMER® agent is an AFFIMER® polypeptide-containing antibody or receptor trap fusion polypeptide including an AFFIMER® polypeptide that specifically binds to PD-L1. In some embodiments, a method of inducing a persistent immunity which inhibits tumor relapse or tumor regrowth comprises administering to a subject a therapeutically effective amount of an encoded AFFIMER® construct, wherein the encoded AFFIMER® construct, when expressed in the patient, produces a recombinant AFFIMER® agent including an PD-L1 AFFIMER® polypeptide.

In some embodiments of the methods described herein, a method of inhibiting tumor relapse or tumor regrowth comprises administering to a subject a therapeutically effective amount of an AFFIMER® agent which binds human PD-L1. In some embodiments, a method of inhibiting tumor relapse or tumor regrowth comprises administering to a subject a therapeutically effective amount of an AFFIMER® agent described herein, wherein the AFFIMER® agent is an AFFIMER®-containing antibody or receptor trap fusion polypeptide including an AFFIMER® polypeptide that specifically binds to PD-L1. In some embodiments, a method of inhibiting tumor relapse or tumor regrowth comprises administering to a subject a therapeutically effective amount of an encoded AFFIMER® construct, wherein the encoded AFFIMER® construct, when expressed in the patient, produces a recombinant AFFIMER® agent including PD-L1 AFFIMER® polypeptide.

In some embodiments, the tumor expresses or overexpresses a tumor antigen that is targeted by an additional binding entity provided in the AFFIMER® agent along with the PD-L1 AFFIMER® polypeptide, e.g., where the AFFIMER® agent is a bispecific or multispecific agent.

In some embodiments, the method of inhibiting growth of a tumor comprises administering to a subject a therapeutically effective amount of an AFFIMER® agent described herein. In some embodiments, the subject is a human. In some embodiments, the subject has a tumor, or the subject had a tumor which was removed.

In some embodiments, the tumor is a solid tumor. In some embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In some embodiments, the tumor is a colorectal tumor. In some embodiments, the tumor is an ovarian tumor. In some embodiments, the tumor is a lung tumor. In some embodiments, the tumor is a pancreatic tumor. In some embodiments, the tumor is a melanoma tumor. In some embodiments, the tumor is a bladder tumor.

To further illustrate, the subject AFFIMER® agents can be used to treat patients suffering from cancer, such as osteosarcoma, rhabdomyosarcoma, neuroblastoma, kidney cancer, leukemia, renal transitional cell cancer, bladder cancer, Wilm's cancer, ovarian cancer, pancreatic cancer, breast cancer (including triple negative breast cancer), prostate cancer, bone cancer, lung cancer (e.g., small cell or non-small cell lung cancer), gastric cancer, colorectal cancer, cervical cancer, synovial sarcoma, head and neck cancer, squamous cell carcinoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, carcinoid cancer or liver cancer, breast cancer or gastric cancer. In some embodiments of the disclosure, the cancer is metastatic cancer, e.g., of the varieties described above.

In some embodiments, the cancer is a hematologic cancer. In some embodiment, the cancer is selected from the group consisting of: acute myelogenous leukemia (AML), Hodgkin lymphoma, multiple myeloma, T-cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T-cell lymphoma (CTCL).

The present disclosure also provides pharmaceutical compositions comprising an AFFIMER® agent described herein and a pharmaceutically acceptable vehicle. In some embodiments, the pharmaceutical compositions find use in immunotherapy. In some embodiments, the pharmaceutical compositions find use in immuno-oncology. In some embodiments, the compositions find use in inhibiting tumor growth. In some embodiments, the pharmaceutical compositions find use in inhibiting tumor growth in a subject (e.g., a human patient). In some embodiments, the compositions find use in treating cancer. In some embodiments, the pharmaceutical compositions find use in treating cancer in a subject (e.g., a human patient).

Formulations are prepared for storage and use by combining a purified AFFIMER® agent of the present disclosure with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

In some embodiments, an AFFIMER® agent described herein is lyophilized and/or stored in a lyophilized form. In some embodiments, a formulation comprising an AFFIMER® agent described herein is lyophilized.

Suitable pharmaceutically acceptable vehicles include but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22.sup.nd Edition, 2012, Pharmaceutical Press, London).

The pharmaceutical compositions of the present disclosure can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present disclosure, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The AFFIMER® agents described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22.sup.nd Edition, 2012, Pharmaceutical Press, London.

In some embodiments, pharmaceutical formulations include an AFFIMER® agent of the present disclosure complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In some embodiments, sustained-release preparations comprising AFFIMER® agents described herein can be produced. Suitable examples of sustained-release preparations include semi-permeable matrices of solid hydrophobic polymers containing an AFFIMER® agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™. (injectable microspheres composed of lactic acid-glycolic acid copolymer and leuprolide acetate), sucrose acetate isobutyrate, and poly-D-(−)-3-hydroxybutyric acid.

In some embodiments, in addition to administering an AFFIMER® agent described herein, the method or treatment further comprises administering at least one additional immune response stimulating agent. In some embodiments, the additional immune response stimulating agent includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, IL-12, IL-15, IL-18), a checkpoint inhibitor, an antibody that blocks immunosuppressive functions (e.g., an anti-CTLA-4 antibody, anti-CD28 antibody, anti-CD3 antibody), a toll-like receptor (e.g., TLR4, TLR7, TLR9), or a member of the B7 family (e.g., CD80, CD86). An additional immune response stimulating agent can be administered prior to, concurrently with, and/or subsequently to, administration of the AFFIMER® agent. Pharmaceutical compositions comprising an AFFIMER® agent and the immune response stimulating agent(s) are also provided. In some embodiments, the immune response stimulating agent comprises 1, 2, 3, or more immune response stimulating agents.

In some embodiments, in addition to administering an AFFIMER® agent described herein, the method or treatment further comprises administering at least one additional therapeutic agent. An additional therapeutic agent can be administered prior to, concurrently with, and/or subsequently to, administration of the AFFIMER® agent. Pharmaceutical compositions comprising an AFFIMER® agent and the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the AFFIMER® agent. Combination therapy may decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some embodiments of the methods described herein, the combination of an AFFIMER® agent described herein and at least one additional therapeutic agent results in additive or synergistic results. In some embodiments, the combination therapy results in an increase in the therapeutic index of the AFFIMER® agent. In some embodiments, the combination therapy results in an increase in the therapeutic index of the additional therapeutic agent(s). In some embodiments, the combination therapy results in a decrease in the toxicity and/or side effects of the AFFIMER® agent. In some embodiments, the combination therapy results in a decrease in the toxicity and/or side effects of the additional therapeutic agent(s).

Useful classes of therapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, anti-metabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In some embodiments, the second therapeutic agent is an alkylating agent, an antimetabolite, an antimitotic, a topoisomerase inhibitor, or an angiogenesis inhibitor.

Therapeutic agents that may be administered in combination with the AFFIMER® agent described herein include chemotherapeutic agents. Thus, in some embodiments, the method or treatment involves the administration of an AFFIMER® agent of the present disclosure in combination with a chemotherapeutic agent or in combination with a cocktail of chemotherapeutic agents. Treatment with an AFFIMER® agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4.sup.th Edition, 2008, M. C. Perry, Editor, Lippincott, Williams & Wilkins, Philadelphia, Pa.

Chemotherapeutic agents useful in the present disclosure include but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In some embodiments, the additional therapeutic agent is cisplatin. In some embodiments, the additional therapeutic agent is carboplatin.

In some embodiments of the methods described herein, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In some embodiments, the chemotherapeutic agent is an anti-metabolite. An anti-metabolite is a chemical with a structure that is similar to a metabolite required for normal biochemical reactions, yet different enough to interfere with at least one normal function of cells, such as cell division. Anti-metabolites include but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In some embodiments, the additional therapeutic agent is gemcitabine.

In some embodiments of the methods described herein, the chemotherapeutic agent is an antimitotic agent, including, but not limited to, agents that bind tubulin. In some embodiments, the agent is a taxane. In some embodiments, the agent is paclitaxel or docetaxel, or a pharmaceutically acceptable salt, acid, or derivative of paclitaxel or docetaxel. In some embodiments, the agent is paclitaxel (TAXOL), docetaxel (TAXOTERE), albumin-bound paclitaxel (nab-paclitaxel; ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In some embodiments, the additional therapeutic agent is paclitaxel. In some embodiments, the additional therapeutic agent is nab-paclitaxel.

In some embodiments of the methods described herein, an additional therapeutic agent comprises an agent such as a small molecule. For example, treatment can involve the combined administration of an AFFIMER® agent of the present disclosure with a small molecule that acts as an inhibitor against tumor-associated antigens including, but not limited to, EGFR, HER2 (ErbB2), and/or VEGF. In some embodiments, an AFFIMER® agent of the present disclosure is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In some embodiments of the methods described herein, the additional therapeutic agent is a small molecule that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an inhibitor of the Hippo pathway. In some embodiments, the additional therapeutic agent is an inhibitor of the mTOR/AKR pathway. In some embodiments, the additional therapeutic agent is an inhibitor of the RSPO/LGR pathway.

In some embodiments of the methods described herein, an additional therapeutic agent comprises a biological molecule, such as an antibody. For example, treatment can involve the combined administration of an AFFIMER® agent of the present disclosure with antibodies against tumor-associated antigens including, but not limited to, antibodies that bind EGFR, HER2/ErbB2, and/or VEGF. In some embodiments, the additional therapeutic agent is an antibody specific for a cancer stem cell marker. In some embodiments, the additional therapeutic agent is an antibody that binds a component of the Notch pathway. In some embodiments, the additional therapeutic agent is an antibody that binds a component of the Wnt pathway. In some embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits beta.-catenin signaling. In some embodiments, the additional therapeutic agent is an antibody that is an angiogenesis inhibitor (e.g., an anti-VEGF or VEGF receptor antibody). In some embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

In some embodiments of the methods described herein, the additional therapeutic agent is an antibody that modulates the immune response. In some embodiments, the additional therapeutic agent is an anti-PD-1 antibody, an anti-LAG-3 antibody, an anti-CTLA-4 antibody, an anti-TIM-3 antibody, or an anti-TIGIT antibody.

Furthermore, treatment with an AFFIMER® agent described herein can include combination treatment with other biologic molecules, such as at least one cytokine (e.g., lymphokines, interleukins, tumor necrosis factors, and/or growth factors) or can be accompanied by surgical removal of tumors, removal of cancer cells, or any other therapy deemed necessary by a treating physician. In some embodiments, the additional therapeutic agent is an immune response stimulating agent.

In some embodiments of the methods described herein, the AFFIMER® agent can be combined with a growth factor selected from the group consisting of: adrenomedullin (AM), angiopoietin (Ang), BMPs, BDNF, EGF, erythropoietin (EPO), FGF, GDNF, G-CSF, GM-CSF, GDF9, HGF, HDGF, IGF, migration-stimulating factor, myostatin (GDF-8), NGF, neurotrophins, PDGF, thrombopoietin, TGF-α, TGF-β, TNF-α, VEGF, PIGF, IL-1, IL-2, IL-3, IL-4, IL-5, IL-6, IL-7, IL-12, IL-15, and IL-18.

In some embodiments of the methods described herein, the additional therapeutic agent is an immune response stimulating agent. In some embodiments, the immune response stimulating agent is selected from the group consisting of granulocyte-macrophage colony stimulating factor (GM-CSF), macrophage colony stimulating factor (M-CSF), granulocyte colony stimulating factor (G-CSF), interleukin 3 (IL-3), interleukin 12 (IL-12), interleukin 1 (IL-1), interleukin 2 (IL-2), B7-1 (CD80), B7-2 (CD86), 4-1BB ligand, anti-CD3 antibody, anti-CTLA-4 antibody, anti-TIGIT antibody, anti-PD-1 antibody, anti-LAG-3 antibody, and anti-TIM-3 antibody.

In some embodiments of the methods described herein, an immune response stimulating agent is selected from the group consisting of: a modulator of PD-1 activity, a modulator of PD-L² activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, and an immunostimulatory oligonucleotide.

In some embodiments of the methods described herein, an immune response stimulating agent is selected from the group consisting of: a PD-1 antagonist, a PD-L2 antagonist, a CTLA-4 antagonist, a CD80 antagonist, a CD86 antagonist, a KIR antagonist, a Tim-3 antagonist, a LAG3 antagonist, a TIGIT antagonist, a CD20 antagonist, a CD96 antagonist, and/or an IDO1 antagonist.

In some embodiments of the methods described herein, the PD-1 antagonist is an antibody that specifically binds PD-1. In some embodiments, the antibody that binds PD-1 is KEYTRUDA (MK-3475), pidilizumab (CT-011), nivolumab (OPDIVO, BMS-936558, MDX-1106), MEDI0680 (AMP-514), REGN2810, BGB-A317, PDR-001, or STI-A1110. In some embodiments, the antibody that binds PD-1 is described in PCT Publication WO 2014/179664, for example, an antibody identified as APE2058, APE1922, APE1923, APE1924, APE 1950, or APE1963, or an antibody containing the CDR regions of any of these antibodies. In other embodiments, the PD-1 antagonist is a fusion protein that includes PD-L2, for example, AMP-224. In other embodiments, the PD-1 antagonist is a peptide inhibitor, for example, AUNP-12.

In some embodiments, the CTLA-4 antagonist is an antibody that specifically binds CTLA-4. In some embodiments, the antibody that binds CTLA-4 is ipilimumab (YERVOY) or tremelimumab (CP-675,206). In some embodiments, the CTLA-4 antagonist a CTLA-4 fusion protein, for example, KAHR-102.

In some embodiments, the LAG3 antagonist is an antibody that specifically binds LAG3. In some embodiments, the antibody that binds LAG3 is IMP701, IMP731, BMS-986016, LAG525, and GSK2831781. In some embodiments, the LAG3 antagonist includes a soluble LAG3 receptor, for example, IMP321.

In some embodiments, the KIR antagonist is an antibody that specifically binds KIR. In some embodiments, the antibody that binds KIR is lirilumab.

In some embodiments, an immune response stimulating agent is selected from the group consisting of: a CD28 agonist, a 4-1BB agonist, an OX40 agonist, a CD27 agonist, a CD80 agonist, a CD86 agonist, a CD40 agonist, and a GITR agonist. p In some embodiments, the OX40 agonist includes OX40 ligand, or an OX40-binding portion thereof. For example, the OX40 agonist may be MEDI6383. In some embodiments, the OX40 agonist is an antibody that specifically binds OX40. In some embodiments, the antibody that binds OX40 is MEDI6469, MEDI0562, or MOXR0916 (RG7888). In some embodiments, the OX40 agonist is a vector (e.g., an expression vector or virus, such as an adenovirus) capable of expressing OX40 ligand. In some embodiments the OX40-expressing vector is Delta-24-RGDOX or DNX2401.

In some embodiments, the 4-1BB (CD137) agonist is a binding molecule, such as an anticalin. In some embodiments, the anticalin is PRS-343. In some embodiments, the 4-1BB agonist is an antibody that specifically binds 4-1BB. In some embodiments, antibody that binds 4-1BB is PF-2566 (PF-05082566) or urelumab (BMS-663513).

In some embodiments, the CD27 agonist is an antibody that specifically binds CD27. In some embodiments, the antibody that binds CD27 is varlilumab (CDX-1127).

In some embodiments, the GITR agonist comprises GITR ligand or a GITR-binding portion thereof. In some embodiments, the GITR agonist is an antibody that specifically binds GITR. In some embodiments, the antibody that binds GITR is TRX518, MK-4166, or INBRX-110.

In some embodiments, immune response stimulating agents include but are not limited to, cytokines such as chemokines, interferons, interleukins, lymphokines, and members of the tumor necrosis factor (TNF) family. In some embodiments, immune response stimulating agents include immunostimulatory oligonucleotides, such as CpG dinucleotides.

In some embodiments, an immune response stimulating agent includes, but is not limited to, anti-PD-1 antibodies, anti-PD-L2 antibodies, anti-CTLA-4 antibodies, anti-CD28 antibodies, anti-CD80 antibodies, anti-CD86 antibodies, anti-4-1BB antibodies, anti-OX40 antibodies, anti-KIR antibodies, anti-Tim-3 antibodies, anti-LAG3 antibodies, anti-CD27 antibodies, anti-CD40 antibodies, anti-GITR antibodies, anti-TIGIT antibodies, anti-CD20 antibodies, anti-CD96 antibodies, or anti-IDO1 antibodies.

In some embodiments, the AFFIMER® agents disclosed herein may be used alone, or in association with radiation therapy.

In some embodiments, the AFFIMER® agents disclosed herein may be used alone, or in association with targeted therapies. Examples of targeted therapies include: hormone therapies, signal transduction inhibitors (e.g., EGFR inhibitors, such as cetuximab (Erbitux) and erlotinib (Tarceva)); HER2 inhibitors (e.g., trastuzumab (Herceptin) and pertuzumab (Perjeta)); BCR-ABL inhibitors (such as imatinib (Gleevec) and dasatinib (Sprycel)); ALK inhibitors (such as crizotinib (Xalkori) and ceritinib (Zykadia)); BRAF inhibitors (such as vemurafenib (Zelboraf) and dabrafenib (Tafinlar)), gene expression modulators, apoptosis inducers (e.g., bortezomib (Velcade) and carfilzomib (Kyprolis)), angiogenesis inhibitors (e.g., bevacizumab (Avastin) and ramucirumab (Cyramza), monoclonal antibodies attached to toxins (e.g., brentuximab vedotin (Adcetris) and ado-trastuzumab emtansine (Kadcyla)).

In some embodiments, the AFFIMER® agents of the disclosure may be used in combination with an anti-cancer therapeutic agent or immunomodulatory drug such as an immunomodulatory receptor inhibitor, e.g., an antibody or antigen-binding fragment thereof that specifically binds to the receptor.

In some embodiments of the disclosure, an AFFIMER® agent is administered in with a STING agonist, for example, as part of a pharmaceutical composition. The cyclic-di-nucleotides (CDNs) cyclic-di-AMP (produced by Listeria monocytogenes and other bacteria) and its analogs cyclic-di-GMP and cyclic-GMP-AMP are recognized by the host cell as a pathogen associated molecular pattern (PAMP), which bind to the pathogen recognition receptor (PRR) known as Stimulator of INterferon Genes (STING). STING is an adaptor protein in the cytoplasm of host mammalian cells which activates the TANK binding kinase (TBK1)-IRF3 and the NF-kappa.B signaling axis, resulting in the induction of IFN-beta. and other gene products that strongly activate innate immunity. It is now recognized that STING is a component of the host cytosolic surveillance pathway, that senses infection with intracellular pathogens and in response induces the production of IFN-α, leading to the development of an adaptive protective pathogen-specific immune response consisting of both antigen-specific CD4+ and CD8+ T cells as well as pathogen-specific antibodies. U.S. Pat. Nos. 7,709,458 and 7,592,326; PCT Publication NOS: WO2007/054279, WO2014/093936, WO2014/179335, WO2014/189805, WO2015/185565, WO2016/096174, WO2016/145102, WO2017/027645, WO2017/027646, and WO2017/075477; and Yan et al., Bioorg. Med. Chem Lett. 18:5631-4, 2008.

In some embodiments of the disclosure, an AFFIMER® agent of the disclosure is administered in association with an Akt inhibitor. Exemplary AKT inhibitors include GDC0068 (also known as GDC-0068, ipatasertib and RG7440), MK-2206, perifosine (also known as KRX-0401), GSK690693, AT7867, triciribine, CCT128930, A-674563, PHT-427, Akti-1/2, afuresertib (also known as GSK2110183), AT13148, GSK2141795, BAY1125976, uprosertib (aka GSK2141795), Akt Inhibitor VIII (1,3-dihydro-1-[1-[4-(6-phenyl-1H-imidazo[4,5-g]quinoxalin-7-yl)phenyl]m-ethyl]-4-piperidinyl]-2H-benzimidazol-2-one), Akt Inhibitor X (2-chloro-N,N-diethyl-10H-phenoxazine-10-butanamine, monohydrochloride), MK-2206 (8-(4-(1-aminocyclobutyl)phenyl)-9-phenyl-[1,2,4]triazolo[3,4-f] [-1,6]naphthyridin-3 (2H)-one), uprosertib (N—((S)-1-amino-3-(3,4-difluorophenyl) propan-2-yl)-5-chloro-4-(4-chloro-1-methyl-1H-pyrazol-5-yl) furan-2-carboxamide), ipatasertib ((S)-2-(4-chlorophenyl)-1-(4-((5R,7R)-7-hydroxy-5-methyl-6,7-dihydro-5H-c-yclopenta[d]pyrimidin-4-yl) piperazin-1-yl)-3-(isopropylamino) propan-1-one)-, AZD 5363 (4-Piperidinecarboxamide, 4-amino-N-[(1S)-1-(4-chlorophenyl)-3-hydroxypropyl]-1-(7H-pyrrolo[2,3-d]p-yrimidin-4-yl)), perifosine, GSK690693, GDC-0068, tricirbine, CCT128930, A-674563, PF-04691502, AT7867, miltefosine, PHT-427, honokiol, triciribine phosphate, and KP372-1A (10H-indeno[2,1-e]tetrazolo[1,5-b][1,2,4]triazin-10-one), Akt Inhibitor IX (CAS 98510-80-6). Additional Akt inhibitors include: ATP-competitive inhibitors, e.g. isoquinoline-5-sulfonamides (e.g., H-8, H-89, NL-71-101), azepane derivatives (e.g., (−)-balanol derivatives), aminofurazans (e.g., GSK690693), heterocyclic rings (e.g., 7-azaindole, 6-phenylpurine derivatives, pyrrolo[2,3-d]pyrimidine derivatives, CCT128930, 3-aminopyrrolidine, anilinotriazole derivatives, spiroindoline derivatives, AZD5363, A-674563, A-443654), phenylpyrazole derivatives (e.g., AT7867, AT13148), thiophenecarboxamide derivatives (e.g., Afuresertib (GSK2110183), 2-pyrimidyl-5-amidothiophene derivative (DC120), uprosertib (GSK2141795); Allosteric inhibitors, e.g., 2,3-diphenylquinoxaline analogues (e.g., 2,3-diphenylquinoxaline derivatives, triazolo[3,4-f][1,6]naphthyridin-3 (2H)-one derivative (MK-2206)), alkylphospholipids (e.g., Edelfosine (1-O-octadecyl-2-O-methyl-rac-glycero-3-phosphocholine, ET-18-OCH3) ilmofosine (BM 41.440), miltefosine (hexadecylphosphocholine, HePC), perifosine (D-21266), erucylphosphocholine (ErPC), erufosine (ErPC3, erucylphosphohomocholine), indole-3-carbinol analogues (e.g., indole-3-carbinol, 3-chloroacetylindole, diindolylmethane, diethyl 6-methoxy-5,7-dihydroindolo[2,3-b]carbazole-2,10-dicarboxylate (SR13668), OSU-A9), Sulfonamide derivatives (e.g., PH-316, PHT-427), thiourea derivatives (e.g., PIT-1, PIT-2, DM-PIT-1, N-[(1-methyl-1H-pyrazol-4-yl) carbonyl]-N′-(3-bromophenyl)-thiourea), purine derivatives (e.g., Triciribine (TCN, NSC 154020), triciribine mono-phosphate active analogue (TCN-P), 4-amino-pyrido[2,3-d]pyrimidine derivative API-1, 3-phenyl-3H-imidazo[4,5-b]pyridine derivatives, ARQ 092), BAY 1125976, 3-methyl-xanthine, quinoline-4-carboxamide, 2-[4-(cyclohexa-1,3-dien-1-yl)-1H-pyrazol-3-yl]phenol, 3-oxo-tirucallic acid, 3.alpha.-and 3.beta.-acetoxy-tirucallic acids, acetoxy-tirucallic acid; and irreversible inhibitors, e.g., natural products, antibiotics, Lactoquinomycin, Frenolicin B, kalafungin, medermycin, Boc-Phe-vinyl ketone, 4-hydroxynonenal (4-HNE), 1,6-naphthyridinone derivatives, and imidazo-1,2-pyridine derivatives.

In some embodiments of the disclosure, an AFFIMER® agent of the disclosure is administered in association with a MEK inhibitor. Exemplary MEK inhibitors include AZD6244 (Selumetinib), PD0325901, GSK1120212 (Trametinib), U0126-EtOH, PD184352, RDEA119 (Rafametinib), PD98059, BIX 02189, MEK162 (Binimetinib), AS-703026 (Pimasertib), SL-327, BIX02188, AZD8330, TAK-733, cobimetinib and PD318088.

In some embodiments of the disclosure, an AFFIMER® agent of the disclosure is administered in association with both an anthracycline such as doxorubicin and cyclophosphamide, including pegylated liposomal doxorubicin.

In some embodiments of the disclosure, an AFFIMER® agent of the disclosure is administered in association with both an anti-CD20 antibody and an anti-CD3 antibody, or a bispecific CD20/CD3 binder (including a CD20/CD3 BiTE).

In some embodiments of the disclosure, an AFFIMER® agent of the disclosure is administered in association with a CD73 inhibitor, a CD39 inhibitor or both. These inhibitors can be CD73 binders or CD39 binders (such as antibody, antibody fragments or antibody mimetics) that inhibit the ectonucleosidase activity. The inhibitor may be a small molecule inhibitor of the ectonucleosidase activity, such as 6-N,N-Diethyl-β-γ-dibromomethylene-D-adenosine-5′-triphosphate trisodium salt hydrate, PSB069, PSB 06126,

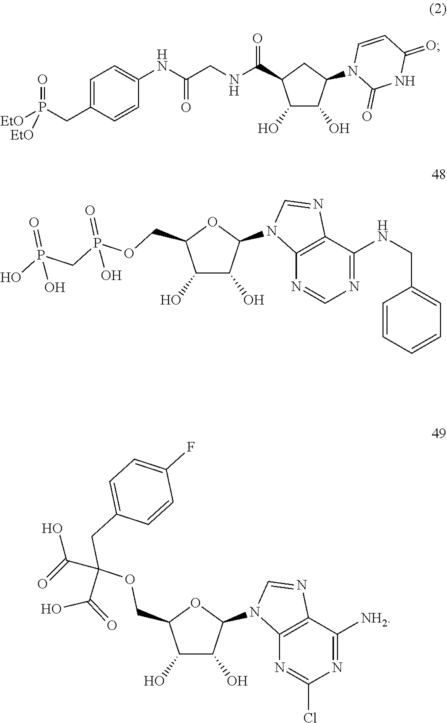 [SMILES: CCOP(=O)(Cc1ccc(NC(=O)CNC(=O)[C@H]2C[C@@H](n3ccc(=O)[nH]c3=O)[C@H](O)[C@@H]2O)cc1)OCC ; O=P(O)(O)CP(=O)(O)OC[C@H]1O[C@@H](n2cnc3c(NCc4ccccc4)ncnc32)[C@H](O)[C@@H]1O ; Nc1nc(Cl)nc2c1ncn2[C@@H]1O[C@H](COC(Cc2ccc(F)cc2)(C(=O)O)C(=O)O)[C@@H](O)[C@H]1O]

In some embodiments of the disclosure, an AFFIMER® agent of the disclosure is administered in association with an inhibitor poly ADP ribose polymerase (PARP). Exemplary PARP inhibitors include Olaparib, Niraparib, Rucaparib, Talazoparib, Veliparib, CEP9722, MK4827 and BGB-290.

In some embodiments of the disclosure, an AFFIMER® agent of the disclosure is administered in association with an oncolytic virus. An exemplary oncolytic virus is Talimogene Laherparepvec.

In some embodiments of the disclosure, an AFFIMER® agent of the disclosure is administered in association with an CSF-1 antagonist, such as an agent that binds to CSF-1 or CSF1R and inhibits the interaction of CSF-1 with CSF1R on macrophage. Exemplary CSF-1 antagonists include Emactuzumab and FPA008.

In some embodiments of the disclosure, an AFFIMER® agent of the disclosure is administered in association with an anti-CD38 antibody. Exemplary anti-CD39 antibodies include Daratumumab and Isatuximab.

In some embodiments of the disclosure, an AFFIMER® agent of the disclosure is administered in association with an anti-CD40 antibody. Exemplary anti-CD40 antibodies include Selicrelumab and Dacetuzumab.

In some embodiments of the disclosure, an AFFIMER® agent of the disclosure is administered in association with an inhibitor of anaplatic lymphoma kinase (ALK). Exemplary ALK inhibitors include Alectinib, Crizotinib and Ceritinib.

In some embodiments of the disclosure, an AFFIMER® agent of the disclosure is administered in association with multikinase inhibitor that inhibits at least one selected from the group consisting of the family members of VEGFR, PDGFR and FGFR, or an anti-angiogenesis inhibitor. Exemplary inhibitors include Axitinib, Cediranib, Linifanib, Motesanib, Nintedanib, Pazopanib, Ponatinib, Regorafenib, Sorafenib, Sunitinib, Tivozanib, Vatalanib, LY2874455, or SU5402.

In some embodiments of the disclosure, an AFFIMER® agent of the disclosure is administered in conjunction with at least one vaccine intended to stimulate an immune response to at least one predetermined antigen. The antigen(s) may be administered directly to the individual, or may be expressed within the individual from, for example, a tumor cell vaccine (e.g., GVAX) which may be autologous or allogenic, a dendritic cell vaccine, a DNA vaccine, an RNA vaccine, a viral-based vaccine, a bacterial or yeast vaccine (e.g., a Listeria monocytogenes or Saccharomyces cerevisiae), etc. See, e.g., Guo et al., Adv. Cancer Res. 2013; 119:421-475; Obeid et al., Semin Oncol. 2015 August; 42 (4): 549-561. The target antigen may also be a fragment or fusion polypeptide comprising an immunologically active portion of the antigens listed in the table.

In some embodiments of the disclosure, an AFFIMER® agent of the disclosure is administered in association with at least one antiemetic including, but not limited to: casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl by Pfizer; New York, N.Y.), hydroxyzine (sold as Atarax by Pfizer; New York, N.Y.), metoclopramide (sold as Reglan by AH Robins Co.; Richmond, Va.), lorazepam (sold as Ativan by Wyeth; Madison, N.J.), alprazolam (sold as Xanax by Pfizer; New York, N.Y.), haloperidol (sold as Haldol by Ortho-McNeil; Raritan, N.J.), droperidol (Inapsine), dronabinol (sold as Marinol by Solvay Pharmaceuticals, Inc.; Marietta, Ga.), dexamethasone (sold as Decadron by Merck and Co.; Rahway, N.J.), methylprednisolone (sold as Medrol by Pfizer; New York, N.Y.), prochlorperazine (sold as Compazine by Glaxosmithkline; Research Triangle Park, N.C.), granisetron (sold as Kytril by Hoffmann-La Roche Inc.; Nutley, N.J.), ondansetron (sold as Zofran by Glaxosmithkline; Research Triangle Park, N.C.), dolasetron (sold as Anzemet by Sanofi-Aventis; New York, N.Y.), tropisetron (sold as Navoban by Novartis; East Hanover, N.J.).

Other side effects of cancer treatment include red and white blood cell deficiency. Accordingly, in some embodiments of the disclosure, an AFFIMER® agent is administered in association with an agent which treats or prevents such a deficiency, such as, e.g., filgrastim, PEG-filgrastim, erythropoietin, epoetin alfa or darbepoetin alfa.

In some embodiments of the disclosure, an AFFIMER® agent of the disclosure is administered in association with anti-cancer radiation therapy. For example, in some embodiments of the disclosure, the radiation therapy is external beam therapy (EBT): a method for delivering a beam of high-energy X-rays to the location of the tumor. The beam is generated outside the patient (e.g., by a linear accelerator) and is targeted at the tumor site. These X-rays can destroy the cancer cells and careful treatment planning allows the surrounding normal tissues to be spared. No radioactive sources are placed inside the patient's body. In some embodiments of the disclosure, the radiation therapy is proton beam therapy: a type of conformal therapy that bombards the diseased tissue with protons instead of X-rays. In some embodiments of the disclosure, the radiation therapy is conformal external beam radiation therapy: a procedure that uses advanced technology to tailor the radiation therapy to an individual's body structures. In some embodiments of the disclosure, the radiation therapy is brachytherapy: the temporary placement of radioactive materials within the body, usually employed to give an extra dose—or boost—of radiation to an area.

In some embodiments of the methods described herein, the treatment involves the administration of an AFFIMER® agent of the present disclosure in combination with anti-viral therapy. Treatment with an AFFIMER® agent can occur prior to, concurrently with, or subsequent to administration of antiviral therapy. The anti-viral drug used in combination therapy will depend upon the virus the subject is infected with.

Combined administration can include co-administration, either in a single pharmaceutical formulation or using separate formulations, or consecutive administration in either order but generally within a time period such that all active agents can exert their biological activities simultaneously.

It will be appreciated that the combination of an AFFIMER® agent described herein and at least one additional therapeutic agent may be administered in any order or concurrently. In some embodiments, the AFFIMER® agent will be administered to patients that have previously undergone treatment with a second therapeutic agent. In certain other embodiments, the AFFIMER® agent and a second therapeutic agent will be administered substantially simultaneously or concurrently. For example, a subject may be given an AFFIMER® agent while undergoing a course of treatment with a second therapeutic agent (e.g., chemotherapy). In some embodiments, an AFFIMER® agent will be administered within 1 year of the treatment with a second therapeutic agent. In certain alternative embodiments, an AFFIMER® agent will be administered within 10, 8, 6, 4, or 2 months of any treatment with a second therapeutic agent. In certain other embodiments, an AFFIMER® agent will be administered within 4, 3, 2, or 1 weeks of any treatment with a second therapeutic agent. In some embodiments, an AFFIMER® agent will be administered within 5, 4, 3, 2, or 1 days of any treatment with a second therapeutic agent. It will further be appreciated that the two (or more) agents or treatments may be administered to the subject within a matter of hours or minutes (e.g., substantially simultaneously).

For the treatment of a disease, the appropriate dosage of an AFFIMER® agent of the present disclosure depends on the type of disease to be treated, the severity and course of the disease, the responsiveness of the disease, whether the AFFIMER® agent is administered for therapeutic or preventative purposes, previous therapy, the patient's clinical history, and so on, all at the discretion of the treating physician. The AFFIMER® agent can be administered one time or over a series of treatments lasting from several days to several months, or until a cure is affected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates. In some embodiments, dosage is from 0.01 μg to 100 mg/kg of body weight, from 0.1 μg to 100 mg/kg of body weight, from 1 μg to 100 mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In some embodiments, the dosage of the AFFIMER® agent is from about 0.1 mg to about 20 mg/kg of body weight. In some embodiments, the dosage of the AFFIMER® agent is about 0.1 mg/kg of body weight. In some embodiments, the dosage of the AFFIMER® agent is about 0.25 mg/kg of body weight. In some embodiments, the dosage of the AFFIMER® agent is about 0.5 mg/kg of body weight. In some embodiments, the dosage of the AFFIMER® agent is about 1 mg/kg of body weight. In some embodiments, the dosage of the AFFIMER® agent is about 1.5 mg/kg of body weight. In some embodiments, the dosage of the AFFIMER® agent is about 2 mg/kg of body weight. In some embodiments, the dosage of the AFFIMER® agent is about 2.5 mg/kg of body weight. In some embodiments, the dosage of the AFFIMER® agent is about 5 mg/kg of body weight. In some embodiments, the dosage of the AFFIMER® agent is about 7.5 mg/kg of body weight. In some embodiments, the dosage of the AFFIMER® agent is about 10 mg/kg of body weight. In some embodiments, the dosage of the AFFIMER® agent is about 12.5 mg/kg of body weight. In some embodiments, the dosage of the AFFIMER® agent is about 15 mg/kg of body weight. In some embodiments, the dosage can be given once or more daily, weekly, monthly, or yearly. In some embodiments, the AFFIMER® agent is given once every week, once every two weeks, once every three weeks, or once every four weeks.

In some embodiments, an AFFIMER® agent may be administered at an initial higher “loading” dose, followed by at least one lower dose. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. In some embodiments, a dosing regimen comprises administering an initial loading dose, followed by maintenance doses of, for example one-half of the initial dose every other week. In some embodiments, a dosing regimen comprises administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, the AFFIMER® agent is administered for 3, 4, 5, 6, 7, 8, or more cycles. For example, the AFFIMER® agent is administered every 2 weeks for 6 cycles, the AFFIMER® agent is administered every 3 weeks for 6 cycles, the AFFIMER® agent is administered every 2 weeks for 4 cycles, the AFFIMER® agent is administered every 3 weeks for 4 cycles, etc. Dosing schedules can be decided upon and subsequently modified by those skilled in the art.

Thus, the present disclosure provides methods of administering to a subject the polypeptides or agents described herein comprising using an intermittent dosing strategy for administering at least one agent (e.g., two or three agents), which may reduce side effects and/or toxicities associated with administration of an AFFIMER® agent, chemotherapeutic agent, etc. In some embodiments, a method for treating cancer in a human subject comprises administering to the subject a therapeutically effective dose of an AFFIMER® agent in combination with a therapeutically effective dose of a chemotherapeutic agent, wherein one or both of the agents are administered according to an intermittent dosing strategy. In some embodiments, the intermittent dosing strategy comprises administering an initial dose of an AFFIMER® agent to the subject and administering subsequent doses of the AFFIMER® agent about once every 2 weeks. In some embodiments, the intermittent dosing strategy comprises administering an initial dose of an AFFIMER® agent to the subject and administering subsequent doses of the AFFIMER® agent about once every 3 weeks. In some embodiments, the intermittent dosing strategy comprises administering an initial dose of an AFFIMER® agent to the subject and administering subsequent doses of the AFFIMER® agent about once every 4 weeks. In some embodiments, the AFFIMER® agent is administered using an intermittent dosing strategy and the chemotherapeutic agent is administered weekly.

In some embodiments, the disclosure also provides methods for treating subjects using an AFFIMER® agent of the disclosure, wherein the subject suffers from a viral infection. In some embodiments, the viral infection is infection with a virus selected from the group consisting of human immunodeficiency virus (HIV), hepatitis virus (A, B, or C), herpes virus (e.g., VZV, HSV-I, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus or arboviral encephalitis virus.

In some embodiments, the disclosure provides methods for treating subjects using an AFFIMER® agent thereof of the disclosure, wherein the subject suffers from a bacterial infection. In some embodiments, the bacterial infection is infection with a bacterium selected from the group consisting of Chlamydia, rickettsial bacteria, mycobacteria, staphylococci, streptococci, pneumonococci, meningococci and gonococci, klebsiella, proteus, serratia, pseudomonas, Legionella, Corynebacterium diphtheriae, Salmonella, bacilli, Vibrio cholerae, Clostridium tetan, Clostridium botulinum, Bacillus anthricis, Yersinia pestis, Mycobacterium leprae, Mycobacterium lepromatosis, and Borriella.

In some embodiments, the disclosure provides methods for treating subjects using an AFFIMER® agent of the disclosure, wherein the subject suffers from a fungal infection. In some embodiments, the fungal infection is infection with a fungus selected from the group consisting of Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

In some embodiments, the disclosure provides methods for treating subjects using an AFFIMER® agent of the disclosure, wherein the subject suffers from a parasitic infection. In some embodiments, the parasitic infection is infection with a parasite selected from the group consisting of Entamoeba histolytica, Balantidium coli, Naegleria fowleri, Acanthamoeba, Giardia lambia, Cryptosporidium, Pneumocystis carinii, Plasmodium vivax, Babesia microti, Trypanosoma brucei, Trypanosoma cruzi, Leishmania donovani, Toxoplasma gondii and Nippostrongylus brasiliensis.

### EXAMPLES

### Example 1: Selection of Human PD-L1 Binding AFFIMER® Polypeptides from Phage Display Library

Selections from a phage display library comprising random loop sequences nine amino acids in length displayed in a constant AFFIMER® framework type III. Such selection procedures are generally known. According to such procedures, suspensions of phage were incubated with target antigen (either biotinylated antigen captured on streptavidin beads or unbiotinylated antigen captured on a plate). Unbound phages were then washed away and, subsequently, bound phages were eluted by incubating the antigen with a low pH solution, followed by a high pH solution. E. coli were then infected with the released, pH neutralized phage solution, and a preparation of first round phages was obtained. The cycle was performed repeatedly, for example, two or three times and, in order to enrich for targeting phages. In some instances, the stringency conditions were increased in the later rounds of selection, for example by increasing the number of wash steps, reducing the antigen concentration, and/or preselecting with blocked streptavidin beads or wells coated with blocking reagent.

Following selection by successive rounds of phage amplification, PD-L1 binding clones were identified by a soluble AFFIMER® polypeptide ELISA. Briefly, AFFIMER® clones were overexpressed from a phagemid vector, the bacterial cell was lysed, and the lysate was used in the ELISA to detect AFFIMER® polypeptide binding to PD-L1-Fc (R&D Systems). For detection, the lysate was immobilized on a plate with an antibody conjugated to the His tag on the AFFIMER® polypeptide. Clones showing specific binding were sequenced to identify loop sequences. To illustrate, selection of PD-L1 binding phage from the AFFIMER® library was carried out as described below using approximately 1×10¹² phage. Biotinylated antigen was captured on M280 streptavidin or neutravidin beads (Thermo Scientific), and target PD-L1-Fc protein was biotinylated in-house using the EZ Link Sulfo-NHS-LC Biotin kit (Pierce). The sequences of the clones isolated that bind to human PD-L1-Fc are detailed in Tables 1-4.

### Example 2: Expression of Anti PD-L1 AFFIMER® Polypeptides from E. coli

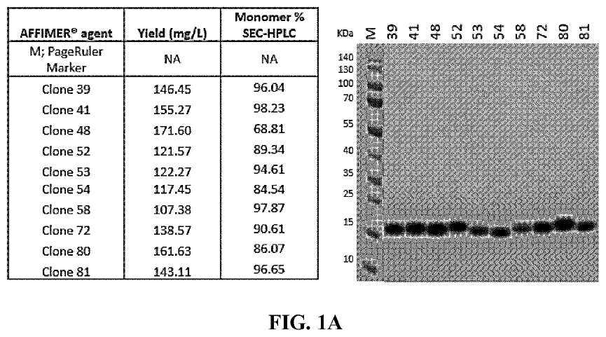

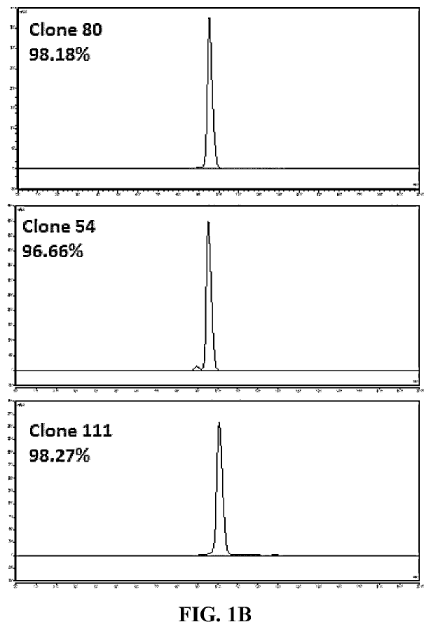

The 200 ng expression plasmid pD861 (Atum) was transformed into BL21 E. coli cells (Millipore) using the manufacturer's protocol. The total transformed cell mixture was plated onto LB agar plates containing 50 ug/ml kanamycin (AppliChem) and incubated at 37° C. overnight. The following day, the lawn of transformed E. coli was transferred to a sterile flask of 1×broth media (Melford) and 50 μg/ml Kanamycin and incubated at 30° C., shaking at 250 rpm. Expression was induced with 10 mM Rhamnose (Alfa Aesar) once the cells reached an OD600 of 0.8-1.0, following which the culture was incubated for 5 hours at 37° C. Cells were harvested by centrifuging at 4,500 rpm for 1 hour. For culture volumes less than 500 ml, the E. coli cell pellet was lysed by resuspending in 1:10 NPI20 buffer (50 mM Sodium phosphate, 0.5M NaCl, 20 mM Imidazole (Sigma)) supplemented with 0.5 ml 10×BugBuster per gram of wet cell paste (Millipore), lysozyme (Applichem) and Benzonase (Millipore). Cells were lysed for 1 hour at room temperature on a bottle roller. For culture volumes greater than 500 ml, the cell pellet was resuspended in 1:10 supplemented NP120 and sonicated for 2 minutes (10 seconds on/off cycles). Following lysis, the solution was centrifuged at 20,000×g for 1 hour at 4° C. Batch bind affinity purification of the His tagged protein from clarified supernatant was performed using Nickel agarose affinity resin (Super-NiNTA500; Generon). An appropriate volume of NiNTA resin (binding capacity 1 ml per 20 mg protein) was washed with 5 column volumes (CV) of water to remove storage solution and then equilibrated with 5 CV NPI20 buffer using gravity flow in a StEP™ column (Thompson). Resin was incubated with clarified E. coli solution for 1 hour at room temperature. Then, the solution was passed through a StEP™ column by gravity flow and the resin was washed with 5CV NPI20 buffer. Bound protein was eluted off the resin with 5 CV of NPI400 (50 mM Sodium phosphate, 0.5M NaCl, 0.4M Imidazole (Sigma)). Eluted protein was desalted into 1×PBS using Centripure columns (emp Biotech GmbH). The protein yield was estimated using a Nanodrop (Thermo) A280 reading. All purified AFFIMER® clones were found to be expressed at greater than 100 mg/L (FIG. 1A).

Preparative SEC was performed for some protein batches using HiLoad 26/600 Superdex 75 pg run in PBS 1× at 2.6 ml/min flow rate on an AKTA system (Cytiva). Eluted protein samples were run on SDS-PAGE Bolt Bis Tris plus 4-12% gel in Novex™ 20× Bolt™ MES SDS running buffer (Thermo Scientific) at 200 Volts with reducing sample buffer for 10 minutes at 95° C. Protein bands on gel were stained with Quick Coomassie (Generon). PageRuler prestained protein molecular weight marker (Thermo Scientific) was run on each gel to confirm the molecular weight (MW) and purify of proteins purified (FIG. 1A). Purified proteins were then run-on size exclusion chromatography HPLC (SEC-HPLC) in PBS 1× running buffer on an Acclaim SEC-300 column run on an Ultimate 3000 HPLC System (Thermo Scientific) at 0.7 ml/min flow rate. Type III AFFIMER® clones had a high monomer purity, >98%, as illustrated in FIG. 1B.

### Example 3: Human PD-L1 Binding Biacore Kinetics

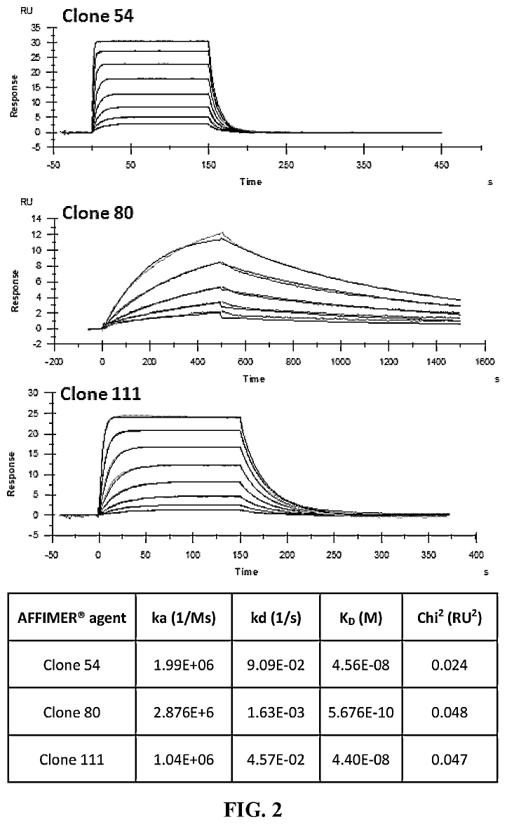

BIACORE™ T200 binding kinetic analysis was performed on monomer AFFIMER® clones with running buffer HBS-EP+ (Cytiva) and a series S sensor CM5 chip immobilized with human PD-L1-Fc (R&D Systems) in 10 mM Sodium acetate pH4.0 μsing amine coupling reagents (Cytiva). A concentration titration of AFFIMER® monomer was run as analyte with an association time of 150 seconds, followed by a dissociation time of 300 seconds at a flow rate of 30 μl/min. For Clone 80, the association time was increased to 500 seconds and 1000 seconds dissociation time. The human PD-L1-Fc immobilized surface was regenerated with 3-3.5 mM NaOH solution (Cytiva) for 20-30 seconds at 20 μl/min flow rate. The kinetic data was blank subtracted and fit to a 1:1 Langmuir binding model (BIAcore evaluation software; Cytiva) to calculate an apparent K_{D} value. Examples of SEC-purified type III AFFIMER® clones from a naïve phage selection showed PD-L1 binding K_{D} affinities of nM-pM (FIG. 2).

### Example 4: PD-L1/PD-1 Competitive ELISA

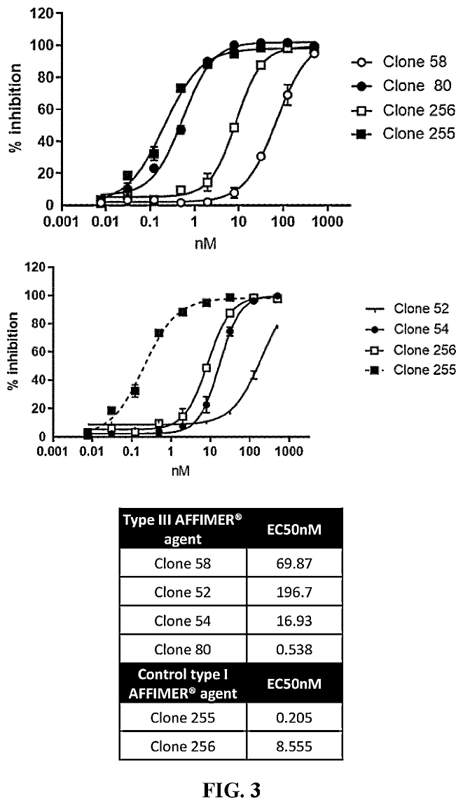

The competitive inhibition of AFFIMER® multimers was evaluated by enzyme linked immunosorbent assay (ELISA). Human PD-1-Fc (R&D Systems) was coated at 0.5 μg/ml on the plate. Plates were washed two times with 150 ml of washing buffer (PBS, Tween 20 0.1%) with a plate washer and saturated with Casein 5% (Sigma) in PBS for 90 minutes at room temperature (25±1° C.). Plates were subsequently washed. AFFIMER® polypeptides and controls (human PD-1-Fc; R&D Systems or blank) were then diluted in duplicate, and preincubated with huPD-L1-Fc (R&D Systems) at a predefined concentration equivalent to the EC₈₀ for 30 minutes then loaded on the plate for 90 minutes at room temperature (25±1° C.). Plates were washed 3 times. Biotinylated polyclonal antibody anti-human PD-L1 (R&D Systems) was then diluted in dilution buffer and incubated 90 minutes at room temperature (25±1° C.). Plates were washed 3 times and Streptavidin HRP was incubated for 30 minutes at room temperature (25±1° C.). Plates were washed and the substrate (TMB; Pierce Thermo-Scientific) added to the plate for 10 minutes. The reaction was stopped using an acidic solution and plates were read at absorbance 450-630 nm. The IC₅₀ was then calculated using the interpolated non-linear four-parameter standard curve. Data shows anti-PD-L1 AFFIMER® clones are competitive for binding to PD-L¹ with PD-1, having IC₅₀ values of 0.5-200 nM. The highest affinity AFFIMER® polypeptide (SEQ ID NO: 593) has a similar IC₅₀ value to a lead type I AFFIMER® polypeptide Clone 255 (SEQ ID NO. 1118) (FIG. 3).

(SEQ ID NO: 1118)
MIPRGLSEAKPATPEIQEIVDKVKPQLEEKTNETYGKLEAVQYKTQVLAR
EGRQDWVLSTNYYIKVRAGDNKYMHLKVFNGPWVPFPHQQLADRVLTGYQ
VDKNKDDELTGF

### Example 5: Promega PD1:PD-L1 Blockade Cell-based Assay

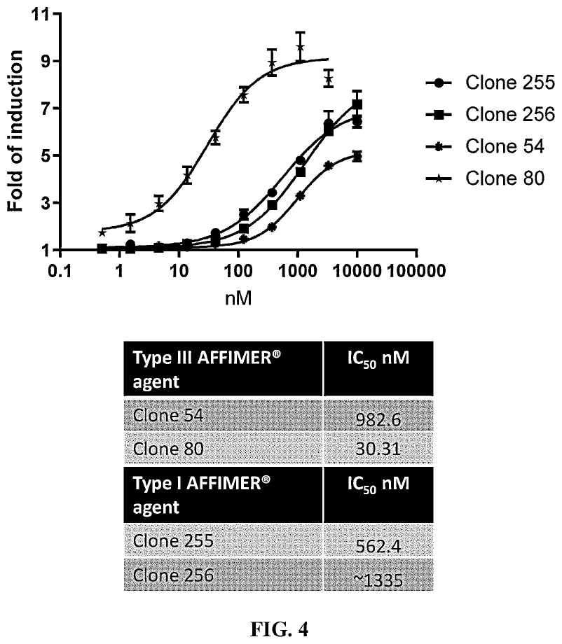

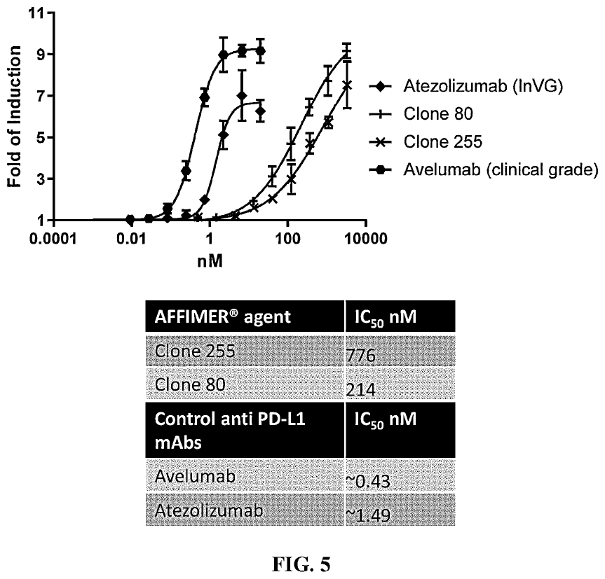

The PD-1/PD-L1 blockade Bioassay (Promega) was run according to manufacturer instructions in a 384 well plate. PD-1-expressing Jurkat T cells that also express NFAT-induced luciferase were co-cultured with human PD-L1 expressing CHO-K1 cells and a cell surface protein designed to activate cognate T-cell receptors (TCRs) in an antigen-independent manner. When there is a PD-1/PD-L1 interaction between the cells, this inhibits TCR signalling and NFAT-mediated luciferase activity. Addition of an anti-PD-L1 AFFIMER® polypeptide or antibody control releases the inhibitory signal and results in TCR signalling and NFAT-mediated luciferase activity. Two lead type III anti-PD-L1 AFFIMER® polypeptides, Clone 54 (SEQ ID NO: 567) and Clone 80 (SEQ ID NO: 593), were compared to type I AFFIMER® polypeptides, (Clone 255 SEQ ID NO: 1118 and Clone 256 SEQ ID NO: 1119), and shown to be comparable or better at blocking the PD1:PD-L1 interaction on cells (FIG. 4). Comparing Clone 80 (SEQ ID NO: 593) and type I AFFIMER® polypeptide Clone 255 (SEQ ID NO: 1118) to clinical monoclonal antibodies (mAbs) Avelumab and Atezolizumab, demonstrated that the monomeric interaction has an IC₅₀ value of 30 nM compared to 0.5-1.5 nM for mAbs which have bivalent interactions with target (FIG. 5).

(SEQ ID NO: 1119)
MIPRGLSEAKPATPEIQEIVDKVKPQLEEKTNETYGKLEAVQYKTQVLAK
EHGPDSWWSTNYYIKVRAGDNKYMHLKVFNGPQEKNQWVEEADRVLTGYQ
VDKNKDDELTGF

### Example 6: Cell Binding CHO Expressing PD-L1

Human PD-L1 CHO cells having a cell surface over expressing human PD-L1 or CHO-negative cells were plated at 50,000 cells per well in a round bottom tissue culture plate. AFFIMER® clones were titrated from 700 nM or monoclonal antibody (mAb) controls titrated from 10 nM in cell staining buffer (PBS, 1% BSA+2 mM EDTA+0.05% Sodium Azide buffer) and incubated for 1 hour. After two washes with staining buffer, AFFIMER® polypeptides were detected with an anti-cystatin antibody. After a further two washes, AF488-conjugated anti-goat antibody (to detect AFFIMER® polypeptides) or AF488-conjugated anti human IgG1 antibody (to detect mAb controls) were incubated for 40 minutes followed by an additional wash step. Viability dye (Zombie Aqua reagent, Biolegend) was added and the plates were incubated for 10 min. After a final wash, cells were fixed and the data acquired using a multiparameter flow cytometer (Guava 12HT, Luminex Corp).

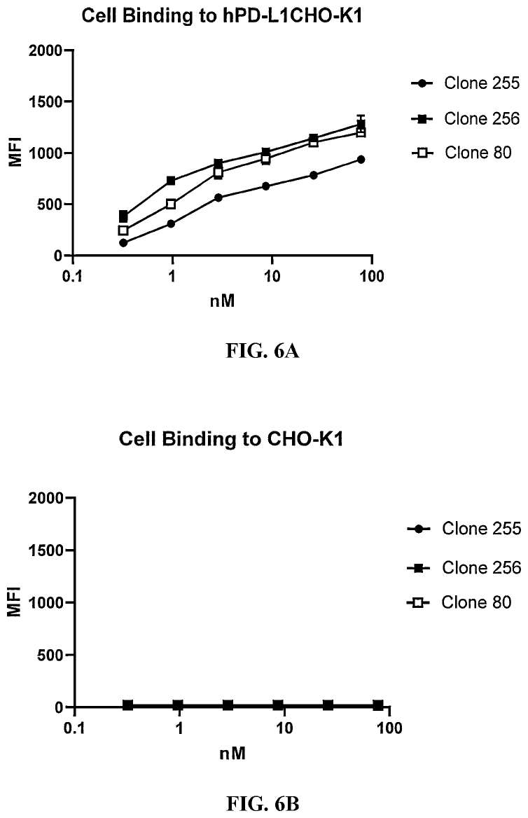

FIG. 6A shows binding signal in Median Fluorescence Intensity (MFI) of Clone 80 (SEQ ID NO: 593), Clone 255 (SEQ ID NO. 1118) and Clone 256 (SEQ ID NO. 1119) titrated from 100 nM on CHO PD-L1 expressing cells. The negative CHO cells show no non-specific binding signal (FIG. 6B). FIG. 6C shows representative cell staining histogram data on live gated cells, Clone 80 (SEQ ID NO: 593), Clone 255 (SEQ ID NO. 1118) and Clone 256 (SEQ ID NO. 1119) on hu-PD-L1 CHO cells at 100 nM.

### Example 7: Clone 80 Binding Loop Alanine Scanning

Clone 80 (SEQ ID NO: 593) was mutated throughout binding loop 2 and loop 4 positions to determine which loop, position, and amino acids are involved in the binding properties and aggregation of the molecule. A total of 18 point mutations were performed by replacing loop amino acids with an alanine residue in SEQ ID NO: 593. Specifically, overlapping forward and reverse primers were designed to carry point mutations (Integrated DNA Technologies; IDT) and site-directed mutagenesis was performed to mutate single amino acids in loop regions to alanine residues. Mutant proteins were produced from E. coli, one stage purified, and quality control was carried out on SEC-HPLC as detailed in Example 1 (Table 11).

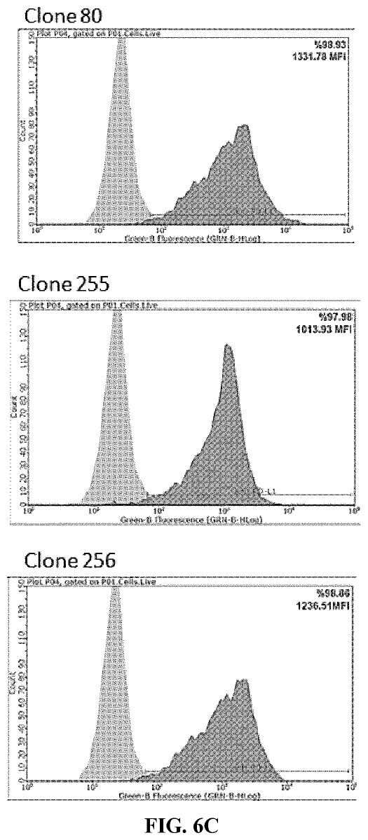

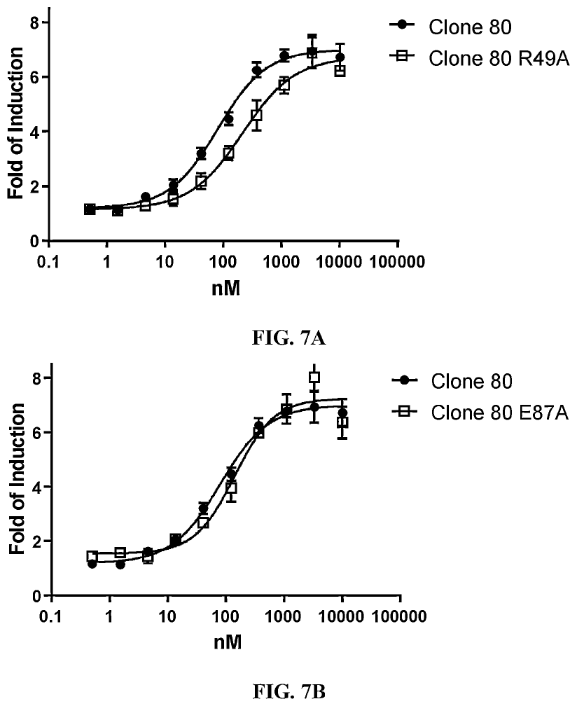

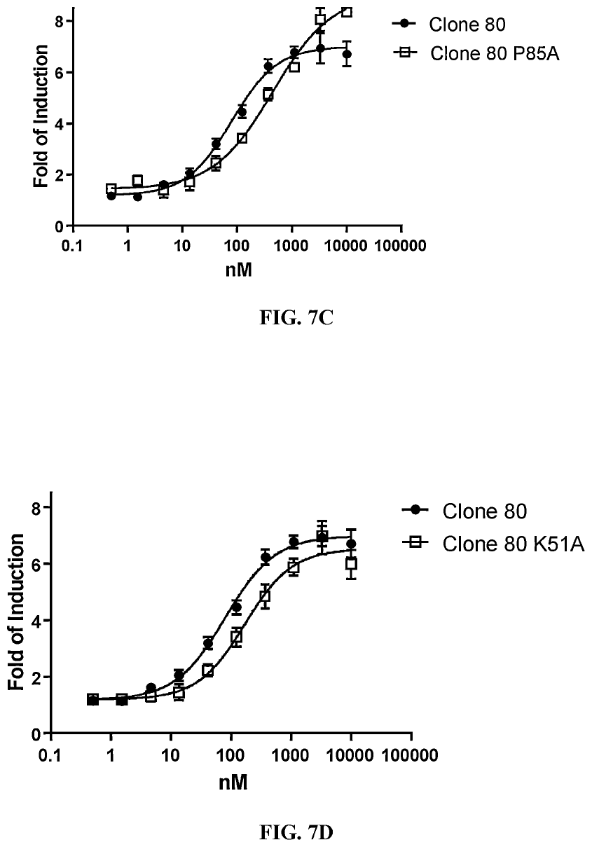

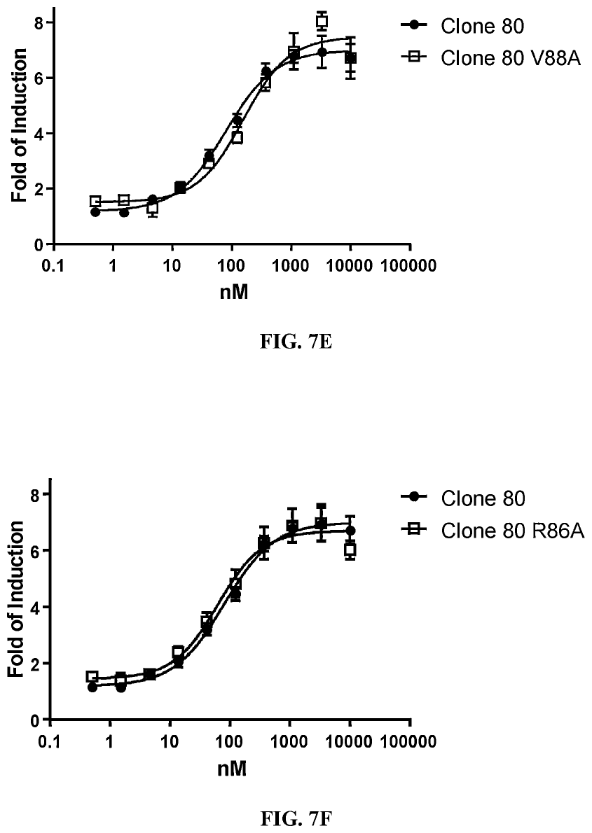

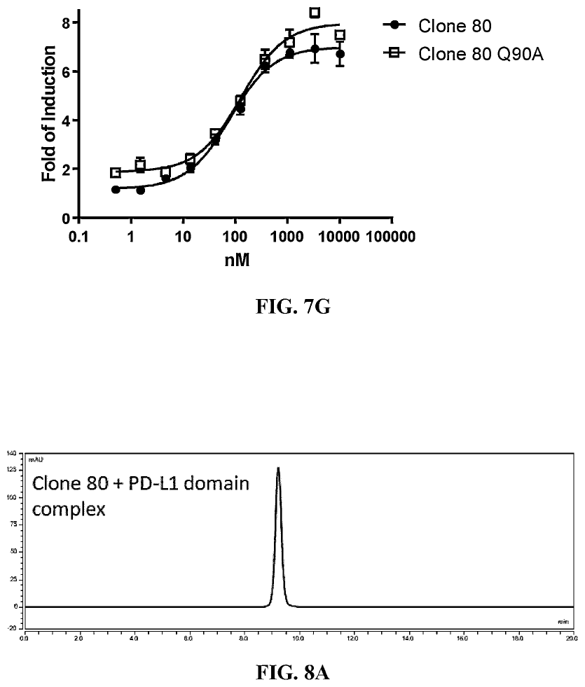

BIACORE™ kinetic analysis was carried out to assess binding to PD-L1-Fc (R&D Systems) using an 8K Biacore (Cytiva) compared to the Clone 80 parent control molecule (SEQ ID NO: 593) (FIG. 7). Analysis identified three mutants which no longer bind target antigen, three mutants which have reduced binding and twelve (12) mutants which retaining binding to target antigen. Data suggests loop 4 is less involved in direct binding to human PD-L1 target antigen. The mutant AFFIMER® clones which retained binding to PD-L1 were assessed in both a competition ELISA (as described in Example 4) and the PD1:PD-L1 blockade cell-based Promega assay (as described in Example 5). The importance of certain residues in binding was found to be as follows:

(SEQ ID NO: 85)
Loop 2: RRKHFPQWP
(SEQ ID NO: 339)
Loop 4: DLQPREVFQ,

where italicized residues are moderately involved in binding, bold residues are heavily involved in binding, and underlined residues are involved in self-association/dimerization.

TABLE 11
Clone 80 Alanine Screening

| AFFIMER® | | | | | | Bia- |
|---|---|---|---|---|---|---|
| Poly- | | SEQ | | SEQ | SEC- | core |
| peptide | Loop 2 | ID | Loop 4 | ID | HPLC % | KD |
| Mutation | mutation | NO: | mutation | NO: | purity | (nM) |
| Clone 80 | RRKHFPQWP | 85 | DLQPREVFQ | 339 | | 0.74 - |
| | | | | | | 2.3 |
| R49A | ARKHFPQWP | 1100 | — | — | 69.6 | 2.00 |
| R50A | RAKHFPQWP | 1101 | — | — | 68.25 | — |
| K51A | RRAHFPQWP | 1102 | — | — | 69.29 | 0.66 |
| H52A | RRKAFPQWP | 1103 | — | — | 75.15 | 38 |
| F53A | RRKHAPQWP | 1104 | — | — | 86.19 | — |
| P54A | RRKHFAQWP | 1105 | — | — | 73.7 | 0.93 |
| Q55A | RRKHFPAWP | 1106 | — | — | 69.51 | 1.69 |
| W56A | RRKHFPQAP | 1107 | — | — | 75.87 | — |
| P57A | RRKHFPQWA | 1108 | — | — | 85.15 | 8.91 |
| D82A | — | — | ALQPREVFQ | 1109 | 60.94 | 5.08 |
| L83A | — | — | DAQPREVFQ | 1110 | 72.76 | 1.48 |
| Q84A | — | — | DLAPREVFQ | 1111 | 77.77 | 0.61 |
| P85A | — | — | DLQAREVFQ | 1112 | 85.02 | 2.53 |
| R86A | — | — | DLQPAEVFQ | 1113 | 71.04 | 4.02 |
| E87A | — | — | DLQPRAVFQ | 1114 | 85.77 | 0.61 |
| V88A | — | — | DLQPREAFQ | 1115 | 75.5 | 1.96 |
| F89A | — | — | DLQPREVAQ | 1116 | 88.89 | 2.61 |
| Q90A | — | — | DLQPREVFA | 1117 | 74.69 | 0.97 |

Results confirmed mutating these particular residues in the AFFIMER® polypeptide loop regions of Clone 80 does not alter the blocking activity for PD-L1:PD1 interactions (Table 12 and FIG. 7).

TABLE 12
Clone 80 Alanine Mutants: PD-1/PD-L1 Competitive ELISA

| AFFIMER ® | IC₅₀ (nM) |
|---|---|
| Clone | average n = 2 |
| Clone 80 | 2.605 |
| Clone 80 R49A | 4.825 |
| Clone 80 K51A | 3.345 |
| Clone 80 R86A | 2.33 |
| Clone 80 E87A | 3.825 |
| Clone 80 V88A | 2.565 |
| Clone 80 Q90A | 2.225 |

### Example 8: PD-L1 Domain/Clone 80 AFFIMER® Protein Complex Crystallography

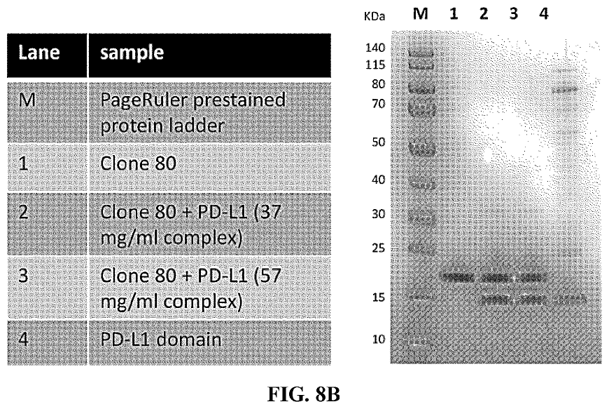

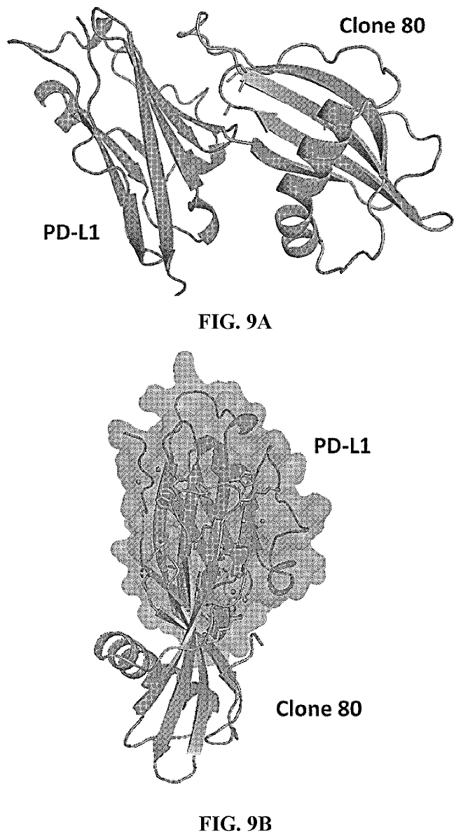

The hPD-L1 binding domain (N-terminal IgV domain 18-134) was expressed as inclusion bodies in E. coli. The cell pellet was washed with 50 mM Tris pH 8, 150 mM NaCl, 10 mM DTT. 10 mM EDTA buffer followed by a wash with an addition of 0.5% Triton 100 to the buffer. The inclusion body pellet was then solubilized in 50 mM Tris pH 8, 200 mM NaCl, 1 mM DTT, 6M guanidine HCl buffer for 16 h at 4° C. The following day the solution was centrifuged at 5000×g and 0.45 μm filtered. The protein was refolded by drop wise addition of 100 mM Tris pH 8, 1 M arginine, 0.25 mM glutathione (reduced and oxidized), 2 mM EDTA and protease inhibitor tablets (EDTA free; Roche), and the solution was stirred over night at 4° C. The following day, the protein was 10,000 ×g centrifuged for 1 h, 0.45 μm filtered and concentrated two-fold by tangential flow filtration (TFF) buffer exchanging into 10 mM Tris pH8, 20 mM NaCl. Clone 80 (SEQ ID NO: 593) was produced and purified as described in Example 2, and preparative SEC-purified protein was mixed with refolded PD-L1 antigen in a 1:1.2 AFFIMER® polypeptide-to-antigen molar ratio to form a binding complex, which was then further concentrated by TFF and passed through a preparative SEC column Hiload 26/600 Superdex 75 pg using an AKTA system (Cytiva) run at 2.6 ml/min in 10 mM Tris pH 8, 20 mM NaCl buffer. Pure complex eluted fractions were isolated, concentrated to 37 and 57 mg/ml and purity was analyzed with SEC-HPLC and SDS-PAGE analysis (FIGS. 8A-8B). To perform the crystallography, several commercial screens were set up at both +4 and +18° C. with sitting drop vapor diffusion method using ratios of 0.15:0.15 μl, 0.2:0.1 μl and 0.1:0.2 μl protein and reservoir solutions. Crystals were obtained from a number of solutions tested, many containing PEG, and a range of buffers were taken forward from the +18° C. condition. Crystals were flash-cooled in liquid nitrogen in mother liquor and the diffraction data was collected. The Clone 80/hPD-L1 domain protein complex structure was solved using the CCP4 suite using molecular replacement with models derived from previously solved AFFIMER® polypeptide structures and the PDB structure of PD-L1 Ig1 domain (excised from 4Z18) Diffraction was collected to 1.96 Å. The main interaction surface was discovered to be both polar and hydrophobic, with loop 2 of Clone 80 interacting with the human PD-L1 antigen and amino acids in frameworks both before and after loop 4 involved in binding target (FIGS. 9A-9B, Table 13) with loop 4 residues being disordered. The overall interaction area for the complex was 690 Å2. The human PD-L1 binding surface was very similar to literature examples of proteins binding PD-L1 (1. Lee, J. et al. (2016) Nature Communications 7, 13354, 2.Zak, K. et al. (2016) Oncotarget, 7, 30323, 3.Zhang F. et al (2017) Cell Discovery, 3, 17004).

TABLE 13
Amino acid interactions and classification
between Clone 80 and PD-L1

| Clone 80 | | Interaction |
|---|---|---|
| amino acid | PD-L1 amino acid | Classification |
| M1 | E58, D61, K62, N63 | Hydrophobic |
| R50 | Y56 | Hydrophobic |
| R50 | E58 | Salt bridge |
| R50 | Y56, M115 | Hydrophobic |
| H52 | Y56 | Hydrogen bond |
| H52 | I54, Q66, V68 | Hydrophobic |
| F53 | I54, Y56, M115, | Hydrophobic |
| | I116, S117, A121 | |
| W56 | M115, A121, D122, | Hydrophobic |
| | Y123 | |
| K76 | D61 | water-mediated |
| | | hydrogen bond |
| D78 | E60, D61 | Hydrophobic |
| S80 | R113 | Hydrophobic, |
| | | positive energy |
| L81 | R113, M115, Y123, | Hydrophobic |
| | R125 | |
| D92 | R125 | Salt bridge |
| V94 | E60 | Hydrophobic |
| T96 | E60, D61 | Hydrophobic |

### Example 9: AFFIMER® Polypeptide-Fc Fusion Formatting

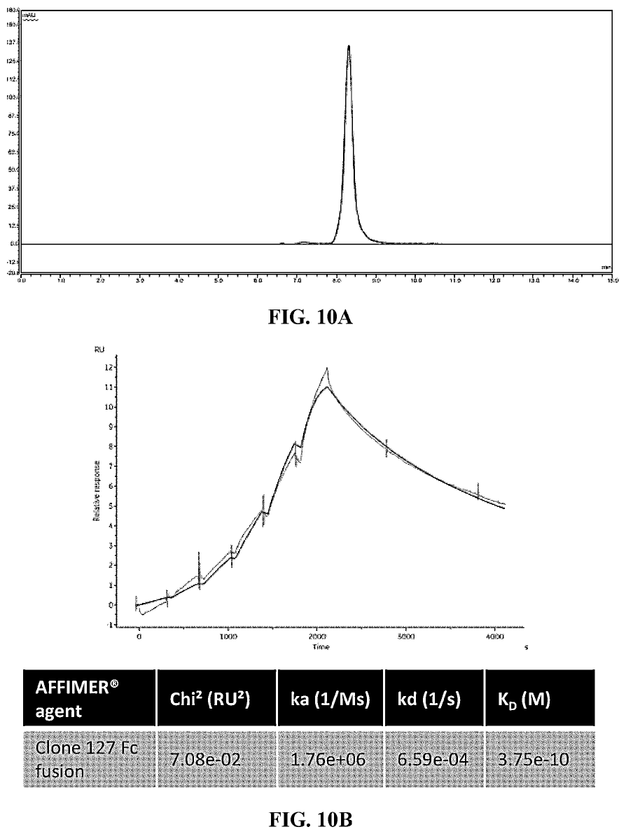

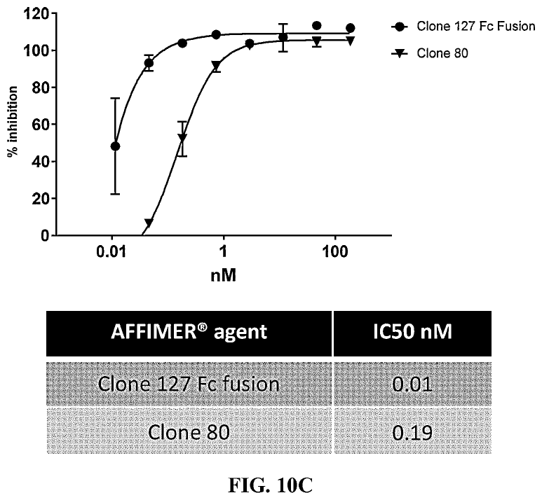

Anti-PD-L1 Clone 127 (SEQ ID NO: 640) was genetically fused with human IgG1 Fc fragment with a repetitive (G4S)₆ (SEQ ID NO: 1120) flexible linker, and the formatted AFFIMER® agent was expressed from a CMV mammalian expression vector pD609 (Atum) with a human CD33 leader sequence (Clone 127 Fc fusion, SEQ ID NO: 1121). Suspension HEK cell (Expi293F cell line; Thermo) transfections were performed with purified expression vector pD609 (Atum) using Expifectamine reagent (Thermo) following the manufacturer's protocol. Supernatant was harvested 7 days post transfection by centrifuging at 10,000 ×g for 1 hour and filtered using a 0.45 μm filter followed by a 0.22 μm filter. Protein was purified from the HEK cell supernatant using MabSelect™ PrismA resin (Cytiva). The resin was washed with five column volumes (CV) water, equilibrated with 5 CV PBS 1×, and the supernatant was run through, followed by a wash with 10 CV PBS 1×. Bound protein was eluted in 5 CV 0.1M glycine pH 3, with buffer exchanging into PBS 1× using PD MultiTrap G25 plates (Cytiva). Analytical SEC was carried out using a Yarra-3000 SEC-HPLC column (Phenomenex) run on an Ultimate 3000 HPLC (Thermo) at 0.8 ml/min in PBS 1× (FIG. 10A). BIACORE™ kinetic analysis was performed using single cycle kinetics which confirms sub nM affinity to human PD-L1-Fc antigen (FIG. 10B). A PD-1/PD-L1 competition ELISA was performed and showed the Fc fusion format to be more potent than a Clone 80 monomer (SEQ ID NO: 593) with an IC₅₀ of 0.01 nM (FIG. 10C).

### Example 10: Mammalian AFFIMER® Polypeptide In-line Fusion (ILF) Formatting and Characterization

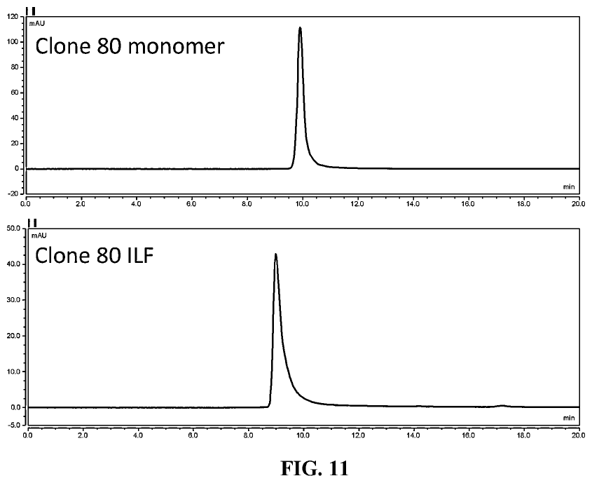

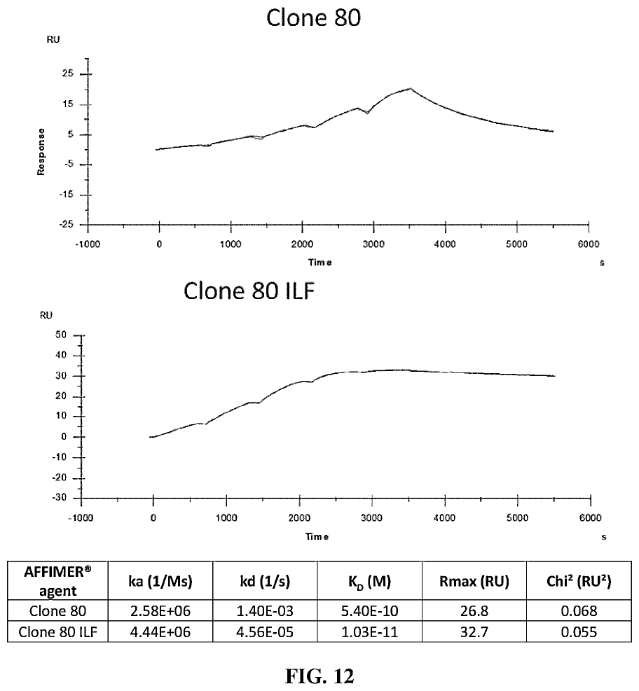

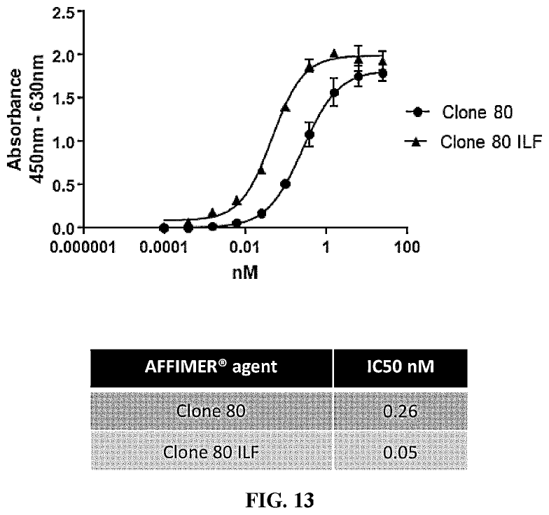

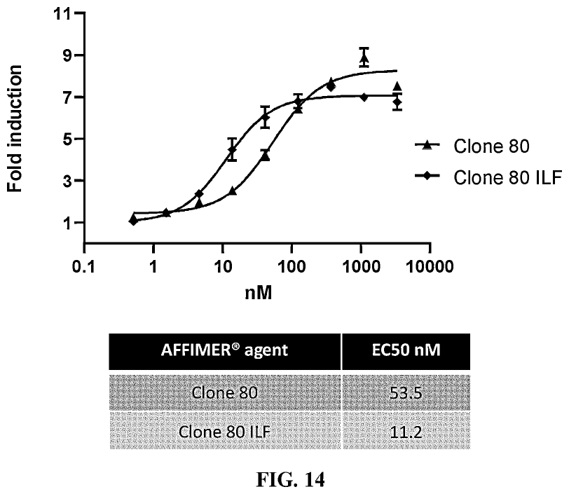

Clone 80 monomer (SEQ ID NO: 593) and an in-line fusion (ILF) homodimer fused with a repetitive rigid alpha helical linker A (EAAAK)₆ (SEQ ID NO: 1169) (Clone 80 ILF, SEQ ID NO: 1122) were expressed from HEK cells transiently as detailed in Example 9. Secreted AFFIMER® clones were purified from supernatant using Ni Sepharose Excel resin (Cytiva) as described in Example 2 and run-on SEC-HPLC to assess their purity and molecular weight (FIG. 11). BIACORE™ was run using a sensor chip Protein A (Cytiva) capturing huPD-L1-Fc (R&D System), and AFFIMER® clones were run in solution as analyte increasing from 0.125 to 2 nM titration and a dissociation time of 2000 seconds. Regeneration of the antigen captured on the chip was performed using a 10 mM glycine pH1.5 solution. Dimer ILF format data showed avidity achieved with a K_{D} of 10.3 pM compared to 0.54 nM with the AFFIMER® monomer (FIG. 12). Avidity was further shown in both the PD-L1 binding ELISA and the Promega cell-based assay, with the ILF dimer blocking the PD-L1:PD1 interaction more strongly than a mammalian produced monomer (FIGS. 13-14).

### Example 11: Staphylococcal Enterotoxin B (SEB) Assay

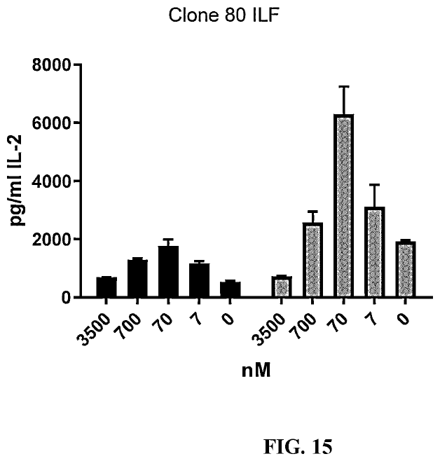

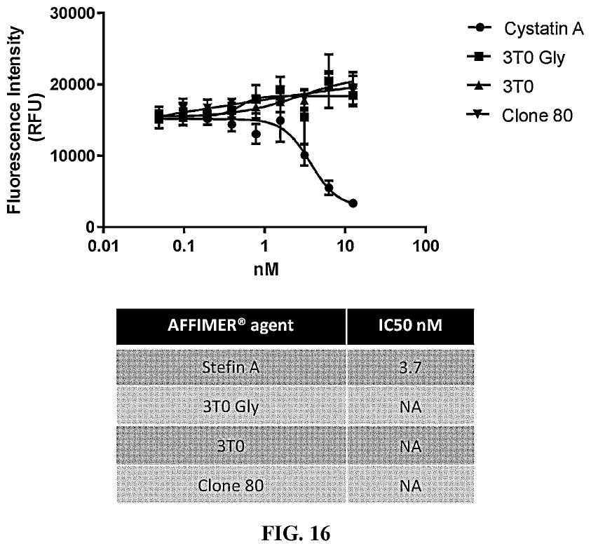

Peripheral blood monocyte cells (PBMCs) from healthy human donors (n=2) were plated at 60,000 cells per well in a 96-well round bottom tissue culture plate. AFFIMER® homodimer ILF protein or control antibody were diluted and tested with the following concentration range: 3500, 700, 70 and 7 nM. A fixed concentration (200 ng/ml) of staphylococcal enterotoxin B (SEB; Toxin Technology) was added to all wells and plates were incubated for 96 h. After incubation, plates were centrifuged, and supernatant was taken to measure the interleukin-2 (IL-2) levels by HTRF (Cisbio). IL-2 concentrations from test sample wells were compared with the basal IL-2 concentration (SEB alone control). Clone 80 ILF (SEQ ID NO: 1122) homodimer increased IL-2 production in a dose-dependent manner with the two human donors tested, and a hook effect was observed at high concentrations. The maximum effect with the highest IL-2 production was observed at 70 nM AFFIMER® dimer concentration (FIG. 15).

### Example 12: Clone 80 Monomer Cysteine Protease Inhibition Assay

Cysteine protease activity of Clone 80 (SEQ ID NO: 593) monomer and controls was measured by their ability to inhibit papain cleavage of a fluorogenic peptide substrate, N-Carbobenzyloxy-Phe-Arg-7-amido-4-methylcoumarin (Z-Phe-Arg-AMC; R&D System). Briefly, papain was activated for 15 min on ice with activation buffer containing DTT. Activated papain was pre-incubated with AFFIMER® type III control scaffold (3t0 gly or 3t0) or Cystatin A control for 15 minutes at room temperature. Following addition of 200 μM peptide substrate, fluorescence at 460 nm (excitation 360 nm) was acquired on the Pherastar (BMG Labtech) for 20 minutes in kinetic mode. The data (RFU) was linear at the 6 minute timepoint and the average of triplicate wells against log [Cystatin A or AFFIMER® polypeptide] was used to calculate IC₅₀ values using a four parameter non-linear regression curve fit. FIG. 16 shows that Stefin A has an IC₅₀ of 3.8 nM whereas Clone 80 (SEQ ID NO: 593) or the type III scaffold with or without glycine loop regions shows no inhibitory activity in this assay.

### Example 13: Clone 80 Nano DSC Tm Analysis

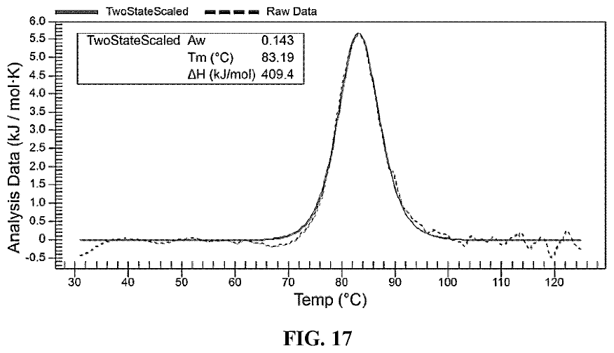

The melting temperature of Clone 80 (SEQ ID NO: 593) was measured using A nano differential scanning calorimeter (nDSC; TA Instruments) in PBS 1× buffer with a 1° C./min ramp from 30-120° C. PBS blank-subtracted data was fit to a two-state scaled model. The data in FIG. 17 shows a high thermal stability with a melting temperature (Tm) of 83.19° C. for a type III AFFIMER® polypeptide containing binding loops.

TABLE 14
Clones used in Examples - Sequences

| | | SEQ |
|---|---|---|
| | | ID |
| Clone | Sequence | NO: |
| 255 | MIPRGLSEAKPATPEIQEIVDKVKPQLEEKTNETYGKLEAVQYKTQVLAREGRQDWVLSTN | 1118 |
| | YYIKVRAGDNKYMHLKVFNGPWVPFPHQQLADRVLTGYQVDKNKDDELTGF | |
| 255 | ATGATCCCGCGTGGCCTGTCTGAAGCTAAACCAGCAACTCCGGAAATTCAAGAGATCGTCG | 1123 |
| | ATAAGGTGAAACCGCAGCTGGAAGAGAAAACGAACGAAACCTACGGTAAGCTGGAAGCGGT | |
| | CCAGTACAAAACCCAAGTGCTAGCACGTGAAGGTCGTCAGGATTGGGTTCTGTCCACCAAC | |
| | TATTACATTAAGGTTCGTGCCGGTGACAATAAGTATATGCACCTGAAAGTGTTCAACGGCC | |
| | CGTGGGTTCCATTTCCACATCAGCAGCTGGCGGACCGTGTTCTGACCGGTTACCAGGTTGA | |
| | CAAGAACAAAGATGACGAGCTGACGGGTTTC | |
| 256 | MIPRGLSEAKPATPEIQEIVDKVKPQLEEKTNETYGKLEAVQYKTQVLAKEHGPDSWWSTN | 1119 |
| | YYIKVRAGDNKYMHLKVFNGPQEKNQWVEEADRVLTGYQVDKNKDDELTGF | |
| 256 | ATGATCCCGCGTGGCCTGTCTGAAGCTAAACCAGCAACTCCGGAAATTCAAGAGATCGTCG | 1124 |
| | ATAAGGTGAAACCGCAGCTGGAAGAGAAAACGAACGAAACCTACGGTAAGCTGGAAGCGGT | |
| | CCAGTACAAAACCCAAGTGCTAGCAAAAGAACATGGTCCAGATTCTTGGTGGTCCACCAAC | |
| | TATTACATTAAGGTTCGTGCCGGTGACAATAAGTATATGCACCTGAAAGTGTTCAACGGCC | |
| | CGCAGGAAAAAAACCAGTGGGTTGAAGAAGCGGACCGTGTTCTGACCGGTTACCAGGTTGA | |
| | CAAGAACAAAGATGACGAGCTGACGGGTTTC | |
| 80 R49A | MIPGGLSEAKPATPEIQEIVDKVKPQLEEKTGETYGKLEAVQYKTQVVARKHFPQWPGTNY | 1125 |
| | YIKVRAGDNKYMHLKVFKSLDLQPREVFQEDLVLTGYQVDKNKDDELTGF | |
| 80 R49A | ATGATCCCAGGTGGCCTGAGCGAAGCAAAACCAGCAACCCCAGAGATTCAAGAAATTGTTG | 1126 |
| | ACAAAGTGAAGCCGCAGCTGGAAGAGAAAACCGGCGAAACCTACGGCAAACTGGAAGCCGT | |
| | CCAGTATAAGACTCAAGTCGTCGCTCGTAAACATTTCCCACAGTGGCCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGAAGTTTTCCAGGAAGATTTGGTGCTGACGGGCTACCAGGTTGACAA | |
| | GAACAAAGATGACGAGCTGACGGGTTTC | |
| 80 R50A | MIPGGLSEAKPATPEIQEIVDKVKPQLEEKTGETYGKLEAVQYKTQVVRAKHFPQWPGTNY | 1127 |
| | YIKVRAGDNKYMHLKVFKSLDLQPREVFQEDLVLTGYQVDKNKDDELTGF | |
| 80 R50A | ATGATCCCAGGTGGCCTGAGCGAAGCAAAACCAGCAACCCCAGAGATTCAAGAAATTGTTG | 1128 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTGCTAAACATTTCCCACAGTGGCCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGAAGTTTTCCAGGAAGATTTGGTGCTGACGGGCTACCAGGTTGACAA | |
| | GAACAAAGATGACGAGCTGACGGGTTTC | |
| 80 K51A | MIPGGLSEAKPATPEIQEIVDKVKPQLEEKTGETYGKLEAVQYKTQVVRRAHFPQWPGTNY | 1129 |
| | YIKVRAGDNKYMHLKVFKSLDLQPREVFQEDLVLTGYQVDKNKDDELTGF | |
| 80 K51A | ATGATCCCAGGTGGCCTGAGCGAAGCAAAACCAGCAACCCCAGAGATTCAAGAAATTGTTG | 1130 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTGCACATTTCCCACAGTGGCCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGAAGTTTTCCAGGAAGATTTGGTGCTGACGGGCTACCAGGTTGACAA | |
| | GAACAAAGATGACGAGCTGACGGGTTTC | |
| 80 H52A | MIPGGLSEAKPATPEIQEIVDKVKPQLEEKTGETYGKLEAVQYKTQVVRRKAFPQWPGTNY | 1131 |
| | YIKVRAGDNKYMHLKVFKSLDLQPREVFQEDLVLTGYQVDKNKDDELTGF | |
| 80 H52A | ATGATCCCAGGTGGCCTGAGCGAAGCAAAACCAGCAACCCCAGAGATTCAAGAAATTGTTG | 1132 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAAGCTTTCCCACAGTGGCCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGAAGTTTTCCAGGAAGATTTGGTGCTGACGGGCTACCAGGTTGACAA | |
| | GAACAAAGATGACGAGCTGACGGGTTTC | |
| 80 F53A | MIPGGLSEAKPATPEIQEIVDKVKPQLEEKTGETYGKLEAVQYKTQVVRRKHAPQWPGTNY | 1133 |
| | YIKVRAGDNKYMHLKVFKSLDLQPREVFQEDLVLTGYQVDKNKDDELTGF | |
| 80 F53A | ATGATCCCAGGTGGCCTGAGCGAAGCAAAACCAGCAACCCCAGAGATTCAAGAAATTGTTG | 1134 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATGCCCCACAGTGGCCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGAAGTTTTCCAGGAAGATTTGGTGCTGACGGGCTACCAGGTTGACAA | |
| | GAACAAAGATGACGAGCTGACGGGTTTC | |
| 80 P54A | MIPGGLSEAKPATPEIQEIVDKVKPQLEEKTGETYGKLEAVQYKTQVVRRKHFAQWPGTNY | 1135 |
| | YIKVRAGDNKYMHLKVFKSLDLQPREVFQEDLVLTGYQVDKNKDDELTGF | |
| 80 P54A | ATGATCCCAGGTGGCCTGAGCGAAGCAAAACCAGCAACCCCAGAGATTCAAGAAATTGTTG | 1136 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATTTCGCACAGTGGCCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGAAGTTTTCCAGGAAGATTTGGTGCTGACGGGCTACCAGGTTGACAA | |
| | GAACAAAGATGACGAGCTGACGGGTTTC | |
| 80 Q55A | MIPGGLSEAKPATPEIQEIVDKVKPQLEEKTGETYGKLEAVQYKTQVVRRKHFPAWPGTNY | 1137 |
| | YIKVRAGDNKYMHLKVFKSLDLQPREVFQEDLVLTGYQVDKNKDDELTGF | |
| 80 Q55A | ATGATCCCAGGTGGCCTGAGCGAAGCAAAACCAGCAACCCCAGAGATTCAAGAAATTGTTG | 1138 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATTTCCCAGCGTGGCCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGAAGTTTTCCAGGAAGATTTGGTGCTGACGGGCTACCAGGTTGACAA | |
| | GAACAAAGATGACGAGCTGACGGGTTTC | |
| 80 | MIPGGLSEAKPATPEIQEIVDKVKPQLEEKTGETYGKLEAVQYKTQVVRRKHFPQAPGTNY | 1139 |
| W56A | YIKVRAGDNKYMHLKVFKSLDLQPREVFQEDLVLTGYQVDKNKDDELTGF | |
| 80 | ATGATCCCAGGTGGCCTGAGCGAAGCAAAACCAGCAACCCCAGAGATTCAAGAAATTGTTG | 1140 |
| W56A | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATTTCCCACAGGCGCCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGAAGTTTTCCAGGAAGATTTGGTGCTGACGGGCTACCAGGTTGACAA | |
| | GAACAAAGATGACGAGCTGACGGGTTTC | |
| 80 P57A | MIPGGLSEAKPATPEIQEIVDKVKPQLEEKTGETYGKLEAVQYKTQVVRRKHFPQWAGTNY | 1141 |
| | YIKVRAGDNKYMHLKVFKSLDLQPREVFQEDLVLTGYQVDKNKDDELTGF | |
| 80 P57A | ATGATCCCAGGTGGCCTGAGCGAAGCAAAACCAGCAACCCCAGAGATTCAAGAAATTGTTG | 1142 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATTTCCCACAGTGGGCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGAAGTTTTCCAGGAAGATTTGGTGCTGACGGGCTACCAGGTTGACAA | |
| | GAACAAAGATGACGAGCTGACGGGTTTC | |
| 80 D82A | MIPGGLSEAKPATPEIQEIVDKVKPQLEEKTGETYGKLEAVQYKTQVVRRKHFPQWPGTNY | 1143 |
| | YIKVRAGDNKYMHLKVFKSLALQPREVFQEDLVLTGYQVDKNKDDELTGF | |
| 80 D82A | ATGATCCCAGGTGGCCTGAGCGAAGCAAAACCAGCAACCCCAGAGATTCAAGAAATTGTTG | 1144 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATTTCCCACAGTGGCCAGGTACGAACTAC | |
| | TACATCAAGGTTCGTGCGGGTGACAACAAGTATATGCACCTGAAAGTGTTTAAGAGCCTGG | |
| | CTCTGCAGCCAAGAGAAGTTTTCCAGGAAGATTTGGTGCTGACGGGCTACCAGGTTGACAA | |
| | GAACAAAGATGACGAGCTGACGGGTTTC | |
| 80 Q84A | MIPGGLSEAKPATPEIQEIVDKVKPQLEEKTGETYGKLEAVQYKTQVVRRKHFPQWPGTNY | 1145 |
| | YIKVRAGDNKYMHLKVFKSLDLAPREVFQEDLVLTGYQVDKNKDDELTGF | |
| 80 Q84A | ATGATCCCAGGTGGCCTGAGCGAAGCAAAACCAGCAACCCCAGAGATTCAAGAAATTGTTG | 1146 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATTTCCCACAGTGGCCAGGTACGAACTAC | |
| | TACATCAAGGTTCGTGCGGGTGACAACAAGTATATGCACCTGAAAGTGTTTAAGAGCCTGG | |
| | ATCTGGCGCCAAGAGAAGTTTTCCAGGAAGATTTGGTGCTGACGGGCTACCAGGTTGACAA | |
| | GAACAAAGATGACGAGCTGACGGGTTTC | |
| 80 P85A | MIPGGLSEAKPATPEIQEIVDKVKPQLEEKTGETYGKLEAVQYKTQVVRRKHFPQWPGTNY | 1147 |
| | YIKVRAGDNKYMHLKVFKSLDLQAREVFQEDLVLTGYQVDKNKDDELTGF | |
| 80 P85A | ATGATCCCAGGTGGCCTGAGCGAAGCAAAACCAGCAACCCCAGAGATTCAAGAAATTGTTG | 1148 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATTTCCCACAGTGGCCAGGTACGAACTAC | |
| | TACATCAAGGTTCGTGCGGGTGACAACAAGTATATGCACCTGAAAGTGTTTAAGAGCCTGG | |
| | ATCTGCAGGCAAGAGAAGTTTTCCAGGAAGATTTGGTGCTGACGGGCTACCAGGTTGACAA | |
| | GAACAAAGATGACGAGCTGACGGGTTTC | |
| 80 R86A | MIPGGLSEAKPATPEIQEIVDKVKPQLEEKTGETYGKLEAVQYKTQVVRRKHFPQWPGTNY | 1149 |
| | YIKVRAGDNKYMHLKVFKSLDLQPAEVFQEDLVLTGYQVDKNKDDELTGF | |
| 80 R86A | ATGATCCCAGGTGGCCTGAGCGAAGCAAAACCAGCAACCCCAGAGATTCAAGAAATTGTTG | 1150 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATTTCCCACAGTGGCCAGGTACGAACTAC | |
| | TACATCAAGGTTCGTGCGGGTGACAACAAGTATATGCACCTGAAAGTGTTTAAGAGCCTGG | |
| | ATCTGCAGCCAGCAGAAGTTTTCCAGGAAGATTTGGTGCTGACGGGCTACCAGGTTGACAA | |
| | GAACAAAGATGACGAGCTGACGGGTTTC | |
| 80 E87A | MIPGGLSEAKPATPEIQEIVDKVKPQLEEKTGETYGKLEAVQYKTQVVRRKHFPQWPGTNY | 1151 |
| | YIKVRAGDNKYMHLKVFKSLDLQPRAVFQEDLVLTGYQVDKNKDDELTGF | |
| 80 E87A | ATGATCCCAGGTGGCCTGAGCGAAGCAAAACCAGCAACCCCAGAGATTCAAGAAATTGTTG | 1152 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATTTCCCACAGTGGCCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGCAGTTTTCCAGGAAGATTTGGTGCTGACGGGCTACCAGGTTGACAA | |
| | GAACAAAGATGACGAGCTGACGGGTTTC | |
| 80 V88A | MIPGGLSEAKPATPEIQEIVDKVKPQLEEKTGETYGKLEAVQYKTQVVRRKHFPQWPGTNY | 1153 |
| | YIKVRAGDNKYMHLKVFKSLDLQPREAFQEDLVLTGYQVDKNKDDELTGF | |
| 80 V88A | ATGATCCCAGGTGGCCTGAGCGAAGCAAAACCAGCAACCCCAGAGATTCAAGAAATTGTTG | 1154 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATTTCCCACAGTGGCCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGAAGCTTTCCAGGAAGATTTGGTGCTGACGGGCTACCAGGTTGACAA | |
| | GAACAAAGATGACGAGCTGACGGGTTTC | |
| 80 F89A | MIPGGLSEAKPATPEIQEIVDKVKPQLEEKTGETYGKLEAVQYKTQVVRRKHFPQWPGTNY | 1155 |
| | YIKVRAGDNKYMHLKVFKSLDLQPREVAQEDLVLTGYQVDKNKDDELTGF | |
| 80 F89A | ATGATCCCAGGTGGCCTGAGCGAAGCAAAACCAGCAACCCCAGAGATTCAAGAAATTGTTG | 1156 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATTTCCCACAGTGGCCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGAAGTTGCCCAGGAAGATTTGGTGCTGACGGGCTACCAGGTTGACAA | |
| | GAACAAAGATGACGAGCTGACGGGTTTC | |
| 80 Q90A | MIPGGLSEAKPATPEIQEIVDKVKPQLEEKTGETYGKLEAVQYKTQVVRRKHFPQWPGTNY | 1157 |
| | YIKVRAGDNKYMHLKVFKSLDLQPREVFAEDLVLTGYQVDKNKDDELTGF | |
| 80 Q90A | ATGATCCCAGGTGGCCTGAGCGAAGCAAAACCAGCAACCCCAGAGATTCAAGAAATTGTTG | 1158 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATTTCCCACAGTGGCCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGAAGTTTTCGCGGAAGATTTGGTGCTGACGGGCTACCAGGTTGACAA | |
| | GAACAAAGATGACGAGCTGACGGGTTTC | |
| 80 V48D | MIPGGLSEAKPATPEIQEIVDKVKPQLEEKTGETYGKLEAVQYKTQVDRRKHFPQWPGTNY | 1159 |
| | YIKVRAGDNKYMHLKVFKSLDLQPREVFQEDLVLTGYQVDKNKDDELTGF | |
| 80 V48D | ATGATCCCAGGTGGCCTGAGCGAAGCAAAACCAGCAACCCCAGAGATTCAAGAAATTGTTG | 1160 |
| | ACAAAGTGAAGCCGCAGCTGGAAGAGAAAACCGGCGAAACCTACGGCAAACTGGAAGCCGT | |
| | CCAGTATAAGACTCAAGTCGATCGTCGTAAACATTTCCCACAGTGGCCAGGTACGAACTAC | |
| | TACATCAAGGTTCGTGCGGGTGACAACAAGTATATGCACCTGAAAGTGTTTAAGAGCCTGG | |
| | ATCTGCAGCCAAGAGAAGTTTTCCAGGAAGATTTGGTGCTGACGGGCTACCAGGTTGACAA | |
| | GAACAAAGATGACGAGCTGACGGGTTTC | |
| 80 L83A | MIPGGLSEAKPATPEIQEIVDKVKPQLEEKTGETYGKLEAVQYKTQVVRRKHFPQWPGTNY | 1161 |
| | YIKVRAGDNKYMHLKVFKSLDAQPREVFQEDLVLTGYQVDKNKDDELTGF | |
| 80 L83A | ATGATCCCAGGTGGCCTGAGCGAAGCAAAACCAGCAACCCCAGAGATTCAAGAAATTGTTG | 1162 |
| | ACAAAGTGAAGCCGCAGCTGGAAGAGAAAACCGGCGAAACCTACGGCAAACTGGAAGCCGT | |
| | CCAGTATAAGACTCAAGTCGTCCGTCGTAAACATTTCCCACAGTGGCCAGGTACGAACTAC | |
| | TACATCAAGGTTCGTGCGGGTGACAACAAGTATATGCACCTGAAAGTGTTTAAGAGCCTGG | |
| | ATGCGCAGCCAAGAGAAGTTTTCCAGGAAGATTTGGTGCTGACGGGCTACCAGGTTGACAA | |
| | GAACAAAGATGACGAGCTGACGGGTTTC | |
| 127 Fc | MIPGGLSEAKPATPEIQEIVDKVKPQLEEKTGETYGKLEAVQYKTQVVTQAIPWREWGTNY | 1121 |
| fusion | YIKVRAGDNKYMHLKVFKSLIPEWPKDASEDLVLTGYQVDKNKDDELTGFAAAGGGGSGGG | |
| | GSGGGGSGGGGSEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLNGKEYKCKVSN | |
| | KALPAPIEKTISKAKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALHNHYTQKSLSLSPGK | |
| 127 Fc | ATGATCCCTGGCGGCCTGTCGGAAGCCAAGCCAGCCACACCGGAGATTCAGGAAATCGTGG | 1163 |
| fusion | ATAAGGTCAAGCCCCAGCTGGAGGAAAAGACCGGCGAAACCTACGGCAAACTGGAAGCCGT | |
| | CCAGTATAAGACTCAAGTCGTCACCCAGGCAATCCCATGGCGTGAATGGGGTACGAACTAC | |
| | TACATCAAAGTTCGTGCGGGTGACAACAAGTATATGCACCTGAAAGTGTTTAAGAGCCTGA | |
| | TCCCAGAATGGCCAAAAGATGCATCTGAAGATTTGGTGCTGACGGGCTACCAGGTTGACAA | |
| | GAACAAAGATGACGAGCTGACGGGTTTCGCGGCCGCCGGTGGTGGAGGGTCCGGTGGCGGG | |
| | GGCTCAGGGGGCGGTGGCAGCGGGGGTGGCGGATCGGAACCTAAGTCCTGCGACAAGACCC | |
| | ATACTTGCCCTCCGTGCCCCGCCCCGGAACTCCTGGGAGGCCCGTCTGTGTTTCTGTTCCC | |
| | GCCAAAACCGAAGGACACCCTGATGATTTCCCGGACGCCTGAAGTGACCTGTGTGGTGGTC | |
| | GATGTGTCCCACGAGGACCCCGAAGTCAAGTTCAATTGGTACGTGGATGGAGTGGAGGTCC | |
| | ACAACGCCAAGACAAAGCCCAGGGAGGAACAGTACAACAGCACCTACCGGGTGGTGTCAGT | |
| | GCTGACTGTGCTGCACCAAGACTGGCTGAACGGAAAGGAGTACAAGTGCAAAGTGTCGAAC | |
| | AAGGCCCTGCCTGCACCCATCGAAAAGACTATCAGCAAAGCTAAGGGACAGCCCCGCGAGC | |
| | CGCAAGTCTATACCCTTCCACCTTCGCGCGAGGAGATGACCAAGAACCAAGTGTCCCTGAC | |
| | TTGCCTCGTCAAGGGATTCTACCCCTCCGACATCGCGGTGGAATGGGAGTCCAATGGCCAG | |
| | CCAGAAAACAACTACAAGACCACTCCTCCGGTGCTCGATTCGGATGGAAGCTTCTTCCTGT | |
| | ACTCCAAGTTGACGGTCGACAAGTCAAGATGGCAGCAGGGCAACGTGTTCAGCTGTTCCGT | |
| | GATGCACGAAGCCCTGCACAACCATTACACCCAGAAGTCCCTCTCCCTGTCACCGGGGAAG | |
| 80 | MIPGGLSEAKPATPEIQEIVDKVKPQLEEKTGETYGKLEAVQYKTQVVRRKHFPQWPGTNY | 1164 |
| monomer | YIKVRAGDNKYMHLKVFKSLDLQPREVFQEDLVLTGYQVDKNKDDELTGF | |
| 80 | ATGATTCCCGGCGGACTTTCGGAAGCCAAGCCAGCCACTCCGGAGATTCAGGAAATCGTGG | 1165 |
| monomer | ACAAGGTCAAACCGCAGCTGGAAGAAAAGACCGGGGAAACCTACGGGAAGTTGGAGGCGGT | |
| | CCAGTACAAAACCCAGGTCGTGCGCAGAAAACACTTCCCCCAATGGCCCGGTACCAATTAC | |
| | TATATCAAGGTTCGCGCCGGAGACAACAAGTACATGCACCTGAAAGTGTTCAAGAGCCTGG | |
| | ATCTGCAGCCTCGCGAAGTGTTCCAGGAGGACCTGGTGCTCACCGGATACCAGGTCGACAA | |
| | GAACAAGGACGACGAACTGACAGGCTTC | |
| 80 ILF | MIPGGLSEAKPATPEIQEIVDKVKPQLEEKTGETYGKLEAVQYKTQVVRRKHFPQWPGTNY | 1122 |
| | YIKVRAGDNKYMHLKVFKSLDLQPREVFQEDLVLTGYQVDKNKDDELTGFAEAAAKEAAAK | |
| | EAAAKEAAAKEAAAKEAAAKMIPGGLSEAKPATPEIQEIVDKVKPQLEEKTGETYGKLEAV | |
| | QYKTQVVRRKHFPQWPGTNYYIKVRAGDNKYMHLKVFKSLDLQPREVFQEDLVLTGYQVDK | |
| | NKDDELTGF | |
| 80 ILF | ATGATTCCGGGTGGCCTTTCCGAGGCCAAGCCTGCCACTCCCGAGATCCAGGAGATCGTGG | 1166 |
| | ATAAGGTCAAGCCACAGCTGGAGGAAAAGACTGGAGAGACTTACGGAAAGCTCGAAGCCGT | |
| | GCAGTACAAGACCCAAGTCGTGCGGCGGAAGCACTTCCCGCAATGGCCTGGAACCAACTAC | |
| | TACATCAAAGTCCGGGCCGGGGATAACAAGTATATGCATCTCAAGGTGTTCAAGTCCTTGG | |
| | ACCTCCAACCGAGGGAGGTCTTTCAGGAGGATCTGGTGCTGACGGGCTACCAAGTGGACAA | |
| | GAATAAGGACGATGAGCTGACCGGCTTCGCTGAAGCGGCAGCGAAGGAAGCCGCGGCGAAA | |
| | GAAGCCGCAGCCAAGGAGGCAGCCGCCAAAGAGGCCGCCGCTAAGGAAGCTGCCGCCAAGA | |
| | TGATTCCCGGCGGACTTTCGGAAGCCAAGCCAGCCACTCCGGAGATTCAGGAAATCGTGGA | |
| | CAAGGTCAAACCGCAGCTGGAAGAAAAGACCGGGGAAACCTACGGGAAGTTGGAGGCGGTC | |
| | CAGTACAAAACCCAGGTCGTGCGCAGAAAACACTTCCCCCAATGGCCCGGTACCAATTACT | |
| | ATATCAAGGTTCGCGCCGGAGACAACAAGTACATGCACCTGAAAGTGTTCAAGAGCCTGGA | |
| | TCTGCAGCCTCGCGAAGTGTTCCAGGAGGACCTGGTGCTCACCGGATACCAGGTCGACAAG | |
| | AACAAGGACGACGAACTGACAGGCTTC | |
| PD-L1 | MAFTVTVPKDLYVVEYGSNMTIECKFPVEKQLDLAALIVYWEMEDKNIIQFVHGEEDLKVQ | 1167 |
| domain | HSSYRQRARLLKDQLSLGNAALQITDVKLQDAGVYRCMISYGGADYKRITVKVNA | |
| PD-L1 | ATGGCGTTCACCGTCACTGTCCCGAAAGACCTGTATGTAGTGGAGTATGGTAGCAATATGA | 1168 |
| domain | CCATTGAGTGCAAGTTTCCTGTGGAGAAACAGCTGGACCTGGCGGCGTTGATTGTTTACTG | |
| | GGAAATGGAAGATAAGAATATCATTCAGTTTGTGCATGGTGAGGAAGATTTGAAAGTTCAA | |
| | CACAGCTCCTACCGCCAGCGTGCACGCCTGCTGAAAGATCAGCTGAGCCTGGGCAATGCTG | |
| | CCCTGCAAATCACTGATGTGAAGCTGCAAGATGCGGGTGTTTATCGTTGCATGATCAGCTA | |
| | CGGTGGCGCAGACTACAAGCGTATCACCGTTAAAGTCAATGCG | |

What is claimed is:

1. A protein comprising a PD-L1 binding polypeptide that binds to PD-L1 with a Kd of 1×10⁻⁶M or less, wherein the PD-L1 binding polypeptide comprises an amino acid sequence having at least 95% identity to the amino acid sequence of:
MIPGGLSEAKPATPEIQEIVDK VKPQLEEKTGETYGKLEAVQYKTQVV-(Xaa)ₙ-GTNYYIKVRAGDNKYMHLKVFKSL-(Xaa)ₘ-EDLVLTGYQVDKNKDDELTGF (SEQ ID NO: 4), wherein
Xaa, individually for each occurrence, is an amino acid residue, and
n and m are each, independently, an integer from 3-20.

2. A protein comprising a PD-L1 binding polypeptide that binds to PD-L1 with a Kd of 1×10⁻⁶M or less, wherein the PD-L1 binding polypeptide comprises an amino acid sequence having at least 95% identity to the amino acid sequence of:
MIPGGLSEAKPATPEIQEIVDK VKPQLEEKTGETYGKLEAVQYKTQVD-(Xaa)ₙ-GTNYYIKVRAGDNKYMHLKVFKSL-(Xaa)ₘ-EDLVLTGYQVDKNKDDELTGF (SEQ ID NO: 5), wherein
Xaa, individually for each occurrence, is an amino acid residue, and
n and m are each, independently, an integer from 3-20.

3. The protein of claim 1 or 2, wherein (Xaa)ₙ is an amino acid sequence selected from SEQ ID NOs: 6-259, or an amino acid sequence having at least 90% identity thereto.

4. The protein of claim 3, wherein (Xaa)ₙ is an amino acid sequence selected from SEQ ID NOs: 6-259.

5. The protein of any one of claims 1-4, wherein (Xaa)ₘ is an amino acid sequence selected from SEQ ID NOs: 260-513, or an amino acid sequence having at least 90% identity thereto.

6. The protein of claim 5, wherein (Xaa)ₘ is an amino acid sequence selected from SEQ ID NOs: 260-513.

7. The protein of any one of claims 1-7, wherein the PD-L1 binding polypeptide comprises an amino acid sequence having at least 90% identity to the amino acid sequence of any one of SEQ ID NOs: 514-767.

8. The protein of claim 7, wherein the PD-L1 binding polypeptide comprises an amino acid sequence having at least 95% identity to the amino acid sequence of any one of SEQ ID NOS: 514-767.

9. The protein of claim 8, wherein the PD-L1 binding polypeptide comprises the amino acid sequence of any one of SEQ ID NOs: 514-767.

10. The protein of any one of claims 1-9, wherein the PD-L1 binding polypeptide is encoded by a polynucleotide comprising a nucleotide sequence having at least 90% identity to the nucleotide sequence of any one of SEQ ID NOs: 768-1021, 126, 1128, 1130, 1132, 1134, 11336, 1138, 1140, 1142, 1144, 1146, 1148, 1150, 1152, 1154, 1156, 1158, 1160, 1162, 1163, 1165, 1166, and 1168.

11. A fusion protein comprising a homodimer of the protein of any one of claim 1-10.

12. A fusion protein comprising the protein of any one of claim 1-10 and a soluble receptor, a growth factor, a cytokine, a chemokine, a costimulatory agonist, or a checkpoint inhibitor.

13. A fusion protein comprising the protein of any one of claim 1-10 and a half-life extending polypeptide.

14. The fusion protein of claim 13, wherein the half-life extending polypeptide is selected from the group consisting of an Fc domain, an albumin protein, an albumin-binding polypeptide, transferrin, a transferrin-binding polypeptide, fibronectin, or a fibronectin-binding polypeptide.

15. The fusion protein of claim 14, wherein the half-life extending polypeptide is an Fc domain.

16. The fusion protein of any one of claims 11-14 further comprising a linker, optionally a flexible linker or a rigid linker.

17. The fusion protein of claim 16 (i) comprising an amino acid sequence having at least 90% identity to the amino acid sequence of SEQ ID NO: 1122 or (ii) encoded by a polynucleotide comprising a nucleotide sequence having at least 90% identity to the nucleotide sequence of SEQ ID NO: 1166.

18. The fusion protein of claim 17 (i) comprising the amino acid sequence of SEQ ID NO: 1122 or (ii) encoded by a polynucleotide comprising the nucleotide sequence of SEQ ID NO: 1166.

19. The fusion protein of claim 16 (i) comprising an amino acid sequence having at least 90% identity to the amino acid sequence of SEQ ID NO: 1121 or (ii) encoded by a polynucleotide comprising a nucleotide sequence having at least 90% identity to the nucleotide sequence of SEQ ID NO: 1163.

20. The fusion protein of claim 17 (i) comprising the amino acid sequence of SEQ ID NO: 1121 or (ii) encoded by a polynucleotide comprising the nucleotide sequence of SEQ ID NO: 1163.

21. A recombinant antibody comprising a VH and/or VL chains forming an antigen binding sites that bind to a target antigen, wherein at least one of the VH and/or VL chains is a fusion protein comprising the protein of any one of claims 1-10.

22. The recombinant antibody of claim 21, wherein the target antigen is selected from the group consisting of an immune checkpoint, an immune costimulatory receptor, an angiogenic factor, and a tumor antigen.

23. The recombinant antibody of claim 21 or 22, wherein the target antigen is selected from the group consisting of PD-1, PD-L2, CTLA-4, NKG2A, KIR, LAG-3, TIM-3, CD96, VISTA, TIGIT, CD28, ICOS, CD137, OX40, GITR, CD27, CD30, HVEM, DNAM-1 or CD28H, CEACAM-1, CEACAM-5, BTLA, LAIR1, CD160, 2B4, TGFR, B7-H3, B7-H4, CD40, CD40L, CD47, CD70, CD80, CD86, CD94, CD137, CD137L, CD226, Galectin-9, GITRL, HHLA2, ICOS, ICOSL, LIGHT, MHC class I or II, NKG2a, NKG2d, OX4OL, PVR, SIRP□, TCR, CD20, CD30, CD33, CD38, CD52, VEGF, VEGF receptors, EGFR, Her2/neu, ILT1, ILT2, ILT3, ILT4, ILT5, ILT6, ILT7, ILT8, KIR2DL1, KIR2DL2, KIR2DL3, KIR2DL4, KIR2DL5A, KIR2DL5B, KIR3DL1, KIR3DL2, KIR3DL3, NKG2A, NKG2C, NKG2E or TSLP.

24. A recombinant receptor trap fusion protein comprising (i) a ligand binding domain of a receptor, and (ii) the protein of any one of claims 1-10.

25. The recombinant receptor trap fusion protein of claim 24, wherein the ligand binding domain binds to PGE2, TGF-β, VEGF, CCL2, IDO, CSF1, IL-10, IL-13, IL-23, or adenosine.

26. A recombinant receptor ligand fusion protein comprising (i) a polypeptide ligand sequence that binds to an agonizes or antagonizes its cognate receptor, and (ii) the protein of any one of claims 1-10.

27. The recombinant receptor ligand fusion protein of claim 26, wherein the polypeptide ligand is a ligand for a co-stimulatory receptor and agonizes the co-stimulatory receptor upon binding.

28. The recombinant receptor ligand fusion protein of claim 27, wherein the polypeptide ligand is selected from B7.1, 4-1BBL, OX40L, GITRL or LIGHT.

29. The recombinant receptor ligand fusion protein of claim 27, further including a multimerization domain that induces multimerization of the recombinant receptor ligand fusion protein.

30. The recombinant receptor ligand fusion protein of claim 27, wherein the polypeptide ligand is an immunostimulatory cytokine that promotes antitumor immunity.

31. The recombinant receptor ligand fusion protein of claim 30, wherein the polypeptide ligand is selected from IFN-α2, IL-2, IL-15, IL-21, and IL-12.

32. A multispecific T-cell engaging fusion protein comprising (i) a CD3 binding polypeptide binds to CD3 on the surface of T-cells, and (ii) the protein of any one of claims 1-10.

33. A chimeric receptor fusion protein comprising (i) an extracellular portion including protein of any one of claims 1-10; (ii) a transmembrane domain; and (iii) a cytoplasmic domain comprising a 4-1BB signaling domain and a CD3& signaling domain, and optionally a costimulatory signaling region.

34. A polynucleotide comprising a nucleotide sequence encoding the protein of any one of the preceding claims.

35. The polynucleotide of claim 34 comprising a nucleotide sequence having at least 90% identity to the nucleotide sequence of any one of SEQ ID NOs: 768-1021, 1126, 1128, 1130, 1132, 1134, 11336, 1138, 1140, 1142, 1144, 1146, 1148, 1150, 1152, 1154, 1156, 1158, 1160, 1162, 1163, 1165, 1166, and 1168.

36. The polynucleotide of claim 35 comprising the nucleotide sequence of any one of SEQ ID NOs: 768-1021, 1126, 1128, 1130, 1132, 1134, 11336, 1138, 1140, 1142, 1144, 1146, 1148, 1150, 1152, 1154, 1156, 1158, 1160, 1162, 1163, 1165, 1166, and 1168.

37. A vector, optionally a viral vector or a plasmid vector, comprising the polynucleotide of any one of claims 34-36.

38. A cell, optionally a mammalian cell, comprising the polynucleotide of any one of claims 34-36 or the vector of claim 37.

39. A pharmaceutical composition comprising: (a) the protein of any one of the preceding claims, the fusion protein of any one of the preceding claims, the recombinant antibody of any one of the preceding claims, the recombinant receptor trap fusion protein of any one of the preceding claims, the recombinant receptor ligand fusion protein of any one of the preceding claims, the multispecific T-cell engaging fusion protein of any one of the preceding claims, the chimeric receptor fusion protein of any one of the preceding claims, the polynucleotide of any one of the preceding claims, the vector of any one of the preceding claims, or the cell of c of any one of the preceding claims; and (b) a pharmaceutically acceptable excipient.

40. A method comprising administering to a subject the pharmaceutical composition of claim 39.

41. The method of claim 40, wherein the subject has a cancer.

42. The method of claim 40 or 41, wherein the pharmaceutical composition is administered subcutaneously, intravenously, or intramuscularly.


SEQUENCE LISTING (1212 sequences)

SEQ ID NO: 1 - protein, 48 aa, synthetic construct
  SITE 48: note = X is V or D
MIPGGLSEAKPATPEIQEIVDKVKPQLEEKTGETYGKLEAVQYKTQVX

SEQ ID NO: 2 - protein, 24 aa, synthetic construct
GTNYYIKVRAGDNKYMHLKVFKSL

SEQ ID NO: 3 - protein, 21 aa, synthetic construct
EDLVLTGYQVDKNKDDELTGF

SEQ ID NO: 4 - protein, 95 aa, synthetic construct
  REPEAT 49: note = may repeat 3-20 times
  REPEAT 74: note = May repeat 3-20 times
MIPGGLSEAKPATPEIQEIVDKVKPQLEEKTGETYGKLEAVQYKTQVVXGTNYYIKVRAGDNKYMHLKVFKSLXEDLVLTGYQVDKNKDDELTGF

SEQ ID NO: 5 - protein, 95 aa, synthetic construct
  REPEAT 49: note = May repeat 3-20 times
  REPEAT 74: note = May repeat 3-20 times
MIPGGLSEAKPATPEIQEIVDKVKPQLEEKTGETYGKLEAVQYKTQVDXGTNYYIKVRAGDNKYMHLKVFKSLXEDLVLTGYQVDKNKDDELTGF

SEQ ID NO: 6 - protein, 9 aa, synthetic construct
VYHVRWNYL

SEQ ID NO: 7 - protein, 9 aa, synthetic construct
ASFIDIGWH

SEQ ID NO: 8 - protein, 9 aa, synthetic construct
PNQHFIVPY

SEQ ID NO: 9 - protein, 9 aa, synthetic construct
FQTWDHAWT

SEQ ID NO: 10 - protein, 9 aa, synthetic construct
FLDWKQHWR

SEQ ID NO: 11 - protein, 9 aa, synthetic construct
LSEAVYIWA

SEQ ID NO: 12 - protein, 9 aa, synthetic construct
RQYYEWSWV

SEQ ID NO: 13 - protein, 9 aa, synthetic construct
PLPDPIDWV

SEQ ID NO: 14 - protein, 9 aa, synthetic construct
QSHVEGEWL

SEQ ID NO: 15 - protein, 9 aa, synthetic construct
DDVKNGFVQ

SEQ ID NO: 16 - protein, 9 aa, synthetic construct
EVQQQENFQ

SEQ ID NO: 17 - protein, 9 aa, synthetic construct
QLKFPPYVS

SEQ ID NO: 18 - protein, 9 aa, synthetic construct
LFREEKYNY

SEQ ID NO: 19 - protein, 9 aa, synthetic construct
SFLVDPLHL

SEQ ID NO: 20 - protein, 9 aa, synthetic construct
PYFWFQHAK

SEQ ID NO: 21 - protein, 9 aa, synthetic construct
HIQWPPWVE

SEQ ID NO: 22 - protein, 9 aa, synthetic construct
IVYTIWNYV

SEQ ID NO: 23 - protein, 9 aa, synthetic construct
EAHQDLHFW

SEQ ID NO: 24 - protein, 9 aa, synthetic construct
YRFGPPYVK

SEQ ID NO: 25 - protein, 9 aa, synthetic construct
ANSKWHYAF

SEQ ID NO: 26 - protein, 9 aa, synthetic construct
KRPHFLQFS

SEQ ID NO: 27 - protein, 9 aa, synthetic construct
HRLHFPIYY

SEQ ID NO: 28 - protein, 9 aa, synthetic construct
YWEHHHYGF

SEQ ID NO: 29 - protein, 9 aa, synthetic construct
EEGTPEWAP

SEQ ID NO: 30 - protein, 9 aa, synthetic construct
VRHPRWFFI

SEQ ID NO: 31 - protein, 9 aa, synthetic construct
FVFLEVKWD

SEQ ID NO: 32 - protein, 9 aa, synthetic construct
DVKPKWQWT

SEQ ID NO: 33 - protein, 9 aa, synthetic construct
AAFSVHPSS

SEQ ID NO: 34 - protein, 9 aa, synthetic construct
FTFVDIHWY

SEQ ID NO: 35 - protein, 9 aa, synthetic construct
KYVVQASKV

SEQ ID NO: 36 - protein, 9 aa, synthetic construct
AQDLSAFQI

SEQ ID NO: 37 - protein, 9 aa, synthetic construct
KYVVQASKV

SEQ ID NO: 38 - protein, 9 aa, synthetic construct
NQPQPPWIR

SEQ ID NO: 39 - protein, 9 aa, synthetic construct
THLNNNAWT

SEQ ID NO: 40 - protein, 9 aa, synthetic construct
KYETHFYYE

SEQ ID NO: 41 - protein, 9 aa, synthetic construct
WSFAQVGWH

SEQ ID NO: 42 - protein, 9 aa, synthetic construct
HHFAEVKWY

SEQ ID NO: 43 - protein, 9 aa, synthetic construct
RVPLDSNKW

SEQ ID NO: 44 - protein, 9 aa, synthetic construct
EIRPVWDWS

SEQ ID NO: 45 - protein, 9 aa, synthetic construct
KRRHFPQWQ

SEQ ID NO: 46 - protein, 9 aa, synthetic construct
GTRDEWHWV

SEQ ID NO: 47 - protein, 9 aa, synthetic construct
IQHIWWDFV

SEQ ID NO: 48 - protein, 9 aa, synthetic construct
VQHPQWHWK

SEQ ID NO: 49 - protein, 9 aa, synthetic construct
RTKETKKSW

SEQ ID NO: 50 - protein, 9 aa, synthetic construct
REYPHFQGY

SEQ ID NO: 51 - protein, 9 aa, synthetic construct
VQHSLWYFQ

SEQ ID NO: 52 - protein, 9 aa, synthetic construct
GVKQTWNWN

SEQ ID NO: 53 - protein, 9 aa, synthetic construct
TRKHFPQYW

SEQ ID NO: 54 - protein, 9 aa, synthetic construct
KYYAIFDYK

SEQ ID NO: 55 - protein, 9 aa, synthetic construct
GRQVKWAWT

SEQ ID NO: 56 - protein, 9 aa, synthetic construct
SHHASPSHI

SEQ ID NO: 57 - protein, 9 aa, synthetic construct
GIREDWNWS

SEQ ID NO: 58 - protein, 9 aa, synthetic construct
AWDWLPAYR

SEQ ID NO: 59 - protein, 9 aa, synthetic construct
INQQQIKHS

SEQ ID NO: 60 - protein, 9 aa, synthetic construct
AGWSLPPHL

SEQ ID NO: 61 - protein, 9 aa, synthetic construct
GGRHRWNWS

SEQ ID NO: 62 - protein, 9 aa, synthetic construct
ELDGDHTWL

SEQ ID NO: 63 - protein, 9 aa, synthetic construct
KQLIIWHWT

SEQ ID NO: 64 - protein, 9 aa, synthetic construct
REPALWHFR

SEQ ID NO: 65 - protein, 9 aa, synthetic construct
PQVQEPWWI

SEQ ID NO: 66 - protein, 9 aa, synthetic construct
PTGTHQWAA

SEQ ID NO: 67 - protein, 9 aa, synthetic construct
FRQHFPNFS

SEQ ID NO: 68 - protein, 9 aa, synthetic construct
QEYEDQEWA

SEQ ID NO: 69 - protein, 9 aa, synthetic construct
IAHIHWKYF

SEQ ID NO: 70 - protein, 9 aa, synthetic construct
DYRAIWHWW

SEQ ID NO: 71 - protein, 9 aa, synthetic construct
NLKPDVQGS

SEQ ID NO: 72 - protein, 9 aa, synthetic construct
STKNLWRWS

SEQ ID NO: 73 - protein, 9 aa, synthetic construct
RVQGLWNFQ

SEQ ID NO: 74 - protein, 9 aa, synthetic construct
YKVDNNNYQ

SEQ ID NO: 75 - protein, 9 aa, synthetic construct
IEEPSAFWW

SEQ ID NO: 76 - protein, 9 aa, synthetic construct
ASFPEISWY

SEQ ID NO: 77 - protein, 9 aa, synthetic construct
IHEPEIWWY

SEQ ID NO: 78 - protein, 9 aa, synthetic construct
DWGWGIPWV

SEQ ID NO: 79 - protein, 9 aa, synthetic construct
RRWELWSFQ

SEQ ID NO: 80 - protein, 9 aa, synthetic construct
INQHENFKQ

SEQ ID NO: 81 - protein, 9 aa, synthetic construct
HDFTIRYPW

SEQ ID NO: 82 - protein, 9 aa, synthetic construct
WDRPRWGWT

SEQ ID NO: 83 - protein, 9 aa, synthetic construct
DQGWWENWA

SEQ ID NO: 84 - protein, 9 aa, synthetic construct
IWHSEWSYT

SEQ ID NO: 85 - protein, 9 aa, synthetic construct
RRKHFPQWP

SEQ ID NO: 86 - protein, 9 aa, synthetic construct
KAYVIWSYK

SEQ ID NO: 87 - protein, 9 aa, synthetic construct
FWQLHRYGF

SEQ ID NO: 88 - protein, 9 aa, synthetic construct
IYHELWNFY

SEQ ID NO: 89 - protein, 9 aa, synthetic construct
LIRHLWSWS

SEQ ID NO: 90 - protein, 9 aa, synthetic construct
NDGKSLVLA

SEQ ID NO: 91 - protein, 9 aa, synthetic construct
DVRGQWAWT

SEQ ID NO: 92 - protein, 9 aa, synthetic construct
KRLHLPQYP

SEQ ID NO: 93 - protein, 9 aa, synthetic construct
AVHAPQYAP

SEQ ID NO: 94 - protein, 9 aa, synthetic construct
DFRVAWHWV

SEQ ID NO: 95 - protein, 9 aa, synthetic construct
LGWWFGINQ

SEQ ID NO: 96 - protein, 9 aa, synthetic construct
NQEHFRRFI

SEQ ID NO: 97 - protein, 8 aa, synthetic construct
DIDDDLWS

SEQ ID NO: 98 - protein, 9 aa, synthetic construct
QWGVDLVYT

SEQ ID NO: 99 - protein, 9 aa, synthetic construct
KVYLVWAWT

SEQ ID NO: 100 - protein, 9 aa, synthetic construct
TIHPQAKYW

SEQ ID NO: 101 - protein, 8 aa, synthetic construct
FNQSRHGH

SEQ ID NO: 102 - protein, 9 aa, synthetic construct
ELDDAVKYW

SEQ ID NO: 103 - protein, 10 aa, synthetic construct
DWAIWRDKKV

SEQ ID NO: 104 - protein, 9 aa, synthetic construct
TWGERLHYP

SEQ ID NO: 105 - protein, 9 aa, synthetic construct
HTDVDWHFW

SEQ ID NO: 106 - protein, 9 aa, synthetic construct
HHHRFAKWW

SEQ ID NO: 107 - protein, 9 aa, synthetic construct
RKAAVVARH

SEQ ID NO: 108 - protein, 10 aa, synthetic construct
DFSFPLVHWY

SEQ ID NO: 109 - protein, 10 aa, synthetic construct
DNAHPLVKNW

SEQ ID NO: 110 - protein, 9 aa, synthetic construct
ETSENVIQW

SEQ ID NO: 111 - protein, 9 aa, synthetic construct
FSDLEWEGQ

SEQ ID NO: 112 - protein, 9 aa, synthetic construct
FTDIHWSTW

SEQ ID NO: 113 - protein, 9 aa, synthetic construct
REHASVKFW

SEQ ID NO: 114 - protein, 9 aa, synthetic construct
ISGVGLVPN

SEQ ID NO: 115 - protein, 9 aa, synthetic construct
HTQPKALDW

SEQ ID NO: 116 - protein, 9 aa, synthetic construct
IQDRLGGFI

SEQ ID NO: 117 - protein, 9 aa, synthetic construct
RFYHQWHFL

SEQ ID NO: 118 - protein, 10 aa, synthetic construct
DWVTRHHTLV

SEQ ID NO: 119 - protein, 9 aa, synthetic construct
QSSDLAKLW

SEQ ID NO: 120 - protein, 9 aa, synthetic construct
RFYHQWHFL

SEQ ID NO: 121 - protein, 10 aa, synthetic construct
DWVTRHHTLV

SEQ ID NO: 122 - protein, 9 aa, synthetic construct
HEVRKTYEF

SEQ ID NO: 123 - protein, 9 aa, synthetic construct
VISIPLVWN

SEQ ID NO: 124 - protein, 9 aa, synthetic construct
SWVFKPFYH

SEQ ID NO: 125 - protein, 9 aa, synthetic construct
PEVSITRWQ

SEQ ID NO: 126 - protein, 9 aa, synthetic construct
GNFAHERWQ

SEQ ID NO: 127 - protein, 9 aa, synthetic construct
VIHIPHQWH

SEQ ID NO: 128 - protein, 9 aa, synthetic construct
NLVYYQRDW

SEQ ID NO: 129 - protein, 9 aa, synthetic construct
QVLWEIFEH

SEQ ID NO: 130 - protein, 9 aa, synthetic construct
TRLHFPQYQ

SEQ ID NO: 131 - protein, 9 aa, synthetic construct
YEPHHPYPW

SEQ ID NO: 132 - protein, 9 aa, synthetic construct
TQAIPWREW

SEQ ID NO: 133 - protein, 9 aa, synthetic construct
PEVDKAEWW

SEQ ID NO: 134 - protein, 9 aa, synthetic construct
QRVHFPDWP

SEQ ID NO: 135 - protein, 9 aa, synthetic construct
PQPEWNEWG

SEQ ID NO: 136 - protein, 9 aa, synthetic construct
NQWEWPVDL

SEQ ID NO: 137 - protein, 9 aa, synthetic construct
ERYHFPQFT

SEQ ID NO: 138 - protein, 9 aa, synthetic construct
LYSESRDYK

SEQ ID NO: 139 - protein, 9 aa, synthetic construct
FWFEDIQWL

SEQ ID NO: 140 - protein, 8 aa, synthetic construct
AVYPTFKY

SEQ ID NO: 141 - protein, 9 aa, synthetic construct
VLRWQWGYL

SEQ ID NO: 142 - protein, 9 aa, synthetic construct
LWPLQPKNW

SEQ ID NO: 143 - protein, 9 aa, synthetic construct
DNVRHGWLS

SEQ ID NO: 144 - protein, 9 aa, synthetic construct
GLIQPPYAQ

SEQ ID NO: 145 - protein, 9 aa, synthetic construct
RQVGVWAYE

SEQ ID NO: 146 - protein, 9 aa, synthetic construct
DLHTLKSGV

SEQ ID NO: 147 - protein, 9 aa, synthetic construct
SWYKLWSYS

SEQ ID NO: 148 - protein, 9 aa, synthetic construct
DWPSHAKFW

SEQ ID NO: 149 - protein, 9 aa, synthetic construct
AYQSPPYHF

SEQ ID NO: 150 - protein, 9 aa, synthetic construct
YKNVGPWVN

SEQ ID NO: 151 - protein, 9 aa, synthetic construct
SKRSAWHWT

SEQ ID NO: 152 - protein, 8 aa, synthetic construct
VHITVEWE

SEQ ID NO: 153 - protein, 9 aa, synthetic construct
RDYDPFPYT

SEQ ID NO: 154 - protein, 9 aa, synthetic construct
KTLGVWHWS

SEQ ID NO: 155 - protein, 9 aa, synthetic construct
REVGLWRFQ

SEQ ID NO: 156 - protein, 9 aa, synthetic construct
WEEVNWRNW

SEQ ID NO: 157 - protein, 9 aa, synthetic construct
RAQLRPWVA

SEQ ID NO: 158 - protein, 9 aa, synthetic construct
YPWSWNGHY

SEQ ID NO: 159 - protein, 9 aa, synthetic construct
PQIGAPYVK

SEQ ID NO: 160 - protein, 9 aa, synthetic construct
WRTGQKWQV

SEQ ID NO: 161 - protein, 9 aa, synthetic construct
IQQLYKQTW

SEQ ID NO: 162 - protein, 9 aa, synthetic construct
IYYPIWDFH

SEQ ID NO: 163 - protein, 9 aa, synthetic construct
DNVANIVPH

SEQ ID NO: 164 - protein, 9 aa, synthetic construct
HVHWAWNYL

SEQ ID NO: 165 - protein, 9 aa, synthetic construct
GPWYFGSAW

SEQ ID NO: 166 - protein, 9 aa, synthetic construct
VLERPPYVK

SEQ ID NO: 167 - protein, 9 aa, synthetic construct
FTFSSIGWW

SEQ ID NO: 168 - protein, 9 aa, synthetic construct
ISASEIRWY

SEQ ID NO: 169 - protein, 9 aa, synthetic construct
RIWGFWAFQ

SEQ ID NO: 170 - protein, 9 aa, synthetic construct
NEAESSRFL

SEQ ID NO: 171 - protein, 9 aa, synthetic construct
VAGLKWHWE

SEQ ID NO: 172 - protein, 9 aa, synthetic construct
YRNKQWYNE

SEQ ID NO: 173 - protein, 9 aa, synthetic construct
WFDTWPVPE

SEQ ID NO: 174 - protein, 9 aa, synthetic construct
YANIGPWLY

SEQ ID NO: 175 - protein, 9 aa, synthetic construct
HQFQEIKWF

SEQ ID NO: 176 - protein, 9 aa, synthetic construct
IDHQNHPTE

SEQ ID NO: 177 - protein, 9 aa, synthetic construct
SVFQYTYKF

SEQ ID NO: 178 - protein, 9 aa, synthetic construct
DWPSHAKFW

SEQ ID NO: 179 - protein, 9 aa, synthetic construct
RQVTIWHFE

SEQ ID NO: 180 - protein, 9 aa, synthetic construct
WRRWFPQWP

SEQ ID NO: 181 - protein, 9 aa, synthetic construct
FWYPEIGWL

SEQ ID NO: 182 - protein, 9 aa, synthetic construct
ISEDSWWPH

SEQ ID NO: 183 - protein, 9 aa, synthetic construct
DHLPHAKYW

SEQ ID NO: 184 - protein, 9 aa, synthetic construct
FKTDDARWI

SEQ ID NO: 185 - protein, 9 aa, synthetic construct
LQGSRLPIE

SEQ ID NO: 186 - protein, 9 aa, synthetic construct
KAYKVFNYI

SEQ ID NO: 187 - protein, 9 aa, synthetic construct
LKTNPSPSD

SEQ ID NO: 188 - protein, 9 aa, synthetic construct
RQTDSVKWW

SEQ ID NO: 189 - protein, 9 aa, synthetic construct
HRKHFHQFT

SEQ ID NO: 190 - protein, 9 aa, synthetic construct
VVLFASGWE

SEQ ID NO: 191 - protein, 9 aa, synthetic construct
YDNVKEYPW

SEQ ID NO: 192 - protein, 9 aa, synthetic construct
IQDWTVGWE

SEQ ID NO: 193 - protein, 9 aa, synthetic construct
PQENAPWVL

SEQ ID NO: 194 - protein, 9 aa, synthetic construct
FQSTDSFWH

SEQ ID NO: 195 - protein, 9 aa, synthetic construct
GVKSTFHWS

SEQ ID NO: 196 - protein, 9 aa, synthetic construct
LRYHFPEFH

SEQ ID NO: 197 - protein, 9 aa, synthetic construct
TENNSWWPI

SEQ ID NO: 198 - protein, 9 aa, synthetic construct
LRHPVQDDD

SEQ ID NO: 199 - protein, 9 aa, synthetic construct
EERRIWNWT

SEQ ID NO: 200 - protein, 9 aa, synthetic construct
VGVGRDFPK

SEQ ID NO: 201 - protein, 9 aa, synthetic construct
LLWTPPYVD

SEQ ID NO: 202 - protein, 9 aa, synthetic construct
WKPQDSVNW

SEQ ID NO: 203 - protein, 12 aa, synthetic construct
SSPLHWELWHWS

SEQ ID NO: 204 - protein, 9 aa, synthetic construct
RDHELVNQW

SEQ ID NO: 205 - protein, 9 aa, synthetic construct
YGPHHQTYA

SEQ ID NO: 206 - protein, 9 aa, synthetic construct
QRAHFKQWY

SEQ ID NO: 207 - protein, 9 aa, synthetic construct
IRHVIWSYS

SEQ ID NO: 208 - protein, 9 aa, synthetic construct
YHGPPKEWL

SEQ ID NO: 209 - protein, 9 aa, synthetic construct
SLRPHEQLY

SEQ ID NO: 210 - protein, 9 aa, synthetic construct
VFLSEIHWY

SEQ ID NO: 211 - protein, 9 aa, synthetic construct
RADVNVIWW

SEQ ID NO: 212 - protein, 9 aa, synthetic construct
DTKTPWLWT

SEQ ID NO: 213 - protein, 9 aa, synthetic construct
QWKKTQYFF

SEQ ID NO: 214 - protein, 9 aa, synthetic construct
IAKEENHWR

SEQ ID NO: 215 - protein, 9 aa, synthetic construct
WEYAGDYLE

SEQ ID NO: 216 - protein, 9 aa, synthetic construct
THPEDAYSW

SEQ ID NO: 217 - protein, 9 aa, synthetic construct
YAFITPVTH

SEQ ID NO: 218 - protein, 9 aa, synthetic construct
KQHTKEWAP

SEQ ID NO: 219 - protein, 9 aa, synthetic construct
IKIHHGSWE

SEQ ID NO: 220 - protein, 9 aa, synthetic construct
RRVFFPEFP

SEQ ID NO: 221 - protein, 9 aa, synthetic construct
FTEVKPFVE

SEQ ID NO: 222 - protein, 9 aa, synthetic construct
KKYVIFFYE

SEQ ID NO: 223 - protein, 9 aa, synthetic construct
WRVYFPDFK

SEQ ID NO: 224 - protein, 9 aa, synthetic construct
PLTIPPFVE

SEQ ID NO: 225 - protein, 9 aa, synthetic construct
NRWNIREFE

SEQ ID NO: 226 - protein, 9 aa, synthetic construct
IQDVDWRLW

SEQ ID NO: 227 - protein, 9 aa, synthetic construct
QRDHFPEFP

SEQ ID NO: 228 - protein, 9 aa, synthetic construct
FPWDQENYY

SEQ ID NO: 229 - protein, 9 aa, synthetic construct
TRRHFVQFT

SEQ ID NO: 230 - protein, 9 aa, synthetic construct
DLHTPHYAP

SEQ ID NO: 231 - protein, 9 aa, synthetic construct
YKHDDEYGF

SEQ ID NO: 232 - protein, 9 aa, synthetic construct
YVRWHEYGF

SEQ ID NO: 233 - protein, 9 aa, synthetic construct
VWTSDDAWQ

SEQ ID NO: 234 - protein, 9 aa, synthetic construct
HSSSKPYIH

SEQ ID NO: 235 - protein, 9 aa, synthetic construct
HVSNFIIPH

SEQ ID NO: 236 - protein, 9 aa, synthetic construct
ARWHWFHHF

SEQ ID NO: 237 - protein, 9 aa, synthetic construct
TKHSATFFH

SEQ ID NO: 238 - protein, 9 aa, synthetic construct
LRYHFQHFP

SEQ ID NO: 239 - protein, 9 aa, synthetic construct
RNSGVWEFE

SEQ ID NO: 240 - protein, 9 aa, synthetic construct
WNPPSLPIS

SEQ ID NO: 241 - protein, 9 aa, synthetic construct
RDGTFWDFW

SEQ ID NO: 242 - protein, 9 aa, synthetic construct
VAGLKWHWE

SEQ ID NO: 243 - protein, 9 aa, synthetic construct
RHKGLWQFE

SEQ ID NO: 244 - protein, 9 aa, synthetic construct
LTIPPTAFS

SEQ ID NO: 245 - protein, 9 aa, synthetic construct
LLYQAPFVQ

SEQ ID NO: 246 - protein, 9 aa, synthetic construct
SRLHFPNFQ

SEQ ID NO: 247 - protein, 9 aa, synthetic construct
VEWSESGYD

SEQ ID NO: 248 - protein, 9 aa, synthetic construct
WKQITLPVD

SEQ ID NO: 249 - protein, 9 aa, synthetic construct
WDSEALPID

SEQ ID NO: 250 - protein, 9 aa, synthetic construct
GYLELWKWS

SEQ ID NO: 251 - protein, 9 aa, synthetic construct
ERFHFPQFP

SEQ ID NO: 252 - protein, 9 aa, synthetic construct
RREHFPRWP

SEQ ID NO: 253 - protein, 9 aa, synthetic construct
LQLPPRPHD

SEQ ID NO: 254 - protein, 9 aa, synthetic construct
IYYPIWGFQ

SEQ ID NO: 255 - protein, 9 aa, synthetic construct
RDPDVFVFS

SEQ ID NO: 256 - protein, 9 aa, synthetic construct
ATDQDWRKW

SEQ ID NO: 257 - protein, 10 aa, synthetic construct
AVRPIFKWAG

SEQ ID NO: 258 - protein, 9 aa, synthetic construct
TRPHFPGWN

SEQ ID NO: 259 - protein, 9 aa, synthetic construct
ISDVSVGWE

SEQ ID NO: 260 - protein, 9 aa, synthetic construct
SLEEPPVET

SEQ ID NO: 261 - protein, 9 aa, synthetic construct
REGKHGRNK

SEQ ID NO: 262 - protein, 9 aa, synthetic construct
HWNGGYHGH

SEQ ID NO: 263 - protein, 9 aa, synthetic construct
FAAETQQYY

SEQ ID NO: 264 - protein, 8 aa, synthetic construct
FNDKHNST

SEQ ID NO: 265 - protein, 9 aa, synthetic construct
NSRWYGPLG

SEQ ID NO: 266 - protein, 9 aa, synthetic construct
FESPTEDND

SEQ ID NO: 267 - protein, 9 aa, synthetic construct
HIHLNQIYR

SEQ ID NO: 268 - protein, 9 aa, synthetic construct
FFAIHRLGI

SEQ ID NO: 269 - protein, 9 aa, synthetic construct
IDRHVYWLE

SEQ ID NO: 270 - protein, 9 aa, synthetic construct
HIYEHIVYR

SEQ ID NO: 271 - protein, 9 aa, synthetic construct
GWDDKPEWD

SEQ ID NO: 272 - protein, 9 aa, synthetic construct
AWGEVVSNA

SEQ ID NO: 273 - protein, 9 aa, synthetic construct
RSFGGEWGW

SEQ ID NO: 274 - protein, 9 aa, synthetic construct
WQSLLDKNE

SEQ ID NO: 275 - protein, 9 aa, synthetic construct
GWTSDNSNF

SEQ ID NO: 276 - protein, 9 aa, synthetic construct
TPQTETEHE

SEQ ID NO: 277 - protein, 15 aa, synthetic construct
LTEHNKKHQERHLDH

SEQ ID NO: 278 - protein, 9 aa, synthetic construct
GWNAFSSPA

SEQ ID NO: 279 - protein, 9 aa, synthetic construct
DNAIYRGPT

SEQ ID NO: 280 - protein, 9 aa, synthetic construct
NIGPAKKQL

SEQ ID NO: 281 - protein, 9 aa, synthetic construct
HFTLPTQHP

SEQ ID NO: 282 - protein, 9 aa, synthetic construct
AWPEIDPPV

SEQ ID NO: 283 - protein, 9 aa, synthetic construct
WSDVLYNDF

SEQ ID NO: 284 - protein, 9 aa, synthetic construct
KKHGDEVTL

SEQ ID NO: 285 - protein, 9 aa, synthetic construct
RKERHQFDI

SEQ ID NO: 286 - protein, 9 aa, synthetic construct
FEGEDRDAV

SEQ ID NO: 287 - protein, 9 aa, synthetic construct
EKYHNIWYV

SEQ ID NO: 288 - protein, 9 aa, synthetic construct
RKPKDRQYT

SEQ ID NO: 289 - protein, 9 aa, synthetic construct
YGQKDGQKE

SEQ ID NO: 290 - protein, 9 aa, synthetic construct
IFAPLKPWR

SEQ ID NO: 291 - protein, 9 aa, synthetic construct
YGQKDGQKE

SEQ ID NO: 292 - protein, 9 aa, synthetic construct
NWNSPNLTW

SEQ ID NO: 293 - protein, 9 aa, synthetic construct
FFHNGKIYR

SEQ ID NO: 294 - protein, 9 aa, synthetic construct
PKRWAYNAY

SEQ ID NO: 295 - protein, 9 aa, synthetic construct
RRFEHLPFY

SEQ ID NO: 296 - protein, 9 aa, synthetic construct
KITGSTLTF

SEQ ID NO: 297 - protein, 9 aa, synthetic construct
FRVRDYSFL

SEQ ID NO: 298 - protein, 9 aa, synthetic construct
FDHESKPTA

SEQ ID NO: 299 - protein, 9 aa, synthetic construct
GDHLNQYPN

SEQ ID NO: 300 - protein, 9 aa, synthetic construct
FVKDDGRVD

SEQ ID NO: 301 - protein, 9 aa, synthetic construct
SRLEPAGHQ

SEQ ID NO: 302 - protein, 9 aa, synthetic construct
VQQRDVWIN

SEQ ID NO: 303 - protein, 9 aa, synthetic construct
QKIHAIFYV

SEQ ID NO: 304 - protein, 9 aa, synthetic construct
ILNAGGIRW

SEQ ID NO: 305 - protein, 9 aa, synthetic construct
FKHPEENPV

SEQ ID NO: 306 - protein, 9 aa, synthetic construct
FGLNDDEPV

SEQ ID NO: 307 - protein, 9 aa, synthetic construct
NEPLPVTNI

SEQ ID NO: 308 - protein, 9 aa, synthetic construct
FKEKVRNSI

SEQ ID NO: 309 - protein, 9 aa, synthetic construct
FKQHHNISE

SEQ ID NO: 310 - protein, 9 aa, synthetic construct
TTDPNWDVH

SEQ ID NO: 311 - protein, 9 aa, synthetic construct
FEPKKLEPA

SEQ ID NO: 312 - protein, 9 aa, synthetic construct
FKFQNESFA

SEQ ID NO: 313 - protein, 9 aa, synthetic construct
WQHIPPDFW

SEQ ID NO: 314 - protein, 9 aa, synthetic construct
INHAGQKSD

SEQ ID NO: 315 - protein, 9 aa, synthetic construct
FEPSGLAAE

SEQ ID NO: 316 - protein, 9 aa, synthetic construct
RQLHRIWYF

SEQ ID NO: 317 - protein, 9 aa, synthetic construct
FKNSNSHLE

SEQ ID NO: 318 - protein, 9 aa, synthetic construct
TFKVDTWKV

SEQ ID NO: 319 - protein, 9 aa, synthetic construct
YKAYIQGWN

SEQ ID NO: 320 - protein, 9 aa, synthetic construct
WASLESGQN

SEQ ID NO: 321 - protein, 9 aa, synthetic construct
NGLEDPTQD

SEQ ID NO: 322 - protein, 9 aa, synthetic construct
HLWNQQIYR

SEQ ID NO: 323 - protein, 9 aa, synthetic construct
HLAKGQWRE

SEQ ID NO: 324 - protein, 9 aa, synthetic construct
FHAEGDIWD

SEQ ID NO: 325 - protein, 9 aa, synthetic construct
IVVKRYWYV

SEQ ID NO: 326 - protein, 9 aa, synthetic construct
FNGAGGPDS

SEQ ID NO: 327 - protein, 9 aa, synthetic construct
GHHGDHEKW

SEQ ID NO: 328 - protein, 9 aa, synthetic construct
ITWQLNQPR

SEQ ID NO: 329 - protein, 9 aa, synthetic construct
LRVRHSAYR

SEQ ID NO: 330 - protein, 9 aa, synthetic construct
KNYPKTGVT

SEQ ID NO: 331 - protein, 9 aa, synthetic construct
HKADRQKER

SEQ ID NO: 332 - protein, 9 aa, synthetic construct
WLYGVKRLP

SEQ ID NO: 333 - protein, 9 aa, synthetic construct
WQEHTDSVE

SEQ ID NO: 334 - protein, 9 aa, synthetic construct
HLFHGLWYI

SEQ ID NO: 335 - protein, 9 aa, synthetic construct
EYHVTKRDQ

SEQ ID NO: 336 - protein, 9 aa, synthetic construct
FKESSRNDE

SEQ ID NO: 337 - protein, 9 aa, synthetic construct
IWENNLRIH

SEQ ID NO: 338 - protein, 9 aa, synthetic construct
FTRHSEPWE

SEQ ID NO: 339 - protein, 9 aa, synthetic construct
DLQPREVFQ

SEQ ID NO: 340 - protein, 9 aa, synthetic construct
FYGEPRGKG

SEQ ID NO: 341 - protein, 9 aa, synthetic construct
EWNRLSPLW

SEQ ID NO: 342 - protein, 9 aa, synthetic construct
DNNGDGNWQ

SEQ ID NO: 343 - protein, 9 aa, synthetic construct
FEGENHQID

SEQ ID NO: 344 - protein, 9 aa, synthetic construct
RLWDDRVSG

SEQ ID NO: 345 - protein, 9 aa, synthetic construct
FQPRTKATE

SEQ ID NO: 346 - protein, 9 aa, synthetic construct
GNIEPITDL

SEQ ID NO: 347 - protein, 9 aa, synthetic construct
WSEKVAVFP

SEQ ID NO: 348 - protein, 9 aa, synthetic construct
FNHRPGKPD

SEQ ID NO: 349 - protein, 9 aa, synthetic construct
AHLQYEWGG

SEQ ID NO: 350 - protein, 9 aa, synthetic construct
NPWLHPQDF

SEQ ID NO: 351 - protein, 9 aa, synthetic construct
ILWPTKPER

SEQ ID NO: 352 - protein, 9 aa, synthetic construct
IVKRAVILW

SEQ ID NO: 353 - protein, 9 aa, synthetic construct
FQKGSNKPE

SEQ ID NO: 354 - protein, 9 aa, synthetic construct
WKNLLKPIH

SEQ ID NO: 355 - protein, 9 aa, synthetic construct
ELLHGLWWV

SEQ ID NO: 356 - protein, 9 aa, synthetic construct
WWKTISADF

SEQ ID NO: 357 - protein, 9 aa, synthetic construct
PIFEIVREG

SEQ ID NO: 358 - protein, 9 aa, synthetic construct
WHKPKDNII

SEQ ID NO: 359 - protein, 9 aa, synthetic construct
APAAPPWEF

SEQ ID NO: 360 - protein, 9 aa, synthetic construct
WFNENREDT

SEQ ID NO: 361 - protein, 9 aa, synthetic construct
WPFLVIEKY

SEQ ID NO: 362 - protein, 9 aa, synthetic construct
RFRDGGHHV

SEQ ID NO: 363 - protein, 9 aa, synthetic construct
WRNDTSSIY

SEQ ID NO: 364 - protein, 9 aa, synthetic construct
WPADILTQH

SEQ ID NO: 365 - protein, 9 aa, synthetic construct
EKKWGWIYI

SEQ ID NO: 366 - protein, 9 aa, synthetic construct
IPAWQQIHV

SEQ ID NO: 367 - protein, 9 aa, synthetic construct
WLEATPHTQ

SEQ ID NO: 368 - protein, 9 aa, synthetic construct
NWNYPQQPF

SEQ ID NO: 369 - protein, 9 aa, synthetic construct
WGSILTDGP

SEQ ID NO: 370 - protein, 9 aa, synthetic construct
HFYRGILYR

SEQ ID NO: 371 - protein, 9 aa, synthetic construct
DLRAPRNDN

SEQ ID NO: 372 - protein, 8 aa, synthetic construct
PPFKHWHL

SEQ ID NO: 373 - protein, 9 aa, synthetic construct
WNLNSDKSS

SEQ ID NO: 374 - protein, 9 aa, synthetic construct
DLRAPRNDN

SEQ ID NO: 375 - protein, 8 aa, synthetic construct
PPFKHWHL

SEQ ID NO: 376 - protein, 9 aa, synthetic construct
WFGGWPLAQ

SEQ ID NO: 377 - protein, 9 aa, synthetic construct
IFQEYYPHR

SEQ ID NO: 378 - protein, 9 aa, synthetic construct
SGPEFFSRH

SEQ ID NO: 379 - protein, 9 aa, synthetic construct
YETHGNIIT

SEQ ID NO: 380 - protein, 9 aa, synthetic construct
YIARDFDWT

SEQ ID NO: 381 - protein, 9 aa, synthetic construct
IFHEPKPPR

SEQ ID NO: 382 - protein, 9 aa, synthetic construct
EPGRWRWPK

SEQ ID NO: 383 - protein, 9 aa, synthetic construct
FHQWSPALH

SEQ ID NO: 384 - protein, 9 aa, synthetic construct
EIEVNPYAN

SEQ ID NO: 385 - protein, 9 aa, synthetic construct
QYHPPYERF

SEQ ID NO: 386 - protein, 9 aa, synthetic construct
IPEWPKDAS

SEQ ID NO: 387 - protein, 9 aa, synthetic construct
IRYGLRHER

SEQ ID NO: 388 - protein, 9 aa, synthetic construct
NKENQDEED

SEQ ID NO: 389 - protein, 9 aa, synthetic construct
IFWNEKLIR

SEQ ID NO: 390 - protein, 9 aa, synthetic construct
KVYRTHHIF

SEQ ID NO: 391 - protein, 9 aa, synthetic construct
NHDQVRWNL

SEQ ID NO: 392 - protein, 9 aa, synthetic construct
HAYKGSIYR

SEQ ID NO: 393 - protein, 9 aa, synthetic construct
RFREDVDIR

SEQ ID NO: 394 - protein, 9 aa, synthetic construct
KVEYLDFIT

SEQ ID NO: 395 - protein, 8 aa, synthetic construct
QLLYEDND

SEQ ID NO: 396 - protein, 9 aa, synthetic construct
GWNYLEAIA

SEQ ID NO: 397 - protein, 9 aa, synthetic construct
FALLWSAYG

SEQ ID NO: 398 - protein, 9 aa, synthetic construct
GWQYIDLFH

SEQ ID NO: 399 - protein, 9 aa, synthetic construct
GRTVEGGRW

SEQ ID NO: 400 - protein, 9 aa, synthetic construct
YIKHHLWWI

SEQ ID NO: 401 - protein, 9 aa, synthetic construct
FEGRHKDPE

SEQ ID NO: 402 - protein, 9 aa, synthetic construct
FLPDKDDPF

SEQ ID NO: 403 - protein, 9 aa, synthetic construct
RQIAGYIAI

SEQ ID NO: 404 - protein, 9 aa, synthetic construct
YRTNRGHER

SEQ ID NO: 405 - protein, 9 aa, synthetic construct
FWNAPDDRQ

SEQ ID NO: 406 - protein, 9 aa, synthetic construct
IYQRHTQSR

SEQ ID NO: 407 - protein, 9 aa, synthetic construct
WRGPGPYAI

SEQ ID NO: 408 - protein, 9 aa, synthetic construct
FEQHAENIE

SEQ ID NO: 409 - protein, 9 aa, synthetic construct
YIGYAGDAQ

SEQ ID NO: 410 - protein, 9 aa, synthetic construct
VPIHDHLRN

SEQ ID NO: 411 - protein, 9 aa, synthetic construct
GWNEEARPP

SEQ ID NO: 412 - protein, 9 aa, synthetic construct
IKYPFARAE

SEQ ID NO: 413 - protein, 9 aa, synthetic construct
GWNSNIGVL

SEQ ID NO: 414 - protein, 9 aa, synthetic construct
AGHYEVHIG

SEQ ID NO: 415 - protein, 9 aa, synthetic construct
DYQHWPYDF

SEQ ID NO: 416 - protein, 7 aa, synthetic construct
FWGDLKT

SEQ ID NO: 417 - protein, 9 aa, synthetic construct
GWNYEHKWP

SEQ ID NO: 418 - protein, 9 aa, synthetic construct
RNFYTFPHE

SEQ ID NO: 419 - protein, 9 aa, synthetic construct
WENFHKLEK

SEQ ID NO: 420 - protein, 9 aa, synthetic construct
NWEGEPSGV

SEQ ID NO: 421 - protein, 9 aa, synthetic construct
RQLLDKPRY

SEQ ID NO: 422 - protein, 9 aa, synthetic construct
KAFANDYYK

SEQ ID NO: 423 - protein, 9 aa, synthetic construct
GHESGQNLW

SEQ ID NO: 424 - protein, 9 aa, synthetic construct
HDFTLKWKF

SEQ ID NO: 425 - protein, 9 aa, synthetic construct
IYASQYDYR

SEQ ID NO: 426 - protein, 9 aa, synthetic construct
KIGYLEFVL

SEQ ID NO: 427 - protein, 9 aa, synthetic construct
NWNDHLSTY

SEQ ID NO: 428 - protein, 10 aa, synthetic construct
FKHRQQLHYT

SEQ ID NO: 429 - protein, 9 aa, synthetic construct
FHRTPGSTT

SEQ ID NO: 430 - protein, 9 aa, synthetic construct
WPWLHSTEQ

SEQ ID NO: 431 - protein, 9 aa, synthetic construct
NNKPPHNDL

SEQ ID NO: 432 - protein, 9 aa, synthetic construct
FLPDKDDPF

SEQ ID NO: 433 - protein, 9 aa, synthetic construct
GRTEVEGSF

SEQ ID NO: 434 - protein, 9 aa, synthetic construct
PEADADDPK

SEQ ID NO: 435 - protein, 9 aa, synthetic construct
RSRYPNYTA

SEQ ID NO: 436 - protein, 9 aa, synthetic construct
NWFHQPWQR

SEQ ID NO: 437 - protein, 9 aa, synthetic construct
WYQPRWKVE

SEQ ID NO: 438 - protein, 9 aa, synthetic construct
FNYNKTEHT

SEQ ID NO: 439 - protein, 9 aa, synthetic construct
GWHNVTLPP

SEQ ID NO: 440 - protein, 9 aa, synthetic construct
FKWDNGISD

SEQ ID NO: 441 - protein, 9 aa, synthetic construct
AVFRQRLGV

SEQ ID NO: 442 - protein, 9 aa, synthetic construct
WYNRYYNDF

SEQ ID NO: 443 - protein, 9 aa, synthetic construct
SNDYYETYL

SEQ ID NO: 444 - protein, 9 aa, synthetic construct
IQWRDHKPR

SEQ ID NO: 445 - protein, 9 aa, synthetic construct
KWYDPTIRP

SEQ ID NO: 446 - protein, 9 aa, synthetic construct
RIGRSTNLW

SEQ ID NO: 447 - protein, 9 aa, synthetic construct
NWDQPDTVL

SEQ ID NO: 448 - protein, 9 aa, synthetic construct
FVENWQKRT

SEQ ID NO: 449 - protein, 9 aa, synthetic construct
FNNHPLSPD

SEQ ID NO: 450 - protein, 9 aa, synthetic construct
NHLAIHELP

SEQ ID NO: 451 - protein, 9 aa, synthetic construct
NWFSDEAPY

SEQ ID NO: 452 - protein, 9 aa, synthetic construct
AGVNRWKWI

SEQ ID NO: 453 - protein, 9 aa, synthetic construct
FTASDTHPA

SEQ ID NO: 454 - protein, 9 aa, synthetic construct
YIHYLKVNE

SEQ ID NO: 455 - protein, 9 aa, synthetic construct
GWNDDHSVG

SEQ ID NO: 456 - protein, 9 aa, synthetic construct
DWDGAAGDA

SEQ ID NO: 457 - protein, 9 aa, synthetic construct
ELNNKDQND

SEQ ID NO: 458 - protein, 9 aa, synthetic construct
FGQPVQGNT

SEQ ID NO: 459 - protein, 9 aa, synthetic construct
YALHINRWP

SEQ ID NO: 460 - protein, 9 aa, synthetic construct
PGEPHHILD

SEQ ID NO: 461 - protein, 9 aa, synthetic construct
WERNASLSE

SEQ ID NO: 462 - protein, 9 aa, synthetic construct
FFWKGNGYR

SEQ ID NO: 463 - protein, 9 aa, synthetic construct
HLKLWKIPN

SEQ ID NO: 464 - protein, 9 aa, synthetic construct
RINNSGLSR

SEQ ID NO: 465 - protein, 9 aa, synthetic construct
PVKYKEILL

SEQ ID NO: 466 - protein, 9 aa, synthetic construct
FLKILAEDD

SEQ ID NO: 467 - protein, 9 aa, synthetic construct
RIGYLEFEI

SEQ ID NO: 468 - protein, 9 aa, synthetic construct
HYYQDVLYR

SEQ ID NO: 469 - protein, 9 aa, synthetic construct
FHGIWWARV

SEQ ID NO: 470 - protein, 9 aa, synthetic construct
YAFITPVTH

SEQ ID NO: 471 - protein, 9 aa, synthetic construct
WLNNFGKNF

SEQ ID NO: 472 - protein, 9 aa, synthetic construct
WNHLYHYPF

SEQ ID NO: 473 - protein, 9 aa, synthetic construct
LFWQKQFYR

SEQ ID NO: 474 - protein, 9 aa, synthetic construct
EQPIEGDLW

SEQ ID NO: 475 - protein, 9 aa, synthetic construct
SWNAPFPAG

SEQ ID NO: 476 - protein, 9 aa, synthetic construct
FVNKGGSDD

SEQ ID NO: 477 - protein, 9 aa, synthetic construct
DEVDEIPHF

SEQ ID NO: 478 - protein, 9 aa, synthetic construct
GWQQVNDVK

SEQ ID NO: 479 - protein, 9 aa, synthetic construct
INVPVTELL

SEQ ID NO: 480 - protein, 9 aa, synthetic construct
VPSDWSKTI

SEQ ID NO: 481 - protein, 9 aa, synthetic construct
GHELYALYA

SEQ ID NO: 482 - protein, 9 aa, synthetic construct
IKEPFIKNV

SEQ ID NO: 483 - protein, 9 aa, synthetic construct
DEPATPTYL

SEQ ID NO: 484 - protein, 9 aa, synthetic construct
WNTLLEFGG

SEQ ID NO: 485 - protein, 9 aa, synthetic construct
KWTNRANKP

SEQ ID NO: 486 - protein, 9 aa, synthetic construct
SNQLIPQPI

SEQ ID NO: 487 - protein, 9 aa, synthetic construct
FKFEIYKHT

SEQ ID NO: 488 - protein, 9 aa, synthetic construct
NWNRPYNDL

SEQ ID NO: 489 - protein, 9 aa, synthetic construct
NWEGNDLAT

SEQ ID NO: 490 - protein, 9 aa, synthetic construct
NPINGQETW

SEQ ID NO: 491 - protein, 9 aa, synthetic construct
IGNGQYWYR

SEQ ID NO: 492 - protein, 9 aa, synthetic construct
DGQGALSLP

SEQ ID NO: 493 - protein, 9 aa, synthetic construct
GKYKTNWYF

SEQ ID NO: 494 - protein, 9 aa, synthetic construct
AWDNTAKHG

SEQ ID NO: 495 - protein, 9 aa, synthetic construct
SQDYNGGQA

SEQ ID NO: 496 - protein, 9 aa, synthetic construct
IYASQYDYR

SEQ ID NO: 497 - protein, 9 aa, synthetic construct
SHQPYRATI

SEQ ID NO: 498 - protein, 9 aa, synthetic construct
GWNLHEVAL

SEQ ID NO: 499 - protein, 9 aa, synthetic construct
GWDLEYSQK

SEQ ID NO: 500 - protein, 9 aa, synthetic construct
NLEHPTDET

SEQ ID NO: 501 - protein, 9 aa, synthetic construct
RLKSNVNIA

SEQ ID NO: 502 - protein, 9 aa, synthetic construct
HWDEYEYDE

SEQ ID NO: 503 - protein, 9 aa, synthetic construct
RWWPWDAVG

SEQ ID NO: 504 - protein, 9 aa, synthetic construct
YNLHHPHKD

SEQ ID NO: 505 - protein, 9 aa, synthetic construct
PHLDYIWNW

SEQ ID NO: 506 - protein, 9 aa, synthetic construct
DLLLRKEEV

SEQ ID NO: 507 - protein, 9 aa, synthetic construct
NWEPAQHPV

SEQ ID NO: 508 - protein, 9 aa, synthetic construct
RWSKDGATA

SEQ ID NO: 509 - protein, 9 aa, synthetic construct
WYYNDNQSG

SEQ ID NO: 510 - protein, 9 aa, synthetic construct
IPLNWQLQE

SEQ ID NO: 511 - protein, 9 aa, synthetic construct
FHGPLDNYT

SEQ ID NO: 512 - protein, 9 aa, synthetic construct
DFDHNFIAD

SEQ ID NO: 513 - protein, 9 aa, synthetic construct
RSGPAHVRR

SEQ ID NO: 514 - protein, 111 aa, synthetic construct
MIPGGLSEAKPATPEIQEIVDKVKPQLEEKTGETYGKLEAVQYKTQVVVYHVRWNYLGTNYYIKVRAGDNKYMHLKVFKSLSLEEPPVETEDLVLTGYQVDKNKDDELTGF

SEQ ID NO: 515 - protein, 111 aa, synthetic construct
MIPGGLSEAKPATPEIQEIVDKVKPQLEEKTGETYGKLEAVQYKTQVVASFIDIGWHGTNYYIKVRAGDNKYMHLKVFKSLREGKHGRNKEDLVLTGYQVDKNKDDELTGF

SEQ ID NO: 516 - protein, 111 aa, synthetic construct
MIPGGLSEAKPATPEIQEIVDKVKPQLEEKTGETYGKLEAVQYKTQVVPNQHFIVPYGTNYYIKVRAGDNKYMHLKVFKSLHWNGGYHGHEDLVLTGYQVDKNKDDELTGF

SEQ ID NO: 517 - protein, 111 aa, synthetic construct
MIPGGLSEAKPATPEIQEIVDKVKPQLEEKTGETYGKLEAVQYKTQVVFQTWDHAWTGTNYYIKVRAGDNKYMHLKVFKSLFAAETQQYYEDLVLTGYQVDKNKDDELTGF

SEQ ID NO: 518 - protein, 110 aa, synthetic construct
MIPGGLSEAKPATPEIQEIVDKVKPQLEEKTGETYGKLEAVQYKTQVVFLDWKQHWRGTNYYIKVRAGDNKYMHLKVFKSLFNDKHNSTEDLVLTGYQVDKNKDDELTGF

SEQ ID NO: 519 - protein, 111 aa, synthetic construct
MIPGGLSEAKPATPEIQEIVDKVKPQLEEKTGETYGKLEAVQYKTQVVLSEAVYIWAGTNYYIKVRAGDNKYMHLKVFKSLNSRWYGPLGEDLVLTGYQVDKNKDDELTGF

SEQ ID NO: 520 - protein, 111 aa, synthetic construct
MIPGGLSEAKPATPEIQEIVDKVKPQLEEKTGETYGKLEAVQYKTQVVRQYYEWSWVGTNYYIKVRAGDNKYMHLKVFKSLFESPTEDNDEDLVLTGYQVDKNKDDELTGF

SEQ ID NO: 521 - protein, 111 aa, synthetic construct
MIPGGLSEAKPATPEIQEIVDKVKPQLEEKTGETYGKLEAVQYKTQVVPLPDPIDWVGTNYYIKVRAGDNKYMHLKVFKSLHIHLNQIYREDLVLTGYQVDKNKDDELTGF

SEQ ID NO: 522 - protein, 111 aa, synthetic construct
MIPGGLSEAKPATPEIQEIVDKVKPQLEEKTGETYGKLEAVQYKTQVVQSHVEGEWLGTNYYIKVRAGDNKYMHLKVFKSLFFAIHRLGIEDLVLTGYQVDKNKDDELTGF

SEQ ID NO: 523 - protein, 111 aa, synthetic construct
MIPGGLSEAKPATPEIQEIVDKVKPQLEEKTGETYGKLEAVQYKTQVVDDVKNGFVQGTNYYIKVRAGDNKYMHLKVFKSLIDRHVYWLEEDLVLTGYQVDKNKDDELTGF

SEQ ID NO: 524 - protein, 111 aa, synthetic construct
MIPGGLSEAKPATPEIQEIVDKVKPQLEEKTGETYGKLEAVQYKTQVVEVQQQENFQGTNYYIKVRAGDNKYMHLKVFKSLHIYEHIVYREDLVLTGYQVDKNKDDELTGF

SEQ ID NO: 525 - protein, 111 aa, synthetic construct
MIPGGLSEAKPATPEIQEIVDKVKPQLEEKTGETYGKLEAVQYKTQVVQLKFPPYVSGTNYYIKVRAGDNKYMHLKVFKSLGWDDKPEWDEDLVLTGYQVDKNKDDELTGF

SEQ ID NO: 526 - protein, 111 aa, synthetic construct
MIPGGLSEAKPATPEIQEIVDKVKPQLEEKTGETYGKLEAVQYKTQVVLFREEKYNYGTNYYIKVRAGDNKYMHLKVFKSLAWGEVVSNAEDLVLTGYQVDKNKDDELTGF

SEQ ID NO: 527 - protein, 111 aa, synthetic construct
MIPGGLSEAKPATPEIQEIVDKVKPQLEEKTGETYGKLEAVQYKTQVVSFLVDPLHLGTNYYIKVRAGDNKYMHLKVFKSLRSFGGEWGWEDLVLTGYQVDKNKDDELTGF

SEQ ID NO: 528 - protein, 111 aa, synthetic construct
MIPGGLSEAKPATPEIQEIVDKVKPQLEEKTGETYGKLEAVQYKTQVVPYFWFQHAKGTNYYIKVRAGDNKYMHLKVFKSLWQSLLDKNEEDLVLTGYQVDKNKDDELTGF

SEQ ID NO: 529 - protein, 111 aa, synthetic construct
MIPGGLSEAKPATPEIQEIVDKVKPQLEEKTGETYGKLEAVQYKTQVVHIQWPPWVEGTNYYIKVRAGDNKYMHLKVFKSLGWTSDNSNFEDLVLTGYQVDKNKDDELTGF

SEQ ID NO: 530 - protein, 111 aa, synthetic construct
MIPGGLSEAKPATPEIQEIVDKVKPQLEEKTGETYGKLEAVQYKTQVVIVYTIWNYVGTNYYIKVRAGDNKYMHLKVFKSLTPQTETEHEEDLVLTGYQVDKNKDDELTGF

SEQ ID NO: 531 - protein, 117 aa, synthetic construct
MIPGGLSEAKPATPEIQEIVDKVKPQLEEKTGETYGKLEAVQYKTQVVEAHQDLHFWGTNYYIKVRAGDNKYMHLKVFKSLLTEHNKKHQERHLDHEDLVLTGYQVDKNKDDELTGF

SEQ ID NO: 532 - protein, 111 aa, synthetic construct
MIPGGLSEAKPATPEIQEIVDKVKPQLEEKTGETYGKLEAVQYKTQVVYRFGPPYVKGTNYYIKVRAGDNKYMHLKVFKSLGWNAFSSPAEDLVLTGYQVDKNKDDELTGF

SEQ ID NO: 533 - protein, 111 aa, synthetic construct
MIPGGLSEAKPATPEIQEIVDKVKPQLEEKTGETYGKLEAVQYKTQVVANSKWHYAFGTNYYIKVRAGDNKYMHLKVFKSLDNAIYRGPTEDLVLTGYQVDKNKDDELTGF

SEQ ID NO: 534 - protein, 111 aa, synthetic construct
MIPGGLSEAKPATPEIQEIVDKVKPQLEEKTGETYGKLEAVQYKTQVVKRPHFLQFSGTNYYIKVRAGDNKYMHLKVFKSLNIGPAKKQLEDLVLTGYQVDKNKDDELTGF

SEQ ID NO: 535 - protein, 111 aa, synthetic construct
MIPGGLSEAKPATPEIQEIVDKVKPQLEEKTGETYGKLEAVQYKTQVVHRLHFPIYYGTNYYIKVRAGDNKYMHLKVFKSLHFTLPTQHPEDLVLTGYQVDKNKDDELTGF

SEQ ID NO: 536 - protein, 111 aa, synthetic construct
MIPGGLSEAKPATPEIQEIVDKVKPQLEEKTGETYGKLEAVQYKTQVVYWEHHHYGFGTNYYIKVRAGDNKYMHLKVFKSLAWPEIDPPVEDLVLTGYQVDKNKDDELTGF

SEQ ID NO: 537 - protein, 111 aa, synthetic construct
MIPGGLSEAKPATPEIQEIVDKVKPQLEEKTGETYGKLEAVQYKTQVVEEGTPEWAPGTNYYIKVRAGDNKYMHLKVFKSLWSDVLYNDFEDLVLTGYQVDKNKDDELTGF

SEQ ID NO: 538 - protein, 111 aa, synthetic construct
MIPGGLSEAKPATPEIQEIVDKVKPQLEEKTGETYGKLEAVQYKTQVVVRHPRWFFIGTNYYIKVRAGDNKYMHLKVFKSLKKHGDEVTLEDLVLTGYQVDKNKDDELTGF

SEQ ID NO: 539 - protein, 111 aa, synthetic construct
MIPGGLSEAKPATPEIQEIVDKVKPQLEEKTGETYGKLEAVQYKTQVVFVFLEVKWDGTNYYIKVRAGDNKYMHLKVFKSLRKERHQFDIEDLVLTGYQVDKNKDDELTGF

SEQ ID NO: 540 - protein, 111 aa, synthetic construct
MIPGGLSEAKPATPEIQEIVDKVKPQLEEKTGETYGKLEAVQYKTQVVDVKPKWQWTGTNYYIKVRAGDNKYMHLKVFKSLFEGEDRDAVEDLVLTGYQVDKNKDDELTGF

SEQ ID NO: 541 - protein, 111 aa, synthetic construct
MIPGGLSEAKPATPEIQEIVDKVKPQLEEKTGETYGKLEAVQYKTQVVAAFSVHPSSGTNYYIKVRAGDNKYMHLKVFKSLEKYHNIWYVEDLVLTGYQVDKNKDDELTGF

SEQ ID NO: 542 - protein, 111 aa, synthetic construct
MIPGGLSEAKPATPEIQEIVDKVKPQLEEKTGETYGKLEAVQYKTQVVFTFVDIHWYGTNYYIKVRAGDNKYMHLKVFKSLRKPKDRQYTEDLVLTGYQVDKNKDDELTGF

SEQ ID NO: 543 - protein, 111 aa, synthetic construct
MIPGGLSEAKPATPEIQEIVDKVKPQLEEKTGETYGKLEAVQYKTQVVKYVVQASKVGTNYYIKVRAGDNKYMHLKVFKSLYGQKDGQKEEDLVLTGYQVDKNKDDELTGF

SEQ ID NO: 544 - protein, 111 aa, synthetic construct
MIPGGLSEAKPATPEIQEIVDKVKPQLEEKTGETYGKLEAVQYKTQVVAQDLSAFQIGTNYYIKVRAGDNKYMHLKVFKSLIFAPLKPWREDLVLTGYQVDKNKDDELTGF

SEQ ID NO: 545 - protein, 111 aa, synthetic construct; identical to SEQ ID NO: 543

SEQ ID NO: 546 - protein, 111 aa, synthetic construct
MIPGGLSEAKPATPEIQEIVDKVKPQLEEKTGETYGKLEAVQYKTQVVNQPQPPWIRGTNYYIKVRAGDNKYMHLKVFKSLNWNSPNLTWEDLVLTGYQVDKNKDDELTGF

SEQ ID NO: 547 - protein, 111 aa, synthetic construct
MIPGGLSEAKPATPEIQEIVDKVKPQLEEKTGETYGKLEAVQYKTQVVTHLNNNAWTGTNYYIKVRAGDNKYMHLKVFKSLFFHNGKIYREDLVLTGYQVDKNKDDELTGF

SEQ ID NO: 548 - protein, 111 aa, synthetic construct
MIPGGLSEAKPATPEIQEIVDKVKPQLEEKTGETYGKLEAVQYKTQVVKYETHFYYEGTNYYIKVRAGDNKYMHLKVFKSLPKRWAYNAYEDLVLTGYQVDKNKDDELTGF

SEQ ID NO: 549 - protein, 111 aa, synthetic construct
MIPGGLSEAKPATPEIQEIVDKVKPQLEEKTGETYGKLEAVQYKTQVVWSFAQVGWHGTNYYIKVRAGDNKYMHLKVFKSLRRFEHLPFYEDLVLTGYQVDKNKDDELTGF

SEQ ID NO: 550 - protein, 111 aa, synthetic construct
MIPGGLSEAKPATPEIQEIVDKVKPQLEEKTGETYGKLEAVQYKTQVVHHFAEVKWYGTNYYIKVRAGDNKYMHLKVFKSLKITGSTLTFEDLVLTGYQVDKNKDDELTGF

SEQ ID NO: 551 - protein, 111 aa, synthetic construct
MIPGGLSEAKPATPEIQEIVDKVKPQLEEKTGETYGKLEAVQYKTQVVRVPLDSNKWGTNYYIKVRAGDNKYMHLKVFKSLFRVRDYSFLEDLVLTGYQVDKNKDDELTGF

SEQ ID NO: 552 - protein, 111 aa, synthetic construct
MIPGGLSEAKPATPEIQEIVDKVKPQLEEKTGETYGKLEAVQYKTQVVEIRPVWDWSGTNYYIKVRAGDNKYMHLKVFKSLFDHESKPTAEDLVLTGYQVDKNKDDELTGF

SEQ ID NO: 553 - protein, 111 aa, synthetic construct
MIPGGLSEAKPATPEIQEIVDKVKPQLEEKTGETYGKLEAVQYKTQVVKRRHFPQWQGTNYYIKVRAGDNKYMHLKVFKSLGDHLNQYPNEDLVLTGYQVDKNKDDELTGF

SEQ ID NO: 554 - protein, 111 aa, synthetic construct
MIPGGLSEAKPATPEIQEIVDKVKPQLEEKTGETYGKLEAVQYKTQVVGTRDEWHWVGTNYYIKVRAGDNKYMHLKVFKSLFVKDDGRVDEDLVLTGYQVDKNKDDELTGF

SEQ ID NO: 555 - protein, 111 aa, synthetic construct
MIPGGLSEAKPATPEIQEIVDKVKPQLEEKTGETYGKLEAVQYKTQVVIQHIWWDFVGTNYYIKVRAGDNKYMHLKVFKSLSRLEPAGHQEDLVLTGYQVDKNKDDELTGF

SEQ ID NO: 556 - protein, 111 aa, synthetic construct
MIPGGLSEAKPATPEIQEIVDKVKPQLEEKTGETYGKLEAVQYKTQVVVQHPQWHWKGTNYYIKVRAGDNKYMHLKVFKSLVQQRDVWINEDLVLTGYQVDKNKDDELTGF

SEQ ID NO: 557 - protein, 111 aa, synthetic construct
MIPGGLSEAKPATPEIQEIVDKVKPQLEEKTGETYGKLEAVQYKTQVVRTKETKKSWGTNYYIKVRAGDNKYMHLKVFKSLQKIHAIFYVEDLVLTGYQVDKNKDDELTGF

SEQ ID NO: 558 - protein, 111 aa, synthetic construct
MIPGGLSEAKPATPEIQEIVDKVKPQLEEKTGETYGKLEAVQYKTQVVREYPHFQGYGTNYYIKVRAGDNKYMHLKVFKSLILNAGGIRWEDLVLTGYQVDKNKDDELTGF

SEQ ID NO: 559 - protein, 111 aa, synthetic construct
MIPGGLSEAKPATPEIQEIVDKVKPQLEEKTGETYGKLEAVQYKTQVVVQHSLWYFQGTNYYIKVRAGDNKYMHLKVFKSLFKHPEENPVEDLVLTGYQVDKNKDDELTGF

SEQ ID NO: 560 - protein, 111 aa, synthetic construct
MIPGGLSEAKPATPEIQEIVDKVKPQLEEKTGETYGKLEAVQYKTQVVGVKQTWNWNGTNYYIKVRAGDNKYMHLKVFKSLFGLNDDEPVEDLVLTGYQVDKNKDDELTGF

SEQ ID NO: 561 - protein, 111 aa, synthetic construct
MIPGGLSEAKPATPEIQEIVDKVKPQLEEKTGETYGKLEAVQYKTQVVTRKHFPQYWGTNYYIKVRAGDNKYMHLKVFKSLNEPLPVTNIEDLVLTGYQVDKNKDDELTGF

SEQ ID NO: 562 - protein, 111 aa, synthetic construct
MIPGGLSEAKPATPEIQEIVDKVKPQLEEKTGETYGKLEAVQYKTQVVKYYAIFDYKGTNYYIKVRAGDNKYMHLKVFKSLFKEKVRNSIEDLVLTGYQVDKNKDDELTGF

SEQ ID NO: 563 - protein, 111 aa, synthetic construct
MIPGGLSEAKPATPEIQEIVDKVKPQLEEKTGETYGKLEAVQYKTQVVGRQVKWAWTGTNYYIKVRAGDNKYMHLKVFKSLFKQHHNISEEDLVLTGYQVDKNKDDELTGF

SEQ ID NO: 564 - protein, 111 aa, synthetic construct
MIPGGLSEAKPATPEIQEIVDKVKPQLEEKTGETYGKLEAVQYKTQVVSHHASPSHIGTNYYIKVRAGDNKYMHLKVFKSLTTDPNWDVHEDLVLTGYQVDKNKDDELTGF

SEQ ID NO: 565 - protein, 111 aa, synthetic construct
MIPGGLSEAKPATPEIQEIVDKVKPQLEEKTGETYGKLEAVQYKTQVVGIREDWNWSGTNYYIKVRAGDNKYMHLKVFKSLFEPKKLEPAEDLVLTGYQVDKNKDDELTGF

SEQ ID NO: 566 - protein, 111 aa, synthetic construct
MIPGGLSEAKPATPEIQEIVDKVKPQLEEKTGETYGKLEAVQYKTQVVAWDWLPAYRGTNYYIKVRAGDNKYMHLKVFKSLFKFQNESFAEDLVLTGYQVDKNKDDELTGF

SEQ ID NO: 567 - protein, 111 aa, synthetic construct
MIPGGLSEAKPATPEIQEIVDKVKPQLEEKTGETYGKLEAVQYKTQVVINQQQIKHSGTNYYIKVRAGDNKYMHLKVFKSLWQHIPPDFWEDLVLTGYQVDKNKDDELTGF

SEQ ID NO: 568 - protein, 111 aa, synthetic construct
MIPGGLSEAKPATPEIQEIVDKVKPQLEEKTGETYGKLEAVQYKTQVVAGWSLPPHLGTNYYIKVRAGDNKYMHLKVFKSLINHAGQKSDEDLVLTGYQVDKNKDDELTGF

SEQ ID NO: 569 - protein, 111 aa, synthetic construct
MIPGGLSEAKPATPEIQEIVDKVKPQLEEKTGETYGKLEAVQYKTQVVGGRHRWNWSGTNYYIKVRAGDNKYMHLKVFKSLFEPSGLAAEEDLVLTGYQVDKNKDDELTGF

SEQ ID NO: 570 - protein, 111 aa, synthetic construct
MIPGGLSEAKPATPEIQEIVDKVKPQLEEKTGETYGKLEAVQYKTQVVELDGDHTWLGTNYYIKVRAGDNKYMHLKVFKSLRQLHRIWYFEDLVLTGYQVDKNKDDELTGF

SEQ ID NO: 571 - protein, 111 aa, synthetic construct
MIPGGLSEAKPATPEIQEIVDKVKPQLEEKTGETYGKLEAVQYKTQVVKQLIIWHWTGTNYYIKVRAGDNKYMHLKVFKSLFKNSNSHLEEDLVLTGYQVDKNKDDELTGF

SEQ ID NO: 572 - protein, 111 aa, synthetic construct
MIPGGLSEAKPATPEIQEIVDKVKPQLEEKTGETYGKLEAVQYKTQVVREPALWHFRGTNYYIKVRAGDNKYMHLKVFKSLTFKVDTWKVEDLVLTGYQVDKNKDDELTGF

SEQ ID NO: 573 - protein, 111 aa, synthetic construct
MIPGGLSEAKPATPEIQEIVDKVKPQLEEKTGETYGKLEAVQYKTQVVPQVQEPWWIGTNYYIKVRAGDNKYMHLKVFKSLYKAYIQGWNEDLVLTGYQVDKNKDDELTGF

SEQ ID NO: 574 - protein, 111 aa, synthetic construct
MIPGGLSEAKPATPEIQEIVDKVKPQLEEKTGETYGKLEAVQYKTQVVPTGTHQWAAGTNYYIKVRAGDNKYMHLKVFKSLWASLESGQNEDLVLTGYQVDKNKDDELTGF

SEQ ID NO: 575 - protein, 111 aa, synthetic construct
MIPGGLSEAKPATPEIQEIVDKVKPQLEEKTGETYGKLEAVQYKTQVVFRQHFPNFSGTNYYIKVRAGDNKYMHLKVFKSLNGLEDPTQDEDLVLTGYQVDKNKDDELTGF

SEQ ID NO: 576 - protein, 111 aa, synthetic construct
MIPGGLSEAKPATPEIQEIVDKVKPQLEEKTGETYGKLEAVQYKTQVVQEYEDQEWAGTNYYIKVRAGDNKYMHLKVFKSLHLWNQQIYREDLVLTGYQVDKNKDDELTGF

SEQ ID NO: 577 - protein, 111 aa, synthetic construct
MIPGGLSEAKPATPEIQEIVDKVKPQLEEKTGETYGKLEAVQYKTQVVIAHIHWKYFGTNYYIKVRAGDNKYMHLKVFKSLHLAKGQWREEDLVLTGYQVDKNKDDELTGF

SEQ ID NO: 578 - protein, 111 aa, synthetic construct
MIPGGLSEAKPATPEIQEIVDKVKPQLEEKTGETYGKLEAVQYKTQVVDYRAIWHWWGTNYYIKVRAGDNKYMHLKVFKSLFHAEGDIWDEDLVLTGYQVDKNKDDELTGF

SEQ ID NO: 579 - protein, 111 aa, synthetic construct
MIPGGLSEAKPATPEIQEIVDKVKPQLEEKTGETYGKLEAVQYKTQVVNLKPDVQGSGTNYYIKVRAGDNKYMHLKVFKSLIVVKRYWYVEDLVLTGYQVDKNKDDELTGF

SEQ ID NO: 580 - protein, 111 aa, synthetic construct
MIPGGLSEAKPATPEIQEIVDKVKPQLEEKTGETYGKLEAVQYKTQVVSTKNLWRWSGTNYYIKVRAGDNKYMHLKVFKSLFNGAGGPDSEDLVLTGYQVDKNKDDELTGF

SEQ ID NO: 581 - protein, 111 aa, synthetic construct
MIPGGLSEAKPATPEIQEIVDKVKPQLEEKTGETYGKLEAVQYKTQVVRVQGLWNFQGTNYYIKVRAGDNKYMHLKVFKSLGHHGDHEKWEDLVLTGYQVDKNKDDELTGF

SEQ ID NO: 582 - protein, 111 aa, synthetic construct
MIPGGLSEAKPATPEIQEIVDKVKPQLEEKTGETYGKLEAVQYKTQVVYKVDNNNYQGTNYYIKVRAGDNKYMHLKVFKSLITWQLNQPREDLVLTGYQVDKNKDDELTGF

SEQ ID NO: 583 - protein, 111 aa, synthetic construct
MIPGGLSEAKPATPEIQEIVDKVKPQLEEKTGETYGKLEAVQYKTQVVIEEPSAFWWGTNYYIKVRAGDNKYMHLKVFKSLLRVRHSAYREDLVLTGYQVDKNKDDELTGF

SEQ ID NO: 584 - protein, 111 aa, synthetic construct
MIPGGLSEAKPATPEIQEIVDKVKPQLEEKTGETYGKLEAVQYKTQVVASFPEISWYGTNYYIKVRAGDNKYMHLKVFKSLKNYPKTGVTEDLVLTGYQVDKNKDDELTGF

SEQ ID NO: 585 - protein, 111 aa, synthetic construct
MIPGGLSEAKPATPEIQEIVDKVKPQLEEKTGETYGKLEAVQYKTQVVIHEPEIWWYGTNYYIKVRAGDNKYMHLKVFKSLHKADRQKEREDLVLTGYQVDKNKDDELTGF

SEQ ID NO: 586 - protein, 111 aa, synthetic construct
MIPGGLSEAKPATPEIQEIVDKVKPQLEEKTGETYGKLEAVQYKTQVVDWGWGIPWVGTNYYIKVRAGDNKYMHLKVFKSLWLYGVKRLPEDLVLTGYQVDKNKDDELTGF

SEQ ID NO: 587 - protein, 111 aa, synthetic construct
MIPGGLSEAKPATPEIQEIVDKVKPQLEEKTGETYGKLEAVQYKTQVVRRWELWSFQGTNYYIKVRAGDNKYMHLKVFKSLWQEHTDSVEEDLVLTGYQVDKNKDDELTGF

SEQ ID NO: 588 - protein, 111 aa, synthetic construct
MIPGGLSEAKPATPEIQEIVDKVKPQLEEKTGETYGKLEAVQYKTQVVINQHENFKQGTNYYIKVRAGDNKYMHLKVFKSLHLFHGLWYIEDLVLTGYQVDKNKDDELTGF

SEQ ID NO: 589 - protein, 111 aa, synthetic construct
MIPGGLSEAKPATPEIQEIVDKVKPQLEEKTGETYGKLEAVQYKTQVVHDFTIRYPWGTNYYIKVRAGDNKYMHLKVFKSLEYHVTKRDQEDLVLTGYQVDKNKDDELTGF

SEQ ID NO: 590 - protein, 111 aa, synthetic construct
MIPGGLSEAKPATPEIQEIVDKVKPQLEEKTGETYGKLEAVQYKTQVVWDRPRWGWTGTNYYIKVRAGDNKYMHLKVFKSLFKESSRNDEEDLVLTGYQVDKNKDDELTGF

SEQ ID NO: 591 - protein, 111 aa, synthetic construct
MIPGGLSEAKPATPEIQEIVDKVKPQLEEKTGETYGKLEAVQYKTQVVDQGWWENWAGTNYYIKVRAGDNKYMHLKVFKSLIWENNLRIHEDLVLTGYQVDKNKDDELTGF

SEQ ID NO: 592 - protein, 111 aa, synthetic construct
MIPGGLSEAKPATPEIQEIVDKVKPQLEEKTGETYGKLEAVQYKTQVVIWHSEWSYTGTNYYIKVRAGDNKYMHLKVFKSLFTRHSEPWEEDLVLTGYQVDKNKDDELTGF

SEQ ID NO: 593 - protein, 111 aa, synthetic construct
MIPGGLSEAKPATPEIQEIVDKVKPQLEEKTGETYGKLEAVQYKTQVVRRKHFPQWPGTNYYIKVRAGDNKYMHLKVFKSLDLQPREVFQEDLVLTGYQVDKNKDDELTGF

SEQ ID NO: 594 - protein, 111 aa, synthetic construct
MIPGGLSEAKPATPEIQEIVDKVKPQLEEKTGETYGKLEAVQYKTQVVKAYVIWSYKGTNYYIKVRAGDNKYMHLKVFKSLFYGEPRGKGEDLVLTGYQVDKNKDDELTGF

SEQ ID NO: 595 - protein, 111 aa, synthetic construct
MIPGGLSEAKPATPEIQEIVDKVKPQLEEKTGETYGKLEAVQYKTQVVFWQLHRYGFGTNYYIKVRAGDNKYMHLKVFKSLEWNRLSPLWEDLVLTGYQVDKNKDDELTGF

SEQ ID NO: 596 - protein, 111 aa, synthetic construct
MIPGGLSEAKPATPEIQEIVDKVKPQLEEKTGETYGKLEAVQYKTQVVIYHELWNFYGTNYYIKVRAGDNKYMHLKVFKSLDNNGDGNWQEDLVLTGYQVDKNKDDELTGF

SEQ ID NO: 597 - protein, 111 aa, synthetic construct
MIPGGLSEAKPATPEIQEIVDKVKPQLEEKTGETYGKLEAVQYKTQVVLIRHLWSWSGTNYYIKVRAGDNKYMHLKVFKSLFEGENHQIDEDLVLTGYQVDKNKDDELTGF

SEQ ID NO: 598 - protein, 111 aa, synthetic construct
MIPGGLSEAKPATPEIQEIVDKVKPQLEEKTGETYGKLEAVQYKTQVVNDGKSLVLAGTNYYIKVRAGDNKYMHLKVFKSLRLWDDRVSGEDLVLTGYQVDKNKDDELTGF

SEQ ID NO: 599 - protein, 111 aa, synthetic construct
MIPGGLSEAKPATPEIQEIVDKVKPQLEEKTGETYGKLEAVQYKTQVVDVRGQWAWTGTNYYIKVRAGDNKYMHLKVFKSLFQPRTKATEEDLVLTGYQVDKNKDDELTGF

SEQ ID NO: 600 - protein, 111 aa, synthetic construct
MIPGGLSEAKPATPEIQEIVDKVKPQLEEKTGETYGKLEAVQYKTQVVKRLHLPQYPGTNYYIKVRAGDNKYMHLKVFKSLGNIEPITDLEDLVLTGYQVDKNKDDELTGF

SEQ ID NO: 601 - protein, 111 aa, synthetic construct
MIPGGLSEAKPATPEIQEIVDKVKPQLEEKTGETYGKLEAVQYKTQVVAVHAPQYAPGTNYYIKVRAGDNKYMHLKVFKSLWSEKVAVFPEDLVLTGYQVDKNKDDELTGF

SEQ ID NO: 602 - protein, 111 aa, synthetic construct
MIPGGLSEAKPATPEIQEIVDKVKPQLEEKTGETYGKLEAVQYKTQVVDFRVAWHWVGTNYYIKVRAGDNKYMHLKVFKSLFNHRPGKPDEDLVLTGYQVDKNKDDELTGF

SEQ ID NO: 603 - protein, 111 aa, synthetic construct
MIPGGLSEAKPATPEIQEIVDKVKPQLEEKTGETYGKLEAVQYKTQVVLGWWFGINQGTNYYIKVRAGDNKYMHLKVFKSLAHLQYEWGGEDLVLTGYQVDKNKDDELTGF

SEQ ID NO: 604 - protein, 111 aa, synthetic construct
MIPGGLSEAKPATPEIQEIVDKVKPQLEEKTGETYGKLEAVQYKTQVVNQEHFRRFIGTNYYIKVRAGDNKYMHLKVFKSLNPWLHPQDFEDLVLTGYQVDKNKDDELTGF

SEQ ID NO: 605 - protein, 109 aa, synthetic construct
MIPGGLSEAKPATPEIQEIVDKVKPQLEEKTGETYGKLEAVQYKTQVDIDDDLWSGTNYYIKVRAGDNKYMHLKVFKSLILWPTKPEREDLVLTGYQVDKNKDDELTGF

SEQ ID NO: 606 - protein, 111 aa, synthetic construct
MIPGGLSEAKPATPEIQEIVDKVKPQLEEKTGETYGKLEAVQYKTQVDQWGVDLVYTGTNYYIKVRAGDNKYMHLKVFKSLIVKRAVILWEDLVLTGYQVDKNKDDELTGF

SEQ ID NO: 607 - protein, 111 aa, synthetic construct
MIPGGLSEAKPATPEIQEIVDKVKPQLEEKTGETYGKLEAVQYKTQVDKVYLVWAWTGTNYYIKVRAGDNKYMHLKVFKSLFQKGSNKPEEDLVLTGYQVDKNKDDELTGF

SEQ ID NO: 608 - protein, 111 aa, synthetic construct
MIPGGLSEAKPATPEIQEIVDKVKPQLEEKTGETYGKLEAVQYKTQVDTIHPQAKYWGTNYYIKVRAGDNKYMHLKVFKSLWKNLLKPIHEDLVLTGYQVDKNKDDELTGF

SEQ ID NO: 609 - protein, 110 aa, synthetic construct
MIPGGLSEAKPATPEIQEIVDKVKPQLEEKTGETYGKLEAVQYKTQVDFNQSRHGHGTNYYIKVRAGDNKYMHLKVFKSLELLHGLWWVEDLVLTGYQVDKNKDDELTGF

SEQ ID NO: 610 - protein, 111 aa, synthetic construct
MIPGGLSEAKPATPEIQEIVDKVKPQLEEKTGETYGKLEAVQYKTQVDELDDAVKYWGTNYYIKVRAGDNKYMHLKVFKSLWWKTISADFEDLVLTGYQVDKNKDDELTGF

SEQ ID NO: 611 - protein, 111 aa, synthetic construct
MIPGGLSEAKPATPEIQEIVDKVKPQLEEKTGETYGKLEAVQYKTQVDWAIWRDKKVGTNYYIKVRAGDNKYMHLKVFKSLPIFEIVREGEDLVLTGYQVDKNKDDELTGF

SEQ ID NO: 612 - protein, 111 aa, synthetic construct
MIPGGLSEAKPATPEIQEIVDKVKPQLEEKTGETYGKLEAVQYKTQVDTWGERLHYPGTNYYIKVRAGDNKYMHLKVFKSLWHKPKDNIIEDLVLTGYQVDKNKDDELTGF

SEQ ID NO: 613 - protein, 111 aa, synthetic construct
MIPGGLSEAKPATPEIQEIVDKVKPQLEEKTGETYGKLEAVQYKTQVDHTDVDWHFWGTNYYIKVRAGDNKYMHLKVFKSLAPAAPPWEFEDLVLTGYQVDKNKDDELTGF

SEQ ID NO: 614 - protein, 111 aa, synthetic construct
MIPGGLSEAKPATPEIQEIVDKVKPQLEEKTGETYGKLEAVQYKTQVDHHHRFAKWWGTNYYIKVRAGDNKYMHLKVFKSLWFNENREDTEDLVLTGYQVDKNKDDELTGF

SEQ ID NO: 615 - protein, 111 aa, synthetic construct
MIPGGLSEAKPATPEIQEIVDKVKPQLEEKTGETYGKLEAVQYKTQVDRKAAVVARHGTNYYIKVRAGDNKYMHLKVFKSLWPFLVIEKYEDLVLTGYQVDKNKDDELTGF

SEQ ID NO: 616 - protein, 111 aa, synthetic construct
MIPGGLSEAKPATPEIQEIVDKVKPQLEEKTGETYGKLEAVQYKTQVDFSFPLVHWYGTNYYIKVRAGDNKYMHLKVFKSLRFRDGGHHVEDLVLTGYQVDKNKDDELTGF

SEQ ID NO: 617 - protein, 111 aa, synthetic construct
MIPGGLSEAKPATPEIQEIVDKVKPQLEEKTGETYGKLEAVQYKTQVDNAHPLVKNWGTNYYIKVRAGDNKYMHLKVFKSLWRNDTSSIYEDLVLTGYQVDKNKDDELTGF

SEQ ID NO: 618 - protein, 111 aa, synthetic construct
MIPGGLSEAKPATPEIQEIVDKVKPQLEEKTGETYGKLEAVQYKTQVDETSENVIQWGTNYYIKVRAGDNKYMHLKVFKSLWPADILTQHEDLVLTGYQVDKNKDDELTGF

SEQ ID NO: 619 - protein, 111 aa, synthetic construct
MIPGGLSEAKPATPEIQEIVDKVKPQLEEKTGETYGKLEAVQYKTQVDFSDLEWEGQGTNYYIKVRAGDNKYMHLKVFKSLEKKWGWIYIEDLVLTGYQVDKNKDDELTGF

SEQ ID NO: 620 - protein, 111 aa, synthetic construct
MIPGGLSEAKPATPEIQEIVDKVKPQLEEKTGETYGKLEAVQYKTQVDFTDIHWSTWGTNYYIKVRAGDNKYMHLKVFKSLIPAWQQIHVEDLVLTGYQVDKNKDDELTGF

SEQ ID NO: 621 - protein, 111 aa, synthetic construct
MIPGGLSEAKPATPEIQEIVDKVKPQLEEKTGETYGKLEAVQYKTQVDREHASVKFWGTNYYIKVRAGDNKYMHLKVFKSLWLEATPHTQEDLVLTGYQVDKNKDDELTGF

SEQ ID NO: 622 - protein, 111 aa, synthetic construct
MIPGGLSEAKPATPEIQEIVDKVKPQLEEKTGETYGKLEAVQYKTQVDISGVGLVPNGTNYYIKVRAGDNKYMHLKVFKSLNWNYPQQPFEDLVLTGYQVDKNKDDELTGF

SEQ ID NO: 623 - protein, 111 aa, synthetic construct
MIPGGLSEAKPATPEIQEIVDKVKPQLEEKTGETYGKLEAVQYKTQVDHTQPKALDWGTNYYIKVRAGDNKYMHLKVFKSLWGSILTDGPEDLVLTGYQVDKNKDDELTGF

SEQ ID NO: 624 - protein, 111 aa, synthetic construct
MIPGGLSEAKPATPEIQEIVDKVKPQLEEKTGETYGKLEAVQYKTQVDIQDRLGGFIGTNYYIKVRAGDNKYMHLKVFKSLHFYRGILYREDLVLTGYQVDKNKDDELTGF

SEQ ID NO: 625 - protein, 111 aa, synthetic construct
MIPGGLSEAKPATPEIQEIVDKVKPQLEEKTGETYGKLEAVQYKTQVDRFYHQWHFLGTNYYIKVRAGDNKYMHLKVFKSLDLRAPRNDNEDLVLTGYQVDKNKDDELTGF

SEQ ID NO: 626 - protein, 110 aa, synthetic construct
MIPGGLSEAKPATPEIQEIVDKVKPQLEEKTGETYGKLEAVQYKTQVDWVTRHHTLVGTNYYIKVRAGDNKYMHLKVFKSLPPFKHWHLEDLVLTGYQVDKNKDDELTGF

SEQ ID NO: 627 - protein, 111 aa, synthetic construct
MIPGGLSEAKPATPEIQEIVDKVKPQLEEKTGETYGKLEAVQYKTQVDQSSDLAKLWGTNYYIKVRAGDNKYMHLKVFKSLWNLNSDKSSEDLVLTGYQVDKNKDDELTGF

SEQ ID NO: 628 - protein, 111 aa, synthetic construct; identical to SEQ ID NO: 625

SEQ ID NO: 629 - protein, 110 aa, synthetic construct; identical to SEQ ID NO: 626

SEQ ID NO: 630 - protein, 111 aa, synthetic construct
MIPGGLSEAKPATPEIQEIVDKVKPQLEEKTGETYGKLEAVQYKTQVDHEVRKTYEFGTNYYIKVRAGDNKYMHLKVFKSLWFGGWPLAQEDLVLTGYQVDKNKDDELTGF

SEQ ID NO: 631 - protein, 111 aa, synthetic construct
MIPGGLSEAKPATPEIQEIVDKVKPQLEEKTGETYGKLEAVQYKTQVDVISIPLVWNGTNYYIKVRAGDNKYMHLKVFKSLIFQEYYPHREDLVLTGYQVDKNKDDELTGF

SEQ ID NO: 632 - protein, 111 aa, synthetic construct
MIPGGLSEAKPATPEIQEIVDKVKPQLEEKTGETYGKLEAVQYKTQVDSWVFKPFYHGTNYYIKVRAGDNKYMHLKVFKSLSGPEFFSRHEDLVLTGYQVDKNKDDELTGF

SEQ ID NO: 633 - protein, 111 aa, synthetic construct
MIPGGLSEAKPATPEIQEIVDKVKPQLEEKTGETYGKLEAVQYKTQVDPEVSITRWQGTNYYIKVRAGDNKYMHLKVFKSLYETHGNIITEDLVLTGYQVDKNKDDELTGF

SEQ ID NO: 634 - protein, 111 aa, synthetic construct
MIPGGLSEAKPATPEIQEIVDKVKPQLEEKTGETYGKLEAVQYKTQVDGNFAHERWQGTNYYIKVRAGDNKYMHLKVFKSLYIARDFDWTEDLVLTGYQVDKNKDDELTGF

SEQ ID NO: 635 - protein, 111 aa, synthetic construct
MIPGGLSEAKPATPEIQEIVDKVKPQLEEKTGETYGKLEAVQYKTQVDVIHIPHQWHGTNYYIKVRAGDNKYMHLKVFKSLIFHEPKPPREDLVLTGYQVDKNKDDELTGF

SEQ ID NO: 636 - protein, 111 aa, synthetic construct
MIPGGLSEAKPATPEIQEIVDKVKPQLEEKTGETYGKLEAVQYKTQVDNLVYYQRDWGTNYYIKVRAGDNKYMHLKVFKSLEPGRWRWPKEDLVLTGYQVDKNKDDELTGF

SEQ ID NO: 637 - protein, 111 aa, synthetic construct
MIPGGLSEAKPATPEIQEIVDKVKPQLEEKTGETYGKLEAVQYKTQVDQVLWEIFEHGTNYYIKVRAGDNKYMHLKVFKSLFHQWSPALHEDLVLTGYQVDKNKDDELTGF

SEQ ID NO: 638 - protein, 111 aa, synthetic construct
MIPGGLSEAKPATPEIQEIVDKVKPQLEEKTGETYGKLEAVQYKTQVVTRLHFPQYQGTNYYIKVRAGDNKYMHLKVFKSLEIEVNPYANEDLVLTGYQVDKNKDDELTGF

SEQ ID NO: 639 - protein, 111 aa, synthetic construct
MIPGGLSEAKPATPEIQEIVDKVKPQLEEKTGETYGKLEAVQYKTQVVYEPHHPYPWGTNYYIKVRAGDNKYMHLKVFKSLQYHPPYERFEDLVLTGYQVDKNKDDELTGF

SEQ ID NO: 640 - protein, 111 aa, synthetic construct
MIPGGLSEAKPATPEIQEIVDKVKPQLEEKTGETYGKLEAVQYKTQVVTQAIPWREWGTNYYIKVRAGDNKYMHLKVFKSLIPEWPKDASEDLVLTGYQVDKNKDDELTGF

SEQ ID NO: 641 - protein, 111 aa, synthetic construct
MIPGGLSEAKPATPEIQEIVDKVKPQLEEKTGETYGKLEAVQYKTQVVPEVDKAEWWGTNYYIKVRAGDNKYMHLKVFKSLIRYGLRHEREDLVLTGYQVDKNKDDELTGF

SEQ ID NO: 642 - protein, 111 aa, synthetic construct
MIPGGLSEAKPATPEIQEIVDKVKPQLEEKTGETYGKLEAVQYKTQVVQRVHFPDWPGTNYYIKVRAGDNKYMHLKVFKSLNKENQDEEDEDLVLTGYQVDKNKDDELTGF

SEQ ID NO: 643 - protein, 111 aa, synthetic construct
MIPGGLSEAKPATPEIQEIVDKVKPQLEEKTGETYGKLEAVQYKTQVVPQPEWNEWGVTNYYIKVRAGDNKYMHLKVFKSLIFWNEKLIREDLVLTGYQVDKNKDDELTGF

SEQ ID NO: 644 - protein, 111 aa, synthetic construct
MIPGGLSEAKPATPEIQEIVDKVKPQLEEKTGETYGKLEAVQYKTQVVNQWEWPVDLGTNYYIKVRAGDNKYMHLKVFKSLKVYRTHHIFEDLVLTGYQVDKNKDDELTGF

SEQ ID NO: 645 - protein, 111 aa, synthetic construct
MIPGGLSEAKPATPEIQEIVDKVKPQLEEKTGETYGKLEAVQYKTQVVERYHFPQFTGTNYYIKVRAGDNKYMHLKVFKSLNHDQVRWNLEDLVLTGYQVDKNKDDELTGF

SEQ ID NO: 646 - protein, 111 aa, synthetic construct
MIPGGLSEAKPATPEIQEIVDKVKPQLEEKTGETYGKLEAVQYKTQVVLYSESRDYKGTNYYIKVRAGDNKYMHLKVFKSLHAYKGSIYREDLVLTGYQVDKNKDDELTGF

SEQ ID NO: 647 - protein, 111 aa, synthetic construct
MIPGGLSEAKPATPEIQEIVDKVKPQLEEKTGETYGKLEAVQYKTQVVFWFEDIQWLGTNYYIKVRAGDNKYMHLKVFKSLRFREDVDIREDLVLTGYQVDKNKDDELTGF

SEQ ID NO: 648 - protein, 110 aa, synthetic construct
MIPGGLSEAKPATPEIQEIVDKVKPQLEEKTGETYGKLEAVQYKTQVVAVYPTFKYGTNYYIKVRAGDNKYMHLKVFKSLKVEYLDFITEDLVLTGYQVDKNKDDELTGF

SEQ ID NO: 649 - protein, 110 aa, synthetic construct
MIPGGLSEAKPATPEIQEIVDKVKPQLEEKTGETYGKLEAVQYKTQVVVLRWQWGYLGTNYYIKVRAGDNKYMHLKVFKSLQLLYEDNDEDLVLTGYQVDKNKDDELTGF

SEQ ID NO: 650 - protein, 111 aa, synthetic construct
MIPGGLSEAKPATPEIQEIVDKVKPQLEEKTGETYGKLEAVQYKTQVVLWPLQPKNWGTNYYIKVRAGDNKYMHLKVFKSLGWNYLEAIAEDLVLTGYQVDKNKDDELTGF

SEQ ID NO: 651 - protein, 111 aa, synthetic construct
MIPGGLSEAKPATPEIQEIVDKVKPQLEEKTGETYGKLEAVQYKTQVVDNVRHGWLSGTNYYIKVRAGDNKYMHLKVFKSLFALLWSAYGEDLVLTGYQVDKNKDDELTGF

SEQ ID NO: 652 - protein, 111 aa, synthetic construct
MIPGGLSEAKPATPEIQEIVDKVKPQLEEKTGETYGKLEAVQYKTQVVGLIQPPYAQGTNYYIKVRAGDNKYMHLKVFKSLGWQYIDLFHEDLVLTGYQVDKNKDDELTGF

SEQ ID NO: 653 - protein, 111 aa, synthetic construct
MIPGGLSEAKPATPEIQEIVDKVKPQLEEKTGETYGKLEAVQYKTQVVRQVGVWAYEGTNYYIKVRAGDNKYMHLKVFKSLGRTVEGGRWEDLVLTGYQVDKNKDDELTGF

SEQ ID NO: 654 - protein, 111 aa, synthetic construct
MIPGGLSEAKPATPEIQEIVDKVKPQLEEKTGETYGKLEAVQYKTQVVDLHTLKSGVGTNYYIKVRAGDNKYMHLKVFKSLYIKHHLWWIEDLVLTGYQVDKNKDDELTGF

SEQ ID NO: 655 - protein, 111 aa, synthetic construct
MIPGGLSEAKPATPEIQEIVDKVKPQLEEKTGETYGKLEAVQYKTQVVSWYKLWSYSGTNYYIKVRAGDNKYMHLKVFKSLFEGRHKDPEEDLVLTGYQVDKNKDDELTGF

SEQ ID NO: 656 - protein, 111 aa, synthetic construct
MIPGGLSEAKPATPEIQEIVDKVKPQLEEKTGETYGKLEAVQYKTQVVDWPSHAKFWGTNYYIKVRAGDNKYMHLKVFKSLFLPDKDDPFEDLVLTGYQVDKNKDDELTGF

SEQ ID NO: 657 - protein, 111 aa, synthetic construct
MIPGGLSEAKPATPEIQEIVDKVKPQLEEKTGETYGKLEAVQYKTQVVAYQSPPYHFGTNYYIKVRAGDNKYMHLKVFKSLRQIAGYIAIEDLVLTGYQVDKNKDDELTGF

SEQ ID NO: 658 - protein, 111 aa, synthetic construct
MIPGGLSEAKPATPEIQEIVDKVKPQLEEKTGETYGKLEAVQYKTQVVYKNVGPWVNGTNYYIKVRAGDNKYMHLKVFKSLYRTNRGHEREDLVLTGYQVDKNKDDELTGF

SEQ ID NO: 659 - protein, 111 aa, synthetic construct
MIPGGLSEAKPATPEIQEIVDKVKPQLEEKTGETYGKLEAVQYKTQVVSKRSAWHWTGTNYYIKVRAGDNKYMHLKVFKSLFWNAPDDRQEDLVLTGYQVDKNKDDELTGF

SEQ ID NO: 660 - protein, 110 aa, synthetic construct
MIPGGLSEAKPATPEIQEIVDKVKPQLEEKTGETYGKLEAVQYKTQVVVHITVEWEGTNYYIKVRAGDNKYMHLKVFKSLIYQRHTQSREDLVLTGYQVDKNKDDELTGF

SEQ ID NO: 661 - protein, 111 aa, synthetic construct
MIPGGLSEAKPATPEIQEIVDKVKPQLEEKTGETYGKLEAVQYKTQVVRDYDPFPYTGTNYYIKVRAGDNKYMHLKVFKSLWRGPGPYAIEDLVLTGYQVDKNKDDELTGF

SEQ ID NO: 662 - protein, 111 aa, synthetic construct
MIPGGLSEAKPATPEIQEIVDKVKPQLEEKTGETYGKLEAVQYKTQVVKTLGVWHWSGTNYYIKVRAGDNKYMHLKVFKSLFEQHAENIEEDLVLTGYQVDKNKDDELTGF

SEQ ID NO: 663 - protein, 111 aa, synthetic construct
MIPGGLSEAKPATPEIQEIVDKVKPQLEEKTGETYGKLEAVQYKTQVVREVGLWRFQGTNYYIKVRAGDNKYMHLKVFKSLYIGYAGDAQEDLVLTGYQVDKNKDDELTGF

SEQ ID NO: 664 - protein, 111 aa, synthetic construct
MIPGGLSEAKPATPEIQEIVDKVKPQLEEKTGETYGKLEAVQYKTQVVWEEVNWRNWGTNYYIKVRAGDNKYMHLKVFKSLVPIHDHLRNEDLVLTGYQVDKNKDDELTGF

SEQ ID NO: 665 - protein, 111 aa, synthetic construct
MIPGGLSEAKPATPEIQEIVDKVKPQLEEKTGETYGKLEAVQYKTQVVRAQLRPWVAGTNYYIKVRAGDNKYMHLKVFKSLGWNEEARPPEDLVLTGYQVDKNKDDELTGF

SEQ ID NO: 666 - protein, 111 aa, synthetic construct
MIPGGLSEAKPATPEIQEIVDKVKPQLEEKTGETYGKLEAVQYKTQVVYPWSWNGHYGTNYYIKVRAGDNKYMHLKVFKSLIKYPFARAEEDLVLTGYQVDKNKDDELTGF

SEQ ID NO: 667 - protein, 111 aa, synthetic construct
MIPGGLSEAKPATPEIQEIVDKVKPQLEEKTGETYGKLEAVQYKTQVVPQIGAPYVKGTNYYIKVRAGDNKYMHLKVFKSLGWNSNIGVLEDLVLTGYQVDKNKDDELTGF

SEQ ID NO: 668 - protein, 111 aa, synthetic construct
MIPGGLSEAKPATPEIQEIVDKVKPQLEEKTGETYGKLEAVQYKTQVVWRTGQKWQVGTNYYIKVRAGDNKYMHLKVFKSLAGHYEVHIGEDLVLTGYQVDKNKDDELTGF

SEQ ID NO: 669 - protein, 111 aa, synthetic construct
MIPGGLSEAKPATPEIQEIVDKVKPQLEEKTGETYGKLEAVQYKTQVVIQQLYKQTWGTNYYIKVRAGDNKYMHLKVFKSLDYQHWPYDFEDLVLTGYQVDKNKDDELTGF

SEQ ID NO: 670 - protein, 109 aa, synthetic construct
MIPGGLSEAKPATPEIQEIVDKVKPQLEEKTGETYGKLEAVQYKTQVVIYYPIWDFHGTNYYIKVRAGDNKYMHLKVFKSLFWGDLKTEDLVLTGYQVDKNKDDELTGF

SEQ ID NO: 671 - protein, 111 aa, synthetic construct
MIPGGLSEAKPATPEIQEIVDKVKPQLEEKTGETYGKLEAVQYKTQVVDNVANIVPHGTNYYIKVRAGDNKYMHLKVFKSLGWNYEHKWPEDLVLTGYQVDKNKDDELTGF

SEQ ID NO: 672 - protein, 111 aa, synthetic construct
MIPGGLSEAKPATPEIQEIVDKVKPQLEEKTGETYGKLEAVQYKTQVVHVHWAWNYLGTNYYIKVRAGDNKYMHLKVFKSLRNFYTFPHEEDLVLTGYQVDKNKDDELTGF

SEQ ID NO: 673 - protein, 111 aa, synthetic construct
MIPGGLSEAKPATPEIQEIVDKVKPQLEEKTGETYGKLEAVQYKTQVVGPWYFGSAWGTNYYIKVRAGDNKYMHLKVFKSLWENFHKLEKEDLVLTGYQVDKNKDDELTGF

SEQ ID NO: 674 - protein, 111 aa, synthetic construct
MIPGGLSEAKPATPEIQEIVDKVKPQLEEKTGETYGKLEAVQYKTQVVVLERPPYVKGTNYYIKVRAGDNKYMHLKVFKSLNWEGEPSGVEDLVLTGYQVDKNKDDELTGF

SEQ ID NO: 675 - protein, 111 aa, synthetic construct
MIPGGLSEAKPATPEIQEIVDKVKPQLEEKTGETYGKLEAVQYKTQVVFTFSSIGWWGTNYYIKVRAGDNKYMHLKVFKSLRQLLDKPRYEDLVLTGYQVDKNKDDELTGF

SEQ ID NO: 676 - protein, 111 aa, synthetic construct
MIPGGLSEAKPATPEIQEIVDKVKPQLEEKTGETYGKLEAVQYKTQVVISASEIRWYGTNYYIKVRAGDNKYMHLKVFKSLKAFANDYYKEDLVLTGYQVDKNKDDELTGF

SEQ ID NO: 677 - protein, 111 aa, synthetic construct
MIPGGLSEAKPATPEIQEIVDKVKPQLEEKTGETYGKLEAVQYKTQVVRIWGFWAFQGTNYYIKVRAGDNKYMHLKVFKSLGHESGQNLWEDLVLTGYQVDKNKDDELTGF

SEQ ID NO: 678 - protein, 111 aa, synthetic construct
MIPGGLSEAKPATPEIQEIVDKVKPQLEEKTGETYGKLEAVQYKTQVVNEAESSRFLGTNYYIKVRAGDNKYMHLKVFKSLHDFTLKWKFEDLVLTGYQVDKNKDDELTGF

SEQ ID NO: 679 - protein, 111 aa, synthetic construct
MIPGGLSEAKPATPEIQEIVDKVKPQLEEKTGETYGKLEAVQYKTQVVVAGLKWHWEGTNYYIKVRAGDNKYMHLKVFKSLIYASQYDYREDLVLTGYQVDKNKDDELTGF

SEQ ID NO: 680 - protein, 111 aa, synthetic construct
MIPGGLSEAKPATPEIQEIVDKVKPQLEEKTGETYGKLEAVQYKTQVVYRNKQWYNEGTNYYIKVRAGDNKYMHLKVFKSLKIGYLEFVLEDLVLTGYQVDKNKDDELTGF

SEQ ID NO: 681 - protein, 111 aa, synthetic construct
MIPGGLSEAKPATPEIQEIVDKVKPQLEEKTGETYGKLEAVQYKTQVVWFDTWPVPEGTNYYIKVRAGDNKYMHLKVFKSLNWNDHLSTYEDLVLTGYQVDKNKDDELTGF

SEQ ID NO: 682 - protein, 112 aa, synthetic construct
MIPGGLSEAKPATPEIQEIVDKVKPQLEEKTGETYGKLEAVQYKTQVVYANIGPWLYGTNYYIKVRAGDNKYMHLKVFKSLFKHRQQLHYTEDLVLTGYQVDKNKDDELTGF

SEQ ID NO: 683 - protein, 111 aa, synthetic construct
MIPGGLSEAKPATPEIQEIVDKVKPQLEEKTGETYGKLEAVQYKTQVVHQFQEIKWFGTNYYIKVRAGDNKYMHLKVFKSLFHRTPGSTTEDLVLTGYQVDKNKDDELTGF

SEQ ID NO: 684 - protein, 111 aa, synthetic construct
MIPGGLSEAKPATPEIQEIVDKVKPQLEEKTGETYGKLEAVQYKTQVVIDHQNHPTEGTNYYIKVRAGDNKYMHLKVFKSLWPWLHSTEQEDLVLTGYQVDKNKDDELTGF

SEQ ID NO: 685 - protein, 111 aa, synthetic construct
MIPGGLSEAKPATPEIQEIVDKVKPQLEEKTGETYGKLEAVQYKTQVVSVFQYTYKFGTNYYIKVRAGDNKYMHLKVFKSLNNKPPHNDLEDLVLTGYQVDKNKDDELTGF

SEQ ID NO: 686 - protein, 111 aa, synthetic construct; identical to SEQ ID NO: 656

SEQ ID NO: 687 - protein, 111 aa, synthetic construct
MIPGGLSEAKPATPEIQEIVDKVKPQLEEKTGETYGKLEAVQYKTQVVRQVTIWHFEGTNYYIKVRAGDNKYMHLKVFKSLGRTEVEGSFEDLVLTGYQVDKNKDDELTGF

SEQ ID NO: 688 - protein, 111 aa, synthetic construct
MIPGGLSEAKPATPEIQEIVDKVKPQLEEKTGETYGKLEAVQYKTQVVWRRWFPQWPGTNYYIKVRAGDNKYMHLKVFKSLPEADADDPKEDLVLTGYQVDKNKDDELTGF

SEQ ID NO: 689 - protein, 111 aa, synthetic construct
MIPGGLSEAKPATPEIQEIVDKVKPQLEEKTGETYGKLEAVQYKTQVVFWYPEIGWLGTNYYIKVRAGDNKYMHLKVFKSLRSRYPNYTAEDLVLTGYQVDKNKDDELTGF

SEQ ID NO: 690 - protein, 111 aa, synthetic construct
MIPGGLSEAKPATPEIQEIVDKVKPQLEEKTGETYGKLEAVQYKTQVVISEDSWWPHGTNYYIKVRAGDNKYMHLKVFKSLNWFHQPWQREDLVLTGYQVDKNKDDELTGF

SEQ ID NO: 691 - protein, 111 aa, synthetic construct
MIPGGLSEAKPATPEIQEIVDKVKPQLEEKTGETYGKLEAVQYKTQVVDHLPHAKYWGTNYYIKVRAGDNKYMHLKVFKSLWYQPRWKVEEDLVLTGYQVDKNKDDELTGF

SEQ ID NO: 692 - protein, 111 aa, synthetic construct
MIPGGLSEAKPATPEIQEIVDKVKPQLEEKTGETYGKLEAVQYKTQVVFKTDDARWIGTNYYIKVRAGDNKYMHLKVFKSLFNYNKTEHTEDLVLTGYQVDKNKDDELTGF

SEQ ID NO: 693 - protein, 111 aa, synthetic construct
MIPGGLSEAKPATPEIQEIVDKVKPQLEEKTGETYGKLEAVQYKTQVVLQGSRLPIEGTNYYIKVRAGDNKYMHLKVFKSLGWHNVTLPPEDLVLTGYQVDKNKDDELTGF

SEQ ID NO: 694 - protein, 111 aa, synthetic construct
MIPGGLSEAKPATPEIQEIVDKVKPQLEEKTGETYGKLEAVQYKTQVVKAYKVFNYIGTNYYIKVRAGDNKYMHLKVFKSLFKWDNGISDEDLVLTGYQVDKNKDDELTGF

SEQ ID NO: 695 - protein, 111 aa, synthetic construct
MIPGGLSEAKPATPEIQEIVDKVKPQLEEKTGETYGKLEAVQYKTQVVLKTNPSPSDGTNYYIKVRAGDNKYMHLKVFKSLAVFRQRLGVEDLVLTGYQVDKNKDDELTGF

SEQ ID NO: 696 - protein, 111 aa, synthetic construct
MIPGGLSEAKPATPEIQEIVDKVKPQLEEKTGETYGKLEAVQYKTQVVRQTDSVKWWGTNYYIKVRAGDNKYMHLKVFKSLWYNRYYNDFEDLVLTGYQVDKNKDDELTGF

SEQ ID NO: 697 - protein, 111 aa, synthetic construct
MIPGGLSEAKPATPEIQEIVDKVKPQLEEKTGETYGKLEAVQYKTQVVHRKHFHQFTGTNYYIKVRAGDNKYMHLKVFKSLSNDYYETYLEDLVLTGYQVDKNKDDELTGF

SEQ ID NO: 698 - protein, 111 aa, synthetic construct
MIPGGLSEAKPATPEIQEIVDKVKPQLEEKTGETYGKLEAVQYKTQVVVVLFASGWEGTNYYIKVRAGDNKYMHLKVFKSLIQWRDHKPREDLVLTGYQVDKNKDDELTGF

SEQ ID NO: 699 - protein, 111 aa, synthetic construct
MIPGGLSEAKPATPEIQEIVDKVKPQLEEKTGETYGKLEAVQYKTQVVYDNVKEYPWGTNYYIKVRAGDNKYMHLKVFKSLKWYDPTIRPEDLVLTGYQVDKNKDDELTGF

SEQ ID NO: 700 - protein, 111 aa, synthetic construct
MIPGGLSEAKPATPEIQEIVDKVKPQLEEKTGETYGKLEAVQYKTQVVIQDWTVGWEGTNYYIKVRAGDNKYMHLKVFKSLRIGRSTNLWEDLVLTGYQVDKNKDDELTGF

SEQ ID NO: 701 - protein, 111 aa, synthetic construct
MIPGGLSEAKPATPEIQEIVDKVKPQLEEKTGETYGKLEAVQYKTQVVPQENAPWVLGTNYYIKVRAGDNKYMHLKVFKSLNWDQPDTVLEDLVLTGYQVDKNKDDELTGF

SEQ ID NO: 702 - protein, 111 aa, synthetic construct
MIPGGLSEAKPATPEIQEIVDKVKPQLEEKTGETYGKLEAVQYKTQVVFQSTDSFWHGTNYYIKVRAGDNKYMHLKVFKSLFVENWQKRTEDLVLTGYQVDKNKDDELTGF

SEQ ID NO: 703 - protein, 111 aa, synthetic construct
MIPGGLSEAKPATPEIQEIVDKVKPQLEEKTGETYGKLEAVQYKTQVVGVKSTFHWSGTNYYIKVRAGDNKYMHLKVFKSLFNNHPLSPDEDLVLTGYQVDKNKDDELTGF

SEQ ID NO: 704 - protein, 111 aa, synthetic construct
MIPGGLSEAKPATPEIQEIVDKVKPQLEEKTGETYGKLEAVQYKTQVVLRYHFPEFHGTNYYIKVRAGDNKYMHLKVFKSLNHLAIHELPEDLVLTGYQVDKNKDDELTGF

SEQ ID NO: 705 - protein, 111 aa, synthetic construct
MIPGGLSEAKPATPEIQEIVDKVKPQLEEKTGETYGKLEAVQYKTQVVTENNSWWPIGTNYYIKVRAGDNKYMHLKVFKSLNWFSDEAPYEDLVLTGYQVDKNKDDELTGF

SEQ ID NO: 706 - protein, 111 aa, synthetic construct
MIPGGLSEAKPATPEIQEIVDKVKPQLEEKTGETYGKLEAVQYKTQVVLRHPVQDDDGTNYYIKVRAGDNKYMHLKVFKSLAGVNRWKWIEDLVLTGYQVDKNKDDELTGF

SEQ ID NO: 707 - protein, 111 aa, synthetic construct
MIPGGLSEAKPATPEIQEIVDKVKPQLEEKTGETYGKLEAVQYKTQVVEERRIWNWTGTNYYIKVRAGDNKYMHLKVFKSLFTASDTHPAEDLVLTGYQVDKNKDDELTGF

SEQ ID NO: 708 - protein, 111 aa, synthetic construct
MIPGGLSEAKPATPEIQEIVDKVKPQLEEKTGETYGKLEAVQYKTQVVVGVGRDFPKGTNYYIKVRAGDNKYMHLKVFKSLYIHYLKVNEEDLVLTGYQVDKNKDDELTGF

SEQ ID NO: 709 - protein, 111 aa, synthetic construct
MIPGGLSEAKPATPEIQEIVDKVKPQLEEKTGETYGKLEAVQYKTQVVLLWTPPYVDGTNYYIKVRAGDNKYMHLKVFKSLGWNDDHSVGEDLVLTGYQVDKNKDDELTGF

SEQ ID NO: 710 - protein, 111 aa, synthetic construct
MIPGGLSEAKPATPEIQEIVDKVKPQLEEKTGETYGKLEAVQYKTQVVWKPQDSVNWGTNYYIKVRAGDNKYMHLKVFKSLDWDGAAGDAEDLVLTGYQVDKNKDDELTGF

SEQ ID NO: 711 - protein, 114 aa, synthetic construct
MIPGGLSEAKPATPEIQEIVDKVKPQLEEKTGETYGKLEAVQYKTQVVSSPLHWELWHWSGTNYYIKVRAGDNKYMHLKVFKSLELNNKDQNDEDLVLTGYQVDKNKDDELTGF

SEQ ID NO: 712 - protein, 111 aa, synthetic construct
MIPGGLSEAKPATPEIQEIVDKVKPQLEEKTGETYGKLEAVQYKTQVVRDHELVNQWGTNYYIKVRAGDNKYMHLKVFKSLFGQPVQGNTEDLVLTGYQVDKNKDDELTGF

SEQ ID NO: 713 - protein, 111 aa, synthetic construct
MIPGGLSEAKPATPEIQEIVDKVKPQLEEKTGETYGKLEAVQYKTQVVYGPHHQTYAGTNYYIKVRAGDNKYMHLKVFKSLYALHINRWPEDLVLTGYQVDKNKDDELTGF

SEQ ID NO: 714 - protein, 111 aa, synthetic construct
MIPGGLSEAKPATPEIQEIVDKVKPQLEEKTGETYGKLEAVQYKTQVVQRAHFKQWYGTNYYIKVRAGDNKYMHLKVFKSLPGEPHHILDEDLVLTGYQVDKNKDDELTGF

SEQ ID NO: 715 - protein, 111 aa, synthetic construct
MIPGGLSEAKPATPEIQEIVDKVKPQLEEKTGETYGKLEAVQYKTQVVIRHVIWSYSGTNYYIKVRAGDNKYMHLKVFKSLWERNASLSEEDLVLTGYQVDKNKDDELTGF

SEQ ID NO: 716 - protein, 111 aa, synthetic construct
MIPGGLSEAKPATPEIQEIVDKVKPQLEEKTGETYGKLEAVQYKTQVVYHGPPKEWLGTNYYIKVRAGDNKYMHLKVFKSLFFWKGNGYREDLVLTGYQVDKNKDDELTGF

SEQ ID NO: 717 - protein, 111 aa, synthetic construct
MIPGGLSEAKPATPEIQEIVDKVKPQLEEKTGETYGKLEAVQYKTQVVSLRPHEQLYGTNYYIKVRAGDNKYMHLKVFKSLHLKLWKIPNEDLVLTGYQVDKNKDDELTGF

SEQ ID NO: 718 - protein, 111 aa, synthetic construct
MIPGGLSEAKPATPEIQEIVDKVKPQLEEKTGETYGKLEAVQYKTQVVVFLSEIHWYGTNYYIKVRAGDNKYMHLKVFKSLRINNSGLSREDLVLTGYQVDKNKDDELTGF

SEQ ID NO: 719 - protein, 111 aa, synthetic construct
MIPGGLSEAKPATPEIQEIVDKVKPQLEEKTGETYGKLEAVQYKTQVVRADVNVIWWGTNYYIKVRAGDNKYMHLKVFKSLPVKYKEILLEDLVLTGYQVDKNKDDELTGF

SEQ ID NO: 720 - protein, 111 aa, synthetic construct
MIPGGLSEAKPATPEIQEIVDKVKPQLEEKTGETYGKLEAVQYKTQVVDTKTPWLWTGTNYYIKVRAGDNKYMHLKVFKSLFLKILAEDDEDLVLTGYQVDKNKDDELTGF

SEQ ID NO: 721 - protein, 111 aa, synthetic construct
MIPGGLSEAKPATPEIQEIVDKVKPQLEEKTGETYGKLEAVQYKTQVVQWKKTQYFFGTNYYIKVRAGDNKYMHLKVFKSLRIGYLEFEIEDLVLTGYQVDKNKDDELTGF

SEQ ID NO: 722 - protein, 111 aa, synthetic construct
MIPGGLSEAKPATPEIQEIVDKVKPQLEEKTGETYGKLEAVQYKTQVVIAKEENHWRGTNYYIKVRAGDNKYMHLKVFKSLHYYQDVLYREDLVLTGYQVDKNKDDELTGF

SEQ ID NO: 723 - protein, 111 aa, synthetic construct
MIPGGLSEAKPATPEIQEIVDKVKPQLEEKTGETYGKLEAVQYKTQVVWEYAGDYLEGTNYYIKVRAGDNKYMHLKVFKSLFHGIWWARVEDLVLTGYQVDKNKDDELTGF

SEQ ID NO: 724 - protein, 111 aa, synthetic construct
MIPGGLSEAKPATPEIQEIVDKVKPQLEEKTGETYGKLEAVQYKTQVVTHPEDAYSWGTNYYIKVRAGDNKYMHLKVFKSLLKPGGLLDFEDLVLTGYQVDKNKDDELTGF

SEQ ID NO: 725 - protein, 111 aa, synthetic construct
MIPGGLSEAKPATPEIQEIVDKVKPQLEEKTGETYGKLEAVQYKTQVVYAFITPVTHGTNYYIKVRAGDNKYMHLKVFKSLWLNNFGKNFEDLVLTGYQVDKNKDDELTGF

SEQ ID NO: 726 - protein, 111 aa, synthetic construct
MIPGGLSEAKPATPEIQEIVDKVKPQLEEKTGETYGKLEAVQYKTQVVKQHTKEWAPGTNYYIKVRAGDNKYMHLKVFKSLWNHLYHYPFEDLVLTGYQVDKNKDDELTGF

SEQ ID NO: 727 - protein, 111 aa, synthetic construct
MIPGGLSEAEPATPEIQEIVDKVKPQLEEKTGETYGKLEAVQYKTQVVIKIHHGSWEGTNYYIKVRAGDNKYMHLKVFKSLLFWQKQFYREDLVLTGYQVDKNKDDELTGF

SEQ ID NO: 728 - protein, 111 aa, synthetic construct
MIPGGLSEAKPATPEIQEIVDKVKPQLEEKTGETYGKLEAVQYKTQVVRRVFFPEFPGTNYYIKVRAGDNKYMHLKVFKSLEQPIEGDLWEDLVLTGYQVDKNKDDELTGF

SEQ ID NO: 729 - protein, 111 aa, synthetic construct
MIPGGLSEAKPATPEIQEIVDKVKPQLEEKTGETYGKLEAVQYKTQVVFTEVKPFVEGTNYYIKVRAGDNKYMHLKVFKSLSWNAPFPAGEDLVLTGYQVDKNKDDELTGF

SEQ ID NO: 730 - protein, 111 aa, synthetic construct
MIPGGLSEAKPATPEIQEIVDKVKPQLEEKTGETYGKLEAVQYKTQVVKKYVIFFYEGTNYYIKVRAGDNKYMHLKVFKSLFVNKGGSDDEDLVLTGYQVDKNKDDELTGF

SEQ ID NO: 731 - protein, 111 aa, synthetic construct
MIPGGLSEAKPATPEIQEIVDKVKPQLEEKTGETYGKLEAVQYKTQVVWRVYFPDFKGTNYYIKVRAGDNKYMHLKVFKSLDEVDEIPHFEDLVLTGYQVDKNKDDELTGF

SEQ ID NO: 732 - protein, 111 aa, synthetic construct
MIPGGLSEAKPATPEIQEIVDKVKPQLEEKTGETYGKLEAVQYKTQVVPLTIPPFVEGTNYYIKVRAGDNKYMHLKVFKSLGWQQVNDVKEDLVLTGYQVDKNKDDELTGF

SEQ ID NO: 733 - protein, 111 aa, synthetic construct
MIPGGLSEAKPATPEIQEIVDKVKPQLEEKTGETYGKLEAVQYKTQVVNRWNIREFEGTNYYIKVRAGDNKYMHLKVFKSLINVPVTELLEDLVLTGYQVDKNKDDELTGF

SEQ ID NO: 734 - protein, 111 aa, synthetic construct
MIPGGLSEAKPATPEIQEIVDKVKPQLEEKTGETYGKLEAVQYKTQVVIQDVDWRLWGTNYYIKVRAGDNKYMHLKVFKSLVPSDWSKTIEDLVLTGYQVDKNKDDELTGF

SEQ ID NO: 735 - protein, 111 aa, synthetic construct
MIPGGLSEAEPATPEIQEIVDKVKPQLEEKTGETYGKLEAVQYKTQVVQRDHFPEFPGTNYYIKVRAGDNKYMHLKVFKSLGHELYALYAEDLVLTGYQVDKNKDDELTGF

SEQ ID NO: 736 - protein, 111 aa, synthetic construct
MIPGGLSEAKPATPEIQEIVDKVKPQLEEKTGETYGKLEAVQYKTQVVFPWDQENYYGTNYYIKVRAGDNKYMHLKVFKSLIKEPFIKNVEDLVLTGYQVDKNKDDELTGF

SEQ ID NO: 737 - protein, 111 aa, synthetic construct
MIPGGLSEAKPATPEIQEIVDKVKPQLEEKTGETYGKLEAVQYKTQVVTRRHFVQFTGTNYYIKVRAGDNKYMHLKVFKSLDEPATPTYLEDLVLTGYQVDKNKDDELTGF

SEQ ID NO: 738 - protein, 111 aa, synthetic construct
MIPGGLSEAKPATPEIQEIVDKVKPQLEEKTGETYGKLEAVQYKTQVVDLHTPHYAPGTNYYIKVRAGDNKYMHLKVFKSLWNTLLEFGGEDLVLTGYQVDKNKDDELTGF

SEQ ID NO: 739 - protein, 111 aa, synthetic construct
MIPGGLSEAKPATPEIQEIVDKVKPQLEEKTGETYGKLEAVQYKTQVVYKHDDEYGFGTNYYIKVRAGDNKYMHLKVFKSLKWTNRANKPEDLVLTGYQVDKNKDDELTGF

SEQ ID NO: 740 - protein, 111 aa, synthetic construct
MIPGGLSEAKPATPEIQEIVDKVKPQLEEKTGETYGKLEAVQYKTQVVYVRWHEYGFGTNYYIKVRAGDNKYMHLKVFKSLSNQLIPQPIEDLVLTGYQVDKNKDDELTGF

SEQ ID NO: 741 - protein, 111 aa, synthetic construct
MIPGGLSEAKPATPEIQEIVDKVKPQLEEKTGETYGKLEAVQYKTQVVVWTSDDAWQGTNYYIKVRAGDNKYMHLKVFKSLFKFEIYKHTEDLVLTGYQVDKNKDDELTGF

SEQ ID NO: 742 - protein, 111 aa, synthetic construct
MIPGGLSEAKPATPEIQEIVDKVKPQLEEKTGETYGKLEAVQYKTQVVHSSSKPYIHGTNYYIKVRAGDNKYMHLKVFKSLNWNRPYNDLEDLVLTGYQVDKNKDDELTGF

SEQ ID NO: 743 - protein, 111 aa, synthetic construct
MIPGGLSEAKPATPEIQEIVDKVKPQLEEKTGETYGKLEAVQYKTQVVHVSNFIIPHGTNYYIKVRAGDNKYMHLKVFKSLNWEGNDLATEDLVLTGYQVDKNKDDELTGF

SEQ ID NO: 744 - protein, 111 aa, synthetic construct
MIPGGLSEAKPATPEIQEIVDKVKPQLEEKTGETYGKLEAVQYKTQVVARWHWFHHFGTNYYIKVRAGDNKYMHLKVFKSLNPINGQETWEDLVLTGYQVDKNKDDELTGF

SEQ ID NO: 745 - protein, 111 aa, synthetic construct
MIPGGLSEAKPATPEIQEIVDKVKPQLEEKTGETYGKLEAVQYKTQVVTKHSATFFHGTNYYIKVRAGDNKYMHLKVFKSLIGNGQYWYREDLVLTGYQVDKNKDDELTGF

SEQ ID NO: 746 - protein, 111 aa, synthetic construct
MIPGGLSEAKPATPEIQEIVDKVKPQLEEKTGETYGKLEAVQYKTQVVLRYHFQHFPGTNYYIKVRAGDNKYMHLKVFKSLDGQGALSLPEDLVLTGYQVDKNKDDELTGF

SEQ ID NO: 747 - protein, 111 aa, synthetic construct
MIPGGLSEAKPATPEIQEIVDKVKPQLEEKTGETYGKLEAVQYKTQVVRNSGVWEFEGTNYYIKVRAGDNKYMHLKVFKSLGKYKTNWYFEDLVLTGYQVDKNKDDELTGF

SEQ ID NO: 748 - protein, 111 aa, synthetic construct
MIPGGLSEAKPATPEIQEIVDKVKPQLEEKTGETYGKLEAVQYKTQVVWNPPSLPISGTNYYIKVRAGDNKYMHLKVFKSLAWDNTAKHGEDLVLTGYQVDKNKDDELTGF

SEQ ID NO: 749 - protein, 111 aa, synthetic construct
MIPGGLSEAKPATPEIQEIVDKVKPQLEEKTGETYGKLEAVQYKTQVVRDGTFWDFWGTNYYIKVRAGDNKYMHLKVFKSLSQDYNGGQAEDLVLTGYQVDKNKDDELTGF

SEQ ID NO: 750 - protein, 111 aa, synthetic construct; identical to SEQ ID NO: 679

SEQ ID NO: 751 - protein, 111 aa, synthetic construct
MIPGGLSEAKPATPEIQEIVDKVKPQLEEKTGETYGKLEAVQYKTQVVRHKGLWQFEGTNYYIKVRAGDNKYMHLKVFKSLSHQPYRATIEDLVLTGYQVDKNKDDELTGF

SEQ ID NO: 752 - protein, 111 aa, synthetic construct
MIPGGLSEAKPATPEIQEIVDKVKPQLEEKTGETYGKLEAVQYKTQVVLTIPPTAFSGTNYYIKVRAGDNKYMHLKVFKSLGWNLHEVALEDLVLTGYQVDKNKDDELTGF

SEQ ID NO: 753 - protein, 111 aa, synthetic construct
MIPGGLSEAKPATPEIQEIVDKVKPQLEEKTGETYGKLEAVQYKTQVVLLYQAPFVQGTNYYIKVRAGDNKYMHLKVFKSLGWDLEYSQKEDLVLTGYQVDKNKDDELTGF

SEQ ID NO: 754 - protein, 111 aa, synthetic construct
MIPGGLSEAKPATPEIQEIVDKVKPQLEEKTGETYGKLEAVQYKTQVVSRLHFPNFQGTNYYIKVRAGDNKYMHLKVFKSLNLEHPTDETEDLVLTGYQVDKNKDDELTGF

SEQ ID NO: 755 - protein, 111 aa, synthetic construct
MIPGGLSEAKPATPEIQEIVDKVKPQLEEKTGETYGKLEAVQYKTQVVVEWSESGYDGTNYYIKVRAGDNKYMHLKVFKSLRLKSNVNIAEDLVLTGYQVDKNKDDELTGF

SEQ ID NO: 756 - protein, 111 aa, synthetic construct
MIPGGLSEAKPATPEIQEIVDKVKPQLEEKTGETYGKLEAVQYKTQVVWKQITLPVDGTNYYIKVRAGDNKYMHLKVFKSLHWDEYEYDEEDLVLTGYQVDKNKDDELTGF

SEQ ID NO: 757 - protein, 111 aa, synthetic construct
MIPGGLSEAKPATPEIQEIVDKVKPQLEEKTGETYGKLEAVQYKTQVVWDSEALPIDGTNYYIKVRAGDNKYMHLKVFKSLRWWPWDAVGEDLVLTGYQVDKNKDDELTGF

SEQ ID NO: 758 - protein, 111 aa, synthetic construct
MIPGGLSEAKPATPEIQEIVDKVKPQLEEKTGETYGKLEAVQYKTQVVGYLELWKWSGTNYYIKVRAGDNKYMHLKVFKSLYNLHHPHKDEDLVLTGYQVDKNKDDELTGF

SEQ ID NO: 759 - protein, 111 aa, synthetic construct
MIPGGLSEAKPATPEIQEIVDKVKPQLEEKTGETYGKLEAVQYKTQVVERFHFPQFPGTNYYIKVRAGDNKYMHLKVFKSLPHLDYIWNWEDLVLTGYQVDKNKDDELTGF

SEQ ID NO: 760 - protein, 111 aa, synthetic construct
MIPGGLSEAKPATPEIQEIVDKVKPQLEEKTGETYGKLEAVQYKTQVVRREHFPRWPGTNYYIKVRAGDNKYMHLKVFKSLDLLLRKEEVEDLVLTGYQVDKNKDDELTGF

SEQ ID NO: 761 - protein, 111 aa, synthetic construct
MIPGGLSEAKPATPEIQEIVDKVKPQLEEKTGETYGKLEAVQYKTQVVLQLPPRPHDGTNYYIKVRAGDNKYMHLKVFKSLNWEPAQHPVEDLVLTGYQVDKNKDDELTGF

SEQ ID NO: 762 - protein, 111 aa, synthetic construct
MIPGGLSEAKPATPEIQEIVDKVKPQLEEKTGETYGKLEAVQYKTQVVIYYPIWGFQGTNYYIKVRAGDNKYMHLKVFKSLRWSKDGATAEDLVLTGYQVDKNKDDELTGF

SEQ ID NO: 763 - protein, 111 aa, synthetic construct
MIPGGLSEAKPATPEIQEIVDKVKPQLEEKTGETYGKLEAVQYKTQVVRDPDVFVFSGTNYYIKVRAGDNKYMHLKVFKSLWYYNDNQSGEDLVLTGYQVDKNKDDELTGF

SEQ ID NO: 764 - protein, 111 aa, synthetic construct
MIPGGLSEAKPATPEIQEIVDKVKPQLEEKTGETYGKLEAVQYKTQVVATDQDWRKWGTNYYIKVRAGDNKYMHLKVFKSLIPLNWQLQEEDLVLTGYQVDKNKDDELTGF

SEQ ID NO: 765 - protein, 111 aa, synthetic construct
MIPGGLSEAKPATPEIQEIVDKVKPQLEEKTGETYGKLEAVQYKTQVVAVRPIFKWAGTNYYIKVRAGDNKYMHLKVFKSLFHGPLDNYTEDLVLTGYQVDKNKDDELTGF

SEQ ID NO: 766 - protein, 111 aa, synthetic construct
MIPGGLSEAKPATPEIQEIVDKVKPQLEEKTGETYGKLEAVQYKTQVVTRPHFPGWNGTNYYIKVRAGDNKYMHLKVFKSLDFDHNFIADEDLVLTGYQVDKNKDDELTGF

SEQ ID NO: 767 - protein, 111 aa, synthetic construct
MIPGGLSEAKPATPEIQEIVDKVKPQLEEKTGETYGKLEAVQYKTQVVISDVSVGWEGTNYYIKVRAGDNKYMHLKVFKSLRSGPAHVRREDLVLTGYQVDKNKDDELTGF

SEQ ID NO: 768 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgtttaccatgttcgttggaactacctgggtacgaactactacatcaaggttcgtgcgggtgacaacaagtatatgcacctgaaagtgtttaagagcctgtctctggaagaaccaccagttgaaaccgaagatttggtgctgacgggctaccaggttgacaagaacaaagatgacgagctgacgggtttc

SEQ ID NO: 769 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgcatctttcatcgatatcggttggcatggtacgaactactacatcaaggttcgtgcgggtgacaacaagtatatgcacctgaaagtgtttaagagcctgagagaaggtaaacatggtagaaacaaagaagatttggtgctgacgggctaccaggttgacaagaacaaagatgacgagctgacgggtttc

SEQ ID NO: 770 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcccaaaccagcatttcatcgttccatacggtacgaactactacatcaaggttcgtgcgggtgacaacaagtatatgcacctgaaagtgtttaagagcctgcattggaacggtggttaccatggtcatgaagatttggtgctgacgggctaccaggttgacaagaacaaagatgacgagctgacgggtttc

SEQ ID NO: 771 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcttccagacctgggatcatgcatggaccggtacgaactactacatcaaggttcgtgcgggtgacaacaagtatatgcacctgaaagtgtttaagagcctgttcgcagcagaaacccagcagtactacgaagatttggtgctgacgggctaccaggttgacaagaacaaagatgacgagctgacgggtttc

SEQ ID NO: 772 - DNA, 330 bp, synthetic construct
  source 1..330: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcttcctggattggaaacagcattggcgtggtacgaactactacatcaaggttcgtgcgggtgacaacaagtatatgcacctgaaagtgtttaagagcctgttcaacgataaacataactctaccgaagatttggtgctgacgggctaccaggttgacaagaacaaagatgacgagctgacgggtttc

SEQ ID NO: 773 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgtctgaagcagtttacatctgggcaggtacgaactactacatcaaggttcgtgcgggtgacaacaagtatatgcacctgaaagtgtttaagagcctgaactctagatggtacggtccactgggtgaagatttggtgctgacgggctaccaggttgacaagaacaaagatgacgagctgacgggtttc

SEQ ID NO: 774 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcagtactacgaatggtcttgggttggtacgaactactacatcaaggttcgtgcgggtgacaacaagtatatgcacctgaaagtgtttaagagcctgttcgaatccccaaccgaagataacgatgaagatttggtgctgacgggctaccaggttgacaagaacaaagatgacgagctgacgggtttc

SEQ ID NO: 775 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcccactgccagatccaatcgattgggttggtacgaactactacatcaaggttcgtgcgggtgacaacaagtatatgcacctgaaagtgtttaagagcctgcatatccatctgaaccagatctacagagaagatttggtgctgacgggctaccaggttgacaagaacaaagatgacgagctgacgggtttc

SEQ ID NO: 776 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccagtctcatgttgaaggtgaatggctgggtacgaactactacatcaaggttcgtgcgggtgacaacaagtatatgcacctgaaagtgtttaagagcctgttcttcgcaatccatagactgggtatcgaagatttggtgctgacgggctaccaggttgacaagaacaaagatgacgagctgacgggtttc

SEQ ID NO: 777 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgatgatgttaaaaacggtttcgttcagggtacgaactactacatcaaggttcgtgcgggtgacaacaagtatatgcacctgaaagtgtttaagagcctgatcgatagacatgtttactggctggaagaagatttggtgctgacgggctaccaggttgacaagaacaaagatgacgagctgacgggtttc

SEQ ID NO: 778 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgaagttcagcagcaggaaaacttccagggtacgaactactacatcaaggttcgtgcgggtgacaacaagtatatgcacctgaaagtgtttaagagcctgcatatctacgaacatatcgtttacagagaagatttggtgctgacgggctaccaggttgacaagaacaaagatgacgagctgacgggtttc

SEQ ID NO: 779 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccagctgaaattcccaccatacgtttctggtacgaactactacatcaaggttcgtgcgggtgacaacaagtatatgcacctgaaagtgtttaagagcctgggttgggatgataaaccagaatgggatgaagatttggtgctgacgggctaccaagttgacaagaacaaagatgacgagctgacgggtttc

SEQ ID NO: 780 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgttccgtgaagaaaaatacaactacggtacgaactactacatcaaggttcgtgcgggtgacaacaagtatatgcacctgaaagtgtttaagagcctggcatggggtgaagttgtttctaacgcagaagatttggtgctgacgggctaccaggttgacaagaacaaagatgacgagctgacgggtttc

SEQ ID NO: 781 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctctttcctggttgatccactgcatctgggtacgaactactacatcaaggttcgtgcgggtgacaacaagtatatgcacctgaaagtgtttaagagcctgagatctttcggtggtgaatggggttgggaagatttggtgctgacgggctaccaggttgacaagaacaaagatgacgagctgacgggtttc

SEQ ID NO: 782 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcccatacttctggttccagcatgcaaaaggtacgaactactacatcaaggttcgtgcgggtgacaacaagtatatgcacctgaaagtgtttaagagcctgtggcagtctctgctggataaaaacgaagaagatttggtgctgacgggctaccaggttgacaagaacaaagatgacgagctgacgggtttc

SEQ ID NO: 783 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccatatccagtggccaccatgggttgaaggtacgaactactacatcaaggttcgtgcgggtgacaacaagtatatgcacctgaaagtgtttaagagcctgggttggacctctgataactctaacttcgaagatttggtgctgacgggctaccaggttgacaagaacaaagatgacgagctgacgggtttc

SEQ ID NO: 784 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatcgtttacaccatctggaactacgttggtacgaactactacatcaaggttcgtgcgggtgacaacaagtatatgcacctgaaagtgtttaagagcctgaccccacagaccgaaaccgaacatgaagaagatttggtgctgacgggctaccaggttgacaagaacaaagatgacgagctgacgggtttc

SEQ ID NO: 785 - DNA, 351 bp, synthetic construct
  source 1..351: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgaagcacatcaggatctgcatttctggggtacgaactactacatcaaggttcgtgcgggtgacaacaagtatatgcacctgaaagtgtttaagagcctgctgaccgaacataacaaaaaacatcaggaaagacatctggatcatgaagatttggtgctgacgggctaccaggttgacaagaacaaagatgacgagctgacgggtttc

SEQ ID NO: 786 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctaccgtttcggtccaccatacgttaaaggtacgaactactacatcaaggttcgtgcgggtgacaacaagtatatgcacctgaaagtgtttaagagcctgggttggaacgcattctcttctccagcagaagatttggtgctgacgggctaccaggttgacaagaacaaagatgacgagctgacgggtttc

SEQ ID NO: 787 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgcaaactctaaatggcattacgcattcggtacgaactactacatcaaggttcgtgcgggtgacaacaagtatatgcacctgaaagtgtttaagagcctggataacgcaatctacagaggtccaaccgaagatttggtgctgacgggctaccaggttgacaagaacaaagatgacgagctgacgggtttc

SEQ ID NO: 788 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaacgtccacatttcctgcagttctctggtacgaactactacatcaaggttcgtgcgggtgacaacaagtatatgcacctgaaagtgtttaagagcctgaacatcggtccagcaaaaaaacagctggaagatttggtgctgacgggctaccaggttgacaagaacaaagatgacgagctgacgggtttc

SEQ ID NO: 789 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccatcgtctgcatttcccaatctactacggtacgaactactacatcaaggttcgtgcgggtgacaacaagtatatgcacctgaaagtgtttaagagcctgcatttcaccctgccaacccagcatccagaagatttggtgctgacgggctaccaggttgacaagaacaaagatgacgagctgacgggtttc

SEQ ID NO: 790 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctactgggaacatcatcattacggtttcggtacgaactactacatcaaggttcgtgcgggtgacaacaagtatatgcacctgaaagtgtttaagagcctggcatggccagaaatcgatccaccagttgaagatttggtgctgacgggctaccaggttgacaagaacaaagatgacgagctgacgggtttc

SEQ ID NO: 791 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgaagaaggtaccccagaatgggcaccaggtacgaactactacatcaaggttcgtgcgggtgacaacaagtatatgcacctgaaagtgtttaagagcctgtggtctgatgttctgtacaacgatttcgaagatttggtgctgacgggctaccaggttgacaagaacaaagatgacgagctgacgggtttc

SEQ ID NO: 792 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgttcgtcatccacgttggttcttcatcggtacgaactactacatcaaggttcgtgcgggtgacaacaagtatatgcacctgaaagtgtttaagagcctgaaaaaacatggtgatgaagttaccctggaagatttggtgctgacgggctaccaggttgacaagaacaaagatgacgagctgacgggtttc

SEQ ID NO: 793 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcttcgttttcctggaagttaaatgggatggtacgaactactacatcaaggttcgtgcgggtgacaacaagtatatgcacctgaaagtgtttaagagcctgagaaaagaaagacatcagttcgatatcgaagatttggtgctgacgggctaccaggttgacaagaacaaagatgacgagctgacgggtttc

SEQ ID NO: 794 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgatgttaaaccaaaatggcagtggaccggtacgaactactacatcaaggttcgtgcgggtgacaacaagtatatgcacctgaaagtgtttaagagcctgttcgaaggtgaagatagagatgcagttgaagatttggtgctgacgggctaccaagttgacaagaacaaagatgacgagctgacgggtttc

SEQ ID NO: 795 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgcagcattctctgttcatccatcttctggtacgaactactacatcaaggttcgtgcgggtgacaacaagtatatgcacctgaaagtgtttaagagcctggaaaaataccataacatctggtacgttgaagatttggtgctgacgggctaccaggttgacaagaacaaagatgacgagctgacgggtttc

SEQ ID NO: 796 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcttcaccttcgttgatatccattggtacggtacgaactactacatcaaggttcgtgcgggtgacaacaagtatatgcacctgaaagtgtttaagagcctgagaaaaccaaaagatagacagtacaccgaagatttggtgctgacgggctaccaggttgacaagaacaaagatgacgagctgacgggtttc

SEQ ID NO: 797 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaatacgttgttcaggcatctaaagttggtacgaactactacatcaaggttcgtgcgggtgacaacaagtatatgcacctgaaagtgtttaagagcctgtacggtcagaaagatggtcagaaagaagaagatttggtgctgacgggctaccaggttgacaagaacaaagatgacgagctgacgggtttc

SEQ ID NO: 798 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgcacaggatctgtctgcattccagatcggtacgaactactacatcaaggttcgtgcgggtgacaacaagtatatgcacctgaaagtgtttaagagcctgatcttcgcaccactgaaaccatggagagaagatttggtgctgacgggctaccaggttgacaagaacaaagatgacgagctgacgggtttc

SEQ ID NO: 799 - DNA, 333 bp, synthetic construct; identical to SEQ ID NO: 797
  source 1..333: mol_type = other DNA

SEQ ID NO: 800 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaccagccacagccaccatggatccgtggtacgaactactacatcaaggttcgtgcgggtgacaacaagtatatgcacctgaaagtgtttaagagcctgaactggaactctccaaacctgacctgggaagatttggtgctgacgggctaccaggttgacaagaacaaagatgacgagctgacgggtttc

SEQ ID NO: 801 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcacccatctgaacaacaacgcatggaccggtacgaactactacatcaaggttcgtgcgggtgacaacaagtatatgcacctgaaagtgtttaagagcctgttcttccataacggtaaaatctacagagaagatttggtgctgacgggctaccaggttgacaagaacaaagatgacgagctgacgggtttc

SEQ ID NO: 802 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaatacgaaacccatttctactacgaaggtacgaactactacatcaaggttcgtgcgggtgacaacaagtatatgcacctgaaagtgtttaagagcctgccaaaaagatgggcatacaacgcatacgaagatttggtgctgacgggctaccaggttgacaagaacaaagatgacgagctgacgggtttc

SEQ ID NO: 803 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctggtctttcgcacaggttggttggcatggtacgaactactacatcaaggttcgtgcgggtgacaacaagtatatgcacctgaaagtgtttaagagcctgagaagattcgaacatctgccattctacgaagatttggtgctgacgggctaccaggttgacaagaacaaagatgacgagctgacgggtttc

SEQ ID NO: 804 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccatcatttcgcagaagttaaatggtacggtacgaactactacatcaaggttcgtgcgggtgacaacaagtatatgcacctgaaagtgtttaagagcctgaaaatcaccggttctaccctgaccttcgaagatttggtgctgacgggctaccaggttgacaagaacaaagatgacgagctgacgggtttc

SEQ ID NO: 805 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtgttccactggattctaacaaatggggtacgaactactacatcaaggttcgtgcgggtgacaacaagtatatgcacctgaaagtgtttaagagcctgttcagagttagagattactctttcctggaagatttggtgctgacgggctaccaggttgacaagaacaaagatgacgagctgacgggtttc

SEQ ID NO: 806 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgaaatccgtccagtttgggattggtctggtacgaactactacatcaaggttcgtgcgggtgacaacaagtatatgcacctgaaagtgtttaagagcctgttcgatcatgaatctaaaccaaccgcagaagatttggtgctgacgggctaccaggttgacaagaacaaagatgacgagctgacgggtttc

SEQ ID NO: 807 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaacgtcgtcatttcccacagtggcagggtacgaactactacatcaaggttcgtgcgggtgacaacaagtatatgcacctgaaagtgtttaagagcctgggtgatcatctgaaccagtacccaaacgaagatttggtgctgacgggctaccaggttgacaagaacaaagatgacgagctgacgggtttc

SEQ ID NO: 808 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcggtacccgtgatgaatggcactgggttggtacgaactactacatcaaggttcgtgcgggtgacaacaagtatatgcacctgaaagtgtttaagagcctgttcgttaaagatgatggtagagttgatgaagatttggtgctgacgggctaccaggttgacaagaacaaagatgacgagctgacgggtttc

SEQ ID NO: 809 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatccagcatatctggtgggatttcgttggtacgaactactacatcaaggttcgtgcgggtgacaacaagtatatgcacctgaaagtgtttaagagcctgtctagactggaaccagcaggtcatcaggaagatttggtgctgacgggctaccaggttgacaagaacaaagatgacgagctgacgggtttc

SEQ ID NO: 810 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgttcagcatccacagtggcattggaaaggtacgaactactacatcaaggttcgtgcgggtgacaacaagtatatgcacctgaaagtgtttaagagcctggttcagcagagagatgtttggatcaacgaagatttggtgctgacgggctaccaggttgacaagaacaaagatgacgagctgacgggtttc

SEQ ID NO: 811 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtaccaaagaaaccaaaaaatcttggggtacgaactactacatcaaggttcgtgcgggtgacaacaagtatatgcacctgaaagtgtttaagagcctgcagaaaatccatgcaatcttctacgttgaagatttggtgctgacgggctaccaggttgacaagaacaaagatgacgagctgacgggtttc

SEQ ID NO: 812 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtgaatacccacatttccagggttacggtacgaactactacatcaaggttcgtgcgggtgacaacaagtatatgcacctgaaagtgtttaagagcctgatcctgaacgcaggtggtatcagatgggaagatttggtgctgacgggctaccaggttgacaagaacaaagatgacgagctgacgggtttc

SEQ ID NO: 813 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgttcagcattctctgtggtacttccagggtacgaactactacatcaaggttcgtgcgggtgacaacaagtatatgcacctgaaagtgtttaagagcctgttcaaacatccagaagaaaacccagttgaagatttggtgctgacgggctaccaggttgacaagaacaaagatgacgagctgacgggtttc

SEQ ID NO: 814 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcggtgttaaacagacctggaactggaacggtacgaactactacatcaaggttcgtgcgggtgacaacaagtatatgcacctgaaagtgtttaagagcctgttcggtctgaacgatgatgaaccagttgaagatttggtgctgacgggctaccaggttgacaagaacaaagatgacgagctgacgggtttc

SEQ ID NO: 815 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcacccgtaaacatttcccacagtactggggtacgaactactacatcaaggttcgtgcgggtgacaacaagtatatgcacctgaaagtgtttaagagcctgaacgaaccactgccagttaccaacatcgaagatttggtgctgacgggctaccaggttgacaagaacaaagatgacgagctgacgggtttc

SEQ ID NO: 816 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaatactacgcaatcttcgattacaaaggtacgaactactacatcaaggttcgtgcgggtgacaacaagtatatgcacctgaaagtgtttaagagcctgttcaaagaaaaagttagaaactctatcgaagatttggtgctgacgggctaccaggttgacaagaacaaagatgacgagctgacgggtttc

SEQ ID NO: 817 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcggtcgtcaggttaaatgggcatggaccggtacgaactactacatcaaggttcgtgcgggtgacaacaagtatatgcacctgaaagtgtttaagagcctgttcaaacagcatcataacatctctgaagaagatttggtgctgacgggctaccaggttgacaagaacaaagatgacgagctgacgggtttc

SEQ ID NO: 818 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctctcatcatgcatctccatctcatatcggtacgaactactacatcaaggttcgtgcgggtgacaacaagtatatgcacctgaaagtgtttaagagcctgaccaccgatccaaactgggatgttcatgaagatttggtgctgacgggctaccaggttgacaagaacaaagatgacgagctgacgggtttc

SEQ ID NO: 819 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcggtatccgtgaagattggaactggtctggtacgaactactacatcaaggttcgtgcgggtgacaacaagtatatgcacctgaaagtgtttaagagcctgttcgaaccaaaaaaactggaaccagcagaagatttggtgctgacgggctaccaggttgacaagaacaaagatgacgagctgacgggtttc

SEQ ID NO: 820 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgcatgggattggctgccagcataccgtggtacgaactactacatcaaggttcgtgcgggtgacaacaagtatatgcacctgaaagtgtttaagagcctgttcaaattccagaacgaatctttcgcagaagatttggtgctgacgggctaccaggttgacaagaacaaagatgacgagctgacgggtttc

SEQ ID NO: 821 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatcaaccagcagcagatcaaacattctggtacgaactactacatcaaggttcgtgcgggtgacaacaagtatatgcacctgaaagtgtttaagagcctgtggcagcatatcccaccagatttctgggaagatttggtgctgacgggctaccaggttgacaagaacaaagatgacgagctgacgggtttc

SEQ ID NO: 822 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgcaggttggtctctgccaccacatctgggtacgaactactacatcaaggttcgtgcgggtgacaacaagtatatgcacctgaaagtgtttaagagcctgatcaaccatgcaggtcagaaatctgatgaagatttggtgctgacgggctaccaggttgacaagaacaaagatgacgagctgacgggtttc

SEQ ID NO: 823 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcggtggtcgtcatcgttggaactggtctggtacgaactactacatcaaggttcgtgcgggtgacaacaagtatatgcacctgaaagtgtttaagagcctgttcgaaccatctggtctggcagcagaagaagatttggtgctgacgggctaccaggttgacaagaacaaagatgacgagctgacgggtttc

SEQ ID NO: 824 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgaactggatggtgatcatacctggctgggtacgaactactacatcaaggttcgtgcgggtgacaacaagtatatgcacctgaaagtgtttaagagcctgagacagctgcatagaatctggtacttcgaagatttggtgctgacgggctaccaggttgacaagaacaaagatgacgagctgacgggtttc

SEQ ID NO: 825 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaacagctgatcatctggcattggaccggtacgaactactacatcaaggttcgtgcgggtgacaacaagtatatgcacctgaaagtgtttaagagcctgttcaaaaactctaactctcatctggaagaagatttggtgctgacgggctaccaggttgacaagaacaaagatgacgagctgacgggtttc

SEQ ID NO: 826 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtgaaccagcactgtggcatttccgtggtacgaactactacatcaaggttcgtgcgggtgacaacaagtatatgcacctgaaagtgtttaagagcctgaccttcaaagttgatacctggaaagttgaagatttggtgctgacgggctaccaggttgacaagaacaaagatgacgagctgacgggtttc

SEQ ID NO: 827 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcccacaggttcaggaaccatggtggatcggtacgaactactacatcaaggttcgtgcgggtgacaacaagtatatgcacctgaaagtgtttaagagcctgtacaaagcatacatccagggttggaacgaagatttggtgctgacgggctaccaggttgacaagaacaaagatgacgagctgacgggtttc

SEQ ID NO: 828 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcccaaccggtacccatcagtgggcagcaggtacgaactactacatcaaggttcgtgcgggtgacaacaagtatatgcacctgaaagtgtttaagagcctgtgggcatctctggaatctggtcagaacgaagatttggtgctgacgggctaccaggttgacaagaacaaagatgacgagctgacgggtttc

SEQ ID NO: 829 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcttccgtcagcatttcccaaacttctctggtacgaactactacatcaaggttcgtgcgggtgacaacaagtatatgcacctgaaagtgtttaagagcctgaacggtctggaagatccaacccaggatgaagatttggtgctgacgggctaccaggttgacaagaacaaagatgacgagctgacgggtttc

SEQ ID NO: 830 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccaggaatacgaagatcaggaatgggcaggtacgaactactacatcaaggttcgtgcgggtgacaacaagtatatgcacctgaaagtgtttaagagcctgcatctgtggaaccagcagatctacagagaagatttggtgctgacgggctaccaggttgacaagaacaaagatgacgagctgacgggtttc

SEQ ID NO: 831 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatcgcacatatccattggaaatacttcggtacgaactactacatcaaggttcgtgcgggtgacaacaagtatatgcacctgaaagtgtttaagagcctgcatctggcaaaaggtcagtggagagaagaagatttggtgctgacgggctaccaggttgacaagaacaaagatgacgagctgacgggtttc

SEQ ID NO: 832 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgattaccgtgcaatctggcattggtggggtacgaactactacatcaaggttcgtgcgggtgacaacaagtatatgcacctgaaagtgtttaagagcctgttccatgcagaaggtgatatctgggatgaagatttggtgctgacgggctaccaggttgacaagaacaaagatgacgagctgacgggtttc

SEQ ID NO: 833 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaacctgaaaccagatgttcagggttctggtacgaactactacatcaaggttcgtgcgggtgacaacaagtatatgcacctgaaagtgtttaagagcctgatcgttgttaaaagatactggtacgttgaagatttggtgctgacgggctaccaggttgacaagaacaaagatgacgagctgacgggtttc

SEQ ID NO: 834 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctctaccaaaaacctgtggcgttggtctggtacgaactactacatcaaggttcgtgcgggtgacaacaagtatatgcacctgaaagtgtttaagagcctgttcaacggtgcaggtggtccagattctgaagatttggtgctgacgggctaccaggttgacaagaacaaagatgacgagctgacgggtttc

SEQ ID NO: 835 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtgttcagggtctgtggaacttccagggtacgaactactacatcaaggttcgtgcgggtgacaacaagtatatgcacctgaaagtgtttaagagcctgggacatcatggtgatcatgaaaaatgggaagatttggtgctgacgggctaccaggttgacaagaacaaagatgacgagctgacgggtttc

SEQ ID NO: 836 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctacaaagttgataacaacaactaccagggtacgaactactacatcaaggttcgtgcgggtgacaacaagtatatgcacctgaaagtgtttaagagcctgatcacctggcagctgaaccagccaagagaagatttggtgctgacgggctaccaggttgacaagaacaaagatgacgagctgacgggtttc

SEQ ID NO: 837 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatcgaagaaccatctgcattctggtggggtacgaactactacatcaaggttcgtgcgggtgacaacaagtatatgcacctgaaagtgtttaagagcctgctgagagttagacattctgcatacagagaagatttggtgctgacgggctaccaggttgacaagaacaaagatgacgagctgacgggtttc

SEQ ID NO: 838 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgcatctttcccagaaatctcttggtacggtacgaactactacatcaaggttcgtgcgggtgacaacaagtatatgcacctgaaagtgtttaagagcctgaaaaactacccaaaaaccggtgttaccgaagatttggtgctgacgggctaccaggttgacaagaacaaagatgacgagctgacgggtttc

SEQ ID NO: 839 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatccatgaaccagaaatctggtggtacggtacgaactactacatcaaggttcgtgcgggtgacaacaagtatatgcacctgaaagtgtttaagagcctgcataaagcagatagacagaaagaaagagaagatttggtgctgacgggctaccaggttgacaagaacaaagatgacgagctgacgggtttc

SEQ ID NO: 840 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgattggggttggggtatcccatgggttggtacgaactactacatcaaggttcgtgcgggtgacaacaagtatatgcacctgaaagtgtttaagagcctgtggctgtacggtgttaaaagactgccagaagatttggtgctgacgggctaccaggttgacaagaacaaagatgacgagctgacgggtttc

SEQ ID NO: 841 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgttgggaactgtggtctttccagggtacgaactactacatcaaggttcgtgcgggtgacaacaagtatatgcacctgaaagtgtttaagagcctgtggcaggaacataccgattctgttgaagaagatttggtgctgacgggctaccaggttgacaagaacaaagatgacgagctgacgggtttc

SEQ ID NO: 842 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatcaaccagcatgaaaacttcaaacagggtacgaactactacatcaaggttcgtgcgggtgacaacaagtatatgcacctgaaagtgtttaagagcctgcatctgttccatggtctgtggtacatcgaagatttggtgctgacgggctaccaggttgacaagaacaaagatgacgagctgacgggtttc

SEQ ID NO: 843 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccatgatttcaccatccgttacccatggggtacgaactactacatcaaggttcgtgcgggtgacaacaagtatatgcacctgaaagtgtttaagagcctggaataccatgttaccaaaagagatcaggaagatttggtgctgacgggctaccaggttgacaagaacaaagatgacgagctgacgggtttc

SEQ ID NO: 844 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctgggatcgtccacgttggggttggaccggtacgaactactacatcaaggttcgtgcgggtgacaacaagtatatgcacctgaaagtgtttaagagcctgttcaaagaatcttctagaaacgatgaagaagatttggtgctgacgggctaccaggttgacaagaacaaagatgacgagctgacgggtttc

SEQ ID NO: 845 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgatcagggttggtgggaaaactgggcaggtacgaactactacatcaaggttcgtgcgggtgacaacaagtatatgcacctgaaagtgtttaagagcctgatctgggaaaacaacctgagaatccatgaagatttggtgctgacgggctaccaggttgacaagaacaaagatgacgagctgacgggtttc

SEQ ID NO: 846 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatctggcattctgaatggtcttacaccggtacgaactactacatcaaggttcgtgcgggtgacaacaagtatatgcacctgaaagtgtttaagagcctgttcaccagacattctgaaccatgggaagaagatttggtgctgacgggctaccaggttgacaagaacaaagatgacgagctgacgggtttc

SEQ ID NO: 847 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcccacagtggccaggtacgaactactacatcaaggttcgtgcgggtgacaacaagtatatgcacctgaaagtgtttaagagcctggatctgcagccaagagaagttttccaggaagatttggtgctgacgggctaccaggttgacaagaacaaagatgacgagctgacgggtttc

SEQ ID NO: 848 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaagcatacgttatctggtcttacaaaggtacgaactactacatcaaggttcgtgcgggtgacaacaagtatatgcacctgaaagtgtttaagagcctgttctacggtgaaccaagaggtaaaggtgaagatttggtgctgacgggctaccaggttgacaagaacaaagatgacgagctgacgggtttc

SEQ ID NO: 849 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcttctggcagctgcatcgttacggtttcggtacgaactactacatcaaggttcgtgcgggtgacaacaagtatatgcacctgaaagtgtttaagagcctggaatggaacagactgtctccactgtgggaagatttggtgctgacgggctaccaggttgacaagaacaaagatgacgagctgacgggtttc

SEQ ID NO: 850 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatctaccatgaactgtggaacttctacggtacgaactactacatcaaggttcgtgcgggtgacaacaagtatatgcacctgaaagtgtttaagagcctggataacaacggtgatggtaactggcaggaagatttggtgctgacgggctaccaggttgacaagaacaaagatgacgagctgacgggtttc

SEQ ID NO: 851 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgatccgtcatctgtggtcttggtctggtacgaactactacatcaaggttcgtgcgggtgacaacaagtatatgcacctgaaagtgtttaagagcctgttcgaaggtgaaaaccatcagatcgatgaagatttggtgctgacgggctaccaggttgacaagaacaaagatgacgagctgacgggtttc

SEQ ID NO: 852 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaacgatggtaaatctctggttctggcaggtacgaactactacatcaaggttcgtgcgggtgacaacaagtatatgcacctgaaagtgtttaagagcctgagactgtgggatgatagagtttctggtgaagatttggtgctgacgggctaccaggttgacaagaacaaagatgacgagctgacgggtttc

SEQ ID NO: 853 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgatgttcgtggtcagtgggcatggaccggtacgaactactacatcaaggttcgtgcgggtgacaacaagtatatgcacctgaaagtgtttaagagcctgttccagccaagaaccaaagcaaccgaagaagatttggtgctgacgggctaccaggttgacaagaacaaagatgacgagctgacgggtttc

SEQ ID NO: 854 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaacgtctgcatctgccacagtacccaggtacgaactactacatcaaggttcgtgcgggtgacaacaagtatatgcacctgaaagtgtttaagagcctgggtaacatcgaaccaatcaccgatctggaagatttggtgctgacgggctaccaggttgacaagaacaaagatgacgagctgacgggtttc

SEQ ID NO: 855 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgcagttcatgcaccacagtacgcaccaggtacgaactactacatcaaggttcgtgcgggtgacaacaagtatatgcacctgaaagtgtttaagagcctgtggtctgaaaaagttgcagttttcccagaagatttggtgctgacgggctaccaggttgacaagaacaaagatgacgagctgacgggtttc

SEQ ID NO: 856 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgatttccgtgttgcatggcattgggttggtacgaactactacatcaaggttcgtgcgggtgacaacaagtatatgcacctgaaagtgtttaagagcctgttcaaccatagaccaggtaaaccagatgaagatttggtgctgacgggctaccaggttgacaagaacaaagatgacgagctgacgggtttc

SEQ ID NO: 857 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgggttggtggttcggtatcaaccagggtacgaactactacatcaaggttcgtgcgggtgacaacaagtatatgcacctgaaagtgtttaagagcctggcacatctgcagtacgaatggggtggtgaagatttggtgctgacgggctaccaggttgacaagaacaaagatgacgagctgacgggtttc

SEQ ID NO: 858 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaccaggaacatttccgtcgtttcatcggtacgaactactacatcaaggttcgtgcgggtgacaacaagtatatgcacctgaaagtgtttaagagcctgaacccatggctgcatccacaggatttcgaagatttggtgctgacgggctaccaggttgacaagaacaaagatgacgagctgacgggtttc

SEQ ID NO: 859 - DNA, 327 bp, synthetic construct
  source 1..327: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacatcgatgatgatctgtggtctggtacgaactactacatcaaggttcgtgcgggtgacaacaagtatatgcacctgaaagtgtttaagagcctgatcctgtggccaaccaaaccagaaagagaagatttggtgctgacgggctaccaggttgacaagaacaaagatgacgagctgacgggtttc

SEQ ID NO: 860 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgaccagtggggtgttgatctggtttacaccggtacgaactactacatcaaggttcgtgcgggtgacaacaagtatatgcacctgaaagtgtttaagagcctgatcgttaaaagagcagttatcctgtgggaagatttggtgctgacgggctaccaggttgacaagaacaaagatgacgagctgacgggtttc

SEQ ID NO: 861 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacaaagtttacctggtttgggcatggaccggtacgaactactacatcaaggttcgtgcgggtgacaacaagtatatgcacctgaaagtgtttaagagcctgttccagaaaggttctaacaaaccagaagaagatttggtgctgacgggctaccaggttgacaagaacaaagatgacgagctgacgggtttc

SEQ ID NO: 862 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacaccatccatccacaggcaaaatactggggtacgaactactacatcaaggttcgtgcgggtgacaacaagtatatgcacctgaaagtgtttaagagcctgtggaaaaacctgctgaaaccaatccatgaagatttggtgctgacgggctaccaggttgacaagaacaaagatgacgagctgacgggtttc

SEQ ID NO: 863 - DNA, 330 bp, synthetic construct
  source 1..330: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacttcaaccagtctcgtcatggtcatggtacgaactactacatcaaggttcgtgcgggtgacaacaagtatatgcacctgaaagtgtttaagagcctggaactgctgcatggtctgtggtgggttgaagatttggtgctgacgggctaccaggttgacaagaacaaagatgacgagctgacgggtttc

SEQ ID NO: 864 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacgaactggatgatgcagttaaatactggggtacgaactactacatcaaggttcgtgcgggtgacaacaagtatatgcacctgaaagtgtttaagagcctgtggtggaaaaccatctctgcagatttcgaagatttggtgctgacgggctaccaggttgacaagaacaaagatgacgagctgacgggtttc

SEQ ID NO: 865 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgactgggcaatctggcgtgataaaaaagttggtacgaactactacatcaaggttcgtgcgggtgacaacaagtatatgcacctgaaagtgtttaagagcctgccaatcttcgaaatcgttagagaaggtgaagatttggtgctgacgggctaccaggttgacaagaacaaagatgacgagctgacgggtttc

SEQ ID NO: 866 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacacctggggtgaacgtctgcattacccaggtacgaactactacatcaaggttcgtgcgggtgacaacaagtatatgcacctgaaagtgtttaagagcctgtggcataaaccaaaagataacatcatcgaagatttggtgctgacgggctaccaggttgacaagaacaaagatgacgagctgacgggtttc

SEQ ID NO: 867 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgaccataccgatgttgattggcatttctggggtacgaactactacatcaaggttcgtgcgggtgacaacaagtatatgcacctgaaagtgtttaagagcctggcaccagcagcaccaccatgggaattcgaagatttggtgctgacgggctaccaggttgacaagaacaaagatgacgagctgacgggtttc

SEQ ID NO: 868 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgaccatcatcatcgtttcgcaaaatggtggggtacgaactactacatcaaggttcgtgcgggtgacaacaagtatatgcacctgaaagtgtttaagagcctgtggttcaacgaaaacagagaagataccgaagatttggtgctgacgggctaccaggttgacaagaacaaagatgacgagctgacgggtttc

SEQ ID NO: 869 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgaccgtaaagcagcagttgttgcacgtcatggtacgaactactacatcaaggttcgtgcgggtgacaacaagtatatgcacctgaaagtgtttaagagcctgtggccattcctggttatcgaaaaatacgaagatttggtgctgacgggctaccaggttgacaagaacaaagatgacgagctgacgggtttc

SEQ ID NO: 870 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacttctctttcccactggttcattggtacggtacgaactactacatcaaggttcgtgcgggtgacaacaagtatatgcacctgaaagtgtttaagagcctgagattcagagatggtggtcatcatgttgaagatttggtgctgacgggctaccaggttgacaagaacaaagatgacgagctgacgggtttc

SEQ ID NO: 871 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacaacgcacatccactggttaaaaactggggtacgaactactacatcaaggttcgtgcgggtgacaacaagtatatgcacctgaaagtgtttaagagcctgtggagaaacgatacctcttctatctacgaagatttggtgctgacgggctaccaggttgacaagaacaaagatgacgagctgacgggtttc

SEQ ID NO: 872 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacgaaacctctgaaaacgttatccagtggggtacgaactactacatcaaggttcgtgcgggtgacaacaagtatatgcacctgaaagtgtttaagagcctgtggccagcagatatcctgacccagcatgaagatttggtgctgacgggctaccaggttgacaagaacaaagatgacgagctgacgggtttc

SEQ ID NO: 873 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacttctctgatctggaatgggaaggtcagggtacgaactactacatcaaggttcgtgcgggtgacaacaagtatatgcacctgaaagtgtttaagagcctggaaaaaaagtggggttggatctacatcgaagatttggtgctgacgggctaccaggttgacaagaacaaagatgacgagctgacgggtttc

SEQ ID NO: 874 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacttcaccgatatccattggtctacctggggtacgaactactacatcaaggttcgtgcgggtgacaacaagtatatgcacctgaaagtgtttaagagcctgatcccagcatggcagcagatccatgttgaagatttggtgctgacgggctaccaggttgacaagaacaaagatgacgagctgacgggtttc

SEQ ID NO: 875 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgaccgtgaacatgcatctgttaaattctggggtacgaactactacatcaaggttcgtgcgggtgacaacaagtatatgcacctgaaagtgtttaagagcctgtggctggaagcaaccccacatacccaggaagatttggtgctgacgggctaccaggttgacaagaacaaagatgacgagctgacgggtttc

SEQ ID NO: 876 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacatctctggtgttggtctggttccaaacggtacgaactactacatcaaggttcgtgcgggtgacaacaagtatatgcacctgaaagtgtttaagagcctgaactggaactacccacagcagccattcgaagatttggtgctgacgggctaccaggttgacaagaacaaagatgacgagctgacgggtttc

SEQ ID NO: 877 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgaccatacccagccaaaagcactggattggggtacgaactactacatcaaggttcgtgcgggtgacaacaagtatatgcacctgaaagtgtttaagagcctgtggggttctatcctgaccgatggtccagaagatttggtgctgacgggctaccaggttgacaagaacaaagatgacgagctgacgggtttc

SEQ ID NO: 878 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacatccaggatcgtctgggtggtttcatcggtacgaactactacatcaaggttcgtgcgggtgacaacaagtatatgcacctgaaagtgtttaagagcctgcatttctacagaggtatcctgtacagagaagatttggtgctgacgggctaccaggttgacaagaacaaagatgacgagctgacgggtttc

SEQ ID NO: 879 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgaccgtttctaccatcagtggcatttcctgggtacgaactactacatcaaggttcgtgcgggtgacaacaagtatatgcacctgaaagtgtttaagagcctggatctgagagcaccaagaaacgataacgaagatttggtgctgacgggctaccaggttgacaagaacaaagatgacgagctgacgggtttc

SEQ ID NO: 880 - DNA, 330 bp, synthetic construct
  source 1..330: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgactgggttacccgtcatcataccctggttggtacgaactactacatcaaggttcgtgcgggtgacaacaagtatatgcacctgaaagtgtttaagagcctgccaccattcaaacattggcatctggaagatttggtgctgacgggctaccaggttgacaagaacaaagatgacgagctgacgggtttc

SEQ ID NO: 881 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgaccagtcttctgatctggcaaaactgtggggtacgaactactacatcaaggttcgtgcgggtgacaacaagtatatgcacctgaaagtgtttaagagcctgtggaacctgaactctgataaatcttctgaagatttggtgctgacgggctaccaggttgacaagaacaaagatgacgagctgacgggtttc

SEQ ID NO: 882 - DNA, 333 bp, synthetic construct; identical to SEQ ID NO: 879
  source 1..333: mol_type = other DNA

SEQ ID NO: 883 - DNA, 330 bp, synthetic construct; identical to SEQ ID NO: 880
  source 1..330: mol_type = other DNA

SEQ ID NO: 884 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgaccatgaagttcgtaaaacctacgaattcggtacgaactactacatcaaggttcgtgcgggtgacaacaagtatatgcacctgaaagtgtttaagagcctgtggttcggtggttggccactggcacaggaagatttggtgctgacgggctaccaggttgacaagaacaaagatgacgagctgacgggtttc

SEQ ID NO: 885 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacgttatctctatcccactggtttggaacggtacgaactactacatcaaggttcgtgcgggtgacaacaagtatatgcacctgaaagtgtttaagagcctgatcttccaggaatactacccacatagagaagatttggtgctgacgggctaccaggttgacaagaacaaagatgacgagctgacgggtttc

SEQ ID NO: 886 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgactcttgggttttcaaaccattctaccacggtacgaactactacatcaaggttcgtgcgggtgacaacaagtatatgcacctgaaagtgtttaagagcctgtctggtccagaattcttctctagacatgaagatttggtgctgacgggctaccaggttgacaagaacaaagatgacgagctgacgggtttc

SEQ ID NO: 887 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacccagaagtttctatcacccgttggcagggtacgaactactacatcaaggttcgtgcgggtgacaacaagtatatgcacctgaaagtgtttaagagcctgtacgaaacccatggtaacatcatcaccgaagatttggtgctgacgggctaccaggttgacaagaacaaagatgacgagctgacgggtttc

SEQ ID NO: 888 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacggtaacttcgcacatgaacgttggcagggtacgaactactacatcaaggttcgtgcgggtgacaacaagtatatgcacctgaaagtgtttaagagcctgtacatcgcaagagatttcgattggaccgaagatttggtgctgacgggctaccaggttgacaagaacaaagatgacgagctgacgggtttc

SEQ ID NO: 889 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacgttatccatatcccacatcagtggcatggtacgaactactacatcaaggttcgtgcgggtgacaacaagtatatgcacctgaaagtgtttaagagcctgatcttccatgaaccaaaaccaccaagagaagatttggtgctgacgggctaccaggttgacaagaacaaagatgacgagctgacgggtttc

SEQ ID NO: 890 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgacaacctggtttactaccagcgtgattggggtacgaactactacatcaaggttcgtgcgggtgacaacaagtatatgcacctgaaagtgtttaagagcctggaaccaggtagatggagatggccaaaagaagatttggtgctgacgggctaccaggttgacaagaacaaagatgacgagctgacgggtttc

SEQ ID NO: 891 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgaccaggttctgtgggaaatcttcgaacatggtacgaactactacatcaaggttcgtgcgggtgacaacaagtatatgcacctgaaagtgtttaagagcctgttccatcagtggtctccagcactgcatgaagatttggtgctgacgggctaccaggttgacaagaacaaagatgacgagctgacgggtttc

SEQ ID NO: 892 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcacccgtctgcatttcccacagtaccagggtacgaactactacatcaaggttcgtgcgggtgacaacaagtatatgcacctgaaagtgtttaagagcctggaaatcgaagttaacccatacgcaaacgaagatttggtgctgacgggctaccaggttgacaagaacaaagatgacgagctgacgggtttc

SEQ ID NO: 893 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctacgaaccacatcatccatacccatggggtacgaactactacatcaaggttcgtgcgggtgacaacaagtatatgcacctgaaagtgtttaagagcctgcagtaccatccaccatacgaaagattcgaagatttggtgctgacgggctaccaggttgacaagaacaaagatgacgagctgacgggtttc

SEQ ID NO: 894 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcacccaggcaatcccatggcgtgaatggggtacgaactactacatcaaggttcgtgcgggtgacaacaagtatatgcacctgaaagtgtttaagagcctgatcccagaatggccaaaagatgcatctgaagatttggtgctgacgggctaccaggttgacaagaacaaagatgacgagctgacgggtttc

SEQ ID NO: 895 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcccagaagttgataaagcagaatggtggggtacgaactactacatcaaggttcgtgcgggtgacaacaagtatatgcacctgaaagtgtttaagagcctgatcagatacggtctgagacatgaaagagaagatttggtgctgacgggctaccaggttgacaagaacaaagatgacgagctgacgggtttc

SEQ ID NO: 896 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccagcgtgttcatttcccagattggccaggtacgaactactacatcaaggttcgtgcgggtgacaacaagtatatgcacctgaaagtgtttaagagcctgaacaaagaaaaccaggatgaagaagatgaagatttggtgctgacgggctaccaggttgacaagaacaaagatgacgagctgacgggtttc

SEQ ID NO: 897 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcccacagccagaatggaacgaatggggtgttacgaactactacatcaaggttcgtgcgggtgacaacaagtatatgcacctgaaagtgtttaagagcctgatcttctggaacgaaaaactgatcagagaagatttggtgctgacgggctaccaggttgacaagaacaaagatgacgagctgacgggtttc

SEQ ID NO: 898 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaccagtgggaatggccagttgatctgggtacgaactactacatcaaggttcgtgcgggtgacaacaagtatatgcacctgaaagtgtttaagagcctgaaagtttacagaacccatcatatcttcgaagatttggtgctgacgggctaccaggttgacaagaacaaagatgacgagctgacgggtttc

SEQ ID NO: 899 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgaacgttaccatttcccacagttcaccggtacgaactactacatcaaggttcgtgcgggtgacaacaagtatatgcacctgaaagtgtttaagagcctgaaccatgatcaggttagatggaacctggaagatttggtgctgacgggctaccaggttgacaagaacaaagatgacgagctgacgggtttc

SEQ ID NO: 900 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgtactctgaatctcgtgattacaaaggtacgaactactacatcaaggttcgtgcgggtgacaacaagtatatgcacctgaaagtgtttaagagcctgcatgcatacaaaggttctatctacagagaagatttggtgctgacgggctaccaggttgacaagaacaaagatgacgagctgacgggtttc

SEQ ID NO: 901 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcttctggttcgaagatatccagtggctgggtacgaactactacatcaaggttcgtgcgggtgacaacaagtatatgcacctgaaagtgtttaagagcctgagattcagagaagatgttgatatcagagaagatttggtgctgacgggctaccaggttgacaagaacaaagatgacgagctgacgggtttc

SEQ ID NO: 902 - DNA, 330 bp, synthetic construct
  source 1..330: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgcagtttacccaaccttcaaatacggtacgaactactacatcaaggttcgtgcgggtgacaacaagtatatgcacctgaaagtgtttaagagcctgaaagttgaatacctggatttcatcaccgaagatttggtgctgacgggctaccaggttgacaagaacaaagatgacgagctgacgggtttc

SEQ ID NO: 903 - DNA, 330 bp, synthetic construct
  source 1..330: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgttctgcgttggcagtggggttacctgggtacgaactactacatcaaggttcgtgcgggtgacaacaagtatatgcacctgaaagtgtttaagagcctgcagctgctgtacgaagataacgatgaagatttggtgctgacgggctaccaggttgacaagaacaaagatgacgagctgacgggtttc

SEQ ID NO: 904 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgtggccactgcagccaaaaaactggggtacgaactactacatcaaggttcgtgcgggtgacaacaagtatatgcacctgaaagtgtttaagagcctgggttggaactacctggaagcaatcgcagaagatttggtgctgacgggctaccaggttgacaagaacaaagatgacgagctgacgggtttc

SEQ ID NO: 905 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgataacgttcgtcatggttggctgtctggtacgaactactacatcaaggttcgtgcgggtgacaacaagtatatgcacctgaaagtgtttaagagcctgttcgcactgctgtggtctgcatacggtgaagatttggtgctgacgggctaccaggttgacaagaacaaagatgacgagctgacgggtttc

SEQ ID NO: 906 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcggtctgatccagccaccatacgcacagggtacgaactactacatcaaggttcgtgcgggtgacaacaagtatatgcacctgaaagtgtttaagagcctgggttggcagtacatcgatctgttccatgaagatttggtgctgacgggctaccaggttgacaagaacaaagatgacgagctgacgggtttc

SEQ ID NO: 907 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcaggttggtgtttgggcatacgaaggtacgaactactacatcaaggttcgtgcgggtgacaacaagtatatgcacctgaaagtgtttaagagcctgggtagaaccgttgaaggtggtagatgggaagatttggtgctgacgggctaccaggttgacaagaacaaagatgacgagctgacgggtttc

SEQ ID NO: 908 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgatctgcataccctgaaatctggtgttggtacgaactactacatcaaggttcgtgcgggtgacaacaagtatatgcacctgaaagtgtttaagagcctgtacatcaaacatcatctgtggtggatcgaagatttggtgctgacgggctaccaggttgacaagaacaaagatgacgagctgacgggtttc

SEQ ID NO: 909 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctcttggtacaaactgtggtcttactctggtacgaactactacatcaaggttcgtgcgggtgacaacaagtatatgcacctgaaagtgtttaagagcctgttcgaaggtagacataaagatccagaagaagatttggtgctgacgggctaccaggttgacaagaacaaagatgacgagctgacgggtttc

SEQ ID NO: 910 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgattggccatctcatgcaaaattctggggtacgaactactacatcaaggttcgtgcgggtgacaacaagtatatgcacctgaaagtgtttaagagcctgttcctgccagataaagatgatccattcgaagatttggtgctgacgggctaccaggttgacaagaacaaagatgacgagctgacgggtttc

SEQ ID NO: 911 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgcataccagtctccaccttaccatttcggtacgaactactacatcaaggttcgtgcgggtgacaacaagtatatgcacctgaaagtgtttaagagcctgagacagatcgcaggttacatcgcaatcgaagatttggtgctgacgggctaccaggttgacaagaacaaagatgacgagctgacgggtttc

SEQ ID NO: 912 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctacaaaaacgttggtccatgggttaacggtacgaactactacatcaaggttcgtgcgggtgacaacaagtatatgcacctgaaagtgtttaagagcctgtacagaaccaacagaggtcatgaaagagaagatttggtgctgacgggctaccaggttgacaagaacaaagatgacgagctgacgggtttc

SEQ ID NO: 913 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctctaaacgttctgcatggcattggaccggtacgaactactacatcaaggttcgtgcgggtgacaacaagtatatgcacctgaaagtgtttaagagcctgttctggaacgcaccagatgatagacaggaagatttggtgctgacgggctaccaggttgacaagaacaaagatgacgagctgacgggtttc

SEQ ID NO: 914 - DNA, 330 bp, synthetic construct
  source 1..330: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgttcatatcaccgttgaatgggaaggtacgaactactacatcaaggttcgtgcgggtgacaacaagtatatgcacctgaaagtgtttaagagcctgatctaccagagacatacccagtctagagaagatttggtgctgacgggctaccaggttgacaagaacaaagatgacgagctgacgggtttc

SEQ ID NO: 915 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtgattacgatccattcccatacaccggtacgaactactacatcaaggttcgtgcgggtgacaacaagtatatgcacctgaaagtgtttaagagcctgtggagaggtccaggtccatacgcaatcgaagatttggtgctgacgggctaccaggttgacaagaacaaagatgacgagctgacgggtttc

SEQ ID NO: 916 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaaaccctgggtgtttggcattggtctggtacgaactactacatcaaggttcgtgcgggtgacaacaagtatatgcacctgaaagtgtttaagagcctgttcgaacagcatgcagaaaacatcgaagaagatttggtgctgacgggctaccaggttgacaagaacaaagatgacgagctgacgggtttc

SEQ ID NO: 917 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtgaagttggtctgtggcgtttccagggtacgaactactacatcaaggttcgtgcgggtgacaacaagtatatgcacctgaaagtgtttaagagcctgtacatcggttacgcaggtgatgcacaggaagatttggtgctgacgggctaccaggttgacaagaacaaagatgacgagctgacgggtttc

SEQ ID NO: 918 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctgggaagaagttaactggcgtaactggggtacgaactactacatcaaggttcgtgcgggtgacaacaagtatatgcacctgaaagtgtttaagagcctggttccaatccatgatcatctgagaaacgaagatttggtgctgacgggctaccaggttgacaagaacaaagatgacgagctgacgggtttc

SEQ ID NO: 919 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtgcacagctgcgtccatgggttgcaggtacgaactactacatcaaggttcgtgcgggtgacaacaagtatatgcacctgaaagtgtttaagagcctgggttggaacgaagaagcaagaccaccagaagatttggtgctgacgggctaccaggttgacaagaacaaagatgacgagctgacgggtttc

SEQ ID NO: 920 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctacccatggtcttggaacggtcattacggtacgaactactacatcaaggttcgtgcgggtgacaacaagtatatgcacctgaaagtgtttaagagcctgatcaaatacccattcgcaagagcagaagaagatttggtgctgacgggctaccaggttgacaagaacaaagatgacgagctgacgggtttc

SEQ ID NO: 921 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcccacagatcggtgcaccatacgttaaaggtacgaactactacatcaaggttcgtgcgggtgacaacaagtatatgcacctgaaagtgtttaagagcctgggttggaactctaacatcggtgttctggaagatttggtgctgacgggctaccaggttgacaagaacaaagatgacgagctgacgggtttc

SEQ ID NO: 922 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctggcgtaccggtcagaaatggcaggttggtacgaactactacatcaaggttcgtgcgggtgacaacaagtatatgcacctgaaagtgtttaagagcctggcaggtcattacgaagttcatatcggtgaagatttggtgctgacgggctaccaggttgacaagaacaaagatgacgagctgacgggtttc

SEQ ID NO: 923 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatccagcagctgtacaaacagacctggggtacgaactactacatcaaggttcgtgcgggtgacaacaagtatatgcacctgaaagtgtttaagagcctggattaccagcattggccatacgatttcgaagatttggtgctgacgggctaccaggttgacaagaacaaagatgacgagctgacgggtttc

SEQ ID NO: 924 - DNA, 327 bp, synthetic construct
  source 1..327: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatctactacccaatctgggatttccatggtacgaactactacatcaaggttcgtgcgggtgacaacaagtatatgcacctgaaagtgtttaagagcctgttctggggtgatctgaaaaccgaagatttggtgctgacgggctaccaggttgacaagaacaaagatgacgagctgacgggtttc

SEQ ID NO: 925 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgataacgttgcaaacatcgttccacatggtacgaactactacatcaaggttcgtgcgggtgacaacaagtatatgcacctgaaagtgtttaagagcctgggttggaactacgaacataaatggccagaagatttggtgctgacgggctaccaggttgacaagaacaaagatgacgagctgacgggtttc

SEQ ID NO: 926 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccatgttcattgggcatggaactacctgggtacgaactactacatcaaggttcgtgcgggtgacaacaagtatatgcacctgaaagtgtttaagagcctgagaaacttctacaccttcccacatgaagaagatttggtgctgacgggctaccaggttgacaagaacaaagatgacgagctgacgggtttc

SEQ ID NO: 927 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcggtccatggtacttcggttctgcatggggtacgaactactacatcaaggttcgtgcgggtgacaacaagtatatgcacctgaaagtgtttaagagcctgtgggaaaacttccataaactggaaaaagaagatttggtgctgacgggctaccaggttgacaagaacaaagatgacgagctgacgggtttc

SEQ ID NO: 928 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgttctggaacgtccaccatacgttaaaggtacgaactactacatcaaggttcgtgcgggtgacaacaagtatatgcacctgaaagtgtttaagagcctgaactgggaaggtgaaccatctggtgttgaagatttggtgctgacgggctaccaggttgacaagaacaaagatgacgagctgacgggtttc

SEQ ID NO: 929 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcttcaccttctcttctatcggttggtggggtacgaactactacatcaaggttcgtgcgggtgacaacaagtatatgcacctgaaagtgtttaagagcctgagacagctgctggataaaccaagatacgaagatttggtgctgacgggctaccaggttgacaagaacaaagatgacgagctgacgggtttc

SEQ ID NO: 930 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatctctgcatctgaaatccgttggtacggtacgaactactacatcaaggttcgtgcgggtgacaacaagtatatgcacctgaaagtgtttaagagcctgaaagcattcgcaaacgattactacaaagaagatttggtgctgacgggctaccaagttgacaagaacaaagatgacgagctgacgggtttc

SEQ ID NO: 931 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtatctggggtttctgggcattccagggtacgaactactacatcaaggttcgtgcgggtgacaacaagtatatgcacctgaaagtgtttaagagcctgggtcatgaatctggtcagaacctgtgggaagatttggtgctgacgggctaccaggttgacaagaacaaagatgacgagctgacgggtttc

SEQ ID NO: 932 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaacgaagcagaatcttctcgtttcctgggtacgaactactacatcaaggttcgtgcgggtgacaacaagtatatgcacctgaaagtgtttaagagcctgcatgatttcaccctgaaatggaaattcgaagatttggtgctgacgggctaccaggttgacaagaacaaagatgacgagctgacgggtttc

SEQ ID NO: 933 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgttgcaggtctgaaatggcattgggaaggtacgaactactacatcaaggttcgtgcgggtgacaacaagtatatgcacctgaaagtgtttaagagcctgatctacgcatctcagtacgattacagagaagatttggtgctgacgggctaccaggttgacaagaacaaagatgacgagctgacgggtttc

SEQ ID NO: 934 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctaccgtaacaaacagtggtacaacgaaggtacgaactactacatcaaggttcgtgcgggtgacaacaagtatatgcacctgaaagtgtttaagagcctgaaaatcggttacctggaattcgttctggaagatttggtgctgacgggctaccaggttgacaagaacaaagatgacgagctgacgggtttc

SEQ ID NO: 935 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctggttcgatacctggccagttccagaaggtacgaactactacatcaaggttcgtgcgggtgacaacaagtatatgcacctgaaagtgtttaagagcctgaactggaacgatcatctgtctacctacgaagatttggtgctgacgggctaccaggttgacaagaacaaagatgacgagctgacgggtttc

SEQ ID NO: 936 - DNA, 336 bp, synthetic construct
  source 1..336: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctacgcaaacatcggtccatggctgtacggtacgaactactacatcaaggttcgtgcgggtgacaacaagtatatgcacctgaaagtgtttaagagcctgttcaaacatagacagcagctgcattacaccgaagatttggtgctgacgggctaccaggttgacaagaacaaagatgacgagctgacgggtttc

SEQ ID NO: 937 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccatcagttccaggaaatcaaatggttcggtacgaactactacatcaaggttcgtgcgggtgacaacaagtatatgcacctgaaagtgtttaagagcctgttccatagaaccccaggttctaccaccgaagatttggtgctgacgggctaccaggttgacaagaacaaagatgacgagctgacgggtttc

SEQ ID NO: 938 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatcgatcatcagaaccatccaaccgaaggtacgaactactacatcaaggttcgtgcgggtgacaacaagtatatgcacctgaaagtgtttaagagcctgtggccatggctgcattctaccgaacaggaagatttggtgctgacgggctaccaggttgacaagaacaaagatgacgagctgacgggtttc

SEQ ID NO: 939 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctctgttttccagtacacctacaaattcggtacgaactactacatcaaggttcgtgcgggtgacaacaagtatatgcacctgaaagtgtttaagagcctgaacaacaaaccaccacataacgatctggaagatttggtgctgacgggctaccaggttgacaagaacaaagatgacgagctgacgggtttc

SEQ ID NO: 940 - DNA, 333 bp, synthetic construct; identical to SEQ ID NO: 910
  source 1..333: mol_type = other DNA

SEQ ID NO: 941 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcaggttaccatctggcatttcgaaggtacgaactactacatcaaggttcgtgcgggtgacaacaagtatatgcacctgaaagtgtttaagagcctgggtagaaccgaagttgaaggttctttcgaagatttggtgctgacgggctaccaggttgacaagaacaaagatgacgagctgacgggtttc

SEQ ID NO: 942 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctggcgtcgttggttcccacagtggccaggtacgaactactacatcaaggttcgtgcgggtgacaacaagtatatgcacctgaaagtgtttaagagcctgccagaagcagatgcagatgatccaaaagaagatttggtgctgacgggctaccaggttgacaagaacaaagatgacgagctgacgggtttc

SEQ ID NO: 943 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcttctggtacccagaaatcggttggctgggtacgaactactacatcaaggttcgtgcgggtgacaacaagtatatgcacctgaaagtgtttaagagcctgagatctagatacccaaactacaccgcagaagatttggtgctgacgggctaccaggttgacaagaacaaagatgacgagctgacgggtttc

SEQ ID NO: 944 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatctctgaagattcttggtggccacatggtacgaactactacatcaaggttcgtgcgggtgacaacaagtatatgcacctgaaagtgtttaagagcctgaactggttccatcagccatggcagagagaagatttggtgctgacgggctaccaggttgacaagaacaaagatgacgagctgacgggtttc

SEQ ID NO: 945 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgatcatctgccacatgcaaaatactggggtacgaactactacatcaaggttcgtgcgggtgacaacaagtatatgcacctgaaagtgtttaagagcctgtggtaccagccaagatggaaagttgaagaagatttggtgctgacgggctaccaggttgacaagaacaaagatgacgagctgacgggtttc

SEQ ID NO: 946 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcttcaaaaccgatgatgcacgttggatcggtacgaactactacatcaaggttcgtgcgggtgacaacaagtatatgcacctgaaagtgtttaagagcctgttcaactacaacaaaaccgaacataccgaagatttggtgctgacgggctaccaggttgacaagaacaaagatgacgagctgacgggtttc

SEQ ID NO: 947 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgcagggttctcgtctgccaatcgaaggtacgaactactacatcaaggttcgtgcgggtgacaacaagtatatgcacctgaaagtgtttaagagcctgggttggcataacgttaccctgccaccagaagatttggtgctgacgggctaccaggttgacaagaacaaagatgacgagctgacgggtttc

SEQ ID NO: 948 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaagcatacaaagttttcaactacatcggtacgaactactacatcaaggttcgtgcgggtgacaacaagtatatgcacctgaaagtgtttaagagcctgttcaaatgggataacggtatctctgatgaagatttggtgctgacgggctaccaggttgacaagaacaaagatgacgagctgacgggtttc

SEQ ID NO: 949 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgaaaaccaacccatctccatctgatggtacgaactactacatcaaggttcgtgcgggtgacaacaagtatatgcacctgaaagtgtttaagagcctggcagttttcagacagagactgggtgttgaagatttggtgctgacgggctaccaggttgacaagaacaaagatgacgagctgacgggtttc

SEQ ID NO: 950 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcagaccgattctgttaaatggtggggtacgaactactacatcaaggttcgtgcgggtgacaacaagtatatgcacctgaaagtgtttaagagcctgtggtacaacagatactacaacgatttcgaagatttggtgctgacgggctaccaggttgacaagaacaaagatgacgagctgacgggtttc

SEQ ID NO: 951 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccatcgtaaacatttccatcagttcaccggtacgaactactacatcaaggttcgtgcgggtgacaacaagtatatgcacctgaaagtgtttaagagcctgtctaacgattactacgaaacctacctggaagatttggtgctgacgggctaccaggttgacaagaacaaagatgacgagctgacgggtttc

SEQ ID NO: 952 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgttgttctgttcgcatctggttgggaaggtacgaactactacatcaaggttcgtgcgggtgacaacaagtatatgcacctgaaagtgtttaagagcctgatccagtggagagatcataaaccaagagaagatttggtgctgacgggctaccaggttgacaagaacaaagatgacgagctgacgggtttc

SEQ ID NO: 953 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctacgataacgttaaagaatacccatggggtacgaactactacatcaaggttcgtgcgggtgacaacaagtatatgcacctgaaagtgtttaagagcctgaaatggtacgatccaaccatcagaccagaagatttggtgctgacgggctaccaagttgacaagaacaaagatgacgagctgacgggtttc

SEQ ID NO: 954 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatccaggattggaccgttggttgggaaggtacgaactactacatcaaggttcgtgcgggtgacaacaagtatatgcacctgaaagtgtttaagagcctgagaatcggtagatctaccaacctgtgggaagatttggtgctgacgggctaccaggttgacaagaacaaagatgacgagctgacgggtttc

SEQ ID NO: 955 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcccacaggaaaacgcaccatgggttctgggtacgaactactacatcaaggttcgtgcgggtgacaacaagtatatgcacctgaaagtgtttaagagcctgaactgggatcagccagataccgttctggaagatttggtgctgacgggctaccaggttgacaagaacaaagatgacgagctgacgggtttc

SEQ ID NO: 956 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcttccagtctaccgattctttctggcatggtacgaactactacatcaaggttcgtgcgggtgacaacaagtatatgcacctgaaagtgtttaagagcctgttcgttgaaaactggcagaaaagaaccgaagatttggtgctgacgggctaccaggttgacaagaacaaagatgacgagctgacgggtttc

SEQ ID NO: 957 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcggtgttaaatctaccttccattggtctggtacgaactactacatcaaggttcgtgcgggtgacaacaagtatatgcacctgaaagtgtttaagagcctgttcaacaaccatccactgtctccagatgaagatttggtgctgacgggctaccaggttgacaagaacaaagatgacgagctgacgggtttc

SEQ ID NO: 958 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgcgttaccatttcccagaattccatggtacgaactactacatcaaggttcgtgcgggtgacaacaagtatatgcacctgaaagtgtttaagagcctgaaccatctggcaatccatgaactgccagaagatttggtgctgacgggctaccaggttgacaagaacaaagatgacgagctgacgggtttc

SEQ ID NO: 959 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaccgaaaacaactcttggtggccaatcggtacgaactactacatcaaggttcgtgcgggtgacaacaagtatatgcacctgaaagtgtttaagagcctgaactggttctctgatgaagcaccatacgaagatttggtgctgacgggctaccaggttgacaagaacaaagatgacgagctgacgggtttc

SEQ ID NO: 960 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgcgtcatccagttcaggatgatgatggtacgaactactacatcaaggttcgtgcgggtgacaacaagtatatgcacctgaaagtgtttaagagcctggcaggtgttaacagatggaaatggatcgaagatttggtgctgacgggctaccaggttgacaagaacaaagatgacgagctgacgggtttc

SEQ ID NO: 961 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgaagaacgtcgtatctggaactggaccggtacgaactactacatcaaggttcgtgcgggtgacaacaagtatatgcacctgaaagtgtttaagagcctgttcaccgcatctgatacccatccagcagaagatttggtgctgacgggctaccaggttgacaagaacaaagatgacgagctgacgggtttc

SEQ ID NO: 962 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgttggtgttggtcgtgatttcccaaaaggtacgaactactacatcaaggttcgtgcgggtgacaacaagtatatgcacctgaaagtgtttaagagcctgtacatccattacctgaaagttaacgaagaagatttggtgctgacgggctaccaggttgacaagaacaaagatgacgagctgacgggtttc

SEQ ID NO: 963 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgctgtggaccccaccatacgttgatggtacgaactactacatcaaggttcgtgcgggtgacaacaagtatatgcacctgaaagtgtttaagagcctgggttggaacgatgatcattctgttggtgaagatttggtgctgacgggctaccaggttgacaagaacaaagatgacgagctgacgggtttc

SEQ ID NO: 964 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctggaaaccacaggattctgttaactggggtacgaactactacatcaaggttcgtgcgggtgacaacaagtatatgcacctgaaagtgtttaagagcctggattgggatggtgcagcaggtgatgcagaagatttggtgctgacgggctaccaggttgacaagaacaaagatgacgagctgacgggtttc

SEQ ID NO: 965 - DNA, 342 bp, synthetic construct
  source 1..342: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctcttctccactgcattgggaactgtggcattggtctggtacgaactactacatcaaggttcgtgcgggtgacaacaagtatatgcacctgaaagtgtttaagagcctggaactgaacaacaaagatcagaacgatgaagatttggtgctgacgggctaccaggttgacaagaacaaagatgacgagctgacgggtttc

SEQ ID NO: 966 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtgatcatgaactggttaaccagtggggtacgaactactacatcaaggttcgtgcgggtgacaacaagtatatgcacctgaaagtgtttaagagcctgttcggtcagccagttcagggtaacaccgaagatttggtgctgacgggctaccaggttgacaagaacaaagatgacgagctgacgggtttc

SEQ ID NO: 967 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctacggtccacatcatcagacctacgcaggtacgaactactacatcaaggttcgtgcgggtgacaacaagtatatgcacctgaaagtgtttaagagcctgtacgcactgcatatcaacagatggccagaagatttggtgctgacgggctaccaggttgacaagaacaaagatgacgagctgacgggtttc

SEQ ID NO: 968 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccagcgtgcacacttcaaacagtggtacggtacgaactactacatcaaggttcgtgcgggtgacaacaagtatatgcacctgaaagtgtttaagagcctgccaggtgaaccacatcatatcctggatgaagatttggtgctgacgggctaccaggttgacaagaacaaagatgacgagctgacgggtttc

SEQ ID NO: 969 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatccgtcatgttatctggtcttactctggtacgaactactacatcaaggttcgtgcgggtgacaacaagtatatgcacctgaaagtgtttaagagcctgtgggaaagaaacgcatctctgtctgaagaagatttggtgctgacgggctaccaagttgacaagaacaaagatgacgagctgacgggtttc

SEQ ID NO: 970 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctaccatggtccaccaaaagaatggctgggtacgaactactacatcaaggttcgtgcgggtgacaacaagtatatgcacctgaaagtgtttaagagcctgttcttctggaaaggtaacggttacagagaagatttggtgctgacgggctaccaggttgacaagaacaaagatgacgagctgacgggtttc

SEQ ID NO: 971 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctctctgcgtccacatgaacagctgtacggtacgaactactacatcaaggttcgtgcgggtgacaacaagtatatgcacctgaaagtgtttaagagcctgcatctgaaactgtggaaaatcccaaacgaagatttggtgctgacgggctaccaggttgacaagaacaaagatgacgagctgacgggtttc

SEQ ID NO: 972 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgttttcctgtctgaaatccattggtacggtacgaactactacatcaaggttcgtgcgggtgacaacaagtatatgcacctgaaagtgtttaagagcctgagaatcaacaactctggtctgtctagagaagatttggtgctgacgggctaccaggttgacaagaacaaagatgacgagctgacgggtttc

SEQ ID NO: 973 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtgcagatgttaacgttatctggtggggtacgaactactacatcaaggttcgtgcgggtgacaacaagtatatgcacctgaaagtgtttaagagcctgccagttaaatacaaagaaatcctgctggaagatttggtgctgacgggctaccaggttgacaagaacaaagatgacgagctgacgggtttc

SEQ ID NO: 974 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgataccaaaaccccatggctgtggaccggtacgaactactacatcaaggttcgtgcgggtgacaacaagtatatgcacctgaaagtgtttaagagcctgttcctgaaaatcctggcagaagatgatgaagatttggtgctgacgggctaccaggttgacaagaacaaagatgacgagctgacgggtttc

SEQ ID NO: 975 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccagtggaaaaaaacccagtacttcttcggtacgaactactacatcaaggttcgtgcgggtgacaacaagtatatgcacctgaaagtgtttaagagcctgagaatcggttacctggaattcgaaatcgaagatttggtgctgacgggctaccaggttgacaagaacaaagatgacgagctgacgggtttc

SEQ ID NO: 976 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatcgcaaaagaagaaaaccattggcgtggtacgaactactacatcaaggttcgtgcgggtgacaacaagtatatgcacctgaaagtgtttaagagcctgcattactaccaggatgttctgtacagagaagatttggtgctgacgggctaccaggttgacaagaacaaagatgacgagctgacgggtttc

SEQ ID NO: 977 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctgggaatacgcaggtgattacctggaaggtacgaactactacatcaaggttcgtgcgggtgacaacaagtatatgcacctgaaagtgtttaagagcctgttccatggtatctggtgggcaagagttgaagatttggtgctgacgggctaccaggttgacaagaacaaagatgacgagctgacgggtttc

SEQ ID NO: 978 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcacccatccagaagatgcatactcttggggtacgaactactacatcaaggttcgtgcgggtgacaacaagtatatgcacctgaaagtgtttaagagcctgctgaaaccaggtggtctgctggatttcgaagatttggtgctgacgggctaccaggttgacaagaacaaagatgacgagctgacgggtttc

SEQ ID NO: 979 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctacgcattcatcaccccagttacccatggtacgaactactacatcaaggttcgtgcgggtgacaacaagtatatgcacctgaaagtgtttaagagcctgtggctgaacaacttcggtaaaaacttcgaagatttggtgctgacgggctaccaggttgacaagaacaaagatgacgagctgacgggtttc

SEQ ID NO: 980 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaacagcataccaaagaatgggcaccaggtacgaactactacatcaaggttcgtgcgggtgacaacaagtatatgcacctgaaagtgtttaagagcctgtggaaccatctgtaccattacccattcgaagatttggtgctgacgggctaccaggttgacaagaacaaagatgacgagctgacgggtttc

SEQ ID NO: 981 - DNA, 333 bp, synthetic construct
  source 1..333: mol_type = other DNA
atgatcccaggtggcctgagcgaagcagaaccagcaaccccagagattcaagaaattgttgacaaagtgaagccgcagctggaagagaaaaccggcgaaacctacggcaaactggaagccgtccagtataagactcaagtcgtcatcaaaatccatcatggttcttgggaaggtacgaactactacatcaaggttcgtgcgggtgacaacaagtatatgcacctgaaagtgtttaagagcctgctgttctggcagaaacagttctacagagaagatttggtgctgacgggctaccaggttgacaagaacaaagatgacgagctgacgggtttc

SEQ ID NO: 982 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtgttttcttcccagaattcccaggtacgaactactacatcaaggttcgtgcgggtgacaacaagtatatgcacctgaaagtgtttaagagcctggaacagccaatcgaaggtgatctgtgggaagatttggtgctgacgggctaccaggttgacaagaacaaagatgacgagctgacgggtttc

SEQ ID NO: 983 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcttcaccgaagttaaaccattcgttgaaggtacgaactactacatcaaggttcgtgcgggtgacaacaagtatatgcacctgaaagtgtttaagagcctgtcttggaacgcaccattcccagcaggtgaagatttggtgctgacgggctaccaggttgacaagaacaaagatgacgagctgacgggtttc

SEQ ID NO: 984 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaaaaatacgttatcttcttctacgaaggtacgaactactacatcaaggttcgtgcgggtgacaacaagtatatgcacctgaaagtgtttaagagcctgttcgttaacaaaggtggttctgatgatgaagatttggtgctgacgggctaccaggttgacaagaacaaagatgacgagctgacgggtttc

SEQ ID NO: 985 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctggcgtgtttacttcccagatttcaaaggtacgaactactacatcaaggttcgtgcgggtgacaacaagtatatgcacctgaaagtgtttaagagcctggatgaagttgatgaaatcccacatttcgaagatttggtgctgacgggctaccaggttgacaagaacaaagatgacgagctgacgggtttc

SEQ ID NO: 986 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcccactgaccatcccaccattcgttgaaggtacgaactactacatcaaggttcgtgcgggtgacaacaagtatatgcacctgaaagtgtttaagagcctgggttggcagcaggttaacgatgttaaagaagatttggtgctgacgggctaccaggttgacaagaacaaagatgacgagctgacgggtttc

SEQ ID NO: 987 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaaccgttggaacatccgtgaattcgaaggtacgaactactacatcaaggttcgtgcgggtgacaacaagtatatgcacctgaaagtgtttaagagcctgatcaacgttccagttaccgaactgctggaagatttggtgctgacgggctaccaggttgacaagaacaaagatgacgagctgacgggtttc

SEQ ID NO: 988 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatccaggatgttgattggcgtctgtggggtacgaactactacatcaaggttcgtgcgggtgacaacaagtatatgcacctgaaagtgtttaagagcctggttccatctgattggtctaaaaccatcgaagatttggtgctgacgggctaccaggttgacaagaacaaagatgacgagctgacgggtttc

SEQ ID NO: 989 - DNA, 333 bp, synthetic construct
  source 1..333: mol_type = other DNA
atgatcccaggtggcctgagcgaagcagaaccagcaaccccagagattcaagaaattgttgacaaagtgaagccgcagctggaagagaaaaccggcgaaacctacggcaaactggaagccgtccagtataagactcaagtcgtccagcgtgatcatttcccagaattcccaggtacgaactactacatcaaggttcgtgcgggtgacaacaagtatatgcacctgaaagtgtttaagagcctgggtcatgaactgtacgcactgtacgcagaagatttggtgctgacgggctaccaggttgacaagaacaaagatgacgagctgacgggtttc

SEQ ID NO: 990 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcttcccatgggatcaggaaaactactacggtacgaactactacatcaaggttcgtgcgggtgacaacaagtatatgcacctgaaagtgtttaagagcctgatcaaagaaccattcatcaaaaacgttgaagatttggtgctgacgggctaccaggttgacaagaacaaagatgacgagctgacgggtttc

SEQ ID NO: 991 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcacccgtcgtcatttcgttcagttcaccggtacgaactactacatcaaggttcgtgcgggtgacaacaagtatatgcacctgaaagtgtttaagagcctggatgaaccagcaaccccaacctacctggaagatttggtgctgacgggctaccaggttgacaagaacaaagatgacgagctgacgggtttc

SEQ ID NO: 992 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgatctgcataccccacattacgcaccaggtacgaactactacatcaaggttcgtgcgggtgacaacaagtatatgcacctgaaagtgtttaagagcctgtggaacaccctgctggaattcggtggtgaagatttggtgctgacgggctaccaggttgacaagaacaaagatgacgagctgacgggtttc

SEQ ID NO: 993 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctacaaacatgatgatgaatacggtttcggtacgaactactacatcaaggttcgtgcgggtgacaacaagtatatgcacctgaaagtgtttaagagcctgaaatggaccaacagagcaaacaaaccagaagatttggtgctgacgggctaccaggttgacaagaacaaagatgacgagctgacgggtttc

SEQ ID NO: 994 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctacgttcgttggcatgaatacggtttcggtacgaactactacatcaaggttcgtgcgggtgacaacaagtatatgcacctgaaagtgtttaagagcctgtctaaccagctgatcccacagccaatcgaagatttggtgctgacgggctaccaggttgacaagaacaaagatgacgagctgacgggtttc

SEQ ID NO: 995 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgtttggacctctgatgatgcatggcagggtacgaactactacatcaaggttcgtgcgggtgacaacaagtatatgcacctgaaagtgtttaagagcctgttcaaattcgaaatctacaaacataccgaagatttggtgctgacgggctaccaggttgacaagaacaaagatgacgagctgacgggtttc

SEQ ID NO: 996 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccattcttcttctaaaccatacatccatggtacgaactactacatcaaggttcgtgcgggtgacaacaagtatatgcacctgaaagtgtttaagagcctgaactggaacagaccatacaacgatctggaagatttggtgctgacgggctaccaagttgacaagaacaaagatgacgagctgacgggtttc

SEQ ID NO: 997 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccatgtttctaacttcatcatcccacatggtacgaactactacatcaaggttcgtgcgggtgacaacaagtatatgcacctgaaagtgtttaagagcctgaactgggaaggtaacgatctggcaaccgaagatttggtgctgacgggctaccaggttgacaagaacaaagatgacgagctgacgggtttc

SEQ ID NO: 998 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgcacgttggcattggttccatcatttcggtacgaactactacatcaaggttcgtgcgggtgacaacaagtatatgcacctgaaagtgtttaagagcctgaacccaatcaacggtcaggaaacctgggaagatttggtgctgacgggctaccaggttgacaagaacaaagatgacgagctgacgggtttc

SEQ ID NO: 999 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcaccaaacattctgcaaccttcttccatggtacgaactactacatcaaggttcgtgcgggtgacaacaagtatatgcacctgaaagtgtttaagagcctgatcggtaacggtcagtactggtacagagaagatttggtgctgacgggctaccaggttgacaagaacaaagatgacgagctgacgggtttc

SEQ ID NO: 1000 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgcgttaccatttccagcatttcccaggtacgaactactacatcaaggttcgtgcgggtgacaacaagtatatgcacctgaaagtgtttaagagcctggatggtcagggtgcactgtctctgccagaagatttggtgctgacgggctaccaggttgacaagaacaaagatgacgagctgacgggtttc

SEQ ID NO: 1001 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtaactctggtgtttgggaattcgaaggtacgaactactacatcaaggttcgtgcgggtgacaacaagtatatgcacctgaaagtgtttaagagcctgggtaaatacaaaaccaactggtacttcgaagatttggtgctgacgggctaccaggttgacaagaacaaagatgacgagctgacgggtttc

SEQ ID NO: 1002 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctggaacccaccatctctgccaatctctggtacgaactactacatcaaggttcgtgcgggtgacaacaagtatatgcacctgaaagtgtttaagagcctggcatgggataacaccgcaaaacatggtgaagatttggtgctgacgggctaccaggttgacaagaacaaagatgacgagctgacgggtttc

SEQ ID NO: 1003 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtgatggtaccttctgggatttctggggtacgaactactacatcaaggttcgtgcgggtgacaacaagtatatgcacctgaaagtgtttaagagcctgtctcaggattacaacggtggtcaggcagaagatttggtgctgacgggctaccaggttgacaagaacaaagatgacgagctgacgggtttc

SEQ ID NO: 1004 - DNA, 333 bp, synthetic construct; identical to SEQ ID NO: 933
  source 1..333: mol_type = other DNA

SEQ ID NO: 1005 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcataaaggtctgtggcagttcgaaggtacgaactactacatcaaggttcgtgcgggtgacaacaagtatatgcacctgaaagtgtttaagagcctgtctcatcagccatacagagcaaccatcgaagatttggtgctgacgggctaccaggttgacaagaacaaagatgacgagctgacgggtttc

SEQ ID NO: 1006 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgaccatcccaccaaccgcattctctggtacgaactactacatcaaggttcgtgcgggtgacaacaagtatatgcacctgaaagtgtttaagagcctgggttggaacctgcatgaagttgcactggaagatttggtgctgacgggctaccaggttgacaagaacaaagatgacgagctgacgggtttc

SEQ ID NO: 1007 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgctgtaccaggcaccattcgttcagggtacgaactactacatcaaggttcgtgcgggtgacaacaagtatatgcacctgaaagtgtttaagagcctgggttgggatctggaatactctcagaaagaagatttggtgctgacgggctaccaggttgacaagaacaaagatgacgagctgacgggtttc

SEQ ID NO: 1008 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctctcgtctgcatttcccaaacttccagggtacgaactactacatcaaggttcgtgcgggtgacaacaagtatatgcacctgaaagtgtttaagagcctgaacctggaacatccaaccgatgaaaccgaagatttggtgctgacgggctaccaggttgacaagaacaaagatgacgagctgacgggtttc

SEQ ID NO: 1009 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgttgaatggtctgaatctggttacgatggtacgaactactacatcaaggttcgtgcgggtgacaacaagtatatgcacctgaaagtgtttaagagcctgagactgaaatctaacgttaacatcgcagaagatttggtgctgacgggctaccaggttgacaagaacaaagatgacgagctgacgggtttc

SEQ ID NO: 1010 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctggaaacagatcaccctgccagttgatggtacgaactactacatcaaggttcgtgcgggtgacaacaagtatatgcacctgaaagtgtttaagagcctgcattgggatgaatacgaatacgatgaagaagatttggtgctgacgggctaccaggttgacaagaacaaagatgacgagctgacgggtttc

SEQ ID NO: 1011 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtctgggattctgaagcactgccaatcgatggtacgaactactacatcaaggttcgtgcgggtgacaacaagtatatgcacctgaaagtgtttaagagcctgagatggtggccatgggatgcagttggtgaagatttggtgctgacgggctaccaggttgacaagaacaaagatgacgagctgacgggtttc

SEQ ID NO: 1012 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcggttacctggaactgtggaaatggtctggtacgaactactacatcaaggttcgtgcgggtgacaacaagtatatgcacctgaaagtgtttaagagcctgtacaacctgcatcatccacataaagatgaagatttggtgctgacgggctaccaggttgacaagaacaaagatgacgagctgacgggtttc

SEQ ID NO: 1013 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgaacgtttccatttcccacagttcccaggtacgaactactacatcaaggttcgtgcgggtgacaacaagtatatgcacctgaaagtgtttaagagcctgccacatctggattacatctggaactgggaagatttggtgctgacgggctaccaggttgacaagaacaaagatgacgagctgacgggtttc

SEQ ID NO: 1014 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtgaacatttcccacgttggccaggtacgaactactacatcaaggttcgtgcgggtgacaacaagtatatgcacctgaaagtgtttaagagcctggatctgctgctgagaaaagaagaagttgaagatttggtgctgacgggctaccaggttgacaagaacaaagatgacgagctgacgggtttc

SEQ ID NO: 1015 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcctgcagctgccaccacgtccacatgatggtacgaactactacatcaaggttcgtgcgggtgacaacaagtatatgcacctgaaagtgtttaagagcctgaactgggaaccagcacagcatccagttgaagatttggtgctgacgggctaccaggttgacaagaacaaagatgacgagctgacgggtttc

SEQ ID NO: 1016 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatctactacccaatctggggtttccagggtacgaactactacatcaaggttcgtgcgggtgacaacaagtatatgcacctgaaagtgtttaagagcctgagatggtctaaagatggtgcaaccgcagaagatttggtgctgacgggctaccaggttgacaagaacaaagatgacgagctgacgggtttc

SEQ ID NO: 1017 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtgatccagatgttttcgttttctctggtacgaactactacatcaaggttcgtgcgggtgacaacaagtatatgcacctgaaagtgtttaagagcctgtggtactacaacgataaccagtctggtgaagatttggtgctgacgggctaccaggttgacaagaacaaagatgacgagctgacgggtttc

SEQ ID NO: 1018 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgcaaccgatcaggattggcgtaaatggggtacgaactactacatcaaggttcgtgcgggtgacaacaagtatatgcacctgaaagtgtttaagagcctgatcccactgaactggcagctgcaggaagaagatttggtgctgacgggctaccaggttgacaagaacaaagatgacgagctgacgggtttc

SEQ ID NO: 1019 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgcagttcgtccaatcttcaaatgggcaggtacgaactactacatcaaggttcgtgcgggtgacaacaagtatatgcacctgaaagtgtttaagagcctgttccatggtccactggataactacaccgaagatttggtgctgacgggctaccaggttgacaagaacaaagatgacgagctgacgggtttc

SEQ ID NO: 1020 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcacccgtccacatttcccaggttggaacggtacgaactactacatcaaggttcgtgcgggtgacaacaagtatatgcacctgaaagtgtttaagagcctggatttcgatcataacttcatcgcagatgaagatttggtgctgacgggctaccaggttgacaagaacaaagatgacgagctgacgggtttc

SEQ ID NO: 1021 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcatctctgatgtttctgttggttgggaaggtacgaactactacatcaaggttcgtgcgggtgacaacaagtatatgcacctgaaagtgtttaagagcctgagatctggtccagcacatgttagaagagaagatttggtgctgacgggctaccaggttgacaagaacaaagatgacgagctgacgggtttc

SEQ ID NO: 1022 - protein, 18 aa, Homo sapiens
MKWVTFISLLFLFSSAYS

SEQ ID NO: 1023 - protein, 22 aa, Homo sapiens
MDMRAPAGIFGFLLVLFPGYRS

SEQ ID NO: 1024 - protein, 19 aa, Homo sapiens
MTRLTVLALLAGLLASSRA

SEQ ID NO: 1025 - protein, 19 aa, Homo sapiens
MELGLSWIFLLAILKGVQC

SEQ ID NO: 1026 - protein, 19 aa, Homo sapiens
MELGLRWVFLVAILEGVQC

SEQ ID NO: 1027 - protein, 19 aa, Homo sapiens
MKHLWFFLLLVAAPRWVLS

SEQ ID NO: 1028 - protein, 19 aa, Homo sapiens
MDWTWRILFLVAAATGAHS

SEQ ID NO: 1029 - protein, 19 aa, Homo sapiens
MDWTWRFLFVVAAATGVQS

SEQ ID NO: 1030 - protein, 19 aa, Homo sapiens
MEFGLSWLFLVAILKGVQC

SEQ ID NO: 1031 - protein, 19 aa, Homo sapiens
MEFGLSWVFLVALFRGVQC

SEQ ID NO: 1032 - protein, 26 aa, Homo sapiens
MDLLHKNMKHLWFFLLLVAAPRWVLS

SEQ ID NO: 1033 - protein, 22 aa, Homo sapiens
MDMRVPAQLLGLLLLWLSGARC

SEQ ID NO: 1034 - protein, 22 aa, Homo sapiens
MKYLLPTAAAGLLLLAAQPAMA

SEQ ID NO: 1035 - protein, 17 aa, unidentified
  source 1..17: note = Gaussia luciferase
MGVKVLFALICIAVAEA

SEQ ID NO: 1036 - protein, 18 aa, Homo sapiens
MAFLWLLSCWALLGTTFG

SEQ ID NO: 1037 - protein, 14 aa, Homo sapiens
MQLLSCIALILALV

SEQ ID NO: 1038 - protein, 15 aa, Homo sapiens
MNLLLILTFVAAAVA

SEQ ID NO: 1039 - protein, 16 aa, Homo sapiens
MPLLLLLPLLWAGALA

SEQ ID NO: 1040 - protein, 30 aa, Homo sapiens
MDSKGSSQKGSRLLLLLVVSNLLLCQGVVS

SEQ ID NO: 1041 - protein, 23 aa, Homo sapiens
MDAMKRGLCCVLLLCGAVFVSPS

SEQ ID NO: 1042 - protein, 16 aa, synthetic construct
MLLLLLLLLLLALALA

SEQ ID NO: 1043 - protein, 21 aa, synthetic construct
MWWRLWWLLLLLLLLWPMVWA

SEQ ID NO: 1044 - protein, 5 aa, synthetic construct
  REPEAT 1..5
GGGGS

SEQ ID NO: 1045 - protein, 8 aa, synthetic construct
GGGGGGGG

SEQ ID NO: 1046 - protein, 6 aa, synthetic construct
GGGGGG

SEQ ID NO: 1047 - protein, 18 aa, synthetic construct
KESGSVSSEQLAQFRSLD

SEQ ID NO: 1048 - protein, 14 aa, synthetic construct
EGKSSGSGSESKST

SEQ ID NO: 1049 - protein, 12 aa, synthetic construct
GSAGSAAGSGEF

SEQ ID NO: 1050 - protein, 5 aa, synthetic construct
  REPEAT 1..5
EAAAK

SEQ ID NO: 1051 - protein, 46 aa, synthetic construct
AEAAAKEAAAKEAAAKEAAAKALEAEAAAKEAAAKEAAAKEAAAKA

SEQ ID NO: 1052 - protein, 5 aa, synthetic construct
PAPAP

SEQ ID NO: 1053 - protein, 12 aa, synthetic construct
AEAAAKEAAAKA

SEQ ID NO: 1054 - protein, 10 aa, synthetic construct
  REPEAT 9..10: note = May repeat up to 12 times
APAPAPAPAP

SEQ ID NO: 1055 - protein, 6 aa, synthetic construct
  REPEAT 2..6
AEAAAK

SEQ ID NO: 1056 - protein, 4 aa, synthetic construct
GGSG

SEQ ID NO: 1057 - protein, 4 aa, synthetic construct
GSGS

SEQ ID NO: 1058 - protein, 4 aa, synthetic construct
GGGS

SEQ ID NO: 1059 - protein, 4 aa, synthetic construct
  REPEAT 2..4
SGGS

SEQ ID NO: 1060 - protein, 7 aa, synthetic construct
GGGSGGG

SEQ ID NO: 1061 - protein, 8 aa, synthetic construct
ESGGGGVT

SEQ ID NO: 1062 - protein, 9 aa, synthetic construct
LESGGGGVT

SEQ ID NO: 1063 - protein, 6 aa, synthetic construct
GRAQVT

SEQ ID NO: 1064 - protein, 6 aa, synthetic construct
WRAQVT

SEQ ID NO: 1065 - protein, 8 aa, synthetic construct
ARGRAQVT

SEQ ID NO: 1066 - protein, 33 aa, synthetic construct
SGTTSGTTRLLSGHTCFTLTGLLGTLVTMGLLT

SEQ ID NO: 1067 - protein, 26 aa, synthetic construct
SGTSPGLSAGATVGIMIGVLVGVALI

SEQ ID NO: 1068 - protein, 24 aa, synthetic construct
SAPVLSAVATVGITIGVLARVALI

SEQ ID NO: 1069 - protein, 24 aa, synthetic construct
SSPDLSAGTAVSIMIGVLAGMALI

SEQ ID NO: 1070 - protein, 25 aa, synthetic construct
TLGGNSASYTFVSLLFSAVTLLLLC

SEQ ID NO: 1071 - protein, 26 aa, synthetic construct
SGTSPGLSAGATVGIMIGVLVGVALI

SEQ ID NO: 1072 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1073 - protein, 16 aa, Homo sapiens
DKTHTCPPCPAPELLG

SEQ ID NO: 1074 - protein, 16 aa, synthetic construct
GSTHTCPPCPAPELLG

SEQ ID NO: 1075 - protein, 21 aa, synthetic construct
EPKSCDKTHTCPPCPAPELLG

SEQ ID NO: 1076 - protein, 24 aa, synthetic construct
EPKSCDKTHTCPPCPAPELLGGPS

SEQ ID NO: 1077 - protein, 24 aa, synthetic construct
EPKSSDKTHTCPPCPAPELLGGPS

SEQ ID NO: 1078 - protein, 24 aa, synthetic construct
EPKSSDKTHTCPPCPAPELLGGSS

SEQ ID NO: 1079 - protein, 24 aa, synthetic construct
EPKSSGSTHTCPPCPAPELLGGSS

SEQ ID NO: 1080 - protein, 19 aa, synthetic construct
DKTHTCPPCPAPELLGGPS

SEQ ID NO: 1081 - protein, 19 aa, synthetic construct
DKTHTCPPCPAPELLGGSS

SEQ ID NO: 1082 - protein, 227 aa, Homo sapiens; identical to SEQ ID NO: 1072

SEQ ID NO: 1083 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 1084 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1085 - protein, 227 aa, synthetic construct
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1086 - protein, 232 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1087 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1088 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1089 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1090 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1091 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1092 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYHSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1093 - protein, 233 aa, Homo sapiens
DKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1094 - protein, 233 aa, synthetic construct
DKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1095 - protein, 233 aa, synthetic construct
SEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1096 - protein, 585 aa, Homo sapiens [sequence omitted]

SEQ ID NO: 1097 - protein, 6 aa, synthetic construct
RGVFRR

SEQ ID NO: 1098 - DNA, 12 bp, synthetic construct
  source 1..12: mol_type = other DNA
accgttgccggt

SEQ ID NO: 1099 - skipped

SEQ ID NO: 1100 - protein, 9 aa, synthetic construct
ARKHFPQWP

SEQ ID NO: 1101 - protein, 9 aa, synthetic construct
RAKHFPQWP

SEQ ID NO: 1102 - protein, 9 aa, synthetic construct
RRAHFPQWP

SEQ ID NO: 1103 - protein, 9 aa, synthetic construct
RRKAFPQWP

SEQ ID NO: 1104 - protein, 9 aa, synthetic construct
RRKHAPQWP

SEQ ID NO: 1105 - protein, 9 aa, synthetic construct
RRKHFAQWP

SEQ ID NO: 1106 - protein, 9 aa, synthetic construct
RRKHFPAWP

SEQ ID NO: 1107 - protein, 9 aa, synthetic construct
RRKHFPQAP

SEQ ID NO: 1108 - protein, 9 aa, synthetic construct
RRKHFPQWA

SEQ ID NO: 1109 - protein, 9 aa, synthetic construct
ALQPREVFQ

SEQ ID NO: 1110 - protein, 9 aa, synthetic construct
DAQPREVFQ

SEQ ID NO: 1111 - protein, 9 aa, synthetic construct
DLAPREVFQ

SEQ ID NO: 1112 - protein, 9 aa, synthetic construct
DLQAREVFQ

SEQ ID NO: 1113 - protein, 9 aa, synthetic construct
DLQPAEVFQ

SEQ ID NO: 1114 - protein, 9 aa, synthetic construct
DLQPRAVFQ

SEQ ID NO: 1115 - protein, 9 aa, synthetic construct
DLQPREAFQ

SEQ ID NO: 1116 - protein, 9 aa, synthetic construct
DLQPREVAQ

SEQ ID NO: 1117 - protein, 9 aa, synthetic construct
DLQPREVFA

SEQ ID NO: 1118 - protein, 112 aa, synthetic construct
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 1119 - protein, 112 aa, synthetic construct
MIPRGLSEAKPATPEIQEIVDKVKPQLEEKTNETYGKLEAVQYKTQVLAKEHGPDSWWSTNYYIKVRAGDNKYMHLKVFNGPQEKNQWVEEADRVLTGYQVDKNKDDELTGF

SEQ ID NO: 1120 - protein, 25 aa, synthetic construct
GGGGSGGGGSGGGGSGGGGSGGGGS

SEQ ID NO: 1121 - protein, 366 aa, synthetic construct
MIPGGLSEAKPATPEIQEIVDKVKPQLEEKTGETYGKLEAVQYKTQVVTQAIPWREWGTNYYIKVRAGDNKYMHLKVFKSLIPEWPKDASEDLVLTGYQVDKNKDDELTGFAAAGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1122 - protein, 253 aa, synthetic construct
MIPGGLSEAKPATPEIQEIVDKVKPQLEEKTGETYGKLEAVQYKTQVVRRKHFPQWPGTNYYIKVRAGDNKYMHLKVFKSLDLQPREVFQEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVRRKHFPQWPGTNYYIKVRAGDNKYMHLKVFKSLDLQPREVFQEDLVLTGYQVDKNKDDELTGF

SEQ ID NO: 1123 - DNA, 336 bp, synthetic construct
  source 1..336: mol_type = other DNA
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcacgtgaaggtcgtcaggattgggttctgtccaccaactattacattaaggttcgtgccggtgacaataagtatatgcacctgaaagtgttcaacggcccgtgggttccatttccacatcagcagctggcggaccgtgttctgaccggttaccaggttgacaagaacaaagatgacgagctgacgggtttc

SEQ ID NO: 1124 - DNA, 336 bp, synthetic construct
  source 1..336: mol_type = other DNA
atgatcccgcgtggcctgtctgaagctaaaccagcaactccggaaattcaagagatcgtcgataaggtgaaaccgcagctggaagagaaaacgaacgaaacctacggtaagctggaagcggtccagtacaaaacccaagtgctagcaaaagaacatggtccagattcttggtggtccaccaactattacattaaggttcgtgccggtgacaataagtatatgcacctgaaagtgttcaacggcccgcaggaaaaaaaccagtgggttgaagaagcggaccgtgttctgaccggttaccaggttgacaagaacaaagatgacgagctgacgggtttc

SEQ ID NO: 1125 - protein, 111 aa, synthetic construct
MIPGGLSEAKPATPEIQEIVDKVKPQLEEKTGETYGKLEAVQYKTQVVARKHFPQWPGTNYYIKVRAGDNKYMHLKVFKSLDLQPREVFQEDLVLTGYQVDKNKDDELTGF

SEQ ID NO: 1126 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtcgctcgtaaacatttcccacagtggccaggtacgaactactacatcaaggttcgtgcgggtgacaacaagtatatgcacctgaaagtgtttaagagcctggatctgcagccaagagaagttttccaggaagatttggtgctgacgggctaccaggttgacaagaacaaagatgacgagctgacgggtttc

SEQ ID NO: 1127 - protein, 111 aa, synthetic construct
MIPGGLSEAKPATPEIQEIVDKVKPQLEEKTGETYGKLEAVQYKTQVVRAKHFPQWPGTNYYIKVRAGDNKYMHLKVFKSLDLQPREVFQEDLVLTGYQVDKNKDDELTGF

SEQ ID NO: 1128 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtgctaaacatttcccacagtggccaggtacgaactactacatcaaggttcgtgcgggtgacaacaagtatatgcacctgaaagtgtttaagagcctggatctgcagccaagagaagttttccaggaagatttggtgctgacgggctaccaggttgacaagaacaaagatgacgagctgacgggtttc

SEQ ID NO: 1129 - protein, 111 aa, synthetic construct
MIPGGLSEAKPATPEIQEIVDKVKPQLEEKTGETYGKLEAVQYKTQVVRRAHFPQWPGTNYYIKVRAGDNKYMHLKVFKSLDLQPREVFQEDLVLTGYQVDKNKDDELTGF

SEQ ID NO: 1130 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtgcacatttcccacagtggccaggtacgaactactacatcaaggttcgtgcgggtgacaacaagtatatgcacctgaaagtgtttaagagcctggatctgcagccaagagaagttttccaggaagatttggtgctgacgggctaccaggttgacaagaacaaagatgacgagctgacgggtttc

SEQ ID NO: 1131 - protein, 111 aa, synthetic construct
MIPGGLSEAKPATPEIQEIVDKVKPQLEEKTGETYGKLEAVQYKTQVVRRKAFPQWPGTNYYIKVRAGDNKYMHLKVFKSLDLQPREVFQEDLVLTGYQVDKNKDDELTGF

SEQ ID NO: 1132 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaagctttcccacagtggccaggtacgaactactacatcaaggttcgtgcgggtgacaacaagtatatgcacctgaaagtgtttaagagcctggatctgcagccaagagaagttttccaggaagatttggtgctgacgggctaccaggttgacaagaacaaagatgacgagctgacgggtttc

SEQ ID NO: 1133 - protein, 111 aa, synthetic construct
MIPGGLSEAKPATPEIQEIVDKVKPQLEEKTGETYGKLEAVQYKTQVVRRKHAPQWPGTNYYIKVRAGDNKYMHLKVFKSLDLQPREVFQEDLVLTGYQVDKNKDDELTGF

SEQ ID NO: 1134 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatgccccacagtggccaggtacgaactactacatcaaggttcgtgcgggtgacaacaagtatatgcacctgaaagtgtttaagagcctggatctgcagccaagagaagttttccaggaagatttggtgctgacgggctaccaggttgacaagaacaaagatgacgagctgacgggtttc

SEQ ID NO: 1135 - protein, 111 aa, synthetic construct
MIPGGLSEAKPATPEIQEIVDKVKPQLEEKTGETYGKLEAVQYKTQVVRRKHFAQWPGTNYYIKVRAGDNKYMHLKVFKSLDLQPREVFQEDLVLTGYQVDKNKDDELTGF

SEQ ID NO: 1136 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcgcacagtggccaggtacgaactactacatcaaggttcgtgcgggtgacaacaagtatatgcacctgaaagtgtttaagagcctggatctgcagccaagagaagttttccaggaagatttggtgctgacgggctaccaggttgacaagaacaaagatgacgagctgacgggtttc

SEQ ID NO: 1137 - protein, 111 aa, synthetic construct
MIPGGLSEAKPATPEIQEIVDKVKPQLEEKTGETYGKLEAVQYKTQVVRRKHFPAWPGTNYYIKVRAGDNKYMHLKVFKSLDLQPREVFQEDLVLTGYQVDKNKDDELTGF

SEQ ID NO: 1138 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcccagcgtggccaggtacgaactactacatcaaggttcgtgcgggtgacaacaagtatatgcacctgaaagtgtttaagagcctggatctgcagccaagagaagttttccaggaagatttggtgctgacgggctaccaggttgacaagaacaaagatgacgagctgacgggtttc

SEQ ID NO: 1139 - protein, 111 aa, synthetic construct
MIPGGLSEAKPATPEIQEIVDKVKPQLEEKTGETYGKLEAVQYKTQVVRRKHFPQAPGTNYYIKVRAGDNKYMHLKVFKSLDLQPREVFQEDLVLTGYQVDKNKDDELTGF

SEQ ID NO: 1140 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcccacaggcgccaggtacgaactactacatcaaggttcgtgcgggtgacaacaagtatatgcacctgaaagtgtttaagagcctggatctgcagccaagagaagttttccaggaagatttggtgctgacgggctaccaggttgacaagaacaaagatgacgagctgacgggtttc

SEQ ID NO: 1141 - protein, 111 aa, synthetic construct
MIPGGLSEAKPATPEIQEIVDKVKPQLEEKTGETYGKLEAVQYKTQVVRRKHFPQWAGTNYYIKVRAGDNKYMHLKVFKSLDLQPREVFQEDLVLTGYQVDKNKDDELTGF

SEQ ID NO: 1142 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcccacagtgggcaggtacgaactactacatcaaggttcgtgcgggtgacaacaagtatatgcacctgaaagtgtttaagagcctggatctgcagccaagagaagttttccaggaagatttggtgctgacgggctaccaggttgacaagaacaaagatgacgagctgacgggtttc

SEQ ID NO: 1143 - protein, 111 aa, synthetic construct
MIPGGLSEAKPATPEIQEIVDKVKPQLEEKTGETYGKLEAVQYKTQVVRRKHFPQWPGTNYYIKVRAGDNKYMHLKVFKSLALQPREVFQEDLVLTGYQVDKNKDDELTGF

SEQ ID NO: 1144 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcccacagtggccaggtacgaactactacatcaaggttcgtgcgggtgacaacaagtatatgcacctgaaagtgtttaagagcctggctctgcagccaagagaagttttccaggaagatttggtgctgacgggctaccaggttgacaagaacaaagatgacgagctgacgggtttc

SEQ ID NO: 1145 - protein, 111 aa, synthetic construct
MIPGGLSEAKPATPEIQEIVDKVKPQLEEKTGETYGKLEAVQYKTQVVRRKHFPQWPGTNYYIKVRAGDNKYMHLKVFKSLDLAPREVFQEDLVLTGYQVDKNKDDELTGF

SEQ ID NO: 1146 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcccacagtggccaggtacgaactactacatcaaggttcgtgcgggtgacaacaagtatatgcacctgaaagtgtttaagagcctggatctggcgccaagagaagttttccaggaagatttggtgctgacgggctaccaggttgacaagaacaaagatgacgagctgacgggtttc

SEQ ID NO: 1147 - protein, 111 aa, synthetic construct
MIPGGLSEAKPATPEIQEIVDKVKPQLEEKTGETYGKLEAVQYKTQVVRRKHFPQWPGTNYYIKVRAGDNKYMHLKVFKSLDLQAREVFQEDLVLTGYQVDKNKDDELTGF

SEQ ID NO: 1148 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcccacagtggccaggtacgaactactacatcaaggttcgtgcgggtgacaacaagtatatgcacctgaaagtgtttaagagcctggatctgcaggcaagagaagttttccaggaagatttggtgctgacgggctaccaggttgacaagaacaaagatgacgagctgacgggtttc

SEQ ID NO: 1149 - protein, 111 aa, synthetic construct
MIPGGLSEAKPATPEIQEIVDKVKPQLEEKTGETYGKLEAVQYKTQVVRRKHFPQWPGTNYYIKVRAGDNKYMHLKVFKSLDLQPAEVFQEDLVLTGYQVDKNKDDELTGF

SEQ ID NO: 1150 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcccacagtggccaggtacgaactactacatcaaggttcgtgcgggtgacaacaagtatatgcacctgaaagtgtttaagagcctggatctgcagccagcagaagttttccaggaagatttggtgctgacgggctaccaggttgacaagaacaaagatgacgagctgacgggtttc

SEQ ID NO: 1151 - protein, 111 aa, synthetic construct
MIPGGLSEAKPATPEIQEIVDKVKPQLEEKTGETYGKLEAVQYKTQVVRRKHFPQWPGTNYYIKVRAGDNKYMHLKVFKSLDLQPRAVFQEDLVLTGYQVDKNKDDELTGF

SEQ ID NO: 1152 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcccacagtggccaggtacgaactactacatcaaggttcgtgcgggtgacaacaagtatatgcacctgaaagtgtttaagagcctggatctgcagccaagagcagttttccaggaagatttggtgctgacgggctaccaggttgacaagaacaaagatgacgagctgacgggtttc

SEQ ID NO: 1153 - protein, 111 aa, synthetic construct
MIPGGLSEAKPATPEIQEIVDKVKPQLEEKTGETYGKLEAVQYKTQVVRRKHFPQWPGTNYYIKVRAGDNKYMHLKVFKSLDLQPREAFQEDLVLTGYQVDKNKDDELTGF

SEQ ID NO: 1154 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcccacagtggccaggtacgaactactacatcaaggttcgtgcgggtgacaacaagtatatgcacctgaaagtgtttaagagcctggatctgcagccaagagaagctttccaggaagatttggtgctgacgggctaccaggttgacaagaacaaagatgacgagctgacgggtttc

SEQ ID NO: 1155 - protein, 111 aa, synthetic construct
MIPGGLSEAKPATPEIQEIVDKVKPQLEEKTGETYGKLEAVQYKTQVVRRKHFPQWPGTNYYIKVRAGDNKYMHLKVFKSLDLQPREVAQEDLVLTGYQVDKNKDDELTGF

SEQ ID NO: 1156 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcccacagtggccaggtacgaactactacatcaaggttcgtgcgggtgacaacaagtatatgcacctgaaagtgtttaagagcctggatctgcagccaagagaagttgcccaggaagatttggtgctgacgggctaccaggttgacaagaacaaagatgacgagctgacgggtttc

SEQ ID NO: 1157 - protein, 111 aa, synthetic construct
MIPGGLSEAKPATPEIQEIVDKVKPQLEEKTGETYGKLEAVQYKTQVVRRKHFPQWPGTNYYIKVRAGDNKYMHLKVFKSLDLQPREVFAEDLVLTGYQVDKNKDDELTGF

SEQ ID NO: 1158 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcccacagtggccaggtacgaactactacatcaaggttcgtgcgggtgacaacaagtatatgcacctgaaagtgtttaagagcctggatctgcagccaagagaagttttcgcggaagatttggtgctgacgggctaccaggttgacaagaacaaagatgacgagctgacgggtttc

SEQ ID NO: 1159 - protein, 111 aa, synthetic construct
MIPGGLSEAKPATPEIQEIVDKVKPQLEEKTGETYGKLEAVQYKTQVDRRKHFPQWPGTNYYIKVRAGDNKYMHLKVFKSLDLQPREVFQEDLVLTGYQVDKNKDDELTGF

SEQ ID NO: 1160 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgatcgtcgtaaacatttcccacagtggccaggtacgaactactacatcaaggttcgtgcgggtgacaacaagtatatgcacctgaaagtgtttaagagcctggatctgcagccaagagaagttttccaggaagatttggtgctgacgggctaccaggttgacaagaacaaagatgacgagctgacgggtttc

SEQ ID NO: 1161 - protein, 111 aa, synthetic construct
MIPGGLSEAKPATPEIQEIVDKVKPQLEEKTGETYGKLEAVQYKTQVVRRKHFPQWPGTNYYIKVRAGDNKYMHLKVFKSLDAQPREVFQEDLVLTGYQVDKNKDDELTGF

SEQ ID NO: 1162 - DNA, 333 bp, synthetic construct
  source 1..333: mol_type = other DNA
atgatcccaggtggcctgagcgaagcaaaaccagcaaccccagagattcaagaaattgttgacaaagtgaagccgcagctggaagagaaaaccggcgaaacctacggcaaactggaagccgtccagtataagactcaagtcgtccgtcgtaaacatttcccacagtggccaggtacgaactactacatcaaggttcgtgcgggtgacaacaagtatatgcacctgaaagtgtttaagagcctggatgcgcagccaagagaagttttccaggaagatttggtgctgacgggctaccaggttgacaagaacaaagatgacgagctgacgggtttc

SEQ ID NO: 1163 - DNA, 1098 bp, synthetic construct
  source 1..1098: mol_type = other DNA
atgatccctggcggcctgtcggaagccaagccagccacaccggagattcaggaaatcgtggataaggtcaagccccagctggaggaaaagaccggcgaaacctacggcaaactggaagccgtccagtataagactcaagtcgtcacccaggcaatcccatggcgtgaatggggtacgaactactacatcaaagttcgtgcgggtgacaacaagtatatgcacctgaaagtgtttaagagcctgatcccagaatggccaaaagatgcatctgaagatttggtgctgacgggctaccaggttgacaagaacaaagatgacgagctgacgggtttcgcggccgccggtggtggagggtccggtggcgggggctcagggggcggtggcagcgggggtggcggatcggaacctaagtcctgcgacaagacccatacttgccctccgtgccccgccccggaactcctgggaggcccgtctgtgtttctgttcccgccaaaaccgaaggacaccctgatgatttcccggacgcctgaagtgacctgtgtggtggtcgatgtgtcccacgaggaccccgaagtcaagttcaattggtacgtggatggagtggaggtccacaacgccaagacaaagcccagggaggaacagtacaacagcacctaccgggtggtgtcagtgctgactgtgctgcaccaagactggctgaacggaaaggagtacaagtgcaaagtgtcgaacaaggccctgcctgcacccatcgaaaagactatcagcaaagctaagggacagccccgcgagccgcaagtctatacccttccaccttcgcgcgaggagatgaccaagaaccaagtgtccctgacttgcctcgtcaagggattctacccctccgacatcgcggtggaatgggagtccaatggccagccagaaaacaactacaagaccactcctccggtgctcgattcggatggaagcttcttcctgtactccaagttgacggtcgacaagtcaagatggcagcagggcaacgtgttcagctgttccgtgatgcacgaagccctgcacaaccattacacccagaagtccctctccctgtcaccggggaag

SEQ ID NO: 1164 - protein, 111 aa, synthetic construct; identical to SEQ ID NO: 593

SEQ ID NO: 1165 - DNA, 333 bp, synthetic construct
  source 1..333: mol_type = other DNA
atgattcccggcggactttcggaagccaagccagccactccggagattcaggaaatcgtggacaaggtcaaaccgcagctggaagaaaagaccggggaaacctacgggaagttggaggcggtccagtacaaaacccaggtcgtgcgcagaaaacacttcccccaatggcccggtaccaattactatatcaaggttcgcgccggagacaacaagtacatgcacctgaaagtgttcaagagcctggatctgcagcctcgcgaagtgttccaggaggacctggtgctcaccggataccaggtcgacaagaacaaggacgacgaactgacaggcttc

SEQ ID NO: 1166 - DNA, 759 bp, synthetic construct
  source 1..759: mol_type = other DNA
atgattccgggtggcctttccgaggccaagcctgccactcccgagatccaggagatcgtggataaggtcaagccacagctggaggaaaagactggagagacttacggaaagctcgaagccgtgcagtacaagacccaagtcgtgcggcggaagcacttcccgcaatggcctggaaccaactactacatcaaagtccgggccggggataacaagtatatgcatctcaaggtgttcaagtccttggacctccaaccgagggaggtctttcaggaggatctggtgctgacgggctaccaagtggacaagaataaggacgatgagctgaccggcttcgctgaagcggcagcgaaggaagccgcggcgaaagaagccgcagccaaggaggcagccgccaaagaggccgccgctaaggaagctgccgccaagatgattcccggcggactttcggaagccaagccagccactccggagattcaggaaatcgtggacaaggtcaaaccgcagctggaagaaaagaccggggaaacctacgggaagttggaggcggtccagtacaaaacccaggtcgtgcgcagaaaacacttcccccaatggcccggtaccaattactatatcaaggttcgcgccggagacaacaagtacatgcacctgaaagtgttcaagagcctggatctgcagcctcgcgaagtgttccaggaggacctggtgctcaccggataccaggtcgacaagaacaaggacgacgaactgacaggcttc

SEQ ID NO: 1167 - protein, 116 aa, synthetic construct
MAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA

SEQ ID NO: 1168 - DNA, 348 bp, synthetic construct
  source 1..348: mol_type = other DNA
atggcgttcaccgtcactgtcccgaaagacctgtatgtagtggagtatggtagcaatatgaccattgagtgcaagtttcctgtggagaaacagctggacctggcggcgttgattgtttactgggaaatggaagataagaatatcattcagtttgtgcatggtgaggaagatttgaaagttcaacacagctcctaccgccagcgtgcacgcctgctgaaagatcagctgagcctgggcaatgctgccctgcaaatcactgatgtgaagctgcaagatgcgggtgtttatcgttgcatgatcagctacggtggcgcagactacaagcgtatcaccgttaaagtcaatgcg

SEQ ID NO: 1169 - protein, 31 aa, synthetic construct
AEAAAKEAAAKEAAAKEAAAKEAAAKEAAAK

SEQ ID NO: 1170 - protein, 8 aa, synthetic construct
TGRGPSWV

SEQ ID NO: 1171 - protein, 8 aa, synthetic construct
SARGPSRW

SEQ ID NO: 1172 - protein, 8 aa, synthetic construct
TARGPSFK

SEQ ID NO: 1173 - protein, 8 aa, synthetic construct
LSGRSDNH

SEQ ID NO: 1174 - protein, 8 aa, synthetic construct
GGWHTGRN

SEQ ID NO: 1175 - protein, 8 aa, synthetic construct
HTGRSGAL

SEQ ID NO: 1176 - protein, 8 aa, synthetic construct
PLTGRSGG

SEQ ID NO: 1177 - protein, 8 aa, synthetic construct
AARGPAIH

SEQ ID NO: 1178 - protein, 8 aa, synthetic construct
RGPAFNPM

SEQ ID NO: 1179 - protein, 8 aa, synthetic construct
SSRGPAYL

SEQ ID NO: 1180 - protein, 8 aa, synthetic construct
RGPATPIM

SEQ ID NO: 1181 - protein, 4 aa, synthetic construct
RGPA

SEQ ID NO: 1182 - protein, 10 aa, synthetic construct
GGQPSGMWGW

SEQ ID NO: 1183 - protein, 10 aa, synthetic construct
FPRPLGITGL

SEQ ID NO: 1184 - protein, 10 aa, synthetic construct
VHMPLGFLGP

SEQ ID NO: 1185 - protein, 8 aa, synthetic construct
SPLTGRSG

SEQ ID NO: 1186 - protein, 8 aa, synthetic construct
SAGFSLPA

SEQ ID NO: 1187 - protein, 9 aa, synthetic construct
LAPLGLQRR

SEQ ID NO: 1188 - protein, 8 aa, synthetic construct
SGGPLGVR

SEQ ID NO: 1189 - protein, 4 aa, synthetic construct
PLGL

SEQ ID NO: 1190 - protein, 7 aa, synthetic construct
GPRSFGL

SEQ ID NO: 1191 - protein, 6 aa, synthetic construct
GPRSFG

SEQ ID NO: 1192 - protein, 8 aa, synthetic construct
ISSGLLSS

SEQ ID NO: 1193 - protein, 8 aa, synthetic construct
QNQALRMA

SEQ ID NO: 1194 - protein, 8 aa, synthetic construct
AQNLLGMV

SEQ ID NO: 1195 - protein, 8 aa, synthetic construct
STFPFGMF

SEQ ID NO: 1196 - protein, 8 aa, synthetic construct
PVGYTSSL

SEQ ID NO: 1197 - protein, 8 aa, synthetic construct
DWLYWPGI

SEQ ID NO: 1198 - protein, 8 aa, synthetic construct
MIAPVAYR

SEQ ID NO: 1199 - protein, 8 aa, synthetic construct
RPSPMWAY

SEQ ID NO: 1200 - protein, 8 aa, synthetic construct
WATPRPMR

SEQ ID NO: 1201 - protein, 8 aa, synthetic construct
FRLLDWQW

SEQ ID NO: 1202 - protein, 8 aa, synthetic construct
LKAAPRWA

SEQ ID NO: 1203 - protein, 8 aa, synthetic construct
GPSHLVLT

SEQ ID NO: 1204 - protein, 8 aa, synthetic construct
LPGGLSPW

SEQ ID NO: 1205 - protein, 8 aa, synthetic construct
MGLFSEAG

SEQ ID NO: 1206 - protein, 8 aa, synthetic construct
SPLPLRVP

SEQ ID NO: 1207 - protein, 8 aa, synthetic construct
RMHLRSLG

SEQ ID NO: 1208 - protein, 8 aa, synthetic construct
LAAPLGLL

SEQ ID NO: 1209 - protein, 8 aa, synthetic construct
AVGLLAPP

SEQ ID NO: 1210 - protein, 8 aa, synthetic construct
LLAPSHRA

SEQ ID NO: 1211 - protein, 8 aa, synthetic construct
PAGLWLDP

SEQ ID NO: 1212 - protein, 7 aa, synthetic construct
ISSGLSS